SEQUENCE LISTING (9 sequences)

SEQ ID NO: 1 - protein, 13 aa, Artificial sequence
  synthetic peptide
SEVNLDAEFRCKK

SEQ ID NO: 2 - protein, 13 aa, Artificial sequence
  synthetic peptide
SEVKMDAEFRCKK

SEQ ID NO: 3 - protein, 22 aa, Artificial sequence
  synthetic peptide
GLNIKTEEISEISYEVEFRCKK

SEQ ID NO: 4 - protein, 34 aa, Artificial sequence
  synthetic peptide
ADRGLTTRPGSGLTNIKTEEISEVNLDAEFRCKK

SEQ ID NO: 5 - protein, 33 aa, Artificial sequence
  synthetic peptide
FVNQHLCGSHLVEALYLVCGERGFFYTPKACKK

SEQ ID NO: 6 - protein, 33 aa, Artificial sequence
  synthetic peptide
CGGADRGLTTRPGSGLTNIKTEEISEVNLDAEF

SEQ ID NO: 7 - protein, 29 aa, Artificial sequence
  synthetic peptide
CGGADRGLTTRPGSGLTNIKTEEISEVNL

SEQ ID NO: 8 - protein, 9 aa, Artificial sequence
  synthetic peptide
SEVNLDAEF

SEQ ID NO: 9 - protein, 30 aa, Artificial sequence
  synthetic peptide
ADRGLTTRPGSGLTNIKTEEISEVNLDAEF

(19) United States
(12) Patent Application Publication
(10) Pub. No.: US 2006/0079533 A1
(43) Pub. Date: Apr. 13, 2006

(54) Methods of treating alzheimer's disease

(76) Inventors: James A. Nieman, Galesburg, MI (US); Lawrence Fang, Foster City, CA (US); barbara Jagodzinska, Redwood City, CA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/472,868

(22) PCT Filed: Mar. 21, 2002
(86) PCT No.: PCT/US02/09100

§ 371 Date: Feb. 2, 2004

Related U.S. Application Data
(60) Provisional application No. 60/278,371, filed on Mar. 23, 2001.
(60) Provisional application No. 60/308,729, filed on Jul. 30, 2001.

(51) Int. Cl. A61K 31/501; A61K 31/497; A61K 31/496; A61K 31/445; A61K 31/46

(52) U.S. Cl. 514/252.03; 514/304; 514/317; 514/319; 514/318; 514/255.05; 514/256; 514/326

(57) ABSTRACT

Disclosed are methods for treating Alzheimer's disease, and other diseases, and/or inhibiting beta-secretase enzyme, and/or inhibiting deposition of A beta peptide in a mammal, by use of 3,4-disubstituted piperidinyl compounds of formula (I) wherein the variables R¹, R², R³, R⁴, Q, W, X, Z, m, and n are defined herein.

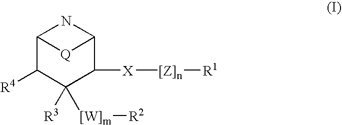

This application claims the benefit of U.S. Provisional Application Nos. 60/278,371, filed Mar. 23, 2001 and 60/308,729 filed Jul. 30, 2001.

### BACKGROUND OF THE INVENTION

1. Field of the Invention

The present invention is the use of known compounds to treat Alzheimer's disease and other similar diseases, and more specifically to compounds that inhibit beta-secretase, an enzyme that cleaves amyloid precursor protein to produce A beta peptide, a major component of the amyloid plaques found in the brains of Alzheimer's sufferers.

2. Background of the Invention

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment know as A beta. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

Smaller numbers of these lesions in a more restricted anatomical distribution are found in the brains of most aged humans who do not have clinical AD. Amyloidogenic plaques and vascular amyloid angiopathy also characterize the brains of individuals with Trisomy 21 (Down's Syndrome), Hereditary Cerebral Hemorrhage with Amyloidosis of the Dutch-Type (HCHWA-D), and other neurodegenerative disorders. Beta-amyloid is a defining feature of AD, now believed to be a causative precursor or factor in the development of disease. Deposition of A beta in areas of the brain responsible for cognitive activities is a major factor in the development of AD. Beta-amyloid plaques are predominantly composed of amyloid beta peptide (A beta, also sometimes designated betaA4). A beta peptide is derived by proteolysis of the amyloid precursor protein (APP) and is comprised of 39-42 amino acids. Several proteases called secretases are involved in the processing of APP.

Cleavage of APP at the N-terminus of the A beta peptide by beta-secretase and at the C-terminus by one or more gamma-secretases constitutes the beta-amyloidogenic pathway, i.e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

An aspartyl protease has been identified as the enzyme responsible for processing of APP at the beta-secretase cleavage site. The beta-secretase enzyme has been disclosed using varied nomenclature, including BACE, Asp, and Memapsin. See, for example, Sindha et al., 1999, Nature 402:537-554 (p501) and published PCT application WO00/17369.

Several lines of evidence indicate that progressive cerebral deposition of beta-amyloid peptide (A beta) plays a seminal role in the pathogenesis of AD and can precede cognitive symptoms by years or decades. See, for example, Selkoe, 1991, Neuron 6:487. Release of A beta from neuronal cells grown in culture and the presence of A beta in cerebrospinal fluid (CSF) of both normal individuals and AD patients has been demonstrated. See, for example, Seubert et al., 1992, Nature 359:325-327.

It has been proposed that A beta peptide accumulates as a result of APP processing by beta-secretase, thus inhibition of this enzyme's activity is desirable for the treatment of AD. In vivo processing of APP at the beta-secretase cleavage site is thought to be a rate-limiting step in A beta production, and is thus a therapeutic target for the treatment of AD. See for example, Sabbagh, M., et al., 1997, Alz. Dis. Rev. 3, 1-19.

BACE1 knockout mice fail to produce A beta, and present a normal phenotype. When crossed with transgenic mice that over express APP, the progeny show reduced amounts of A beta in brain extracts as compared with control animals (Luo et al., 2001 Nature Neuroscience 4:231-232). This evidence further supports the proposal that inhibition of beta-secretase activity and reduction of A beta in the brain provides a therapeutic method for the treatment of AD and other beta amyloid disorders.

At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Compounds that are effective inhibitors of beta-secretase, that inhibit beta-secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

### SUMMARY OF INVENTION

The present invention relates to a method of treating a patient who has, or in preventing a patient from developing, a disease or condition selected from the group consisting of Alzheimer's disease, for helping prevent or delay the onset of Alzheimer's disease, for helping to slow the progression of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I): 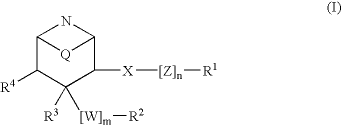
where R¹ is
   (I) aryl, or
   (II) heterocycle;
where R² is
   (I) phenyl,
   (II) naphthyl,
   (III) acenaphthyl,
   (IV) cyclohexyl,
   (V) pyridyl,
   (VI) pyrimidinyl,
   (VII) pyrazinyl,
   (VIII) oxo-pyridinyl,
   (IX) diazinyl,
   (X) triazolyl,
   (XI) thienyl,
   (XII) oxazolyl,
   (XIII) oxadiazolyl,
   (XIV) thiazolyl,
   (XV) pyrrolyl, or
   (XVI) furyl,
   optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
   where L¹, L², L³, L⁴ and L⁵ are independently chosen from:
      (A) a bond,
      (B) C₁₋₈-alkylene,
      (C) C₂₋₈-alkenylene,
      (D) C₂₋₈-alkynylene, or
      (E) are absent,
   where T¹, T², T³, and T⁴ are independently chosen from:
      (A) a bond,
      (B) —CH(OH)—,
      (C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
      (D) —CH(NR⁵R⁶)—, where R⁵ is as defined for R⁶, and where R⁶ is as defined above,
      (E) —CO—,
      (F) —CR⁷R⁸—, where R⁷ and R⁸ together with the C atom to which they are attached form a three, four, five, six, or seven membered ring which can contain one or two O or S atoms or SO or SO₂ groups,
      (G) —O— or —NR⁶—, where R⁶ is as defined above,
      (H) —S(O)₀₋₂—,
      (I) —SO₂NR⁶—, where R⁶ is as defined above,
      (J) —NR⁶SO₂—, where R⁶ is as defined above,
      (K) —CONR⁶—, where R⁶ is as defined above,
      (L) —NR⁶CO—, where R⁶ is as defined above,
      (M) —O—CO—,
      (N) —CO—O—,
      (O) —O—CO—O—,
      (P) —O—CO—NR⁶—, where R⁶ is as defined above,
      (Q) —NR⁶—CO—NR⁶—, where R⁶ are the same or different and are as defined above,
      (R) —NR⁶—CO—O—, where R⁶ is as defined above, or
      (S) are absent
      where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
   where U is:
      (A) hydrogen,
      (B) lower-alkyl,
      (C) cycloalkyl,
      (D) cyano,
      (E) optionally substituted cycloalkyl,
      (F) optionally substituted aryl, or
      (G) optionally substituted heterocyclyl;
where R³ is:
   (I) hydrogen,
   (II) hydroxy,
   (III) lower-alkoxy, or
   (IV) lower-alkenyloxy;
where R⁴ is:
   (I) hydrogen,
   (II) lower-alkyl,
   (III) lower-alkenyl,
   (IV) lower-alkoxy,
   (V) hydroxy-lower-alkyl,
   (VI) lower-alkoxy-lower-alkyl,
   (VII) benzyl,
   (VIII) oxo, or
   (IX) where R³ and R⁴ together are a bond, or R^{4a}-Z¹-X¹—
      where R⁴, is
         (A) H—,
         (B) lower-alkyl-,
         (C) lower-alkenyl-,
         (D) hydroxy-lower-alkyl-,
         (E) polyhydroxy-lower-alkyl-,
         (F) lower-alkyl-O-lower-alkyl-,
         (G) aryl-,
         (H) heterocyclyl-,
         (I) arylalkyl-,
         (J) heterocyclyloxylalkyl-,
         (K) aryloxyalkyl-,
         (L) heterocyclyloxylalkyl-,
         (M) (R⁵R⁶)—N—(CH₂)₁₋₃—, where R¹ and R⁶ are as defined above,
         (N) (R⁵R⁶)—N—, where R⁵ and R⁶ are as defined above,
         (O) lower-alkyl-S(O)₀₋₂—,
         (P) aryl-S(O)₀₋₂—,
         (Q) heterocyclyl-S(O)₀₋₂—,
         (R) HO—SO₃— or a salt thereof,
         (S) H₂N—C(NH)—NH—, or
         (T) NC—,
         where the bonds emanating from (N)-(T) join to a C atom of the adjacent group and this C atom is saturated when the bond emanates from a heteroatom,
      where Z¹ is:
         (A) a bond,
         (B) lower-alkylene-,
         (C) lower-alkenylene-,
         (D) —O—,
         (E) —N(R¹¹)—, where R¹¹ is hydrogen or lower-alkyl,
         (F) —S(O)₀₋₂—,
         (G) —CO—,
         (H) —O—CO—,
         (I) —O—CO—O—,
         (J) —O—CO—N(R¹¹)O, where R¹¹ is as defined above,
         (K) —N(R¹¹)—CO—O—, where R¹¹ is as defined above,
         (L) —CO—N(R¹¹)—, where R¹¹ is as defined above,
         (M) —N(R¹¹)—CO—, where R¹¹ is as defined above,
         (N) —N(R¹¹)—CO—N(R¹¹)—, where R¹¹ are the same or different and are as defined above,
         (O) —CH(OR⁹)—, where R⁹ is hydrogen, lower-alkyl, acyl or arylalkyl, or
         (P) is absent
         where the bonds emanating from (D) and (H)-(O) join to a C atom of the adjacent group and this C atom is saturated when the bond emanates from a heteroatom;
      where X¹ is:
         (A) a bond,
         (B) —O—,
         (C) —N—(R¹¹)—, where R¹¹ is as defined above,
         (D) —S(O)₀₋₂—,
         (E) —(CH₂)₁₋₃—, or
         (F) is absent;
where Q is:
   (I) ethylene, or
   (II) is absent;
where X is:
   (I) a bond,
   (II) —O—,
   (III) —S—,
   (IV) —CH—R¹¹—, where R¹¹ is as defined above,
   (V) —CHOR⁹—, where R⁹ is as defined above,
   (VI) —O—CO,
   (VII) —CO—, or
   (VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
   (I) —O—, or
   (II) —S—;
where Z is:
   (I) lower-alkylene,
   (II) lower-alkenylene,
   (III) hydroxy-lower-alkylidene,
   (IV) —O—,
   (V) —S—,
   (VI) —O-Alk-, where Alk is a lower alkylene
   (VII) —S-Alk-, where Alk is as defined above,
   (VIII) -Alk-O—, where Alk is as defined above, or
   (IX) -Alk-S, where Alk is as defined above;
where n is:
   (I) one, or
   (II) zero or one when X is —O—CO; and
where m is 0 or 1;
with the provisos that
   (I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
   (II) X is —CH—R¹¹— when Z is —O-Alk- or —S-Alk-, and
   (III) Z is lower-alkenylene, -Alk-O— or -Alk-S— when X is a bond,
      or a pharmaceutically acceptable salt thereof.

### DETAILED DESCRIPTION OF THE INVENTION

U.S. Pat. Nos. 6,051,712 and 6,150,526 disclose 3,4-disubstituted piperidinyl compounds of the formula 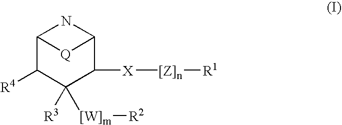
where R¹, R², R³, R⁴, Q, W, X, Z, m and n are as defined above.

These patents disclose how to make the above compounds and how to use them in treating high blood pressure. U.S. Pat. Nos. 6,051,712 and 6,150,526 are incorporated herein by reference in their entirety.

In one aspect, the present invention relates to a method of treating a patient who has, or in preventing a patient from developing, a disease or condition selected from the group consisting of Alzheimer's disease, for helping prevent or delay the onset of Alzheimer's disease, for helping to slow the progression of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I): 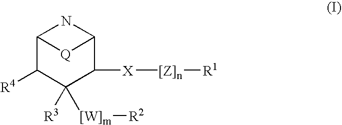
where R¹, R², R³, R⁴, Q, W, X, Z, m and n are as defined above.
   Preferred compounds of formula (I) include the following:
4-[2-[7-[(3R,4R)-4-[4-(3-benzyloxy-propoxy)-phenyl]-piperidin-3-yloxymethyl]-naphthalen-2-yloxy]-ethyl]-morpholine;
(R)-3-[7-[(3R,4R)-4-[4-(3-benzyloxy-propoxy)-phenyl]-piperidin-3-yloxymethyl]-naphthalen-2-yloxy]-propane-1,2-diol;
(S)-3-[7-[(3R,4R)-4-[4-(3-benzyloxy-propoxy)-phenyl]-piperidin-3-yloxymethyl]-naphthalen-2-yloxy]-propane-1,2-diol;
(R)-3-[2-[7-[(3R,4R)-4-[4-(3-benzyloxy-propoxy)-phenyl]-piperidin-3-yloxymethyl]-naphthalen-2-yloxy]-ethoxy]-propane-1,2-diol;
(S)-3-[2-[7-[(3R,4R)-4-[4-(3-benzyloxy-propoxy)-phenyl]-piperidin-3-yloxymethyl]-naphthalen-2-yloxy]-ethoxy]-propane-1,2-diol;
1-[2-[7-[(3R,4R)-4-[4-(3-benzyloxy-propoxy)-phenyl]-piperidin-3-yloxymethyl]-naphthalen-2-yloxy]-ethyl]-4-methyl-piperazine;
1-[(3R,4S-4-[4-(3-benzyloxy-propoxy)-phenyl]-piperidin-3-yl-2-naphthalen-2-yl-ethanone;
(3R,4S,5S)-4-[4-(3-benzyloxy-propoxy)-phenyl]-3-(1,4-dimethoxy-naphthalen-2-ylmethoxy)-piperidin-5-ol;
3R,4R)-4-[4-(3-benzyloxy-propoxy)-phenyl]-3-[7-[(R)-2,3-dihydroxy-propoxymethyl]-naphthalen-2-ylmethoxy]-piperidine;
(3R,4R)-4-[4-(3-benzyloxy-propoxy)-phenyl]-3-[7-[(S)-2,3-dihydroxy-propoxymethyl]-naphthalen-2-ylmethoxy]-piperidine;
(3R,4R)-4-[4-(3-benzyloxy-propoxy)-phenyl]-3-[6-[(R)-2,3-dihydroxy-propoxymethyl]-naphthalen-2-ylmethoxy]-piperidine;
(3R,4R)-4-[4-(3-benzyloxy-propoxy)-phenyl]-3-[6-[(S)-2,3-dihydroxy-propoxymethyl]-naphthalen-2-ylmethoxy]-piperidine;
4-[(3R,4S,5S)-4-[4-(3-benzyloxy-propoxy)-phenyl]-3-(naphthalen-2-ylmethoxy)-piperidin-5-yloxy]-butan-1-ol;
3-[(3R,4S,5S)-4-[4-(3-benzyloxy-propoxy)-phenyl]-3-(naphthalen-2-ylmethoxy)-piperidin-5-yloxy]-propan-1-ol;
1-{2-[(3R,4R,5S)-4-[4-(3-benzyloxy-propoxy)-phenyl]-3-(naphthalen-2-ylmethoxy)-piperidin-5-yloxy]-ethyl}-4-methyl-piperazine;
(3R,4R,5S)-[4-[4(3-benzyloxy-propoxy)-phenyl]-3-(naphthalen-2-ylmethoxy)-piperidin-5-ylmethoxy]-ethyl]-morpholine;
(3R,4S,5S)-4-[4-(3-benzyloxy-propoxy)-phenyl]-3-(4-methoxy-benzyloxy)-piperidin-5-ol;
(3R,4s,5S)-4-[4-(3-benzyloxy-propoxy)-phenyl]-3,5-bis-(4-methoxy-benzyloxy)-piperidine;
(3S,4R,5R)-4-[2-[4-[4-(3-benzyloxy-propoxy)-phenyl]-5-(naphthalen-2-ylmethoxy)-piperidin-3-ylmethoxy]-ethyl]-morpholine;
(3S,4R,5R)-4-[4-(3-benzyloxy-propoxy)-phenyl]-3-methoxymethyl-5-(naphthalen-2-ylmethoxy)-piperidine;
(3S,4R,5R)-4-[4-(3-benzyloxy-propoxy)-phenyl]-5-(naphthalen-2-ylmethoxy)-piperidin-3-ylmethyl[3-(4-methyl-piperazin-1-yl)-propyl]-carbamate;
(3S,4R,5R)-4-[4-[4-(3-benzyloxy-propoxy)-phenyl]-5-(naphthalen-2-ylmethoxy)-piperidin-3-ylmethylsulphanyl]-pyridine;
2-(4-cyclohexyl-butoxy)-5-[(3R,4R)-3-(1,4-dimethoxy-naphthalen-2-ylmethoxy)-piperidin-4-yl]-pyrimidine;
(3′R,4′R)-6-(3-cyclohexyl-propoxy)-3′-(1,4-dimethoxy-naphthalen-2-ylmethoxy)-1′,2′,3′,4′,5′,6′-hexahydro-[3,4′]bipyridine;
(3S,4R,5R)-[4-[4-(3-benzyloxy-propoxy)-phenyl]-5-(naphthalen-2-ylmethoxy)-piperidin-3-yl]-methanol;
(3S,4R,5R)-N-[4-[4-(3-benzyloxy-propoxy)-phenyl]-5-(naphthalen-2-ylmethoxy)-piperidin-3-ylmethyl]-N,N′,N′-trimethyl-ethane-1,2-diamine;
(3S,4R,5R)-[4-[4-(3-benzyloxy-propoxy)-phenyl]-5-(naphthalen-2-ylmethoxy)-piperidin-3-ylmethyl]-diethyl-amine;
1-[(3R,4S,5S)-4-[4-(3-benzyloxy-propoxy)-phenyl]-5-(2-morpholin-4-yl-ethoxymethyl)-piperidin-3-yl]-2-naphthalen-2-yl-ethanone;
(3R,4R)-3-(1,4-dimethoxy-naphthalen-2-ylmethoxy)-4-[4-[3-(2-methoxy-phenoxy)-propoxy]-phenyl]-piperidine;
(3R,4s,5S)-4-[4-(3-benzyloxy-propoxy)-phenyl]-3,5-bis-(3,4,5-trimethoxy-benzyloxy)-piperidine;
(3R,4R,5 S)-4-[4-(3-benzyloxy-propoxy)-phenyl]-3-(naphthalen-2-ylmethoxy)-5-[1,2,4]triazol-1-ylmethyl-piperidine;
(3R,4R)-4-[4-[3-(2-methoxy-benzyloxy)-propoxy]-phenyl]-3-(quinolin-7-ylmethoxy)-piperidine;
2-(7-{(3R,4R)-4-[4-(3-benzyloxy-propoxy)-phenyl]-piperidin-3-yloxymethyl}-naphthalen-2-ylmethoxy)-ethanol;
7-{(3R,4R)-4-[4-(3-benzyloxy-propoxy)-phenyl]-piperidin-3-yloxymethyl}-naphthalen-2-ylmethyl)-dimethyl-amine;
(3R,4R)-3-(4-benzyloxy-naphthalen-2-ylmethoxy)-4-(4-fluoro-phenyl)-piperidine;
(3′R,4′R)-3′-(1,4-dimethoxy-naphthalen-2-ylmethoxy)-6-[3-(2-methoxybenzyloxy)-propoxy]-1′,2′,3′,4′,5′,6′-hexahydro-[3,4′]bipyridine;
(3R,4R)-3-(1,4-dimethoxy-naphthalen-2-ylmethoxy)-4-[4-[3-(2-methoxy-benzyloxy)-propoxy]-phenyl]-piperidine;
(3S,4R,5R)-1-[4-[4-(3-benzyloxy-propoxy)-phenyl]-5-(naphthalen-2-ylmethoxy)-piperidin-3-ylmethyl]-imidazolidin-2-one;
(3R,4R)-4-[4-[3-(2-methoxy-benzyloxy)-propoxy]-phenyl]-3-(2-oxo-1,2-dihydro-quinolin-7-ylmethoxy)-piperidine;
(3R,4R)-3-(isoquinolin-7-ylmethoxy)-4-[4-[3-(2-methoxy-benzyloxy)-propoxy]-phenyl]-piperidine;
(3R,4R)-4-[4-[3-(2-methoxy-benzyloxy)-propoxy]-phenyl]-3-(1,2,3,4-tetrahydro-quinolin-7-ylmethoxy)-piperidine;
1-[2-[7-[(3R,4R)-4-[4-[3-(2-methoxy-benzyloxy)-propoxy]-piperidin-3-yloxymethyl]-naphthalen-2-yloxy]-ethyl]-4-methyl-piperazine;
1-[2-[7-[(3R,4S,5S)-5-hydroxy-4-[4-[3-(2-methoxy-benzyloxy)-propoxy]-piperdin-3-yloxymethyl]-naphthalen-2-yloxy]-ethyl]-4-methyl-piperazine;
(3R,4S,5S)-3-(1,4-dimethoxy-naphthalen-2-ylmethoxy)-4-[4-[3-(2-methoxy-benzyloxy)-propoxy]-phenyl]-piperidin-5-ol;
(3R,4R,5S)-3-(1,4-dimethoxy-naphthalen-2-ylmethoxy)-4-{4-[3-(2-methoxy-benzyloxy)-propoxy]-phenyl}-5-(1H-tetrazol-5-ylmethyl)-piperidine;
(3′S,4′S)-3′-(1,4-dimethoxy-naphthalen-2-ylmethyoxy)-4-[S-(2-methoxy-benzyloxy)-propoxy]-1′,2′,3′,4′,5′,6′-hexahydro-[1,4′]bipyridin-2-one;
(3RS,4RS)-2-[4(3-Naphthalen-2-ylmethoxy-piperidin-4-yl)-phenoxy]-ethylthiophene-2-carboxylate hydrochloride (Example 58-4);
(3RS,4RS)-2-[4(3-Naphthalen-2-ylmethoxy-piperidin-4-yl)-phenoxy]-ethyl 2-chloro-benzoate hydrochloride (Example 54-2);
(3RS,4RS)-2-[4[3[4(2-methoxy-benzyloxy)-naphthalen-2-ylmethoxy]-piperidin-4-yl]-phenoxy]-ethyl benzoate hydrochloride (Example 55-2);
(3RS,4RS)-4-[4(3-Benzyloxy-propoxy)-phenyl]3-(2,3-dihydro-benzo[1,4]dioxin-6-ylmethoxy)-piperidine (Example 86-54);
(3RS,4RS)-3-(Naphthalen-2-ylmethoxy)-4-[4(3-phenyl-[1,2,4]oxadiazol-5-ylmethoxy)-phenyl]-piperidine trifluoroacetate (Example 86-34);
(3RS,4RS)-3-(Naphthalen-2-ylmethoxy)-4-[4(3-phenyl-isoxazol-5-ylmethoxy)-phenyl]-piperidine trifluoroacetate (Example 86-36);
(3RS,4RS)-3-(Naphthalen-2-ylmethoxy)-4-[4(3-phenylsulphanyl-propyl)-phenyl]-piperidine (Example 86-19);
(3RS,4RS)-3-[4[4[2(Benzothiazol-2-ylsulphanyl)-ethyl]-phenyl]-piperidin-3-yloxymethyl]-naphthalen-1-ol (Example 86-23);
(3RS,4RS,5SR)-3-(4-Benzyloxy-naphthalen-2-ylmethoxy)-4-(4-fluoro-phenyl)-5-propyl-piperidine (Example 64);
(3SR,4RS,5RS)-4-(4-Benzyloxymethyl-phenyl)-3-methoxymethyl-5-(naphthalen-2-ylmethoxy)-piperidine (Example 86-60);
(SR)- or (RS)-1-[(3RS,4SR)4-(4-fluoro-phenyl)-piperidin-3-yl]-2-naphthalen-2-ylethyl benzoate hydrochloride (Example 75 b);
(1RS,2RS,3RS,5SR)-2-(4-Benzyloxy-naphthalen-2-ylmethoxy)-3-(4-fluoro-phenyl)-8-aza-bicyclo[3.2.1]octane (Example 84 e);
(3RS,4RS)-4-[4-(3-Benzyloxy-propoxy)-phenyl]-3-(naphthalen-2-ylmethoxy)-piperdine;
4-[2-[7-[(3RS,4RS)-4-[4(3-Benzyloxy-propoxy)-phenyl]piperidin-3-yloxymethyl]-naphthalen-2-yloxy]-ethyl]-morpholine hydrochloride (1:2) (Example 90-07);
Mixture of (RS)- and (SR)-3-[7-[(3RS,4RS)-4-[4(3-benzyloxy-propoxy)-phenyl]-piperidin-3-yloxymethyl]-naphthalen-2-yloxy]-propane-1,2-diol (Example 90-08);
(RS)- and (SR)-3-[2-[7-[(3RS,4RS)-4-[4(3-benzyloxy-propoxy)-phenyl]-piperidin-3-yloxymethyl]-naphthalen-2-yloxy]-ethoxy]-propane-1,2-diol hydrochloride (1:1) (Example 98);
1-[2-[7-[(3RS,4RS)-4-[4-(3-Benzyloxy-propoxy)-phenyl]-piperidin-3-yloxymethyl]-naphthalen-2-yloxy]-ethyl-4-methyl-piperazine hydrochloride (1:3) (Example 90-13);
1-[(3RS,4SR)-4-[4-(3-Benzyloxy-propoxy)-phenyl]-piperidin-3-yl]-2-naphthalen-2-yl-ethanone hydrochloride (1:1) (Example 100);
(3RS,4SR,5SR)-4-[4(3-Benzyloxy-propoxy)-phenyl]-3-(1,4-dimethoxy-naphthalen-2-ylmethoxy)-piperidin-5-ol (Example 109-04);
Mixture of (3RS,4RS)- and (3SR,4SR)-4-[4-(3-benzyloxy-propoxy)-phenyl]-3-[7-[(RS)2,3-dihydroxy-propoxymethyl]naphthalen-2-ylmethoxy]-piperidine (Example 106-02);
Mixture of (3RS,4RS)- and (3SR,4SR)-4-[4-(3-benzyloxy-propoxy)-phenyl]-3-[6-[(RS)-2,3-dihydroxy-propoxymethyl]-naphthalen-2-ylmethoxy]-piperidine (Example 106-01);
4-[(3RS,4SR,5SR)-4-[4(3-Benzyloxy-propoxy)-phenyl]-3-(naphthalen-2-ylmethoxy)-piperidin-5-yloxy]-butan-1-ol (Example 110-08);
3-[(3RS,4SR,5SR)-4-[4(3-Benzyloxy-propoxy)-phenyl]-3-(naphthalen-2-ylmethoxy)-piperidin-5-yloxy]-propan-1-ol (Example 110-07);
1-{2-[(3RS,4RS,5SR)-4-[4-(3-Benzyloxy-propoxy)-phenyl]-3-(naphthalen-2-ylmethoxy)-piperidin-5-yloxy]-ethyl}-4-methyl-piperazine (Example 110-02);
4-{2-[(3RS,4RS,5SR)-4-[4(3-Benzyloxy-propoxy)-phenyl]-3-(naphthalen-2-ylmethoxy)-piperidin-5-yloxy]-ethyl}-morpholine (Example 110-09);
(3RS,4SR,5SR)-4-[4(3-Benzyloxy-propoxy)-pheny]-l3-(4-methoxy-benzyloxy)-piperidin-5-ol (Example 109-28);
(3R,4s,5S)-4-[4(3-Benzyloxy-propoxy)-phenyl]-3,5-bis-(4-methoxy-benzyloxy)-piperidine (Example 109-27);
(3SR,4RS,5RS)-4-[2-[4-[4-(3-Benzyloxy-propoxy)-phenyl]-5-(naphthalen-2-ylmethoxy)-piperidin-3-ylmethoxy]-ethyl]-morpholine (Example 149-04);
(3SR,4RS,5RS)-4-[4-(3-Benzyloxy-propoxy)-phenyl]-3-methoxymethyl-5-(naphthalen-2-ylmethoxy)-piperidine (Example 148);
(3SR,4RS,5RS)-4-[4-(3-Benzyloxy-propoxy)-phenyl]-5-(naphthalen-2-ylmethoxy)-piperidin-3-ylmethyl[3(4-methyl-piperazin-1-yl)-propyl]-carbamate (Example 150-01);
(3SR,4RS,5RS)-4-[4-[4-(3-Benzyloxy-propoxy)-phenyl]-5-(naphthalen-2-ylmethoxy)-piperidin-3-ylmethylsulphanyl]-pyridine (Example 149-02);
2-(4-Cyclohexyl-butoxy)-5-[(3RS,4RS)-3-(1,4-dimethoxy-naphthalen-2-ylmethoxy)-piperidin-4-yl]-pyrimidine (Example 139-03);
(3′RS,4′RS)-6-(3-Cyclohexyl-propoxy)-3′-(1,4-dimethoxy-naphthalen-2-ylmethoxy)-1′,2′,3′,4′,5′,6′-hexahydro-[3,4′]-bipyridine (Example 140-01);
(3SR,4RS,5RS)-[4-[4-(3-Benzyloxy-propoxy)-phenyl]-5-(naphthalen-2-ylmethoxy)-piperidin-3-yl]-methanol hydrochloride (Example 149-01);
(3SR,4RS,5RS)-N-[4-[4-(3-Benzyloxy-propoxy)-phenyl]-5-(naphthalen-2-ylmethoxy)-piperidin-3-ylmethyl]-N,N′,N′-trimethyl-ethane-1,2-diamine (Example 149-06);
(3SR,4RS,5RS)-[4-[4-(3-Benzyloxy-propoxy)-phenyl]-5-(naphthalen-2-ylmethoxy)-piperidin-3-yl]-methyldiethyl-amine (Example 149-05);
1-[(3RS,4SR,5SR)-4-[4-(3-Benzyloxy-propoxy)-phenyl]-5-(2-morpholin-4-yl-ethoxymethyl)-piperidin-3-yl]-2-naphthalen-2-yl-ethanone (Example 101);
(3RS,4RS)-3-(1,4-Dimethoxy-naphthalen-2-ylmethoxy)-4-[4-[3-(2-methoxy-phenoxy)-propoxy]-phenyl]-piperidine (Example 123-27);
(3R,4s,5S)-4-[4-(3-Benzyloxy-propoxy)-phenyl]-3,5-bis-(3,4,5-trimethoxy-benzyloxy)-piperidine (Example 109-29);
(3RS,4RS,5SR)-4-[4-(3-Benzyloxy-propoxy)-phenyl]-3-(naphthalen-2-ylmethoxy)-5-[1,2,4]triazol-1-ylmethyl-piperidine hydrochloride (Example 149-07);
(3RS,4RS)-4-[4-[3-(2-Methoxy-benzyloxy)-propoxy]-phenyl]-3-(quinolin-7-ylmethoxy)-piperidine (Example 120-07);
2-(7-{(3RS,4RS)-4-[4(3-Benzyloxy-propoxy)-phenyl]-piperidin-3-yloxymethyl}-naphthalen-2-ylmethoxy)-ethanol (Example 106-03);
7-{(3RS,4RS)-4-[4-(3-Benzyloxy-propoxy)-phenyl]-piperidin-3-yloxymethyl}-naphthalen-2-ylmethyl)-dimethyl-amine (Example 106-03);
(3R,4R)-3-(4-Benzyloxy-naphthalen-2-ylmethoxy)-4-(4-fluoro-phenyl)-piperidine (Example 154-06);
(3S,4S)-3-(4-Benzyloxy-naphthalen-2-ylmethoxy)-4-(4-fluoro-phenyl)-piperidine (Example 154-07);
(3′RS,4′RS)-3′-(1,4-Dimethoxy-naphthalen-2-ylmethoxy)-6-[3-(2-methoxybenzyloxy)-propoxy]-1′,2′,3′,4′,5′,6′-hexahydro-[3,4′]bipyridine (Example 140-02);
(3RS,4RS)-3-(1,4-Dimethoxy-naphthalen-2-ylmethoxy)-4-[4-[3-(2-methoxy-benzyloxy)-propoxy]-phenyl]-piperidine (Example 123-32);
(3SR,4RS,5RS)-1-[4-[4-(3-Benzyloxy-propoxy)-phenyl]-5-(naphthalen-2-ylmethoxy)-piperidin-3-ylmethyl]-imidazolidin-2-one (Example 149-08);
(3RS,4RS)-4-[4-[3-(2-Methoxy-benzyloxy)-propoxy]-phenyl]-3-(2-oxo-1,2-dihydro-quinolin-7-ylmethoxy)-piperidine (Example 120-10);
(3RS,4RS)-3-(Isoquinolin-7-ylmethoxy)-4-[4-[3-(2-methoxy-benzyloxy)-propoxy]-phenyl]-piperidine (Example 120-11);
(3RS,4RS)-4-[4-[3-(2-Methoxy-benzyloxy)-propoxy]-phenyl]-3-(1,2,3,4-tetrahydro-quinolin-7-ylmethoxy)-piperidine (Example 120-12); and
(3RS,4SR,5SR)-3-(1,4-Dimethoxy-naphthalen-2-ylmethoxy)-4-[4-[3-(2-methoxy-benzyloxy)-propoxy]-phenyl]-piperidin-5-ol (Example 112-11).

In one aspect, this method of treatment can be used where the disease is Alzheimer's disease.

In another aspect, this method of treatment can help prevent or delay the onset of Alzheimer's disease.

In another aspect, this method of treatment can help slow the progression of Alzheimer's disease.

In another aspect, this method of treatment can be used where the disease is mild cognitive impairment.

In another aspect, this method of treatment can be used where the disease is Down's syndrome.

In another aspect, this method of treatment can be used where the disease is Hereditary Cerebral Hemorrhage with Amyloidosis of the Dutch-Type.

In another aspect, this method of treatment can be used where the disease is cerebral amyloid angiopathy.

In another aspect, this method of treatment can be used where the disease is degenerative dementias.

In another aspect, this method of treatment can be used where the disease is diffuse Lewy body type of Alzheimer's disease.

In another aspect, this method of treatment can treat an existing disease, such as those listed above.

In another aspect, this method of treatment can prevent a disease, such as those listed above, from developing or progressing.

The methods of the invention employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

In a preferred aspect, the therapeutically effective amounts for oral administration is from about 1 mg/day to about 100 mg/day; and for parenteral administration from about 5 to about 50 mg daily.

In a more preferred aspect, the therapeutically effective amounts for oral administration is from about 5 mg/day to about 50 mg/day.

The present invention also includes the use of a compound of formula (I), or a pharmaceutically acceptable salt thereof for the manufacture of a medicament for use in treating a patient who has, or in preventing a patient from developing, a disease or condition selected from the group consisting of Alzheimer's disease, for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, diffuse Lewy body type of Alzheimer's disease and who is in need of such treatment.

In one aspect, this use of a compound of formula (I) can be employed where the disease is Alzheimer's disease.

In another aspect, this use of a compound of formula (I) can help prevent or delay the onset of Alzheimer's disease.

In another aspect, this use of a compound of formula (I) can help slow the progression of Alzheimer's disease.

In another aspect, this use of a compound of formula (I) can be employed where the disease is mild cognitive impairment.

In another aspect, this use of a compound of formula (I) can be employed where the disease is Down's syndrome.

In another aspect, this use of a compound of formula (I) can be employed where the disease is Hereditary Cerebral Hemorrhage with Amyloidosis of the Dutch-Type.

In another aspect, this use of a compound of formula (I) can be employed where the disease is cerebral amyloid angiopathy.

In another aspect, this use of a compound of formula (I) can be employed where the disease is degenerative dementias.

In another aspect, this use of a compound of formula (I) can be employed where the disease is diffuse Lewy body type of Alzheimer's disease.

In a preferred aspect, this use of a compound of formula (I) is a pharmaceutically acceptable salt of an acid selected from the group consisting of acids hydrochloric, hydrobromic, hydroiodic, nitric, sulfuric, phosphoric, citric, methanesulfonic, CH₃—(CH₂)ₙ—COOH where n is 0 thru 4, HOOC—(CH₂)ₙ—COOH where n is as defined above, HOOC—CH═CH—COOH, and phenyl-COOH.

The present invention also includes methods for inhibiting beta-secretase activity, for inhibiting cleavage of amyloid precursor protein (APP), in a reaction mixture, at a site between Met596 and Asp597, numbered for the APP-695 amino acid isotype, or at a corresponding site of an isotype or mutant thereof; for inhibiting production of amyloid beta peptide (A beta) in a cell; for inhibiting the production of beta-amyloid plaque in an animal; and for treating or preventing a disease characterized by beta-amyloid deposits in the brain. These methods each include administration of a therapeutically effective amount of a compound of formula (I), or a pharmaceutically acceptable salt thereof.

The present invention also includes a method for inhibiting beta-secretase activity, including exposing said beta-secretase to an effective inhibitory amount of a compound of formula (I), or a pharmaceutically acceptable salt thereof.

In one aspect, this method includes exposing said beta-secretase to said compound in vitro.

In another aspect, this method includes exposing said beta-secretase to said compound in a cell.

In another aspect, this method includes exposing said beta-secretase to said compound in a cell in an animal.

In another aspect, this method includes exposing said beta-secretase to said compound in a human.

The present invention also includes a method for inhibiting cleavage of amyloid precursor protein (APP), in a reaction mixture, at a site between Met596 and Asp597, numbered for the APP-695 amino acid isotype; or at a corresponding site of an isotype or mutant thereof, including exposing said reaction mixture to an effective inhibitory amount of a compound of formula (I), or a pharmaceutically acceptable salt thereof.

In one aspect, this method employs a cleavage site: between Met652 and Asp653, numbered for the APP-751 isotype; between Met 671 and Asp 672, numbered for the APP-770 isotype; between Leu596 and Asp597 of the APP-695 Swedish Mutation; between Leu652 and Asp653 of the APP-751 Swedish Mutation; or between Leu671 and Asp672 of the APP-770 Swedish Mutation.

In another aspect, this method exposes said reaction mixture in vitro.

In another aspect, this method exposes said reaction mixture in a cell.

In another aspect, this method exposes said reaction mixture in an animal cell.

In another aspect, this method exposes said reaction mixture in a human cell.

The present invention also includes a method for inhibiting production of amyloid beta peptide (A beta) in a cell, including administering to said cell an effective inhibitory amount of a compound of formula (I), or a pharmaceutically acceptable salt thereof.

In an embodiment, this method includes administering to an animal.

In an embodiment, this method includes administering to a human.

The present invention also includes a method for inhibiting the production of beta-amyloid plaque in an animal, including administering to said animal an effective inhibitory amount of a compound of formula (I), or a pharmaceutically acceptable salt thereof.

In one aspect, this method includes administering to a human.

The present invention also includes a method for treating or preventing a disease characterized by beta-amyloid deposits in the brain including administering to a patient an effective therapeutic amount of a hydroxyethylene compound of formula (I), or a pharmaceutically acceptable salt thereof.

In one aspect, this method employs a compound at a therapeutic amount in the range of from about 0.1 to about 1000 mg/day.

In another aspect, this method employs a compound at a therapeutic amount in the range of from about 15 to about 1500 mg/day.

In another aspect, this method employs a compound at a therapeutic amount in the range of from about 1 to about 100 mg/day.

In another aspect, this method employs a compound at a therapeutic amount in the range of from about 5 to about 50 mg/day.

In another aspect, this method can be used where said disease is Alzheimer's disease.

In another aspect, this method can be used where said disease is Mild Cognitive Impairment, Down's Syndrome, or Hereditary Cerebral Hemorrhage with Amyloidosis of the Dutch Type.

The present invention also includes a composition including beta-secretase complexed with a compound of formula (I), or a pharmaceutically acceptable salt thereof.

The present invention also includes a method for producing a beta-secretase complex including exposing beta-secretase to a compound of formula (I), or a pharmaceutically acceptable salt thereof, in a reaction mixture under conditions suitable for the production of said complex.

In an embodiment, this method employs exposing in vitro.

In an embodiment, this method employs a reaction mixture that is a cell.

The present invention also includes a component kit including component parts capable of being assembled, in which at least one component part includes a compound of formula (I) enclosed in a container.

In an embodiment, this component kit includes lyophilized compound, and at least one further component part includes a diluent.

The present invention also includes a container kit including a plurality of containers, each container including one or more unit dose of a compound of formula (I):

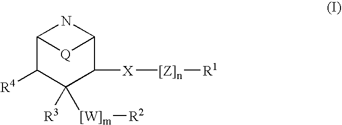

where R¹, R², R³, R⁴, Q, W, X, Z, m and n are as defined above, or a pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The present invention also includes an agent kit including a compound of formula (I), or a pharmaceutically acceptable salt thereof, and one or more therapeutic agents selected from the group consisting of an antioxidant, an anti-inflammatory, a gamma secretase inhibitor, a neurotrophic agent, an acetyl cholinesterase inhibitor, a statin, an A beta peptide, and an anti-A beta antibody.

The present invention provides compounds, compositions, kits, and methods for inhibiting beta-secretase-mediated cleavage of amyloid precursor protein (APP). More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating patients with mild cognitive impairment (MCI), and preventing or delaying the onset of AD in those patients who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, dementia associated with progressive supranuclear palsy, dementia associated with cortical basal degeneration, and diffuse Lewy body type AD.

The compounds of the invention possess beta-secretase inhibitory activity. The inhibitory activities of the compounds of the invention are readily demonstrated, for example, using one or more of the assays described herein or known in the art.

The compounds of formula (I) are amines, and as such form salts when reacted with acids. Pharmaceutically acceptable salts are preferred over the corresponding amines of formula (I) since they frequently produce compounds which are generally more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include acid addition salts of both inorganic and organic acids. The preferred pharmaceutically acceptable salts include salts of the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfuric, tannic, tartaric, teoclic and toluenesulfonic. For other acceptable salts, see Int. J. Pharm., 33, 201-217 (1986) and J. Pharm. Sci., 66(1), 1, (1977).

The present invention provides kits, and methods for inhibiting beta-secretase enzyme activity and A beta peptide production. Inhibition of beta-secretase enzyme activity halts or reduces the production of A beta from APP and reduces or eliminates the formation of beta-amyloid deposits in the brain.

Methods of the Invention

The compounds of the invention, and pharmaceutically acceptable salts thereof, are useful for treating humans or animals suffering from a condition characterized by a pathological form of beta-amyloid peptide, such as beta-amyloid plaques, and for helping to prevent or delay the onset of such a condition. For example, the compounds are useful for treating Alzheimer's disease, for helping prevent or delay the onset of Alzheimer's disease, for treating patients with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type Alzheimer's disease. The compounds and compositions of the invention are particularly useful for treating, preventing, or slowing the progression of Alzheimer's disease. When treating or preventing these diseases, the compounds of the invention can either be used individually or in combination, as is best for the patient.

With regard to these diseases, the term “treating” means that compounds of the invention can be used in humans with existing disease. The compounds of the invention will not necessarily cure the patient who has the disease but will delay or slow the progression or prevent further progression of the disease thereby giving the individual a more useful life span.

The term “preventing” means that that if the compounds of the invention are administered to those who do not now have the disease but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease, such as a known genetic mutation of APP or APP cleavage products in brain tissues or fluids. By delaying the onset of the disease, compounds of the invention have prevented the individual from getting the disease during the period in which the individual would normally have gotten the disease or reduce the rate of development of the disease or some of its effects but for the administration of compounds of the invention up to the time the individual ultimately gets the disease. Preventing also includes administration of the compounds of the invention to those individuals thought to be predisposed to the disease.

In a preferred aspect, the compounds of the invention are useful for slowing the progression of disease symptoms.

In another preferred aspect, the compounds of the invention are useful for preventing the further progression of disease symptoms.

In treating or preventing the above diseases, the compounds of the invention are administered in a therapeutically effective amount. The therapeutically effective amount will vary depending on the particular compound used and the route of administration, as is known to those skilled in the art.

In treating a patient displaying any of the diagnosed above conditions a physician may administer a compound of the invention immediately and continue administration indefinitely, as needed. In treating patients who are not diagnosed as having Alzheimer's disease, but who are believed to be at substantial risk for Alzheimer's disease, the physician should preferably start treatment when the patient first experiences early pre-Alzheimer's symptoms such as, memory or cognitive problems associated with aging. In addition, there are some patients who may be determined to be at risk for developing Alzheimer's through the detection of a genetic marker such as APOE4 or other biological indicators that are predictive for Alzheimer's disease. In these situations, even though the patient does not have symptoms of the disease, administration of the compounds of the invention may be started before symptoms appear, and treatment may be continued indefinitely to prevent or delay the onset of the disease.

Dosage Forms and Amounts

The compounds of the invention can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for intranasal administration is the amount described above for IM administration.

The compounds of the invention can be administered intrathecally. When given by this route the appropriate dosage form can be a parenteral dosage form as is known to those skilled in the art. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg.

The compounds of the invention can be administered by implants as is known to those skilled in the art. When administering a compound of the invention by implant, the therapeutically effective amount is the amount described above for depot administration.

The invention here is the new compounds of the invention and new methods of using the compounds of the invention. Given a particular compound of the invention and a desired dosage form, one skilled in the art would know how to prepare and administer the appropriate dosage form.

The compounds of the invention are used in the same manner, by the same routes of administration, using the same pharmaceutical dosage forms, and at the same dosing schedule as described above, for preventing disease or treating patients with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The compounds of the invention can be used with each other or with other agents used to treat or prevent the conditions listed above. Such agents include gamma-secretase inhibitors, anti-amyloid vaccines and pharmaceutical agents such as donepezil hydrochloride (ARICEPT Tablets), tacrine hydrochloride (COGNEX Capsules) or other acetylcholine esterase inhibitors and with direct or indirect neurotropic agents of the future.

In addition, the compounds of the invention can also be used with inhibitors of P-glycoproten (P-gp). The use of P-gp inhibitors is known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. The important thing is that the blood level of the P-gp inhibitor be such that it exerts its effect in inhibiting P-gp from decreasing brain blood levels of the compounds of the invention. To that end the P-gp inhibitor and the compounds of the invention can be administered at the same time, by the same or different route of administration, or at different times. The important thing is not the time of administration but having an effective blood level of the P-gp inhibitor.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 and other steroids. It is to be understood that additional agents will be found that do the same function and are also considered to be useful.

The P-gp inhibitors can be administered orally, parenterally, (IV, IM, IM-depo, SQ, SQ-depo), topically, sublingually, rectally, intranasally, intrathecally and by implant.

The therapeutically effective amount of the P-gp inhibitors is from about 0.1 to about 300 mg/kg/day, preferably about 0.1 to about 150 mg/kg daily. It is understood that while a patient may be started on one dose, that dose may have to be varied over time as the patient's condition changes.

When administered orally, the P-gp inhibitors can be administered in usual dosage forms for oral administration as is known to those skilled in the art. These dosage forms include the usual solid unit dosage forms of tablets and capsules as well as liquid dosage forms such as solutions, suspensions and elixirs. When the solid dosage forms are used, it is preferred that they be of the sustained release type so that the P-gp inhibitors need to be administered only once or twice daily. The oral dosage forms are administered to the patient one thru four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that what ever dosage form is used, that it be designed so as to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered IV, IM, depo-IM, SQ or depo-SQ. The P-gp inhibitors can be given sublingually. When given sublingually, the P-gp inhibitors should be given one thru four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the path is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art. The P-gp inhibitors can be administered rectally by suppository as is known to those skilled in the art.

The P-gp inhibitors can be administered by implants as is known to those skilled in the art.

There is nothing novel about the route of administration nor the dosage forms for administering the P-gp inhibitors. Given a particular P-gp inhibitor, and a desired dosage form, one skilled in the art would know how to prepare the appropriate dosage form for the P-gp inhibitor.

The compounds employed in the methods of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

It should be apparent to one skilled in the art that the exact dosage and frequency of administration will depend on the particular compounds employed in the methods of the invention administered, the particular condition being treated, the severity of the condition being treated, the age, weight, general physical condition of the particular patient, and other medication the individual may be taking as is well known to administering physicians who are skilled in this art.

Inhibition of APP Cleavage

The compounds of the invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the Examples below.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Beta-Secretase

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et al., 1999, Mol. Cell. Neurosci. 14:419-427; Vassar et al., 1999, Science 286:735-741; Yan et al., 1999, Nature 402:533-537; Sinha et al., 1999, Nature 40:537-540; and Lin et al., 2000, PNAS USA 97:1456-1460). Synthetic forms of the enzyme have also been described (WO98/22597 and WO00/17369). Beta-secretase can be extracted and purified from human brain tissue and can be produced in cells, for example mammalian cells expressing recombinant enzyme.

Useful inhibitory compounds are effective to inhibit 50% of beta-secretase enzymatic activity at a concentration of less than 50 micromolar, preferably at a concentration of 10 micromolar or less, more preferably 1 micromolar or less, and most preferably 10 nanomolar or less.

APP Substrate

Assays that demonstrate inhibition of beta-secretase-mediated cleavage of APP can utilize any of the known forms of APP, including the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the beta-secretase cleavage site, wild type (WT) or mutated form, e.g., SW, as described, for example, in U.S. Pat. No. 5,942,400 and WO00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety may be an antigenic epitope for antibody binding, a label or other detection moiety, a binding substrate, and the like.

Antibodies

Products characteristic of APP cleavage can be measured by immunoassay using various antibodies, as described, for example, in Pirttila et al., 1999, Neuro. Lett. 249:21-4, and in U.S. Pat. No. 5,612,486. Useful antibodies to detect A beta include, for example, the monoclonal antibody 6E10 (Senetek, St. Louis, Mo.) that specifically recognizes an epitope on amino acids 1-16 of the A beta peptide; antibodies 162 and 164 (New York State Institute for Basic Research, Staten Island, N.Y.) that are specific for human A beta 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

Cell Free Assays

Exemplary assays that can be used to demonstrate the inhibitory activity of the compounds of the invention are described, for example, in WO00/17369, WO 00/03819, and U.S. Pat. Nos. 5,942,400 and 5,744,346. Such assays can be performed in cell-free incubations or in cellular incubations using cells expressing a beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example: approximately 200 nanomolar to 10 micromolar substrate, approximately 10 to 200 picomolar enzyme, and approximately 0.1 nanomolar to 10 micromolar inhibitor compound, in aqueous solution, at an approximate pH of 4-7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

Cellular Assay

Numerous cell-based assays can be used to analyze beta-secretase activity and/or processing of APP to release A beta. Contact of an APP substrate with a beta-secretase enzyme within the cell and in the presence or absence of a compound inhibitor of the invention can be used to demonstrate beta-secretase inhibitory activity of the compound. Preferably, assay in the presence of a useful inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a useful means to assay inhibitory activities of the compounds of the invention. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by EIA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

In Vivo Assays: Animal Models

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015, and 5,811,633, and in Ganes et al., 1995, Nature 373:523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plaques is preferred.

On contacting an APP substrate with a beta-secretase enzyme in the presence of an inhibitory compound of the invention and under conditions sufficient to permit enzymatic mediated cleavage of APP and/or release of A beta from the substrate, the compounds of the invention are effective to reduce beta-secretase-mediated cleavage of APP at the beta-secretase cleavage site and/or effective to reduce released amounts of A beta. Where such contacting is the administration of the inhibitory compounds of the invention to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a patient at risk for the disease.

Unless defined otherwise, all scientific and technical terms used herein have the same meaning as commonly understood by one of skill in the art to which this invention belongs. All patents and publications referred to herein are hereby incorporated by reference for all purposes.

Definitions and Conventions

The definitions and explanations below are for terms as used throughout this entire document including both the specification and claims.

Conventions for Formulas and Definitions of Variables

The chemical formulas representing various compounds or molecular fragments in the specification and claims may contain variable substituents in addition to expressly defined structural features. These variable substituents are identified by a letter or a letter followed by a numerical subscript, for example, “Z₁” or “Rᵢ” where “i” is an integer. These variable substituents are either monovalent or bivalent, that is, they represent a group attached to the formula by one or two chemical bonds. For example, a group Z₁ would represent a bivalent variable if attached to the formula CH₃—C(=Z₁)H. Groups Rᵢ and Rⱼ would represent monovalent variable substituents if attached to the formula CH₃—CH₂—C(Rᵢ)(Rⱼ)H₂. When chemical formulas are drawn in a linear fashion, such as those above, variable substituents contained in parentheses are bonded to the atom immediately to the left of the variable substituent enclosed in parentheses. When two or more consecutive variable substituents are enclosed in parentheses, each of the consecutive variable substituents is bonded to the immediately preceding atom to the left which is not enclosed in parentheses. Thus, in the formula above, both Rᵢ and Rⱼ are bonded to the preceding carbon atom. Also, for any molecule with an established system of carbon atom numbering, such as steroids, these carbon atoms are designated as Cᵢ, where “i” is the integer corresponding to the carbon atom number. For example, C₆ represents the 6 position or carbon atom number in the steroid nucleus as traditionally designated by those skilled in the art of steroid chemistry. Likewise the term “R₆” represents a variable substituent (either monovalent or bivalent) at the C₆ position.

Chemical formulas or portions thereof drawn in a linear fashion represent atoms in a linear chain. The symbol “—” in general represents a bond between two atoms in the chain. Thus CH₃—O—CH₂—CH(Rᵢ)—CH₃ represents a 2-substituted-1-methoxypropane compound. In a similar fashion, the symbol “═” represents a double bond, e.g., CH₂═C(Rᵢ)—O—CH₃, and the symbol “≡” represents a triple bond, e.g., HC≡C—CH(Rᵢ)—CH₂—CH₃. Carbonyl groups are represented in either one of two ways: —CO— or —C(═O)—, with the former being preferred for simplicity.

Chemical formulas of cyclic (ring) compounds or molecular fragments can be represented in a linear fashion. Thus, the compound 4-chloro-2-methylpyridine can be represented in linear fashion by N*═C(CH₃)—CH═CCl—CH═C*H with the convention that the atoms marked with an asterisk (*) are bonded to each other resulting in the formation of a ring. Likewise, the cyclic molecular fragment, 4-(ethyl)-1-piperazinyl can be represented by —N*-(CH₂)₂—N(C₂H₅)—CH₂—C*H₂.

A rigid cyclic (ring) structure for any compounds herein defines an orientation with respect to the plane of the ring for substituents attached to each carbon atom of the rigid cyclic compound. For saturated compounds which have two substituents attached to a carbon atom which is part of a cyclic system, —C(X₁)(X₂)— the two substituents may be in either an axial or equatorial position relative to the ring and may change between axial/equatorial. However, the position of the two substituents relative to the ring and each other remains fixed. While either substituent at times may lie in the plane of the ring (equatorial) rather than above or below the plane (axial), one substituent is always above the other. In chemical structural formulas depicting such compounds, a substituent (X₁) which is “below” another substituent (X₂) will be identified as being in the alpha configuration and is identified by a broken, dashed or dotted line attachment to the carbon atom, i.e., by the symbol “ - - - ” or “ . . . ”. The corresponding substituent attached “above” (X₂) the other (X₁) is identified as being in the beta configuration and is indicated by an unbroken line attachment to the carbon atom.

When a variable substituent is bivalent, the valences may be taken together or separately or both in the definition of the variable. For example, a variable Rᵢ attached to a carbon atom as —C(═Rᵢ)— might be bivalent and be defined as oxo or keto (thus forming a carbonyl group (—CO—) or as two separately attached monovalent variable substituents alpha-Rᵢ₋ⱼ and beta-Rᵢ₋ₖ. When a bivalent variable, Rᵢ, is defined to consist of two monovalent variable substituents, the convention used to define the bivalent variable is of the form “alpha-Rᵢ₋ⱼ:beta-Rᵢ₋ₖ” or some variant thereof. In such a case both alpha-Rᵢ₋ⱼ and beta-Rᵢ₋ₖ are attached to the carbon atom to give —C(alpha-Rᵢ₋ⱼ)(beta-Rᵢ₋ⱼ)—. For example, when the bivalent variable R₆, —C(═R₆)— is defined to consist of two monovalent variable substituents, the two monovalent variable substituents are alpha-R₆₋₁:beta-R₆₋₂, . . . alpha-R₆₋₉:beta-R₆₋₁₀, etc, giving —C(alpha-R₆₋₁)(beta-R₆₋₂)—, . . . —C(alpha-R₆₋₉)(beta-R₆₋₁₀)—, etc. Likewise, for the bivalent variable R₁₁, —C(═R₁₁)—, two monovalent variable substituents are alpha-R₁₁₋₁:beta-R₁₁₋₂. For a ring substituent for which separate alpha and beta orientations do not exist (e.g. due to the presence of a carbon carbon double bond in the ring), and for a substituent bonded to a carbon atom which is not part of a ring the above convention is still used, but the alpha and beta designations are omitted.

Just as a bivalent variable may be defined as two separate monovalent variable substituents, two separate monovalent variable substituents may be defined to be taken together to form a bivalent variable. For example, in the formula —C₁(Rᵢ)H—C₂(Rⱼ)H— (C₁ and C₂ define arbitrarily a first and second carbon atom, respectively) Rᵢ and Rⱼ may be defined to be taken together to form (1) a second bond between C₁ and C₂ or (2) a bivalent group such as oxa (—O—) and the formula thereby describes an epoxide. When Rᵢ and Rⱼ are taken together to form a more complex entity, such as the group —X—Y—, then the orientation of the entity is such that C₁ in the above formula is bonded to X and C₂ is bonded to Y. Thus, by convention the designation “ . . . Rᵢ and Rⱼ are taken together to form —CH₂—CH₂—O—CO— . . . ” means a lactone in which the carbonyl is bonded to C₂. However, when designated “ . . . Rⱼ and Rᵢ are taken together to form —CO—O—CH₂—CH₂— the convention means a lactone in which the carbonyl is bonded to C₁.

The carbon atom content of variable substituents is indicated in one of two ways. The first method uses a prefix to the entire name of the variable such as “C₁-C₄”, where both “1” and “4” are integers representing the minimum and maximum number of carbon atoms in the variable. The prefix is separated from the variable by a space. For example, “C₁-C₄ alkyl” represents alkyl of 1 through 4 carbon atoms, (including isomeric forms thereof unless an express indication to the contrary is given). Whenever this single prefix is given, the prefix indicates the entire carbon atom content of the variable being defined. Thus C₂-C₄ alkoxy-carbonyl describes a group CH₃—(CH₂)ₙ—O—CO— where n is zero, one or two. By the second method the carbon atom content of only each portion of the definition is indicated separately by enclosing the “Cᵢ-Cⱼ” designation in parentheses and placing it immediately (no intervening space) before the portion of the definition being defined. By this optional convention (C₁-C₃)alkoxycarbonyl has the same meaning as C₂-C₄ alkoxycarbonyl because the “C₁-C₃” refers only to the carbon atom content of the alkoxy group. Similarly while both C₂-C₆ alkoxyalkyl and (C₁-C₃)alkoxy(C₁-C₃)alkyl define alkoxyalkyl groups containing from 2 to 6 carbon atoms, the two definitions differ since the former definition allows either the alkoxy or alkyl portion alone to contain 4 or 5 carbon atoms while the latter definition limits either of these groups to 3 carbon atoms.

When the claims contain a fairly complex (cyclic) substituent, at the end of the phrase naming/designating that particular substituent will be a notation in (parentheses) which will correspond to the same name/designation in one of the CHARTS which will also set forth the chemical structural formula of that particular substituent.

Definitions

The term “lower” used here denotes groups with 1-6, preferably 1-4, C atoms. Examples of lower alkyl and alkoxy groups are methyl, ethyl, n-propyl, isopropyl, n-butyl, isobutyl, sec-butyl, tert-butyl, pentyl, hexyl and respectively, methoxy, ethoxy, propoxy, isopropoxy, butoxy, isobutoxy, sec.-butoxy and tert-butoxy. Lower-alkylenedioxy groups are preferably methylenedioxy, ethylenedioxy and propylenedioxy. Acetyl, propionyl and butyryl are examples of lower-alkanoyl groups.

Cycloalkyl signifies a saturated, cyclic hydrocarbon group with 3-6 carbon atoms, including for example cyclopropyl, cyclobutyl, cyclopentyl and cyclohexyl. C₁₋₈-Alkylene groups include, for example methylene, ethylene, propylene, 2-methyl-propylene, tetra-, penta- and hexamethylene; C₂₋₈-alkenylene groups include for example vinylene and propenylene; C₂₋₈-alkynylene groups include for example ethynylene. Acyl groups are alkanoyl groups, preferably lower-alkanoyl groups, or aroyl groups such as benzoyl. Aryl denotes mono-nuclear or poly-nuclear aromatic groups which can carry one or more substituents, such as, for example, phenyl, substituted phenyl, naphthyl, substituted naphthyl, tetrahydronaphthyl or substituted tetrahydronaphthyl, tetrahydronaphthyl or substituted tetrahydronaphthyl. Examples of substitutents on such aryl groups include for example lower-alkyl, trifluoromethyl, nitro, amino, lower-alkenyl, lower-alkoxy, lower-alkylcarbonyloxy, hydroxy, halogen, cyano, carbamoyl and lower-alkylenedioxy, as well as optionally halo-, lower-alkyl-, lower-alkoxy- or dihydroxy-lower-alkylaminocarbonyl-substituted phenyl, phenoxy, phenylthio, phenyl-lower-alkyl or phenyl-lower-alkoxy, Further examples of substituents on aryl groups include lower-alkoxycarbonylphenyl, hydroxy-lower-alkylphenyl, benzyloxy, pyridylcarbonylamino-lower-alkyl, lower-alkenyloxy, lower-alkoxy-lower-alkoxy, methoxybenzyloxy, hydroxybenzyloxy, phenethyloxy, methylenedioxybenzyloxy, dioxolanyl-lower-alkoxy, cyclopropyl-lower-alkoxy, hydroxy-lower-alkoxy, carbamoyloxy-lower-alkoxy, pyridyl-carbamoyloxy-lower-alkoxy, benzoyloxy-lower-alkoxy; as well as optionally halo-, lower-alkyl-, lower-alkoxy- or dihydroxy-lower-alkylaminocarbonyl-subsituted pyridyl, pyridyloxy, pyridylthio, pyridylamino, pyridyl-lower-alkyl, pyridyl-lower-alkoxy, pyrimidinyl, pyrimidinyloxy, pyrimidinylthio, pyridinylamino, pyrimidinyl-lower-alkyl, pyrimidinyl-lower-alkoxy, thienyl, thienyl-lower-alkyl, thienyl-lower-alkoxy, furyl, furyl-lower-alkyl and furyl-lower-alkoxy.

The term heterocycle or heterocyclyl denotes monocyclic or bicyclic, saturated and unsaturated heterocyclic groups with 1 to 4 nitrogen atoms and/or 1 or 2 sulfur or oxygen atoms, which can be mono- or multiply-substituted, especially by (in the case of unsaturated heterocyclyl groups) alkyl, hydroxy, alkoxy, nitro or halogen or by substituents as defined above for aryl groups or (in the case of saturated heterocyclyl groups) by alkyl or alkoxy. Examples of heterocyclyl groups include pyridyl, thienyl, pyrazinyl, triazolyl, imidazolyl, benzthiazolyl, furyl, pyrimidinyl, morpholinyl, quinazolinyl, quinolyl, quinoxalinyl, isoquinolyl, benzo[b]thienyl, isobenzofuranyl, benzimidazolyl, 2-oxo-benzimidazolyl or thiazolyl. Examples of substituted heterocyclyl groups include nitrobenzthiazolyl, phenyl-tetrazolyl, phenyl-oxazolyl. Examples of saturated heterocyclyl groups include dioxolanyl, dioxanyl, dithiolanyl, dithianyl, pyrrolidinyl, piperidinyl, piperazinyl, 4-methylpiperazinyl, morpholinyl, thiomorpholinyl, 2-hydroxymetthylpyrrolidinyl, 3-hydroxypyrrolidinyl, 3,4-dihydroxypyrrolidinyl, 4-hydroxypiperidinyl, 4-oxopiperidinyl, 3,5-dimethylomorpholinyl, 4,4-dioxothiomorpholinyl, 4-oxothiomorpholinyl, 2,6-dimethylmorpholinyl, 2-oxo-imidazolidinyl, 2-oxo-oxazolidinyl, 2-oxo-pyrrolidinyl, 2-oxo[1,3]oxazinyl, 2-oxo-tetrahydro-pyrimidinyl and the like.

In the case of R¹, R^{4a} and R⁹ the aryl, aroyl and heterocyclyl groups can be additionally substituted by heterocyclylalkyl, heterocyclylalkoxy or hetherocyclylalkoxyalkyl, such as, for example, piperidinoalkyl, piperidinoalkoxy, piperidinoalkoxyalkyl, morpholinoalkyl, morpholinoalkoxy, morpholinoalkoxyalkyl, piperazinoalkyl, piperazinoalkoxy, piperazinoalkoxyalkyl or N-methylpiperazinoalkyl, N-methylpiperazinoalkoxy, N-methylpiperazinoalkoxyalkyl, as well as alkylaminolkyl, alkylamino-alkoxy, alkylamino-alkoxyalkyl, mono- and polyhydroxy-alkyl, -alkoxy, -alkoxyalkyl and -alkoxyalkoxy, carbamoylalkyloxy, lower-alkoxy, amino-lower-alkoxy, hydroxy-lower-alkoxy or by the —O—CH₂CH(OH)CH₂NR^{x} group, in which NR^{x} is a mono- or di-lower-alkylamino, piperidino, morpholino, piperazino or N-methylpiperazino group. Examples of 5- and 6-membered heterocyclic rings denoted by NR⁵R⁶ include pyrrolidinyl, piperidinyl, piperazinyl, morpholinyl, thiomorpholinyl, 2-hydroxymethylpyrrolidinyl, 3-hydroxypyrrolidinyl; 3,4-dihydroxypyrrolidinyl, 4-hydroxypiperidinyl, 4-oxopiperidinyl, 3,5-dimethylmorpholinyl, 4,4-dioxothiomorpholinyl, 4-oxothiomorpholinyl, 2,6-dimethylmorpholinyl, 2-oxo-imidazolidinyl, 2-oxo-oxazolidinyl, 2-oxo-pyrrolidinyl, 2-oxo-[1,3]oxazinyl, 2-oxo-tetrahydro-pyrimidinyl and the like. Cyclopentyl, cyclohexyl, cycloheptyl, 1,3-dioxolanyl, 1,3-dioxanyl, 1,3-dithiolanyl and 1,3-dithianyl are examples of 3-7 membered rings denoted by CR⁷R⁸. The term polyhydroxy-alkyl denotes C₁-C₇-alkyl groups which can be substituted by 2-6 hydroxy groups, including for example glyceryl, arabityl, sorbityl etc.

All temperatures are in degrees Celsius.

HPLC refers to high pressure liquid chromatography.

Saline refers to an aqueous saturated sodium chloride solution.

Chromatography (column and flash chromatography) refers to purification/separation of compounds expressed as (support, eluent). It is understood that the appropriate fractions are pooled and concentrated to give the desired compound(s).

APP, amyloid precursor protein, is defined as any APP polypeptide, including APP variants, mutations, and isoforms, for example, as disclosed in U.S. Pat. No. 5,766,846.

A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Beta-secretase (BACE1, Asp2, Memapsin 2) is an aspartyl protease that mediates cleavage of APP at the amino-terminal edge of A beta. Human beta-secretase is described, for example, in WO00/17369.

Pharmaceutically acceptable refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

A therapeutically effective amount is defined as an amount effective to reduce or lessen at least one symptom of the disease being treated or to reduce or delay onset of one or more clinical markers or symptoms of the disease.

It should be noted that, as used in this specification and the appended claims, the singular forms “a”, “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Unless defined otherwise, all scientific and technical terms used herein have the same meaning as commonly understood by one of skill in the art to which this invention belongs.

All patents and publications referred to herein are hereby incorporated by reference for all purposes.

The present invention may be better understood with reference to the following examples. These examples are intended to be representative of specific embodiments of the invention, and are not intended as limiting the scope of the invention.

### EXAMPLES

### Example A

Enzyme Inhibition Assay

The compounds of the invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of maltose binding protein (MBP) and the carboxy terminal 125 amino acids of APP-SW, the Swedish mutation. The beta-secretase enzyme is derived from human brain tissue as described in Sinha et al, 1999, Nature 40:537-540) or recombinantly produced as the full-length enzyme (amino acids 1-501), and can be prepared, for example, from 293 cells expressing the recombinant cDNA, as described in WO00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti-SW192 reporter antibody, and further incubation with streptavidin/alkaline phosphatase. In the assay, cleavage of the intact MBP-C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu 596 at the substrate's APP-SW 751 mutation site.

Specific Assay Procedure:

Compounds are diluted in a 1:1 dilution series to a six-point concentration curve (two wells per concentration) in one 96-plate row per compound tested. Each of the test compounds is prepared in DMSO to make up a 10 millimolar stock solution. The stock solution is serially diluted in DMSO to obtain a final compound concentration of 200 micromolar at the high point of a 6-point dilution curve. Ten (10) microliters of each dilution is added to each of two wells on row C of a corresponding V-bottom plate to which 190 microliters of 52 millimolar NaOAc, 7.9% DMSO, pH 4.5 are pre-added. The NaOAc diluted compound plate is spun down to pellet precipitant and 20 microliters/well is transferred to a corresponding flat-bottom plate to which 30 microliters of ice-cold enzyme-substrate mixture (2.5 microliters MBP-C125SW substrate, 0.03 microliters enzyme and 24.5 microliters ice cold 0.09% TX100 per 30 microliters) is added. The final reaction mixture of 200 micromolar compound at the highest curve point is in 5% DMSO, 20 millimolar NaOAc, 0.06% TX100, at pH 4.5.

Warming the plates to 37 degrees C. starts the enzyme reaction. After 90 minutes at 37 degrees C., 200 microliters/well cold specimen diluent is added to stop the reaction and 20 microliters/well was transferred to a corresponding anti-MBP antibody coated ELISA plate for capture, containing 80 microliters/well specimen diluent. This reaction is incubated overnight at 4 degrees C. and the ELISA is developed the next day after a 2 hour incubation with anti-192SW antibody, followed by Streptavidin-AP conjugate and fluorescent substrate. The signal is read on a fluorescent plate reader.

Relative compound inhibition potency is determined by calculating the concentration of compound that showed a fifty percent reduction in detected signal (IC₅₀) compared to the enzyme reaction signal in the control wells with no added compound. In this assay, the compounds of the invention exhibited an IC₅₀ of less than 50 micromolar.

### Example B

Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds of the invention. Useful substrates include the following:

| | |
|---|---|
| Biotin-SEVNL-DAEFRC[Oregon green]KK | [SEQ ID NO: 1] |
| Biotin-SEVKM-DAEFRC[Oregon green]KK | [SEQ ID NO: 2] |
| Biotin-GLNIKTEEISEISY-EVEFRC[Oregon | [SEQ ID NO: 3] |
| green]KK | |
| Biotin-ADRGLTTRPGSGLTNIKTEEISEVNL- | [SEQ ID NO: 4] |
| DAEFC[Oregon green]KK | |
| Biotin-FVNQHLC_{OX}GSHLVEALY- | [SEQ ID NO: 5] |
| LVC_{OX}GERGFFYTPKAC[Oregon green]KK | |

The enzyme (0.1 nanomolar) and test compounds (0.001-100 micromolar) are incubated in pre-blocked, low affinity, black plates (384 well) at 37 degrees for 30 minutes. The reaction is initiated by addition of 150 millimolar substrate to a final volume of 30 microliter per well. The final assay conditions are: 0.001-100 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin. After incubation with streptavidin at room temperature for 15 minutes, fluorescence polarization is measured, for example, using a LJL Acqurest (Ex485 nm/Em530 mm). The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate. In this assay, compounds of the invention exhibited an IC₅₀ of less than 50 micromolar.

### Example C

Beta-Secretase Inhibition: P26-P4′SW Assay

Synthetic substrates containing the beta-secretase cleavage site of APP are used to assay beta-secretase activity, using the methods described, for example, in published PCT application WO00/47618. The P26-P4′SW substrate is a peptide of the sequence:

[SEQ ID NO: 6]

(biotin)CGGADRGLTTRPGSGLTNIKTEEISEVNLDAEF

The P26-P1 standard has the sequence:

[SEQ ID NO: 7]

(biotin)CGGADRGLTTRPGSGLTNIKTEEISEVNL.

Briefly, the biotin-coupled synthetic substrates are incubated at a concentration of from about 0 to about 200 micromolar in this assay. When testing inhibitory compounds, a substrate concentration of about 1.0 micromolar is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 to 2000 picomolar, after dilution.

The reaction mixture also includes 20 millimolar sodium acetate, pH 4.5, 0.06% Triton X100, and is incubated at 37 degrees C. for about 1 to 3 hours. Samples are then diluted in assay buffer (for example, 145.4 nanomolar sodium chloride, 9.51 millimolar sodium phosphate, 7.7 millimolar sodium azide, 0.05% Triton X405, 6 g/liter bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 hours at 4 degrees C. After washing in TTBS buffer (150 millimolar sodium chloride, 25 millimolar Tris, 0.05% Tween 20, pH 7.5), the samples are incubated with streptavidin-AP according to the manufacturer's instructions. After a one hour incubation at room temperature, the samples are washed in TTBS and incubated with fluorescent substrate solution A (31.2 g/liter 2-amino-2-methyl-1-propanol, 30 mg/liter, pH 9.5). Reaction with streptavidin-alkaline phosphate permits detection by fluorescence. Compounds that are effective inhibitors of beta-secretase activity demonstrate reduced cleavage of the substrate as compared to a control.

### Example D

Assays Using Synthetic Oligopeptide-Substrates

Synthetic oligopeptides are prepared that incorporate the known cleavage site of beta-secretase, and optionally detectable tags, such as fluorescent or chromogenic moieties. Examples of such peptides, as well as their production and detection methods are described in U.S. Pat. No. 5,942,400, herein incorporated by reference. Cleavage products can be detected using high performance liquid chromatography, or fluorescent or chromogenic detection methods appropriate to the peptide to be detected, according to methods well known in the art.

By way of example, one such peptide has the sequence (biotin)-SEVNL-DAEF [SEQ ID NO: 8], and the cleavage site is between residues 5 and 6. Another preferred substrate has the sequence ADRGLTTRPGSGLTNIKTEEISEVNL-DAEF [SEQ ID NO: 9], and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of the compound inhibitor to control results provides a measure of the compound's inhibitory activity.

### Example E

Inhibition of Beta-Secretase Activity—Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met52 to Asn651Leu652 (numbered for APP751), commonly called the Swedish mutation and shown to overproduce A beta (Citron et al., 1992, Nature 360:672-674), as described in U.S. Pat. No. 5,604,102.

The cells are incubated in the presence/absence of the inhibitory compound (diluted in DMSO) at the desired concentration, generally up to 10 micrograms/ml. At the end of the treatment period, conditioned media is analyzed for beta-secretase activity, for example, by analysis of cleavage fragments. A beta can be analyzed by immunoassay, using specific detection antibodies. The enzymatic activity is measured in the presence and absence of the compound inhibitors to demonstrate specific inhibition of beta-secretase mediated cleavage of APP substrate.

### Example F

Inhibition of Beta-Secretase in Animal Models of AD

Various animal models can be used to screen for inhibition of beta-secretase activity. Examples of animal models useful in the invention include, but are not limited to, mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

PDAPP mice, prepared as described in Games et al., 1995, Nature 373:523-527 are useful to analyze in vivo suppression of A beta release in the presence of putative inhibitory compounds. As described in U.S. Pat. No. 6,191,166, 4 month old PDAPP mice are administered compound formulated in vehicle, such as corn oil. The mice are dosed with compound (1-30 mg/ml; preferably 1-10 mg/ml). After time, e.g., 3-10 hours, the animals are sacrificed, and brains removed for analysis.

Transgenic animals are administered an amount of the compound inhibitor formulated in a carrier suitable for the chosen mode of administration. Control animals are untreated, treated with vehicle, or treated with an inactive compound. Administration can be acute, i.e., single dose or multiple doses in one day, or can be chronic, i.e., dosing is repeated daily for a period of days. Beginning at time 0, brain tissue or cerebral fluid is obtained from selected animals and analyzed for the presence of APP cleavage peptides, including A beta, for example, by immunoassay using specific antibodies for A beta detection. At the end of the test period, animals are sacrificed and brain tissue or cerebral fluid is analyzed for the presence of A beta and/or beta-amyloid plaques. The tissue is also analyzed for necrosis.

Animals administered the compound inhibitors of the invention are expected to demonstrate reduced A beta in brain tissues or cerebral fluids and reduced beta amyloid plaques in brain tissue, as compared with non-treated controls.

### Example G

Inhibition of A Beta Production in Human Patients

Patients suffering from Alzheimer's Disease (AD) demonstrate an increased amount of A beta in the brain. AD patients are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated patients.

### Example H

Prevention of A Beta Production in Patients at Risk for AD

Patients predisposed or at risk for developing AD are identified either by recognition of a familial inheritance pattern, for example, presence of the Swedish Mutation, and/or by monitoring diagnostic parameters. Patients identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated patients.

Abbreviations:

The following abbreviations are used throughout the following examples:
BOC: tert-Butoxycarbonyl
DME: 1,2-Dimethoxyethane
DMF: Dimethylformamide
TBAF: Tetrabutylammonium fluoride
EDC: N-Ethyl-N′-(3-dimethyaminopropyl)-carbodiimide hydrochloride
THP: Tetrahydropyranyl
TROC: Trichloroethoxycarbonyl
TPTU: O-(1,2-Dihydro-2-oxo-1-pyridyl)-N,N,N′N′-tetramethyluronium-tetrafluoroborate
HBTU: O-(1H-Benzotriazol-1-yl)-N,N,N′N′-tetramethyluronium-tetrafluorophosphate
SEM: 2-(Trimethylsilyl)-ethoxymethyl

### Example 1

(a) A solution of 23.6 g (100 mmol) of 1,3-dibromobenzene in 250 ml of absolute ether was cooled to −75° C. A solution of 62.5 ml (100 mmol) of n-butyllithium (1.6 M in hexane) was added dropwise within 45 minutes. The resulting suspension was stirred at −75° C. for 2.5 hours. Subsequently, a solution of 19.0 g (100 mmol) of 1-benzyl-4-piperidone in 100 ml of absolute ether was added dropwise within 30 minutes at −70° C. to −75° C. and thereafter the mixture was stirred for 2 hours. Subsequently, the mixture was partitioned between ether and saturated ammonium chloride solution, the organic phase was dried over magnesium sulfate and finally the solvent was removed under reduced pressure. The crude product was purified by chromatography on silica gel with a 1:1 mixture of methylene chloride and hexane as the eluent. There were obtained 28.3 g (82% of theory) of 1-benzyl-4-(3-bromophenyl)-piperidin-4-ol as a yellow oil; MS: 345, 347 (M)⁺.

(b) A solution of 28.2 g (81.4 mmol) of 4-(3-bromophenyl)-piperidin-4-ol in 600 ml of toluene was treated with 30 g (157 mmol) of p-toluenesulfonic acid monohydrate and heated to reflux on a water separator for 4 hours. Subsequently, the reaction mixture was cooled to room temperature and adjusted to pH 10 with 3 N sodium hydroxide solution. Thereafter, it was firstly extracted three times with 500 ml of methylene chloride. The combined organic phases were washed three times with 200 ml of water each time, dried over magnesium sulfate and then evaporated under reduced pressure. The crude product was purified by chromatography on silica gel with a 1:1 mixture of methylene chloride and hexane as the eluent. There were obtained 9.5 g (36% of theory) of 1-benzyl-4-(3-bromophenyl)-1,2,3,6-tetrahydro-pyridine as a light yellow oil; MS: 327, 329 (M+H)⁺.

(c) 3.15 g (83.3 mmol) of sodium borohydride were added portionwise at room temperature to a suspension of 9.5 g (28.9 mmol) of 1-benzyl-4-(3-bromophenyl)-1,2,3,6-tetrahydro-pyridine in 65 ml of absolute dimethoxyethane (DME). Thereafter, a solution of 17.7 ml (20.0 g 140.9 mmol) of boron trifluoride etherate in 11 ml of DME was added dropwise at 15-20° C. and the mixture was stirred at room temperature for 2 hours. Subsequently, a solution of 18.3 g (326 mmol) of potassium hydroxide in 100 ml of water was added dropwise within 30 minutes at 20-25° C. Finally, 55 ml of 30% hydrogen peroxide solution were added dropwise within 30 minutes at 20-25° C. The mixture was stirred at room temperature for 30 minutes and heated to reflux for 3 hours. After cooling the reaction mixture the separated boric acid was filtered off. Subsequently, the filtrate was partitioned between methylene chloride and water, the organic phase was dried over magnesium sulfate and the solvent was removed under reduced pressure. The crude product was purified by chromatography on silica gel with a 1:1 mixture of ethyl acetate and methylene chloride as the eluent. There were obtained 6.3 g (63% of theory) of (3RS,4RS)-1-benzyl-4-(3-bromophenyl)-piperidin-3-ol as a colorless oil. MS: 345, 347 (M)⁺.

(d) A solution of 691 mg (2.00 mmol) of (3RS,4RS)-1-benzyl-4-(3-bromophenyl)-piperidin-3-ol in 3 ml of absolute tetrahydrofuran was treated with 163 mg (2.20 mmol) of lithium carbonate and cooled to −50° C. A solution of 722 mg (4.00 mmol) of β-trimethylsilylethyl chloroformate [Synthesis 346 (1987)] in 4 ml of toluene was added dropwise thereto at −50° C. The reaction mixture was warmed to room temperature and stirred for 24 hours. Subsequently, the mixture was partitioned between methylene chloride and water, the organic phase was dried over magnesium sulfate and finally the solvent was removed under reduced pressure. The crude product (0.8 g) was purified by chromatography on silica gel with methylene chloride as the eluent. There were obtained 470 mg (43% of theory) of 2-trimethylsilylethyl (3RS,4RS)-4-(3-bromophenyl)-3-(2-trimethylsilylethoxycarbonyloxy)-piperidine-1-carboxylate as a light yellow oil, which was used directly in the next step.

(e) A solution of 470 mg (0.863 mmol) of 2-trimethylsilylethyl (3RS,4RS)-4-(3-bromophenyl)-3-(2-trimethylsilyl-ethoxy-carbonyloxy)-piperidine-1-carboxylate in 3 ml of absolute tetrahydrofuran was treated with 2.65 ml (2.91 mmol) of tetrabutylammonium fluoride solution (1.1 M in THF) and stirred at room temperature for 2.5 hours. Subsequently, the mixture was partitioned between methylene chloride and saturated sodium carbonate solution, the organic phase was dried over magnesium sulfate and finally the solvent was removed under reduced pressure. The crude product (440 mg) was purified by chromatography on silica gel with a 6.5:1:0.1 mixture of methylene chloride, MeOH and 25% ammonia as the eluent. There were obtained 180 mg (81% of theory) of (3RS,4RS)-4-(3-bromophenyl)-piperidin-3-ol as a light yellow oil. MS: 255, 257 (M)⁺.

(f) A solution of 180 mg (0.702 mmol) of (3RS,4RS)-4-(3-bromophenyl)-piperidin-3-ol in 1 ml of absolute dimethylformamide was treated at 0° C. with 0.1 ml (73 mg, 0.72 mmol) of triethylamine. A solution of 167 mg (76.5 mmol) of di-tert-butyl dicarbonate in 0.5 ml of dimethylformamide was added thereto at 0° C. The mixture was warmed to room temperature and stirred for 20 hours. The solvent was distilled off at 50-55° C. at 0.1 mm Hg. Subsequently, the residue obtained was partitioned between methylene chloride and water, the organic phase was dried over magnesium sulfate and finally the solvent was removed under reduced pressure. The crude product was purified by chromatography on silica gel with a 4:1 mixture of methylene chloride and ethyl acetate. There were obtained 220 mg (92% of theory) of tert-butyl (3RS,4RS)-4-(3-bromophenyl)-3-hydroxy-piperidine-1-carboxylate as a colorless solid; MS: 299, 301 (M-C₄H₈)⁺.

(g) A solution of 168 mg (0.47 mmol) of tert-butyl (3RS,4RS)-4-(3-bromophenyl)-3-hydroxy-piperidine-1-carboxylate and 157 mg (0.71 mmol) of 2-bromomethylnaphthalene in 2 ml of dimethylformamide was treated with 28 mg (0.7 mmol) of sodium hydride (60% dispersion in refined oil) and the mixture was stirred at room temperature for 3 hours. Subsequently, the reaction mixture was partitioned between ethyl acetate and water, the organic phase was dried over magnesium sulfate and finally the solvent was removed under reduced pressure. The crude product was purified by chromatography on silica gel with a 1:4 mixture of ethyl acetate and hexane as the eluent. There were obtained 173 mg (74% of theory) of tert-butyl (3RS,4RS)-4-(3-bromophenyl)-3-(naphthalen-2-ylmethoxy)-piperidine-1-carboxylate as a light yellow oil; MS: 439, 441 (M-C₄H₈)⁺.

(h) A solution of 173 mg (0.35 mmol) of tert-butyl (3RS,4RS)-4-(3-bromophenyl)-3-(naphthalen-2-ylmethoxy)-piperidine-1-carboxylate in 6 ml of methanol was treated with 6 ml of a 2 N solution of hydrogen chloride in MeOH and stirred at 50° C. for 4 hours. Subsequently, the mixture was partitioned between ethyl acetate and a 5% sodium hydrogen carbonate solution, the organic phase was dried over magnesium sulfate and finally the solvent was removed under reduced pressure. The crude product was purified by chromatography on silica gel with a 10:1:0.1 mixture of methylene chloride, MeOH and 25% ammonia and the eluent. There were obtained 126 mg (91% of theory) of (3RS,4RS)-4-(3-bromophenyl)-3-(naphthalen-2-ylmethoxy)-piperidine as a light yellow oil. MS: 396, 398 (M+H)⁺.

### Example 2

The following compounds were obtained by cleavage of the BOC group in an analogous manner to that described in Example 1 (h):

1)—(3RS,4RS)-3-(4-Methoxy-benzyloxy)-4-phenyl-piperidine as a light yellow oil, MS: 298 (M+H)⁺, from tert-butyl (3RS,4RS)-3-(4-methoxy-benzyloxy)-4-phenyl-piperidine-1-carboxylate;

2)—(3RS,4RS)-4-(4-bromophenyl-3-(4-methoxy-benzyloxy)-piperidine as a colorless oil, MS: 376, 378 (M+H)⁺, from tert-butyl (3RS,4RS)-4-(4-bromophenyl-3-(4-methoxy-benzyloxy)-piperidine-1-carboxylate;

3)—(3RS,4RS)-3-(4-methoxy-benzyloxy)-4-(3-trifluoromethylphenyl)-piperidine as a colorless oil, MS: 366 (M+H)⁺, from tert-butyl (3RS,4RS)-3-(4-methoxy-benzyloxy)-4-(3-trifluoromethylphenyl)-piperidine-1-carboxylate;

4)—(3RS,4RS)-3-(4-methoxy-benzyloxy)-4-p-tolyl-piperidine as a colorless solid, MS: 312 (M+H)⁺, from tert-butyl (3RS,4RS)-3-(4-methoxy-benzyloxy)-4-p-tolyl-piperidine-1-carboxylate;

5)—(3RS,4RS)-3-(naphthalen-2-ylmethoxy)-4-phenyl-piperidine as a light yellow oil, MS: 318 (M+H)⁺, from tert-butyl (3RS,4RS)-3-(naphthalen-2-ylmethoxy)-4-phenyl-piperidine-1-carboxylate;

6)—(3RS,4RS)-4-(4-bromophenyl)-3-(naphthalen-2-ylmethoxy)-piperidine as a colorless solid, MS: 396, 398 (M+H)⁺, from tert-butyl (3RS,4RS)-4-(4-bromophenyl)-3-(naphthalen-2-ylmethoxy)-piperidine-1-carboxylate;

7)—(3RS,4RS)-3-(naphthalen-2-ylmethoxy)-4-(3-trifluoromethyl-phenyl)-piperidine as a colorless oil, MS: 386 (M+H)⁺, from tert-butyl (3RS,4RS)-3-(naphthalen-2-ylmethoxy)-4-(3-trifluoromethyl-phenyl)-piperidine-1-carboxylate;

8)—(3RS,4RS)-4-cyclohexyl-3-(naphthalen-2-ylmethoxy)-piperidine as a light yellow oil, MS: 324 (M+H)⁺, from tert-butyl (3RS,4RS)-4-cyclohexyl-3-(naphthalen-2-ylmethoxy)-piperidine-1-carboxylate

9)—(3RS,4RS)-3-(naphthalen-2-ylmethoxy)-4-p-tolyl-piperidine as a colorless solid, MS: 332 (M+H)⁺, from tert-butyl (3RS,4RS)-3-(naphthalen-2-ylmethoxy)-4-p-tolyl-piperidine-1-carboxylate;

10)—(3RS,4RS)-4-naphthalen-2-yl-3-(naphthalen-2-ylmethoxy)-piperidine as a colorless oil, MS: 367 (M)⁺, from tert-butyl (3RS,4RS)-4-naphthalen-2-yl-3-(naphthalen-2-ylmethoxy)-piperidine-1-carboxylate;

11)—(3RS,4RS)-3-(naphthalen-2-ylmethoxy)-4-(5,6,7,8-tetrahydro-naphthalen-2-yl)-piperidine as a colorless oil, MS: 372 (M+H)⁺, from tert-butyl (3RS,4RS)-3-(naphthalen-2-ylmethoxy)-4-(5,6,7,8-tetrahydro-naphthalen-2-yl)-piperidine-1-carboxylate;

12)—(3RS,4RS)-4-naphthalen-1-yl-3-(naphthalen-2-ylmethoxy)-piperidine as a colorless solid, MS: 367 (M)⁺, from tert-butyl (3RS,4RS)-4-naphthalen-1-yl-3-(naphthalen-2-ylmethoxy)-piperidine-1-carboxylate;

13)—(3RS,4RS)-4-(3,4-dimethoxy-phenyl)-3-(naphthalen-2-ylmethoxy)-piperidine as a colorless resin, MS: 377 (M)⁺, from tert-butyl (3RS,4RS)-4-(3,4-dimethoxy-phenyl)-3-(naphthalen-2-ylmethoxy)-piperidine-1-carboxylate;

14)—(3RS,4RS)-4-acenaphthen-5-yl-3-(naphthalen-2-ylmethoxy)-piperidine as a colorless solid, MS: 394 (M+H)⁺, from tert-butyl (3RS,4RS)-4-acenaphthen-5-yl-3-(naphthalen-2-ylmethoxy)-piperidine-1-carboxylate;

15)—(3RS,4RS)-4-(4-chlorophenyl)-3-(3-phenoxy-benzyloxy)-piperidine as a colorless solid, MS: 394, 396 (M+H)⁺, from tert-butyl (3RS,4RS)-4-(4-chlorophenyl)-3-(3-phenoxy-benzyloxy)-piperidine-1-carboxylate;

16)—(3RS,4SR)-3-(naphthalen-2-ylmethoxy)-4-phenyl-piperidine hydrochloride as a colorless powder, MS: 318 (M+H)⁺, from tert-butyl (3RS,4SR)-3-(naphthalen-2-ylmethoxy)-4-phenyl-piperidine-1-carboxylate.

The BOC compounds used as the starting materials were prepared as follows:

The following compounds were obtained in an analogous manner to that described in Example 1 (b)-(c) and (f)-(g):

(a) From 4-phenyl-piperidin-4-ol there was obtained by elimination 4-phenyl-1,2,3,6-tetrahydro-pyridine as a light yellow oil; MS: 159 (M)⁺. Subsequent hydroboration gave (3RS,4RS)-4-phenyl-piperidin-3-ol as a colorless solid; MS: 177 (M)⁺. Introduction of the BOC group yielded tert-butyl (3RS,4RS)-3-hydroxy-4-phenyl-piperidine-1-carboxylate as a colorless solid; MS: 277 (M)⁺. After alkylation with 4-methoxybenzyl bromide there was obtained tert-butyl (3RS,4RS)-3-(4-methoxy-benzyloxy)-4-phenyl-piperidine-1-carboxylate as a colorless solid; MS: 340 (M-C₄H₉)⁺.

(b) From 4-(4-bromophenyl)-piperidin-4-ol there was obtained by elimination 4-(4-bromophenyl)-1,2,3,6-tetrahydro-pyridine as a light yellow solid; MS: 237, 239 (M)⁺. Subsequent hydroboration gave (3RS,4RS)-4-(4-bromophenyl)-piperidin-3-ol as a colorless solid; MS: 255, 257 (M)⁺. Introduction of the BOC group yielded tert-butyl (3RS,4RS)-4-(4-bromophenyl)-3-hydroxy-piperidine-1-carboxylate as a colorless solid; MS: 299, 301 (M-C₄H.sub.8)⁺. After alkylation with 4-methoxybenzyl bromide there was obtained tert-butyl (3RS,4RS)-4-(4-bromophenyl)-3-(4-methoxy-benzyloxy)-piperidine-1-carboxylate as a light yellow oil; MS: 418, 420 (M-C₄H₉)⁺.

(c) From 4-(3-trifluoromethylphenyl)-piperidin-4-ol there was obtained by elimination 4-(3-trifluoromethylphenyl)-1,2,3,6-tetrahydro-pyridine as a colorless solid; MS: 227 (M)⁺. Subsequent hydroboration gave (3RS,4RS)-4-(3-trifluoromethylphenyl)-piperidin-3-ol as a colorless solid; MS: 245 (M)⁺. Introduction of the BOC group yielded tert-butyl (3RS,4RS)-3-hydroxy-4-(3-trifluoromethylphenyl)-piperidine-1-carboxylate as a colorless solid; MS: 289 (M-C₄ H.sub.8)⁺. After alkylation with 4-methoxybenzyl bromide there was obtained tert-butyl (3RS,4RS)-3-(4-methoxybenzyloxy)-4-(3-trifluoromethylphenyl)-piperidine-1-carboxylate as a light yellow oil; MS: 408 (M-C₄H₉)⁺.

(d) From 1-benzyl-4-(p-tolyl)-piperidin-4-ol there was obtained by elimination 1-benzyl-4-(p-tolyl)-1,2,3,6-tetrahydro-pyridine as a light yellow solid; MS: 263 (M)⁺. Subsequent hydroboration gave (3RS,4RS)-1-benzyl-4-(p-tolyl)-piperidin-3-ol as a colorless solid; MS: 281 (M)⁺.

(e) A solution of 2.5 g (8.9 mmol) of (3RS,4RS)-1-benzyl-4-(p-tolyl)-piperidin-3-ol in 100 ml of methanol was hydrogenated at 5 bar at room temperature for 18 hours using a palladium (10%)-carbon catalyst. For the working up, the catalyst was filtered off, washed with methanol and the solution obtained was evaporated under reduced pressure. For purification, the residue was chromatographed on silica gel using a 5:1:0.1 mixture of methylene chloride, methanol and ammonia as the eluent. There were obtained 1.15 g (68% of theory) of (3RS,4RS)-4-(p-tolyl)-piperidin-3-ol as a colorless solid; MS: 191 (M)⁺.

(f) From (3RS,4RS)-4-(p-tolyl)-piperidin-3-ol by introduction of the BOC group there was obtained tert-butyl (3RS,4RS)-3-hydroxy-4-(p-tolyl)-piperidine-1-carboxylate as a colorless solid; MS: 291 (M)⁺. After alkylation [with] 4-methoxybenzyl bromide there was obtained tert-butyl (3RS,4RS)-3-(4-methoxy-benzyloxy)-4-(p-tolyl)-piperidine-1-carboxylate as a colorless oil; MS: 354 (M-C₄H₉)⁺.

The following compounds were obtained in an analogous manner to that described in Example 1 (g):

(g) By alkylating tert-butyl (3RS,4RS)-3-hydroxy-4-phenyl-piperidine-1-carboxylate with 2-bromomethylnaphthalene there was obtained tert-butyl (3RS,4RS)-3-(napththalen-2-ylmethoxy)-4-phenyl-piperidine-1-carboxylate as a light yellow oil; MS: 417 (M)⁺.

(h) By alkylating tert-butyl (3RS,4RS)-4-(4-bromophenyl)-3-hydroxy-piperidine-1-carboxylate with 2-bromomethyl-naphthalene there was obtained tert-butyl (3RS,4RS)-4-(4-bromophenyl)-3-(naphthalen-2-ylmethoxy)-piperidine-1-carboxylate as a colorless solid; MS: 495, 497 (M)⁺.

(i) By alkylating tert-butyl (3RS,4RS)-3-hydroxy-4-(3-trifluoromethylphenyl)-piperidine-1-carboxylate with 2-bromomethylnaphthalene there was obtained tert-butyl (3RS,4RS)-3-(naphthalen-2-ylmethoxy)-4-(3-trifluoromethyl-phenyl)-piperidine-1-carboxylate as a light yellow oil; MS: 485 (M)⁺.

(j) A solution of 4.0 g tert-butyl (13.8 mmol) of tert-butyl (3RS,4RS)-3-hydroxy-4-phenyl-piperidine-1-carboxylate in 100 ml of methanol was hydrogenated at 150 bar at 100° C. for 18 hours using a rhodium(5%)-aluminium oxide catalyst. For the working up, the catalyst was filtered off, washed with methanol and the solution obtained was evaporated under reduced pressure. For purification, the residue was chromatographed on silica gel using a 4:1 mixture of hexane and ethyl acetate as the eluent. There were obtained 2.32 g (59% of theory) of tert-butyl (3RS,4RS)-4-cyclohexyl-3-(naphthalen-2-ylmethoxy)-piperidine-1-carboxylate as a colorless solid; MS: 283 (M)⁺.

(k) By alkylating tert-butyl (3RS,4RS)-4-cyclohexyl-3-(naphthalen-2-ylmethoxy)-piperidine-1-carboxylate with 2-bromomethylnaphthalene there was obtained tert-butyl (3RS,4RS)-4-cyclohexyl-3-(naphthalen-2-ylmethoxy)-piperidine-1-carboxylate as a light yellow oil; MS: 423 (M)⁺.

(l) By alkylating tert-butyl (3RS,4RS)-3-hydroxy-4-(p-tolyl)-piperidine-1-carboxylate with 2-bromomethylnaphthalene there was obtained tert-butyl (3RS,4RS)-3-(naphthalen-2-ylmethoxy)-4-p-tolyl-piperidine-1-carboxylate as a colorless oil; MS: 431 (M)⁺.

The remaining starting materials were obtained as follows:

(m) From 2-bromonaphthalene and 1-benzyl-4-piperidone there was obtained in an analogous manner to Example 1(a) 1-benzyl-4-naphthalen-2-yl-piperidin-4-ol as a light yellow oil; MS: 317 (M)⁺. Elimination in an analogous manner to that described in Example 1(b) yielded 1-benzyl-4-naphthalen-2-yl-1,2,3,6-tetrahydro-pyridine as a light brown oil; MS: 299 (M)⁺. Subsequent cleavage of the benzyl group analogously to Example 1(d) gave 2-trimethylsilylethyl 4-naphthalen-2-yl-1,2,3,6-tetrahydro-pyridine-1-carboxylate as a colorless solid; 4-MS: 325 (M-C₂H₄)⁺. By treatment with tetrabutylammonium fluoride in tetrahydrofuran analogously to Example 1(e) there was obtained 4-naphthalen-2-yl-1,2,3,6-tetrahydro-pyridine as a colorless solid; MS: 209 (M)⁺. Subsequent hydroboration in an analogous manner to that described in Example 1(c) gave (3RS,4RS)-naphthalen-2-yl-4-piperidin-3-ol as a colorless solid; MS: 227 (M)⁺. Introduction of the BOC group in analogy to Example 1(f) yielded tert-butyl (3RS,4RS)-3-hydroxy-4-naphthalen-2-yl-piperidine-1-carboxylate as a colorless oil; MS: 327 (M)⁺. After alkylation with 2-bromomethylnaphthalene in an analogous manner to the procedure described in Example 1(g) there was obtained tert-butyl (3RS,4RS)-4-naphthalen-2-yl-3-(naphthalen-2-ylmethoxy)-piperidine-1-carboxylate as a colorless oil; MS: 467 (M)⁺.

(n) In an analogous manner to that described in Example 2(e), by catalytically hydrogenating (3RS,4RS)-1-benzyl-4-naphthalen-2-yl-piperidin-3-ol there was obtained (3RS,4RS)-4-(5,6,7,8-tetrahydro-naphthalen-2-yl)-piperidin-3-ol as a colorless solid; MS: 231 (M)⁺. Introduction of the BOC group in an analogous manner to that described in Example 1(f) yielded tert-butyl (3RS,4RS)-3-hydroxy-4-(5,6,7,8-tetrahydro-naphthalen-2-yl)-piperidine 1-carboxylate as a colorless oil; MS: 331 (M)⁺. After alkylation with 2-bromomethylnaphthalene in an analogous manner to the procedure described in Example 1(g) there was obtained tert-butyl (3RS,4RS)-3-(naphthalen-2-ylmethoxy)-4-(5,6,7,8-tetrahydro-naphthalen-2-yl)-piperidine-1-carboxylate as a colorless oil; MS: 471 (M)⁺.

(o) From 1-benzyl-4-naphthalen-1-yl-1,2,3,6-tetrahydro-pyridine (EP-A-372 776) by hydroboration in an analogous manner to Example 1(c) there was obtained (3RS,4RS)-1-benzyl-4-naphthalen-1-yl-piperidin-3-ol as a colorless solid; MS: 317 (M)⁺. The benzyl group was removed by catalytic hydrogenation [palladium (10%)-charcoal, ethanol, 80° C., 24 hours, 50 bar, 21% of theory] in an analogous manner to that described in Example 2(e). (3RS,4RS)-4-Naphthalen-1-yl-piperidin-3-ol was obtained as a beige solid; MS: 227 (M)⁺. Introduction of the BOC group in an analogous manner to that described in Example 1(f) yielded tert-butyl (3RS,4RS)-3-hydroxy-4-naphthalen-1-yl-piperidine-1-carboxylate as a colorless solid; MS: 327 (M)⁺. After alkylation with 2-bromomethylnaphthalene, in analogy to the procedure described in Example 1(g) there was obtained tert-butyl (3RS,4RS)-4-naphthalen-1-yl-3-(naphthalen-2-ylmethoxy)-piperidine-1-carboxylate as a colorless solid; MS: 467 (M)⁺.

(p) From 1-benzyl-4-(3,4-dimethoxy-phenyl)-1,2,3,6-tetrahydro-pyridine (JP 60 146 872) by hydroboration in an analogous manner to that described in Example 1(c) there was obtained (3RS,4RS)-1-benzyl-(3,4-dimethoxy-phenyl)-piperidin-3-ol as a colorless solid; MS: 327 (M)⁺. The benzyl group was removed by catalytic hydrogenation [palladium (10%)-charcoal, methanol, room temperature, 18 hours, 5 bar, 81% of theory] in an analogous manner to that described in Example 2(e). (3RS,4RS)-4-(3,4-Dimethoxy-phenyl)-piperidin-3-ol was obtained as a colorless solid; MS: 237 (M)⁺. Introduction of the BOC group in an analogous manner to that described in Example 1(f) yielded tert-butyl (3RS,4RS)-3-hydroxy-4-(3,4-dimethoxy-phenyl)-piperidine-1-carboxylate as a light yellow oil; MS: 337 (M)⁺. After alkylation with 2-bromomethylnaphthalene in analogy to the procedure described in Example 1(g) there was obtained tert-butyl (3RS,4RS)-4-(3,4-dimethoxy-phenyl)-3-(naphthalen-2-ylmethoxy)-piperidine-1-carboxylate as a light yellow oil; MS: 477 (M)⁺.

(q) From 5-bromo-acenaphthene and 1-benzyl-4-piperidone there was obtained in an analogous manner to Example 1(a) 4-acenaphthen-5-yl-1-benzyl-piperidin-4-ol as a yellow oil; MS: 343 (M)⁺. Elimination in an analogous manner to that described in Example 1(b) yielded 4-acenaphthen-5-yl-1-benzyl-1,2,3,6-tetrahydro-pyridine as a light brown oil; MS: 325 (M)⁺. Subsequent hydroboration in an analogous manner to that described in Example 1(c) gave (3RS,4RS)-1-benzyl-acenaphthen-5-yl-4-piperidin-3-ol as a yellow oil; MS: 343 (M)⁺. The benzyl group was removed by catalytic hydrogenation [palladium (10%)-charcoal, methanol, room temperature, 18 hours, 5 bar, 95% of theory] in an analogous manner to that described in Example 2(e). (3RS,4RS)-4-Acenaphthen-5-yl-piperidin-3-ol was obtained as a colorless solid; MS: 253 (M)⁺. Introduction of the BOC group in an analogous manner to that described in Example 1(f) yielded tert-butyl (3RS,4RS)-4-acenaphthen-5-yl-3-hydroxy-piperidine-1-carboxylate as a colorless solid; MS: 353 (M)⁺. After alkylation with 2-bromomethylnaphthalene in analogy to the procedure described in Example 1(g) there was obtained tert-butyl (3RS,4RS)-4-acenaphthen-5-yl-3-(naphthalen-2-ylmethoxy)-piperidine-1-carboxylate as a yellow solid; MS: 493 (M)⁺.

(r) From 4-(4-chlorophenyl)-piperidin-4-ol by elimination in an analogous manner to that described in Example 1(b) there was obtained 4-(4-chlorophenyl)-1,2,3,6-tetrahydro-pyridine as a light yellow solid; MS: 193, 195 (M)⁺. Hydroboration in an analogous manner as Example 1(c) gave (3RS,4RS)-4-(4-chlorophenyl)-piperidin-3-ol as a colorless solid; MS: 211, 213 (M)⁺. Introduction of the BOC group in an analogous manner to that described in Example 1(f) yielded tert-butyl (3RS,4RS)-4-(4-chlorophenyl)-3-hydroxy-piperidine-1-carboxylate as a colorless solid; MS: 255, 257 (M-C₄H.sub.8)⁺. After alkylation with 4-phenoxybenzyl chloride in analogy to the procedure described in Example 1(g) there was obtained tert-butyl (3RS,4RS)-4-(4-chlorophenyl)-3-(4-phenoxy-benzyloxy)-piperidine-1-carboxylate as a colorless oil; MS: 437, 439 (M-C₄H₉)⁺.

(s) From (3RS,4SR)-4-phenyl-piperidin-3-ol [J. A. Gauthier et al., U.S. Pat. No. 4,132,710] by introduction of the BOC group there was obtained tert-butyl (3RS,4SR)-3-hydroxy-4-phenyl-piperidine-1-carboxylate as a colorless solid; m.p.: 134-134.5° C. Subsequent alkylation with 2-bromomethylnaphthalene gave tert-butyl (3RS,4SR)-3-(naphthalen-2-ylmethoxy)-4-phenyl-piperidine-1-carboxylate as a colorless solid; MS: 417 (M)⁺.

### Example 3

130 mg (0.31 mmol) of tert-butyl (3RS,4RS)-4-(4-fluorophenyl)-3-(4-methoxy-benzyloxy)-piperidine-1-carboxylate were dissolved in 5 ml of methanol, treated with 5 ml of a 2 N solution of hydrogen chloride in methanol and stirred at 50° C. for 4 hours. Subsequently, the mixture was partitioned between ethyl acetate and aqueous 5% sodium hydrogen carbonate solution, the organic phase was dried over magnesium sulfate and finally the solvent was distilled off under reduced pressure. For purification, the crude product was chromatographed on silica gel with a 10:1:0.1 mixture of methylene chloride, methanol and ammonia as the eluent. There were obtained 76 mg (78% of theory) of (3RS,4RS)-4-(4-fluorophenyl)-3-(4-methoxy-benzyloxy)-piperidine as a colorless oil. MS: 316 (M+H)⁺.

The tert-butyl (3RS,4RS)-4-(4-fluorophenyl)-3-(4-methoxy-benzyloxy)-piperidine-1-carboxylate used as the starting material was prepared as follows:

(a) 20.0 g (93.6 mmol) of (3RS,4RS)-4-(4-fluorophenyl)-1,2,3,6-tetrahydropyridine hydrochloride were suspended in 160 ml of absolute dimethoxyethane. 10.6 g (280 mmol) of sodium borohydride were added portionwise at room temperature. Thereafter, a solution of 62 ml (500 mmol) of boron trifluoride etherate in 30 ml of dimethoxyethane was added dropwise at 15-20° C. and the mixture was stirred at room temperature for 2.5 hours. Subsequently, a solution of 65 g (1.16 mmol) of potassium hydroxide in 340 ml of water was added dropwise at 20-25° C. within 60 minutes. 55 ml of hydrogen peroxide solution (30%) were then added dropwise at 20-25° C. within 30 minutes. The mixture was stirred at room temperature for 30 minutes and boiled under reflux for 3 hours. After cooling the reaction mixture the precipitated boric acid was filtered off. Subsequently, the filtrate was partitioned between methylene chloride and water, the organic phase was dried over magnesium sulfate and the solvent was distilled off under reduced pressure. The crude product was purified by chromatography on silica gel with a 3:1:0.1 mixture of methylene chloride, methanol and ammonia as the eluent. There were obtained 9.1 g (50% of theory) of (3RS,4RS)-4-(4-fluorophenyl)-piperidin-3-ol as a colorless oil. MS: 195 (M)⁺.

(b) 4.10 g (21.0 mmol) of (3RS,4RS)-4-(4-fluorophenyl)-piperidin-3-ol were dissolved in 35 ml of absolute dimethylformamide. Thereto there were added at 0° C. 3.2 ml (23.0 mmol) of triethylamine and subsequently dropwise a solution of 5.04 g (23.1 mmol) of di-tert-butyl dicarbonate in 15 ml of dimethylformamide. The mixture was warmed to room temperature and stirred for 20 hours. The solvent was distilled off at 0.1 mm Hg at 50-55° C. Subsequently, the residue obtained was partitioned between methylene chloride and water, the organic phase was dried over magnesium sulfate and finally the solvent was removed under reduced pressure. The crude product (7.09 g) was purified by chromatography on silica gel with a 2:3 mixture of ethyl acetate and hexane as the eluent. There were obtained 5.45 mg (88% of theory) of tert-butyl (3RS,4RS)-4-(4-fluorophenyl)-3-hydroxy-piperidine-1-carboxylate; MS: 239 (M-C₄H₈)⁺.

(c) 200 mg (0.68 mmol) of tert-butyl (3RS,4RS)-4-(4-fluorophenyl)-3-hydroxy-piperidine-1-carboxylate and 159 mg (1.01 mmol) of 4-methoxybenzyl chloride were dissolved in 3 ml of dimethylformamide. 40 mg (1.01 mmol) of a 60% sodium hydride suspension were added and the mixture was stirred at room temperature for 3 hours. Subsequently, the reaction mixture was partitioned between ethyl acetate and water, the organic phase was dried over magnesium sulfate and finally the solvent was removed under reduced pressure. The crude product was purified by chromatography on silica gel with a 1:3 mixture of ethyl acetate and hexane as the eluent. There were obtained 250 mg (90% of theory) of tert-butyl (3RS,4RS)-4-(4-fluorophenyl)-3-(4-methoxy-benzyloxy)-piperidine-1-carboxylate as a light yellow oil; MS: 358 (M-C₄H₉)⁺.

### Example 4

The following compounds were prepared in an analogous manner to that described in Example 3:

1) (3RS,4RS)-4-(4-fluorophenyl)-3-(naphthalen-2-ylmethoxy)-piperidine as a colorless oil, MS: 336 (M+1)⁺, from tert-butyl (3RS,4RS)-4-(4-fluorophenyl)-3-(naphthalen-2-yl-methoxy)-piperidine-1-carboxylate;

2) (3RS,4RS)-3-(3-benzyloxy-benzyloxy)-4-(4-fluorophenyl)-piperidine as a colorless solid, MS: 392 (M+1)⁺, from tert-butyl (3RS,4RS)-3-(3-benzyloxy-benzyloxy)-4-(4-fluorophenyl)-piperidine-1-carboxylate;

3) (3RS,4RS)-4-(4-fluorophenyl)-3-(4-methoxy-quinazolin-2-ylmethoxy)-piperidine as a colorless solid, MS: 368 (M+1)⁺, from tert-butyl (3RS,4RS)-4-(4-fluorophenyl)-3-(4-methoxy-quinazolin-2-ylmethoxy)-piperidine-1-carboxylate;

4) (3RS,4RS)-3-(benzo[b]thiophen-5-ylmethoxy)-4-(4-fluorophenyl)-piperidine as a colorless solid, MS: 342 (M+1)⁺, from tert-butyl (3RS,4RS)-3-(benzo[b]thiophen-5-ylmethoxy)-4-(4-fluorophenyl)-piperidine-1-carboxylate;

5) (3RS,4RS)-4-(4-fluorophenyl)-3-(indan-5-ylmethoxy)-piperidine as a colorless solid, MS: 326 (M+1)⁺, from tert-butyl (3RS,4RS)-4-(4-fluorophenyl)-3-(indan-5-yl-methoxy)-piperidine-1-carboxylate,

6) (3RS,4RS)-4-(4-fluoro-phenyl)-3-(5,6,7,8-tetrahydro-naphthalen-2-ylmethoxy)-piperidine as a colorless solid, MS: 340 (M+1)⁺, from tert-butyl (3RS,4RS)-4-(4-fluorophenyl)-3-(5,6,7,8-tetra-hydronaphthalen-2-ylmethoxy)-piperidine-1-carboxylate;

7) (3RS,4RS)-4-(4-fluoro-phenyl)-3-(isoquinolin-6-ylmethoxy)-piperidine as a colorless solid, MS: 337 (M+1)⁺, from tert-butyl (3RS,4RS)-4-(4-fluoro-phenyl)-3-(isoquinolin-6-ylmethoxy)-piperidine-1-carboxylate.

The BOC derivatives used as the starting materials were obtained as follows in an analogous manner to the alkylation procedure described in Example 3 (c):

tert-Butyl (3RS,4RS)-4-(4-fluorophenyl)-3-(naphthalen-2-yl-methoxy)-piperidine-1-carboxylate as a light yellow oil, MS: 436 (M+1)⁺, from tert-butyl (3RS,4RS)-4-(4-fluorophenyl)-3-hydroxy-piperidine-1-carboxylate and 2-naphthylmethyl bromide;

tert-butyl (3RS,4RS)-3-(3-benzyloxy-benzyloxy)-4-(4-fluoro-phenyl)-piperidine-1-carboxylate as a colorless solid, MS: 492 (M+1)⁺, from tert-butyl (3RS,4RS)-4-(4-fluorophenyl)-3-hydroxy-piperidine-1-carboxylate and 3-benzyloxy-benzyl chloride [J. Med. Chem. 31(3), 606 (1988)];

tert-butyl (3RS,4RS)-4-(4-fluorophenyl)-3-(4-methoxy-quinazolin-2-ylmethoxy)-piperidine-1-carboxylate as a colorless solid, MS: 492 (M+1)⁺, from tert-butyl (3RS,4RS)-4-(4-fluoro-phenyl)-3-hydroxy-piperidine-1-carboxylate and 2-bromomethyl-4-methoxy-quinazoline;

tert-butyl (3RS,4RS)-3-(benzo[b]thiophen-5-ylmethoxy)-4-(4-fluoro-phenyl)-piperidine-1-carboxylate as a light yellow resin, MS: 442 (M+1)⁺, from tert-butyl (3RS,4RS)-4-(4-fluorophenyl)-3-hydroxy-piperidine-1-carboxylate and 5-bromomethyl-benzo[b]thiophene [J. Med. Chem. 34(1), 65 (1991)];

tert-butyl (3RS,4RS)-4-(4-fluorophenyl)-3-(indan-5-ylmethoxy)-piperidine-1-carboxylate as a colorless solid, MS: 426 (M+1)⁺, from tert-butyl (3RS,4RS)-4-(4-fluorophenyl)-3-hydroxy-piperidine-1-carboxylate and 5-chloromethylindane [Recl. Trav. Chim. Pays-Bas 77, 792 (1988)];

tert-butyl (3RS,4RS)-4-(4-fluorophenyl)-3-(5,6,7,8-tetrahydro-naphthalen-2-ylmethoxy)-piperidine-1-carboxylate as a light yellow resin, MS: 440 (M+1)⁺, from tert-butyl (3RS,4RS)-4-(4-fluorophenyl)-3-hydroxy-piperidine-1-carboxylate and 6-chloromethyl-1,2,3,4-tetrahydro-naphthalene [J. Chem. Soc. 684 (1941)];

tert-butyl (3RS,4RS)-4-(4-fluoro-phenyl)-3-(isoquinolin-6-ylmethoxy)-piperidine-1-carboxylate as a light yellow resin, MS: 437 (M+1)⁺, from tert-butyl (3RS,4RS)-4-(4-fluorophenyl)-3-hydroxy-piperidine-1-carboxylate and 6-bromomethylisoquinoline hydrobromide.

### 2-Bromomethyl-4-methoxy-quinazoline

(a) By brominating 2-methyl-4-methoxy-quinazoline [Recl. Trav. Chim. Pays-Bas 76, 401 (1957)] with N-bromosuccinimide in carbon tetrachloride in an analogous manner to the procedure described for the preparation of 6-bromomethylquinoxaline [J. Het. Chem. 11, 595 (1974)] from 6-methylquinoxaline there was obtained 2-bromomethyl-4-methoxy-quinazoline as a light yellow solid; MS: 252, 254 (M)⁺.

### 6-Bromomethyl-isoquinoline hydrobromide

(b) From isoquinoline-6-carboxylic acid [J. Am. Chem. Soc. 61, 183 (1939)] by esterification with ethanol/sulfuric acid there was obtained ethyl isoquinoline-6-carboxylate as a colorless solid; MS: 201 (M)⁺. Subsequent reduction yielded 6-isoquinoline-methanol as a yellow solid which was used directly in the next step.

(c) A solution of 190 mg (1.19 mmol) of 6-isoquinoline-methanol in 1 ml of glacial acetic acid was treated with 2 ml of 30% HBr in glacial acetic acid and the mixture was heated at 70° C. for 45 minutes. The reaction mixture was cooled, treated with 20 ml of diethyl ether and stirred at 0° C. for 30 min. The resulting solid was filtered off, washed with diethyl ether and dried in a high vacuum. There was obtained 6-bromomethyl-isoquinoline hydrobromide (73% of theory) as a light brown solid; MS: 221, 223 (M)⁺.

### Example 5

70 mg (0.141 mmol) of β-trimethylsilylethyl (3RS,4RS)-4-(4-fluorophenyl)-3-(4-hydroxy-naphthalen-2-ylmethoxy)-piperidine-1-carboxylate were dissolved in 1.0 ml of tetrabutylammonium fluoride solution (1 M in tetrahydrofuran) and stirred at room temperature for one hour. Subsequently, the mixture was partitioned between methylene chloride and aqueous 5% sodium hydrogen carbonate solution, then the organic phase was dried over magnesium sulfate and finally the solvent was distilled off under reduced pressure. The crude product (72 mg) was purified by chromatography on silica gel with a 10:1:0.1 mixture of methylene chloride, methanol and ammonia as the eluent. There were obtained 41 mg (83% of theory) of (3RS,4RS)-4-(4-fluorophenyl)-3-(4-hydroxy-naphthalen-2-ylmethoxy)-piperidine as a colorless solid. MS: 352 (M+H)⁺.

The β-trimethylsilylethyl (3RS,4RS)-4-(4-fluorophenyl)-3-(4-hydroxy-naphthalen-2-ylmethoxy)-piperidine-1-carboxylate used as the starting material was prepared as follows:

(a) 17.87 g (82.64 mmol) of ethyl 4-hydroxy-naphthalene-2-carboxylate [J. Agric. Chem Soc. Japan 24, 313 (1950)] were suspended in 900 ml of methylene chloride, the suspension was cooled to 0-5° C. and subsequently treated with 17.9 ml (91.02 mmol) of 2-(trimethylsilyl)-ethoxymethyl chloride (SEM chloride) and 28.3 ml (165.31 mmol) of N-ethyldiisopropylamine. The yellow solution was warmed to room temperature and stirred for 2 hours. The solvent was distilled off under reduced pressure and the crude product (58 g), without further purification, was chromatographed on silica gel using a 3:2 mixture of methylene chloride and hexane as the eluent. There were obtained 15.81 g (99% of theory) of ethyl 4-(2-trimethyl-silylethoxy-methoxy)-naphthalene-2-carboxylate as a light yellow oil; MS: 322, 324 (M)⁺.

(b) A solution of 28.31 g (81.70 mmol) of ethyl 4-(2-trimethyl-silylethoxy-methoxy)-naphthalene-2-carboxylate in 480 ml of diethyl ether was added dropwise within 90 minutes at −5 to 0° C. under argon to a suspension of 3.29 g (86.69 mmol) of lithium aluminium hydride in 230 ml of diethyl ether. The reaction mixture was warmed to room temperature and stirred for 2 hours. For the working-up, the mixture was cooled to 0° C., treated dropwise with 25 ml of ethyl acetate and with 50 ml of saturated potassium sodium tartrate solution. A light yellowish solution containing a white precipitate resulted. The solution was warmed to room temperature [and] decanted off from the precipitate. The residue was suspended three times with diethyl ether and the solvent was decanted off each time. The combined organic phases were washed with water, the organic phase was dried over magnesium sulfate and finally the solvent was removed under reduced pressure. The crude product (26.4 g) was chromatographed on silica gel using a 3:7 mixture of ethyl acetate and hexane. There were obtained 23.72 g (95% of theory) of [4-(2-trimethylsilyl-ethoxymethoxy)-naphthalen-2-yl]-methanol as a light yellow oil; MS: 304 (M)⁺.

(c) 23.72 g (77.91 mmol) of [4-(2-trimethylsilyl-ethoxymethoxy)-naphthalen-2-yl]-methanol were dissolved in 350 ml of carbon tetrachloride and the solution was cooled to 0° C. Thereupon, 350 ml of acetonitrile and 26.54 g (1.012 mmol) of triphenylphosphine were added. The light yellow solution was stirred at 0° C. for 30 minutes, warmed to room temperature and stirred for a further 2 hours. A further 10.14 g (38.7 mmol) of triphenylphosphine were added and the reaction mixture was stirred at room temperature for 90 minutes. Subsequently, the mixture was partitioned between methylene chloride and water, the organic phase was dried over magnesium sulfate and finally the solvent was removed under reduced pressure. The crude product was chromatographed on silica gel using a 3:7 mixture of methylene chloride and hexane as the eluent. There were obtained 15.81 g (63% of theory) of 2-chloromethyl-4-(β-trimethylsilylethoxymethoxy)-naphthalene as a light yellow oil; MS: 322, 324 (M)⁺.

(d) A solution of 4.00 g (20.5 mmol) of (3RS,4RS)-4-(4-fluorophenyl)-piperidin-3-ol in 150 ml of ethanol was treated with 2.80 g (26.4 mmol) of sodium carbonate and refluxed. A solution of 2.50 ml (21.1 mmol) of benzyl bromide in 50 ml of ethanol was added dropwise within one hour and thereafter the mixture was held at reflux temperature for 2 hours. The pale brownish suspension was filtered and the filtrate was concentrated under reduced pressure. Subsequently, the residue was partitioned between methylene chloride and water, the organic phase was dried over magnesium sulfate and finally the solvent was distilled off under reduced pressure. The crude product was chromatographed on silica gel using a 2:3 mixture of ethyl acetate and hexane as the eluent. There were obtained 4.34 g (74% of theory) of (3RS,4RS)-1-benzyl-4-(4-fluoro-phenyl)-piperidin-3-ol as a colorless solid; MS: 285 (M)⁺.

(e) In an analogous manner to that described in Example 1(g), by alkylating (3RS,4RS)-1-benzyl-4-(4-fluoro-phenyl)-piperidin-3-ol with 2-chloromethyl-4-(β-trimethylsilylethoxymethoxy)-naphthalene there was obtained (3RS,4RS)-1-benzyl-4-(4-fluoro-phenyl)-3-[4-(2-trimethylsilyl-ethoxymethoxy)-naphthalen-2-ylmethoxy]-piperidine as a light yellow oil; MS: 572 (M+H)⁺.

(f) In an analogous manner to that described in Example 1(d) by cleavage of the benzyl group by means of β-trimethylsilylethyl chloroformate from (3RS,4RS)-1-benzyl-4-(4-fluoro-phenyl)-3-[4-(2-trimethylsilyl-ethoxymethoxy)-naphthalen-2-ylmethoxy]-piperidine there was obtained β-trimethylsilylethyl (3RS,4RS)-4-(4-fluorophenyl)-3-[4-(2-trimethylsilyl-ethoxymethoxy)-naphthalen-2-yl-methoxy]-piperidine-1-carboxylate as a light yellow oil; MS: 626 (M+H)⁺.

(g) 4.65 g (7.43 mmol) of β-trimethylsilylethyl (3RS,4RS)-4-(4-fluorophenyl)-3-[4-(2-trimethylsilyl-ethoxymethoxy)-naphthalen-2-ylmethoxy]-piperidin-1-carboxylate were dissolved in 40 ml of methanol, treated with 40 ml of a 2 N solution of hydrogen chloride in methanol and stirred at 50° C. for 90 minutes. Subsequently, the mixture was partitioned between methylene chloride and aqueous 5% sodium hydrogen carbonate solution, the organic phase was dried over magnesium sulfate and finally the solvent was distilled off under reduced pressure. The crude product (6.8 g) was purified by chromatography on silica gel with a 3:7 mixture of ethyl acetate and hexane. There were obtained 2.93 g (80% of theory) of β-trimethylsilylethyl (3RS,4RS)-4-(4-fluorophenyl)-3-(4-hydroxy-naphthalen-2-ylmethoxy)-piperidine-1-carboxylate as a colorless solid; MS: 496 (M+H)⁺.

### Example 6

The following compounds were obtained in an analogous manner to that described in Example 5:

1)—4-(4-Fluoro-phenyl)-3-(1-hydroxy-naphthalen-2-yl-methoxy)-piperidine as a light brown solid, MS: 351 (M)⁺, from β-trimethylsilylethyl (3RS,4RS)-4-(4-fluorophenyl)-3-(1-hydroxy-naphthalen-2-ylmethoxy)-piperidine-1-carboxylate;

2)—4-(4-fluoro-phenyl)-3-(5-hydroxy-naphthalen-2-yl-methoxy)-piperidine as a colorless solid, MS: 351 (M)⁺, from β-trimethylsilylethyl (−3RS,4RS)-4-(4-fluorophenyl)-3-(5-hydroxy-naphthalen-2-ylmethoxy)-piperidine-1-carboxylate;

3)—4-(4-fluoro-phenyl)-3-(6-hydroxy-naphthalen-2-yl-methoxy)-piperidine as a colorless solid, MS: 351 (M)⁺, from β-trimethylsilylethyl (3RS,4RS)-4-(4-fluorophenyl)-3-(6-hydroxy-naphthalen-2-ylmethoxy)-piperidine-1-carboxylate;

4)—4-(4-fluoro-phenyl)-3-(7-hydroxy-naphthalen-2-yl-methoxy)-piperidine as a light brown solid, MS: 351 (M)⁺, from β-trimethylsilylethyl (3RS,4RS)-4-(4-fluorophenyl)-3-(7-hydroxy-naphthalen-2-ylmethoxy)-piperidine-1-carboxylate;

5)—4-(4-fluoro-phenyl)-3-(8-hydroxy-naphthalen-2-yl-methoxy)-piperidine as a light yellow resin, MS: 352 (M+H)⁺, from β-trimethylsilylethyl (3RS,4RS)-4-(4-fluorophenyl)-3-(8-hydroxy-naphthalen-2-ylmethoxy)-piperidine-1-carboxylate.

The trimethylsilylethyl carbamates used as the starting materials were obtained as follows in analogy to the procedure described in Example 5 (a)-(f):

(a) From methyl 1-hydroxy-naphthalene-2-carboxylate [J. Chem. Soc. 309 (1948)] by introducing the protecting group there was obtained methyl 1-(2-trimethylsilylethoxymethoxy)-naphthalene-2-carboxylate as a light yellow oil, MS: 333 (M+H)⁺.

(b) Reduction of methyl 1-(2-trimethylsilyl-ethoxymethoxy)-naphthalene-2-carboxylate gave [1-(2-trimethyl-silylethoxymethoxy)-naphthalen-2-yl]-methanol as a light yellow oil, MS: 305 (M+H)⁺.

(c) Chlorination of [1-(2-trimethylsilylethoxy-methoxy)-naphthalen-2-yl]-methanol yielded 2-chloro-methyl-1-(β-trimethylsilylethoxymethoxy)-naphthalene as a colorless oil, MS: 322, 324 (M)⁺.

(d) Alkylation of (3RS,4RS)-1-benzyl-4-(4-fluoro-phenyl)-piperidin-3-ol with 2-chloromethyl-1-(β-trimethylsilylethoxy-methoxy)-naphthalene yielded (3RS,4RS)-1-benzyl-3-(4-fluoro-phenyl)-3-[1-(2-trimethylsilylethoxy-methoxy)-naphthalen-2-ylmethoxy]-piperidine as a light yellow oil, MS: 572 (M+H)⁺.

(e) Cleavage of the N-benzyl group from (3RS,4RS)-1-benzyl-3-(4-fluorophenyl)-3-[1-(2-trimethylsilylethoxy-methoxy)-naphthalen-2-ylmethoxy]-piperidine with β-trimethylsilylethyl chloroformate gave β-trimethylsilylethyl (3RS,4RS)-4-(4-fluorophenyl)-3-[1-(2-trimethylsilyl-ethoxymethoxy)-naphthalen-2-ylmethoxy]-piperidine-1-carboxylate as a colorless oil, MS: 626 (M+H)⁺.

(f) Cleavage of the SEM group from β-trimethylsilylethyl (3RS,4RS)-4-(4-fluorophenyl)-3-[1-(2-trimethylsilyl-ethoxymethoxy)-naphthalen-2-ylmethoxy]-piperidine-1-carboxylate yielded β-trimethylsilylethyl (3RS,4RS)-4-(4-fluorophenyl)-3-(1-hydroxy-naphthalen-2-ylmethoxy)-piperidine-1-carboxylate as a colorless oil, MS: 494 (M-H)⁻.

(g) From 5-hydroxy-naphthalene-2-carboxylic acid [Bull. Soc. Chim. Fr., 857 (1953)] there was obtained firstly by esterification with methanol/sulfuric acid methyl 5-hydroxy-naphthalene-2-carboxylate as a light yellow solid, MS: 202 (M)⁺. By introducing the protecting group there was obtained methyl 5-(2-trimethylsilyl-ethoxymethoxy)-naphthalene-2-carboxylate as a light yellow oil, MS: 333 (M+H)⁺.

(h) Reduction of methyl 5-(2-trimethylsilyl-ethoxymethoxy)-naphthalene-2-carboxylate gave [5-(2-trimethylsilyl-ethoxymethoxy)-naphthalen-2-yl]-methanol as a light yellow oil, MS: 305 (M+H)⁺.

(i) Chlorination of [5-(2-trimethylsilyl-athoxymethoxy)-naphthalen-2-yl]-methanol yielded 2-chloro-methyl-5-(β-trimethyl-silylethoxy-methoxy)-naphthalene as a colorless oil, MS: 322, 324 (M)⁺.)

(j) Alkylation of (3RS,4RS)-1-benzyl-4-(4-fluoro-phenyl)-piperidin-3-ol with 2-chloromethyl-5-(β-trimethyl-silylethoxy-methoxy)-naphthalene yielded (3RS,4RS)-1-benzyl-3-(4-fluoro-phenyl)-3-[5-(2-tri-methylsilyl-ethoxymethoxy)-naphthalen-2-ylmethoxy]-piperidine as a light yellow oil, MS: 572 (M+H)⁺.

(k) Cleavage of the N-benzyl group from (3RS,4RS)-1-benzyl-3-(4-fluoro-phenyl)-3-[5-(2-trimethylsilyl-ethoxymethoxy)-naphthalen-2-ylmethoxy]-piperidine with β-trimethylsilylethyl chloroformate gave β-trimethylsilylethyl (3RS,4RS)-4-(4-fluoro-phenyl)-3-[5-(2-trimethylsilyl-ethoxymethoxy)-naphthalen-2-ylmethoxy]-piperidine-1-carboxylate as a colorless oil, MS: 626 (M+H)⁺.

(l) Cleavage of the SEM group in β-trimethylsilylethyl (3RS,4RS)-4-(4-fluorophenyl)-3-[5-(2-trimethylsilyl-ethoxymethoxy)-naphthalen-2-ylmethoxy]-piperidine-1-carboxylate yielded β-trimethylsilylethyl (3RS,4RS)-4-(4-fluorophenyl)-3-(5-hydroxy-naphthalen-2-ylmethoxy)-piperidine-1-carboxylate as a colorless oil, MS: 494 (M-H)⁻.

(m) From ethyl 6-hydroxy-naphthalene-2-carboxylate [J. Chem. Soc. 123, 1654 (1923)] by introducing the protecting group there was obtained ethyl 6-(2-trimethylsilyl-ethoxymethoxy)-naphthalene-2-carboxylate as a light yellow oil, MS: 346 (M)⁺.

(n) Reduction of ethyl 6-(2-trimethylsilyl-ethoxymethoxy)-naphthalene-2-carboxylate gave [6-(2-trimethylsilyl-ethoxy-methoxy)-naphthalen-2-yl]-methanol as a colorless solid, MS: 304 (M)⁻.

(o) Chlorination of [6-(2-trimethylsilyl-ethoxy-methoxy)-naphthalen-2-yl]-methanol yielded 6-chloromethyl-1-(β-trimethylsilylethoxymethoxy)-naphthalene as a colorless oil, MS: 322, 324 (M)⁺.

(p) Alkylation of (3RS,4RS)-1-benzyl-4-(4-fluoro-phenyl)-piperidin-3-ol with 6-chloromethyl-β-(β-trimethylsilyl-ethoxymethoxy)-naphthalene yielded (3RS,4RS)-1-benzyl-3-(4-fluorophenyl)-3-[6-(2-trimethyl-silylethoxy-methoxy)-naphthalen-2-ylmethoxy]-piperidine as a light yellow oil, MS: 572 (M+H)⁺.

(q) Cleavage of the N-benzyl group from (3RS,4RS)-1-benzyl-3-(4-fluorophenyl)-3-[6-(2-trimethyl-silylethoxy-methoxy)-naphthalen-2-ylmethoxy]-piperidine with β-trimethylsilylethyl chloroformate gave β-trimethylsilylethyl (3RS,4RS)-4-(4-fluorophenyl)-3-[6-(2-trimethylsilylethoxy-methoxy)-naphthalen-2-yl-methoxy]-piperidine-1-carboxylate as a colorless oil, MS: 626 (M+H)⁺.

(r) Cleavage of the SEM group from β-trimethylsilylethyl (3RS,4RS)-4-(4-fluorophenyl)-3-[6-(2-trimethylsilylethoxy-methoxy)-naphthalen-2-yl-methoxy]-piperidine-1-carboxylate yielded β-trimethylsilylethyl (3RS,4RS)-4-(4-fluorophenyl)-3-(6-hydroxy-naphthalen-2-ylmethoxy)-piperidine-1-carboxylate as a colorless resin, MS: 495 (M)⁺.

(s) From 7-hydroxy-naphthalene-2-carboxylic acid [Bull. Soc. Chim. Fr., 573 (1952)] there was firstly obtained by esterification with methanol/sulfuric acid methyl 7-hydroxy-naphthalene-2-carboxylate as a colorless solid, MS: 202 (M)⁺. Introduction of the protecting group yielded methyl 7-(2-trimethylsilyl-ethoxymethoxy)-naphthalene-2-carboxylate as a light yellow oil, MS: 332 (M)⁺.

(t) Reduction of methyl 7-(2-trimethylsilyl-ethoxymethoxy)-naphthalene-2-carboxylate gave [7-(2-trimethylsilyl-ethoxy-methoxy)-naphthalen-2-yl]-methanol as a light yellow oil, MS: 304 (M)⁺.

(u) Chlorination of [7-(2-trimethylsilyl-ethoxy-methoxy)-naphthalen-2-yl]-methanol yielded 2-chloromethyl-7-(β-trimethyl-silylethoxymethoxy)-naphthalene as a light yellow oil, MS: 322, 324 (M)⁺.

(v) Alkylation of (3RS,4RS)-1-benzyl-4-(4-fluoro-phenyl)-piperidin-3-ol with 2-chloromethyl-7-(β-trimethyl-silylethoxy-methoxy)-naphthalene yielded (3RS,4RS)-1-benzyl-3-(4-fluoro-phenyl)-3-[7-(2-trimethylsilyl-ethoxymethoxy)-naphthalen-2-ylmethoxy]-piperidine as a light yellow oil, MS: 572 (M+H)⁺.

(w) Cleavage of the N-benzyl group from (3RS,4RS)-1-benzyl-3-(4-fluoro-phenyl)-3-[7-(2-trimethylsilyl-ethoxymethoxy)-naphthalen-2-ylmethoxy]-piperidine with P-trimethylsilylethyl chloroformate yielded β-trimethylsilylethyl (3RS,4RS)-4-(4-fluorophenyl)-3-[7-(2-trimethylsilyl-ethoxymethoxy)-naphthalen-2-ylmethoxy]-piperidine-1-carboxylate as a colorless oil, MS: 626 (M+H)⁺.

(x) Cleavage of the SEM group from β-trimethylsilylethyl (3RS,4RS)-4-(4-fluorophenyl)-3-[7-(2-trimethylsilyl-ethoxy-methoxy)-naphthalen-2-ylmethoxy]-piperdine-1-carboxylate yielded β-trimethylsilylethyl (3RS,4RS)-4-(4-fluorophenyl)-3-(7-hydroxy-naphthalen-2-ylmethoxy)-piperidine-1-carboxylate as a light yellow oil, MS: 495 (M)⁺.

(y) From 8-hydroxy-naphthalene-2-carboxylic acid [Bull. Soc. Chim. Fr., 857 (1953)] there was firstly obtained by esterification with methanol/sulfuric acid methyl 8-hydroxy-naphthalene-2-carboxylate as a light yellow solid, MS: 202 (M)⁺. Introduction of the protecting group yielded methyl 8-(2-trimethylsilyl-ethoxy-methoxy)-naphthalene-2-carboxylate as a colorless solid, MS: 274 [M-(C₂H₄+CH₂O)]⁺.

(z) Reduction of methyl 8-(2-trimethylsilyl-ethoxymethoxy)-naphthalene-2-carboxylate gave [8-(2-trimethylsilyl-ethoxy-methoxy)-naphthalen-2-yl]-methanol as a colorless oil, MS: 304 (M)⁺.

(aa) Chlorination of [8-(2-trimethylsilyl-ethoxymethoxy)-naphthalen-2-yl]-methanol yielded 2-chloromethyl-8-(2-trimethylsilylethoxy-methoxy)-naphthalene as a light yellow oil, MS: 322, 324 (M)⁺.

(bb) Alkylation of (3RS,4RS)-1-benzyl-4-(4-fluoro-phenyl)-piperidin-3-ol with 2-chloromethyl-8-(2-trimethylsilylethoxy-methoxy)-naphthalene yielded (3RS,4RS)-1-benzyl-3-(4-fluorophenyl)-3-[8-(2-trimethylsilylethoxy-methoxy)-naphthalen-2-ylmethoxy]-piperidine as a light yellow oil, MS: 572 (M+H)⁺.

(cc) Cleavage of the N-benzyl group from (3RS,4RS)-1-benzyl-3-(4-fluoro-phenyl)-3-[8-(2-trimethylsilylethoxy-methoxy)-naphthalen-2-ylmethoxy]-piperidine with β-trimethylsilylethyl chloroformate gave β-trimethylsilylethyl (3RS,4RS)-4-(4-fluorophenyl)-3-[8-(2-trimethylsilyl-ethoxymethoxy)-naphthalen-2-yl-methoxy]-piperidine-1-carboxylate as a colorless oil, MS: 626 (M+H)⁺.

(dd) Cleavage of the SEM group from β-trimethylsilylethyl 30 (3RS,4RS)-4-(4-fluorophenyl)-3-[8-(2-trimethylsilyl-ethoxymethoxy)-naphthalen-2-yl-methoxy]-piperidine-1-carboxylate yielded β-trimethylsilylethyl (3RS,4RS)-4-(4-fluorophenyl)-3-(8-hydroxy-naphthalen-2-ylmethoxy)-piperidine-1-carboxylate as a colorless oil, MS: 494 (M-H)⁻.

### Example 7

The following compounds were prepared in an analogous manner to that described in Examples 3 and 5:

### 1) (3RS,4RS)-4-(4-Fluorophenyl)-3-(3-hydroxy-naphthalen-2-ylmethoxy)-piperidine

In an analogous manner to that described in Examples 3 and 5 (g), by cleaving off the two protecting groups with methanolic hydrochloric acid from tert-butyl (3RS,4RS)-4-(4-fluorophenyl)-3-[3-(2-trimethylsilyl-ethoxymethoxy)-naphthalen-2-yl-methoxy]-piperidine-1-carboxylate there was obtained (3RS,4RS)-4-(4-fluorophenyl)-3-(3-hydroxy-naphthalen-2-yl-methoxy)-piperidine as a colorless solid, MS: 351 (M)⁺.

The tert-butyl (3RS,4RS)-4-(4-fluorophenyl)-3-[3-(2-trimethylsilyl-ethoxymethoxy)-naphthalen-2-ylmethoxy]-piperidine-1-carboxylate used as the starting material was prepared as follows:

(a) In an analogous manner to that described in Example 5 (a), from methyl 3-hydroxy-naphthalene-2-carboxylate by introducing the protecting group there was obtained methyl 3-(2-trimethylsilyl-ethoxy-methoxy)-naphthalene-2-carboxylate as a light yellow oil, MS: 274 (M-(C₂H₄+CH₂O)]⁺.

(b) In an analogous manner to that described in Example 5(b), reduction of methyl 3-(2-trimethylsilyl-ethoxy-methoxy)-naphthalene-2-carboxylate gave [3-(2-trimethylsilylethoxy-methoxy)-naphthalen-2-yl]-methanol as a light yellow oil, MS: 304 (M)⁺.

(c) 400 mg (1.30 mmol) of [3-(2-trimethylsilylethoxy-methoxy)-naphthalen-2-yl]-methanol and 462 mg (1.81 mmol) of carbon tetrabromide were dissolved in 5 ml of absolute acetonitrile and the solution was cooled to 0° C. A solution of 446 mg (1.68 mmol) of triphenylphosphine in 6 ml of absolute acetonitrile was added dropwise thereto at 0° C. within 10 minutes and thereafter the mixture was stirred at 0° C. for a further 30 minutes. Subsequently, the solvent was distilled off under reduced pressure and, for purification, the crude product was chromatographed, without further working up, on silica gel using a 2:3 mixture of methylene chloride and hexane as the eluent. There were obtained 314 mg (65% of theory) of 2-bromomethyl-3-(2-trimethylsilylethoxymethoxy)-naphthalene as a light yellow oil; MS: 366, 368 (M)⁺.

(d) In an analogous manner to that described in Example 3 (c), by alkylating tert-butyl (3RS,4RS)-4-(4-fluorophenyl)-3-hydroxy-piperidine-1-carboxylate with 2-bromomethyl-3-(2-trimethyl-silylethoxy-methoxy)-naphthalene there was obtained tert-butyl (3RS,4RS)-4-(4-fluorophenyl)-3-[3-(2-trimethylsilyl-ethoxy-methoxy)-naphthalen-2-ylmethoxy]-piperidine-1-carboxylate as a light yellow oil; MS: 523 [M-(C₂H₄+CH₂O)]⁺.

### 2) (3RS,4RS)-4-(4-Fluorophenyl)-3-(1-methoxy-naphthalen-2-yl-methoxy)-piperidine

In an analogous manner to that described in Example 3, by cleavage of the BOC protecting group from tert-butyl (3RS,4RS)-4-(4-fluorophenyl)-3-[1-methoxy-naphthalen-2-ylmethoxy]-piperidine-1-carboxylate there was obtained (3RS,4RS)-4-(4-fluorophenyl)-3-(1-methoxy-naphthalen-2-yl-methoxy)-piperidine as a light yellow oil; MS: 365 (M)⁺.

The tert-butyl (3RS,4RS)-4-(4-fluorophenyl)-3-[1-methoxy-naphthalen-2-ylmethoxy]-piperidine-1-carboxylate used as the starting material was obtained as follows:

(e) In an analogous manner to that described in Example 5 (b), by reducing methyl 1-methoxy-naphthalene-2-carboxylate [J. Chem. Soc. 121 1657 (1922)] there was obtained [1-methoxy)-naphthalen-2-yl]-methanol as a colorless solid; MS: 188 (M)⁺.

(f) In an analogous manner to that described in Example 7 (c), by brominating [1-methoxy)-naphthalen-2-yl]-methanol there was obtained 2-bromomethyl-1-methoxy-naphthalene as a colorless solid; MS: 250, 252 (M)⁺.

(g) In an analogous manner to that described in Example 3 (c), by alkylating tert-butyl (3RS,4RS)-4-(4-fluorophenyl)-3-hydroxy-piperidine-1-carboxylate [Example 3 (b)] with 2-bromomethyl-1-methoxy-naphthalene there was obtained tert-butyl (3RS,4RS)-4-(4-fluorophenyl)-3-[1-methoxy-naphthalen-2-yl-methoxy]-piperidine-1-carboxylate as a colorless resin; MS: 465 (M)⁺.

### 3) (3RS,4RS)-4-(4-Fluorophenyl)-3-(3-methoxy-naphthalen-2-yl-methoxy)-piperidine

In an analogous manner to that described in Example 3, by cleavage of the BOC protecting group from tert-butyl (3RS,4RS)-4-(4-fluorophenyl)-3-[3-methoxy-naphthalen-2-ylmethoxy]-piperidine-1-carboxylate there was obtained (3RS,4RS)-4-(4-fluorophenyl)-3-(3-methoxy-naphthalen-2-yl-methoxy)-piperidine as a light yellow oil; MS: 365 (M)⁺.

The tert-butyl (3RS,4RS)-4-(4-fluorophenyl)-3-[3-methoxy-naphthalen-2-ylmethoxy]-piperidine-1-carboxylate used as the starting material was obtained as follows:

(h) In an analogous manner to that described in Example 5 (b), by reducing methyl 3-methoxy-naphthalene-2-carboxylate [J. Chem. Soc. 2351 (1950)] there was obtained [3-methoxy)-naphthalen-2-yl]-methanol as a colorless solid; MS: 188 (M)⁺.

(i) In an analogous manner to that described in Example 7 (c), by brominating [3-methoxy)-naphthalen-2-yl]-methanol there was obtained 2-bromomethyl-3-methoxy-naphthalene as a colorless solid; MS: 250, 252 (M)⁺.

(j) In an analogous manner to that described in Example 3 (c), by alkylating tert-butyl (3RS,4RS)-4-(4-fluorophenyl)-3-hydroxy-piperidine-1-carboxylate [Example 3 (b)] with 2-bromomethyl-3-methoxy-naphthalene there was obtained tert-butyl (3RS,4RS)-4-(4-fluorophenyl)-3-[3-methoxy-naphthalen-2-yl-methoxy]-piperidine-1-carboxylate as a colorless resin; MS: 465 (M)⁺.

### Example 8

(a) To 1.46 g of magnesium shavings, which had previously been covered with tetrahydrofuran, was added dropwise a solution of 12.06 g (60 mmol) of 5-bromo-benzo[1,3]dioxol in 30 ml of absolute tetrahydrofuran, followed by 11.35 g (60 mmol) of 1-benzyl-4-piperidone. The reaction mixture was stirred at 50° C. for 1 hour, then poured on to ice and ammonium chloride solution. The 4-benzo[1,3]dioxol-5-yl-1-benzyl-piperidin-4-ol formed was extracted with ethyl acetate and crystallized upon concentration of the solution. There were obtained 10.85 g (58% of theory) of white crystals; m.p.: 144° C.

(b) In an analogous manner to that described in Example 2(e), from 4-benzo[1,3]dioxol-5-yl-1-benzyl-piperidin-4-ol by catalytic hydrogenation at normal pressure within 4 hours there was obtained 4-benzo[1,3]dioxol-5-yl-piperidin-4-ol as a colorless solid in quantitative yield; MS: 221 (M)⁺.

(c) In an analogous manner to that described in Example 1(b), from 4-benzo[1,3]dioxol-5-yl-piperidin-4-ol by elimination there was obtained 4-benzo[1,3]dioxol-5-yl-1,2,3,6-tetrahydro-pyridine as a beige colored solid; MS: 203 (M)⁺.

(d) In an analogous manner to that described in Example 1(f), from 4-benzo[1,3]dioxol-5-yl-1,2,3,6-tetrahydro-pyridine by introducing the BOC group there was obtained tert-butyl 4-benzo[1,3]dioxol-5-yl-3,6-dihydro-2H-pyridine-1-carboxylate as a colorless oil; MS: 304 (M+H)⁺.

(e) In an analogous manner to that described in Example 1 (c), by hydroborating tert-butyl 4-benzo[1,3]dioxol-5-yl-3,6-dihydro-2H-pyridine-1-carboxylate there was obtained tert-butyl (3RS,4RS)-4-benzo[1,3]dioxol-5-yl-3-hydroxy-piperidine-1-carboxylate as white crystals; m.p.: 112° C.

(f) In an analogous manner to that described in Example 1(g), by alkylating tert-butyl (3RS,4RS)-4-benzo[1,3]dioxol-5-yl-3-hydroxy-piperidine-1-carboxylate with 2-bromomethylnaphthalene there was obtained tert-butyl (3RS,4RS)-4-benzo[1,3]dioxol-5-yl-3-(naphthalen-2-ylmethoxy)-piperidine-1-carboxylate as white crystals after crystallization from hexane; m.p.: 128-129° C.

(g) A solution of 190 mg (0.41 mmol) of tert-butyl (3RS,4RS)-4-benzo[1,3]dioxol-5-yl-3-(naphthalen-2-ylmethoxy)-piperidine-1-carboxylate in a mixture of 5 ml of methanol and 25% aqueous hydrochloric acid was heated under reflux for 1 hour. Subsequently, the solvent mixture was distilled off under reduced pressure. After recrystallization of the residue from a mixture of methanol and ether there were obtained 130 mg (73% of theory) of (3RS,4RS)-4-(1,3-benzodioxol-5-yl)-3-naphthalen-2-ylmethoxy-piperidine hydrochloride as a white powder; MS: 362 (M+H)⁺.

### Example 9

The following compounds were obtained in an analogous manner to that described in Example 8(g) by cleavage of the BOC group using acid:

1)—Pyridine-3-carboxylic acid (3RS,4RS)-2-(4-phenyl-piperidin-3-yloxymethyl)-benzylamide hydrochloride as a beige colored powder, MS: 402 (M+H)⁺, from tert-butyl (3RS,4RS)-4-phenyl-3-(2-{[(pyridine-3-carbonyl)-amino]-methyl}-benzyloxy)-piperidine-1-carboxylate;

2)—(3RS,4RS)-2-(4-[1,3]benzodioxol-5-yl-piperidin-3-yloxymethyl)-benzamide hydrochloride as a white powder, MS: 355 (M+H)⁺, from tert-butyl (3RS,4RS)-4-benzo[1,3]dioxol-5-yl-3-(2-carbamoyl-benzyloxy)-piperidine-1-carboxylate.

The BOC derivatives used as the starting materials were prepared as follows:

(a) In an analogous manner to that described in Example 1 (g), by alkylating tert-butyl (3RS,4RS)-3-hydroxy-4-phenyl-piperidine-1-carboxylate [Example 2(a)] with 2-bromomethylbenzonitrile there was obtained tert-butyl (3RS,4RS)-3-(2-cyano-benzyloxy)-4-phenyl-piperidine-1-carboxylate as a colorless oil; MS: 393 (M+H)⁺.

(b) 528 mg (1.35 mmol) of tert-butyl (3RS,4RS)-3-(2-cyano-benzyloxy)-4-phenyl-piperidine-1-carboxylate were reduced with 0.3 ml of borane-dimethyl sulfide complex in analogy to the process described by H. C. Brown et al. in Synthesis 1981, 605. There were obtained 480 mg (90% of theory) of tert-butyl (3RS,4RS)-3-(2-aminomethyl-benzyloxy)-4-phenyl-piperidine-1-carboxylate as a colorless solid; 397 (M+H)⁺.

(c) A solution of 150 mg (0.38 mmol) of tert-butyl (3RS,4RS)-3-(2-aminomethyl-benzyloxy)-4-phenyl-piperidine-1-carboxylate in 2 ml of methylene chloride was treated with 229 mg (2.26 mmol) of triethylamine, 139 mg (1.05 mmol) of nicotinic acid, 216 mg (1.13 mmol) of EDC and 10 mg (0.08 mmol) of 4-dimethylaminopyridine and the mixture was stirred at room temperature for 24 hours. Thereafter, the reaction solution was diluted with methylene chloride and washed with a saturated sodium hydrogen carbonate solution. The organic phase as separated, dried over sodium sulfate and evaporated under reduced pressure. The residue was chromatographed on silica gel using ethyl acetate as the eluent. There were obtained 100 mg (53% of theory) of tert-butyl (3RS,4RS)-4-phenyl-3-(2-{[(pyridine-3-carbonyl)-amino]-methyl}-benzyloxy)-piperidine-1-carboxylate as a colorless solid; MS: 502 (M+H)⁺.

(d) In an analogous manner to that described in Example 1(g), by alkylating tert-butyl (3RS,4RS)-4-benzo[1,3]dioxol-5-yl-3-(naphthalen-2-ylmethoxy)-piperidine-1-carboxylate [Example 8(f)] with 2-bromomethyl-benzonitrile there was obtained tert-butyl (3RS,4RS)-4-benzo[1,3]dioxol-5-yl-3-(2-cyano-benzyloxy)-piperidine-1-carboxylate as colorless crystals; MS: 455 (M+H)⁺.

(e) 0.5 ml of hydrogen peroxide (33%) and 0.2 ml of 2N sodium hydroxide solution were added to a solution of 236 mg (0.54 mmol) of tert-butyl (3RS,4RS)-4-benzo[1,3]dioxol-5-yl-3-(2-cyano-benzyloxy)-piperidine-1-carboxylate in 5 ml of methanol. The reaction solution was heated under reflux for 2 hours. Subsequently, the same amounts of hydrogen peroxide and sodium hydroxide solution were again added and the solution was heated for a further 2 hours. Thereafter, the solution was cooled and evaporated under reduced pressure. For purification, the residue was chromatographed on silica gel using a 9:1 mixture of methylene chloride and ether as the eluent. There were obtained 140 mg (57% of theory) of tert-butyl (3RS,4RS)-4-benzo[1,3]dioxol-5-yl-3-(2-carbamoyl-benzyloxy)-piperidine-1-carboxylate as a colorless solid; MS: 455 (M+H)⁺.

### Example 10

(a) In an analogous manner to that described in Example 1(g), by alkylating tert-butyl (3RS,4RS)-4-(4-fluorophenyl)-3-hydroxy-piperidine-1-carboxylate with 6-chloromethyl-2,3-dihydro-benzo[1,4]dioxin [Brit. Pat. 566732 (1943)] there was obtained tert-butyl (3RS,4RS)-3-(2,3-dihydro-benzo[1,4]dioxin-6-ylmethoxy)-4-(4-fluorophenyl)-piperidine-1-carboxylate as a colorless solid; MS: 444 (M+H)⁺;

(b) A solution of 280 mg (0.63 mmol) of tert-butyl (3RS,4RS)-3-(2,3-dihydro-benzo[1,4]dioxin-6-ylmethoxy)-4-(4-fluorophenyl)-piperidine-1-carboxylate in 5 ml of dry methylene chloride was treated with 808 mg (1.89 mmol) of anhydrous zinc bromide and the mixture was stirred at room temperature for 5 hours. Subsequently, the solvent was distilled off under reduced pressure, the residue was taken up in 10 ml of methanol, treated with 2 ml of 2N sodium hydroxide solution and the solid was separated. The filtrate was evaporated under reduced pressure and the residue was partitioned between methylene chloride and water. The organic phase was separated and evaporated under reduced pressure. There were obtained 220 mg (98% of theory) of (3RS,4RS)-3-(Z,3-dihydrobenzo[1,4]dioxin-6-yl-methoxy)-4-(4-fluorophenyl)-piperidine as a yellowish solid; MS: 344 (M+H)⁺.

### Example 11

In an analogous manner to that described in Example 10(b), from tert-butyl (3RS,4RS)-3-(benzo[b]furan-5-ylmethoxy)-4-(4-fluoro-phenyl)-piperidine-1-carboxylate there was obtained (3RS,4RS)-3-(benzo[b]furan-5-ylmethoxy)-4-(4-fluorophenyl)-piperidine as a colorless solid; MS: 326 (M+1)⁺;

The tert-butyl (3RS,4RS)-3-(benzo[b]furan-5-ylmethoxy)-4-(4-fluoro-phenyl)-piperidine-1-carboxylate used as the starting material was obtained as a colorless solid, MS: 426 (M+H)⁺, analogously to the procedure described in Example 1(g) by alkylating tert-butyl (3RS,4RS)-4-(4-fluorophenyl)-3-hydroxy-piperidine-1-carboxylate.

The 5-bromomethyl-benzo[b]furan used as the alkylating agent was prepared as follows:

By brominating 5-methyl-benzo[b]furan[Synth. Commun. 19, 257 (1989)] with N-bromosuccinimide in carbon tetrachloride in an analogous manner to the procedure for the preparation of 5-bromomethyl-benzo[b]thiophene [J. Med. Chem. 34(1), 65 (1991)] from 5-methyl-benzo[b]thiophene there was obtained 5-bromomethyl-benzo[b]furan as a light yellow solid; MS: 210, 212 (M)⁺.

### Example 12

(a) In an analogous manner to that described in Example 1(c), from 4-(4-chloro-phenyl)-1-methyl-1,2,3,6-tetrahydropyridine [U.S. Pat. No. 3,320,265] by hydroboration using borane in tetrahydrofuran there was obtained (3RS,4RS)-4-(4-chloro-phenyl)-1-methyl-piperidin-3-ol which, after recrystallization from a mixture of methylene chloride and hexane, formed colorless crystals of m.p.: 99-100° C.

(b) A solution of 1.12 g (5 mmol) of (3RS,4RS)-4-(4-chloro-phenyl)-1-methyl-piperidine-3-ol in 5 ml of tetrahydrofuran was added dropwise to a suspension of 0.264 g (5 mmol) of sodium hydride (50% dispersion in refined oil) in 8 ml of tetrahydrofuran and the mixture was stirred at 50° C. for 60 minutes. Subsequently, it was cooled to room temperature and treated with 1.10 g (5 mmol) of 2-bromomethyl-naphthalene in 5 ml of tetrahydrofuran. After 2 hours at 50° C. the reaction solution was poured into 60 ml of ice-water and extracted three times with 25 ml of ethyl acetate each time. The organic phases were washed with saturated sodium chloride solution, dried over magnesium sulfate, filtered and evaporated. The residue was chromatographed on silica gel using a 95:5 mixture of methylene chloride and ethanol as the eluent. There was obtained 0.539 (28% of theory) of (3RS,4RS)-4-(4-chloro-phenyl)-1-methyl-3-(naphthalen-2-ylmethoxy)-piperidine as a pale yellow oil; MS: 366 (M)⁺.

(c) A solution of 0.526 g (1.43 mmol) of (3RS,4RS)-4-(4-chloro-phenyl)-1-methyl-3-(naphthalen-2-ylmethoxy)-piperidine in 12 ml of toluene was treated with 100 mg of potassium carbonate and heated to 100°. Subsequently, 0.423 g (0.288 ml, 2 mmol) of 2,2,2-trichloroethyl chloroformate was added thereto and the mixture was stirred at 100° for 12 hours. The reaction solution was evaporated, taken up in 50 ml of ethyl acetate and washed with 20 ml of water and 20 ml of saturated sodium hydrogen carbonate solution. Drying over magnesium sulfate, filtration (sic) and evaporation yielded a colorless oil which was chromatographed on silica gel using a 3:2 mixture of hexane and ethyl acetate as the eluent. There was obtained 0.4269 (57% of theory) of 2,2,2-trichloroethyl 4-(4-chloro-phenyl)-3-(naphthalen-2-ylmethoxy)-piperidine-1-carboxylate as a colorless oil; Rf: 0.31 (silica gel, hexane/ethyl acetate: 3/2).

(d) A suspension of 0.420 g (0.8 mmol) of 2,2,2-trichloroethyl 4-(4-chloro-phenyl)-3-(naphthalen-2-ylmethoxy)-piperidine-1-carboxylate and 300 mg of zinc in 10 ml of acetic acid was stirred at room temperature for 12 hours. The reaction solution was diluted with 40 ml of water and extracted four times with 30 ml of methylene chloride. The organic phase was washed twice with 40 ml of 1N sodium hydroxide solution each time, dried over sodium sulfate, filtered and evaporated. The residue was chromatographed on silica gel using a 9:1 mixture of methylene chloride and methanol as the eluent. There was obtained 0.210 g (74% of theory) of (3RS,4RS)-4-(4-chloro-phenyl)-3-(naphthalen-2-ylmethoxy)-piperidine, MS: 210 (M-C₁ 1 H.sub.9)⁺, which was converted into the hydrochloride of m.p. 159-161° C. (dec.) with a solution of hydrogen chloride in methanol.

### Example 13

The following compounds were prepared in an analogous manner to that described in Example 12(b)-(d) by alkylation and subsequent cleavage of the N-methyl group:

1)—from (3RS,4RS)-4-(4-chloro-phenyl)-1-methyl-piperidin-3-ol and 1-bromomethylnaphthalene, (3RS,4RS)-4-(4-chloro-phenyl)-3-(naphthalene-1-ylmethoxy)-piperidine, MS: 210 (M-C₁ 1H.sub.9)⁺, which was converted with a solution of hydrogen chloride in ethanol into the hydrochloride of m.p. 210-213° C. (dec.).

2)—from (3RS,4RS)-4-(4-chloro-phenyl)-1-methyl-piperidin-3-ol and 1-bromomethyl-4-tert-butylbenzene, (3RS,4RS)-3-(4-tert.butyl-benzyloxy)-4-(4-chloro-phenyl)-piperidine, MS: 358 (M)⁺, which was converted with a solution of hydrogen chloride in ethanol into the hydrochloride of m.p. 164-166° C. (dec.).

3)—from (3RS,4RS)-4-(4-chloro-phenyl)-1-methyl-piperidin-3-ol and 5-chloromethyl-benzo[1.3]dioxol, (3RS,4RS)-3-(benzo-[1.3]dioxol-5-yl-methoxy)-4-(4-chloro-phenyl)-piperidine, MS: 210 (M-C₈H₇O₂)⁺, which was converted with methanesulfonic acid in a mixture of dioxan and water and subsequent lyophilization into the corresponding methanesulfonate; R_{f}: 0.45 (silica gel, methylene chloride/methanol: 9/1).

4)—from (3RS,4RS)-4-(4-chloro-phenyl)-1-methyl-piperidin-3-ol and 1,2-dichloro-4-chloromethylbenzene, (3RS,4RS)-4-(4-chloro-phenyl)-3-(3,4-dichlorobenzyloxy)-piperidine, MS: 370 (M)⁺, which was converted with a solution of hydrogen chloride in ethanol into the hydrochloride of m.p. 156-158° C. (dec.).

5)—from (3RS,4RS)-4-(4-chloro-phenyl)-1-methyl-piperidin-3-ol and 2,4-dichloro-1-chloromethylbenzene, (3RS,4RS)-4-(4-chloro-phenyl)-3-(2,4-dichloro-benzyloxy)-piperidine of m.p. 83-84° C.; MS: 370 (M)⁺.

6)—from (3RS,4RS)-4-(4-chloro-phenyl)-1-methyl-piperidin-3-ol and 1-chloro-4-chloromethylbenzene, (3RS,4RS)-3-(4-chlorobenzyloxy)-4-(4-chloro-phenyl)-piperidine, MS: 210 (M-C₇H₆Cl)⁺, which was converted with a solution of hydrogen chloride in ethanol into the hydrochloride of m.p. 128-130° C. (dec.).

7)—from (3RS,4RS)-4-(4-chloro-phenyl)-1-methyl-piperidin-3-ol and 1-chloromethyl-3-methoxy-benzene, (3RS,4RS)-(4-chloro-phenyl)-3-(2-methoxy-benzyloxy)-piperidine, MS: 332 (M)⁺, which was converted with a solution of hydrogen chloride in ethanol into the hydrochloride of m.p. 116-118° C. (dec.).

8)—from (3RS,4RS)-4-(4-chloro-phenyl)-1-methyl-piperidin-3-ol and 1-chloro-2-chloromethyl-benzene, (3RS,4RS)-3-(2-chlorobenzyloxy)-4-(4-chloro-phenyl)-piperidine, MS: 210 (M-C₇H₆Cl)⁺, which was converted with a solution of hydrogen chloride in ethanol into the hydrochloride of m.p. 145-147° C. (dec.).

9)—from (3RS,4RS)-4-(4-chloro-phenyl)-1-methyl-piperidin-3-ol and 4-chloromethyl-biphenyl, (3RS,4RS)-3-(biphenyl-4-ylmethoxy)-4-(4-chloro-phenyl)-piperidine, MS: 210 (M-C₁₃H₁₁)⁺, which was converted with a solution of hydrogen chloride in ethanol into the hydrochloride of m.p. 177-180° C. (dec.).

10)—from (3RS,4RS)-4-(4-chloro-phenyl)-1-methyl-piperidin-3-ol and 2-chloromethyl-quinoline, (3RS,4RS)-2-[4-(4-chloro-phenyl)-piperidin-3-yloxy-methyl]quinoline, MS: 353 (M)⁺ of m.p. 109-110° C.

11)—from (3RS,4RS)-4-(4-chloro-phenyl)-1-methyl-piperidin-3-ol and 3-chloromethyl-benzofuran [J. Am. Chem. Soc. 73, 4400 (1951)], (3RS,4RS)-3-(benzofuran-2-ylmethoxy)-4-(4-chloro-phenyl)-piperidine, MS: 341 (M)⁺, which was converted with a solution of hydrogen chloride in ethanol into the hydrochloride of m.p. 144-146° C. (dec.).

12)—from (3RS,4RS)-4-(4-chloro-phenyl)-1-methyl-piperidin-3-ol and 2-chloromethyl-benzo[b]thiophene [J. Am. Chem. Soc. 71, 2856 (1949)], (3RS,4RS)-3-(benzo[b]thiophen-2-ylmethoxy)-4-(4-chloro-phenyl)-piperidine, MS: 210 (M-C₈H₇S)⁺, which was converted with a solution of hydrogen chloride in ethanol into the hydrochloride of m.p. 141-144° C. (dec.).

13)—from (3RS,4RS)-4-(4-chloro-phenyl)-1-methyl-piperidin-3-ol and methyl 4′-bromomethyl-biphenyl-2-carboxylate [J. Med. Chem. 34, 2525 (1991)], methyl (3RS,4RS)-4′-[4-(4-chloro-phenyl)-piperidin-3-yloxy-methyl]-biphenyl-2-carboxylate, MS: 436 (M)⁺, which was converted with hydrochloric acid in ethanol into the hydrochloride of m.p. 95-99° C. (dec.).

14)—from (3RS,4RS)-4-(4-chloro-phenyl)-1-methyl-piperidin-3-ol and 3-chloromethyl-pyridine [J. Am. Chem. Soc. 77, 1054 (1955)], (3RS,4RS)-3-[4-(4-chloro-phenyl-piperidin-3-yloxymethyl]-pyridine, MS: 303 (M)⁺, which was converted with a solution of hydrogen chloride in ethanol into the hydrochloride of m.p. 78-81° C. (dec.).

15)—from (3RS,4RS)-4-(4-chloro-phenyl)-1-methyl-piperidin-3-ol and 6-chloromethyl-1,1,4,4,-tetramethyl-1,2,3,4-tetrahydronaphthalene, (3RS,4RS)-4-(4-chloro-phenyl)-3-(5,5,8,8,-tetramethyl-5,6,7,8-tetrahydronaphthalene-2-ylmethoxy)-piperidine, MS: 412 (M)⁺, which was converted with a solution of hydrogen chloride in ethanol into the hydrochloride of m.p. 118-121° C. (dec.).

16)—from (3RS,4RS)-4-(3-chloro-phenyl)-1-methyl-piperidin-3-ol [U.S. Pat. No. 4,132,710 (1976)] and 4-methoxybenzyl chloride, (3RS,4RS)-3-(4-methoxy-benzyloxy)-4-(3-chloro-phenyl)-piperidine; MS: 332 (M)⁺.

### Example 14

The following compounds were prepared in analogy to the procedure described in Example 1 (e) by cleavage of the 2-trimethylsilyl-ethoxycarbonyl group with tetrabutylammonium fluoride in tetrahydrofuran:

1)—(3RS,4RS)-4-(4-fluorophenyl)-3-(4-methoxy-naphthalen-2-ylmethoxy)-piperidine as a colorless solid, MS: 365 (M)⁺, from β-trimethyl-silylethyl (3RS,4RS)-4-(4-fluorophenyl)-3-(4-methoxy-naphthalen-2-ylmethoxy)-piperidine-1-carboxylate;

2)—(3RS,4RS)-4-(4-fluorophenyl)-3-(5-methoxy-naphthalen-2-ylmethoxy)-piperidine as a colorless solid, MS: 365 (M)⁺, from β-trimethyl-silylethyl (3RS,4RS)-4-(4-fluorophenyl)-3-(5-methoxy-naphthalen-2-ylmethoxy)-piperidine-1-carboxylate;

3)—(3RS,4RS)-4-(4-fluorophenyl)-3-(6-methoxy-naphthalen-2-ylmethoxy)-piperidine as a colorless solid, MS: 365 (M)⁺, from β-trimethyl-silylethyl (3RS,4RS)-4-(4-fluorophenyl)-3-(6-methoxy-naphthalen-2-ylmethoxy)-piperidine-1-carboxylate;

4)—(3RS,4RS)-4-(4-fluorophenyl)-3-(7-methoxy-naphthalen-2-ylmethoxy)-piperidine as a colorless solid, MS: 365 (M)⁺, from β-trimethyl-silylethyl (3RS,4RS)-4-(4-fluorophenyl)-3-(7-methoxy-naphthalen-2-ylmethoxy)-piperidine-1-carboxylate;

5)—(3RS,4RS)-4-(4-fluorophenyl)-3-(8-methoxy-naphthalen-2-ylmethoxy)-piperidine as a colorless resin, MS: 366 (M+H)⁺, from β-trimethyl-silylethyl (3RS,4RS)-4-(4-fluorophenyl)-3-(8-methoxy-naphthalen-2-ylmethoxy)-piperidine-1-carboxylate;

6)—(3RS,4RS)-4-(4-fluorophenyl)-3-[4-(pyridin-2-ylmethoxy)-naphthalen-2-yl methoxy]-piperidine as a colorless solid, MS: 442 (M)⁺, from β-trimethyl-silylethyl (3RS,4RS)-4-(4-fluorophenyl)-3-[4-(pyridin-2-ylmethoxy)-naphthalen-2-ylmethoxy]-piperidine-1-carboxylate;

7)—(3RS,4RS)-4-(4-fluorophenyl)-3-[4-(pyridin-3-ylmethoxy)-naphthalen-2-yl methoxy]-piperidine as a colorless solid, MS: 443 (M+H)⁺, from β-trimethyl-silylethyl (3RS,4RS)-4-(4-fluorophenyl)-3-[4-(pyridin-3-ylmethoxy)-naphthalen-2-ylmethoxy]-piperidine-1-carboxylate;

8)—(3RS,4RS)-4-(4-fluorophenyl)-3-[4-(pyridin-4-ylmethoxy)-naphthalen-2-yl methoxy]-piperidine as a colorless solid, MS: 442 (M)⁺, from β-trimethyl-silylethyl (3RS,4RS)-4-(4-fluorophenyl)-3-[4-(pyridin-4-ylmethoxy)-naphthalen-2-ylmethoxy]-piperidine-1-carboxylate;

9)—(3RS,4RS)-3-(4-allyloxy-naphthalen-2-ylmethoxy)-4-(4-fluorophenyl)-piperidine as a colorless solid, MS: 391 (M)⁺, from β-trimethyl-silylethyl (3RS,4RS)-3-(4-allyloxy-naphthalen-2-ylmethoxy)-4-(4-fluorophenyl)-piperidine-1-carboxylate;

10)—(3RS,4RS)-3-(6-allyloxy-naphthalen-2-ylmethoxy)-4-(4-fluorophenyl)-piperidine as a colorless solid, MS: 391 (M)⁺, from 5-trimethyl-silylethyl (3RS,4RS)-3-(6-allyloxy-naphthalen-2-ylmethoxy)-4-(4-fluorophenyl)-piperidine-1-carboxylate;

11)—(3RS,4RS)-4-(4-fluorophenyl)-3-(4-isobutyloxy-naphthalen-2-ylmethoxy)-piperidine as a colorless solid, MS: 407 (M)⁺, from β-trimethyl-silylethyl (3RS,4RS)-4-(4-fluorophenyl)-3-(4-isobutoxy-naphthalen-2-ylmethoxy)-piperidine-1-carboxylate;

12)—(3RS,4RS)-3-(1-benzyloxy-naphthalen-2-ylmethoxy)-4-(4-fluorophenyl)-piperidine as a light yellow oil, MS: 441 (M)⁺, from β-trimethyl-silylethyl (3RS,4RS)-3-(1-benzyloxy-naphthalen-2-ylmethoxy)-4-(4-fluorophenyl)-piperidine-1-carboxylate;

13)—(3RS,4RS)-3-(4-benzyloxy-naphthalen-2-ylmethoxy)-4-(4-fluorophenyl)-piperidine as a colorless solid, MS: 441 (M)⁺, from β-trimethyl-silylethyl (3RS,4RS)-3-(4-benzyloxy-naphthalen-2-ylmethoxy)-4-(4-fluorophenyl)-piperidine-1-carboxylate;

14)—(3RS,4RS)-3-(5-benzyloxy-naphthalen-2-ylmethoxy)-4-(4-fluorophenyl)-piperidine as a light yellow oil, MS: 442 (M+H)⁺, from β-trimethyl-silylethyl (3RS,4RS)-3-(5-benzyloxy-naphthalen-2-ylmethoxy)-4-(4-fluorophenyl)-piperidine-1-carboxylate;

15)—(3RS,4RS)-3-(7-benzyloxy-naphthalen-2-ylmethoxy)-4-(4-fluorophenyl)-piperidine as a colorless solid, MS: 441 (M)⁺, from β-trimethyl-silylethyl (3RS,4RS)-3-(7-benzyloxy-naphthalen-2-ylmethoxy)-4-(4-fluorophenyl)-piperidine-1-carboxylate;

16)—(3RS,4RS)-3-(8-benzyloxy-naphthalen-2-ylmethoxy)-4-(4-fluorophenyl)-piperidine as a light yellow resin, MS: 442 (M+H)⁺, from β-trimethyl-silylethyl (3RS,4RS)-3-(8-benzyloxy-naphthalen-2-ylmethoxy)-4-(4-fluorophenyl)-piperidine-1-carboxylate;

17)—(3RS,4RS)-4-(4-fluorophenyl)-3-[4-(2-methoxy-ethoxy)-naphthalen-2-ylmethoxy]-piperidine as a colorless solid, MS: 410 (M+H)⁺, from β-trimethyl-silylethyl (3RS,4RS)-4-(4-fluorophenyl)-3-[4-(2-methoxy-ethoxy)-naphthalen-2-ylmethoxy]-piperidine-1-carboxylate;

18)—4-(4-fluorophenyl)-3-[4-(3-methoxy-propoxy)-naphthalen-2-ylmethoxy]-piperidine as a light yellow solid, MS: 424 (M+H)⁺, from β-trimethyl-silylethyl 4-(4-fluorophenyl)-3-[4-(3-methoxy-propoxy)-naphthalen-2-ylmethoxy]-piperidine-1-carboxylate;

19)—(3RS,4RS) 3-(4-butoxy-naphthalen-2-ylmethoxy)-4-(4-fluorophenyl)-piperidine as a colorless solid, MS: 408 (M+H)⁺, from β-trimethyl-silylethyl (3RS,4RS) 3-(4-butoxy-naphthalen-2-ylmethoxy)-4-(4-fluorophenyl)-piperidine-1-carboxylate;

20)—(3RS,4RS)-4-(4-fluorophenyl)-3-(4-(2-methoxy-benzyloxy)-naphthalen-2-ylmethoxy)-piperidine as a colorless resin, MS: 472 (M+H)⁺, from β-trimethyl-silylethyl (3RS,4RS)-4-(4-fluorophenyl)-3-(4-(2-methoxy-benzyloxy)-naphthalen-2-ylmethoxy)-piperidine-1-carboxylate;

21)—(3RS,4RS)-4-(4-fluorophenyl)-3-(4-(3-methoxy-benzyloxy)-naphthalen-2-ylmethoxy)-piperidine as a colorless solid, MS: 471 (M)⁺, from β-trimethyl-silylethyl (3RS,4RS)-4-(4-fluorophenyl)-3-(4-(3-methoxy-benzyloxy)-naphthalen-2-ylmethoxy)-piperidine-1-carboxylate;

22)—(3RS,4RS)-4-(4-fluorophenyl)-3-(4-(2-methoxy-benzyloxy)-naphthalen-2-ylmethoxy)-piperidine as a colorless oil, MS: 471 (M)⁺, from β-trimethyl-silylethyl (3RS,4RS)-4-(4-fluorophenyl)-3-(4-methoxy-benzyloxy)-naphthalen-2-ylmethoxy)-piperidine-1-carboxylate;

23)—(3RS,4RS)-4-(4-fluorophenyl)-3-(1-phenethyloxy-naphthalen-2-ylmethoxy)-piperidine as a light yellow oil, MS: 456 (M+H)⁺, from β-trimethyl-silylethyl (3RS,4RS)-4-(4-fluorophenyl)-3-(1-phenethyloxy-naphthalen-2-ylmethoxy)-piperidine-1-carboxylate;

24)—(3RS,4RS)-4-(4-fluorophenyl)-3-(4-phenethyloxy-naphthalen-2-ylmethoxy)-piperidine as a colorless solid, MS: 456 (M+H)⁺, from 5-trimethyl-silylethyl (3RS,4RS)-4-(4-fluorophenyl)-3-(4-phenethyloxy-naphthalen-2-ylmethoxy)-piperidine-1-carboxylate;

25)—(3RS,4RS)-3-[4-(2-[1,3]dioxolan-2-yl-ethoxy)-naphthalen-2-ylmethoxy]-4-(4-fluorophenyl)-piperidine as a light yellow oil, MS: 451 (M)⁺, from β-trimethyl-silylethyl (3RS,4RS)-3-[4-(2-[1,3]dioxolan-2-yl-ethoxy)-naphthalen-2-ylmethoxy]-4-(4-fluorophenyl)-piperidine-1-carboxylate;

26)—(3RS,4RS)-3-[1-(2-[1,3]dioxolan-2-yl-ethoxy)-naphthalen-2-ylmethoxy]-4-(4-fluorophenyl)-piperidine as a light yellow oil, MS: 452 (M+H)⁺, from β-trimethyl-silylethyl (3RS,4RS)-3-[1-(2-[1,3]dioxolan-2-yl-ethoxy)-naphthalen-2-ylmethoxy]-4-(4-fluorophenyl)-piperidine-1-carboxylate;

27)—(3RS,4RS)-3-[4-(benzo[1,3]dioxol-5-ylmethoxy)-naphthalen-2-ylmethoxy]-4-(4-fluorophenyl)-piperidine as a colorless resin, MS: 485 (M)⁺, from β-trimethyl-silylethyl 3RS,4RS)-3-[4-(benzo[1,3]dioxol-5-ylmethoxy)-naphthalen-2-ylmethoxy]-4-(4-fluorophenyl)-piperidine-1-carboxylate;

28)—(3RS,4RS)-3-[4-(2-cyclopropyl-ethoxy)-naphthalen-2-ylmethoxy]-4-(4-fluorophenyl)-piperidine as a colorless solid, MS: 419 (M)⁺, from β-trimethyl-silylethyl (3RS,4RS)-3-[4-(2-cyclopropyl-ethoxy)-naphthalen-2-ylmethoxy]-4-(4-fluorophenyl)-piperidine-1-carboxylate;

29)—(3RS,4RS)-4-(4-fluorophenyl)-3-[4-(2-hydroxy-ethoxy)-naphthalen-2-ylmethoxy]-piperidine as a colorless solid, MS: 395 (M)⁺, from β-trimethyl-silylethyl (3RS,4RS)-4-(4-fluorophenyl)-3-[4-(2-hydroxy-ethoxy)-naphthalen-2-ylmethoxy]-piperidine-1-carboxylate;

The compounds used as starting materials were prepared as follows:

(a) 99 mg (0.20 mmol) of β-trimethyl-silylethyl (3RS,4RS)-4-(4-fluorophenyl)-3-(4-hydroxy-naphthalen-2-ylmethoxy)-piperidine-1-carboxylate were dissolved in 1 ml of dimethylformamide, treated with 69 mg (0.50 mmol) of anhydrous potassium carbonate and 19 μl (43 mg, 0.30 mmol) of methyl iodide and stirred at room temperature for 4 hours. Subsequently, the mixture was partitioned between methylene chloride and water, the organic phase was dried over magnesium sulfate and finally the solvent was distilled off under reduced pressure. For purification, the crude product was chromatographed on silica gel with a 4:1 mixture of hexane and methylene chloride as the eluent. There were obtained 85 mg (83% of theory) of p-trimethyl-silylethyl (3RS,4RS)-4-(4-fluorophenyl)-3-(4-methoxy-naphthalen-2-ylmethoxy)-piperidine-1-carboxylate as a light yellow oil; MS: 509 (M)⁺.

The following compounds were prepared in an analogous manner to the previously described procedure:

from β-trimethylsilylethyl (3RS,4RS)-4-(4-fluorophenyl)-3-(5-hydroxy-naphthalen-2-ylmethoxy)-piperidine-1-carboxylate and methyl iodide, β-trimethylsilylethyl (3RS,4RS)-4-(4-fluorophenyl)-3-(5-methoxy-naphthalen-2-ylmethoxy)-piperidine-1-carboxylate, which was used in crude form in the next step;

from β-trimethylsilylethyl (3RS,4RS)-4-(4-fluorophenyl)-3-(6-hydroxy-naphthalen-2-ylmethoxy)-piperidine-1-carboxylate and methyl iodide, β-trimethylsilylethyl (3RS,4RS)-4-(4-fluorophenyl)-3-(6-methoxy-naphthalen-2-ylmethoxy)-piperidine-1-carboxylate as a light yellow oil; MS: 509 (M)⁺;

from β-trimethylsilylethyl (3RS,4RS)-4-(4-fluorophenyl)-3-(7-hydroxy-naphthalen-2-ylmethoxy)-piperidine-1-carboxylate and methyl iodide, β-trimethylsilylethyl (3RS,4RS)-4-(4-fluorophenyl)-3-(7-methoxy-naphthalen-2-ylmethoxy)-piperidine-1-carboxylate, which was used in crude form in the next step;

from β-trimethylsilylethyl (3RS,4RS)-4-(4-fluorophenyl)-3-(8-hydroxy-naphthalen-2-ylmethoxy)-piperidine-1-carboxylate and methyl iodide, β-trimethylsilylethyl (3RS,4RS)-4-(4-fluorophenyl)-3-(8-methoxy-naphthalen-2-ylmethoxy)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and 2-pyridylmethyl chloride, β-trimethylsilylethyl (3RS,4RS)-4-(4-fluorophenyl)-3-[4-(pyridin-2-ylmethoxy)-naphthalen-2-yl-methoxy]-piperidine-1-carboxylate as a light yellow oil; MS: 587 (M+H)⁺;

from β-trimethylsilylethyl (3RS,4RS)-4-(4-fluorophenyl)-3-(4-hydroxy-naphthalen-2-ylmethoxy)-piperidine-1-carboxylate and 3-pyridylmethyl chloride, β-trimethylsilylethyl (3RS,4RS)-4-(4-fluorophenyl)-3-[4-(pyridin-3-ylmethoxy)-naphthalen-2-yl-methoxy]-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and 4-pyridylmethyl chloride, β-trimethylsilylethyl (3RS,4RS)-4-(4-fluorophenyl)-3-[4-(pyridin-4-ylmethoxy)-naphthalen-2-ylmethoxy]-piperidine-1-carboxylate as a light yellow oil; MS: 587 (M+H)⁺;

from β-trimethylsilylethyl (3RS,4RS)-4-(4-fluorophenyl)-3-(4-hydroxy-naphthalen-2-ylmethoxy)-piperidine-1-carboxylate and allyl bromide, β-trimethylsilylethyl (3RS,4RS)-3-(4-allyloxy-naphthalen-2-ylmethoxy)-4-(4-fluorophenyl)-piperidine-1-carboxylate, which was used in crude form in the next step;

from β-trimethylsilylethyl (3RS,4RS)-4-(4-fluorophenyl)-3-(6-hydroxy-naphthalen-2-ylmethoxy)-piperidine-1-carboxylate and allyl bromide, β-trimethylsilylethyl (3RS,4RS)-3-(6-allyloxy-naphthalen-2-ylmethoxy)-4-(4-fluorophenyl)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and iso-butyl bromide, β-trimethylsilylethyl (3RS,4RS)-4-(4-fluorophenyl)-3-(4-isobutoxy-naphthalen-2-ylmethoxy)-piperidine-1-carboxylate, which was used in crude form in the next step;

from β-trimethylsilylethyl (3RS,4RS)-4-(4-fluorophenyl)-3-(1-hydroxy-naphthalen-2-ylmethoxy)-piperidine-1-carboxylate and benzyl bromide, p-trimethylsilylethyl (3RS,4RS)-4-(4-fluorophenyl)-3-(1-benzyloxy-naphthalen-2-ylmethoxy)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and benzyl bromide, 13-trimethylsilylethyl (3RS,4RS)-3-(4-benzyloxy-naphthalen-2-ylmethoxy)-4-(4-fluorophenyl)-piperidine-1-carboxylate as a light yellow oil; MS: 586 (M+H)⁺;

from β-trimethylsilylethyl (3RS,4RS)-4-(4-fluorophenyl)-3-(7-hydroxy-naphthalen-2-ylmethoxy)-piperidine-1-carboxylate and benzyl bromide, β-trimethylsilylethyl (3RS,4RS)-3-(7-benzyloxy-naphthalen-2-ylmethoxy)-4-(4-fluorophenyl)-piperidine-1-carboxylate as a light yellow oil; MS: 585 (M)⁺;

from β-trimethylsilylethyl (3RS,4RS)-4-(4-fluorophenyl)-3-(5-hydroxy-naphthalen-2-ylmethoxy)-piperidine-1-carboxylate and benzyl bromide, 2-trimethylsilanyl-ethyl (3RS,4RS)-3-(5-benzyloxy-naphthalen-2-ylmethoxy)-4-(4-fluoro-phenyl)-piperidine-1-carboxylate as a colorless oil; MS: 585 (M)⁺;

from β-trimethylsilylethyl (3RS,4RS)-4-(4-fluorophenyl)-3-(8-hydroxy-naphthalen-2-ylmethoxy)-piperidine-1-carboxylate and benzyl bromide, β-trimethylsilylethyl (3RS,4RS)-3-(8-benzyloxy-naphthalen-2-ylmethoxy)-4-(4-fluorophenyl)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and 2-methoxyethyl bromide, 2-trimethylsilanyl-ethyl (3RS,4RS)-4-(4-fluoro-phenyl)-3-[4-(2-methoxy-ethoxy)-naphthalen-2-ylmethoxy]-piperidine-1-carboxylate, which was used in crude form in the next is step;

from β-trimethylsilylethyl (3RS,4RS)-4-(4-fluorophenyl)-3-(4-hydroxy-naphthalen-2-ylmethoxy)-piperidine-1-carboxylate and 3-methoxypropyl chloride [J. Org. Chem. 16, 704 (1951)], 2-trimethylsilanyl-ethyl (3RS,4RS)-4-(4-fluoro-phenyl)-3-[4-(3-methoxy-propoxy)-naphthalen-2-ylmethoxy]-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and butyl bromide, 2-trimethylsilanyl-ethyl (3RS,4RS)-3-(4-butyl-naphthalen-2-ylmethoxy)-4-(4-fluoro-phenyl)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and 2-methoxybenzyl chloride, 2-trimethylsilanyl-ethyl (3RS,4RS)-4-(4-fluoro-phenyl)-3-[4-(2-methoxy-benzyloxy)-naphthalen-2-ylmethoxy)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and 3-methoxybenzyl chloride, 2-trimethylsilanyl-ethyl (3RS,4RS)-4-(4-fluoro-phenyl)-3-[4-(3-methoxy-benzyloxy)-naphthalen-2-ylmethoxy)-piperidine-1-carboxylate as a light yellow oil; MS: 615 (M)⁺;

from β-trimethylsilylethyl (3RS,4RS)-4-(4-fluorophenyl)-3-(4-hydroxy-naphthalen-2-ylmethoxy)-piperidine-1-carboxylate and 4-methoxybenzyl chloride, 2-trimethylsilanyl-ethyl (3RS,4RS)-4-(4-fluoro-phenyl)-3-[4-(4-methoxy-benzyloxy)-naphthalen-2-ylmethoxy)-piperidine-1-carboxylate as a light yellow oil; MS: 616 (M+H)⁺;

from β-trimethylsilylethyl (3RS,4RS)-4-(4-fluorophenyl)-3-(4-hydroxy-naphthalen-2-ylmethoxy)-piperidine-1-carboxylate and phenethyl bromide, β-trimethylsilylethyl (3RS,4RS)-4-(4-fluorophenyl)-3-(4-phenethyloxy-naphthalen-2-ylmethoxy)-piperidine-1-carboxylate, which was used in crude form in the next step;

from β-trimethylsilylethyl (3RS,4RS)-4-(4-fluorophenyl)-3-(1-hydroxy-naphthalen-2-ylmethoxy)-piperidine-1-carboxylate and phenethyl bromide, 2-trimethylsilanyl-ethyl (3RS,4RS)-4-(4-fluorophenyl)-3-(1-phenethyloxy-naphthalen-2-ylmethoxy)-piperidine-1-carboxylate as a light yellow oil; MS: 600 (M+H)⁺;

from β-trimethylsilylethyl (3RS,4RS)-4-(4-fluorophenyl)-3-(4-hydroxy-naphthalen-2-ylmethoxy)-piperidine-1-carboxylate and 2-(2-bromoethyl)-1,3-dioxolan, 2-trimethylsilanyl-ethyl (3RS,4RS)-3-[4-(2-[1,3]dioxolan-2-yl-ethoxy)-naphthalen-2-ylmethoxy]-4-(4-fluorophenyl)-piperidine-1-carboxylate, which was used in crude form in the next step;

from β-trimethylsilylethyl (3RS,4RS)-4-(4-fluorophenyl)-3-(1-hydroxy-naphthalen-2-ylmethoxy)-piperidine-1-carboxylate and 2-(2-bromoethyl)-1,3-dioxolan, β-trimethylsilylethyl (3RS,4RS)-3-[1-(2-[1,3]dioxolan-2-yl-ethoxy)-naphthalen-2-ylmethoxy]-4-(4-fluorophenyl)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and 3,4-methylenedioxybenzyl chloride, β-trimethyl-silylethyl (3RS,4RS)-3-[4-(benzo[1,3]dioxol-5-ylmethoxy)-naphthalen-2-ylmethoxy]-4-(4-fluorophenyl)-piperidine-1-carboxylate, which was used in crude form in the next step;

from β-trimethylsilylethyl (3RS,4RS)-4-(4-fluorophenyl)-3-(4-hydroxy-naphthalen-2-ylmethoxy)-piperidine-1-carboxylate and 2-cyclopropyl-ethyl chloride [Justus Liebigs Ann. Chem. 759, 132 (1972)], β-trimethylsilylethyl (3RS,4RS)-3-[4-(2-cyclopropyl-ethoxy)-naphthalen-2-ylmethoxy]-4-(4-fluorophenyl)-piperidine-1-carboxylate as a light pink colored oil; MS: 564 (M+H)⁺;

from β-trimethylsilylethyl (3RS,4RS)-4-(4-fluorophenyl)-3-(4-hydroxy-naphthalen-2-ylmethoxy)-piperidine-1-carboxylate and 2-(2-bromoethoxy)-tetrahydropyran, a mixture of β-trimethylsilanylethyl (3RS,4RS)-4-(4-fluorophenyl)-3-{4-[2-[(RS)- and (SR)-tetrahydro-pyran-2-yloxy]-ethoxy]-naphthalen-2-ylmethoxy}-piperidine-1-carboxylate as a colorless oil; MS: 624 (M+H)⁺.

Subsequent cleavage of the THP protecting group with a 1M solution of hydrogen chloride in methanol (10 minutes, room temperature) yielded β-trimethyl-silylethyl (3RS,4RS)-4-(4-fluorophenyl)-3-[4-(2-hydroxy-ethoxy)-naphthalen-2-ylmethoxy]-piperidine-1-carboxylate as a colorless solid; MS: 540 (M+H)⁺.

### Example 15

(a) A solution of 63 mg (0.116 mmol) of β-trimethyl-silylethyl (3RS,4RS)-4-(4-fluorophenyl)-3-[4-(2-hydroxy-ethoxy)-naphthalen-2-ylmethoxy]-piperidine-1-carboxylate in 2 ml of methylene chloride was treated with 38 mg (0.58 mmol) of sodium cyanate. 44 μl (67 mg, 0.58 mmol) of trifluoroacetic acid were added to this suspension at 0° C. and the reaction mixture was stirred at room temperature for 2 hours. Subsequently, the mixture was partitioned between methylene chloride and a 5% sodium hydrogen carbonate solution, the organic phase was dried over magnesium sulfate and finally the solvent was distilled off under reduced pressure. The crude β-trimethyl-silylethyl (3RS,4RS)-3-[4-(2-carbamoyloxy-ethoxy)-naphthalen-2-ylmethoxy]-4-(4-fluorophenyl)-piperidine-1-carboxylate obtained was used in the following step without further purification and characterization.

(b) From the crude β-trimethyl-silylethyl (3RS,4RS)-3-[4-(2-carbamoyloxy-ethoxy)-naphthalen-2-ylmethoxy]-4-(4-fluorophenyl)-piperidine-1-carboxylate there was obtained by cleavage of the protecting group with tetrabutylammonium fluoride in tetrahydrofuran in analogy to the procedure described in Example 1(e) (3RS,4RS)-3-[4-(2-carbamoyloxy-ethoxy)-naphthalen-2-ylmethoxy]-4-(4-fluorophenyl)-piperidine as a light yellow oil; MS: 438 (M)⁺.

### Example 16

The following compounds were obtained in an analogous manner to that described in Example 1(e) by cleavage of the protecting group with tetrabutylammonium fluoride in tetrahydrofuran:

1)—4-(4-Fluorophenyl)-3-[4-[2-(pyridin-2-ylcarbamoyloxy)-ethoxy]-naphthalen-2-ylmethoxy]-piperidine as a colorless solid, MS: 395 [M-(PyNCO)]⁺, from β-trimethyl-silylethyl 4-(4-fluorophenyl)-3-[4-[2-(pyridin-2-ylcarbamoyloxy)-ethoxy]-naphthalen-2-ylmethoxy]-piperidine-1-carboxylate;

2)—(3RS,4RS)-3-[4-(2-benzoyloxy-ethoxy)-naphthalen-2-ylmethoxy]-4-(4-fluorophenyl)-piperidine as a colorless resin, MS: 500 (M+H)⁺, from β-trimethyl-silylethyl (3RS,4RS)-3-[4-(2-benzoyloxyethoxy)-naphthalen-2-ylmethoxy]-4-(4-fluorophenyl)-piperidine-1-carboxylate.

The β-trimethyl-silylethyl carbamates used as the starting materials were obtained as follows:

(a) A solution of 54 mg (0.10 mmol) of β-trimethyl-silylethyl (3RS,4RS)-4-(4-fluorophenyl)-3-[4-(2-hydroxy-ethoxy)-naphthalen-2-ylmethoxy]-piperidine-1-carboxylate in 5 ml of toluene was treated with 30 mg (0.20 mmol) of 2-pyridylcarbonyl azide [Monatsh. Chem. 33, 397 (1912)] and 5 mg of 4-dimethylaminopyridine. The solution was heated to reflux under argon for 2 hours. The mixture was cooled and the solvent was removed under reduced pressure. The residue was partitioned between methylene chloride and saturated sodium chloride solution, the organic phase was dried over magnesium sulfate and finally the solvent was removed under reduced pressure. The crude product (103 mg) was purified by chromatography on silica gel with a 1:2 mixture of ethyl acetate and hexane as the eluent. There were obtained 65 mg (99% of theory) of β-trimethyl-silylethyl (3RS,4RS)-4-(4-fluorophenyl)-3-[4-[2-(pyridin-2-ylcarbamoyloxy)-ethoxy]-naphthalen-2-ylmethoxy]-piperidine-1-carboxylate as a colorless solid; MS: 660 (M+H)⁺.

(b) A solution of 108 mg (0.20 mmol) of β-trimethyl-silylethyl (3RS,4RS)-4-(4-fluorophenyl)-3-[4-(2-hydroxy-ethoxy)-naphthalen-2-ylmethoxy]-piperidine-1-carboxylate in 2 ml of methylene chloride was treated with 56 μl (41 mg, 0.40 mmol) of triethylamine. 36 μl (42 mg, 0.30 mmol) of benzoyl chloride were added thereto. The reaction mixture was stirred at room temperature for 6 hours and at 50° C. for one hour. Subsequently, the mixture was partitioned between methylene chloride and a 5% sodium hydrogen carbonate solution, the organic phase was dried over magnesium sulfate and finally the solvent was removed under reduced pressure. The crude β-trimethyl-silylethyl (3RS,4RS)-3-[4-(2-benzoyloxy-ethoxy)-naphthalen-2-ylmethoxy]-4-(4-fluorophenyl)-piperidine-1-carboxylate was used directly in the next step.

### Example 17

The following compound was obtained in an analogous manner to that described in Example 5:

The procedure was carried out as follows in analogy to Example 5(a)-(d):

(a) From methyl salicylate there was obtained by introduction of the SEM group methyl 2-(2-trimethylsilylethoxymethoxy)-benzoate as a colorless oil; MS 224 [M-(C₂H₄+CH₂O)]⁺.

(b) Reduction of methyl 2-(2-trimethylsilyl-ethoxymethoxy)-benzoate gave [2-(2-trimethylsilylethoxy-methoxy)-phenyl]-methanol as a light yellow oil; MS: 226 [M-(C₂H₄)]⁺.

(c) Chlorination of [2-(2-trimethylsilylethoxy-methoxy)-phenyl]-methanol yielded 1-chloromethyl-(2-trimethylsilyl-ethoxymethoxy)-benzene as a colorless oil; MS: 214, 216 [M-(C₂H₄+CH₂O)]⁺.

(d) Alkylation of β-trimethyl-silylethyl (3RS,4RS)-4-(4-fluoro-phenyl)-3-(4-hydroxy-naphthalen-2-ylmethoxy)-piperidine-1-carboxylic acid [Example 5 (g)] with 1-chloromethyl-(2-trimethylsilyl-ethoxymethoxy)-benzene yielded β-trimethylsilylethyl (3RS,4RS)-4-(4-fluorophenyl)-3-[4-[2-(2-trimethylsilyl-ethoxymethoxy)-benzyloxy]-naphthalen-2-ylmethoxy]-piperidine-1-carboxylate as a colorless oil; MS: 749 (M+NH₄)⁺;

(e) From β-trimethylsilylethyl (3RS,4RS)-4-(4-fluorophenyl)-3-[4-[2-(2-trimethylsilyl-ethoxymethoxy)-benzyloxy]-naphthalen-2-ylmethoxy]-piperidine-1-carboxylate by cleaving the β-trimethylsilylethyl carbamate with tetrabutylammonium fluoride in tetrahydrofuran in analogy to the procedure described in Example 5 there was obtained (3RS,4RS)-4-(4-fluorophenyl)-3-[4-[2-(2-trimethylsilyl-ethoxymethoxy)-benzyloxy]-naphthalen-2-ylmethoxy]-piperidine as a rose colored oil; MS: 588 (M+H)⁺.

(f) Cleavage of the SEM group from (3RS,4RS)-4-(4-fluorophenyl)-3-[4-[2-(2-trimethylsilyl-ethoxymethoxy)-benzyloxy]-naphthalen-2-ylmethoxy]-piperidine using a 2N solution of hydrogen chloride in methanol analogously to the procedure described in Example 5 (g) yielded (3RS,4RS)-4-(4-fluorophenyl)-3-[4-[2-hydroxy-benzyloxy]-naphthalen-2-ylmethoxy]-piperidine as a colorless resin; MS: 458 (M+H)⁺.

### Example 18

The following compounds were obtained in an analogous manner to that described in Example 12 (d) by cleavage of the 2,2,2-trichloroethyl carbamate:

1)—(3RS,4RS)-4-(2-Fluorophenyl)-3-(naphthalen-2-ylmethoxy)-piperidine as a light yellow oil, MS: 336 (M+H)⁺, from 2,2,2-trichloroethyl (3RS,4RS)-4-(2-fluorophenyl)-3-(naphthalen-2-ylmethoxy)-piperidine-1-carboxylate;

2)—(3RS,4RS)-4-(3-fluorophenyl)-3-(naphthalen-2-ylmethoxy)-piperidine as a light yellow oil, MS: 336 (M+H)⁺, from 2,2,2-trichloroethyl (3RS,4RS)-4-(3-fluorophenyl)-3-(naphthalen-2-ylmethoxy)-piperidine-1-carboxylate;

3)—(3RS,4RS)-4-(3-hydroxyphenyl)-3-(naphthalen-2-ylmethoxy)-piperidine as a beige solid, MS: 333 (M)⁺, from 2,2,2-trichloroethyl (3RS,4RS)-3-(naphthalen-2-ylmethoxy)-4-[3-(2,2,2-trichloro-ethoxycarbonyloxy)-phenyl]-piperidine-1-carboxylate.

The 2,2,2-trichloroethyl carbamates used as the starting materials were prepared as follows:

(a) The following procedure was carried out in an analogous manner to that described in Example 1 (a)-(c):

From 2-bromofluorobenzene and 1-benzyl-4-piperidone there was obtained 1-benzyl-4-(2-fluorophenyl)-piperidin-4-ol as a yellow oil; MS: 285 (M)⁺. Subsequent elimination yielded 1-benzyl-4-(2-fluorophenyl)-1,2,3,6-tetrahydro-pyridine as a light yellow oil; MS: 267 (M)⁺. Hydroboration subsequently gave (3RS,4RS)-1-benzyl-4-(2-fluorophenyl)-piperidin-3-ol as a colorless solid; MS: 285 (M)⁺.

(b) In an analogous manner to that described in Example 1 (g), by alkylating (3RS,4RS)-1-benzyl-4-(2-fluorophenyl)-piperidin-3-ol with 2-bromomethylnaphthalene there was obtained (3RS,4RS)-1-benzyl-4-(2-fluorophenyl)-3-(naphthalen-2-ylmethoxy)-piperidine as a yellow oil; MS: 284 (M-C₁₁H₉)⁺. By cleavage of the benzyl protecting group with 2,2,2-trichloroethyl chloroformate in an analogous manner to that described in Example 12 (c) there was obtained 2,2,2-trichloroethyl (3RS,4RS)-4-(2-fluorophenyl)-3-(naphthalen-2-ylmethoxy)-piperdine-1-carboxylate as a yellow oil; MS: 509 (M)⁺.

(c) The following procedure was carried out in an analogous manner to that described in Example 1 (a)-(c):

From 3-bromofluorobenzene and 1-benzyl-4-piperidone there was obtained 1-benzyl-4-(3-fluorophenyl)-piperidin-4-ol as a colorless solid; MS: 285 (M)⁺. Subsequent elimination yielded 1-benzyl-4-(3-fluorophenyl)-1,2,3,6-tetrahydro-pyridine as a light yellow oil; MS: 267 (M)⁺. Hydroboration subsequently gave (3RS,4RS)-1-benzyl-4-(3-fluorophenyl)-piperidin-3-ol as a colorless oil; MS: 285 (M)⁺.

(d) In an analogous manner to that described in Example 1 (g), by alkylating (3RS,4RS)-1-benzyl-4-(3-fluorophenyl)-piperidin-3-ol with 2-bromomethylnaphthalene there was obtained (3RS,4RS)-1-benzyl-4-(3-fluorophenyl)-3-(naphthalen-2-ylmethoxy)-piperidine as a colorless oil; MS: 426 (M+H)⁺. By cleavage of the benzyl protecting group with 2,2,2-trichloroethyl chloroformate in an analogous manner to that described in Example 12 (c) there was obtained 2,2,2-trichloroethyl (3RS,4RS)-4-(3-fluorophenyl)-3-(naphthalen-2-ylmethoxy)-piperidine-1-carboxylate as a yellow oil; MS: 510 (M+H)⁺.

(e) The following procedure was carried out in an analogous manner to that described in Example 1 (a)-(c):

From 3-benzyloxy-iodobenzene [J. Chem. Soc. 2857 (1932)] and 1-benzyl-4-piperidone there was obtained 1-benzyl-4-(3-benzyloxy-phenyl)-piperidin-4-ol as a light yellow oil; MS: 373 (M)⁺. Subsequent elimination yielded 1-benzyl-4-(3-benzyloxy-phenyl)-1,2,3,6-tetrahydro-pyridine as a colorless solid; MS: 355 (M)⁺. Hydroboration subsequently gave (3RS,4RS)-1-benzyl-4-(3-benzyloxy-phenyl)-piperidin-3-ol as a colorless solid; MS: 373 (M)⁺.

(f) In an analogous manner to that described in Example 1 (g), by alkylating (3RS,4RS)-1-benzyl-4-(3-benzyloxy-phenyl)-piperidin-3-ol with 2-bromomethylnaphthalene there was obtained (3RS,4RS)-1-benzyl-4-(3-benzyloxy-phenyl)-3-(naphthalen-2-ylmethoxy)-piperidine as a colorless resin; MS: 514 (M+H)⁺.

(g) A solution of 250 mg (0.487 mmol) of (3RS,4RS)-1-benzyl-4-(3-benzyloxyphenyl)-3-(naphthalen-2-ylmethoxy)-piperidine in 1.1 ml of methylene chloride was treated at room temperature with 247 .mu.1 (236 mg, 1.946 mmol, 4 eq.) of N,N-dimethylaniline and 195 mg (1.46 mmol, 3.0 eq.) of aluminium trichloride and stirred at room temperature for 2.5 hours. Subsequently, the mixture was partitioned between methylene chloride and 5% sodium hydrogen carbonate solution, the organic phase was dried over magnesium sulfate and finally the solvent was removed under reduced pressure. The crude product was purified by chromatography on silica gel with a 4:1 mixture of methylene chloride and hexane as the eluent. There were obtained 65 mg (32% of theory) of (3RS,4RS)-1-benzyl-4-(3-hydroxyphenyl)-3-(naphthalen-2-ylmethoxy)-piperidine as a beige colored solid; MS: 423 (M)⁺.

(h) In an analogous manner to that described in Example 12(c), by cleavage of the benzyl group with 2,2,2-trichloroethyl chloroformate there was obtained 2,2,2-trichloroethyl (3RS,4RS)-3-(naphthalen-2-ylmethoxy)-4-[3-(2,2,2-trichloroethoxycarbonyloxy)-phenyl]-piperidine-1-carboxylate, which was used directly as the crude product in the next step.

### Example 19

In analogy to the procedure described in Example 2(e), by catalytically hydrogenating (3RS,4RS)-1-benzyl-4-(3-fluorophenyl)-piperidin-3-ol there was obtained (3RS,4RS)-4-(3-fluorophenyl)-piperidin-3-ol as a colorless solid; MS: 196 (M+H)⁺. Introduction of the BOC group in an analogous manner to that described in Example 1(f) yielded tert-butyl (3RS,4RS)-4-(3-fluorophenyl)-3-hydroxy-piperidine-1-carboxylate as a colorless solid; MS: 296 (M+H)⁺. Subsequent alkylation with 4-benzyloxy-2-chloromethyl-naphthalene in analogy to the procedure described in Example 1(g) gave tert-butyl (3RS,4RS)-3-(4-benzyloxy-naphthalen-2-ylmethoxy)-4-(3-fluorophenyl)-piperidine-1-carboxylate as a colorless solid; MS: 541 (M)⁺. Cleavage of the BOC group using a solution of hydrogen chloride in methanol analogously to the procedure described in Example 1(h) finally led to (3RS,4RS)-3-(4-benzyloxy-naphthalen-2-ylmethoxy)-4-(3-fluorophenyl)-piperidine, which was obtained as a colorless solid; MS: 442 (M+H)⁺.

The 4-benzyloxy-2-chloromethyl-naphthalene used as the starting compound was obtained as follows:

(a) By alkylating ethyl 4-hydroxy-naphthalene-2-carboxylate [J. Agric. Chem Soc. Japan 24, 313 (1950)] with benzyl bromide in an analogous manner to that described in Example 14(a) there was obtained ethyl 4-benzyloxy-naphthalene-2-carboxylate as an almost colorless solid; MS: 216 (M)⁺.

(b) Reduction of ethyl 4-benzyloxy-naphthalene-2-carboxylate analogously to Example 5(b) yielded (4-benzyloxy-naphthalen-2-yl)-methanol as a colorless solid; MS: 264 (M)⁺.

(c) Chlorination of (4-benzyloxy-naphthalen-2-yl) [-methanol] using carbon tetrachloride analogously to Example 7(c) yielded 4-benzyloxy-2-chloromethyl-naphthalene as a colorless solid; MS: 282 (M)⁺.

### Example 20

The following compounds were obtained in an analogous manner to that described in Example 1(h) by cleavage of the BOC group using a solution of hydrogen chloride in methanol:

1)—(3RS,4RS)-4-(4-Cyano-phenyl)-3-(naphthalen-2-ylmethoxy)-piperidine as a light yellow solid, MS: 342 (M+H)⁺, from tert-butyl (3RS,4RS)-4-(4-cyano-phenyl)-3-(naphthalen-2-ylmethoxy)-piperidine-1-carboxylate;

2)—(3RS,4RS)-4-[4-(phenylsulfonylamino-methyl)-phenyl]-3-(naphthalen-2-yl methoxy)-piperidine as a colorless solid, MS: 485 (M-H)⁻, from tert-butyl 4-[4-(phenylsulfonylamino-methyl)-phenyl]-3-(naphthalen-2-ylmethoxy)-piperidine-1-carboxylate;

3)—(3RS,4RS)-4-[4-(4-methoxy-benzoylamino)-methyl]-phenyl-3-(naphthalen-2-ylmethoxy)-piperidine as a colorless resin, MS: 481 (M+H)⁺, from tert-butyl (3RS,4RS)-4-[4-(4-methoxy-benzoylamino)-methyl]-phenyl-3-(naphthalen-2-ylmethoxy)-piperidine-1-carboxylate;

4)—(3RS,4RS)-3-(naphthalen-2-ylmethoxy)-4-[4-(phenylacetyl-aminomethyl)-phenyl]-piperidine as a colorless resin, MS: 465 (M+H)⁺, from tert-butyl (3RS,4RS)-3-(naphthalen-2-ylmethoxy)-4-[4-(phenylacetylamino-methyl)-phenyl]-piperidine-1-carboxylate;

5)—(3RS,4RS)-4-[4-(benzoylamino-methyl)-phenyl]-3-(4-hydroxy-naphthalen-2-ylmethoxy)-piperidine as a light orange colored solid, MS: 467 (M+H)⁺, from tert-butyl (3RS,4RS)-4-[4-(benzoylamino-methyl)-phenyl]-3-[4-(2-trimethylsilyl-ethoxy-methoxy)-naphthalen-2-ylmethoxy]-piperidine-1-carboxylate by simultaneous cleavage of the BOC and SEM groups analogously to that described in Example 3 and 5 (g).

The BOC compounds used as the starting materials were prepared as follows:

(a) A suspension of 20 mg (0.30 mmol) of activated zinc powder, 76 mg (1.17 mmol) of potassium cyanide, 52 mg (0.20 mmol) of triphenylphosphine and 74 mg (0.10 mmol) of bis(triphenylphosphine)-nickel(II) dibromide in 2 ml of acetonitrile was heated at 60° C. under argon for 5 minutes. 356 mg (1.00 mmol) of tert-butyl (3RS,4RS)-4-(4-bromophenyl)-3-hydroxy-piperidine-1-carboxylate in solid form were added thereto. The green suspension was stirred at 60° C. under argon for 20 hours. The resulting dark brown suspension was filtered over Speedex and the insoluble material was washed with methylene chloride. The filtrate was partitioned between methylene chloride and 5% sodium hydrogen carbonate solution, the organic phase was dried over magnesium sulfate and finally the solvent was removed under reduced pressure. The crude product (416 mg) was purified by chromatography on silica gel with a 1:1 mixture of ethyl acetate and hexane as the eluent. There were obtained 168 mg (56% of theory) of tert-butyl (3RS,4RS)-4-(4-cyano-phenyl)-3-hydroxy-piperidine-1-carboxylate as a colorless solid; MS: 302 (M)⁺.

(b) In analogy to the procedure described in Example 1(g), by alkylating tert-butyl (3RS,4RS)-4-(4-cyano-phenyl)-3-hydroxy-piperidine-1-carboxylate with 2-bromomethylnaphthalene there was obtained tert-butyl (3RS,4RS)-4-(4-cyano-phenyl)-3-(naphthalen-2-ylmethoxy)-piperidine-1-carboxylate as a light yellow resin; MS: 443 (M+H)⁺.

(c) A solution of 133 mg (0.301 mmol) of tert-butyl (3RS,4RS)-4-(4-cyano-phenyl)-3-(naphthalen-2-ylmethoxy)-piperidine-1-carboxylate in 0.5 ml of tetrahydrofuran was treated with 1.5 ml (1.5 mmol) of a 1M borane-tetrahydrofuran complex solution in tetrahydrofuran and the mixture was heated to reflux under argon for 6 hours. The reaction mixture was partitioned between methylene chloride and water, the organic phase was dried over magnesium sulfate and finally the solvent was removed under reduced pressure. The crude product (163 mg) was purified by chromatography on silica gel with a 14:1:0.1 mixture of methylene chloride, methanol and a 25% ammonia solution as the eluent. There were obtained 106 mg (79% of theory) of tert-butyl (3RS,4RS)-4-(4-aminomethyl-phenyl)-3-(naphthalen-2-ylmethoxy)-piperidine-1-carboxylate as a colorless resin; MS: 447 (M+H)⁺.

(d) A solution of 47 mg (0.105 mmol) of tert-butyl (3RS,4RS)-4-(4-aminomethyl-phenyl)-3-(naphthalen-2-ylmethoxy)-piperidine-1-carboxylate in 2 ml of methylene chloride was treated with 18 .mu.1 (12.7 mg, 0.126 mmol, 1.2 eq.) of triethylamine and cooled to 0° C. 15 μl (20.4 mg, 0.116 mmol, 1.1 eq.) of benzenesulfonyl chloride were added dropwise, the mixture was warmed to room temperature and stirred for 1 hour. The reaction mixture was partitioned between methylene chloride and 5% sodium hydrogen carbonate solution, the organic phase was dried over magnesium sulfate and finally the solvent was removed under reduced pressure. There were obtained 47 mg of crude tert-butyl (3RS,4RS)-4-[4-(phenylsulfonyl-amino-methyl)-phenyl]-3-(naphthalen-2-ylmethoxy)-piperidine-1-carboxylate, which was used in the following step without further purification and characterization.

(e) In an analogous manner to the procedure described under (d), from tert-butyl (3RS,4RS)-4-(4-aminomethyl-phenyl)-3-(naphthalen-2-ylmethoxy)-piperidine-1-carboxylate by acylation with p-anisoyl chloride there was obtained crude tert-butyl (3RS,4RS)-4-[4-(4-methoxy-benzoylamino)-methyl]-phenyl-3-(naphthalen-2-ylmethoxy)-piperidine-1-carboxylate, which was used in the following step without further purification and characterization.

(f) In an analogous manner to the procedure described under (d), from tert-butyl (3RS,4RS)-4-(4-aminomethyl-phenyl)-3-(naphthalen-2-ylmethoxy)-piperidine-1-carboxylate by acylation with phenylacetyl chloride there was obtained crude tert-butyl (3RS,4RS)-3-(naphthalen-2-ylmethoxy)-4-[4-(phenylacetylamino-methyl)-phenyl]-piperidine-1-carboxylate, which was used in the following step without further purification and characterization.

(g) In an analogous manner to that described in Example 1(g), by alkylating tert-butyl (3RS,4RS)-4-(4-cyano-phenyl)-3-hydroxy-piperidine-1-carboxylate with 2-chloromethyl-4-(β-trimethylsilyl-ethoxymethoxy)-naphthalene there was obtained tert-butyl (3RS,4RS)-4-(4-cyano-phenyl)-3-[4-(2-trimethylsilyl-ethoxy-methoxy)-naphthalen-2-ylmethoxy]-piperidine-1-carboxylate as a light yellow oil; MS: 530 [M-(C₂H₄+CH₂O)]⁺.

(h) In analogy to the procedure described under (c), by reducing the nitrile group in tert-butyl (3RS,4RS)-4-(4-cyano-phenyl)-3-[4-(2-trimethylsilyl-ethoxymethoxy)-naphthalen-2-ylmethoxy]-piperidine-1-carboxylate there was obtained tert-butyl (3RS,4RS)-4-(4-aminomethyl-phenyl)-3-[4-(2-trimethylsilyl-ethoxymethoxy)-naphthalen-2-ylmethoxy]-piperidine-1-carboxylate as a light yellow oil; MS: 593 (M+H)⁺.

(i) In analogy to the procedure described under (d), from tert-butyl (3RS,4RS)-4-(4-aminomethyl-phenyl)-3-[4-(2-trimethylsilyl-ethoxymethoxy)-naphthalen-2-ylmethoxy]-piperidine-1-carboxylate by acylation with benzoyl chloride there was obtained crude tert-butyl (3RS,4RS)-4-[4-(benzoylamino-methyl)-phenyl]-3-[4-(2-trimethylsilyl-ethoxymethoxy)-naphthalen-2-ylmethoxy]-piperidine-1-carboxylate, which was used in the following step without further purification and characterization.

### Example 21

The following compounds were obtained in an analogous manner to that described in Example 1(h) by cleavage of the BOC group using a solution of hydrogen chloride in methanol:

1)—(3RS,4RS)-4-(4-Chlorophenyl)-4-hydroxy-3-(4-methoxy-benzyloxy)-piperidine as a colorless solid, MS: 348 (M+H)⁺, from tert-butyl (3RS,4RS)-4-(4-chlorophenyl)-4-hydroxy-3-(4-methoxy-benzyloxy)-piperidine-1-carboxylate;

2)—(3RS,4RS)-4-(4-chlorophenyl)-4-hydroxy-3-(naphthalen-2-ylmethoxy)-piperidine as a colorless solid, MS: 226, 228 [M-(C₁₁H₉)]⁺, from tert-butyl (3RS,4RS)-4-(4-chlorophenyl)-4-hydroxy-3-(naphthalen-2-ylmethoxy)-piperidine-1-carboxylate.

The compounds used as the starting materials were prepared as follows:

(a) A solution of 3.0 g (15.5 mmol) of 4-(4-chlorophenyl)-1,2,3,6-tetrahydro-pyridine in 20 ml of absolute dimethylformamide was treated with 2.37 ml (1.72 g, 17.0 mmol) of triethylamine and cooled to 0° C. A solution of 3.7 g (17.0 mmol) of di-tert-butyl dicarbonate in 8 ml of absolute dimethylformamide was added dropwise at 0° C. The mixture was warmed to room temperature and stirred for 20 hours. The solvent was distilled off at 50-55° C. at 0.1 mm Hg. Subsequently, the residue obtained was partitioned between methylene chloride and water, the organic phase was dried over magnesium sulfate and finally the solvent was removed under reduced pressure. The crude product (5.0 g) was purified by chromatography on silica gel with a 95:5 mixture of methylene chloride and ethyl acetate as the eluent. There were obtained 4.5 g (99% of theory) of tert-butyl 4-(4-chlorophenyl)-1,2,3,6-tetrahydro-pyridine-1-carboxylate as a colorless oil; MS: 236, 238 [M-(C₄H₉)]⁺.

(b) A solution of 2.5 g (8.5 mmol) of tert-butyl 4-(4-chlorophenyl)-1,2,3,6-tetrahydro-pyridine-1-carboxylate in 20 ml of acetone was treated with 0.425 ml (0.0085 mmol, 0.01 eq.) of osmium tetroxide solution (0.02M in tert-butanol) and 8.6 ml of hydrogen peroxide solution (30% in water) and the mixture was stirred at room temperature for 18 hours. The mixture was partitioned between ethyl acetate and water, the organic phase was washed with 10% sodium bisulfite solution and water, dried over magnesium sulfate and finally the solvent was removed under reduced pressure. The crude product (2.2 g) was purified by chromatography on silica gel with a 3:1 mixture of methylene chloride and ethyl acetate as the eluent. There were obtained 544 mg (20% of theory) of tert-butyl (3RS,4RS)-4-(4-chlorophenyl)-3,4-dihydroxy-piperidine-1-carboxylate as a colorless resin; MS: 270, 272 [M-(C₄H₉)]⁺. (c) A solution of 128 mg (0.392 mmol) of tert-butyl (3RS,4RS)-4-(4-chlorophenyl)-3,4-dihydroxy-piperidine-1-carboxylate in 1.5 ml of dimethyl sulfoxide was added dropwise to a suspension of 16 mg (0.4 mmol) of sodium hydride (60% dispersion in refined oil) in 3 ml of dimethyl sulfoxide. After 15 minutes a solution of 61 mg (0.39 mmol) of p-methoxybenzyl chloride in 1 ml of dimethyl sulfoxide was added dropwise at room temperature within 10 minutes and the mixture was stirred for 18 hours. Subsequently, the reaction mixture was partitioned between ethyl acetate and water, the organic phase was dried over magnesium sulfate and finally the solvent was removed under reduced pressure. The crude product (200 mg) was purified by chromatography on silica gel with a 3:1 mixture of hexane and ethyl acetate as the eluent. There were obtained (in addition to starting material and bisalkylated product) 63 mg (38% of theory) of tert-butyl (3RS,4RS)-4-(4-chlorophenyl)-4-hydroxy-3-(4-methoxy-benzyloxy)-piperidine-1-carboxylate as a colorless oil; MS: 448, 450 [M+H]⁺.

(d) In an analogous manner to the procedure described under (c), by alkylating tert-butyl (3RS,4RS)-4-(4-chlorophenyl)-3,4-dihydroxy-piperidine-1-carboxylate with 2-bromomethylnaphthalene there was obtained tert-butyl (3RS,4RS)-4-(4-chlorophenyl)-4-hydroxy-3-(naphthalen-2-ylmethoxy)-piperidine-1-carboxylate as a light yellow oil; MS: 411, 413 [M-(C₄H₈)]⁺.

### Example 22

(a) 400 ml of a 1.2M solution of n-BuLi in hexane were added dropwise within 45 minutes to a solution, cooled to −70° C., of 112.5 g (0.48 mol) of 1,4-dibromobenzene in 1200 ml of diethyl ether in such a manner that the temperature did not rise above −60° C. After completion of the addition the mixture was stirred at −70° C. for 2.5 hours. Thereafter, a solution of 90.84 g (0.48 mol) of 1-benzyl-4-piperidone in 300 ml of ether was added dropwise at −70 to −65° C. during one hour. After the dropwise addition the mixture was stirred at −70° C. for 2 hours. For the working-up, the cold reaction mixture was poured into 1200 ml of a 15% ammonium chloride solution, the mixture was transferred to a separating funnel and the organic phase was separated. The aqueous phase was extracted twice with ether and subsequently the combined organic phases were extracted twice with water and saturated sodium chloride solution. Then, the organic phase was dried over magnesium sulfate evaporated under reduced pressure, with the crude product separating as a yellowish solid. For purification, this was dissolved in hot methylene chloride, the solution was treated with hexane until turbidity began and cooled to room temperature while stirring. The resulting precipitate was filtered off under suction and dried. There were obtained 121.65 g (73% of theory) of 1-benzyl-(4-bromo-phenyl)-piperidin-4-ol as a yellowish solid; m.p.: 106° C., MS: 346, 348 (M+H)⁺.

(b) A mixture of 121.6 g (0.35 mol) of 1-benzyl-4-(4-bromo-phenyl)-piperidin-4-ol and 121.6 g of p-toluenesulfonic acid monohydrate (0.64 mol) in 1200 ml of toluene was heated to reflux on a water separator for 4.5 hours. Subsequently, the reaction mixture was cooled to room temperature and adjusted to pH 10 with 3N sodium hydroxide solution. Thereafter, the mixture was extracted firstly with 200 ml and then with 500 ml of methylene chloride. The combined organic phases were washed twice with 100 ml of water each time, dried over magnesium sulfate and then evaporated under reduced pressure. There were obtained 109.1 g (99% of theory) of 1-benzyl-4-(4-bromo-phenyl)-1,2,3,6-tetrahydro-pyridine as a yellowish solid; MS: 328, 330 (M+H)⁺.

(c) 16.9 g of sodium borohydride were added to a solution of 51.1 g (0.156 mol) of 1-benzyl-4-(4-bromo-phenyl)-1,2,3,6-tetrahydro-pyridine in 350 ml of dimethoxyethane and the reaction mixture was stirred at room temperature for 15 minutes. Thereafter, 95.2 ml of boron trifluoride etherate were added dropwise at 25-30° C. during 30 minutes, the reaction mixture was subsequently stirred at room temperature for 2 hours. Thereafter, firstly a solution of 98.4 g of potassium hydroxide in 530 ml of water was slowly added dropwise and thereafter within 15 minutes 80.7 ml of a 30% hydrogen peroxide solution were added. Subsequently, the mixture was boiled under reflux for 2 hours. For the working-up, the cooled reaction mixture was filtered over Dicalit and this was rinsed with methylene chloride. The solution obtained was treated with 700 ml of methylene chloride, the organic phase was separated and then the aqueous phase was back-extracted with 300 ml of methylene chloride. The combined organic phases were washed twice with 200 ml of water each time, dried over magnesium sulfate and evaporated under reduced pressure. Crystallization of the crude product from acetone yielded 31 g (57% of theory) of (3RS,4RS)-1-benzyl-4-(4-bromo-phenyl)-piperidin-3-ol as colorless crystals; m.p.: 125-129° C.

(d) A Schlenk tube was charged under argon with 74.9 mg (0.29 mmol) of PdCl₂ (CH₃ CN)₂, 168.0 mg (0.303 mmol) of 1,1′-bis(diphenylphosphino)ferrocene and 10 ml of methanol (distilled under argon) and the reaction mixture was stirred at room temperature for 30 minutes. The red-brown suspension was transferred under argon into a 185 ml steel autoclave fitted with a glass insert. Thereafter, 10.0 g (29 mmol) of (3RS,4RS)-1-benzyl-4-(4-bromo-phenyl)-piperidin-3-ol (pre-treated with active charcoal), 60 ml of methanol and 6 ml (43 mmol) of triethylamine (sic) were added. The autoclave was sealed, pressurized to 15 bar with carbon monoxide and the reaction mixture was stirred at 110° for 20 hours under constant pressure. After cooling the autoclave and releasing the gases the orange colored reaction mixture was evaporated under reduced pressure. The solid, orange colored residue was dissolved in 20 ml of methylene chloride, the solution was washed twice with 100 ml of 5% sodium carbonate solution each time and, respectively, with 100 ml of water and then evaporated under reduced pressure. For purification, the yellow-brown solid residue was chromatographed on silica gel using a 1:1 mixture of ethyl acetate and hexane as the eluent. There were obtained 7.74 g (82% of theory) of methyl (3RS,4RS)-4-(1-benzyl-3-hydroxy-piperidin-4-yl)-benzoate as a white solid; m.p.: 103-104° C.; MS: 326 (M+H)⁺.

(e) A solution of 5.0 g (15.3 mmol) of methyl (3RS,4RS)-4-(1-benzyl-3-hydroxy-piperidin-4-yl)-benzoate in 50 ml of tetra-hydrofuran was treated at room temperature with 720 mg (32.9 mmol) of lithium borohydride. Subsequently, the reaction mixture was heated to 60° C. for 15 hours. For the working-up, the reaction mixture was treated with 20 ml of water while cooling with ice and thereafter extracted twice with 50 ml of ethyl acetate each time. The organic phases were combined, dried over sodium sulfate and evaporated under reduced pressure. The crude (3RS,4RS)-1-benzyl-4-(4-hydroxymethyl-phenyl)-piperidine-3-ol obtained (R_{f}: 0.23, methylene chloride:methanol:ammonia=95:5:0.1) was used in the following step without further purification.

(f) A solution of 2.0 g (6.72 mmol) of (3RS,4RS)-1-benzyl-4-(4-hydroxymethyl-phenyl)-piperidin-3-ol in 100 ml of ethanol was hydrogenated at room temperature and 3 bar for 4 hours in the presence of 1.0 g of palladium oxide/charcoal (20%). For the working-up, the catalyst was filtered off under suction over Dicalit and the residue was washed twice with 50 ml of ethanol each time. The ethanol solution was evaporated under reduced pressure and the residue was chromatographed on silica gel using a 65:10:1 mixture of methylene chloride, methanol and ammonia as the eluent. There were obtained 1.1 g (79% of theory) of (3RS,4RS)-4-(4-hydroxymethyl-phenyl)-piperidin-3-ol as a colorless solid; MS: 207 (M)⁺.

(g) A solution of 11.10 g (5.31 mmol) of (3RS,4RS)-4-(4-hydroxymethyl-phenyl)-piperidin-3-ol in 20 ml of dimethylformamide was treated at 0° C. with 0.59 g (5.82 mmol) of triethylamine and 1.22 g (5.57 mmol) of di-tert-butyl dicarbonate and the mixture was stirred at room temperature for 15 hours. Subsequently, the dimethylformamide was distilled off in an oil pump vacuum and, for purification, the residue was chromatographed on silica gel using a 98:2 mixture of methylene chloride and methanol as the eluent. There were obtained 1.51 g (92% of theory) of tert-butyl (3RS,4RS)-3-hydroxy-4-(4-hydroxymethyl-phenyl)-piperidine-1-carboxylate as a colorless foam; MS: 233 (M-C₄H₁₀O)⁺.

(h) A solution of 1.50 g (5.14 mmol) of tert-butyl (3RS,4RS)-3-hydroxy-4-(4-hydroxymethyl-phenyl)-piperidine-1-carboxylate, 1.73 g (6.16 mmol) of triphenylchloromethane and 674 mg (6.68 mmol) of triethylamine in 20 ml of methylene chloride was stirred at room temperature for 5 hours. For the working-up, the reaction mixture was washed with 10 ml of water and 10 ml of saturated sodium hydrogen carbonate solution, the organic phase was dried over sodium sulfate and evaporated under reduced pressure. The crude product was chromatographed on silica gel using a 95:5 mixture of toluene and ethyl acetate as the eluent. There were obtained 2.08 g (77.5% of theory) of tert-butyl (3RS,4RS)-3-hydroxy-4-(4-trityloxymethyl-phenyl)-piperidine-1-carboxylate as a colorless foam; MS: 567 (M+NH₄)⁺.

(i) 290 mg (6.05 mmol) of sodium hydride (50% dispersion in refined oil) were added to a solution of 2.08 g (3.78 mmol) of tert-butyl (3RS,4RS)-3-hydroxy-4-(4-trityloxymethyl-phenyl)-piperidine-1-carboxylate and 1.0 g (4.54 mmol) of 2-bromomethylnaphthalene in 30 ml of dimethylformamide and the reaction mixture was stirred at room temperature for 2 hours. For the working-up, the reaction mixture was evaporated in an oil pump vacuum, the residue was partitioned between 100 ml of saturated ammonium chloride solution and 100 ml of ethyl acetate and thereafter the separated aqueous phase was extracted twice with 50 ml of ethyl acetate each time. The combined ethyl acetate extracts were dried over sodium sulfate and evaporated under reduced pressure. For purification, the crude product was chromatographed on silica gel using a 4:1 mixture of methylene chloride and hexane as the eluent. There were obtained 2.27 g (87% of theory) of tert-butyl (3RS,4RS)-3-naphthalen-2-ylmethoxy-4-(4-trityloxymethyl-phenyl)-piperidine-1-carboxylate as a colorless solid; MS: 707 (M+NH₄)⁺.

(j) A solution of 1.07 g (1.48 mmol) of tert-butyl (3RS,4RS)-3-naphthalen-2-ylmethoxy-4-(4-trityloxymethyl-phenyl)-piperidine-1-carboxylate in 15 ml of methylene chloride was treated at room temperature with 2 ml of 2N hydrogen chloride in methanol and the mixture was stirred at room temperature for 15 minutes. Subsequently, the solution was poured into 30 ml of saturated sodium carbonate solution and this was extracted twice with 50 ml of ethyl acetate each time. The combined organic phases were dried over sodium sulfate and evaporated under reduced pressure. For purification, the crude product was chromatographed on silica gel using a 99:1 mixture of methylene chloride and methanol as the eluent. There were obtained 580 mg (82% of theory) of tert-butyl (3RS,4RS)-4-(4-hydroxymethyl-phenyl)-3-(naphthalen-2-ylmethoxy)-piperidine-1-carboxylate as a colorless oil; MS: 447 (M)⁺.

(k) A solution of 45 mg (0.1 mmol) of tert-butyl (3RS,4RS)-4-(4-hydroxymethyl-phenyl)-3-(naphthalen-2-ylmethoxy)-piperidine-1-carboxylate, 12 mg (0.12 mmol) of triethylamine and 12 mg (0.1 mmol) of pivaloyl chloride in 5 ml of methylene chloride was stirred at room temperature for 15 hours. For the working-up, the reaction solution was diluted with 10 ml of methylene chloride, then washed with 5 ml of water, dried over sodium sulfate and evaporated under reduced pressure. The residue was chromatographed on silica gel using a 4:1 mixture of hexane, and ethyl acetate as the eluent. There were obtained 39 mg (73% of theory) of tert-butyl (3RS,4RS)-4-[4-(2,2-dimethyl-propionyloxymethyl)-phenyl]-3-(naphthalen-2-ylmethoxy)-piperidine-1-carboxylate as a colorless oil; MS: 549 (M+NH₄)⁺.

(l) A solution of 35 mg (0.07 mmol) of tert-butyl (3RS,4RS)-4-[4-(2,2-dimethyl-propionyloxymethyl)-phenyl]-3-(naphthalen-2-ylmethoxy)-piperidine-1-carboxylate in 2 ml of 2M hydrogen chloride in methanol was stirred at room temperature for 4.5 hours. Subsequently, the reaction solution was evaporated under reduced pressure. The residue was taken up in diethyl ether, with a part (15 mg, 49% of theory) of the (3RS,4RS)-4-(3-naphthalen-2-ylmethoxy-piperidin-4-yl)-benzyl 2,2-dimethyl-propionate hydrochloride being obtained in the form of white crystals; MS: 432 (M+H)⁺.

### Example 23

A solution of 1.10 g (1.59 mmol) of tert-butyl (3RS,4RS)-3-naphthalen-2-ylmethoxy-4-(4-trityloxymethyl-phenyl)-piperidine-1-carboxylate in 50 ml of methanol was treated at room temperature with 30 ml of a 2M solution of hydrogen chloride in methanol and the mixture was stirred at room temperature for 4 hours. Subsequently, the solution was evaporated under reduced pressure and the residue was partitioned between 30 ml of saturated sodium carbonate solution and 50 ml of ethyl acetate. The aqueous phase was again extracted with 50 ml of ethyl acetate, thereafter the organic phases were combined, dried over sodium sulfate and evaporated under reduced pressure. For purification, the crude product was chromatographed on silica gel using a 90:10 mixture of methylene chloride and methanol as the eluent. There were obtained 462 mg (83% of theory) of (3RS,4RS)-{4-[3-(naphthalen-2-ylmethoxy)-piperidin-4-yl]-phenyl}-methanol as a colorless solid; MS: 348 (M+H)⁺.

### Example 24

The following compounds were obtained in an analogous manner to that in Example 22 (l) by cleavage of the BOC group using acid

1)—(3RS,4RS)-4-[3-(Naphthalen-2-ylmethoxy)-piperidin-4-yl]-benzyl benzoate as a colorless foam, MS: 452 (M+H)⁺, from tert-butyl (3RS,4RS)-4-(4-benzoyloxymethyl-phenyl)-3-(naphthalen-2-ylmethoxy)-piperidine-1-carboxylate;

2)—(3RS,4RS)-4-(3-naphthalen-2-ylmethoxy-piperidin-4-yl)-benzyl 3-methoxy-benzoate hydrochloride as a colorless solid, MS: 482 (M+H)⁺, from tert-butyl (3RS,4RS)-4-[4-(3-methoxy-benzoyloxymethyl)-phenyl]-3-naphthalen-2-ylmethoxy-piperidine-1-carboxylate;

3)—(3RS,4SR)-4-(3-naphthalen-2-ylmethoxy-piperidin-4-yl)-benzyl 3,5-dimethoxy-benzoate hydrochloride as a colorless solid, MS: 512 (M+H)⁺, from tert-butyl (3RS,4RS)-4-[4-(3,5-dimethoxy-benzoyloxymethyl)-phenyl]-3-(naphthalen-2-yl methoxy)-piperidine-1-carboxylate;

4)—(3RS,4RS)-4-[3-(naphthalen-2-ylmethoxy)-piperidin-4-yl]-benzyl cyclohexanecarboxylate trifluoroacetate as a colorless solid, MS: 458 (M+H)⁺, from tert-butyl (3RS,4RS)-4-(4-cyclohexanecarbonyloxymethyl-phenyl)-3-(naphthalen-2-ylmethoxy)-piperidine-1-carboxylate using trifluoroacetic acid in methylene chloride;

5)—(4RS,3RS)-4-(3-naphthalen-2-ylmethoxy-piperidin-4-yl)-benzyl 2-chloro-benzoate trifluoroacetate as a colorless solid, MS: 486 (M+H)⁺, from tert-butyl (3RS,4RS)-[4-(2-chloro-benzoyloxymethyl)-phenyl]-3-naphthalen-2-ylmethoxy-piperidine-1-carboxylate using trifluoroacetic acid in methylene chloride;

6)—carbonic acid methyl ester (3RS,4RS)-4-(naphthalen-2-ylmethoxy-piperidin-4-yl)-benzyl ester hydrochloride as a colorless oil, MS: 406 (M+H)⁺, from tert-butyl (3RS,4RS)-4-(4-methoxycarbonyloxymethyl-phenyl)-3-(naphthalen-2-ylmethoxy)-piperidine-1-carboxylate;

7)—(3RS,4RS)-4-[3-(naphthalen-2-ylmethoxy)-piperidin-4-yl]-benzyl pyridine-4-carboxylate hydrochloride as a yellow oil, MS: 453 (M+H)⁺, from (3RS,4RS)-4-(1-tert-butoxycarbonyl-3-naphthalen-2-ylmethoxy-piperidin-4-yl)-benzyl pyridine-4-carboxylate;

8)—(3RS,4RS)-4-(3-naphthalen-2-ylmethoxy-piperidin-4-yl)-benzyl pyrazine-2-carboxylate hydrochloride as a colorless solid, MS: 454 (M+H)⁺, from (3RS,4RS)-4-[1-tert-butoxycarbonyl-3-(naphthalen-2-ylmethoxy)-piperidin-4-yl]-benzyl pyrazine-2-carboxylate;

9)—(3RS,4RS)-4-[3-(1-methoxy-naphthalen-2-ylmethoxy)-piperidin-4-yl]-benzyl 2-chloro-benzoate trifluoroacetate as a colorless solid, MS: 516 (M+H)⁺, from tert-butyl (3RS,4RS)-4-[4-(2-chloro-benzoyloxymethyl)-phenyl]-3-(1-methoxy-naphthalen-2-ylmethoxy)-piperidine-1-carboxylate using trifluoroacetic acid in methylene chloride;

10)—(3RS,4RS)-4-(3-naphthalen-2-ylmethoxy-piperidin-4-yl)-benzyl 4-hydroxy-benzoate hydrochloride as a yellowish solid, MS: 468 (M+H)⁺, from tert-butyl (3RS,4RS)-4-[4-(4-hydroxy-benzoyl-oxymethyl)-phenyl]-3-(naphthalen-2-ylmethoxy)-piperidine-1-carboxylate using trifluoroacetic acid in methylene chloride and simultaneous cleavage of the acetal group;

11)—(3RS,4RS)-4-[3-(naphthalen-2-ylmethoxy)-piperidin-4-yl)-benzyl pyridin-2-yl-carbamate hydrochloride as a colorless foam, R.sub.f: 0.15 (methylene chloride:methanol:ammonia=90:10:0.1), from tert-butyl (3RS,4RS)-3-(naphthalen-2-ylmethoxy)-4-[4-(pyridin-2-ylcarbamoyloxymethyl)-phenyl]-piperidine-1-carboxylate;

12)—(3RS,4RS)-4-[4-(3-methoxy-propenyl)-phenyl]-3-(naphthalen-2-ylmethoxy)-piperidine as a yellow oil, MS: 388 (M+H)⁺, from tert-butyl (3RS,4RS)-4-[4-(3-hydroxy-propenyl)-phenyl]-3-(naphthalen-2-ylmethoxy)-piperidine-1-carboxylate;

13)—(3RS,4RS)-4-[4-(3-benzoyloxy-propenyl)-phenyl]-3-(naphthalen-2-ylmethoxy)-piperidine as a yellow oil, MS: 478 (M+H)⁺, from tert-butyl (3RS,4RS)-4-[4-(3-benzoyloxy-propenyl)-phenyl]-3-(naphthalen-2-ylmethoxy)-piperidine-1-carboxylate;

14)—3-[4-[3-(naphthalen-2-ylmethoxy)-piperidin-4-yl]-phenyl]-propyl (3RS,4RS)-2-chloro-benzoate as a yellowish oil, MS: 514, 516 (M+H)⁺, from tert-butyl (3RS,4RS)-4-[4-[3-(2-chloro-benzoyloxy)-propyl)-phenyl]-3-(naphthalen-2-yl methoxy)-piperidine-1-carboxylate.

The starting materials were obtained as follows analogously to the procedure described in Example 22(k):

(a) tert-Butyl (3RS,4RS)-4-(4-benzoyloxymethyl-phenyl)-3-(naphthalen-2-ylmethoxy)-piperidine-1-carboxylate as a colorless oil, MS: 552 (M+H)⁺, from tert-butyl (3RS,4RS)-4-(4-hydroxymethyl-phenyl)-3-(naphthalen-2-ylmethoxy)-piperidine-1-carboxylate and benzoyl chloride.

(b) tert-Butyl (3RS,4RS)-4-[4-(3-methoxy-benzoyloxymethyl)-phenyl]-3-naphthalen-2-ylmethoxy-piperidine-1-carboxylate as a colorless oil, MS: 582 (M+H)⁺, from tert-butyl (3RS,4RS)-4-(4-hydroxymethyl-phenyl)-3-(naphthalen-2-ylmethoxy)-piperidine-1-carboxylate and 3-methoxybenzoyl chloride.

(c) tert-Butyl (3RS,4RS)-4-[4-(3,5-dimethoxy-benzoyloxy-methyl)-phenyl]-3-(naphthalen-2-ylmethoxy)-piperidine-1-carboxylate as a colaurless oil, MS: 612 (M+H)⁺, from tert-butyl (3RS,4RS)-4-(4-hydroxymethyl-phenyl)-3-(naphthalen-2-yl-methoxy)-piperidine-1-carboxylate and 3,5-dimethoxybenzoyl chloride.

(d) tert-Butyl (3RS,4RS)-4-(4-cyclohexanecarbonyloxymethyl-phenyl)-3-(naphthalen-2-ylmethoxy)-piperidine-1-carboxylate as a colorless oil, MS: 558 (M+H)⁺, from tert-butyl (3RS,4RS)-4-(4-hydroxymethyl-phenyl)-3-(naphthalen-2-ylmethoxy)-piperidine-1-carboxylate and cyclohexanecarbonyl chloride.

(e) tert-Butyl (3RS,4RS)-[4-(2-chloro-benzoyloxymethyl)-phenyl]-3-naphthalen-2-ylmethoxy-piperidine-1-carboxylate as a colorless oil, MS: 586 (M+H)⁺, from tert-butyl (3RS,4RS)-4-(4-hydroxymethyl-phenyl)-3-(naphthalen-2-ylmethoxy)-piperidine-1-carboxylate and 2-chlorobenzoyl chloride.

(f) tert-Butyl (3RS,4RS)-4-(4-methoxycarbonyloxymethyl-phenyl)-3-(naphthalen-2-ylmethoxy)-piperidine-1-carboxylate as a colorless oil from tert-butyl (3RS,4RS)-4-(4-hydroxymethyl-phenyl)-3-(naphthalen-2-ylmethoxy)-piperidine-1-carboxylate and methyl chloroformate.

(g) A solution of 60 mg (0.13 mmol) of tert-butyl (3RS,4RS)-4-(4-hydroxymethyl-phenyl)-3-(naphthalen-2-ylmethoxy)-piperidine-1-carboxylate, 16 mg (0.13 mmol) of isonicotinic acid, 34 mg (0.26 mmol) of ethyldiisopropylamine and 58 mg (0.13 mmol) of benzotriazol-1-yl-oxy-tris-(dimethylamino)-phosphonium hexafluorophosphate (BOP) in 5 ml of acetonitrile was stirred at room temperature for 2 hours. Subsequently, the reaction mixture was evaporated under reduced pressure and the residue, without further working-up, was chromatographed on silica gel using a 3:1 mixture of hexane and ethyl acetate as the eluent. There were obtained 15 mg (21% of theory) of (3RS,4RS)-4-(1-tert-butoxycarbonyl-3-naphthalen-2-ylmethoxy-piperidin-4-yl)-benzylpyridine-4-carboxylate as a colorless solid; MS: 552 (M)⁺.

(h) In an analogous manner to that described under (g) by condensing tert-butyl (3RS,4RS)-4-(4-hydroxymethyl-phenyl)-3-(naphthalen-2-ylmethoxy)-piperidine-1-carboxylate and pyrazinecarboxylic acid using 1,1-carbonyldiimidazole as the condensation agent there was obtained 4-[1-tert-butoxycarbonyl-3-(naphthalen-2-ylmethoxy)-piperidin-4-yl]-benzyl (3RS,4RS)-pyrazine-2-carboxylate; MS: 554 (M+H)⁺.

Processing was carried out as follows in an analogous manner to that described in Example 22 (i)-(k):

(i) Alkylation of methyl (3RS,4RS)-4-(1-benzyl-3-hydroxy-piperidin-4-yl)-benzoate with 2-bromomethyl-1-methoxy-naphthalene gave tert-butyl (3RS,4RS)-3-(1-methoxy-naphthalen-2-ylmethoxy)-4-(4-trityloxymethyl-phenyl)-piperidine-1-carboxylate as a colorless oil; MS: 720 (M+H)⁺.

(j) Cleavage of the trityl group from tert-butyl (3RS,4RS)-3-(1-methoxy-naphthalen-2-ylmethoxy)-4-(4-trityloxymethyl-phenyl)-piperidine-1-carboxylate yielded tert-butyl (3RS,4RS)-4-(4-hydroxymethyl-phenyl)-3-(1-methoxy-naphthalen-2-ylmethoxy)-piperidine-1-carboxylate as a colorless oil; MS: 478 (M+H)⁺.

(k) Acylation of tert-butyl (3RS,4RS)-4-(4-hydroxymethyl-phenyl)-3-(1-methoxy-naphthalen-2-ylmethoxy)-piperidine-1-carboxylate with 2-chlorobenzoyl chloride gave tert-butyl (3RS,4RS)-4-[4-(2-chloro-benzoyloxymethyl)-phenyl]-3-(1-methoxy-naphthalen-2-ylmethoxy)-piperidine-1-carboxylate as a colorless oil; MS: 615 (M+H)⁺.

(l) In an analogous manner to that described under (g) by condensing tert-butyl (3RS,4RS)-4-(4-hydroxyethyl-phenyl)-3-(naphthalen-2-ylmethoxy)-piperidine-1-carboxylate and 4-(2-trimethylsilanyl-ethoxymethoxy)-benzoic acid using N-ethyl-N′-(3-dimethylaminopropyl)-carbodiimide hydrochloride (EDC) as the condensation agent there was obtained tert-butyl (3RS,4RS)-3-(naphthalen-2-ylmethoxy)-4-{4-[4-(2-trimethylsilanyl-ethoxymethoxy)-benzoyloxymethyl]-phenyl}-piperidine-1-carboxylate; MS: 715 (M+NH4)⁺.

The 4-(2-trimethylsilanyl-ethoxymethoxy)-benzoic acid was obtained as a colorless solid in an analogous manner to that described in Example 5 by reaction of methyl 4-hydroxybenzoate with 2-(trimethylsilyl)-ethoxymethyl chloride and subsequent basic saponification of the ester.

(m) A solution of 57 mg (0.13 mmol) of tert-butyl (3RS,4RS)-4-(4-hydroxymethyl-phenyl)-3-(naphthalen-2-ylmethoxy)-piperidine-1-carboxylate, 20 mg (0.14 mmol) of pyridine-2-carbonyl azide [H. Saikachi and T. Kitgawa, Chem. Pharm. Bull. 25 (7), 1651-1657 (1977)] and 3 mg of 4-dimethylaminopyridine in 3 ml of toluene was heated to 90° C. for 3 hours. Subsequently, the toluene was evaporated under reduced pressure, the residue was taken up in 15 ml of methylene chloride and the solution was washed with 5 ml of water. The organic phase was thereafter dried over sodium sulfate and evaporated under reduced pressure. The oily residue was taken up with a mixture of ether and hexane and crystallized. There were obtained 64 mg (88% of theory) of tert-butyl (3RS,4RS)-3-(naphthalen-2-ylmethoxy)-4-[4-(pyridin-2-ylcarbamoyloxy-methyl)-phenyl]-piperidine-1-carboxylate as a colorless solid; MS: 568 (M+H)⁺.

(n) A Schlenk tube was charged under argon with 25.6 mg (0.114 mmol) of Pd(OAc)₂, 69.6 mg (0.Z29 mmol) of tri(o-tolyl)phosphine and 20 ml of DMF (distilled under argon) and the reaction mixture was stirred at room temperature for 15 minutes. A 200 ml sulfonation flask was charged under argon and while stirring with 8.15 g (22.9 mmol) of tert-butyl (3RS,4RS)-4-(4-bromo-phenyl)-3-hydroxy-piperidine-1-carboxylate [Example 25 (c)], 100 ml of DMF, 3.73 ml (34.3 mmol) of ethyl acrylate, 2.25 g (27.5 mmol) of sodium acetate and the yellow catalyst solution. The reaction mixture was stirred at 120° C. for 6 hours. In order to complete the reaction, a solution of 5.1 mg Pd(OAc)₂ and 14.3 mg of tri(o-tolyl)phosphine in 5 ml of DMF was added after 5 hours. The dark reaction mixture was evaporated on a rotary evaporator. The grey solid residue was taken up in ether and the turbid solution was washed three times with water, dried over sodium sulfate and evaporated under reduced pressure. The yellow solid residue was chromatographed on 250 g of silica gel using a 2:1 mixture of hexane and ethyl acetate as the eluent. After crystallization from ethyl acetate there were obtained 6.66 g (77% of theory) of tert-butyl (3RS,4RS)-4-[4-(2-ethoxycarbonyl-vinyl)-phenyl]-3-hydroxy-piperidine-1-carboxylate as colorless crystals; MS: 376 (M+H)⁺.

(o) Alkylation of tert-butyl (3RS,4RS)-4-[4-(2-ethoxycarbonyl-vinyl)-phenyl]-3-hydroxy-piperidine-1-carboxylate with 2-bromomethylnaphthalene analogously to the procedure described in Example 22(i) gave tert-butyl (3RS,4RS)-4-[4-(2-ethoxycarbonyl-vinyl)-phenyl]-3-(naphthalen-2-ylmethoxy)-piperidine-1-carboxylate as a light yellow resin; MS: 516 (M+H)⁺.

(p) 5 ml (5 mmol) of a solution of diisobutylaluminium hydride (DIBAH) (1 M in hexane) were added dropwise at −50° C. to a solution of 698 mg (1.35 mmol) of tert-butyl (3RS,4RS)-4-[4-(2-ethoxy-carbonyl-vinyl)-phenyl]-3-(naphthalen-2-ylmethoxy)-piperidine-1-carboxylate in 15 ml of toluene and the mixture was stirred at −50° C. for 30 minutes. Subsequently, the reaction mixture was treated dropwise at −50° C. with 10 ml of ethanol and warmed to room temperature. For the working-up, the reaction mixture was treated with 20 ml of water and 20 ml of saturated potassium sodium tartrate solution while cooling with ice and thereafter extracted three times with 50 ml of ethyl acetate each time. The organic phases were combined, dried over sodium sulfate and evaporated under reduced pressure. The crude product was chromatographed on silica gel using a 6:4 mixture of hexane and ethyl acetate as the eluent. There were obtained 354 mg (55% of theory) of tert-butyl (3RS,4RS)-4-[4-(3-hydroxy-propenyl)-phenyl]-3-(naphthalen-2-ylmethoxy)-piperidine-1-carboxylate as a colorless solid; MS: 474 (M+H)⁺.

(q) Acylation of tert-butyl (3RS,4RS)-4-[4-(3-hydroxy-propenyl)-phenyl]-3-(naphthalen-2-ylmethoxy)-piperidine-1-carboxylate with benzoyl chloride analogously to the procedure described in Example 22(k) yielded tert-butyl (3RS,4RS)-4-[4-(3-benzoyloxy-propenyl)-phenyl]-3-(naphthalen-2-ylmethoxy)-piperidine-1-carboxylate as a colorless resin; MS: 578 (M+H)⁺.

(r) A solution of 2.0 g (5.35 mmol) of tert-butyl (3RS,4RS)-4-[4-(2-ethoxycarbonyl-vinyl)-phenyl]-3-hydroxy-piperidine-1-carboxylate in 100 ml of ethanol was treated with 200 mg of palladium/charcoal (Type E101R) and hydrogenated at room temperature for 1 hour. Subsequently, the catalyst was filtered off and rinsed with ethanol. The filtrate was evaporated under reduced pressure and the light grey residue (1.97 g) was combined with those from three analogous hydrogenation batches (total 3.08 g). For purification, the crude product was chromatographed on silica gel using a 2:1 mixture of hexane and ethyl acetate as the eluent and thereafter crystallized from ethyl acetate/hexane. There were obtained 2.67 g (87% of theory) of tert-butyl (3RS,4RS)-4-[4-(2-ethoxycarbonyl-ethyl)-phenyl]-3-hydroxy-piperidine-1-carboxylate as colorless crystals; MS: 378 (M+H)⁺.

(s) Analogously to the procedure described in Example 22(i), by alkylating tert-butyl (3RS,4RS)-4-[4-(2-ethoxycarbonyl-ethyl)-phenyl]-3-hydroxy-piperidine-1-carboxylic acid with 2-bromo-methylnaphthalene there was obtained tert-butyl (3RS,4RS)-4-[4-(2-ethoxycarbonyl-ethyl)-phenyl]-3-(naphthalen-2-ylmethoxy)-piperidine-1-carboxylate; MS: 518 (M+H)⁺.

(t) Analogously to the procedure described in Example 22(e), by reducing tert-butyl (3RS,4RS)-4-[4-(2-ethoxycarbonyl-ethyl)-phenyl]-3-(naphtalen-2-ylmethoxy)-piperidine-1-carboxylate using lithium borohydride there was obtained tert-butyl (3RS,4RS)-4-[4-(3-hydroxy-propyl)-phenyl]-3-(naphthalen-2-ylmethoxy)-piperidine-1-carboxylate as a colorless solid; MS: 476 (M+H)⁺.

(u) Acylation of tert-butyl (3RS,4RS)-4-[4-(3-hydroxy-propyl)-phenyl]-3-(naphthalen-2-ylmethoxy)-piperidine-1-carboxylate with 2-chloro-benzoyl chloride analogously to the procedure described in Example 22(k) gave tert-butyl (3RS,4RS)-4-[4-[3-(2-chloro-benzoyloxy)-propyl]-phenyl]-3-(naphthalen-2-yl methoxy)-piperidine-1-carboxylate as a colorless, amorphous solid; MS: 614.6, 616 (M+H)⁺.

### Example 25

(a) A mixture of 1.0 g (2.9 mmol) of (3RS,4RS)-1-benzyl-4-(4-bromo-phenyl)-piperidin-3-ol [Example 22(c)], 1.36 ml (10.2 mmol) of 2,2,2-trichloroethyl chloroformate and 0.90 g (12.6 mmol) of lithium carbonate in 20 ml of toluene was heated to 105° C. for 18 hours. For the working-up, the cooled reaction mixture was poured into 200 ml of ice-water and subsequently extracted three times with 25 ml of ethyl acetate each time. The combined organic phases were dried over sodium sulfate and evaporated under reduced pressure. For purification, the crude material obtained was chromatographed on silica gel using a 2:3 mixture of methylene chloride and hexane as the eluent. There were obtained 1.3 g (76% of theory) of 2,2,2-trichloroethyl (3RS,4RS)-4-(4-bromo-phenyl)-3-(2,2,2-trichloro-ethoxycarbonyloxy)-piperidine-1-carboxylate as a colorless solid; R_{f}: 0.17 (methylene chloride:hexane=1:1), MS: 622, 624, 626 (M+NH₄)⁺.

(b) A mixture of 1.3 g (2.1 mmol) of 2,2,2-trichloroethyl (3RS,4RS)-4-(4-bromo-phenyl)-3-(2,2,2-trichloro-ethoxy-carbonyloxy)-piperidine-1-carboxylate 1.54 g of activated zinc in 20 ml of glacial acetic acid was stirred at room temperature for 5 hours. For the working-up, the zinc was filtered off, the residue was rinsed with glacial acetic acid and the solution was subsequently evaporated to dryness under reduced pressure. The residue was partitioned between 20 ml of saturated sodium carbonate solution and 30 ml of ethyl acetate, thereafter the separated aqueous phase was extracted twice with 30 ml of ethyl acetate each time. The combined organic phases were dried over sodium sulfate and evaporated under reduced pressure. Crystallization of the residue using a 1:2 mixture of ethyl acetate and hexane yielded 250 mg (46% of theory) of (3RS,4RS)-4-(4-bromo-phenyl)-piperidin-3-ol as a colorless solid; MS: 255, 257 (M)⁺. The mother liquor was purified by chromatography on silica gel using a 4:1 mixture of methylene chloride and methanol as the eluent. A further 101 mg (18% of theory) were obtained.

(c) A solution of 351 mg (1.37 mmol) of (3RS,4RS)-4-(4-bromo-phenyl)-piperidin-3-ol in 12 ml of dimethylformamide was treated with 139 mg (1.37 mmol) of triethylamine and 300 mg (1.37 mg) of di-tert-butyl dicarbonate and the mixture was stirred at room temperature for 15 hours. Subsequently, the dimethylformamide was distilled off in an oil pump vacuum and the residue was crystallized from a 1:1 mixture of ether and hexane. There were obtained 318 mg (65% of theory) of tert-butyl (3RS,4RS)-4-(4-bromophenyl)-3-hydroxy-piperidine-1-carboxylate as a colorless solid; MS: 299, 301 (M-C₄H₈)⁺. Chromatography of the mother liquor on silica gel using a 98:2 mixture of methylene chloride and methanol as the eluent yielded a further 96 mg (19% of theory) of the product.

(d) In an analogous manner to that described in Example 22(d), by carbonylating tert-butyl (3RS,4RS)-4-(4-bromo-phenyl)-3-hydroxy-piperidine-1-carboxylate using PdCl₂ (CH₃CN)₂ and 1,3-bis(diphenylphosphino)-propane as the catalyst in the presence of triethylamine under 10 bar of carbon monoxide at 100° C. for 40 hours there was obtained tert-butyl (3RS,4RS)-3-hydroxy-4-(4-methoxycarbonylphenyl)-piperidine-1-carboxylate as white crystals; m.p.: 145.5-146° C., MS: 279 (M-C₄H₈)⁺.

(e) In an analogous manner to that described in Example 22(i), by alkylating tert-butyl (3RS,4RS)-3-hydroxy-4-(4-methoxycarbonyl-phenyl)-piperidine-1-carboxylate with 2-bromomethylnaphthalene there was obtained tert-butyl (3RS,4RS)-4-(4-methoxycarbonyl-phenyl)-3-naphthalen-2-ylmethoxy-piperidine-1-carboxylate as a colorless oil; MS: 476 (M+H)⁺.

(f) In an analogous manner to that described in Example 22(l), after cleavage of the BOC group from tert-butyl (3RS,4RS)-4-(4-methoxycarbonyl-phenyl)-3-naphthalen-2-ylmethoxy-piperidine-1-carboxylate there was obtained methyl (3RS,4RS)-4-(3-naphthalen-2-ylmethoxy-piperidin-4-yl)-benzoate hydrochloride as a colorless solid; MS: 344 (M-OCH₃)⁺.

### Example 26

The following compounds were obtained in an analogous manner to that described in Example 22(l) by cleavage of the BOC group:

1)—(3RS,4RS)-[3-[3-(4-Benzyloxy-naphthalen-2-ylmethoxy)-piperidin-4-yl]-phenyl}-methanol as a colorless amorphous powder, MS: 454 (M+H)⁺, from tert-butyl (3RS,4RS)-3-(4-benzyloxy-naphthalen-2-ylmethoxy)-4-(3-hydroxymethyl-phenyl)-piperidine-1-carboxylate;

2)—(3RS,4RS)-3-(3-naphthalen-2-ylmethoxy-piperidin-4-yl)-benzyl benzoate hydrochloride as a colorless solid, MS: 452 (M+H)⁺, from tert-butyl (3RS,4RS)-4-(3-benzoyloxymethyl-phenyl)-3-naphthalen-2-ylmethoxy-piperidine-1-carboxylate.

The BOC derivatives used as the starting materials were obtained as follows:

(a) In an analogous manner to that described in Example 22(a)-(d), starting from 3-bromophenyllithium and 1-benzyl-4-piperidone there was obtained methyl (3RS,4RS)-3-(1-benzyl-3-hydroxy-piperidin-4-yl)-benzoate as a light yellow resin; MS: 325 (M)⁺.

(b) The benzyl group was cleaved off from methyl (3RS,4RS)-3-(1-benzyl-3-hydroxy-piperidin-4-yl)-benzoate by catalytic hydrogenation in an analogous manner to that described in Example 2(e). The methyl (3RS,4RS)-3-(3-hydroxy-piperidine-4-yl)-benzoate was converted, without further purification and characterization, with di-tert-butyl dicarbonate analogously to Example 22(g) into tert-butyl (3RS,4RS)-3-hydroxy-4-(3-methoxycarbonyl-phenyl)-piperidine-1-carboxylate, MS: 304 (M-OCH₃)⁺.

(c) In an analogous manner to that described in Example 22(i), by alkylating tert-butyl (3RS,4RS)-3-hydroxy-4-(3-methoxycarbonyl-phenyl)-piperidine-1-carboxylate with 1-benzyloxy-3-chloromethylnaphthalene there was obtained tert-butyl (3RS,4RS)-3-(4-benzyloxy-naphthalen-2-ylmethoxy)-4-(3-methoxycarbonyl-phenyl)-piperidine-1-carboxylate as a pale yellow amorphous powder; MS: 582 (M+H)⁺.

(d) In an analogous manner to that described in Example 22(e), by reducing tert-butyl (3RS,4RS)-3-(4-benzyloxy-naphthalen-2-ylmethoxy)-4-(3-methoxycarbonyl-phenyl)-piperidine-1-carboxylate with lithium borohydride there was obtained tert-butyl (3RS,4RS)-3-(4-benzyloxy-naphthalen-2-ylmethoxy)-4-(3-hydroxymethyl-phenyl)-piperidine-1-carboxylate as a colorless solid; MS: 554 (M+H)⁺.

(e) In an analogous manner to that described in Example 22(i), by alkylating tert-butyl (3RS,4RS)-3-hydroxy-4-(3-methoxycarbonyl-phenyl)-piperidine-1-carboxylate with 2-bromomethylnaphthalene there was obtained tert-butyl (3RS,4RS)-4-(3-methoxycarbonyl-phenyl)-3-naphthalen-2-ylmethoxy-piperidine-1-carboxylate as a colorless oil; MS: 493 (M+NH₄)⁺.

(f) In an analogous manner to that described in Example 22(e), by reducing tert-butyl (3RS,4RS)-4-(3-methoxycarbonyl-phenyl)-3-naphthalen-2-ylmethoxy-piperidine-1-carboxylate with lithium borohydride there was obtained tert-butyl 4-(3-hydroxymethyl-phenyl)-3-(naphthalen-2-ylmethoxy)-piperidine-1-carboxylate as a colorless oil; MS: 448 (M+H)⁺.

(g) In an analogous manner to that described in Example 22(k), by acylating tert-butyl (3RS,4RS)-4-(3-hydroxymethyl-phenyl)-3-(naphthalen-2-ylmethoxy)-piperidine-1-carboxylate with benzoyl chloride there was obtained tert-butyl (3RS,4RS)-4-(3-benzoyloxymethyl-phenyl)-3-naphthalen-2-ylmethoxy-piperidine-1-carboxylate as a colorless oil; MS: 569 (M+NH₄)⁺.

### Example 27

The following compounds were obtained in an analogous manner to that described in Example 22(l) by cleavage of the BOC group:

1)—Methyl (3RS,4RS)-3-[3-(4-benzyloxy-naphthalen-2-ylmethoxy)-piperidin-4-yl]-benzoate as a colorless amorphous powder, MS: 482 (M+H)⁺, from tert-butyl (3RS,4RS)-3-(4-benzyloxy-naphthalen-2-ylmethoxy)-4-(3-methoxycarbonyl-phenyl)-piperidine-1-carboxylate;

2)—methyl (3RS,4RS)-3-[3-(naphthalen-2-ylmethoxy)-piperidin-4-yl]-benzoate as a colorless oil, MS: 376 (M+H)⁺, from tert-butyl (3RS,4RS)-4-(3-methoxycarbonyl-phenyl)-3-naphthalen-2-ylmethoxy-piperidine-1-carboxylate.

### Example 28

By cleavage of the BOC group from 57 mg of crude tert-butyl (3RS,4RS)-3-(4-benzyloxy-naphthalen-2-ylmethoxy)-4-(3-fluoromethyl-phenyl)-piperidine-1-carboxylate in an analogous manner to that described in Example 22 there was obtained (3RS,4RS)-3-(4-benzyloxy-naphthalen-2-ylmethoxy)-4-(3-fluoromethyl-phenyl)-piperidine as a light yellow resin; MS: 456 (M+H)⁺.

The tert-butyl (3RS,4RS)-3-(4-benzyloxy-naphthalen-2-ylmethoxy)-4-(3-fluoromethyl-phenyl)-piperidine-1-carboxylate used as the starting material was prepared as follows:

A solution of 18 mg (0.106 mmol) of diethylamino-sulfur trifluoride (DAST) in 1 ml of methylene chloride was cooled to −65° C. and a solution of 56 mg (0.101 mmol) of tert-butyl (3RS,4RS)-3-(4-benzyloxy-naphthalen-2-ylmethoxy)-4-(3-hydroxymethyl-phenyl)-piperidine-1-carboxylate in 1 ml of methylene chloride was added dropwise thereto at −60° C. to −65° C. within 3 minutes. The resulting yellow solution was warmed to room temperature and stirred for one hour. Subsequently, the mixture was partitioned between methylene chloride and 5% sodium hydrogen carbonate solution, the organic phase was dried over magnesium sulfate and finally the solvent was distilled off under reduced pressure. There were obtained 57 mg of crude tert-butyl (3RS,4RS)-3-(4-benzyloxy-naphthalen-2-ylmethoxy)-4-(3-fluoromethyl-phenyl)-piperidine-1-carboxylate, which was used in the next step without further purification and characterization.

### Example 29

(a) In an analogous manner to that described in Example 22(a), starting from 1-benzyl-4-piperidone and 2-[2-(4-bromo-phenyl)-ethoxy]-tetrahydropyran there was obtained (RS)-1-benzyl-4-[4-[2-(tetrahydro-pyran-2-yloxy)-ethyl]-phenyl]-piperidin-4-ol as a yellow oil; MS: 396 (M+H)⁺.

(b) A solution of 78 g (197 mmol) of (RS)-1-benzyl-4-[4-[2-(tetrahydro-pyran-2-yloxy)-ethyl]-phenyl]-piperidin-4-ol in 400 ml of methanol was treated with 470 ml of 2N hydrochloric acid and stirred at room temperature for 5 hours. For the work-up, the reaction solution was poured into 1500 ml of saturated sodium hydrogen carbonate solution and subsequently extracted three times with 1000 ml of ethyl acetate each time. The combined organic phases were dried over sodium sulfate and evaporated under reduced pressure. For purification of the crude substance, a flash chromatography on silica gel using a 95:5 mixture of methylene chloride and methanol as the eluent was carried out. There were obtained 51.6 g (84% of theory) of 1-benzyl-4-[4-(2-hydroxy-ethyl)-phenyl]-piperidin-4-ol as a yellowish solid; MS: 312 (M+H)⁺.

(c) In an analogous manner to that described in Example 22(b), by an elimination reaction from 1-benzyl-4-[4-(2-hydroxy-ethyl)-phenyl]-piperidin-4-ol using p-toluenesulfonic acid there was obtained 2-[4-(1-benzyl-1,2,3,6-tetrahydro-pyridin-4-yl)-phenyl]-ethanol as a colorless oil; MS: 293 (M)⁺.

(d) In an analogous manner to that described in Example 22(c), from 2-[4-(1-benzyl-1,2,3,6-tetrahydro-pyridin-4-yl)-phenyl]-ethanol by hydroboration there was obtained (3RS,4RS)-1-benzyl-4-[4-(2-hydroxy-ethyl)-phenyl]-piperidin-3-ol as a colorless oil; MS: 311 (M)⁺.

(e) In an analogous manner to that described in Example 2(e) and Example 22(g), by catalytically hydrogenating (3RS,4RS)-1-benzyl-4-[4-(2-hydroxy-ethyl)-phenyl]-piperidin-3-ol there was obtained (3RS,4RS)-4-[4-(2-hydroxy-ethyl)-phenyl]-piperidin-3-ol, which, without further purification and characterization, was converted with di-tert-butyl dicarbonate into tert-butyl (3RS,4RS)-3-hydroxy-4-[4-(2-hydroxy-ethyl)-phenyl]-piperidine-1-carboxylate; colorless oil, MS: 322 (M+H)⁺.

(f) In an analogous manner to that described in Example 22(h), from tert-butyl (3RS,4RS)-3-hydroxy-4-[4-(2-hydroxy-ethyl)-phenyl]-piperidine-1-carboxylate by introduction of the trityl group there was obtained tert-butyl (3RS,4RS)-3-hydroxy-4-[4-(2-trityloxy-ethyl)-phenyl]-piperidine-1-carboxylate as a colorless foam; MS: 581 (M+NH₄)⁺.

(g) In an analogous manner to that described in Example 22(i), from tert-butyl (3RS,4RS)-3-hydroxy-4-[4-(2-trityloxy-ethyl)-phenyl]-piperidine-1-carboxylate by alkylation with 2-bromo-methylnaphthalene there was obtained tert-butyl (3RS,4RS)-3-(naphthalen-2-ylmethoxy)-4-[4-(2-trityloxy-ethyl)-phenyl]-piperidine-1-carboxylate as a colorless oil; MS: 721 (M+H)⁺.

(h) In an analogous manner to that described in Example 22(j), from tert-butyl (3RS,4RS)-3-(naphthalen-2-ylmethoxy)-4-[4-(2-trityloxy-ethyl)-phenyl]-piperidine-1-carboxylate by cleavage of the trityl group there was obtained tert-butyl (3RS,4RS)-4-[4-(2-hydroxy-ethyl)-phenyl]-3-naphthalen-2-ylmethoxy-piperidine-1-carboxylate as a colorless solid; MS: 462 (M+H)⁺.

(i) In an analogous manner to that described in Example 22(k), by acylating tert-butyl (3RS,4RS)-4-[4-(2-hydroxy-ethyl)-phenyl]-3-naphthalen-2-ylmethoxy-piperidine-1-carboxylate with cyclopropanecarbonyl chloride there was obtained tert-butyl (3RS,4RS)-4-[4-(2-cyclopropylcarbonyloxy-ethyl)-phenyl]-3-naphthalen-2-ylmethoxy-piperidine-1-carboxylate as a colorless oil; MS: 572 (M+H)⁺.

(j) In an analogous manner to that described in Example 22(l), by cleavage of the BOC group from tert-butyl (3RS,4RS)-4-[4-(2-cyclopropylcarbonyloxy-ethyl)-phenyl]-3-naphthalen-2-ylmethoxy-piperidine-1-carboxylate using trifluoroacetic acid in methylene chloride there was obtained (3RS,4RS)-2-[4-(3-naphthalen-2-ylmethoxy-piperidin-4-yl)-phenyl]-ethyl cyclopropanecarboxylate trifluoroacetate as a white solid; MS: 430 (M+H)⁺.

### Example 30

The following compound was obtained in an analogous manner to that described in Example 23 by simultaneous cleavage of the BOC and trityl groups:

(3RS,4RS)-2-{4-[3-(naphthalen-2-ylmethoxy)-piperidin-4-yl]-phenyl}-ethanol hydrochloride as a white powder, MS: 362 (M+H)⁺, from tert-butyl (3RS,4RS)-3-(naphthalen-2-ylmethoxy)-4-[4-(2-trityloxy-ethyl)-phenyl]-piperidine-1-carboxylate.

### Example 31

The following compounds were obtained in an analogous manner to that in Example 22 (l) by cleavage of the BOC group using acid:

1)—(3RS,4RS)-2-[4-(3-Naphthalen-2-ylmethoxy-piperidin-4-yl)-phenyl]-ethyl benzoate hydrochloride as a white solid, MS: 466 (M+H)⁺, from tert-butyl (3RS,4RS)-4-[4-(2-benzoyloxy-ethyl)-phenyl]-3-(naphthalen-2-ylmethoxy)-piperidine-1-carboxylate;

2)—(3RS,4RS)-2-[4-(3-naphthalen-2-ylmethoxy-piperidin-4-yl)-phenyl]-ethyl 3-ethoxy-benzoate hydrochloride as a colorless oil, MS: 496 (M+H)⁺, from tert-butyl 3RS,4RS)-4-{4-[2-(3-methoxy-benzoyloxy)-ethyl]-phenyl}-3-(naphthalen-2-yl methoxy)-piperidine-1-carboxylate;

3)—(3RS,4RS)-2-[4-(3-naphthalen-2-ylmethoxy-piperidin-4-yl)-phenyl]-ethyl 2-methoxy-benzoate hydrochloride as a colorless solid, MS: 496 (M+H)⁺, from tert-butyl (3RS,4RS)-4-{4-[2-(2-methoxy-benzoyloxy)-ethyl]-phenyl}-3-(naphthalen-2-yl methoxy)-piperidine-1-carboxylate;

4)—(3RS,4RS)-2-[4-(3-naphthalen-2-ylmethoxy-piperidin-4-yl)-phenyl]-ethyl benzyloxy-acetate hydrochloride as a colorless solid, MS: 510 (M+H)⁺, from tert-butyl (3RS,4RS)-4-[4-(2-benzyloxyacetoxy-ethyl)-phenyl]-3-naphthalen-2-ylmethoxy-piperidine-1-carboxylate;

5)—(3RS,4RS)-2-[4-(3-naphthalen-2-yl-methoxy)-piperidin-4-yl]-phenyl]-ethyl (4-methoxy-phenyl)-acetate trifluoroacetate as a colorless solid, MS: 510 (M+H)⁺, from tert-butyl (3RS,4RS)-4-[4-[2-[(4-methoxy-phenyl)-acetoxy]-ethyl]-phenyl]-3-naphthalen-2-ylmethoxy-piperidine-1-carboxylate using trifluoroacetic acid in methylene chloride;

6)—(3RS,4RS)-2-[4-(3-naphthalen-2-ylmethoxy-piperidin-4-yl)-phenyl]-ethyl cyclohexanecarboxylate trifluoroacetate as a colorless solid, MS: 472 (M+H)⁺, from tert-butyl (3RS,4RS)-4-[4-(2-cyclohexylcarbonyloxy-ethyl)-phenyl]-3-naphthalen-2-ylmethoxy-piperidine-1-carboxylate using trifluoroacetic acid in methylene chloride;

7)—(3RS,4RS)-2-[4-(3-naphthalen-2-ylmethoxy-piperidin-4-yl)-phenyl]-ethyl 2,6-dichloro-benzoate trifluoroacetate as a colorless solid, MS: 534 (M+H)⁺, from tert-butyl (3RS,4RS)-4-{4-[2-(2,6-dichloro-benzoyloxy)-ethyl]-phenyl}-3-(naphthalen-2-ylmethoxy)-piperidine-1-carboxylate using trifluoroacetic acid in methylene chloride;

8)—(3RS,4RS)-2-[4-(3-naphthalen-2-ylmethoxy-piperidin-4-yl)-phenyl]-ethyl 2,6-dimethoxy-benzoate trifluoroacetate as a colorless solid, MS: 526 (M+H)⁺, from tert-butyl (3RS,4RS)-4-[4-[2-(2,6-dimethoxy-benzoyloxy)-ethyl]-phenyl]-3-naphthalen-2-ylmethoxy-piperidine-1-carboxylate using trifluoroacetic acid in methylene chloride;

9)—(3RS,4RS)-2-[4-(3-naphthalen-2-ylmethoxy-piperidin-4-yl)-phenyl]-ethyl 2-acetoxy-benzoate trifluoroacetate as a colorless solid, MS: 524 (M+H)⁺, from tert-butyl (3RS,4RS)-[4-[4-[2-(2-acetoxy-benzoyloxy)-ethyl]-phenyl]-3-naphthalen-2-yl methoxy-piperidine]-1-carboxylate using trifluoroacetic acid in methylene chloride;

10)—(3RS,4RS)-2-[4-(3-naphthalen-2-ylmethoxy-piperidin-4-yl)-phenyl]-ethyl 2-chloro-benzoate trifluoroacetate as a colorless solid, MS: 500 (M+H)⁺, from tert-butyl (3RS,4RS)-[4-[2-(2-chloro-benzoyloxy)-ethyl]-phenyl]-3-naphthalen-2-ylmethoxy-piperidine-1-carboxylate using trifluoroacetic acid in methylene chloride;

11)—(3RS,4RS)-2-[4-(3-naphthalen-2-ylmethoxy-piperidin-4-yl)-phenyl]-ethyl pyridin-2-yl-carbamate hydrochloride as a colorless foam, MS: 482 (M+H)⁺, from tert-butyl (3RS,4RS)-3-naphthalen-2-ylmethoxy-4-[4-(2-pyridin-2-ylcarbamoyloxy-ethyl)-phenyl]-piperidine-1-carboxylate;

12)—(3RS,4RS)-2-[4-(3-naphthalen-2-ylmethoxy-piperidin-4-yl)-phenyl]-ethyl phenyl-carbamate hydrochloride as a colorless solid, MS: 598 (M+H)⁺, from tert-butyl (3RS,4RS)-3-naphthalen-2-ylmethoxy-4-[4-(2-phenylcarbamoyloxy-ethyl)-phenyl]-piperidine-1-carboxylate;

13)—(3RS,4SR)-2-[4-(3-naphthalen-2-ylmethoxy-piperidin-4-yl)-phenyl]-ethyl pyridine-3-carboxylate hydrochloride as a colorless solid, MS: 467 (M+H)⁺, from tert-butyl (3RS,4RS)-3-naphthalen-2-ylmethoxy-4-[4-(2-pyridin-3-ylcarbonyloxy-ethyl)-phenyl]-piperidine-1-carboxylate;

14)—(3RS,4RS)-2-[4-(3-naphthalen-2-ylmethoxy-piperidin-4-yl)-phenyl]-ethyl pyridine-2-carboxylate as a colorless oil, MS: 467 (M+H)⁺, from 2-{4-[1-tert-butoxycarbonyl-3-(naphthalen-2-ylmethoxy)-piperidin-4-yl]-phenyl}-ethyl (3RS,4RS)-pyridine-2-carboxylate;

15)—(3RS,4RS)-2-[4-(3-naphthalen-2-ylmethoxy-piperidin-4-yl)-phenyl]-ethyl thiophene-3-carboxylate trifluoroacetate as a colorless solid, MS: 472 (M+H)⁺, from tert-butyl (3RS,4RS)-3-naphthalen-2-ylmethoxy-4-[4-(2-thiophen-3-ylcarbonyloxy-ethyl)-phenyl]-piperidine-1-carboxylate using trifluoroacetic acid in methylene chloride;

16)—(3RS,4RS)-2-[4-(3-naphthalen-2-ylmethoxy-piperidin-4-yl)-phenyl]-ethyl thiophen-2-ylacetate trifluoroacetate as a colorless solid, MS: 486 (M+H)⁺, from tert-butyl (3RS,4RS)-3-naphthalen-2-ylmethoxy-4-[4-(2-thiophen-3-ylacetoxy-ethyl)-phenyl]-piperidine-1-carboxylate using trifluoroacetic acid in methylene chloride;

17)—(3RS,4RS)-2-[4-(3-naphthalen-2-ylmethoxy-piperidin-4-yl)-phenyl]-ethyl imidazole-2-carboxylate trifluoroacetate as a colorless solid, MS: 456 (M+H)⁺, from tert-butyl (3RS,4RS)-4-[4-(2-imidazol-2-ylcarbonyloxy-ethyl)-phenyl]-3-naphthalen-2-ylmethoxy]-piperidine-1-carboxylate using trifluoroacetic acid in methylene chloride;

18)—(3RS,4RS)-2-[4-[3-(4-benzyloxy-naphthalen-2-ylmethoxy)-piperidin-4-yl]-phenyl]-ethyl benzoate hydrochloride as a yellowish solid, MS: 572 (M+H)⁺, from tert-butyl (3RS,4RS)-4-[4-(2-benzyloxy-ethyl)-phenyl-3-(4-benzyloxy-naphthalen-2-ylmethoxy)-piperidine-1-carboxylate.

The following starting materials were obtained analogously to the procedure described in Example 22 (k):

(a) tert-Butyl (3RS,4RS)-4-[4-(2-benzoyloxy-ethyl)-phenyl]-3-(naphthalen-2-ylmethoxy)-piperidine-1-carboxylate as a colorless oil, MS: 583 (M+NH₄)⁺, from tert-butyl (3RS,4RS)-4-[4-(2-hydroxy-ethyl)-phenyl]-3-naphthalen-2-ylmethoxy-piperidine-1-carboxylate and benzoyl chloride;

(b) tert-butyl (3RS,4RS)-4-{4-[2-(3-methoxy-benzoyloxy)-ethyl]-phenyl}-3-(naphthalen-2-yl methoxy)-piperidine-1-carboxylate as a colorless oil, MS: 593 (M+H)⁺, from tert-butyl (3RS,4RS)-4-[4-(2-hydroxy-ethyl)-phenyl]-3-naphthalen-2-ylmethoxy-piperidine-1-carboxylate and 3-methoxybenzoyl chloride;

(c) tert-butyl (3RS,4RS)-4-{4-[2-(2-methoxy-benzoyloxy)-ethyl]-phenyl}-3-(naphthalen-2-yl methoxy)-piperidine-1-carboxylate as a colorless oil, R_{f}: 0.35 (SiO₂, hexane:ethyl acetate=2:1), from tert-butyl (3RS,4RS)-4-[4-(2-hydroxy-ethyl)-phenyl]-3-naphthalen-2-ylmethoxy-piperidine-1-carboxylate and 2-methoxybenzoyl chloride;

(d) tert-butyl (3RS,4RS)-4-[4-(2-benzyloxyacetoxy-ethyl)-phenyl]-3-naphthalen-2-ylmethoxy-piperidine-1-carboxylate as a colorless oil, MS: 627 (M+NH₄)⁺, from tert-butyl (3RS,4RS)-4-[4-(2-hydroxy-ethyl)-phenyl]-3-naphthalen-2-ylmethoxy-piperidine-1-carboxylate and benzyloxyacetyl chloride;

(e) tert-butyl (3RS,4RS)-4-[4-[2-[(4-methoxy-phenyl)-acetoxy]-ethyl]-phenyl]-3-naphthalen-2-ylmethoxy-piperidine-1-carboxylate as a colorless oil, MS: 627 (M+NH₄)⁺, from tert-butyl (3RS,4RS)-4-[4-(2-hydroxy-ethyl)-phenyl]-3-naphthalen-2-ylmethoxy-piperidine-1-carboxylate and 4-methoxyphenylacetyl chloride;

(f) tert-butyl (3RS,4RS)-4-[4-(2-cyclohexylcarbonyloxy-ethyl)-phenyl]-3-naphthalen-2-ylmethoxy-piperidine-1-carboxylate as a colorless oil, MS: 572 (M+H)⁺, from tert-butyl (3RS,4RS)-4-[4-(2-hydroxy-ethyl)-phenyl]-3-naphthalen-2-ylmethoxy-piperidine-1-carboxylate and cyclohexanecarbonyl chloride;

(g) tert-butyl (3RS,4RS)-4-{4-[2-(2,6-dichloro-benzoyloxy)-ethyl]-phenyl}-3-(naphthalen-2-ylmethoxy)-piperidine-1-carboxylate as a colorless oil, MS: 651 (M+NH₄)⁺, from tert-butyl (3RS,4RS)-4-[4-(2-hydroxy-ethyl)-phenyl]-3-naphthalen-2-ylmethoxy-piperidine carboxylate and 2,6-dichlorobenzoyl chloride;

(h) tert-butyl (3RS,4RS)-4-[4-[2-(2,6-dimethoxy-benzoyloxy)-ethyl]-phenyl]-3-naphthalen-2-ylmethoxy-piperidine-1-carboxylate as a colorless amorphous solid, MS: 643 (M+NH₄)⁺, from tert-butyl (3RS,4RS)-4-[4-(2-hydroxy-ethyl)-phenyl]-3-naphthalen-2-ylmethoxy-piperidine-1-carboxylate and 2,6-dimethoxybenzoyl chloride;

(i) tert-butyl (3RS,4RS)-[4-[4-[2-(2-acetoxy-benzoyloxy)-ethyl)-phenyl]-3-naphthalen-2-ylmethoxy-piperidine]-1-carboxylate as a colorless oil, MS: 641 (M+NH₄)⁺, from tert-butyl (3RS,4RS)-4-[4-(2-hydroxy-ethyl)-phenyl]-3-naphthalen-2-ylmethoxy-piperidine-1-carboxylate and O-acetylsalicyloyl chloride;

(j) tert-butyl (3RS,4RS)-[4-[2-(2-chloro-benzoyloxy)-ethyl]-phenyl]-3-naphthalen-2-ylmethoxy-piperidine-1-carboxylate as a colorless oil, MS: 617 (M+NH₄)⁺; from tert-butyl (3RS,4RS)-4-[4-(2-hydroxy-ethyl)-phenyl]-3-naphthalen-2-ylmethoxy-piperidine-1-carboxylate and 2-chlorobenzoyl chloride;

(k) In an analogous manner to that described in Example 24(m), by reacting tert-butyl (3RS,4RS)-4-[4-(2-hydroxy-ethyl)-phenyl]-3-naphthalen-2-ylmethoxy-piperidine-1-carboxylate with pyridine-2-carbonyl azide there was obtained tert-butyl (3RS,4RS)-3-naphthalen-2-ylmethoxy-4-[4-(2-pyridin-2-ylcarbamoyloxy-ethyl)-phenyl]-piperidine-1-carboxylate as colorless crystals, MS: 582 (M+H)⁺.

(l) In an analogous manner to that described in Example 24(m), by reacting tert-butyl (3RS,4RS)-4-[4-(2-hydroxy-ethyl)-phenyl]-3-naphthalen-2-ylmethoxy-piperidine-1-carboxylate with phenyl isocyanate there was obtained tert-butyl (3RS,4RS)-3-naphthalen-2-ylmethoxy-4-[4-(2-phenylcarbamoyloxy-ethyl)-phenyl]-piperidine-1-carboxylate as a colorless oil, MS: 481 (M+H)⁺.

(m) In an analogous manner to that described in Example 24(g), by condensing tert-butyl (3RS,4RS)-4-[4-(2-hydroxy-ethyl)-phenyl]-3-naphthalen-2-ylmethoxy-piperidine-1-carboxylate and nicotinic acid there was obtained tert-butyl (3RS,4RS)-3-naphthalen-2-ylmethoxy-4-[4-(2-pyridin-3-ylcarbonyloxy-ethyl)-phenyl]-piperidine-1-carboxylate as a colorless oil, MS: 567 (M+H)⁺.

(n) In an analogous manner to that described in Example 24(g), by condensing tert-butyl (3RS,4RS)-4-[4-(2-hydroxy-ethyl)-phenyl]-3-naphthalen-2-ylmethoxy-piperidine-1-carboxylate and pyridine-2-carboxylic acid using 1,1-carbonyldiimidazole as the condensation agent there was obtained 2-{4-[1-tert-butoxycarbonyl-3-(naphthalen-2-ylmethoxy)-piperidin-4-yl]-phenyl}-ethyl (3RS,4RS)-pyridine-2-carboxylate as a colorless oil, MS: 467 (M+H)⁺.

(o) In an analogous manner to that described in Example 24(g), by condensing tert-butyl (3RS,4RS)-4-[4-(2-hydroxy-ethyl)-phenyl)-3-naphthalen-2-ylmethoxy-piperidine-1-carboxylate and thiophene-3-carboxylic acid using 1,1-carbonyldiimidazole as the condensation agent there was obtained tert-butyl (3RS,4RS)-3-naphthalen-2-ylmethoxy-4-[4-(2-thiophen-3-ylcarbonyloxy-ethyl)-phenyl]-piperidine-1-carboxylate as white crystals, MS: 572 (M+H)⁺.

(p) In an analogous manner to that described in Example 24(g), by condensing tert-butyl (3RS,4RS)-4-[4-(2-hydroxy-ethyl)-phenyl)-3-naphthalen-2-ylmethoxy-piperidine-1-carboxylate and 3-thiopheneacetic acid using 1,1-carbonyldiimidazole as the condensation agent there was obtained tert-butyl (3RS,4RS)-3-naphthalen-2-ylmethoxy-4-[4-(2-thiophen-3-ylacetoxy-ethyl)-phenyl]-piperidine-1-carboxylate as a colorless solid, MS: 603 (M+NH₄)⁺.

(q) In an analogous manner to that described in Example 24(g), by condensing tert-butyl (3RS,4RS)-4-[4-(2-hydroxy-ethyl)-phenyl]-3-naphthalen-2-ylmethoxy-piperidine 1-carboxylate and imidazole-2-carboxylic acid using 1,1-carbonyldiimidazole as the condensation agent there was obtained tert-butyl (3RS,4RS)-4-[4-(2-imidazol-2-ylcarbonyloxy-ethyl)-phenyl]-3-naphthalen-2-ylmethoxy]-piperidine-1-carboxylate as a colorless solid, MS: 556 (M+H)⁺.

The following procedure was carried out in an analogous manner to that described in Example 22(i)-(k):

(r) Alkylation of tert-butyl (3RS,4RS)-3-hydroxy-4-[4-(2-trityloxy-ethyl)-phenyl]-piperidine-1-carboxylate with 1-benzyloxy-3-chloromethyl-naphthalene gave tert-butyl (3RS,4RS)-3-(4-benzyloxy-naphthalen-2-ylmethoxy)-4-[4-(2-trityloxy-ethyl)-phenyl]-piperidine-1-carboxylate as a colorless oil, MS: 827 (M+NH₄)⁺.

(s) Cleavage of the trityl group from tert-butyl (3RS,4RS)-3-(4-benzyloxy-naphthalen-2-ylmethoxy)-4-[4-(2-trityloxy-ethyl)-phenyl]-piperidine-1-carboxylate yielded tert-butyl (3RS,4RS)-3-(4-benzyloxy-naphthalen-2-ylmethoxy)-4-[4-(2-hydroxy-ethyl)-phenyl]-piperidine-1-carboxylate as a colorless oil, MS: 568 (M+H)⁺.

(t) Acylation of tert-butyl (3RS,4RS)-3-(4-benzyloxy-naphthalen-2-ylmethoxy)-4-[4-(2-hydroxy-ethyl)-phenyl]-piperidine-1-carboxylate with benzoyl chloride gave (3RS,4RS)-4-[4-(2-benzyloxy-ethyl)-phenyl-3-(4-benzyloxy-naphthalen-2-ylmethoxy)-piperidine-1-carboxylate as a colorless oil, MS: 689 (M+NH₄)⁺.

### Example 32

The following compounds were obtained in an analogous manner to that described in Example 22(l) by cleavage of the BOC group using acid:

1)—(3RS,4RS)-3-Naphthalen-2-ylmethoxy-4-(4-naphthalen-2-ylmethoxymethyl-phenyl)-piperidine hydrochloride as a colorless solid, MS: 488 (M+H)⁺, from tert-butyl (3RS,4RS)-3-(naphthalen-2-ylmethoxy)-4-[4-(naphthalen-2-ylmethoxymethyl)-phenyl]-piperidine-1-carboxylate;

2)—(3RS,4RS)-4-[4-(4-methoxy-phenoxymethyl)-phenyl]-3-naphthalen-2-ylmethoxy-piperidine as a yellow foam, MS: 454 (M+H)⁺, from tert-butyl (3RS,4RS)-4-[4-(4-methoxy-phenoxy-methyl)-phenyl]-3-naphthalen-2-ylmethoxy-piperidine-1-carboxylate;

3)—(3RS,4RS)-3-(1-methoxy-naphthalen-2-ylmethoxy)-4-[4-(2-phenoxy-ethyl)-phenyl]-piperidine trifluoroacetate as a colorless oil, MS: 468 (M+H)⁺, from tert-butyl (3RS,4RS)-3-(1-methoxy-naphthalen-2-ylmethoxy)-4-[4-(2-phenoxy-ethyl)-phenyl]-piperidine-1-carboxylate using trifluoroacetic acid in methylene chloride;

4)—(3RS,4RS)-4-[4-(2-methoxy-ethyl)-phenyl]-3-naphthalen-2-ylmethoxy]-piperidine as a colorless solid, MS: 376 (M+H)⁺, from tert-butyl (3RS,4RS)-4-[4-(2-methoxy-ethyl)-phenyl]-3-naphthalen-2-ylmethoxy]-piperidine-1-carboxylate.

The BOC derivatives use as the starting materials were prepared as follows:

(a) In an analogous manner to that described in Example 22(i), by alkylating tert-butyl (3RS,4RS)-3-hydroxy-4-(4-hydroxymethyl-phenyl)-piperidine-1-carboxylate with 2-bromomethylnaphthalene there was obtained tert-butyl (3RS,4RS)-3-(naphthalen-2-ylmethoxy)-4-[4-(naphthalen-2-ylmethoxymethyl)-phenyl]-piperidine-1-carboxylate as a colorless oil, R.sub.f: 0.31 (SiO₂, hexane:ethyl acetate=2:1).

b) A solution of 200 mg (0.45 mmol) of tert-butyl (3RS,4RS)-4-(4-hydroxymethyl-phenyl)-3-(naphthalen-2-ylmethoxy)-piperidine-1-carboxylate in 10 ml of tetrahydrofuran was treated in sequence with 143 mg (0.58 mmol) of triphenylphosphine, 94 mg (0.58 mmol) of diethylazo dicarboxylate and 166 mg (1.35 mmol) of hydroquinone monomethyl ether and the reaction mixture was stirred at room temperature for 15 hours. For the working-up, the reaction mixture was diluted with 20 ml of methylene chloride and extracted with 20 ml of saturated sodium carbonate solution. The organic phase was dried over sodium sulfate and evaporated under reduced pressure. For purification, the crude product was chromatographed on silica gel using methylene chloride as the eluent. There were obtained 245 mg of colorless oil which contained tert-butyl (3RS,4RS)-4-(4-hydroxymethyl-phenyl)-3-(naphthalen-2-yl-methoxy)-piperidine-1-carboxylate in addition to tert-butyl (3RS,4RS)-4-[4-(4-methoxy-phenoxymethyl)-phenyl]-3-naphthalen-2-ylmethoxy-piperidine-1-carboxylate, R_{f}: 0.08 (methylene chloride).

The following procedure was carried out in an analogous manner to that described in Example 22(i)-(j):

(c) Alkylation of tert-butyl (3RS,4RS)-3-hydroxy-4-[4-(2-trityloxy-ethyl)-phenyl]-piperidine-1-carboxylate with 2-bromomethyl-1-methoxy-naphthalene gave tert-butyl (3RS,4RS)-3-(1-methoxy-naphthalen-2-ylmethoxy)-4-[4-(2-trityloxy-ethyl)-phenyl]-piperidine-1-carboxylate from which, after cleavage of the trityl group there was obtained tert-butyl (3RS,4RS)-4-[4-(2-hydroxy-ethyl)-phenyl]-3-(1-methoxy-naphthalen-2-ylmethoxy)-piperidine-1-carboxylate and, after reaction with phenol, analogously to the procedure described under (b), tert-butyl (3RS,4RS)-3-(1-methoxy-naphthalen-2-ylmethoxy)-4-[4-(2-phenoxy-ethyl)-phenyl]-piperidine-1-carboxylate as a colorless oil; R_{f}: 0.34 (SiO₂, hexane:ethyl acetate=2:1).

(d) in an analogous manner to that described in Example 22(i) by alkylating tert-butyl (3RS,4RS)-4-[4-(2-hydroxy-ethyl)-phenyl]-3-naphthalen-2-ylmethoxy-piperidine-1-carboxylate with methyl iodide there was obtained tert-butyl (3RS,4RS)-4-[4-(2-methoxy-ethyl)-phenyl]-3-naphthalen-2-ylmethoxy]-piperidine-1-carboxylate as a colorless oil; MS: 493 (M+NH₄)⁺.

### Example 33

The following compounds were obtained in an analogous manner to that described in Example 22 (l) by cleavage of the BOC group by means of acid:

1)—(3RS,4RS)-3-naphthalen-2-ylmethoxy-4-[4-(2-phenyl-sulfanyl-ethyl)-phenyl]-piperidine hydrochloride as white crystals, MS: 454 (M+H)⁺, from tert-butyl (3RS,4RS)-3-naphthalen-2-ylmethoxy-4-[4-(2-phenylsulfanyl-ethyl)-phenyl]-piperidine-1-carboxylate;

2)—a mixture of (3RS,4RS)-3-naphthalen-2-ylmethoxy-4-[4-[(RS)- and -[(SR)-2-phenylsulfinyl-ethyl]-phenyl]-piperidine as a colorless amorphous solid, MS: 470 (M+H)⁺, from a mixture of tert-butyl (3RS,4RS)-3-naphthalen-2-ylmethoxy-4-[4-[(RS)-and-[(SR)-2-phenylsulfinyl-ethyl]-phenyl]-piperidine-1-carboxylate;

3)—(3RS,4RS)-3-naphthalen-2-ylmethoxy-4-[4-(2-phenyl-sulfonyl-ethyl)-phenyl]-piperidine hydrochloride as white crystals, MS: 486 (M+H)⁺, from tert-butyl (3RS,4RS)-3-naphthalen-2-ylmethoxy-4-[4-(2-phenylsulfonyl-ethyl)-phenyl]-piperidine-1-carboxylate;

4)—(3RS,4RS)-4-[4-(2-benzothiazol-2-ylsulfanyl-ethyl)-phenyl]-3-naphthalen-2-ylmethoxy-piperidine as a colorless foam, MS: 511 (M+H)⁺, from tert-butyl (3RS,4RS)-4-[4-(2-benzothiazol-2-ylsulfanyl-ethyl)-phenyl]-3-naphthalen-2-ylmethoxy-piperidine-1-carboxylate;

5)—(3RS,4RS)-2-[4-(3-naphthalen-2-ylmethoxy-piperidin-4-yl)-benzylsulfanyl]-benzothiazole as a yellow foam, MS: 497 (M+H)⁺, from tert-butyl (3RS,4RS)-4-[4-(benzothiazol-2-ylsulfanylmethyl)-phenyl]-3-naphthalen-2-ylmethoxy-piperidine-1-carboxylate;

6)—(3RS,4RS)-3-naphthalen-2-ylmethoxy-4-[4-(1-phenyl-1H-tetrazol-5-ylsulfanylmethyl)-phenyl]-piperidine hydrochloride as a colorless oil, MS: 508 (M+H)⁺, from tert-butyl (3RS,4RS)-3-naphthalen-2-ylmethoxy-4-[4-(1-phenyl-1H-tetrazol-5-ylsulfanylmethyl)-phenyl]-piperidine-1-carboxylate;

7)—(3RS,4RS)-3-(naphthalen-2-ylmethoxy)-4-[4-(2-phenoxy-ethyl)-phenyl]-piperidine as a yellowish foam, MS: 438 (M+H)⁺, from tert-butyl (3RS,4RS)-3-(naphthalen-2-ylmethoxy)-4-[4-(2-phenoxy-ethyl)-phenyl]-piperidine-1-carboxylate.

The BOC derivatives used as the starting materials were prepared as follows:

(a) A mixture of 200 mg (0.43 mmol) of tert-butyl (3RS,4RS)-4-[4-(2-hydroxy-ethyl)-phenyl]-3-naphthalen-2-ylmethoxy-piperidine-1-carboxylate, 263 mg (1.29 mmol) of tributylphosphine and 284 mg (1.29 mmol) of diphenyl sulfide in 1 ml of pyridine was stirred at room temperature for 18 hours. Subsequently, the reaction mixture was evaporated under reduced pressure and the residue, without further working-up, was chromatographed directly on silica gel using methylene chloride as the eluent. The tert-butyl (3RS,4RS)-3-naphthalen-2-ylmethoxy-4-[4-(2-phenylsulfanyl-ethyl)-phenyl]-piperidine-1-carboxylate was obtained as a colorless oil in quantitative yield; MS: 554 (M+H)⁺.

(b) A solution of 216 mg (0.23 mmol) of tetrabutylammonium ozone was added dropwise very slowly at room temperature to a solution of 128 mg (0.23 mmol) of tert-butyl (3RS,4RS)-3-naphthalen-2-ylmethoxy-4-[4-(2-phenylsulfanyl-ethyl)-phenyl)-piperidine-1-carboxylate in 10 ml of methylene chloride. After 6 hours the reaction solution was evaporated under reduced pressure and the crude product was chromatographed on silica gel using a 2:1 mixture of hexane and ethyl acetate as the eluent in order to separate the sulfone which had already formed. There were obtained 100 mg (76% of theory) of a mixture of tert-butyl (3RS,4RS)-3-naphthalen-2-ylmethoxy-4-[4-[(RS)- and -[(SR)-2-phenylsulfinyl-ethyl]-phenyl]-piperidine-1-carboxylate as a colorless oil which gradually crystallized out; MS: 570 (M+H)⁺.

(c) In an analogous manner to that described under (b), starting from tert-butyl (3RS,4RS)-3-naphthalen-2-ylmethoxy-4-[4-(2-phenylsulfanyl-ethyl)-phenyl]-piperidine-1-carboxylate using an excess of tetrabutylammonium oxone there was obtained tert-butyl (3RS,4RS)-3-naphthalen-2-ylmethoxy-4-[4-(2-phenylsulfonyl-ethyl)-phenyl]-piperidine-1-carboxylate as a colorless foam; MS: 603 (M+NH₄)⁺.

(d) In an analogous manner to that described under (a), by reacting tert-butyl (3RS,4RS)-4-[4-(2-hydroxy-ethyl)-phenyl]-3-naphthalen-2-ylmethoxy-piperidine-1-carboxylate with bis-(benzothiazol-2-yl) disulfide there was obtained tert-butyl (3RS,4RS)-4-[4-(2-benzothiazol-2-ylsulfanyl-ethyl)-phenyl]-3-naphthalen-2-ylmethoxy-piperidine-1-carboxylate as a yellowish solid; MS: 611 (M+H)⁺.

(e) In an analogous manner to that described under (a), by reacting tert-butyl (3RS,4RS)-4-(4-hydroxymethyl-phenyl)-3-(naphthalen-2-ylmethoxy)-piperidine-1-carboxylate with (benzothiazol-2-yl) disulfide there was obtained tert-butyl (3RS,4RS)-4-[4-(benzothiazol-2-ylsulfanylmethyl)-phenyl]-3-naphthalen-2-ylmethoxy-piperidine-1-carboxylate as a yellowish foam; MS: 597 (M+H)⁺.

(f) In an analogous manner to that described under (a), by reacting tert-butyl (3RS,4RS)-4-(4-hydroxymethyl-phenyl)-3-(naphthalen-2-ylmethoxy)-piperidine-1-carboxylate with bis-(1-phenyl-1H-tetrazol-5-yl) disulfide there was obtained tert-butyl (3RS,4RS)-3-naphthalen-2-ylmethoxy-4-[4-(1-phenyl-1H-tetrazol-5-ylsulfanylmethyl)-phenyl]-piperidine-1-carboxylate as a colorless foam; MS: 630 (M+Na)⁺.

(g) In an analogous manner to that described in Example 32(b), from tert-butyl (3RS,4RS)-4-[4-(2-hydroxy-ethyl)-phenyl]-3-naphthalen-2-ylmethoxy-piperidine-1-carboxylate there was obtained tert-butyl (3RS,4RS)-3-(naphthalen-2-ylmethoxy)-4-[4-(2-phenoxy-ethyl)-phenyl]-piperidine-1-carboxylate as a colorless oil; MS: 555 (M+NH₄)⁺.

### Example 34

(a) To a solution of 100 mg (0.22 mmol) of tert-butyl (3RS,4RS)-4-(4-hydroxymethyl-phenyl)-3-(naphthalen-2-ylmethoxy)-piperidine-1-carboxylate in 10 ml of tetrahydrofuran were added 68 mg (0.66 mmol) of triethylamine and thereafter dropwise at 0° C. 25 mg (0.26 mmol) of methanesulfonyl chloride. The reaction solution was stirred at room temperature for 90 minutes, thereafter 38 mg (0.33 mmol) of 2-mercaptopyrimidine were added and the mixture was stirred at room temperature for a further 18 hours. For the working-up, the reaction mixture was evaporated under reduced pressure, the residue was taken up in 20 ml of methylene chloride and then extracted with 10 ml of water. The organic phase was dried over sodium sulfate and subsequently evaporated under reduced pressure. For purification, the crude product was chromatographed on silica gel using a 5:1 mixture of hexane and ethyl acetate as the eluent. There were obtained 100 mg (82% of theory) of tert-butyl (3RS,4RS)-3-naphthalen-2-ylmethoxy-4-[4-(pyrimidin-2-ylsulfanylmethyl)-phenyl]-piperidine-1-carboxylate as a yellowish oil; MS: 542 (M+H)⁺.

(b) In an analogous manner to that described in Example 22(l), by cleavage of the BOC group there was obtained (3RS,4RS)-2-[4-(3-naphthalen-2-ylmethoxy-piperidin-4-yl)-benzylsulfanyl]-pyrimidine as a yellow foam; MS: 442 (M+H)⁺.

### Example 35

The following compounds were obtained in an analogous manner to that described in Example 22(l) by cleavage of the BOC group using acid:

1)—(3RS,4RS)-2-[4-(3-naphthalen-2-ylmethoxy-piperidin-4-yl)-benzylsulfanyl]-6-nitro-benzothiazole hydrochloride as a yellowish foam, MS: 542 (M+H)⁺, from tert-butyl (3RS,4RS)-3-naphthalen-2-ylmethoxy-4-[4-(6-nitro-benzothiazol-2-ylsulfanylmethyl)-phenyl]-piperidine-1-carboxylate;

2)—(3RS,4RS)-3-{4-[3-(naphthalen-2-ylmethoxy)-piperidin-4-yl]-phenyl}-propionitrile trifluoroacetate as a white powder, MS: 371 (M+H)⁺, from tert-butyl (3RS,4RS)-4-[4-(2-cyano-ethyl)-phenyl]-3-naphthalen-2-ylmethoxy-piperidine-1-carboxylate using trifluoroacetic acid in methylene chloride.

The BOC derivatives used as the starting materials were obtained as follows:

(a) In an analogous manner to that described in Example 34(a), by reacting tert-butyl (3RS,4RS)-4-(4-hydroxymethyl-phenyl)-3-(naphthalen-2-ylmethoxy)-piperidine-1-carboxylate with 6-nitro-2-mercaptobenzothiazole there was obtained, via the mesylate prepared in situ, tert-butyl (3RS,4RS)-3-naphthalen-2-ylmethoxy-4-[4-(6-nitro-benzothiazol-2-ylsulfanylmethyl) phenyl]-piperidine-1-carboxylate as a yellow solid, MS: 642 (M+H)⁺.

(b) In an analogous manner to that described in Example 34(a), by reacting tert-butyl (3RS,4RS)-4-[4-(2-hydroxy-ethyl)-phenyl]-3-naphthalen-2-ylmethoxy-piperidine-1-carboxylate with potassium cyanide in dimethylformamide there was obtained, via the corresponding mesylate, tert-butyl (3RS,4RS)-4-[4-(2-cyano-ethyl)-phenyl]-3-naphthalen-2-ylmethoxy-piperidine-1-carboxylate as a white powder, MS: 471 (M+H)⁺.

### Example 36

(a) 6.95 g (44 mmol) of powdered potassium permanganate dissolved in a mixture of 100 ml of water and 100 ml of glacial acetic acid as well as 0.73 g (2 mmol) of tetrabutylammonium iodide were added to a solution of 5.0 g (10.83 mmol) of tert-butyl (3RS,4RS)-4-[4-(2-hydroxy-ethyl)-phenyl]-3-naphthalen-2-ylmethoxy-piperidine-1-carboxylate in 500 ml of benzene. The reaction mixture was stirred intensively for 48 hours. For the working-up, the phases were separated. The organic phase was washed with 100 ml of saturated sodium thiosulfate solution. The aqueous phase was decolorized by the addition of saturated sodium thiosulfate solution and subsequently extracted twice with 100 ml of ethyl acetate and 100 ml of methylene chloride each time. The organic phases were combined, dried over sodium sulfate and evaporated under reduced pressure. For purification, the crude material was chromatographed on silica gel using a 9:1 mixture of methylene chloride and methanol as the eluent after the column had previously been prepared with a 90:10:0.1 mixture of methylene chloride, methanol and ammonia. There were obtained 2.6 g (50% of theory) of (3RS,4RS)-[4-(1-tert-butoxycarbonyl-3-naphthalen-2-ylmethoxy-piperidin-4-yl)-phenyl]-acetic acid as a colorless, amorphous solid; MS: 476 (M+H)⁺.

(b) A solution of 150 mg (0.32 mmol) of (3RS,4RS)-[4-(1-tert-butoxycarbonyl-3-naphthalen-2-ylmethoxy-piperidin-4-yl)-phenyl]-acetic acid and 55 mg (0.32 mmol) of 2-amino-1-phenyl-ethanone in 5 ml of dimethylformamide was treated in sequence with 44.6 μl (0.32 mmol) of triethylamine and 96 mg (0.32 mmol) of O-(1,2-dihydro-2-oxo-1-pyridyl)-N,N,N′,N′-tetramethyluronium tetrafluoroborate (TPTU) and the mixture was stirred at room temperature for 18 hours. For the working-up, the reaction was evaporated in an oil pump vacuum, the residue was taken up in 20 ml of methylene chloride and washed with 5 ml of water. The organic phase was dried over sodium sulfate and evaporated under reduced pressure. For purification, the crude material was chromatographed on silica gel using a 95:5 mixture of methylene chloride and methanol as the eluent. There were obtained 120 mg (64% of theory) of tert-butyl (3RS,4RS)-3-(naphthalen-2-ylmethoxy)-4-{4-[(2-oxo-2-phenyl-ethylcarbamoyl)-methyl]-phenyl}-piperidine-1-carboxylate as a colorless oil; MS: 615 (M+Na)⁺.

(c) In an analogous manner to that described in Example 22(l), starting from tert-butyl (3RS,4RS)-3-(naphthalen-2-ylmethoxy)-4-{4-[(2-oxo-2-phenyl-ethylcarbamoyl)-methyl]-phenyl}-piperidine-1-carboxylate by cleavage of the BOC group using trifluoroacetic acid in methylene chloride there was obtained (3RS,4RS)-2-{4-[3-(naphthalen-2-ylmethoxy)-piperidin-4-yl]-phenyl}-N-(2-oxo-2-phenyl-ethyl)-acetamide trifluoroacetate as a white powder; MS: 493 (M+H)⁺.

### Example 37

The following compounds were obtained in an analogous manner to that described in Example 36(b)-(c):

1)—From (3RS,4RS)-[4-(1-tert-butoxycarbonyl-3-naphthalen-2-ylmethoxy-piperidin-4-yl)-phenyl]-acetic acid and 3-amino-1-phenyl-propan-1-one [H. Zinner and G. Brossmann, J. Prakt. Chem. 5, 91 (1958)] there was obtained tert-butyl (3RS,4RS)-3-(naphthalen-2-ylmethoxy)-4-{4-[(3-oxo-3-phenyl-propylcarbamoyl)-methyl]-phenyl}-piperidine-1-carboxylate, MS: 607 (M+H)⁺, 15154B66, which, after cleavage of the BOC group, gave (3RS,4RS)-2-[4-[3-(naphthalen-2-ylmethoxy)-piperidin-4-yl]-phenyl]-N-(3-oxo-3-phenyl-propyl)-acetamide trifluoroacetate as a white powder; MS: 507 (M+H)⁺;

2)—from (3RS,4RS)-[4-(1-tert-butoxycarbonyl-3-naphthalen-2-ylmethoxy-piperidin-4-yl)-phenyl]-acetic acid and 2-hydroxy-1-phenyl-ethanone with 1,1-carbonyldiimidazole as the condensation agent there was obtained tert-butyl (3RS,4RS)-3-(naphthalen-2-ylmethoxy)-4-[4-(2-oxo-2-phenyl-ethoxycarbonylmethyl)-phenyl]-piperidine-1-carboxylate which, after cleavage of the BOC group, gave 2-oxo-2-phenyl-ethyl (3RS,4RS)-[4-[3-(naphthalen-2-ylmethoxy)-piperidin-4-yl]-phenyl]-acetate trifluoroacetate as a white powder; MS: 494 (M+H)⁺;

3)—from (3RS,4RS)-[4-(1-tert-butoxycarbonyl-3-naphthalen-2-ylmethoxy-piperidin-4-yl)-phenyl]-acetic acid and phenol with 1,1-carbonyldiimidazole as the condensation agent there was obtained tert-butyl (3RS,4RS)-3-(naphthalen-2-ylmethoxy)-4-(4-phenoxycarbonylmethyl-phenyl)-piperidine-1-carboxylate which, after cleavage of the BOC group, gave phenyl (3RS,4RS)-[4-(3-naphthalen-2-ylmethoxy-piperidin-4-yl)-phenyl]-acetate trifluoroacetate as white crystals; MS: 452 (M+H)⁺.

### Example 38

(a) A solution of 100 mg (0.21 mmol) of (3RS,4RS)-[4-(1-tert-butoxycarbonyl-3-naphthalen-2-ylmethoxy-piperidin-4-yl)-phenyl]-acetic acid in 5 ml of dimethylformamide was treated with 50 mg (0.31 mmol) of 1,1-carbonyldiimidazole and the mixture was stirred at 50° C. for one hour. Subsequently, the mixture was cooled to room temperature, a solution of 45 mg (0.33 mmol) of benzamidoxime in 2 ml of dimethylformamide was added and the mixture was stirred at 50° C. for one hour. For the working-up, the mixture was cooled to room temperature and the dimethylformamide was distilled off in an oil pump vacuum. The residue was chromatographed on silica gel using a 98:2:0.1 mixture of methylene chloride, methanol and ammonia as the eluent. There were obtained 90 mg (72% of theory) of the benzamidoxime ester as a colorless foam; MS: 594 (M+H)⁺, R_{f}: 0.68 (SiO₂, methylene chloride:methanol:ammonia=95:5:0.1)

(b) A solution of 90 mg (0.15 mmol) of the benzamidoxime ester in 10 ml of dimethylformamide was heated to 130° C. for 18 hours. For the working-up, the mixture was evaporated under reduced pressure and the residue was chromatographed on silica gel using a 4:1 mixture of hexane and ethyl acetate as the eluent. There were obtained 63 mg (72% of theory) of tert-butyl (3RS,4RS)-3-(naphthalen-2-ylmethoxy)-4-[4-(3-phenyl-[1,2,4]oxadiazol-5-ylmethyl)-phenyl]-piperidine-1-carboxylate as a colorless oil; MS: 592 (M+NH₄)⁺.

(c) In an analogous manner to that described in Example 22(l), starting from tert-butyl (3RS,4RS)-3-(naphthalen-2-ylmethoxy)-4-[4-(3-phenyl-[1,2,4]oxadiazol-5-ylmethyl)-phenyl]-piperidine-1-carboxylate by cleavage of the BOC group using trifluoroacetic acid in methylene chloride there was obtained (3RS,4RS)-3-(3-naphthalen-2-ylmethoxy)-4-[4-(3-phenyl-[1,2,4]oxadiazol-5-ylmethyl)-phenyl]-piperidine trifluoroacetate as a white powder; MS: 476 (M+H)⁺.

### Example 39

(a) A solution of 100 mg (0.21 mmol) of (3RS,4RS)-[4-(1-tert-butoxycarbonyl-3-naphthalen-2-ylmethoxy-piperidin-4-yl)-phenyl]-acetic acid in 5 ml of methylene chloride was treated with 1 ml of ethereal diazomethane solution at room temperature and stirred for a further 2 hours. The reaction solution was evaporated under reduced pressure and the tert-butyl (3RS,4RS)-4-(4-methoxycarbonylmethyl-phenyl)-3-(naphthalen-2-ylmethoxy)-piperidine-1-carboxylate, R_{f}: 0.5 (hexane:ethyl acetate=2:1), obtained in quantitative yield, was used in the following step without further purification and characterization.

(b) A solution of 102 mg (0.21 mmol) of tert-butyl (3RS,4RS)-4-(4-methoxycarbonylmethyl-phenyl)-3-(naphthalen-2-ylmethoxy)-piperidine-1-carboxylate in 0.5 ml of hydrazine hydrate was heated to 120° C. for 18 hours. For the working-up, the reaction mixture was cooled to room temperature, treated with 3 ml of ice-water and extracted twice with 5 ml of methylene chloride each time. The combined organic phases were dried over sodium sulfate and evaporated under reduced pressure. There were obtained 63 mg (63% of theory) of tert-butyl (3RS,4RS)-4-(4-hydrazinocarbonylmethyl-phenyl)-3-(naphthalen-2-ylmethoxy)-piperidine-1-carboxylate as a colorless foam; R_{f}: 0.22 (SiO2, methylene chloride:methanol:ammonia=95:5:0.1).

(c) A mixture of 60 mg (0.12 mmol) of tert-butyl (3RS,4RS)-4-(4-hydrazinocarbonylmethyl-phenyl)-3-(naphthalen-2-ylmethoxy)-piperidine-1-carboxylate and 27.7 gl (0.12 mmol) of triethyl orthobenzoate in 5 ml of ethanol was boiled under reflux for 18 hours. After cooling the mixture was evaporated under reduced pressure and the residue was chromatographed on silica gel using a 2:1 mixture of hexane and ethyl acetate as the eluent. There were obtained 30 mg (44% of theory) or tert-butyl (3RS,4RS)-3-(naphthalen-2-ylmethoxy)-4-[4-(5-phenyl-[1,3,4]oxadiazol-2-ylmethyl)-phenyl]-piperidine-1-carboxylate as a colorless foam; MS: 575 (M+H)⁺.

(d) In an analogous manner to that described in Example 22(l), starting from tert-butyl (3RS,4RS)-3-(naphthalen-2-ylmethoxy)-4-[4-(5-phenyl-[1,3,4]oxadiazol-2-ylmethyl)-phenyl]-piperidine-1-carboxylate by cleavage of the BOC group using trifluoroacetic acid in methylene chloride there was obtained (3RS,4RS)-3-naphthalen-2-ylmethoxy-4-[4-(5-phenyl-[1,3,4)oxadiazol-2-ylmethyl)-phenyl]-piperidine trifluoroacetate as a white powder; MS: 476 (M+H)⁺.

### Example 40

(a) 70 mg (0.51 mmol) of phenylmagnesium chloride were added using a syringe to a solution of 80 mg (0.17 mmol) of tert-butyl (3RS,4RS)-4-[4-(2-cyano-ethyl)-phenyl]-3-naphthalen-2-ylmethoxy-piperidine-1-carboxylate (Example 35) in 10 ml of toluene. The reaction mixture was boiled under reflux for 3 hours, thereafter cooled to room temperature and hydrolyzed with 4 ml of 1N hydrochloric acid. The mixture was subsequently stirred at 80° C. for 1 hour, then cooled to room temperature and extracted twice with ethyl acetate. The combined organic phases were dried over sodium sulfate and evaporated under reduced pressure. The residue was chromatographed on silica gel using a 90:10:0.1 mixture of methylene chloride, methanol and ammonia as the eluent. The resulting mixture of (3RS,4RS)-3-[4-(3-naphthalen-2-ylmethoxy-piperidin-4-yl)-phenyl]-1-phenylpropan-1-one and (3RS,4RS)-3-{4-[3-(naphthalen-2-ylmethoxy)-piperidin-4-yl)-phenyl}-propionic acid was converted into the corresponding BOC derivative in an analogous manner to that described in Example 1 (f) and subsequently chromatographed on silica gel using a 2:1 mikxture of hexane and ethyl acetate as the eluent. There were obtained 13 mg (14% of theory) of tert-butyl (3RS,4RS)-3-(naphthalen-2-ylmethoxy)-4-[4-(3-oxo-3-phenyl-propyl)-phenyl]-piperidine-1-carboxylate as a colorless foam; R.sub.f: 0.38 (hexane:=2:1).

(b) In an analogous manner to that described in Example 22(l), starting from tert-butyl (3RS,4RS)-3-(naphthalen-2-ylmethoxy)-4-[4-(3-oxo-3-phenyl-propyl)-phenyl-piperidine-1-carboxylate by cleavage of the BOC group using trifluoroacetic acid in methylene chloride there was obtained (3RS,4RS)-3-[4-(3-naphthalen-2-ylmethoxy-piperidin-4-yl)-phenyl]-1-phenyl-propan-1-one trifluoroacetate as a white powder; MS: 450 (M+H)⁺.

### Example 41

(a) A solution of 60 mg (0.13 mmol) of tert-butyl (3RS,4RS)-4-(3-methoxycarbonyl-phenyl)-3-naphthalen-2-ylmethoxy-piperidine-1-carboxylate (Example 26(e)] and 0.26 ml (0.26 mmol) of 1N sodium hydroxide solution in 2 ml of methanol was stirred at 30° C. for 18 hours. For the working-up, the reaction mixture was neutralized with 1N hydrochloric acid and extracted twice with 10 ml of methylene chloride each time. The organic phases were combined, dried over sodium sulfate and evaporated under reduced pressure. The residue was crystallized from a mixture of hexane and diethyl ether. There were obtained 45 mg (75% of theory) of (3RS,4RS)-3-(1-tert-butoxycarbonyl-3-naphthalen-2-ylmethoxy-piperidin-4-yl)-benzoic acid as colorless crystals; MS: 462 (M+H)⁺.

(b) In an analogous manner to that described in Example 36(b), by condensing (3RS,4RS)-3-(1-tert-butoxycarbonyl-3-naphthalen-2-ylmethoxy-piperidin-4-yl)-benzoic acid with benzylamine using 1,1-carbonyldiimidazole as the condensation agent there was obtained tert-butyl (3RS,4RS)-4-(3-benzylcarbamoyl-phenyl)-3-(naphthalen-2-ylmethoxy)-piperidine-1-carboxylate as a colorless oil; MS: 551 (M+H)⁺.

(c) In an analogous manner to that described in Example 22(l), by cleavage of the BOC group from tert-butyl (3RS,4RS)-4-(3-benzylcarbamoyl-phenyl)-3-(naphthalen-2-ylmethoxy)-piperidine-1-carboxylate there was obtained (3RS,4RS)-N-benzyl-3-(3-naphthalen-2-ylmethoxy-piperidin-4-yl)-benzamide hydrochloride as a white powder; MS: 451 (M+H)⁺.

### Example 42

The following compounds were obtained in an analogous manner to that described in Example 11(l) by cleavage of the BOC group using trifluoroacetic acid in methylene chloride:

1)—(3RS,4RS)-N-benzyl-4-[3-(naphthalen-2-ylmethoxy)-piperidin-4-yl]-benzamide as a white powder, MS: 453 (M+H)⁺, from tert-butyl (3RS,4RS)-4-(4-benzylcarbamoyl-phenyl)-3-(naphthalen-2-ylmethoxy)-piperidine-1-carboxylate;

2)—(3RS,4RS)-4-[3-(naphthalen-2-ylmethoxy)-piperidin-4-yl]-N-(3-oxo-3-phenyl-propyl)-benzamide trifluoroacetate as a white powder, MS: 493 (M+H)⁺, from tert-butyl (3RS,4RS)-3-(naphthalen-2-ylmethoxy)-4-[4-(3-oxo-3-phenyl-propylcarbamoyl)-phenyl]-piperidine-1-carboxylate;

3)—(3RS,4RS)-4-[3-(naphthalen-2-ylmethoxy)-piperidin-4-yl]-N-(2-oxo-2-phenyl-ethyl)-benzamide trifluoroacetate as a white powder, MS: 479 (M+H)⁺, from tert-butyl (3RS,4RS)-3-(naphthalen-2-ylmethoxy)-4-[4-(2-oxo-2-phenyl-ethylcarbamoyl)-phenyl]-piperidine-1-carboxamide.

The BOC derivatives used as the starting materials were obtained as follows:

(a) In an analogous manner to that described in Example 41(b), by the alkaline saponification of tert-butyl (3RS,4RS)-4-(4-methoxy-carbonyl-phenyl)-3-naphthalen-2-ylmethoxy-piperidine-1-carboxylate there was obtained (3RS,4RS)-4-(1-tert-butoxy-carbonyl-3-naphthalen-2-ylmethoxy-piperidin-4-yl)-benzoic acid as a colorless solid; MS: 461 (M)⁺.

(b) In an analogous manner to that described in Example 36(b), by condensing (3 RS,4RS)-4-(1-tert-butoxycarbonyl-3-naphthalen-2-ylmethoxy-piperidin-4-yl)-benzoic acid with benzylamine there was obtained tert-butyl (3RS,4RS)-4-(4-benzylcarbamoyl-phenyl)-3-(naphthalen-2-ylmethoxy)-piperidine-1-carboxylate as a colorless foam; MS: 551 (M+H)⁺.

(c) In an analogous manner to that described in Example 36(b), by condensing (3RS,4RS)-4-(1-tert-butoxycarbonyl-3-naphthalen-2-ylmethoxy-piperidin-4-yl)-benzoic acid with 3-amino-1-phenyl-propan-1-one there was obtained tert-butyl (3RS,4RS)-3-(naphthalen-2-ylmethoxy)-4-[4-(3-oxo-3-phenyl-propylcarbamoyl)-phenyl]-piperidine-1-carboxylate as a colorless foam; MS: 593 (M+H)⁺.

(d) In an analogous manner to that described in Example 36(b), by condensing (3RS,4RS)-4-(1-tert-butoxycarbonyl-3-naphthalen-2-ylmethoxy-piperidin-4-yl)-benzoic acid with 2-amino-1-phenyl-ethanone there was obtained tert-butyl (3RS,4RS)-3-(naphthalen-2-ylmethoxy)-4-[4-(2-oxo-2-phenyl-ethylcarbamoyl)-phenyl]-piperidine-1-carboxylate as a colorless foam; MS: 579 (M+H)⁺.

### Example 43

(a) A solution of 1.0 g (3 mmol) of tert-butyl (3RS,4RS)-3-hydroxy-4-(4-methoxycarbonyl-phenyl)-piperidine-1-carboxylate in 100 ml of methanol was treated with 100 mg of rhodium (5%) on aluminium oxide and hydrogenated for 5 hours at 50° C. under 10 bar of hydrogen. Thereafter, the reaction mixture was filtered over 30 g of Dicalit, the filter aid was rinsed with 200 ml of methanol and the solution obtained was evaporated under reduced pressure. There was obtained in quantitative yield a 2:1 or 1:2 mixture of tert-butyl (3RS,4RS)-3-hydroxy-4-(trans- and -(cis-4-methoxycarbonyl-cyclohexyl)-piperidine-4-carboxylate as a colorless solid; MS: 342 (M+H)⁺.

The following procedure was carried out in an analogous manner to that described in Example 22(e) and (h)-(l):

(b) From a 2:1 or 1:2 mixture of tert-butyl (3RS,4RS)-3-hydroxy-4-(trans- and -(cis-4-methoxycarbonyl-cyclohexyl)-piperidin-4-carboxylate by reduction using lithium borohydride there was obtained a 2:1 or 1:2 mixture of tert-butyl (3RS,4RS)-cis- and -trans-3-hydroxy-4-(4-hydroxymethyl-cyclohexyl)-piperidine-1-carboxylate as a colorless foam; MS: 314 (M+H)⁺.

(c) From a 2:1 or 1:2 mixture of tert-butyl (3RS,4RS)-cis- and -trans-3-hydroxy-4-(4-hydroxymethyl-cyclohexyl)-piperidine-1-carboxylate there was obtained by introduction of the trityl group a 2:1 or 1:2 mixture of tert-butyl (3RS,4RS)-cis- and -trans-3-hydroxy-4-(4-trityloxymethyl-cyclohexyl)-piperidine-1-carboxylate as a colorless foam; MS: 573 (M+H)⁺.

(d) Alkylation of a 2:1 or 1:2 mixture of tert-butyl (3RS,4RS)-cis- and -trans-3-hydroxy-4-(4-trityloxymethyl-cyclohexyl)-piperidine-1-carboxylate with 2-bromomethylnaphthalene gave a 2:1 or 1:2 mixture of tert-butyl (3RS,4RS)-cis- and -trans-3-naphthalen-2-ylmethoxy-4-(4-trityloxymethyl-cyclohexyl)-piperidine-1-carboxylate as a colorless foam: MS: 713 (M+NH₄)⁺.

(e) From a 2:1 or 1:2 mixture of tert-butyl (3RS,4RS)-cis- and -trans-3-naphthalen-2-ylmethoxy-4-(4-trityloxymethyl-cyclohexyl)-piperidine-1-carboxylate by cleavage of the trityl group using hydrogen chloride in methanol there was obtained a 2:1 or 1:2 mixture of tert-butyl (3RS,4RS)-cis- and -trans-3-naphthalen-2-ylmethoxy-4-(4-hydroxymethyl-cyclohexyl)-piperidine-1-carboxylate as a colorless foam; MS: 453 (M+H)⁺.

(f) Acylation of a 2:1 or 1:2 mixture of tert-butyl (3RS,4RS)-cis- and -trans-3-naphthalen-2-ylmethoxy-4-(4-hydroxymethyl-cyclohexyl)-piperidine-1-carboxylate with benzoyl chloride yielded a 2:1 or 1:2 mixture of tert-butyl (3RS,4RS)-cis- and -trans-4-(4-benzoyloxymethyl-cyclohexyl)-3-naphthalen-2-ylmethoxy-piperidine-1-carboxylate as a colorless oil; MS: 558 (M+H)⁺.

(g) From a 2:1 or 1:2 mixture of tert-butyl (3RS,4RS)-cis- and -trans-4-(4-benzoyloxymethyl-cyclohexyl)-3-naphthalen-2-ylmethoxy-piperidine-1-carboxylate by cleavage of the BOC group using hydrogen chloride in methanol there was obtained a 2:1 or 1:2 mixture of (3RS,4RS)-cis- and -trans 4-(3-naphthalen-2-ylmethoxy-piperidin-4-yl)-cyclohexylmethyl benzoate as a colorless foam; MS: 458 (M+H)⁺.

### Example 44

The following procedure was carried out in an analogous manner to that described in Example 22(a)-(c):

(a) 1-Benzyl-4-(4-methoxy-phenyl)-piperidin-4-ol was obtained as a colorless solid, MS: 298 (M+H)⁺, from 1-benzyl-4-piperidone and 4-iodoanisole.

(b) 1-Benzyl-4-(4-methoxy-phenyl)-1,2,3,6-tetrahydro-pyridine was obtained as a beige solid, MS: 280 (M+H)⁺, from 1-benzyl-4-(4-methoxy-phenyl)-piperidin-4-ol by elimination.

(c) Hydroboration of 1-benzyl-4-(4-methoxy-phenyl)-1,2,3,6-tetrahydro-pyridine gave (3RS,4RS)-1-benzyl-4-(4-methoxy-phenyl)-piperidin-3-ol as colorless crystals; MS: 297 (M)⁺.

(d) 49.6 ml (49.6 mmol, 2 eq.) of an approximately 1M boron tribromide solution in methylene chloride was added dropwise at 3-7° C. within 10 minutes to a solution of 7.38 g (24.82 mmol) of (3RS,4RS)-1-benzyl-4-(4-methoxy-phenyl)-piperidin-3-ol in 248 ml of methylene chloride. This suspension was stirred at room temperature for 3 hours. Subsequently, the reaction mixture was poured into 750 ml of an ice/water mixture, brought to pH 8 with 2N sodium hydroxide solution and extracted three times with 500 ml of methylene chloride each time. The organic phases were washed with a small amount of water, dried over magnesium sulfate, evaporated under reduced pressure and dried in a high vacuum. This yielded 6.42 g of (3RS,4RS)-1-benzyl-4-(4-hydroxy-phenyl)-piperidin-3-ol in the form of a white foam; MS: 283 (M)⁺.

(e) A solution of 3.0 g, (10.6 mmol) of (3RS,4RS)-1-benzyl-4-(4-hydroxy-phenyl)-piperidin-3-ol in 75 ml of ethyl methyl ketone was treated in succession with 10.84 g (42.4 mmol, 4.8 eq) of 2-(2-iodo-ethoxy)-tetrahydro-pyran and 5.25 g (53 mmol, 5 eq) of potassium carbonate. This mixture was stirred at 95° C. for 25 hours. Subsequently, it was concentrated to a few millilitres, poured into 200 ml of an ice/water mixture and extracted three times with 300 ml of methylene chloride each time. The combined organic phases were washed once with a small amount of water, dried over magnesium sulfate, evaporated under reduced pressure and dried in a high vacuum. The thus-obtained crude product (11.81 g) was separated on silica gel using a 99:1 mixture of methylene chloride and methanol as the eluent and yielded 3.2 g (73% of theory) of a mixture of (3RS,4RS)-1-benzyl-4-[4-[2-[(RS)- and -[(SR)-tetrahydro-pyran-2-yloxy]-ethoxy]-phenyl]-piperidin-3-ol as a colorless oil; R.sub.f: 0.55 (SiO₂, methylene chloride:methanol=9:1).

(f) In an analogous manner to that described in Example 22(i), by alkylating a mixture of (3RS,4RS)-1-benzyl-4-[4-[2-[(RS)- and -[(SR)-tetrahydro-pyran-2-yloxy]-ethoxy]-phenyl]-piperidin-3-ol with 2-bromomethylnaphthalene there was obtained a mixture of (3RS,4RS)-1-benzyl-3-(naphthalen-2-ylmethoxy)-4-[4-[2-[(RS)- and -[(SR)-tetrahydro-pyran-2-yloxy]-ethoxy]-phenyl]-piperidine as a colorless oil; R_{f}: 0.46 (SiO₂, methylene chloride:=1:1).

(g) In an analogous manner to that described Example 25(a), by cleavage of the benzyl group using 2,2,2-trichloroethyl chloroformate and potassium carbonate there was obtained a mixture of 2,2,2-trichloro-ethyl (3RS,4RS)-3-(naphthalen-2-ylmethoxy)-4-[4-[2-[(RS)- and -[(SR)-tetrahydro-pyran-2-yloxy]-ethoxy]-phenyl]-piperidine-1-carboxylate as a colorless oil; MS: 653.3, 655 (M+H)⁺.

(h) A solution of 500 mg (0.785 mmol) of a mixture of 2,2,2-trichloro-ethyl (3RS,4RS)-3-(naphthalen-2-ylmethoxy)-4-[4-[2-[(RS)- and -[(SR)-tetrahydro-pyran-2-yloxy]-ethoxy]-phenyl]-piperidine-1-carboxylate in 10 ml of methanol was treated with 1 ml of water and 1.120 g (5.888 mmol) of p-toluenesulfonic acid monohydrate. This suspension was stirred at room temperature for 1.5 hours, then concentrated to half of the volume under reduced pressure and extracted four times with methylene chloride against water. The organic phases were each washed once with saturated sodium hydrogen carbonate solution and saturated sodium chloride solution, dried over magnesium sulfate, evaporated under reduced pressure and dried in a high vacuum. The crude product (465 mg) was separated on silica gel using a 9:1 mixture of methylene chloride and methanol as the eluent. This yielded 344 mg (79% of theory) of 2,2,2-trichloroethyl (3RS,4RS)-4-[4-(2-hydroxy-ethoxy)-phenyl]-3-(naphthalen-2-ylmethoxy)-piperidine-1-carboxylate as a colorless, amorphous powder; MS: 569.3, 571 (M+NH₄)⁺.

(i) In an analogous manner to that described in Example 24(m), from 2,2,2-trichloroethyl (3RS,4RS)-4-[4-(2-hydroxy-ethoxy)-phenyl]-3-(naphthalen-2-ylmethoxy)-piperidine-1-carboxylate and pyridine-2-carbonyl azide there was obtained 2,2,2-trichloro-ethyl (3RS,4RS)-3-(naphthalen-2-ylmethoxy)-4-[4-(2-pyridin-2-ylcarbamoyloxy-ethoxy]-phenyl]-piperidine-1-carboxylate as a colorless oil; MS: 672.2, 674 (M+H)⁺.

(j) In an analogous manner to that described in Example 25(b), by treating 2,2,2-trichloroethyl (3RS,4RS)-3-(naphthalen-2-ylmethoxy)-4-[4-(2-pyridin-2-ylcarbamoyloxy-ethoxy]-phenyl]-piperidine-1-carboxylate with zinc in glacial acetic acid there was obtained (3RS,4RS)-2-[4-[3-(naphthalen-2-ylmethoxy)-piperidin-4-yl]-phenoxy]-ethyl pyridine-2-yl-carbamate as a colorless solid; MS: 498 (M+H)⁺.

### Example 45

The following compounds were obtained in an analogous manner to that described in Example 25(b):

1)—(3RS,4RS)-2-[4-[3-(Naphthalen-2-ylmethoxy)-piperidin-4-yl]-phenoxy]-ethyl carbamate as a colorless oil, MS: 421 (M+H)⁺, from 2,2,2-trichloro-ethyl (3RS,4RS)-4-[4-(2-carbamoyloxy-ethoxy)-phenyl]-3-(naphthalen-2-ylmethoxy)-piperidine-1-carboxylate;

2)—(3RS,4RS)-2-14-[3-(naphthalen-2-ylmethoxy)-piperidin-4-yl]-phenoxy]-ethyl morpholine-4-carboxylate as a colorless oil, MS: 491 (M+H)⁺, from 2,2,2-trichloro-ethyl (3RS,4RS)-4-[4-[2-(morpholin-4-ylcarbonyloxy)-ethoxy]-phenyl]-3-(naphthalen-2-ylmethoxy)-piperidine-1-carboxylate;

3)—(3RS,4RS)-4-(4-methoxy-phenyl)-3-(naphthalen-2-ylmethoxy)-piperidine as a colorless oil, MS: 348 (M+H)⁺, from 2,2,2-trichloro-ethyl (3RS,4RS)-4-(4-methoxy-phenyl)-3 (naphthalen-2-ylmethoxy)-piperidine-1-carboxylate;

4)—(3RS,4RS)-2-[4-[3-(naphthalen-2-ylmethoxy)-piperidin-4-yl]-phenoxy]-ethanol as a colorless, amorphous solid, MS: 378 (M+H)⁺, from a mixture of 2,2,2-trichloro-ethyl (3RS,4RS)-3-(naphthalen-2-ylmethoxy)-4-[4-[2-[(RS)- and -[(SR)-tetrahydropyran-2-yloxy]-ethoxy]-phenyl]-piperidine-1-carboxylate with simultaneous cleavage of the THP group.

The derivatives used as the starting materials were obtained as follows:

(a) In an analogous manner to that described in Example 24 (m), from 2,2,2-trichloroethyl (3RS,4RS)-4-[4-(2-hydroxy-ethoxy)-phenyl]-3-(naphthalen-2-ylmethoxy)-piperidine-1-carboxylate and sodium isocyanate there was obtained 2,2,2-trichloro-ethyl (3RS,4RS)-4-(4-(2-carbamoyloxy-ethoxy)-phenyl]-3-(naphthalen-2-ylmethoxy)-piperidine-1-carboxylate as a colorless oil; R_{f}: 0.28 (SiO₂, methylene chloride:acetone=1:1).

(b) A solution of 250 mg (0.56 mmol) of 2,2,2-trichloroethyl (3RS,4RS)-4-[4-(2-hydroxy-ethoxy)-phenyl]-3-(naphthalen-2-ylmethoxy)-piperidine-1-carboxylate in 20 ml of toluene was treated in succession with 250 ml (2.8 mmol, 5.0 eq.) of morpholine-4-carbonyl chloride, 375 mg (3.08 mmol, 5.5 eq.) of 4-dimethylaminopyridine and 20 gl (0.075 mmol, 0.13 eq.) of dibutyltin diacetate. This mixture was boiled under reflux for 64 hours. In the course of the reaction a further 125 ml (1.40 mmol, 5.0 eq.) of morpholine-4-carbonyl chloride and 188 mg (1.56 mmol, 2.3 eq.) of 4-dimethylaminopyridine were added and the mixture was boiled under reflux for a further 24 hours. The reaction mixture, cooled to room temperature, was poured into 100 ml of an ice/water mixture, stirred for 5 minutes and extracted three times with methylene chloride. The organic phases were each washed once with water and saturated sodium chloride solution, dried over magnesium sulfate, evaporated under reduced pressure and dried in a high vacuum. The crude product (546 mg) was separated on silica gel using a 9:1 mixture of hexane and acetone as the eluent. There were obtained 42 mg (14% of theory) of 2,2,2-trichloro-ethyl (3RS,4RS)-4-[4-[2-(morpholin-4-ylcarbonyloxy)-ethoxy]-phenyl]-3-(naphthalen-2-ylmethoxy)-piperidine-1-carboxylate as a colorless oil; MS: 665.3, 667 (M+H)⁺.

(c) In an analogous manner to that described in Example 12(b), by alkylating (3RS,4RS)-1-benzyl-4-(4-methoxy-phenyl)-piperidin-3-ol [Example 44 (c)] with 2-bromomethylnaphthalene there was obtained (3RS,4RS)-1-benzyl-4-(4-methoxy-phenyl)-3-(naphthalen-2-ylmethoxy)-piperidine as a beige colored solid; MS: 437 (M)⁺. Subsequent reaction with 2,2,2-trichloroethyl chloroformate gave 2,2,2-trichloro-ethyl (3RS,4RS)-4-(4-methoxy-phenyl)-3-(naphthalen-2-ylmethoxy)-piperidine-1-carboxylate as a yellowish oil; MS: 539, 541 (M+NH₄)⁺.

### Example 46

(a) In an analogous manner to that described in Example 2(e), by hydrogenolytic cleavage of the benzyl group from (3RS,4RS)-1-benzyl-4-(4-hydroxy-phenyl)-piperidin-3-ol [Example 44(d)] there was obtained (3RS,4RS)-4-(4-hydroxy-phenyl)-piperidin-3-ol as a colorless solid; MS: 194 (M+H)⁺.

b) In an analogous manner to that described in Example 22(g), from (3RS,4RS)-4-(4-hydroxy-phenyl)-piperidin-3-ol by introduction of the BOC group there was obtained tert-butyl (3RS,4RS)-3-hydroxy-4-(4-hydroxy-phenyl)-piperidine-1-carboxylate as a colorless foam; MS: 237 (M-C₄H₈)⁺.

(c) A mixture of 4.5 g (15.3 mmol) of tert-butyl (3RS,4RS)-3-hydroxy-4-(4-hydroxy-phenyl)-piperidine-1-carboxylate, 20.8 g (91.8 mmol, 6.0 eq.) of 2-(3-chloropropyl)-2-phenyl-[1,3]dioxolane [J. Med. Chem. 34, 12 (1991)], 14.6 g (105.5 mmol, 6.9 eq.) of potassium carbonate and 2.0 g (0.012 mmol, 0.078 eq.) of potassium iodide in 50 ml of methyl ethyl ketone were stirred in a sealed, pressure-tight vessel at a bath temperature of 100° C. for 60 hours. The reaction mixture was poured into an ice/water mixture and extracted three times with ethyl acetate. The organic phases were each washed once with water and saturated sodium chloride solution, dried over magnesium sulfate, concentrated under reduced pressure and dried in a high vacuum. The yellow oil (25.72 g) was separated on silica gel using an elution gradient of 4:1 to 1:1 of a mixture of hexane and ethyl acetate as the eluent. This yielded 5.34 g (72% of theory) of white, crystalline tert-butyl (3RS,4RS)-3-hydroxy-4-[4-[3-(2-phenyl-[1,3]dioxolan-2-yl)-propoxy]-phenyl]-piperidine-1-carboxylate; MS: 484 (M+H)⁺.

(d) In an analogous manner to that described in Example 22(i), by alkylating tert-butyl (3RS,4RS)-3-hydroxy-4-[4-[3-(2-phenyl-[1,3]dioxolan-2-yl)-propoxy]-phenyl]-piperidine-1-carboxylate with 2-bromomethylnaphthalene there was obtained tert-butyl (3RS,4RS)-3-(naphthalen-2-ylmethoxy)-4-[4-[3-(2-phenyl-[1,3]dioxolan-2-yl)-propoxy)-phenyl]-piperidine-1-carboxylate as white crystals; MS: 624 (M+H)⁺.

(e) A solution of 193 mg (0.31 mmol) of tert-butyl (3RS,4RS)-3-(naphthalen-2-ylmethoxy)-4-[4-[3-(2-phenyl-[1,3]dioxolan-2-yl)-propoxy]-phenyl]-piperidine-1-carboxylate in 3 ml of tetrahydrofuran and 3 ml of 2N hydrochloric acid was stirred at room temperature for 12 hours and at 50° C. for 24 hours. Subsequently, the reaction mixture was poured into a 1:1 mixture of water and saturated sodium hydrogen carbonate solution and extracted three times with ethyl acetate. The organic phases were each washed once with water and saturated sodium chloride solution, dried over magnesium sulfate, concentrated under reduced pressure and dried in a high vacuum. The colorless oil (165 mg) was separated on silica gel using a 9:1 mixture of methylene chloride and methanol (extr. against 5 vol. % conc. NH₄ OH). This yielded 127.4 mg of (3RS,4RS)-4-[4-(3-naphthalen-2-ylmethoxy-piperidin-4-yl)-phenoxy]-1-phenyl-butan-1-one in the form of a colorless oil; MS: 502 (M+Na)⁺.

### Example 47

The following compounds were obtained in an analogous manner to that described in Example 46(d)-(e) and, respectively, 3(c)-(e):

1)—From tert-butyl (3RS,4RS)-3-hydroxy-4-[4-[3-(2-phenyl-[1,3]dioxolan-2-yl)-propoxy]-phenyl]-piperidine-1-carboxylate by alkylation with 5-bromomethyl-benzo[b]thiophene there was obtained tert-butyl (3RS,4RS)-3-(benzo[b]thiophen-5-ylmethoxy)-4-{4-[3-(2-phenyl-[1,3]dioxolan-2-yl)-propoxy]-phenyl}-piperidine-1-carboxylate, MS: 630 (M+H)⁺, as a light yellow resin. Subsequent cleavage of the BOC and acetal groups yielded (3RS,4RS)-4-[4-[3-(benzo[b]thiophen-5-ylmethoxy)-piperidin-4-yl]-phenoxy]-1-phenyl-butan-1-one as a colorless resin; MS: 486 (M+H)⁺.

2)—From tert-butyl (3RS,4RS)-3-hydroxy-4-[4-[3-(2-phenyl-[1,3]dioxolan-2-yl)-propoxy]-phenyl]-piperidine-1-carboxylate by alkylation with 5-(chloromethyl)indane there was obtained tert-butyl (3RS,4RS)-3-(indan-5-ylmethoxy)-4-{4-[3-(2-phenyl-[1,3]dioxolan-2-yl)-propoxy]-phenyl}-piperidine-1-carboxylate, MS: 614 (M+H)⁺, as a light yellow resin. Subsequent cleavage of the BOC and acetyl groups yielded (3RS,4RS)-4-[4-[3-indan-5-ylmethoxy)-piperidin-4-yl]-phenoxy]-1-phenyl-butan-1-one as a colorless resin; MS: 470 (M+H)⁺.

3)—From tert-butyl (3RS,4RS)-3-hydroxy-4-[4-[3-(2-phenyl-[1,3]dioxolan-2-yl)-propoxy]-phenyl)-piperidine-1-carboxylate by alkylation with 3-chloromethyl-1-(2-trimethylsilanyl-ethoxy-methyl)-naphthalene there was obtained tert-butyl (3RS,4RS)-4-[4-[3-(2-phenyl-[1,3]dioxolan-2-yl)-propoxy]-phenyl]-3-[4-(2-trimethylsilanyl-ethoxymethoxy)-naphthalen-2-ylmethoxy]-piperidine-1-carboxylate, MS: 770 (M+H)⁺, as a colorless resin. Subsequent cleavage of the BOC group and of the two acetal groups yielded (3RS,4RS)-4-[4-[3-(4-hydroxy-naphthalen-2-ylmethoxy)-piperidin-4-yl]-phenoxy]-1-phenyl-butan-1-one as a colorless resin; R_{f}: 0.17 (SiO₂, methylene chloride:methanol=9:1, extracted against 5% ammonia).

4)—From tert-butyl (3RS,4RS)-3-hydroxy-4-(4-hydroxy-phenyl)-piperidine-1-carboxylate by alkylation with 1-bromo-4-(2-bromoethyl)-benzene analogously to Example 46(c) there was obtained tert-butyl (3RS,4RS)-4-[4-[2-(4-bromo-phenoxy)-ethoxy]-phenyl]-3-hydroxy-piperidine-1-carboxylate as a colorless resin. Further alkylation with 2-bromomethylnaphthalene gave tert-butyl (3RS,4RS)-4-[4-[2-(4-bromo-phenoxy)-ethoxy]-phenyl]-3-(naphthalen-2-ylmethoxy)-piperidine-1-carboxylate, from which by cleavage of the BOC group there was obtained (3RS,4RS)-4-[4-[2-(4-bromo-phenoxy)-ethoxy]-phenyl]-3-(naphthalen-2-ylmethoxy)-piperidine as a colorless resin; MS: 532, 534M+H)⁺.

5)—From tert-butyl (3RS,4RS)-3-hydroxy-4-(4-hydroxy-phenyl)-piperidine-1-carboxylate by alkylation with 2-bromomethyl-5-phenyl-[1,3,4]oxadiazole analogously to Example 44(e) there was obtained tert-butyl (3RS,4RS)-3-hydroxy-4-[4-(5-phenyl-[1,3,4]oxadiazol-2-ylmethoxy)-phenyl)-piperidine-1-carboxylate as a yellowish, amorphous solid. Further alkylation with 2-bromomethylnaphthalene gave tert-butyl (3RS,4RS)-3-(naphthalen-2-ylmethoxy)-4-[4-(5-phenyl-[1,3,4]oxadiazol-2-ylmethoxy)-phenyl]-piperidine-1-carboxylate, from which by cleavage of the BOC group there was obtained (3RS,4RS)-3-(naphthalen-2-ylmethoxy)-4-[4-(5-phenyl-[1,3,4]oxadiazol-2-ylmethoxy)-phenyl]piperidine as a colorless solid; MS: 492 (M+H)⁺.

6)—From (3RS,4RS)-3-hydroxy-4-(4-hydroxy-phenyl)-piperidine-1-carboxylate by alkylation with β-bromophenethol analogously to Example 46(c) there was obtained tert-butyl (3RS,4RS)-3-hydroxy-4-[4-(2-phenoxy-ethoxy)-phenyl]-piperidine-1-carboxylate; MS: 414 (M+H)⁺. Further alkylation with 2-bromomethylnaphthalene gave tert-butyl (3RS,4RS)-3-(naphthalen-2-ylmethoxy)-4-[4-(2-phenoxy-ethoxy)-phenyl]-piperidine-1-carboxylate, MS: 554 (M+H)⁺, from which by cleavage of the BOC group there was obtained (3RS,4RS)-3-(naphthalen-2-ylmethoxy)-4-[4-(2-phenoxy-ethoxy)-phenyl]-piperidine; MS: 454 (M+H)⁺.

7)—From tert-butyl (3RS,4RS)-3-hydroxy-4-[4-(2-phenoxy-ethoxy)-phenyl]-piperidine-1-carboxylate by alkylation with 2-chloromethyl-O-SEM there was obtained tert-butyl (3RS,4RS)-4-[4-(2-phenoxy-ethoxy)-phenyl]-3-[4-(2-trimethylsilanyl-ethoxy-methoxy)-naphthalen-2-ylmethoxy]-piperidine-1-carboxylate; MS: 700 (M+H)⁺. Subsequent cleavage of the BOC and acetal groups yielded (3RS,4RS)-3-{4-[4-(2-phenoxy-ethoxy)-phenyl]-piperidin-3-yloxymethyl}-naphthalen-1-ol; MS: 470 (M+H)⁺.

### Example 48

A solution of 18.7 mg (0.494 mmol, 6.7 eq.) of sodium borohydride in 0.35 ml of water was added using a syringe to a solution of 44 mg (0.074 mmol) of (3RS,4RS)-4-[4-[3-(4-hydroxy-naphthalen-2-ylmethoxy)-piperidin-4-yl)-phenoxy]-1-phenyl-butan-1-one in 1.5 ml of dioxan. The mixture was stirred at room temperature for 1.5 hours. Subsequently, the reaction solution was treated with the same amount by volume of ice-water and the mixture was brought to pH 1 with 2N hydrochloric acid. After stirring at room temperature for 5-10 minutes the mixture was s adjusted to pH 9 with concentrated ammonia and the aqueous solution was extracted three times with one equivalent by volume of methylene chloride each time. The combined organic phases were dried over magnesium sulfate, concentrated at 45° C. under reduced pressure and dried in a high vacuum. The brownish solid (40.1 mg) was separated on silica gel using a 9:1 mixture of methylene chloride and methanol (extr. against 5 vol. % conc. NH₃ aq.) as the eluent. This yielded 13 mg (35% of theory) of (3RS,4RS)-3-[4-[4-(4-hydroxy-4-phenyl-butoxy)-phenyl]-piperidin-4-yloxymethyl]-naphthalen-1-ol (configuration unknown in the butanol part) as a colorless oil; MS: 498 (M+H)⁺.

### Example 49

The following alcohols were obtained in an analogous manner to that described in Example 48 by reduction of the ketones:

1)—The 2:1 or 1:2 mixture of (RS)- and (SR)-4-[4-[(3RS,4RS)-3-(indan-5-ylmethoxy)-piperidin-4-yl]-phenoxy]-1-phenyl-butan-1-ol as a colorless resin, MS: 472 (M+H)⁺, from (3RS,4RS)-4-[4-[3-indan-5-ylmethoxy)-piperidin-4-yl]-phenoxy]-1-phenylbutan-1-one;

2)—The mixture of (RS)- and (SR)-4-[4-[(3RS,4RS)-3-(benzo-[b]thiopen-5-ylmethoxy)-piperidin-4-yl]-phenoxy]-1-phenyl-butan-1-ol as a colorless resin, MS: 488 (M+H))⁺, from (3RS,4RS)-4-[4-[3-(benzo[b]thiophen-5-ylmethoxy)-piperidin-4-yl]-phenoxy]-1-phenylbutan-1-one;

3)—4-[4-[(3RS,4RS)-3-naphthalen-2-ylmethoxy-piperidin-4-yl]-phenoxy]-1-phenyl-butan-1-ol (configuration unknown in the butanol part) as a colorless resin, MS: 482 (M+H)⁺, from (3RS,4RS)-4-[4-(3-naphthalen-2-ylmethoxy-piperidin-4-yl)-phenoxy]-1-phenyl-butan 1-one.

### Example 50

(a) In an analogous manner to that described in Example 22 (g), from (3RS,4RS)-4-[4-(3-naphthalen-2-ylmethoxy-piperidin-4-yl)-phenoxy]-1-phenyl-butan-1-one by introducing the BOC group there was obtained tert-butyl (3RS,4RS)-3-(naphthalen-2-ylmethoxy)-4-(4-(4-oxo-4-phenyl-butoxy)-phenyl]-piperidine-1-carboxylate as a colorless solid; MS: 580 (M+H)⁺.

(b) Reduction of tert-butyl (3RS,4RS)-3-(naphthalen-2-ylmethoxy)-4-[4-(4-oxo-4-phenyl-butoxy)-phenyl)-piperidine-1-carboxylate analogously to Example 48 yielded tert-butyl (3RS,4RS)-4-[4-(4-hydroxy-4-phenyl-butoxy)-phenyl-3-naphthalen-2-ylmethoxy-piperidine-1-carboxylate (configuration unknown in the butoxy part) as a colorless solid; MS: 582 (M+H)⁺.

(c) Alkylation of tert-butyl (3RS,4RS)-4-[4-(4-hydroxy-4-phenyl-butoxy)-phenyl]-3-naphthalen-2-ylmethoxy-piperidine-1-carboxylate with methyl iodide analogously to Example 22(i) gave tert-butyl (3RS,4RS)-4-[4-(4-methoxy-4-phenyl-butoxy)-phenyl]-3-(naphthalen-2-ylmethoxy)-piperidine-1-carboxylate (configuration unknown in the butoxy part) as a colorless oil; R_{f}: 0.61 (SiO₂, hexane:ethyl acetate=2:1).

(d) In an analogous manner to that described in Example 22(l), from tert-butyl (3RS,4RS)-4-[4-(4-methoxy-4-phenyl-butoxy)-phenyl]-3-(naphthalen-2-ylmethoxy)-piperidine-1-carboxylate by cleavage of the BOC group there was obtained (3RS,4RS)-4-[4-(4-methoxy-4-phenyl-butoxy)-phenyl]-3-(naphthalen-2-ylmethoxy)-piperidine (configuration unknown in the butoxy part) as a colorless resin; MS: 496 (M+H)⁺.

### Example 51

A solution of 30 mg (0.052 mmol) of tert-butyl (3RS,4RS)-4-[4-(4-hydroxy-4-phenyl-butoxy)-phenyl]-3-naphthalen-2-ylmethoxy-piperidine-1-carboxylate (configuration unknown in the butoxy part), 25.5 gl (0.186 mmol, 3.6 eq.) of triethylamine and 1.7 ml of methylene chloride was treated in succession with 21.6 gl (0.186 mmol, 3.6 eq.) of benzoyl chloride and 2 mg (0.016 mmol) of 4-dimethylaminopyridine. This reaction solution was stirred at room temperature for 10 hours, then poured into 5 ml of water and extracted three times with 10 ml of ethyl acetate each time. The combined organic phases were dried over magnesium sulfate, evaporated under reduced pressure and dried in a high vacuum. The light yellow resin (62 mg) was dissolved in 1 ml of methanol, again concentrated, dried in a high vacuum. Without further purification and characterization the product obtained was reacted with hydrogen chloride in methanol analogously to the procedure described in Example 22(l). The brown-yellow resin (56 mg) was separated on silica gel using a 95:5 mixture of methylene chloride and methanol (extracted against 5 vol. % conc. ammonia) as the eluent. There were obtained 15 mg (50% of theory) of 4-[4-[(3RS,4RS)-3-naphthalen-2-ylmethoxy-piperidin-4-yl]-phenoxy]-1-phenyl-butyl benzoate (configuration unknown in the butoxy part) as a colorless resin; MS: 586 (M+H)⁺.

### Example 52

A mixture of 600 mg (0.962 mmol) of tert-butyl (3RS,4RS)-3-(naphthalen-2-ylmethoxy)-4-{4-[3-(2-phenyl-[1,3]dioxolan-2-yl)-propoxy]-phenyl}-piperidine-1-carboxylate in 3.0 ml of methylene chloride and 433 mg (1.92 mmol, 2.0 eq.) of zinc bromide was stirred at room temperature for 4 hours. The reaction mixture was poured into water and extracted three times with ethyl acetate. The organic phases were washed in each case once with water and with saturated sodium chloride solution, dried over magnesium sulfate, evaporated under reduced pressure and dried in a high vacuum. The amorphous, slightly yellow crude product was separated on silica gel using a 95:5:0.1 mixture of methylene chloride, methanol and ammonia. There were obtained 355 mg (71% of theory) of (3RS,4RS)-3-(naphthalen-2-ylmethoxy)-4-[4-[3-(2-phenyl-[1,3]dioxolan-2-yl)-propoxy]-phenyl]-piperidine as a colorless resin; MS: 524 (M+H)⁺.

### Example 53

(a) In an analogous manner to that described in Example 44(e)-(f), by alkylating tert-butyl (3RS,4RS)-3-hydroxy-4-(4-hydroxy-phenyl)-piperidine-1-carboxylate with 2-(2-iodo-ethoxy)-tetrahydro-pyran there was obtained a mixture of tert-butyl (3RS,4RS)-3-hydroxy-4-[4-[2-[(RS)- and -[(SR)-tetrahydro-pyran-2-yloxy]-ethoxy]-phenyl]-piperidine-1-carboxylate as a yellow oil; R_{f}: 0.45 (SiO₂, hexane:ethyl acetate=1:1).

b) In an analogous manner to that described in Example 22(i), by alkylating the mixture of tert-butyl (3RS,4RS)-3-hydroxy-4-[4-[2-[(RS)- and -[(SR)-tetrahydro-pyran-2-yloxy]-ethoxy]-phenyl]-piperidine-1-carboxylate with 2-bromomethylnaphthalene there was obtained a mixture of tert-butyl (3RS,4RS)-3-(naphthalen-2-ylmethoxy)-4-[4-[2-[(RS)- and -[(SR)-tetrahydro-pyran-2-yloxy]-ethoxy]-phenyl]-piperidine-1-carboxylate as a colorless solid; MS: 579 (M+H)⁺.

(c) A solution of 1.99 g (3.54 mmol) of a mixture of tert-butyl (3RS,4RS)-3-(naphthalen-2-ylmethoxy)-4-[4-[2-[(RS)- and -[(SR)-tetrahydro-pyran-2-yloxy]-ethoxy]-phenyl]-piperidine-1-carboxylate in 11.3 ml of methanol was treated with 11.3 ml of 2N hydrogen chloride in methanol and the mixture was stirred at room temperature for 5 minutes. The reaction mixture was poured into 200 ml of a mixture of ice and saturated sodium hydrogen carbonate solution and extracted three times with ethyl acetate. The organic phases were washed with saturated sodium chloride solution, dried over magnesium sulfate, evaporated under reduced pressure and dried in a high vacuum. The yellow resin (1.80 g) was recrystallized from hexane. There were obtained 950 mg (56%) of tert-butyl (3RS,4RS)-4-[4-(2-hydroxy-ethoxy)-phenyl]-3-(naphthalen-2-ylmethoxy)-piperidine-1-carboxylate as a colorless solid; MS: 478 (M+H)⁺.

d) In an analogous manner to that described in Example 22(k), by acylating tert-butyl (3RS,4RS)-4-[4-(2-hydroxy-ethoxy)-phenyl]-3-(naphthalen-2-ylmethoxy)-piperidine-1-carboxylate with 4-chlorobenzoyl chloride there was obtained tert-butyl (3RS,4RS)-4-[4-[2-(4-chloro-benzoyloxy)-ethoxy]-phenyl]-3-(naphthalen-2-yl methoxy)-piperidine-1-carboxylate as a colorless oil; MS: 616 (M+H)⁺.

(e) In an analogous manner to that described in Example 22(l), from tert-butyl (3RS,4RS)-4-[4-[2-(4-chloro-benzoyloxy)-ethoxy]-phenyl)-3-(naphthalen-2-yl methoxy)-piperidine-1-carboxylate by cleavage of the BOC group there was obtained (3RS,4RS)-2-[4-(3-napthalen-2-ylmethoxy-piperidin-4-yl)-phenoxy]-ethyl 4-chloro-benzoate hydrochloride as a colorless solid; MS: 516 (M+H)⁺.

### Example 54

The following compounds were obtained in an analogous manner to that described in Example 22(l) by cleavage of the BOC group using acid:

1)—(3RS,4RS)-2-[4-[3-(Naphthalen-2-ylmethoxy)-piperidin-4-yl]-phenoxy]-ethyl 4-methoxy-benzoate hydrochloride as a colorless solid, MS: 512 (M+H)⁺, from tert-butyl (3RS,4RS)-4-[4-[2-(4-methoxy-benzoyloxy)-ethoxy]-phenyl]-3-(naphthalen-2-ylmethoxy)-piperidine-1-carboxylate;

2)—(3RS,4RS)-2-[4-(3-napthalen-2-ylmethoxy-piperidin-4-yl)-phenoxy]-ethyl 2-chloro-benzoate hydrochloride as a colorless solid, MS: 516 (M+H)⁺, from tert-butyl (3RS,4RS)-4-[4-[2-(2-chloro-benzoyloxy)-ethoxy]-phenyl]-3-(naphthalen-2-yl methoxy)-piperidine-1-carboxylate;

3)—(3RS,4RS)-2-[4-[3-(naphthalen-2-yloxy)-piperidin-4-yl]-phenoxy]-ethyl benzoate as a colorless, amorphous solid, MS: 340 (M-naphthylmethyl)⁺, from tert-butyl (3RS,4RS)-4-[4-(2-benzoyloxy-ethoxy)-phenyl]-3-(naphthalen-2-ylmethoxy)-piperidine-1-carboxylate;

4)—(3RS,4RS)-2-[4-[3-(naphthalen-2-ylmethoxy)-piperidin-4-yl]-phenoxy]-ethyl 2-benzoyloxymethyl-benzoate hydrochloride as a colorless solid, MS: 616 (M+H)⁺, from tert-butyl (3RS,4RS)-4-[4-[2-(2-benzoyloxymethyl-benzoyloxy)-ethoxy]-phenyl]-3-(naphthalen-2-ylmethoxy)-piperidine-1-carboxylate;

5)—(3RS,4RS)-3-(naphthalen-2-ylmethoxy)-4-[4-(2-phenyl-sulfanyl-ethoxy)-phenyl]-piperidine hydrochloride as a colorless solid, MS: 470 (M+H)⁺, from tert-butyl (3RS,4RS)-3-(naphthalen-2-ylmethoxy)-4-[4-(2-phenylsulfanyl-ethoxy)-phenyl]-piperidine-1-carboxylate;

6)—(3RS,4RS)-3-naphthalen-2-ylmethoxyxy-4-[4-(2-phenyl-sulfonyl-ethoxy)-phenyl]-piperidine as a yellowish foam, MS: 502 (M+H)⁺, from tert-butyl (3RS,4RS)-3-(naphthalen-2-ylmethoxy)-4-[4-(2-phenylsulfonyl-ethoxy)-phenyl]-piperidine-1-carboxylate;

7)—(3RS,4RS)-N-[2-[4-[3-(naphthalen-2-yloxy)-piperidin-4-yl]-phenoxy]-ethyl-benzamide as a colorless, amorphous solid, MS: 481 (M+H)⁺, from tert-butyl (3RS,4RS)-4-[4-(2-benzoylamino-ethoxy)-phenyl]-3-(naphthalen-2-ylmethoxy)-piperidine-1-carboxylate;

8)—(3RS,4RS)-3-(naphthalen-2-ylmethoxy)-4-[4-(2-[1,2,4]-triazol-1-yl-ethoxy)-phenyl]-piperidine hydrochloride as a white powder, MS: 428 (M)⁺, from tert-butyl (3RS,4RS)-3-naphthalen-2-ylmethoxy-4-[4-(2-[1,2,4]triazol-1-yl-ethoxy)-phenyl]-piperidine-1-carboxylate;

9)—(3RS,4RS)-4-[4-(2-benzyloxy-ethoxy)-phenyl]-3-(naphthalen-2-ylmethoxy)-piperidine, MS: 468 (M+H)⁺, from tert-butyl (3RS,4RS)-4-[4-(2-benzyloxy-ethoxy)-phenyl]-3-(naphthalen-2-ylmethoxy)-piperidine-1-carboxylate.

The BOC derivatives used as the starting materials were prepared as follows:

(a) A solution of 150 mg (0.314 mmol) of tert-butyl (3RS,4RS)-4-[4-(2-hydroxy-ethoxy)-phenyl]-3-(naphthalen-2-ylmethoxy)-piperidine-1-carboxylate, 3.7 mg (0.031 mmol, 0.1 eq.) of 4-dimethylaminopyridine, 65.9 mg (0.375 mmol, 1.2 eq.) of 4-methoxy-benzoyl chloride and 51.7 .mu.1 of triethylamine in 10 ml of methylene chloride was stirred at room temperature for 15 hours. This reaction solution was poured into 50 ml of an ice/water mixture and extracted three times with ethyl acetate. The organic phases were washed once with water and once with saturated sodium chloride solution, dried over magnesium sulfate, evaporated under reduced pressure and dried in a high vacuum. The crude product (208 mg) was separated on silica gel using a 95:5 mixture of hexane and acetone as the eluent. There were obtained 104 mg (51% of theory) of tert-butyl (3RS,4RS)-4-[4-[2-(4-methoxy-benzoyloxy)-ethoxy]-phenyl]-3-(naphthalen-2-ylmethoxy)-piperidine-1-carboxylate as a colorless oil; MS: 612 (M+H)⁺.

(b) In an analogous manner to that described in Example 22(k), by acylating (3RS,4RS)-4-[4-(2-hydroxy-ethoxy)-phenyl]-3-(naphthalen-2-ylmethoxy)-piperidine-1-carboxylate with 2-chlorobenzoyl chloride there was obtained tert-butyl (3RS,4RS)-4-[4-[2-(2-chloro-benzoyloxy)-ethoxy]-phenyl]-3-(naphthalen-2-ylmethoxy)-piperidine-1-carboxylate as a yellow oil; MS: 616 (M+H)⁺.

c) In an analogous manner to that described in Example 22(k), by acylating tert-butyl (3RS,4RS)-4-[4-(2-hydroxy-ethoxy)-phenyl]-3-(naphthalen-2-ylmethoxy)-piperidine-1-carboxylate with benzoyl chloride there was obtained tert-butyl (3RS,4RS)-4-[4-(2-benzoyloxy-ethoxy)-phenyl]-3-(naphthalen-2-ylmethoxy)-piperidine-1-carboxylate as a colorless, amorphous solid; R_{f}: 0.61 (SiO₂, hexane:acetone=95:5).

(d) In an analogous manner to that described in Example 22(k), by acylating tert-butyl (3RS,4RS)-4-[4-(2-hydroxy-ethoxy)-phenyl]-3-(naphthalen-2-ylmethoxy)-piperidine-1-carboxylate with 2-chlorocarbonyl-benzyl benzoate there was obtained tert-butyl (3RS,4RS)-4-[4-[2-(2-benzoyloxymethyl-benzoyloxy)-ethoxy]-phenyl)-3-(naphthalen-2-ylmethoxy)-piperidine-1-carboxylate as a colorless solid; MS: 716 (M+H)⁺.

(e) In an analogous manner to that described in Example 33(a), from tert-butyl (3RS,4RS)-4-[4-(2-hydroxy-ethoxy)-phenyl]-3-(naphthalen-2-ylmethoxy)-piperidine-1-carboxylate and diphenyl sulfide there was obtained tert-butyl (3RS,4RS)-3-(naphthalen-2-ylmethoxy)-4-[4-(2-phenylsulfanyl-ethoxy)-phenyl]-piperidine-1-carboxylate as a colorless solid; MS: 570 (M+H)⁺.

(f) In an analogous manner to that described in Example 33(c), by oxidizing tert-butyl (3RS,4RS)-3-(naphthalen-2-ylmethoxy)-4-[4-(2-phenylsulfanyl-ethoxy)-phenyl]-piperidine-1-carboxylate there was obtained tert-butyl (3RS,4RS)-3-(naphthalen-2-ylmethoxy)-4-[4-(2-phenylsulfonyl-ethoxy)-phenyl]-piperidine-1-carboxylate as a colorless foam; MS: 619 (M+NH₄)⁺.

(g) A solution of 478 mg (1.00 mmol) of tert-butyl (3RS,4RS)-4-[4-(2-hydroxy-ethoxy)-phenyl)-3-(naphthalen-2-ylmethoxy)-piperidine-1-carboxylate and 93 ml of mesyl chloride in 5 ml of pyridine was stirred at room temperature for 2 hours. Subsequently, the reaction solution was poured into 50 ml of an ice/water mixture and extracted three times with methylene chloride. The organic phases were washed twice with water, dried over magnesium sulfate, evaporated under reduced pressure and dried in a high vacuum. This yielded 569 mg of crude tert-butyl (3RS,4RS)-4-[4-(2-methylsulfonyloxy-ethoxy)-phenyl]-3-(naphthalen-2-ylmethoxy)-piperidine-1-carboxylate, which was used in the following step without further purification and characterization.

(h) A mixture of 569 mg of crude tert-butyl (3RS,4RS)-4-[4-(2-methylsulfonyloxy-ethoxy)-phenyl]-3-(naphthalen-2-ylmethoxy)-piperidine-1-carboxylate and 650 mg of sodium azide in 20 ml of dimethyl sulfoxide was stirred at 80° C. for 3.5 hours. Subsequently, this reaction solution was poured into 50 ml of an ice/water mixture and extracted three times with ethyl acetate. The organic phases were washed with water, dried over magnesium sulfate, evaporated under reduced pressure and dried in a high vacuum. The yellow oil (670 mg) was separated on silica gel using a 9:1 mixture of hexane and ethyl acetate as the eluent. There were obtained 271 mg (54% of theory over both steps) of tert-butyl (3RS,4RS)-4-[4-(2-azido-ethoxy)-phenyl]-3-(naphthalen-2-ylmethoxy)-piperidine-1-carboxylate as a colorless solid; MS: 503 (M+H)⁺.

(i) A mixture of 115.9 mg (0.231 mmol) of tert-butyl (3RS,4RS)-4-[4-(2-azido-ethoxy)-phenyl]-3-(naphthalen-2-ylmethoxy)-piperidine-1-carboxylate, 87.4 mg (0.330 mmol, 1.43 eq.) of triphenylphosphine and 6.1 ,l (6.1 mg, 0.339 mmol, 1.47 eq.) of deionized water was stirred at room temperature for 4 hours. Subsequently, 3 ml of acetic acid were added and the mixture was stirred at room temperature for 17 hours. This reaction mixture was poured into an ice/water mixture, made basic with saturated sodium hydrogen carbonate solution and extracted three times with ethyl acetate. The organic phases were washed once with water and once with saturated sodium chloride solution, dried over magnesium sulfate, evaporated under reduced pressure and dried in a high vacuum. This yielded 192.3 mg of crude tert-butyl (3RS,4RS)-4-[4-(2-amino-ethoxy)-phenyl]-3-(naphthalen-2-ylmethoxy)-piperidine-1-carboxylate, which was used in the following reaction without further purification; R_{f}: 0.10 (SiO₂, methylene chloride:acetone=95:5+0.1% ammonia)

j) A solution of 192.4 mg of crude tert-butyl (3RS,4RS)-4-[4-(2-amino-ethoxy)-phenyl]-3-(naphthalen-2-ylmethoxy)-piperidine-1-carboxylate in 3 ml of methylene chloride was treated at 0° C. with 51.7 ml of triethylamine and 64.9 mg (0.462 mmol, 2.0 eq.) of benzoyl chloride and the mixture was stirred at 0° C. for 0.75 hr and at room temperature for 3 hours. The reaction mixture was poured into an ice/water mixture, made basic with saturated sodium hydrogen carbonate solution and extracted three times with ethyl acetate. The organic phases were washed once with water and once with saturated sodium chloride solution, dried over magnesium sulfate, evaporated under reduced pressure and dried in a high vacuum. The colorless oil was separated on silica gel using a 95:5 mixture of methylene chloride and acetone as the eluent. There were obtained 44.9 mg (33% of theory) of tert-butyl (3RS,4RS)-4-[4-(2-benzoylamino-ethoxy)-phenyl]-3-(naphthalen-2-ylmethoxy)-piperidine-1-carboxylate in the form of a light yellow oil; MS: 581 (M+H)⁺.

(k) A solution of 110 mg (0.2 mmol) of crude tert-butyl (3RS,4RS)-4-[4-(2-methylsulfonyloxy-ethoxy)-phenyl]-3-(naphthalen-2-ylmethoxy)-piperidine-1-carboxylate in 5 ml of dimethylformamide was treated with 70 mg (1.0 mmol) of 1,2,4-triazole sodium salt and the reaction mixture was heated to 100° C. for 6 hours. Subsequently, the mixture was cooled to room temperature and the dimethylformamide was distilled off in an oil pump vacuum. The residue was taken up in 10 ml of methylene chloride, washed with 2 ml of water, the organic phase was dried over sodium sulfate and evaporated under reduced pressure. The residue was chromatographed on silica gel using a 95:5:0.1 mixture of methylene chloride, methanol and ammonia as the eluent. There were obtained 90 mg (85% of theory) of tert-butyl (3RS,4RS)-3-naphthalen-2-ylmethoxy-4-[4-(2-[1,2,4]triazol-1-yl-ethoxy)-phenyl]-piperidine-1-carboxylate as a colorless oil; MS: 529 (M+H)⁺.

(l) In analogy to the procedure described in Example 3(c), by alkylating tert-butyl (3RS,4RS)-4-[4-(2-hydroxy-ethoxy)-phenyl]-3-(naphthalen-2-ylmethoxy)-piperidine-1-carboxylate with benzyl bromide there was obtained tert-butyl (3RS,4RS)-4-[4-(2-benzyloxy-ethoxy)-phenyl]-3-(naphthalen-2-ylmethoxy)-piperidine-1-carboxylate; MS: 568 (M+H)⁺.

### Example 55

The following compounds were obtained in an analogous manner to that described in Example 22(l):

1)—(3RS,4RS)-2-{4-[3-(4-Hydroxy-naphthalen-2-ylmethoxy)-piperidin-4-yl]-phenoxy}-ethyl 2-chloromethyl-benzoate as a colorless oil from tert-butyl (3RS,4RS)-4-[4-[2-(2-chloromethyl-benzoyloxy)-ethoxy]-phenyl]-3-[4-(2-trimethylsilanyl-ethoxymethoxy)-naphthalen-2-ylmethoxy]-piperidine-1-carboxylate by simultaneous cleavage of the BOC and acetal groups;

2)—(3RS,4RS)-2-[4-[3-[4-(2-methoxy-benzyloxy)-naphthalen-2-ylmethoxy]-piperidin-4-yl]-phenoxy]-ethyl benzoate hydrochloride as a colorless solid, MS: 618 (M+H)⁺, from tert-butyl (3RS,4RS)-4-[4-(2-benzoyloxy-ethoxy)-phenyl]-3-[4-(2-methoxy-benzyloxy)-naphthalen-2-ylmethoxy]-piperidine-1-carboxylate;

3)—(3RS,4RS)-4-[4-(2-benzyloxy-ethoxy)-phenyl]-3-(4-benzyloxy-naphthalen-2-ylmethoxy)-piperidine, MS: 574 (M+H)⁺, from tert-butyl (3RS,4RS)-4-[4-(2-benzyloxy-ethoxy)-phenyl]-3-(4-benzyloxy-naphthalen-2-yl methoxy)-piperidine-1-carboxylate.

The BOC derivatives used as the starting materials were prepared as follows:

(a) In an analogous manner to that described in Example 44(e), by alkylating a mixture of tert-butyl (3RS,4RS)-3-hydroxy-4-[4-[2-[(RS)- and -[(SR)-tetrahydro-pyran-2-yloxy]-ethoxy]-phenyl]-piperidine-1-carboxylate with 3-chloromethyl-1-(2-trimethylsilanyl-ethoxymethoxy)-naphthalene there was obtained a mixture of tert-butyl (3RS,4RS)-4-[4-[2-[(RS)- and -[(SR)-tetrahydro-pyran-2-yl]-ethoxy]-phenyl]-3-[4-(2-trimethylsilanyl-ethoxymethoxy)-naphthalen-2-ylmethoxy]-piperidine-1-carboxylate as a colorless oil; MS: 725 (M+NH₄)⁺.

(b) In an analogous manner to that described in Example 53(c), from a mixture of tert-butyl (3RS,4RS)-4-[4-[2-[(RS)- and -[(SR)-tetrahydro-pyran-2-yl]-ethoxy]-phenyl]-3-[4-(2-trimethylsilanyl-ethoxymethoxy)-naphthalen-2-ylmethoxy]-piperidine-1-carboxylate there was obtained tert-butyl (3RS,4RS)-4-[4-(2-hydroxy-ethoxy)-phenyl]-3-[4-(2-trimethylsilanyl-ethoxymethoxy)-naphthalen-2-ylmethoxy]-piperidine-1-carboxylate as a colorless solid; MS: 624 (M+H)⁺.

(c) In an analogous manner to that described in Example 22(k), by acylating tert-butyl (3RS,4RS)-4-[4-(2-hydroxy-ethoxy)-phenyl]-3-[4-(2-trimethylsilanyl-ethoxymethoxy)-naphthalen-2-ylmethoxy]-piperidine-1-carboxylate with 2-chloromethyl-benzoyl chloride there was obtained tert-butyl (3RS,4RS)-4-[4-[2-(2-chloromethyl-benzoyloxy)-ethoxy]-phenyl]-3-[4-(2-trim ethylsilanyl-ethoxymethoxy)-naphthalen-2-ylmethoxy]-piperidine-1-carboxylate as a colorless solid; MS: 776 (M+H)⁺.

(d) 200 mg (0.28 mmol) of a mixture of tert-butyl (3RS,4RS)-4-[4-[2-[(RS)- and -[(SR)-tetrahydro-pyran-2-yl]-ethoxy]-phenyl]-3-[4-(2-trimethylsilanyl-ethoxymethoxy)-naphthalen-2-ylmethoxy]-piperidine-1-carboxylate were dissolved in 7.6 ml 0.1 M hydrogen chloride in methanol and the mixture was stirred at room temperature for 2 hours. Subsequently, a further 0.36 ml of 2 M hydrogen chloride in methanol was added and the mixture was stirred at room temperature for a further 2 hours. This reaction solution was poured into semi-saturated sodium hydrogen carbonate solution and extracted three times with ethyl acetate. The organic phases were washed once with water and once with saturated sodium chloride solution, dried over magnesium sulfate, evaporated under reduced pressure and dried in a high vacuum. The yellowish oil (204 mg) was separated on silica gel using a 1:1 mixture of hexane and ethyl acetate (extracted against concentrated ammonia). This yielded 111 mg (80% of theory) of tert-butyl (3RS,4RS)-4-[4-(2-hydroxy-ethoxy)-phenyl]-3-(4-hydroxy-naphthalen-2-ylmethoxy)-piperidine-1-carboxylate in the form of a colorless oil; R: 0.26 (SiO₂, hexane:ethyl acetate=: 1).

(e) In an analogous manner to that described in Example 44(e), by alkylating tert-butyl (3RS,4RS)-4-[4-(2-hydroxy-ethoxy)-phenyl]-3-(4-hydroxy-naphthalen-2-ylmethoxy)-piperidine-1-carboxylate with 2-methoxybenzyl chloride there was obtained tert-butyl (3RS,4RS)-4-[4-(2-hydroxy-ethoxy)-phenyl]-3-[4-(2-methoxy-benzyloxy)-naphthalen-2-ylmethoxy]-piperidine-1-carboxylate as a colorless oil; MS: 652 (M+K)⁺.

(f) In an analogous manner to that described in Example 22(k), by acylating tert-butyl (3RS,4RS)-4-[4-(2-hydroxy-ethoxy)-phenyl]-3-[4-(2-methoxy-benzyloxy)-naphthalen-2-ylmethoxy]-piperidine-1-carboxylate with benzoyl chloride there was obtained tert-butyl (3RS,4RS)-4-[4-(2-benzoyloxy-ethoxy)-phenyl]-3-[4-(2-methoxy-benzyloxy)-naphthalen-2-ylmethoxy]-piperidine-1-carboxylate as a colorless oil; MS: 736 (M+H)⁺.

(g) In an analogous manner to that described in Example 1(g), by two-fold alkylation of tert-butyl (3RS,4RS)-4-[4-(2-hydroxy-ethoxy)-phenyl]-3-(4-hydroxy-naphthalen-2-ylmethoxy)-piperidine-1-carboxylate with benzyl bromide there was obtained tert-butyl (3RS,4RS)-4-[4-(2-benzyloxy-ethoxy)-phenyl]-3-(4-benzyloxy-naphthalen-2-yl methoxy)-piperidine-1-carboxylate.

### Example 56

The following compounds were obtained in an analogous manner to that described in Example 22(l) by simultaneous cleavage of the BOC and SEM groups using acid:

1)—(3RS,4RS)-2-[4-(3-Naphthalen-2-ylmethoxy-piperidin-4-yl)-phenoxy]-ethyl 4-hydroxy-benzoate hydrochloride as a colorless solid, MS: 498 (M+H)⁺, from tert-butyl (3RS,4RS)-3-(naphthalen-2-ylmethoxy)-4-[4-[2-[4-(2-trimethylsilanyl-ethoxy methoxy)-benzoyloxy]-ethoxy]-phenyl]-piperidine-1-carboxylate;

2)—(3RS,4RS)-3-[4-[4-(2-benzyloxy-ethoxy)-phenyl]-piperidin-3-yloxymethyl}-naphthalen-1-ol, MS: 484 (M+H)⁺, from tert-butyl (3RS,4RS)-4-[4-(2-benzyloxy-ethoxy)-phenyl]-3-(4-hydroxy-naphthalen-2-ylmethoxy)-piperidine-1-carboxylate.

The BOC derivatives used as the starting materials were prepared as follows:

(a) In an analogous manner to that described under Example 24(l), from tert-butyl (3RS,4RS)-4-[4-(2-hydroxy-ethoxy)-phenyl]-3-(naphthalen-2-ylmethoxy)-piperidine-1-carboxylate and 4-(2-trimethylsilanyl-ethoxymethoxy)-benzoic acid using N-ethyl-N′-(3-dimethylaminopropyl)-carbodiimide hydrochloride (EDC) as the condensation agent there was obtained tert-butyl (3RS,4RS)-3-(naphthalen-2-ylmethoxy)-4-[4-[2-[4-(2-trimethylsilanyl-ethoxy methoxy)-benzoyloxy]-ethoxy]-phenyl]-piperidine-1-carboxylate as a colorless, amorphous solid; MS: 728 (M+H)⁺.

(b) In an analogous manner to that described in Example 1 (g), by alkylating tert-butyl (3RS,4RS)-4-[4-(2-hydroxy-ethoxy)-phenyl)-3-[4-(2-trimethylsilanyl-ethoxymethoxy)-naphthalen-2-ylmethoxy)-piperidine-1-carboxylate with benzyl bromide there was obtained tert-butyl (3RS,4RS)-4-[4-(2-benzyloxy-ethoxy)-phenyl]-3-(4-hydroxy-naphthalen-2-ylmethoxy)-piperidine-1-carboxylate.

### Example 57

(a) In an analogous manner to that described in Example 44(e), by alkylating tert-butyl (3RS,4RS)-3-hydroxy-4-(4-hydroxy-phenyl)-piperidine-1-carboxylate with 2-(3-bromo-propoxy)-tetrahydro-pyran there was obtained a mixture of tert-butyl (3RS,4RS)-3-hydroxy-4-[4-[3-[(RS)- and -[(SR)-tetrahydro-pyran-2-yloxy]-propoxy]-phenyl]-piperidine-1-carboxylate as a colorless solid; R_{f}: 0.23 (SiO₂, hexane:ethyl acetate=4:1).

(b) Alkylation of a mixture of tert-butyl (3RS,4RS)-3-hydroxy-4-[4-[3-[(RS)- and -[(SR)-tetrahydro-pyran-2-yloxy)-propoxy]-phenyl]-piperidine-1-carboxylate with 2-bromomethylnaphthalene analogously to the procedure described in Example 22(i) gave a mixture of tert-butyl (3RS,4RS)-3-(naphthalen-2-ylmethoxy)-4-[4-[3-[(RS)- and -[(SR)-tetrahydro-pyran-2-yloxy]-propoxy]-phenyl]-piperidine-1-carboxylate as a yellow oil; R_{f}: 0.35 (SiO₂, hexane:acetone=4:1).

(c) A solution of 5.22 g of the crude mixture of tert-butyl (3RS,4RS)-3-(naphthalen-2-ylmethoxy)-4-[4-[3-[(RS)- and -[(SR)-tetrahydro-pyran-2-yloxy]-propoxy]-phenyl]-piperidine-1-carboxylate in 30 ml of tetrahydrofuran and 30 ml of 2 N hydrochloric acid was stirred at room temperature for 14 hours and at 40° C. for 2 hours. The tetrahydrofuran was subsequently distilled off under reduced pressure and the residual aqueous phase was extracted three times with methylene chloride. The organic phase was dried over magnesium sulfate and thereafter the solvent was distilled off under reduced pressure. The resulting yellow oil (2.9 g) was chromatographed on silica gel using a 9:1 mixture of methylene chloride and ethyl acetate as the eluent. There were obtained 1.08 g (34% of theory over both steps) of tert-butyl (3RS,4RS)-4-[4-(3-hydroxy-propoxy)-phenyl]-3-(naphthalen-2-ylmethoxy)-piperidine-1-carboxylate in the form of a white amorphous solid; MS: 491 (M)⁺.

(d) Reaction of tert-butyl (3RS,4RS)-4-[4-(3-hydroxy-propoxy)-phenyl]-3-(naphthalen-2-ylmethoxy)-piperidine-1-carboxylate with mesyl chloride analogously to the procedure described in Example 54(h) gave tert-butyl (3RS,4RS)-4-[4-(3-methylsulfonyloxy-propoxy)-phenyl]-3-(naphthalen-2-ylmethoxy)-piperidine-1-carboxylate as a yellow oil; R_{f}: 0.50 (SiO₂, hexane:ethyl acetate=1:1).

(e) A solution of 850 mg of crude tert-butyl (3RS,4RS)-4-[4-(3-methylsulfonyloxy-propoxy)-phenyl]-3-(naphthalen-2-ylmethoxy)-piperidine-1-carboxylate in 6.1 ml (48.98 mmol, 32.4 eq.) of a 33% methylamine solution in ethanol was stirred at room temperature for 14 hours. Thereupon, the solution was evaporated under reduced pressure. The yellow solid (932 mg) was chromatographed on silica gel using a 90:10:0.1 mixture of methylene chloride, methanol and ammonia as the eluent. This yielded 610 mg (80% of theory over both steps) of tert-butyl (3RS,4RS)-4-[4-(3-methylamino-propoxy)-phenyl]-3-(naphthalen-2-ylmethoxy)-piperidine-1-carboxylate as a colorless solid; MS: 505 (M+H)⁺.

(f) Acylation of tert-butyl (3RS,4RS)-4-[4-(3-methylamino-propoxy)-phenyl]-3-(naphthalen-2-ylmethoxy)-piperidine-1-carboxylate with benzoyl chloride analogously to the procedure described in Example 22(k) gave tert-butyl (3RS,4RS)-4-[4-[3-(benzoyl-methyl-amino)-propoxy]-phenyl]-3-(naphthalen-2-ylmethoxy)-piperidine-1-carboxylate as a colorless oil; MS: 609 (M+H)⁺.

(g) In an analogous manner to the procedure described in Example 22(l), from tert-butyl (3RS,4RS)-4-[4-[3-(benzoyl-methyl-amino)-propoxy]-phenyl]-3-(naphthalen-2-ylmethoxy)-piperidine-1-carboxylate by cleavage of the BOC group there was obtained (3RS,4RS)-N-methyl-N-[3-[4-[3-(naphthalen-2-yloxy)-piperidine-4-yl]-phenoxy]-propyl]-benzamide as a colorless solid; MS: 509 (M+H)⁺.

### Example 58

The following compounds were obtained in analogy to the procedure described in Example 8(g) by cleavage of the BOC group using hydrochloric acid in methanol:

1)—(3RS,4RS)-2-[4-(3-Naphthalen-2-ylmethoxy-piperidin-4-yl)-phenoxy]-ethyl pyridine-3-carboxylate hydrochloride as a colorless solid, MS: 483 (M+H)⁺, from 2-[4-[1-tert-butoxycarbonyl-3-(naphthalen-2-ylmethoxy)-piperidin-4-yl]-phenoxy}-ethyl (3RS,4RS)-nicotinate;

2)—(3RS,4RS)-2-[4-(3-naphthalen-2-ylmethoxy-piperidin-4-yl)-phenoxy]-ethyl 1,3-benzodioxole-5-carboxylate hydrochloride as a colorless solid, MS: 526 (M+H)⁺, from tert-butyl (3RS,4RS)-4-{4-[2-(benzo[1,3]dioxol-5-carbonyloxy)-ethoxy]-phenyl}-3-(naphthalen-2-ylmethoxy)-piperidine-1-carboxylate;

3)—(3RS,4RS)-2-[4-(3-naphthalen-2-ylmethoxy-piperidin-4-yl)-phenoxy]-ethyl thiophene-3-carboxylate hydrochloride as a colorless solid, MS: 488 (M+H)⁺, from tert-butyl (3RS,4RS)-3-(naphthalen-2-ylmethoxy)-4-{4-[2-(thiophene-3-carbonyloxy)-ethoxy]-phenyl}-piperidine-1-carboxylate;

4)—(3RS,4RS)-2-[4-(3-naphthalen-2-ylmethoxy-piperidin-4-yl)-phenoxy]-ethyl thiophene-2-carboxylate hydrochloride as a colorless solid, MS: 488 (M+H)⁺, from tert-butyl (3RS,4RS)-3-(naphthalen-2-ylmethoxy)-4-[4-[2-(thiophene-2-carbonyloxy)-ethoxy]-phenyl]-piperidine-1-carboxylate;

5)—2-[(3RS,4RS)-4-[3-(naphthalen-2-ylmethoxy)-piperidin-4-yl]-phenoxy]-ethyl furan-3-carboxylate hydrochloride as a colorless solid, MS: 472 (M+H)⁺, from tert-butyl 4-[4-[2-(furan-3-carbonyloxy)-ethoxy]-phenyl]-3-(naphthalen-2-ylmethoxy)-piperidin-1-carboxylate;

6)—2-[(3RS,4RS)-4-[3-(naphthalen-2-ylmethoxy)-piperidin-4-yl]-phenoxy]-ethyl furan-2-carboxylate hydrochloride as a colorless solid, MS: 472 (M+H)⁺, from tert-butyl 4-[4-[2-(furan-2-carbonyloxy)-ethoxy]-phenyl}-3-(naphthalen-2-ylmethoxy)-piperidine-1-carboxylate;

7)—a mixture of (3RS,4RS)-2-[4-(2-naphthalen-2-ylmethoxy-piperidin-4-yl)-phenoxy]-ethyl (RS)- and (SR)-methoxy-phenyl-acetate hydrobromide as a brownish solid, MS: 526 (M+H)⁺, from a mixture of tert-butyl (3RS,4RS)-4-[4-[2-[(RS)- and (SR)-methoxyphenyl-acetoxy]ethoxy]phenyl]-3-(naphthalen-2-ylmethoxy)-piperidine-1-carboxylate;

8)—(3RS,4RS)-2-[4-(3-naphthalen-2-yl-methoxy-piperidin-4-yl)-phenoxy]-ethyl 2-methylsulfanyl-benzoate hydrochloride as a beige colored solid, MS: 528 (M+H)⁺, from tert-butyl (3RS,4RS)-4-[4-[2-(2-methylsulfanyl-benzoyloxy)-ethoxy]-phenyl]-3-(naphthalen-2-ylmethoxy)-piperidine-1-carboxylate;

9)—(3RS,4RS)-2-[4-(2-naphthalen-2-ylmethoxy-piperidin-4-yl)-phenoxy]-ethyl (RS)- and (SR)-2-methylsulfinyl-benzoate hydrochloride as a colorless solid, MS: 544 (M+H)⁺, from tert-butyl (3RS,4RS))-4-{4-[2-[(RS)- and (SR)-2-methylsulfinyl-benzoyloxy]-ethoxy]-phenyl}-3-(naphthalen-2-ylmethoxy)-piperidine-1-carboxylate.

The BOC derivatives used as the starting materials were prepared as follows in an analogous manner to that described under Example 24 (l) using N-ethyl-N′-(3-dimethylaminopropyl)-carbodiimide hydrochloride (EDC) as the condensation agent:

(a) 2-[4-[1-tert-Butoxycarbonyl-3-(naphthalen-2-ylmethoxy)-piperidin-4-yl]-phenoxy]-ethyl (3RS,4RS)-nicotinate as a colorless solid, MS: 481 (M-C₄H₉COO)⁺, from tert-butyl (3RS,4RS)-4-[4-(2-hydroxy-ethoxy)-phenyl]-3-(naphthalen-2-ylmethoxy)-piperidine-1-carboxylate and pyridine-3-carboxylic acid.

(b) tert-Butyl (3RS,4RS)-4-{4-[2-(benzo[1,3]dioxol-5-carbonyloxy)-ethoxy]-phenyl}-3-(naphthalen-2-ylmethoxy)-piperidine-1-carboxylate as a colorless solid, MS: 626 (M+H)⁺, from tert-butyl (3RS,4RS)-4-[4-(2-hydroxy-ethoxy)-phenyl]-3-(naphthalen-2-ylmethoxy)-piperidine-1-carboxylate and piperonylic acid.

(c) tert-Butyl (3RS,4RS)-3-(naphthalen-2-ylmethoxy)-4-{4-[2-(thiophen-3-carbonyloxy)-ethoxy]-phenyl]-piperidine-1-carboxylate as a colorless solid, MS: 587 (M)⁺, from tert-butyl (3RS,4RS)-4-[4-(2-hydroxy-ethoxy)-phenyl]-3-(naphthalen-2-ylmethoxy)-piperidine-1-carboxylate and thiophene-3-carboxylic acid.

(d) tert-Butyl (3RS,4RS)-3-(naphthalen-2-ylmethoxy)-4-[4-[2-(thiophen-2-carbonyloxy)-ethoxy]-phenyl]-piperidine-1-carboxylate as a colorless solid, MS: 588 (M+H)⁺, from tert-butyl (3RS,4RS)-4-[4-(2-hydroxy-ethoxy)-phenyl]-3-(naphthalen-2-ylmethoxy)-piperidine-1-carboxylate and thiophene-2-carboxylic acid.

(e) tert-Butyl 4-[4-[2-(furan-3-carbonyloxy)-ethoxy]-phenyl]-3-(naphthalen-2-ylmethoxy)-piperidine-1-carboxylate as a colorless solid, MS: 572 (M+H)⁺, from tert-butyl (3RS,4RS)-4-[4-(2-hydroxy-ethoxy)-phenyl]-3-(naphthalen-2-ylmethoxy)-piperidine-1-carboxylate and furan-3-carboxylic acid.

(f) tert-Butyl 4-[4-[2-(furan-2-carbonyloxy)-ethoxy]-phenyl}-3-(naphthalen-2-ylmethoxy)-piperidine-1-carboxylate as a colorless solid, MS: 572 (M+H)⁺, from tert-butyl (3RS,4RS)-4-[4-(2-hydroxy-ethoxy)-phenyl]-3-(naphthalen-2-ylmethoxy)-piperidine-1-carboxylate and furan-2-carboxylic acid.

(g) tert-Butyl (3RS,4RS)-4-[4-[2-(methoxy-phenyl-acetoxy)-ethoxy]-phenyl]-3-(naphthalen-2-ylmethoxy)-piperidine-1-carboxylate as a colorless oil, MS: 648 (M+Na)⁺, from tert-butyl (3RS,4RS)-4-[4-(2-hydroxy-ethoxy)-phenyl]-3-(naphthalen-2-ylmethoxy)-piperidine-1-carboxylate and (RS)-.alpha.-methoxy-phenylacetic acid.

(h) tert-Butyl (3RS,4RS)-4-[4-[2-(2-methylsulfanyl-benzoyloxy)-ethoxy]-phenyl]-3-(naphthalen-2-ylmethoxy)-piperidine-1-carboxylate as a colorless solid, MS: 628 (M+H)⁺, from tert-butyl (3RS,4RS)-4-[4-(2-hydroxy-ethoxy)-phenyl]-3-(naphthalen-2-ylmethoxy)-piperidine-1-carboxylate and 2-methylsulfanylbenzoic acid.

(i) A solution of 170 mg (0.8 mmol) of sodium metaperiodate in 2 ml of water was added to a solution of 250 mg (0.4 mmol) of tert-butyl (3RS,4RS)-4-[4-[2-(2-methylsulfanyl-benzoyloxy)-ethoxy]-phenyl]-3-(naphthalen-2-ylmethoxy)-piperidine-1-carboxylate in 25 ml of methanol. The resulting reaction mixture was stirred at 50° C. for 8 hours. Thereafter, the solvent was distilled off under reduced pressure, the residue was partitioned between ethyl acetate and water, the organic phase was dried over sodium sulfate and evaporated under reduced pressure. There were obtained 230 mg (90% of theory) of crude tert-butyl (3RS,4RS))-4-{4-[2-[(RS)- and (SR)-2-methylsulfinyl-benzoyloxy]-ethoxy]-phenyl}-3-(naphthalen-2-ylmethoxy)-piperidine-1-carboxylate as a colorless solid; MS: 644 (M+H)⁺.

### Example 59

The following compounds were obtained in an analogous manner to that described in Example 22(l) by cleavage of the BOC group:

1)—(3RS,4RS)-4-[4-(3-Morpholin-4-yl-propoxy)-phenyl]-3-(naphthalen-2-ylmethoxy)-piperidine as a colorless solid, MS: 461 (M+H)⁺, from tert-butyl (3RS,4RS)-4-[4-(3-morpholin-4-yl-propoxy)-phenyl]-3-(naphthalen-2-ylmethoxy)-piperidine-1-carboxylate;

2)—(3RS,4RS)-3-[4-(3-naphthalen-2-ylmethoxy-piperidin-4-yl)-phenoxy]-propyl 2,2-dimethyl-propionate as a colorless, amorphous solid, MS: 476 (M+H)⁺, from tert-butyl (3RS,4RS)-4-[4-[3-(2,2-dimethyl-propionyloxy)-propoxy]-phenyl]-3-(naphthalen-2-ylmethoxy)-piperidine-1-carboxylate;

3)—(3RS,4RS)-3-[4-(3-napthalen-2-ylmethoxy-piperidin-4-yl)-phenoxy]-propyl benzoate hydrochloride as a colorless solid, MS: 496 (M+H)⁺, from tert-butyl (3RS,4RS)-4-[4-(3-benzoyloxy-propoxy)-phenyl]-3-(naphthalen-2-ylmethoxy)-piperidine-1-carboxylate;

4)—(3RS,4RS)-4-(4-benzyloxy-phenyl)-3-(naphthalen-2-yloxy)-piperidine as a colorless, amorphous solid, MS: 424 (M+H)⁺, from tert-butyl (3RS,4RS)-4-(4-benzyloxy-phenyl)-3-(naphthalen-2-yloxy)-piperidine-1-carboxylate;

5)—(3RS,4RS)-3-(2-methoxy-benzyloxy)-4-(4-naphthalen-2-ylmethoxy-phenyl)-piperidine hydrochloride as a colorless solid, MS: 454 (M+H)⁺, from tert-butyl (3RS,4RS)-3-(2-methoxy-benzyloxy)-4-[4-(naphthalen-2-ylmethoxy)-phenyl]-piperidine-1-carboxylate

The BOC derivatives used as the starting materials were prepared as follows:

(a) A solution of 200 mg (0.35 mmol) of tert-butyl 3RS,4RS)-4-[4-(3-methylsulfonyloxy-propoxy)-phenyl]-3-(naphthalen-2-ylmethoxy)-piperidine-1-carboxylate in 1 ml of ethyl acetate was treated with 60 .mu.1 of morpholine and boiled under reflux for 3 hours. Subsequently, the reaction solution was diluted with 5 ml of ethyl acetate and extracted twice with 1 ml of saturated sodium hydrogen carbonate solution each time. The organic phases were dried over magnesium sulfate, evaporated under reduced pressure and dried in a high vacuum. The colorless oil (169 mg) was chromatographed on silica gel using ethyl acetate and the eluent. There were obtained 142 mg of tert-butyl (3RS,4RS)-4-[4-(3-morpholin-4-yl-propoxy)-phenyl]-3-(naphthalen-2-ylmethoxy)-piperidine-1-carboxylate in the form of white crystals; MS: 561 (M+H)⁺.

(b) In an analogous manner to that described in Example 22(k), by acylating tert-butyl (3RS,4RS)-4-[4-(3-hydroxy-propoxy)-phenyl]-3-(naphthalen-2-ylmethoxy)-piperidine-1-carboxylate with pivaloyl chloride there was obtained tert-butyl (3RS,4RS)-4-[4-[3-(2,2-dimethyl-propionyloxy)-propoxy]-phenyl]-3-(naphthalen-2-ylmethoxy)-piperidine-1-carboxylate as a colorless oil; MS: 576 (M+H)⁺.

(c) In an analogous manner to that described in Example 22(k), by acylating (3RS,4RS)-4-[4-(3-hydroxy-propoxy)-phenyl]-3-(naphthalen-2-ylmethoxy)-piperidine-1-carboxylate with benzoyl chloride there was obtained tert-butyl (3RS,4RS)-4-[4-(3-benzoyloxy-propoxy)-phenyl]-3-(naphthalen-2-ylmethoxy)-piperidine-1-carboxylate as a colorless oil; R_{f}: 0.84 (SiO₂, hexane:ethyl acetate=1:1). (d) A mixture of 1.0 g (3.41 mmol) of tert-butyl (3RS,4RS)-3-hydroxy-4-(4-hydroxy-phenyl)-piperidine-1-carboxylate, 471 mg (34.1 mmol, 10 eq.) of potassium carbonate and 607 ml (5.11 mmol, 1.5 eq.) of benzyl bromide in 30 ml of dimethylformamide was stirred at room temperature for 2 hours and at 80° C. for 15 hours. Subsequently, 471 mg (34.1 mmol, 10 eq.) of potassium carbonate and 607 ml (5.11 mmol, 1.5 eq.) of benzyl bromide were again added and the mixture was stirred at 80° C. for a further 6 hours. The reaction mixture, cooled to room temperature, was poured into 300 ml of an ice/water mixture and extracted three times with 250 ml of ethyl acetate. The organic phases were washed once with water and once with saturated sodium chloride solution, dried over magnesium sulfate, evaporated under reduced pressure and dried in a high vacuum. The yellow oil (1.82 g) was separated on silica gel using an eluent gradient of 4:1 to 3:2 of a mixture of hexane and ethyl acetate as the eluent. There were obtained 620 mg (72% of theory) of tert-butyl (3RS,4RS)-4-(4-benzyloxy-phenyl)-3-hydroxy-piperidine-1-carboxylate which, after recrystallization from a mixture of methylene chloride and hexane, gave 361 mg (28% of theory) as a white, crystalline product; MS: 383 (M)⁺.

(e) In an analogous manner to that described in Example 22(i), by alkylating tert-butyl (3RS,4RS)-4-(4-benzyloxy-phenyl)-3-hydroxy-piperidine-1-carboxylate with 2-bromomethylnaphthalene there was obtained tert-butyl (3RS,4RS)-4-(4-benzyloxy-phenyl)-3-(naphthalen-2-yloxy)-piperidine-1-carboxylate as a colorless, amorphous solid; MS: 523 (M)⁺.

(f) In analogy to the procedure described in Example 14, by alkylating tert-butyl (3RS,4RS)-3-hydroxy-4-(4-hydroxy-phenyl)-piperidine-1-carboxylate with 2-bromomethylnaphthalene in the presence of potassium carbonate there was obtained tert-butyl (3RS,4RS)-3-hydroxy-4-[4-(naphthalen-2-ylmethoxy)-phenyl]-piperidine-1-carboxylate as a colorless solid; MS: 434 (M+H)⁺.

(g) In analogy to the procedure described in Example 3(c), by alkylating tert-butyl (3RS,4RS)-3-hydroxy-4-[4-(naphthalen-2-ylmethoxy)-phenyl]-piperidine-1-carboxylate with 2-methoxybenzyl chloride there was obtained tert-butyl (3RS,4RS)-3-(2-methoxy-benzyloxy)-4-[4-(naphthalen-2-ylmethoxy)-phenyl]-piperidine-1-carboxylate as a colorless solid; MS: 554 (M+H)⁺.

### Example 60

The following compounds were obtained in an analogous manner to that described in Example 25(b) by cleavage of the 2,2,2-trichloroethyl carbamate by treatment with zinc in glacial acetic acid:

1)—3-{4-[3-(Naphthalen-2-ylmethoxy)-piperidin-4-yl]-phenoxy}-propyl (3RS,4RS)-carbamate as a colorless solid, MS: 435 (M+H)⁺, from 2,2,2-trichloro-ethyl (3RS,4RS)-4-[4-(3-carbamoyloxy-propoxy)-phenyl]-3-(naphthalen-2-ylmethoxy)-piperidine-1-carboxylate;

2)—3-{4-[3-(naphthalen-2-ylmethoxy)-piperidin-4-yl]-phenoxy}-propyl (3RS,4RS)-pyridin-2-yl-carbamate as a colorless oil, MS: 512 (M+H)⁺, from 2,2,2-trichloro-ethyl (3RS,4RS)-3-(naphthalen-2-ylmethoxy)-4-{4-[3-(pyridin-2-ylcarbamoyloxy)-propoxy]-phenyl}-piperidine-1-carboxylate;

3)—(3RS,4RS)-2-[3-[3-(naphthalen-2-ylmethoxy)-piperidin-4-yl]-phenoxy]-ethyl pyridin-2-yl-carbamate as a colorless oil, MS: 498 (M+H)⁺, from 2,2,2-trichloro-ethyl (3RS,4RS)-3-(naphthalen-2-ylmethoxy)-4-[3-(2-pyridin-2-ylcarbamoyloxy-ethoxy]-phenyl]-piperidine-1-carboxylate;

4)—(3RS,4RS)-2-[3-[3-(naphthalen-2-ylmethoxy)-piperidin-4-yl]-phenoxy]-ethyl carbamate as a colorless, amorphous solid, MS: 421 (M+H)⁺, from 2,2,2-trichloro-ethyl (3RS,4RS)-4-[3-(2-carbamoyloxy-ethoxy)-phenyl]-3-(naphthalen-2-ylmethoxy)-piperidine-1-carboxylate;

5)—(3RS,4RS)-2-[3-[3-(naphthalen-2-ylmethoxy)-piperidin-4-yl]-phenoxy]-ethanol as a colorless, amorphous solid, MS: 378 (M+H)⁺, from the mixture of 2,2,2-trichloro-ethyl (3RS,4RS)-3-(naphthalen-2-ylmethoxy)-4-[3-[2-[(RS)- and -[(SR)-tetrahydro-pyran-2-yloxy]-ethoxy]-phenyl]-piperidine-1-carboxylate with simultaneous cleavage of the THP group;

6)—(3RS,4RS)-2-[4-(3-naphthalen-2-ylmethoxy-piperidin-4-yl]-phenoxy]-ethyl benzoate as a colorless, amorphous solid, MS: 482 (M+H)⁺, from 2,2,2-trichloroethyl (3RS,4RS)-4-[3-(2-benzoyloxy-ethoxy)-phenyl]-3-(naphthalen-2-ylmethoxy)-piperidine-1-carboxylate;

7)—3-{3-[3-(naphthalen-2-ylmethoxy)-piperidin-4-yl]-phenoxy}-propyl (3RS,4RS)-pyridin-2-yl-carbamate as a colorless, amorphous solid, MS: 512 (M+H)⁺, from 2,2,2-trichloroethyl (3RS,4RS)-3-(naphthalen-2-ylmethoxy)-4-{3-[3-(pyridin-2-ylcarbamoyloxy)-propoxy]-phenyl}-piperidine-1-carboxylate;

8)—(3RS,4RS)-4-[3-[3-(naphthalen-2-yloxy)-piperidin-4-yl]-phenoxy]-butyl benzoate as a colorless, amorphous solid, MS: 510 (M+H)⁺, from 2,2,2-trichloro-ethyl (3RS,4RS)-4-[3-(4-benzoyloxy-butoxy)-phenyl]-3-(naphthalen-2-ylmethoxy)-piperidine-1-carboxylate.

The 2,2,2-trichloroethyl carbamates used as the starting materials were prepared as follows:

The following procedure was carried out in an analogous manner to that described in Example 22(a)-(c):

(a) 1-Benzyl-4-(3-methoxy-phenyl)-piperidin-4-ol was obtained as a colorless solid, R_{f}: 0.18 (SiO₂, methylene chloride:ethyl acetate=1:1), from 1-benzyl-4-piperidone and 3-iodoanisole. Subsequent elimination with p-toluenesulfonic acid yielded 1-benzyl-4-(3-methoxy-phenyl)-1,2,3,6-tetrahydro-pyridine as a colorless solid, MS: 279 (M)⁺. Hydroboration which followed gave 1-benzyl-4-(3-methoxy-phenyl)-piperidin-3-ol as a colorless powder, MS: 297 (M)⁺.

The following procedure was followed in an analogous manner to that described in Example 44 (d)-(i):

(b) From 1-benzyl-4-(3-methoxy-phenyl)-piperidin-3-ol by cleavage of the methyl ether (RS)- and (SR)-methoxy-phenyl-acetate boron tribromide in methylene chloride there was obtained (3RS,4RS)-1-benzyl-4-(3-hydroxy-phenyl)-piperidin-3-ol as a pale yellow solid; MS: 283 (M)⁺. Alkylation with rac.-2-(2-iodo-ethoxy)-tetrahydro-pyran in the presence of potassium carbonate gave a mixture of (3RS,4RS)-1-benzyl-4-[3-[2-[(RS)- and -[(SR)-tetrahydro-pyran-2-yloxy]-ethoxy]-phenyl]-piperidin-3-ol as a colorless oil; MS: 410 (M-H)⁺. By subsequent alkylation with 2-bromomethylnaphthalene in an analogous manner to that described in Example 22(i) there was obtained a mixture of (3RS,4RS)-1-benzyl-3-(naphthalen-2-ylmethoxy)-4-[3-[2-[(RS)- and -[(SR)-tetrahydro-pyran-2-yloxy]-ethoxy]-phenyl]-piperidine as a colorless oil; MS: 552 (M)⁺. Then, by cleavage of the benzyl group using 2,2,2-trichloroethyl chloroformate and potassium carbonate in an analogous manner to that described in Example 25(a) there was obtained a mixture of 2,2,2-trichloroethyl (3RS,4RS)-3-(naphthalen-2-ylmethoxy)-4-[3-[2-[(RS)- and -[(SR)-tetrahydro-pyran-2-yloxy]-ethoxy]-phenyl]-piperidine-1-carboxylate; R_{f}: 0.60 (SiO₂, methylene chloride:ethyl acetate=2:1). By subsequent cleavage of the THP group using p-toluenesulfonic acid there was obtained 2,2,2-trichloro-ethyl (3RS,4RS)-4-[3-(2-hydroxy-ethoxy)-phenyl]-3-(naphthalen-2-ylmethoxy)-piperidine-1-carboxylate. Finally, by reaction with pyridine-2-carbonyl azide in an analogous manner to that described in Example 24(m) there was obtained 2,2,2-trichloro-ethyl (3RS,4RS)-3-(naphthalen-2-ylmethoxy)-4-[3-(2-pyridin-2-ylcarbamoyloxy-ethoxy]-phenyl]-piperidine-1-carboxylate; R_{f}: 0.45 (SiO₂, methylene chloride:ethyl acetate=9:1).

(c) In an analogous manner to that described in Example 44(e), by alkylating (3RS,4RS)-1-benzyl-4-(4-hydroxy-phenyl)-piperidin-3-ol with rac.-2-(3-bromo-propoxy)-tetrahydro-pyran there was obtained (3RS,4RS)-1-benzyl-4-{4-[3-[(RS)- and (SR)-tetrahydro-pyran-2-yloxy]-propoxy]-phenyl}-piperidin-3-ol as a colorless solid; MS: 426 (M+H)⁺. Subsequent alkylation with 2-bromomethyl-naphthalene according to the procedure described in Example 12(b) gave (3RS,4RS)-1-benzyl-3-(naphthalen-2-ylmethoxy)-4-{4-[3-[(RS)- and (SR)-tetrahydro-pyran-2-yloxy]-propoxy]-phenyl}-piperidine as a colorless solid; MS: 566 (M+H)⁺. Further reaction with 2,2,2-trichloroethyl chloroformate analogously to Example 12(c) yielded 2,2,2-trichloro-ethyl (3RS,4RS)-3-(naphthalen-2-ylmethoxy)-4-{4-[3-[(RS)- and (SR)-tetrahydro-pyran-2-yloxy]-propoxy]-phenyl}-piperidine-1-carboxylate as a colorless solid; MS: 672 (M+Na)⁺. Cleavage of the THP group using p-toluenesulfonic acid analogously to Example 44(h) gave 2,2,2-trichloro-ethyl (3RS,4RS)-4-[4-(3-hydroxy-propoxy)-phenyl]-3-(naphthalen-2-ylmethoxy)-piperidine-1-carboxylate as a colorless solid; MS: 583 (M+NH₄)⁺. Finally, by reaction with sodium isocyanate in an analogous manner to that described in Example 24(m) there was obtained 2,2,2-trichloro-ethyl (3RS,4RS)-4-[4-(3-carbamoyloxy-propoxy)-phenyl]-3-(naphthalen-2-ylmethoxy)-piperidine-1-carboxylate as a colorless solid; MS: 609 (M+H)⁺.

(d) In an analogous manner to that described in Example 24(m), by reacting 2,2,2-trichloro-ethyl (3RS,4RS)-4-[4-(3-hydroxy-propoxy)-phenyl]-3-(naphthalen-2-ylmethoxy)-piperidine-1-carboxylate with pyridine-2-carbonyl azide there was obtained 2,2,2-trichloro-ethyl (3RS,4RS)-3-(naphthalen-2-ylmethoxy)-4-{4-[3-(pyridin-2-ylcarbamoyloxy)-propoxy]-phenyl}-piperidine-1-carboxylate as a colorless solid.

e) In an analogous manner to that described in Example 24(m), by reacting 2,2,2-trichloro-ethyl (3RS,4RS)-4-[3-(2-hydroxy-ethoxy)-phenyl]-3-(naphthalen-2-ylmethoxy)-piperidine-1-carboxylate with sodium isocyanate there was obtained 2,2,2-trichloro-ethyl (3RS,4RS)-4-[3-(2-carbamoyloxy-ethoxy)-phenyl]-3-(naphthalen-2-ylmethoxy)-piperidine-1-carboxylate as a colorless solid; R_{f}: 0.40 (SiO₂, methylene chloride:methanol=9:1).

(f) In an analogous manner to that described in Example 22(k), by acylating 2,2,2-trichloro-ethyl (3RS,4RS)-4-[3-(2-hydroxy-ethoxy)-phenyl]-3-(naphthalen-2-ylmethoxy)-piperidine-1-carboxylate with benzoyl chloride there was obtained 2,2,2-trichloroethyl (3RS,4RS)-4-[3-(2-benzoyloxy-ethoxy)-phenyl]-3-(naphthalen-2-ylmethoxy)-piperidine-1-carboxylate as a colorless oil.

(g) In an analogous manner to that described in Example 14, by alkylating (3RS,4RS)-1-benzyl-4-(3-hydroxy-phenyl)-piperidin-3-ol with rac.-2-(3-bromo-propoxy)-tetrahydro-pyran in the presence of potassium carbonate there was obtained a mixture of the diastereomeric racemates of (3RS,4RS)-1-benzyl-4-{3-[3-(tetrahydro-pyran-2-yloxy)-propoxy]-phenyl}-pip eridin-3-ol as a colorless oil; R_{f}: 0.38 (hexane:acetone=1:1). Subsequent alkylation with 2-bromomethylnaphthalene analogously to Example 12(b) gave a mixture of the diastereomeric racemates of 1-benzyl-3-(naphthalen-2-ylmethoxy)-4-{3-[3-(tetrahydro-pyran-2-yloxy)-propoxy]-phenyl}-piperidine as a colorless oil; MS: 566 (M+H)⁺. Subsequent reaction with 2,2,2-trichloroethyl chloroformate analogously to Example 12(c) yielded a mixture of the diastereomeric racemates of 2,2,2-trichloroethyl 3-(naphthalen-2-ylmethoxy)-4-{3-[3-(tetrahydro-pyran-2-yloxy)-propoxy]-phenyl}-piperidine-1-carboxylate which, without further purification and characterization, was reacted with p-toluenesulfonic acid analogously to Example 53(c) to give 2,2,2-trichloroethyl (3RS,4RS)-4-[3-(3-hydroxy-propoxy)-phenyl]-3-(naphthalen-2-ylmethoxy)-piperidine-1-carboxylate. Finally, by reaction with pyridine-2-carbonyl azide in an analogous manner to that described in Example 24(m) there was obtained 2,2,2-trichloroethyl (3RS,4RS)-3-(naphthalen-2-ylmethoxy)-4-{3-[3-(pyridin-2-ylcarbamoyloxy)-propoxy]-phenyl}-piperidine-1-carboxylate as a colorless oil; R_{f}: 0.55 (methylene chloride:ethyl acetate=1:1).

(h) In an analogous manner to that described in Example 14, by alkylating (3RS,4RS)-1-benzyl-4-(3-hydroxy-phenyl)-piperidin-3-ol with rac.-2-(4-bromo-butoxy)-tetrahydro-pyran [S. W. Baldwin et al., J. Org. Chem. 1985, 50, 4432-4439] and further reacting as described under (g) there was obtained 2,2,2-trichloro-ethyl (3RS,4RS)-4-[3-(4-hydroxy-butoxy)-phenyl]-3-(naphthalen-2-ylmethoxy)-piperidine-1-carboxylate as a colorless solid; R_{f}: 0.50 (methylene chloride:ethyl acetate=9:1). Subsequent acylation with benzoyl chloride analogously to Example 22(k) yielded 2,2,2-trichloro-ethyl (3RS,4RS)-4-[3-(4-benzoyloxy-butoxy)-phenyl]-3-(naphthalen-2-ylmethoxy)-piperidine-1-carboxylate as a colorless solid; R_{f}: 0.85 (SiO₂, methylene chloride:ethyl acetate=95:5)

### Example 61

The following compounds were obtained in analogy to the procedure described in Example 10(b) by cleavage of the BOC group using zinc bromide in methylene chloride:

1)—(3RS,4RS)-2-[4-[3-(Naphthalen-2-yloxy)-piperidin-4-yl]-phenoxy]-1-phenyl-ethanone hydrobromide as a colorless solid, MS: 452 (M+H)⁺, from tert-butyl (3RS,4RS)-3-(naphthalen-2-yl-methoxy)-4-[4-(2-oxo-2-phenyl-ethoxy)-phenyl]-piperidine-1-carboxylate;

2)—(3RS,4RS)-2-[4-[3-(2-methoxy-benzyloxy)-piperidin-4-yl]-phenoxy]-ethyl benzoate hydrobromide as a beige colored solid, MS: 462 (M+H)⁺, from tert-butyl (3RS,4RS)-4-[4-(2-benzoyloxy-ethoxy)-phenyl]-3-(2-methoxy-benzyloxy)-piperidine-1-carboxylate;

3)—(3RS,4RS)-4-(4-[1,3]dioxolan-2-ylmethoxy-phenyl)-3-(naphthalen-2-ylmethoxy)-piperidine as a yellowish solid, MS: 420 (M+H)⁺, from tert-butyl (3RS,4RS)-4-[4-([1,3]dioxolan-2-yl-methoxy)-phenyl]-3-(naphthalen-2-ylmethoxy)-piperidine-1-carboxylate.

The BOC derivatives used as the starting materials were obtained as follows:

(a) In an analogous manner to the procedure described in Example 14, by alkylating tert-butyl (3RS,4RS)-3-hydroxy-4-(4-hydroxy-phenyl)-piperidine-1-carboxylate with allyl bromide in the presence of potassium carbonate there was obtained tert-butyl (3RS,4RS)-4-(4-allyloxy-phenyl)-3-hydroxy-piperidine-1-carboxylate as a colorless solid; m.p.: 113° C. (hexane).

(b) In analogy to the procedure described in Example 3(c) by alkylating tert-butyl (3RS,4RS)-4-(4-allyloxy-phenyl)-3-hydroxy-piperidine-1-carboxylate with 2-bromomethylnaphthalene there was obtained tert-butyl (3RS,4RS)-4-(4-allyloxy-phenyl)-3-(naphthalen-2-ylmethoxy)-piperidine-1-carboxylate as a colorless oil; MS: 474 (M+H)⁺.

(c) A mixture of 400 mg (0.8 mmol) of tert-butyl (3RS,4RS)-4-(4-allyloxy-phenyl)-3-(naphthalen-2-ylmethoxy)-piperidine-1-carboxylate, 0.2 ml of triethylamine, 0.5 ml of water and 78 mg of tris-(triphenylphosphine)rhodium(I) chloride in 10 ml of ethanol was stirred at reflux for 1 hour. The tert-butyl (3RS,4RS)-4-(4-hydroxy-phenyl)-3-(naphthalen-2-ylmethoxy)-piperidine-1-carboxylate formed in part (30% of theory), MS: 434 (M+H)⁺, was separated by chromatography and was used in the following step.

(d) In an analogous manner to the procedure described in Example 14, by alkylating tert-butyl (3RS,4RS)-4-(4-hydroxy-phenyl)-3-(naphthalen-2-ylmethoxy)-piperidine-1-carboxylate with phenacyl bromide in the presence of potassium carbonate there was obtained tert-butyl (3RS,4RS)-3-(naphthalen-2-ylmethoxy)-4-[4-(2-oxo-2-phenyl-ethoxy)-phenyl]-piperidine-1-carboxylate as a yellowish solid; MS: 551 (M)⁺.

(e) In analogy to the procedure described in Example 3(c), by alkylating a mixture of tert-butyl (3RS,4RS)-3-hydroxy-4-[4-[2-[(RS)- and -[(SR)-tetrahydro-pyran-2-yloxy)-ethoxy]-phenyl]-piperidine-1-carboxylate [Example 53(a)] with 2-methoxybenzyl chloride there was obtained tert-butyl (3RS,4RS)-3-(2-methoxy-benzyloxy)-4-{4-[2-[(RS)- and [(SR)-tetrahydro-pyran-2-yloxy]-ethoxy]-phenyl}-piperidine-1-carboxylate as a colorless oil.

(f) In analogy to the procedure described in Example 53(c), by cleaving the THP ether from tert-butyl (3RS,4RS)-3-(2-methoxy-benzyloxy)-4-{4-[2-[(RS)- and [(SR)-tetrahydro-pyran-2-yloxy]-ethoxy]-phenyl}-piperidine-1-carboxylate there was obtained tert-butyl (3RS,4RS)-4-[4-(2-hydroxy-ethoxy)-phenyl]-3-(2-methoxy-benzyloxy)-piperidine-1-carboxylate.

(g) In analogy to the procedure described in Example 22(k), by acylating tert-butyl (3RS,4RS)-4-[4-(2-hydroxy-ethoxy)-phenyl]-3-(2-methoxy-benzyloxy)-piperidine-1-carboxylate there was obtained tert-butyl (3RS,4RS)-4-[4-(2-benzoyloxy-ethoxy)-phenyl]-3-(2-methoxy-benzyloxy)-piperidine-1-carboxylate as a colorless, viscous liquid; MS: 562 (M+H)⁺.

(h) In an analogous manner to the procedure described in Example 14, by alkylating tert-butyl (3RS,4RS)-3-hydroxy-4-(4-hydroxy-phenyl)-piperidine-1-carboxylate with 2-bromomethyl-1,3-dioxolane in the presence of potassium carbonate there was obtained tert-butyl (3RS,4RS)-4-[4-([1,3]dioxolan-2-ylmethoxy)-phenyl]-3-hydroxy-piperidine-1-carboxylate as a colorless solid; m.p.: 136-137° C. (hexane).

(i) In analogy to the procedure described in Example 3(c), by alkylating tert-butyl (3RS,4RS)-4-[4-([1,3]dioxolan-2-ylmethoxy)-phenyl]-3-hydroxy-piperidine-1-carboxylate with 2-bromomethylnaphthalene there was obtained tert-butyl (3RS,4RS)-4-[4-([1,3]dioxolan-2-ylmethoxy)-phenyl]-3-(naphthalen-2-ylmethoxy)-piperidine-1-carboxylate as a colorless solid; MS: 520 (M+H)⁺.

### Example 62

A solution of 210 mg (0.425 mmol) of tert-butyl (3RS,4RS,5SR)-4-(4-chloro-phenyl)-3-naphthalen-2-ylmethoxy-5-propyl-piperidine-1-carboxylate in 18 ml of methanol was treated with 12 ml of 1 N hydrochloric acid and stirred at 50° C. overnight. Subsequently, the solution was evaporated under reduced pressure, the residue was taken up in warm toluene and again evaporated, the product beginning to separate out. There were obtained 153 mg (84% of theory) of (3RS,4RS,5SR)-4-(4-chloro-phenyl)-3-naphthalen-2-ylmethoxy-5-propyl-piperidine as a colorless solid. MS: 252 (M-naphthylmethyl)⁺.

The tert-butyl (3RS,4RS,5SR)-4-(4-chloro-phenyl)-3-naphthalen-2-ylmethoxy-5-propyl-piperidine-1-carboxylate used as the starting material was prepared as follows:

(a) In an analogous manner to the procedure described by A. Ziering et al. in J. Org. Chem. 22, 1521-1528 (1957) for the preparation of piperidin-4-ones from the corresponding acrylic acid esters by reaction with methylamine or benzylamine, reaction with ethyl acrylate or methyl acrylate, cyclization and finally decarboxylation, starting from ethyl 2-propyl-acrylate and methylamine there was obtained (3RS)-1-methyl-3-propyl-piperidin-4-one as a colorless oil; R_{f}: 0.38 (SiO₂, methylene chloride:methanol=95:5).

(b) 83.8 ml (134 mmol) of n-BuLi (1.6 N in hexane) were added dropwise within 30 minutes to a solution, cooled to −78° C., of 25.68 g (134 mmol) of 1-bromo-4-chloro-benzene in 250 ml of tert-butyl methyl ether. After completion of the addition the mixture was stirred at −78° C. for 1 hour. Thereafter, a solution of 10.41 g (67.05 mmol) of (3RS)-1-methyl-3-propyl-piperidin-4-one in 100 ml of tert-butyl methyl ether was added dropwise at −70 to −65° C. After the dropwise addition the mixture was stirred at −78° C. for 2 hours. For the working-up, the reaction mixture was poured on to ice, transferred to a separating funnel and the organic phase was separated. The aqueous phase was extracted with ethyl acetate and subsequently the combined organic phases were dried over sodium sulfate and evaporated under reduced pressure. For purification, the crude product was chromatographed on silica gel using a 95:5:0.1 mixture of methylene chloride, methanol and ammonia as the eluent. There were obtained 15.9 g (88% of theory) of (3RS,4RS)- and (3RS,4SR)-4-(4-chloro-phenyl)-1-methyl-3-propyl-piperidin-4-ol as a colorless solid; MS: 267 (M)⁺.

(c) A solution of 13.68 g (51.06 mmol) of (3RS,4RS)- and (3RS,4SR)-4-(4-chloro-phenyl)-1-methyl-3-propyl-piperidin-4-ol in 67 ml of trifluoroacetic acid was boiled under reflux for 18 hours. Thereafter, the reaction solution was evaporated under reduced pressure. The residue was partitioned between a saturated sodium carbonate solution and ether, the separated aqueous phase was back-extracted with ether and finally the combined organic phases were dried over sodium sulfate and evaporated under reduced pressure. For purification of the crude product and separation of isomeric olefins, [the residue] was chromatographed on silica gel using a 96:4 mixture of methylene chloride and methanol as the eluent. There were obtained 9.71 g (76% of theory) of (RS)-4-(4-chloro-phenyl)-1-methyl-3-propyl-1,2,3,6-tetrahydro-pyridine, MS: 249 (M)⁺, and 1.74 g of 4-(4-chloro-phenyl)-1-methyl-5-propyl-1,2,3,6-tetrahydro-pyridine, MS: 248 (M-H)⁺, each as a yellowish oil.

(d) 2.23 g (60 mmol) of sodium borohydride were added spatula-wise to a suspension of 9.7 g (39 mmol) of (RS)-4-(4-chloro-phenyl)-1-methyl-3-propyl-1,2,3,6-tetrahydro-pyridine in 80 ml of 1,2-dimethoxyethane in such a manner that the temperature did not rise above 35° C. Subsequently, 13.2 ml of boron trifluoride etherate dissolved in 15 ml of 1,2-dimethoxyethane were added dropwise during 45 minutes and thereafter the reaction mixture was stirred at room temperature for 2 hours. Subsequently, firstly a solution of 15.65 g (277 mmol) of potassium hydroxide dissolved in 60 ml of water was slowly added dropwise at about 30° C. and thereafter within 15 minutes 11.2 ml of a 30% hydrogen peroxide solution was added, with the temperature rising to 40° C. Subsequently, the mixture was boiled under reflux for 2.5 hours. For the working-up, the cooled reaction mixture was filtered over Dicalit and this was rinsed with ethyl acetate. The solution obtained was treated with 100 ml of ethyl acetate and 100 ml of water, the organic phase was separated and then the aqueous phase was back-extracted with 100 ml of ethyl acetate. The combined organic phases were dried over sodium sulfate and evaporated under reduced pressure. For purification, the crude product was chromatographed on silica gel using a 95:5 mixture of methylene chloride and methanol as the eluent. There were obtained 8.64 g (73% of theory) of (3RS,4SR,5RS)-4-(4-chloro-phenyl)-1-methyl-5-propyl-piperidin-3-ol as a colorless solid; MS: 267 (M)⁺.

(e) A mixture of 7.19 g (26.85 mmol) of (3RS,4SR,5RS)-4-(4-chloro-phenyl)-1-methyl-5-propyl-piperidin-3-ol, 5.96 g (80.7 mmol) of lithium carbonate and 14.22 g (67.1 mmol) of 2,2,2-trichloroethyl chloroformate in 200 ml of toluene was stirred at 105° C. for 8 hours. For the working-up, the cooled reaction mixture was treated with aqueous sodium carbonate solution and ethyl acetate. The organic phase was separated, dried over sodium sulfate and evaporated under reduced pressure. For purification, the crude product was chromatographed on silica gel using a 1:1 mixture of methylene chloride and hexane as the eluent. There were obtained 13.05 g (80% of theory) of 2,2,2-trichloro-ethyl (3RS,4SR,5SR)-4-(4-chloro-phenyl)-3-propyl-5-(2,2,2-trichloroethoxycarbonyloxy)-piperidine-1-carboxylate as a yellowish oil; MS: 621 (M+NH₄)⁺.

(f) A mixture of 13.05 g (21.6 mmol) of 2,2,2-trichloro-ethyl (3RS,4SR,5SR)-4-(4-chloro-phenyl)-3-propyl-5-(2,2,2-trichloroethoxycarbonyloxy)-piperidine-1-carboxylate and 14.5 g of zinc in 200 ml of glacial acetic acid was treated in an ultrasound bath for 15 hours. In order to complete the reaction, a further 5 g of zinc were subsequently added and the mixture was left in the ultrasound bath for a further 5 hours. For the working-up, the zinc was filtered off under suction, the residue was rinsed with glacial acetic acid and the solution was evaporated to dryness under reduced pressure. The residue was partitioned between 1 N NaOH and ethyl acetate, the separated aqueous phase was then again extracted with ethyl acetate and finally the combined organic phases were evaporated under reduced pressure. The crude product obtained was crystallized from diethyl ether and gave 3.0 g (55% of theory) of (3RS,4RS,5SR)-4-(4-chloro-phenyl)-5-propyl-piperidin-3-ol as colorless crystals; MS: 253 (M)⁺. For purification, the mother liquor was chromatographed on silica gel using a 90:10:0.1 mixture of methylene chloride, methanol and ammonia as the eluent. There were obtained a further 1.1 g (20% of theory) of (3RS,4RS,5SR)-4-(4-chloro-phenyl)-5-propyl-piperidin-3-ol.

(g) A solution of 3.05 g (12.0 mmol) of (3RS,4RS,5SR)-4-(4-chloro-phenyl)-5-propyl-piperidin-3-ol in 20 ml of dimethyl-formamide was treated at 0° C. with 1.34 g (13.2 mmol) of triethylamine and 3.02 g (13.8 mmol) of di-tert-butyl dicarbonate and the mixture was stirred at room temperature for 15 hours. Subsequently, the dimethylformamide was distilled off in an oil pump vacuum and, for purification, the residue was chromatographed on silica gel using a 99:1 mixture of methylene chloride and methanol as the eluent. There were obtained 3.92 g (92% of theory) of tert-butyl (3RS,4RS,5SR)-4-(4-chloro-phenyl)-3-hydroxy-5-propyl-piperidine-1-carboxylate as a colorless solid; MS: 297 (M-C₄H₈)⁺.

(h) 37 mg (0.85 mmol) of sodium hydride (55% dispersion in refined oil) were added to a solution of 200 mg (0.56 mmol) of tert-butyl (3RS,4RS,5SR)-4-(4-chloro-phenyl)-3-hydroxy-5-propyl-piperidine-1-carboxylate and 188 mg (0.85 mmol) of 2-bromomethylnaphthalene in 10 ml of dimethylformamide and the reaction mixture was stirred at room temperature for 5 hours. For the working-up, the reaction mixture was evaporated in an oil pump vacuum, the residue was partitioned between water and ether and thereafter the separated aqueous phase was extracted five times with 50 ml of ether each time. The combined ether extracts were washed with saturated sodium chloride solution, dried over sodium sulfate and evaporated under reduced pressure. For purification, the crude product was chromatographed on silica gel using a 96:4 mixture of toluene and ethyl acetate as the eluent. There were obtained 215 mg (77% of theory) of tert-butyl (3RS,4RS,5SR)-4-(4-chloro-phenyl)-3-naphthalen-2-ylmethoxy-5-propyl-piperidine-1-carboxylate as a colorless oil; MS: 494 (M+H)⁺.

### Example 63

The following compounds were obtained in an analogous manner to that described in Example 62:

1)—(3RS,4RS,5SR)-4-(4-Chloro-phenyl)-3-(4-methoxy-benzyloxy)-5-propyl-piperidine as a yellowish oil, MS: 252 (M-methoxybenzyl)⁺, from tert-butyl (3RS,4RS,5SR)-4-(4-chloro-phenyl)-3-(4-methoxy-benzyloxy)-5-propyl-piperidine-1-carboxylate;

2)—(3RS,4RS,5SR)-4-(4-chloro-phenyl)-3-(1-ethyl-1H-benzimidazol-2-ylmethoxy)-5-propyl-piperidine hydrochloride as a colorless solid, MS: 412 (M+H)⁺, from tert-butyl (3RS,4RS,5SR)-4-(4-chloro-phenyl)-3-(1-ethyl-1H-benzimidazol-2-ylmethoxy)-5-propyl-piperidine-1-carboxylate.

The BOC derivatives used as the starting materials were obtained as follows:

In an analogous manner to that described in Example 62(h), by alkylating tert-butyl (3RS,4RS,5SR)-4-(4-chloro-phenyl)-3-hydroxy-5-propyl-piperidine-1-carboxylate with 4-methoxybenzyl chloride there was obtained tert-butyl (3RS,4RS,5SR)-4-(4-chloro-phenyl)-3-(4-methoxy-benzyloxy)-5-propyl-piperidine-1-carboxylate as a colorless solid, MS: 416 (M-C₄H₉)⁺.

In an analogous manner to that described in Example 62(h), by alkylating tert-butyl (3RS,4RS,5SR)-4-(4-chloro-phenyl)-3-hydroxy-5-propyl-piperidine-1-carboxylate with 2-chloromethyl-1-ethyl-1H-benzoimidazole [Acta Pol. Pharm. 1977, 34(4), 359-369] there was obtained tert-butyl (3RS,4RS,5SR)-4-(4-chloro-phenyl)-3-(1-ethyl-1H-benzimidazol-2-ylmethoxy)-5-propyl-piperidine-1-carboxylate as a colorless oil, MS: 495 (M+H)⁺.

### Example 64

In an analogous manner to that described in Example 5, by treating 2-trimethylsilanylethyl (3RS,4RS,5SR)-3-(4-benzyloxy-naphthalen-2-ylmethoxy)-4-(4-fluoro-phenyl)-5-propyl-piperidine-1-carboxylate with tetrabutylammonium fluoride solution there was obtained (3RS,4RS,5SR)-3-(4-benzyloxy-naphthalen-2-ylmethoxy)-4-(4-fluoro-phenyl)-5-propyl-piperidine as an almost colorless solid, MS: 484 (M+H)⁺.

The 2-trimethylsilanylethyl (3RS,4RS,5SR)-3-(4-benzyloxy-naphthalen-2-ylmethoxy)-4-s (4-fluoro-phenyl)-5-propyl-piperidine-1-carboxylate used as the starting substance was obtained as follows:

(a) In an analogous manner to that described in Example 62(a)-(d), starting from (RS)-1-benzyl-3-propyl-piperidin-4-one, MS: 231 (M)⁺, and 1-bromo-4-fluorobenzene there was obtained (3RS,4RS,5SR)-1-benzyl-4-(4-fluoro-phenyl)-5-propyl-piperidin-3-ol as a colorless solid, MS: 327 (M)⁺.

(b) In an analogous manner to that described in Example 62(h), by alkylating (3RS,4RS,5SR)-1-benzyl-4-(4-fluoro-phenyl)-5-propyl-piperidin-3-ol with 1-benzyloxy-3-chloromethyl-naphthalene there was obtained (3RS,4RS,5SR)-1-benzyl-3-(4-benzyloxy-naphthalen-2-ylmethoxy)-4-(4-fluoro-phenyl)-5-propyl-piperidine as a yellow resin, MS (ISP): 574 (M+H)⁺.

(c) In an analogous manner to that described in Example 1 (d), by reacting (3RS,4RS,5SR)-1-benzyl-3-(4-benzyloxy-naphthalen-2-ylmethoxy)-4-(4-fluoro-phenyl)-5-propyl-piperidine with β-trimethylsilylethyl chloroformate [Synthesis 346 (1987)] there was obtained 2-trimethylsilanylethyl (3RS,4RS,5SR)-3-(4-benzyloxy-naphthalen-2-ylmethoxy)-4-(4-fluoro-phenyl)-5-propyl-piperidine-1-carboxylate as a pale yellow syrup, MS: 628 (M+H)⁺.

The 1-benzyloxy-3-chloromethyl-naphthalene used as the starting material was obtained as follows:

(a) In an analogous manner to that described in Example 14(a), by alkylating ethyl 4-hydroxy-naphthalene-2-carboxylate [J. Agric. Chem Soc. Japan 24, 313 (1950)] with benzyl bromide there was obtained ethyl 4-benzyloxy-naphthalene-2-carboxylate as an almost colorless solid, R_{f}: 0.53 (SiO₂, hexane:ethyl acetate=4:1).

(b) Reduction of ethyl 4-benzyloxy-naphthalene-2-carboxylate analogously to Example 7 (b) yielded (4-benzyloxy-naphthalen-2-yl)-methanol as a colorless solid, R_{f}: 0.42 (SiO₂, hexane:ethyl acetate=2:1).

(c) Chlorination of (4-benzyloxy-naphthalen-2-yl)-methanol analogously to Example 7(c) yielded 1-benzyloxy-3-chloromethyl-naphthalene as a colorless solid, MS: 282 (M)⁺.

### Example 65

The following compounds were prepared in an analogous manner to that described in Example 62:

1)—(3RS,4RS,5SR)-4-(4-Chloro-phenyl)-5-isopropyl-3-(naphthalen-2-ylmethoxy)-piperidine hydrochloride as a colorless solid, MS: 394 (M+H)⁺, from tert-butyl (3RS,4RS,5SR)-4-(4-chloro-phenyl)-5-isopropyl-3-naphthalen-2-ylmethoxy-piperidine-1-carboxylate;

2)—(3RS,4RS,5SR)-4-(4-bromo-phenyl)-5-isobutyl-3-naphthalen-2-ylmethoxy-piperidine hydrochloride as a colorless solid, MS: 310 (M-naphthylmethyl)⁺, from tert-butyl (3RS,4RS,5SR)-4-(4-bromo-phenyl)-5-isobutyl-3-naphthalen-2-ylmethoxy-piperidine-1-carboxylate;

3)—(3RS,4RS,5SR)-4-(4-fluoro-phenyl)-5-methyl-3-naphthalen-2-ylmethoxy-piperidine hydrochloride as a colorless solid, R_{f}: 0.37 (SiO₂, methylene chloride:methanol:ammonia=95:5:0.1), from tert-butyl (3RS,4RS,5SR)-4-(4-fluoro-phenyl)-5-methyl-3-naphthalen-2-ylmethoxy-piperidine-1-carboxylate;

4)—(3RS,4RS,5SR)-5-benzyl-4-(4-fluoro-phenyl)-3-naphthalen-2-ylmethoxy-piperidine hydrochloride as a colorless solid, MS: 426 (M+H)⁺, from tert-butyl (3RS,4RS,5SR)-5-benzyl-4-(4-fluorophenyl)-3-(naphthalen-2-ylmethoxy)-piperidine-1-carboxylate.

The BOC derivatives used as the starting materials were obtained as follows:

The following procedure was carried out in an analogous manner to that described in Example 62(a)-(h):

(a) Starting from ethyl 2-isopropyl-acrylate and methylamine there was obtained (3RS)-1-methyl-3-isopropyl-piperidin-4-one as a colorless oil; MS: 155 (M)⁺. By reaction with 1-bromo-4-chlorobenzene there was obtained (3RS,4RS)- and (3RS,4SR)-4-(4-chloro-phenyl)-3-isopropyl-1-methyl-piperidin-4-ol as a colorless solid; MS: 267 (M)⁺. Following elimination using trifluoroacetic acid and subsequent chromatographic separation gave the two isomeric olefins, (RS)-4-(4-chloro-phenyl)-3-isopropyl-1-methyl-1,2,3,6-tetrahydro-pyridine, MS: 249 (M)⁺, and 4-(4-chloro-phenyl)-5-isopropyl-1-methyl-1,2,3,6-tetrahydro-pyridine, each as a colorless oil. Hydroboration of (RS)-4-(4-chloro-phenyl)-3-isopropyl-1-methyl-1,2,3,6-tetrahydro-pyridine gave (3RS,4RS,5SR)-4-(4-chloro-phenyl)-5-isopropyl-1-methyl-piperidin-3-ol as a colorless solid; MS: 267 (M)⁺. Further reaction with 2,2,2-trichloroethyl chloroformate yielded 2,2,2-trichloroethyl (3SR,4RS,5RS)-4-(4-chloro-phenyl)-3-isopropyl-5-(2,2,2-trichloro-ethoxycarbonyloxy)-piperidine-1-carboxylate as a yellowish oil; MS: 619, 621, 623, 625 (M+NH₄)⁺. Cleavage of the TROC group with zinc in glacial acetic acid gave (3RS,4RS,5SR)-4-(4-chloro-phenyl)-5-isopropyl-piperidin-3-ol as a colorless solid: MS: 253 (M)⁺. By introduction of the BOC group there was obtained therefrom tert-butyl (3RS,4RS,5SR)-4-(4-chloro-phenyl)-3-hydroxy-5-isopropyl-piperidine-1-carboxylate as a colorless solid; MS: 297 (M-C₄H₈)⁺. Finally, alkylation with 2-bromomethylnaphthalene yielded tert-butyl (3RS,4RS,5SR)-4-(4-chloro-phenyl)-5-isopropyl-3-naphthalen-2-ylmethoxy-piperidine-1-carboxylate as a colorless solid; MS: 437 (M-C₄H₈)⁺.

The following procedure was carried out in an analogous manner to that described in Example 62(a)-(h):

(b) Starting from methyl 2-isobutyl-acrylate and benzylamine there was obtained (3RS)-1-benzyl-3-isobutyl-piperidin-4-one as a yellowish oil; MS: 245 (M)⁺. By reaction with 1,4-dibromobenzene and subsequent elimination using trifluoroacetic acid as well as chromatographic separation there were obtained the two isomeric olefins, (RS)-1-benzyl-4-(4-bromo-phenyl)-3-isobutyl-1,2,3,6-tetrahydro-pyridine, MS: 383 (M)⁺, and 1-benzyl-4-(4-bromo-phenyl)-5-isobutyl-1,2,3,6-tetrahydro-pyridine, each as a brownish oil. Subsequent hydroboration of (RS)-1-benzyl-4-(4-bromo-phenyl)-3-isobutyl-1,2,3,6-tetrahydro-pyridine gave (3RS,4RS,5SR)-1-benzyl-4-(4-bromo-phenyl)-5-isobutyl-piperidin-3-ol as a colorless solid; MS: 401 (M)⁺. Further reaction with 2,2,2-trichloroethyl chloroformate yielded 2,2,2-trichloro-ethyl (3RS,4R,5SR)-4-(4-bromo-phenyl-5-isobutyl-3-(2,2,2-trichloroethoxycarbonyloxy)-piperidine-1-carboxylate as a colorless solid; R_{f}: 0.25 (SiO₂, methylene chloride:hexane=1:1). Cleavage of the TROC group with zinc in glacial acetic acid gave (3RS,4RS,5SR)-4-(4-bromo-phenyl)-5-isobutyl-piperidine-3-ol as a colorless solid: MS: 311 (M)⁺. By introduction of the BOC group there was obtained therefrom tert-butyl (3RS,4RS,5SR)-4-(4-bromo-phenyl)-3-hydroxy-5-isobutyl-piperidine-1-carboxy late as a colorless solid; MS: 412 (M+H)⁺. Finally, alkylation with 2-bromomethylnaphthalene yielded tert-butyl (3RS,4RS,5SR)-4-(4-bromo-phenyl)-5-isobutyl-3-naphthalen-2-ylmethoxy-piperidine-1-carboxylate as a colorless foam; MS: 552 (M+H)⁺.

The following procedure was carried out in an analogous manner to that described in Example 62(b)-(d):

(c) From (RS)-1-benzyl-3-methyl-piperidin-4-one and 1-bromo-4-fluorobenzene there was obtained (3RS,4RS)- and (3RS,4SR)-1-benzyl-4-(4-fluoro-phenyl)-3-methyl-piperidine-4-ol as a colorless solid; MS: 299 (M)⁺. By elimination using trifluoroacetic acid and subsequent chromatographic separation there were obtained the two isomeric olefins, (RS)-1-benzyl-4-(4-fluoro-phenyl)-3-methyl-1,2,3,6-tetrahydro-pyridine, MS: 281 (M)⁺, and 1-benzyl-4-(4-fluoro-phenyl)-5-methyl-1,2,3,6-tetrahydro-pyridine, MS: 281 (M)⁺, each as a brownish oil. Subsequent hydroboration of the (RS)-1-benzyl-4-(4-fluoro-phenyl)-3-methyl-1,2,3,6-tetrahydro-pyridine gave (3RS,4RS,5SR)-1-benzyl-4-(4-fluoro-phenyl)-5-methyl-piperidine-3-ol as a colorless solid; MS: 299 (M)⁺.

(d) A solution of 600 mg (2 mmol) of (3RS,4RS,55R)-1-benzyl-4-(4-fluoro-phenyl)-5-methyl-piperidin-3-ol in 20 ml of methanol was hydrogenated with 60 mg of palladium/charcoal (10%) at room temperature under normal pressure. For the working-up, the catalyst was filtered off, stirred in warm methanol and again filtered off. The combined methanol solutions were evaporated under reduced pressure. The resulting crude product (410 mg) was used without further purification in the following step. For analytical purposes, a sample was recrystallized from ether/hexane. The (3RS,4RS,5SR)-4-(4-fluoro-phenyl)-5-methyl-piperidin-3-ol hydrochloride was obtained in the form of colorless crystals; MS: 209 (M)⁺.

(e) From (3RS,4RS,5SR)-4-(4-fluoro-phenyl)-5-methyl-piperidin-3-ol hydrochloride by introduction of the BOC protecting group there was obtained tert-butyl (3RS,4RS,5SR)-4-(4-fluoro-phenyl)-3-hydroxy-5-methyl-piperidine-1-carboxylate as a colorless oil; MS: 253 (M-C₄H₈)⁺. Finally, alkylation with 2-bromomethylnaphthalene yielded tert-butyl (3RS,4RS,5SR)-4-(4-fluoro-phenyl)-5-methyl-3-naphthalen-2-ylmethoxy-piperidine-1-carboxylate as a colorless solid; MS: 393 (M-C₄H₈)⁺.

The following procedure was carried out in an analogous manner to that described in Example 62(a)-(d) and (g)-(h):

(a) Starting from ethyl 2-benzyl-acrylate and benzylamine there was obtained (3RS)-1,3-dibenzyl-piperidin-4-one as a yellowish oil; MS: 279 (M)⁺. By reaction with 1-bromo-4-fluorobenzene there was obtained (3RS,4RS)- and (3RS,4SR)-1,3 dibenzyl-4-(4-fluoro-phenyl)-piperidin-4-ol as a colorless solid; MS: 375 (M)⁺. By elimination using trifluoroacetic acid and subsequent chromatographic separation there were obtained the two isomeric olefins, (RS)-1,3-dibenzyl-4-(4-fluoro-phenyl)-1,2,3,6-tetrahydropyridine as a yellow solid, MS: 357 (M)⁺, and 1,5-dibenzyl-4-(4-fluoro-phenyl)-1,2,3,6-tetrahydro-pyridine. Subsequent hydroboration of the (RS)-1,3-dibenzyl-4-(4-fluoro-phenyl)-1,2,3,6-tetrahydro-pyridine gave (3RS,4RS,5SR)-1,5-dibenzyl-4-(4-fluoro-phenyl)-piperidin-3-ol as a colorless solid; MS: 375 (M)⁺. Cleavage of the benzyl group was effected using catalytic hydrogenation in an analogous manner to that described in the above Example and yielded (3RS,4RS,5SR)-5-benzyl-4-(4-fluoro-phenyl)-piperidin-3-ol as a colorless solid; MS: 285 (M)⁺. By introduction of the BOC group there was obtained therefrom tert-butyl (3RS,4SR,5SR)-3-benzyl-4-(4-fluoro-phenyl)-5-hydroxy-piperidine-1-carboxylate as a colorless solid; MS: 329 (M-C₄H₈)⁺. Finally, alkylation with 2-bromomethylnaphthalene yielded tert-butyl (3RS,4RS,5SR)-5-benzyl-4-(4-fluoro-phenyl)-3-(naphthalen-2-ylmethoxy)-piperidine-1-carboxylate as a colorless foam; R_{f}: 0.32 (SiO₂, toluene:ethyl acetate=95:5).

### Example 66

40 mg (0.08 mmol) of tert-butyl (3SR,4RS,5RS)-4-(4-chloro-phenyl)-3-(1-ethyl-propyl)-5-(naphthalen-2-ylmethoxy)-piperidine-carboxylate were dissolved in 5 ml of dry methylene chloride and treated with 35 mg (0.16 mmol) of anhydrous zinc bromide in an analogous manner to the procedure described by A. Mann et al. in Synth. Comm. 19(18), 3139-3142 (1989). The reaction mixture was stirred at room temperature for 5 hours. For the working-up, the reaction mixture was evaporated under reduced pressure and the crude product obtained was purified by chromatography on silica gel using a 90:10:0.1 mixture of methylene chloride, methanol and ammonia as the eluent. There were obtained 28 mg (86% of theory) of (3SR,4RS,5RS)-4-(4-chloro-phenyl)-3-(1-ethyl-propyl)-5-(naphthalen-2-ylmethoxy)-piperidine as a colorless oil; MS: 422 (M+H)⁺.

The tert-butyl (3SR,4RS,5RS)-4-(4-chloro-phenyl)-3-(1-ethyl-propyl)-5-(naphthalen-2-ylmethoxy)-piperidine-carboxylate used as the starting material was obtained as follows in an analogous manner to that described in Example 62(a)-(h):

Starting from methyl 2-(1-ethyl-propyl)-acrylate and methylamine there was obtained (3RS)-3-(1-ethyl-propyl)-1-methyl-piperidin-4-one as a colorless oil; MS: 183 (M)⁺. By reaction with 1-bromo-4-chlorobenzene there was obtained therefrom (3RS,4RS)- and (3RS,4SR)-4-(4-chloro-phenyl)-3-(1-ethyl-propyl)-1-methyl-piperidin-4-ol as a colorless solid; MS: 295 (M)⁺. Subsequent elimination using trifluoroacetic acid and chromatographic separation gave the two isomeric olefins, (RS)-4-(4-chloro-phenyl)-3-(1-ethyl-propyl)-1-methyl-1,2,3,6-tetrahydro-pyridine, MS: 277 (M)⁺, and 4-(4-chloro-phenyl)-5-(1-ethyl-propyl)-1-methyl-1,2,3,6-tetrahydro-pyridine, each as a colorless oil. Subsequent hydroboration of the (RS)-4-(4-chloro-phenyl)-3-(1-ethyl-propyl)-1-methyl 1,2,3,6-tetrahydro-pyridine gave (3RS,4RS,5SR)-4-(4-chloro-phenyl)-5-(1-ethyl-propyl)-1-methyl-piperidin-3-ol as a colorless solid; MS: 296 (M+H)⁺. Further reaction with 2,2,2-trichloroethyl chloroformate yielded 2,2,2-trichloro-ethyl (3SR,4RS,5RS)-4-(4-chloro-phenyl)-3-(1-ethyl-propyl)-5-(2,2,2-trichloro-ethoxycarbonyloxy)-piperidine-1-carboxylate as a colorless oil; MS: 653 (M+Na)⁺. Cleavage of the TROC group with zinc in glacial acetic acid gave (3RS,4RS,5SR)-4-(4-chloro-phenyl)-5-(1-ethyl-propyl)-piperidin-3-ol as a colorless solid: MS: 281 (M)⁺. By introduction of the BOC protecting group there was obtained therefrom tert-butyl (3SR,4RS,5RS)-4-(4-chloro-phenyl)-3-(1-ethyl-propyl)-5-hydroxy-piperidine-1-carboxylate as a colorless oil; MS: 325 (M-C₄H₈)⁺. Finally, alkylation with 2-bromomethylnaphthalene yielded tert-butyl (3SR,4RS,5RS)-4-(4-chloro-phenyl)-3-(1-ethyl-propyl)-5-(naphthalen-2-ylmethoxy)-piperidine-carboxylate; R_{f}: 0.41 (SiO₂, toluene:ethyl acetate=95:5).

### Example 67

In an analogous manner to that described in Example 66, from tert-butyl (3SR,4RS,5RS)-4-(4-chloro-phenyl)-3-isopropyl-5-(4-methoxy-benzyloxy)-piperidine-carboxylate there was obtained (3SR,4RS,5RS)-4-(4-chloro-phenyl)-3-isopropyl-5-(4-methoxy-benzyloxy)-piperidine as a colorless oil; R_{f}: 0.21 (SiO₂, methylene chloride:methanol:ammonia=95:5:0.1.

The tert-butyl (3SR,4RS,5RS)-4-(4-chloro-phenyl)-3-isopropyl-5-(4-methoxy-benzyloxy)-piperidine-carboxylate used as the starting material was obtained in an analogous manner to that described in Example 62(h) by alkylating tert-butyl (3RS,4RS,5SR)-4-(4-chloro-phenyl)-3-hydroxy-5-isopropyl-piperidine-1-carboxylate (Example 65) with 4-methoxy-benzyl chloride; R_{f}: 0.39 (SiO₂, toluene:ethyl acetate=9:1).

### Example 68

A solution of 60 mg (0.11 mg) of tert-butyl (3RS,4RS,5SR)-4-(4-bromo-phenyl)-5-methoxymethyl-3-naphthalen-2-yl-methoxy-piperidine-1-carboxylate in 5 ml of methylene chloride was treated with 2 ml of 2 N hydrogen chloride in methanol and stirred at room temperature for 2 hours. For the working-up, the reaction solution was evaporated under reduced pressure. The residue was recrystallized from diethyl ether and gave 53 mg (98% of theory) of (3RS,4RS,5SR)-4-(4-bromo-phenyl)-5-methoxymethyl-3-naphthalen-2-yl-methoxy-piperidine hydrochloride as a colorless solid; MS: 442 (M+H)⁺.

The tert-butyl (3RS,4RS,5SR)-4-(4-bromo-phenyl)-5-methoxymethyl-3-naphthalen-2-ylmethoxy-piperidine-1-carboxylate used as the starting substance was obtained as follows:

(a) A solution of 7.45 g (33.7 mmol) of (3RS,4RS)-1-benzyl-3-hydroxymethyl-piperidin-4-ol and (3SR,4RS)-1-benzyl-3-hydroxymethyl-piperidin-4-ol [E. Jaeger and J. H. Biel, J. Org. Chem. 30(3), 740-744 (1965)], 10.89 g (39.6 mmol) of tert-butyidiphenylchlorosilane, 3.44 g (50.5 mmol) of imidazole and 0.2 g (1.6 mmol) of 4-dimethylaminopyridine in 80 ml of dimethylformamide was stirred at room temperature for 4 days in the presence of molecular sieve (4 Å). For the working-up, the molecular sieve was filtered off under suction and the solution was evaporated in an oil pump vacuum. The residue was digested four times in a mixture of ether and methylene chloride, the solutions obtained were combined, dried over sodium sulfate and evaporated under reduced pressure. For purification, the crude product was chromatographed on silica gel using a 98:2 mixture of methylene chloride and methanol as the eluent. There were obtained 8.23 g (53% of theory) of a mixture of (3RS,4RS)- and (3RS,4SR)-1-benzyl-3-(tert-butyl-diphenyl-silanyloxymethyl)-piperidin-4-ol as a colorless oil; MS: 459 (M)⁺.

(b) A solution of 2.45 g (19.33 mmol) of oxalyl chloride in 60 ml of methylene chloride was cooled to −70° C., then treated dropwise with 3.02 g (38.66 mmol) of dimethyl sulfoxide and stirred at −70° for 5 minutes. A solution of 8.08 g (17.6 mmol) of a mixture of (3RS,4RS)- and (3RS,4SR)-1-benzyl-3-(tert-butyl-diphenyl-silanyloxymethyl)-piperidin-4-ol in 15 ml of methylene chloride was added dropwise thereto, the mixture was then stirred for 15 minutes. Subsequently, 8.86 g (87.6 mmol) of triethylamine were added dropwise at −70° C. After warming the reaction mixture to room temperature (about 15 minutes) it was hydrolyzed in ice-water and thereafter extracted three times with 200 ml of methylene chloride each time. The combined organic phases were dried over sodium sulfate and evaporated under reduced pressure. For purification, the crude product was chromatographed on silica gel using methylene chloride as the eluent. There were obtained 6.5 g (81% of theory) of (3RS)-1-benzyl-3-(tert-butyl-diphenyl-silanyloxymethyl)-piperidin-4-one as a colorless oil; MS: 458 (M+H)⁺.

(c) In an analogous manner to that described in Example 62(b), from (3RS)-1-benzyl-3-(tert-butyl-diphenyl-silanyloxymethyl)-piperidin-4-one and 1,4-dibromobenzene there was obtained (3RS,4RS)- and/or (3RS,4SR)-1-benzyl-4-(4-bromo-phenyl)-3-(tert-butyl-diphenyl-silanyloxymethyl)-piperidin-4-ol as a colorless foam; MS: 616 (M+H)⁺.

(d) In an analogous manner to that described in Example 62(c), from (3RS,4RS)- and/or (3RS,4SR)-1-benzyl-4-(4-bromo-phenyl)-3-(tert-butyl-diphenyl-silanyloxymethyl)-piperidin-4-ol by eliminating the tert. alcohol and simultaneous cleavage of the silyl group using trifluoroacetic acid there was obtained (3RS)-[1-benzyl-4-(4-bromo-phenyl)-1,2,3,6-tetrahydro-pyridin-3-yl]-methanol as a colorless solid; MS: 360 (M+H)⁺.

(e) In an analogous manner to that described in Example 62(d), by hydroborating (3RS)-[1-benzyl-4-(4-bromo-phenyl)-1,2,3,6-tetrahydro-pyridin-3-yl]-methanol there was obtained a mixture of (3RS,4RS,5SR)-1-benzyl-4-(4-bromo-phenyl)-5-hydroxymethyl-piperidin-3-ol and (3RS,4RS)- and/or (3RS,4SR)-1-benzyl-4-(4-bromo-phenyl)-3-hydroxymethyl-piperidin-4-ol as a colorless foam.

(f) In an analogous manner to that described in (e), by treating the 15 above mixture with 2,2,2-trichloroethyl chloroformate there was obtained a mixture of 2,2,2-trichloro-ethyl (3RS,4RS,5SR)-4-(4-bromo-phenyl)-3-(2,2,2-trichloro-ethoxycarbonyloxy)-5-(2,2,2-trichloro-ethoxycarbonyloxymethyl)-piperidine-1-carboxylate and 2,2,2-trichloro-ethyl (3RS,4RS)- and/or (3RS,4SR)-4-(4-bromo-phenyl)-4-(2,2,2-trichloro-ethoxycarbonyloxy)-3-(2,2,2-trichloro-ethoxycarbonyloxymethyl)-piperidine-1-carboxylate as a colorless foam.

(g) In an analogous manner to that described in Example 62(f), by reacting the above mixture with zinc in glacial acetic acid there was obtained a 4:1 mixture of (3RS,4RS,5RS)-4-(4-bromo-phenyl)-5-hydroxymethyl-piperidin-3-ol and (3RS,4RS)- and/or (3RS,4SR)-4-(4-bromo-phenyl)-3-hydroxymethyl-piperidin-4-ol as a colorless foam.

(h) In an analogous manner to that described in Example 62(g), by introducing the BOC group and subsequent chromatographic separation there was obtained the mixture of tert-butyl (3RS,4RS,5SR)-4-(4-bromo-phenyl)-3-hydroxy-5-hydroxymethyl-piperidine-1-carboxylate as a colorless foam, MS: 386 (M+H)⁺, and tert-butyl (3RS,4RS)- and/or (3RS,4SR)-4-(4-bromo-phenyl)-4-hydroxy-3-hydroxymethyl-piperidine-1-carboxylate as a colorless solid, MS: 386 (M+H)⁺.

(i) A solution of 735 mg (1.91 mmol) of tert-butyl (3RS,4RS,5SR)-4-(4-bromo-phenyl)-3-hydroxy-5-hydroxymethyl-piperidine-1-carboxylate, 766 mg (2.75 mmol) of triphenylchloromethane and 324 mg (3.20 mmol) of triethylamine in 8 ml of methylene chloride was stirred at room temperature for 15 hours. For the working-up, the reaction mixture was evaporated under reduced pressure and the crude product was chromatographed directly on silica gel using methylene chloride as the eluent. There were obtained 1.01 g (84.5% of theory) of tert-butyl (3RS,4RS,5SR)-4-(4-bromo-phenyl)-3-hydroxy-5-trityloxymethyl-piperidine-1-carboxylate as a colorless foam; MS: 646 (M+NH₄)⁺.

(j) In an analogous manner to that described in Example 62(h), by alkylating tert-butyl (3RS,4RS,5SR)-4-(4-bromo-phenyl)-3-hydroxy-5-trityloxymethyl-piperidine-1-carboxylate with 2-bromomethylnaphthalene there was obtained tert-butyl (3RS,4RS,5SR)-4-(4-bromo-phenyl)-3-naphthalen-2-ylmethoxy-5-trityl-oxymethyl-piperidine-1-carboxylate as a colorless foam; MS: 785 (M+NH₄)⁺.

(k) A solution of 990 mg (1.29 mmol) of tert-butyl (3RS,4RS,5SR)-4-(4-bromo-phenyl)-3-naphthalen-2-ylmethoxy-5-trityloxymethyl-piperidine-1-carboxylate and 4 ml of 2 N hydrogen chloride/methanol in 5 ml of methylene chloride was stirred at room temperature for 45 minutes. For the working-up, the reaction solution was poured into 40 ml of saturated sodium carbonate solution and this was extracted twice with 40 ml of methylene chloride each time. The combined organic phases were dried over sodium sulfate and evaporated under reduced pressure. For purification and separation, the product obtained was chromatographed on silica gel: firstly with a 98:2 mixture of methylene chloride and methanol as the eluent. There were thus obtained 360 mg (54% of theory) of tert-butyl (3RS,4RS,5SR)-4-(4-bromo-phenyl)-5-hydroxymethyl-3-naphthalen-2-ylmethoxy-piperidine-1-carboxylate as a colorless solid; MS: 528 (M+H)⁺. Then with a 90:10:1 mixture of methylene chloride, methanol and ammonia, with (3SR,4RS,5RS)-4-(4-bromo-phenyl)-5-naphthalen-2-ylmethoxy-piperidin-3-yl]-methanol being obtained as a colorless solid; MS: 426 (M+H)⁺.

(l) In an analogous manner to that described in Example 62(h), by alkylating (3SR,4RS,5RS)-4-(4-bromo-phenyl)-5-naphthalen-2-ylmethoxy-piperidin-3-yl]-methanol with methyl iodide there was obtained tert-butyl (3RS,4RS,5SR)-4-(4-bromo-phenyl)-5-methoxymethyl-3-naphthalen-2-yl-methoxy-piperidine-1-carboxylate as a colorless solid; MS: 540 (M+H)⁺.

### Example 69

a) A solution of 32.5 g (163 mmol) of tert-butyl 4-oxo-piperidine-1-carboxylate in 200 ml of chloroform was treated with 24.0 g (168 mmol) of disodium hydrogen phosphate and cooled to 5° C. A solution of 27.9 g (175 mmol) of bromine in 75 ml of chloroform was added dropwise during 1 hour, thereafter the reaction mixture was warmed to room temperature and stirred for 18 hours. The reaction mixture was worked-up by extraction with ice-water and methylene chloride, the organic phase was dried over magnesium sulfate, filtered and the solvent was distilled off in a water-jet vacuum. The crude product (38 g) was chromatographed on silica gel with methylene chloride and ethyl acetate as the eluent. The thus-obtained product was recrystallized from ethyl acetate and n-hexane. There were obtained 19.1 g (42% of theory) of tert-butyl 3-bromo-4-oxo-piperidine-1-carboxylate as a pale yellow solid; MS: 277, 279 (M)⁺.

b) A solution of 2.78 g (10 mmol) of tert-butyl 3-bromo-4-oxo-piperidine-1-carboxylate and 2.09 g (12 mmol) of 2-mercaptomethylnaphthalene in 100 ml of absolute acetonitrile was treated with 13.8 g (100 mmol) of anhydrous potassium carbonate and thereafter the mixture was stirred at room temperature for 18 hours. The reaction mixture was filtered, the filtrate was poured on to ice-water and adjusted to pH 2-3 with concentrated hydrochloric acid; the aqueous phase was extracted three times with 200 ml of ethyl acetate each time, the organic phase was washed once with 200 ml of water, dried over magnesium sulfate, filtered and concentrated in a water-jet vacuum. The crude product (5.5 g) was chromatographed on silica gel with hexane and ethyl acetate as the eluent. The product was recrystallized from ethyl acetate and hexane. There were obtained 2.27 g (61% of theory) of tert-butyl (3RS)-3-(naphthalen-2-ylmethylthio)-4-oxo-piperidine-1-carboxylate as a colorless solid; MS: 371 (M)⁺.

c) 0.31 g (12.8 mg atoms) of magnesium shavings were suspended in 5 ml of absolute tetrahydrofuran under argon, then reacted at reflux with a solution of 1.75 g (10 mmol) of 4-bromo-fluorobenzene in 10 ml of tetrahydrofuran. After the reaction had died down a solution of 1.86 g (5 mmol) of tert-butyl (3RS)-3-(naphthalen-2-ylmethylthio)-4-oxo-piperidine-1-carboxylate in 10 ml of tetrahydrofuran was added dropwise at room temperature and the mixture was then stirred for a further 4 hours. After hydrolysis with 10 ml of water the reaction mixture was worked-up by extraction with ethyl acetate. The organic phase was dried over magnesium sulfate, filtered and the solvent was distilled off in a water-jet vacuum. The crude product (2.6 g) was chromatographed on silica gel with hexane and ethyl acetate as the eluent. There were obtained 1.45 g (62% of theory) of tert-butyl (3RS,4SR or 3RS,4RS)-4-(4-fluorophenyl)-4-hydroxy-3-(naphthalen-2-ylmethylthio)-piperidine-1-carboxylate as a colorless solid, MS: 468 (M+H)⁺, and 0.37 g (16% of theory) of tert-butyl (3RS,4RS or 3RS,4SR)-4-(4-fluorophenyl)-4-hydroxy-3-(naphthalen-2-ylmethylthio)-piperidine-1-carboxylate as a colorless solid; MS: 468 (M+H)⁺.

d) A solution of 0.23 g (0.5 mmol) of tert-butyl (3RS,4SR or 3RS,4RS)-4-(4-fluorophenyl)-4-hydroxy-3-(naphthalen-2-ylmethylthio)-piperidine-1-carboxylate in 5 ml of absolute methanol was treated with 1 ml of hydrochloric acid in methanol (1.4 molar) and thereafter stirred at 50° C. for 3 hours. After distillation of the solvent in a water-jet vacuum the product was recrystallized from methanol. There was thus obtained 0.18 g (89% of theory) of (3RS,4SR or 3RS,4RS)-4-(4-fluorophenyl)-4-hydroxy-3-(naphthalen-2-ylmethylthio)-piperidine hydrochloride as a pale yellow solid; MS: 368 (M+H)⁺.

### Example 70

A solution of 0.23 g (0.5 mmol) of tert-butyl (3RS,4RS or 3RS,4SR)-4-(4-fluorophenyl)-4-hydroxy-3-(naphthalen-2-yl-methylthio)-piperidine-1-carboxylate [Example 70(c)] in 5 ml of absolute methanol was treated with 1 ml of hydrochloric acid in methanol (1.4 molar) and thereafter stirred at 50° C. for 3 hours. After distillation of the solvent in a water-jet vacuum the residue was partitioned between methylene chloride and water, neutralized with saturated sodium bicarbonate solution and extracted; the organic phase was dried over magnesium sulfate, filtered and concentrated. The crude product (0.15 g) was chromatographed on silica gel with methylene chloride and methanol as the eluent. There was thus obtained 0.041 g (22% of theory) of (3RS,4RS or 3RS,4SR)-4-(4-fluorophenyl)-4-methoxy-3-(naphthalen-2-ylmethylthio)-piperidine as a yellow oil, MS: 381 (M)⁺, and 0.067 g (37% of theory) of (3RS,4RS or 3RS,4SR)-4-(4-fluorophenyl)-4-hydroxy-3-(naphthalen-2-ylmethylthio)-piperidine as a pale yellow solid; MS: 367 (M)⁺.

### Example 71

(a) 2.33 g (10.0 mmol) of benzyl rac-3-aza-7-oxa-bicyclo-[4.1.0]heptane-3-carboxylate [S. V. D'Andrea et al., J. Org. Chem. (1991), 56(9), 3133-3137] and 1.88 g (20.0 mmol, 2 eq.) of phenol were dissolved in 30 ml of acetonitrile and treated at room temperature with 10.0 ml of 2 N sodium hydroxide solution. This solution was stirred at 95° C. for 5 hours. Subsequently, the solution, cooled to room temperature, was treated with 60 ml of water and extracted three times with methylene chloride. The organic phases were washed with 100 ml of 2 N sodium hydroxide solution and twice with water, dried over magnesium sulfate, evaporated under reduced pressure and dried in a high vacuum. The crude product (3.05 g) was separated on silica gel using a 7:3 mixture of hexane and ethyl acetate as the eluent. This yielded 2.06 g (63% of theory) of benzyl (3RS,4RS)-3-hydroxy-4-phenoxy-piperidine-1-carboxylate as a colorless oil; MS: 327 (M)⁺.

(b) A dispersion of 262 mg (6.0 mmol, 2 eq.) of sodium hydride (60% dispersion in refined oil) in 40 ml of dimethyl sulfoxide was treated with a solution of 982 mg (3.0 mmol, 1 eq.) of benzyl (3RS,4RS)-3-hydroxy-4-phenoxy-piperidine-1-carboxylate in 67 ml of dimethyl sulfoxide. This mixture was stirred at 40° C. for 2 hours, then cooled to room temperature and treated dropwise with a solution of 1326 mg (6.0 mmol, 2 eq.) of 2-bromo-methylnaphthalene in 40 ml of dimethyl sulfoxide and stirred at room temperature for 4 hours. The mixture was poured into 1 l of an ice/water mixture, stirred for 10 minutes and extracted three times with diethyl ether. The organic phases were washed once with water and once with saturated sodium chloride solution, dried over magnesium sulfate, evaporated under reduced pressure and dried in a high vacuum. The pale yellow oil (1.75 g) was separated on silica gel using a 4:1 mixture of hexane and ethyl acetate as the eluent. This yielded 647 mg (46% of theory) of benzyl (3RS,4RS)-3-(naphthalen-2-ylmethoxy)-4-phenoxy-piperidine-1-carboxylate as an amorphous solid; MS: 476 (M-benzyl)⁺.

(c) 30 mg, 0.065 mmol) of benzyl (3RS,4RS)-3-(naphthalen-2-ylmethoxy)-4-phenoxy-piperidine-1-carboxylate were dissolved in 1.6 ml of tetrahydrofuran, cooled to 0° C. and treated in succession within 25 minutes with a solution of 95 ml (0.32 mmol, approximately 5 eq.) of a 70% sodium dihydrido-bis-(2-methoxyethoxy)-aluminate solution (SDMA) in toluene and 1.6 ml of tetrahydrofuran. This reaction solution was stirred at 0° C. for 2.5 hours. Subsequently, it was poured into a mixture of saturated potassium sodium tartrate solution and ice and extracted four times with 50 ml of methylene chloride each time. The organic phases were washed twice with water, evaporated under reduced pressure and dried in a high vacuum. The orange colored resin was separated on silica gel using a 95:5 mixture of methylene chloride and methanol as the eluent. There were obtained 10 mg (47% of theory) of (3RS,4RS)-3-(naphthalen-2-ylmethoxy)-4-phenoxy-piperidine as a colorless, amorphous solid; R_{f}: 0.38 (SiO₂, methylene chloride:methanol=9:1).

### Example 72

The following compounds were obtained in an analogous manner to that described in Example 71 by cleavage of the benzyloxycarbonyl group:

1)—(3RS,4RS)-4-(4-Bromophenoxy)-3-(naphthalen-2-yl-methoxy)-piperidine as a colorless, amorphous solid, MS: 412.4, 414 (M+H)⁺, from benzyl (3RS,4RS)-4-(4-bromo-phenyl)-3-(naphthalen-2-ylmethoxy)-piperidine-1-carboxylate;

2)—(3RS,4RS)-4-(4-chlorophenylsulfanyl)-3-(naphthalen-2-ylmethoxy)-piperidine as a colorless, amorphous solid, MS: 384 (M+H)⁺, from benzyl (3RS,4RS)-4-(4-chloro-phenylsulfanyl)-3-(naphthalen-2-ylmethoxy)-piperidine-1-carboxylate.

The derivatives used as the starting materials were obtained as follows:

(a) In an analogous manner to that described in Example 71 (a)-(b), from benzyl rac.-3-aza-7-oxa-bicyclo[4.1.0]heptane-3-carboxylate by reaction with 4-bromophenol there was obtained (3RS,4RS)-1-[4-(4-bromo-phenoxy)-3-hydroxy-piperidin-1-yl]-2-phenyl-ethanone as a colorless solid [R_{f}: 0.40 (SiO₂, methylene chloride:ethyl acetate=2:1)], alkylation of which with 2-bromomethylnaphthalene gave benzyl (3RS,4RS)-4-(4-bromo-phenyl)-3-(naphthalen-2-ylmethoxy)-piperidine-1-carboxylate as a colorless solid; MS: 454, 456 (M-benzyl)⁺.

(b) A mixture of 2.33 g (10.0 mmol) of benzyl rac.-3-aza-7-oxa-bicyclo[4.1.0]heptane-3-carboxylate, 2.89 g (20.0 mmol, 2 eq.) of p-chlorothiophenol and 10.0 ml of 2 N sodium hydroxide solution in 20.5 ml of acetonitrile was boiled under reflux for 4 hours. Subsequently, the solution, cooled to room temperature, was treated with 25 ml of water and extracted three times with methylene chloride. The organic phases were washed once with 1 N sodium hydroxide solution and twice with water, dried over magnesium sulfate, evaporated under reduced pressure and dried in a high vacuum. The crude product (3.55 g) was separated on silica gel using a 9:1 mixture of methylene chloride and ethyl acetate as the eluent. This yielded 1.89 g (46% of theory) of benzyl (3RS,4RS)-4-(4-chloro-phenylsulfanyl)-3-hydroxy-piperidine-1-carboxylate as a colorless solid, MS: 377 (M)⁺, and 169 mg (4%) of benzyl (3RS,4SR)-4-(4-chloro-phenylsulfanyl)-3-hydroxy-piperidine-1-carboxylate.

(c) In an analogous manner to that described in Example 71 (b), from benzyl (3RS,4RS)-4-(4-chloro-phenylsulfanyl)-3-hydroxy-piperidine-1-carboxylate by alkylation with 2-bromomethyl-naphthalene there was obtained benzyl (3RS,4RS)-4-(4-chloro-phenylsulfanyl)-3-(naphthalen-2-ylmethoxy)-piperidine-1-carboxylate as a colorless solid; MS: 518 (M+H)⁺.

### Example 73

a) A solution of 5.0 g (16.9 mmol) of tert-butyl (3RS,4RS)-4-(4-fluoro-phenyl)-3-hydroxy-piperidine-1-carboxylate in 50 ml of methylene chloride was treated with 240 mg (1.96 mmol) of 4-dimethylamino-pyridine and 4.2 ml (29 mmol) of triethylamine and cooled to 0° C. Subsequently, 4.65 g (24.4 mmol) of 2-naphthoyl chloride were added portionwise and the reaction mixture was stirred at room temperature for 18 hours. Thereupon, the reaction mixture was treated with ice-water and extracted with methylene chloride. The combined methylene chloride phases were dried over magnesium sulfate, filtered and the solvent was distilled off in a water-jet vacuum. The crude product was chromatographed on silica gel with methylene chloride as the eluent. The thus-obtained product fractions were recrystallized from ether and n-hexane. There were obtained 7.4 g (97% of theory) of tert-butyl (3RS,4RS)-4-(4-fluoro-phenyl)-3-(naphthalen-2-ylcarbonyloxy)-piperidine-1-carboxylate as a colorless solid; MS: 450 (M+H)⁺.

b) A solution of 2.7 ml (24.6 mmol) of titanium tetrachloride in 18 ml of methylene chloride was added dropwise at 0° C. under argon and with the exclusion of moisture to 30 ml of tetrahydrofuran, a yellow suspension resulting. After warming to room temperature 15 ml (95 mmol) of tetramethylethylenediamine were added and the reaction mixture was stirred for 10 minutes. After the addition of 3.6 g (55 mmol) of zinc dust the mixture was stirred at room temperature for a further 30 minutes. Thereupon, a solution of 2.1 ml (30 mmol) of dibromomethane and 2.7 g (6.0 mmol) of tert-butyl (3RS,4RS)-4-(4-fluoro-phenyl)-3-(naphthalen-2-ylcarbonyloxy)-piperidine-1-carboxylate dissolved in 30 ml of tetrahydrofuran was added dropwise in such a manner that the temperature did not rise above 36° C. Subsequently, the reaction mixture was stirred at room temperature for 60 hours, then poured into saturated ammonium chloride solution and extracted with ether. The combined ether phases were dried over magnesium sulfate, concentrated and the residue was chromatographed on silica gel using a 99:1 mixture of methylene chloride and triethylamine as the eluent. Therefrom there were obtained 1.23 g (46% of theory) of tert-butyl (3RS,4RS)-4-(4-fluoro-phenyl)-3-(1-naphthalen-2-yl-vinyloxy)-piperidine-1-carboxylate as an amorphous colorless solid; MS: 448 (M+H)⁺.

c) A solution of 70 mg (0.156 mmol) of tert-butyl (3RS,4RS)-4-(4-fluoro-phenyl)-3-(1-naphthalen-2-yl-vinyloxy)-piperidine-1-carboxylate in 10 ml of tetrahydrofuran was treated with 0.2 ml of triethylamine and 100 mg of palladium on charcoal and hydrogenated in a hydrogen atmosphere at room temperature and normal pressure. Subsequently, the reaction mixture was suction filtered over a 0.8.mu. cellulose filter and the solvent was evaporated in a water-jet vacuum. There were obtained 68.4 mg (97% of theory) of a mixture of tert-butyl (3RS,4RS)-4-(4-fluoro-phenyl)-3-[(RS)- and -[(SR)-1-naphthalen-2-yl-ethoxy)-piperidine-1-carboxylate as an amorphous colorless solid; MS: 450 (M+H)⁺.

d) 69 mg (0.153 mmol) of a mixture of tert-butyl (3RS,4RS)-4-(4-fluoro-phenyl)-3-[(RS)- and -[(SR)-1-naphthalen-2-yl-ethoxy]-piperidine-1-carboxylate were dissolved in 2 ml of methylene chloride, treated with 104 mg (0.46 mmol) of anhydrous zinc bromide and stirred at room temperature for 2.5 hours. Thereupon, the reaction mixture was poured into aqueous sodium carbonate solution and this was extracted with methylene chloride. The combined methylene chloride phases were dried over magnesium sulfate, concentrated and the thus-obtained residue was chromatographed on silica gel with a 9:1 mixture of methylene chloride and methanol as the eluent. There were thus obtained 22.1 mg (41% of theory) of (3RS,4RS)-4-(4-fluoro-phenyl)-3-[(RS)- or -[(SR)-1-naphthalen-2-yl-ethoxy]-piperidine as an amorphous yellowish solid; MS: 350 (M+H)⁺, and 13.6 mg (25% of theory) of (3RS,4RS)-4-(4-fluoro-phenyl)-3-[(SR)- or -[(RS)-1-naphthalen-2-yl-ethoxy]-piperidine as an amorphous colorless solid; MS: 350 (M+H)⁺.

### Example 74

a) 13.8 g (100 mmol) of potassium carbonate were added to a solution of 10.0 g (46.8 mmol) of 4-(4-fluoro-phenyl)-1,2,3,6-tetrahydro-pyridine hydrochloride in 400 ml of ethanol and the reaction mixture was subsequently heated to reflux temperature. A solution of 5.8 ml (49 mmol) of benzyl bromide in 100 ml of ethanol was added dropwise within one hour and thereafter the mixture was stirred at this temperature for a further 1 hour. The reaction mixture was cooled to room temperature and filtered, the filtrate was extracted with water and ethyl acetate and finally the organic phase was dried over magnesium sulfate. After evaporation in a water-jet vacuum the crude product obtained was chromatographed on silica gel with hexane and ethyl acetate as the eluent. There were obtained 8.90 g (71% of theory) of 1-benzyl-4-(4-fluoro-phenyl)-1,2,3,6-tetrahydro-pyridine; MS: 267 (M)⁺.

b) 4.5 g (16.8 mmol) of 1-benzyl-4-(4-fluoro-phenyl)-1,2,3,6-tetrahydro-pyridine were suspended in 55 ml of water, then partially dissolved by the addition of 70 ml of concentrated hydrochloric acid, 1.36 g (45.3 mmol) of paraformaldehyde were added and the mixture was stirred at 100° C. for 5 hours. After cooling to room temperature the mixture was adjusted to pH 5-6 with sodium hydroxide solution and the product was extracted twice with 100 ml of ethyl acetate. The organic phases were washed once with 100 ml of water, dried over magnesium sulfate, filtered and concentrated in a water-jet vacuum. The thus-obtained crude product was chromatographed on silica gel with hexane and ethyl acetate as the eluent (sic). There were obtained 3.91 g (78% of theory) of (RS)-[1-benzyl-4-(4-fluoro-phenyl)-1,2,3,6-tetrahydro-pyridin-3-yl]-methanol; MS: 297 (M)⁺.

c) 58 ml (203 mmol) of sodium dihydrido-bis-(2-methoxy-ethoxy)aluminate (70% in toluene) were added dropwise under argon at room temperature while stirring to a solution of 17.4 g (58.5 mmol) of (RS)-[1-benzyl-4-(4-fluoro-phenyl)-1,2,3,6-tetrahydro-pyridin-3-yl]-methanol in 580 ml of absolute toluene. Subsequently, the mixture was stirred at 80° C. for 4 hours. 100 ml of water were added dropwise to the reaction mixture at room temperature, with working-up thereafter being carried out by extraction with water and ethyl acetate. The crude product was chromatographed on silica gel with hexane and ethyl acetate as the eluent. There were obtained 3.90 g (44% of theory) of (3RS,4SR)-[1-benzyl-4-(4-fluoro-phenyl)-piperidin-3-yl]-methanol; MS: 300 (M+H)⁺.

d) 6.86 g (22.9 mmol) of (3RS,4SR)-[1-benzyl-4-(4-fluoro-phenyl)-piperidin-3-yl]-methanol were hydrogenated at room temperature in 70 ml of methanol with the addition of 1.5 g of Pd-charcoal (10%). After filtration of the catalyst the solvent was distilled off in a water-jet vacuum. There were thus obtained 4.79 g (100% of theory) of (3RS,4SR)-[4-(4-fluoro-phenyl)-piperidin-3-yl]-methanol; MS: 210 (M+H)⁺.

e) 4.20 g (50 mmol) of sodium hydrogen carbonate and 20 ml of water were added to a solution of 4.89 g (23.4 mmol) of (3RS,4SR)-[4-(4-fluoro-phenyl)-piperidin-3-yl]-methanol in 60 ml of dioxan, then 6.10 g (28 mmol) of di-tert-butyl dicarbonate were introduced portionwise and the mixture was stirred at room temperature for 4 hours. The reaction mixture was poured on to ice-water and the product was extracted twice with 200 ml of ethyl acetate each time; the organic phases were washed once with 300 ml of water, dried over magnesium sulfate, filtered and concentrated in a water-jet vacuum. The thus-obtained crude product was chromatographed on silica gel with methylene chloride and methanol as the eluent. There were obtained 7.03 g (97% of theory) of tert-butyl (3RS,4SR)-4-(4-fluoro-phenyl)-3-hydroxymethyl-piperidine-1-carboxylate; MS: 309 (M)⁺.

f) 3.28 ml (46.2 mmol) of dimethyl sulfoxide were added dropwise at −70° C. under argon to a solution of 2.34 ml (27.3 mmol) of oxalyl chloride in 250 ml of methylene chloride. After 30 minutes 6.50 g (21 mmol) of tert-butyl (3RS,4SR)-4-(4-fluoro-phenyl)-3-hydroxymethyl-piperidine-1-carboxylate dissolved in 75 ml of methylene chloride were added dropwise and the mixture was stirred at −70° C. for 2 hours. Subsequently, 7.25 ml (52.5 mmol) of triethylamine were added dropwise. The reaction mixture was warmed to room temperature during 3 hours and subsequently extracted with water and methylene chloride. After drying over magnesium sulfate and evaporation in a water jet vacuum the product was purified by recrystallization from n-hexane. There were thus obtained 5.51 g (85% of theory) of tert-butyl (3RS,4SR)-4-(4-fluoro-phenyl)-3-formyl-piperidine-1-carboxylate; MS: 279 (M-CO)⁺.

g) 28.0 ml (54 mmol) of hexabutyldistannate were placed in 150 ml of tetrahydrofuran at 0° C. under argon. Thereto there were added dropwise 31.3 ml (50 mmol) of n-butyllithium solution (1.6 M in n-hexane). After 30 minutes a solution of 11.1 g (50 mmol) of 2-bromomethyl-naphthalene in 50 ml of tetrahydrofuran were added dropwise and thereafter the mixture was stirred at room temperature. After 2 hours the solvent was distilled off in a water-jet vacuum and the residue was chromatographed on silica gel using hexane and ethyl acetate as the eluent. There were obtained 16.8 g (78% of theory) of tributyl-naphthalen-2-ylmethyl-stannate; MS: 432 (M+H)⁺.

h) 16.8 g (38.9 mmol) of tributyl-naphthalen-2-ylmethyl-stannate were dissolved in 150 ml of tetrahydrofuran under argon. Thereafter, 12.5 ml (20 mmol) of n-butyllithium solution (1.6 M in n-hexane) were added dropwise at −78° C. After 30 minutes a solution of 4.80 g (15.6 mmol) of tert-butyl (3RS,4SR)-4-(4-fluoro-phenyl)-3-formyl-piperidine-1-carboxylate in 70 ml of tetrahydrofuran was added dropwise at −78° C. and the reaction mixture was stirred for a further 2 hours. Subsequently, the mixture was warmed to room temperature for 3 hours and stirred for a further 18 hours. After distillation of the solvent in a water-jet vacuum the reaction mixture was partitioned between water and methylene chloride, the organic phase was dried over magnesium sulfate and concentrated. The crude product was chromatographed on silica gel with hexane and ethyl acetate as the eluent. There were obtained 5.50 g (78% of theory) of a mixture of tert-butyl (3RS,4SR)-4-(4-fluoro-phenyl-3-[(RS)- and -[(SR)-1-hydroxy-2-naphthalen-2-yl-ethyl]-piperidine-1-carboxylate; MS: 450 (M+H)⁺.

i) 0.45 g (1 mmol) of the mixture of tert-butyl (3RS,4SR)-4-(4-fluoro-phenyl-3-[(RS)- and -[(SR)-1-hydroxy-2-naphthalen-2-yl-ethyl]-piperidine-1-carboxylate was dissolved in 10 ml of methanol. 2.0 ml (2.8 mmol) of HCl in methanol were added to the solution and the reaction mixture was stirred at 50° C. for 1 hour. After distillation of the solvent in a water-jet vacuum the residue was recrystallized from methanol and ether. There was obtained 0.16 g (42% of theory) of a mixture of (RS)- and (SR)-1-[(3RS,4SR)-4-(4-fluoro-phenyl)-piperidin-3-yl]-2-naphthalen-2-yl-ethanol hydrochloride (1:1) in the form of colorless crystals; MS: 350 (M+H)⁺.

### Example 75

a) 0.45 g (1 mmol) of a mixture of tert-butyl (3RS,4SR)-4-(4-fluoro-phenyl-3-[(RS)- and -[(SR)-1-hydroxy-2-naphthalen-2-yl-ethyl]-piperidine-1-carboxylate was dissolved in 5 ml of dimethylformamide under argon, then treated in succession with 0.16 g (1.2 mmol) of 4-dimethylamino-pyridine and 0.14 ml (1.2 mmol) of benzoyl chloride and stirred at room temperature for 16 hours. The reaction mixture was subsequently worked-up by extraction with ice-water and methylene chloride. The thus-obtained crude product was chromatographed on silica gel with hexane and ethyl acetate as the eluent. There were obtained 0.27 g (49% of theory) of tert-butyl (3RS,4SR)-3-[(RS)- or -[(SR)-1-benzoyloxy-2-naphthalen-2-yl-ethyl]-4-(4-fluoro-phenyl)-piperidine-1-carboxylate, MS: 554 (M+H)⁺, and 0.19 g (34% of theory) of tert-butyl (3RS,4SR)-3-[(SR)- or -[(RS)-1-benzoyloxy-2-naphthalen-2-yl-ethyl]-4-(4-fluoro-phenyl)-piperidine-1-carboxylate, MS: 554 (M+H)⁺.

b) 0.15 g (0.27 mmol) of tert-butyl (3RS,4SR)-3-[(SR)- or -[(RS)-1-benzoyloxy-2-naphthalen-2-yl-ethyl]4-(4-fluoro-phenyl)-piperidine-1-carboxylate was stirred at 50° C. for 1 hour in 10 ml of methanol with the addition of 1.0 ml (1.4 mmol) of HCl in methanol. After distillation of the solvent in a water-jet vacuum and subsequent drying in a high vacuum there was obtained 0.12 g (91% of theory) of (SR)- or (RS)-1-[(3RS,4SR)-4-(4-fluoro-phenyl)-piperidin-3-yl]-2-naphthalen-2-yl-ethyl benzoate hydrochloride (1:1) as a colorless foam; MS: 454 (M+H)⁺.

### Example 76

a) 0.25 g (0.45 mmol) of tert-butyl (3RS,4SR)-3-[(RS)- or -[(SR)-1-benzoyloxy-2-naphthalen-2-yl-ethyl]-4-(4-fluoro-phenyl)-piperidine-1-carboxylate was reacted with 1.2 ml (1.68 mmol) of HCl in methanol in analogy to Example 75(b). There was thus obtained 0.21 g (95% of theory) of (RS)- or (SR)-1-[(3RS,4SR)-4-(4-fluoro-phenyl)-piperidin-3-yl]-2-naphthalen-2-yl-ethyl benzoate hydrochloride (1:1) as a colorless foam; MS: 454 (M+H)⁺.

### Example 77

a) 0.45 g (1 mmol) of a mixture of tert-butyl (3RS,4SR)-4-(4-fluoro-phenyl-3-[(RS)- and -[(SR)-1-hydroxy-2-naphthalen-2-yl-ethyl)-piperidin-1-carboxylate was reacted with 0.09 ml (1.26 mmol) of acetyl chloride and 0.16 g (1.3 mmol) of 4-dimethylamino-pyridine in analogy to Example 75(a). There was thus obtained 0.38 g (77% of theory) of a mixture of tert-butyl (3RS,4SR)-3 [(RS)- and -[(SR)-1-acetyloxy-2-naphthalen-2-yl-ethyl]-4-(4-fluoro-phenyl)-piperidine-1-carboxylate, which was used directly in the next step.

b) 0.15 g (0.31 mmol) of the mixture of tert-butyl (3RS,4SR)-3[(RS)- and -[(SR)-1- of acetyloxy-2-naphthalen-2-yl-ethyl]-4-(4-fluoro-phenyl)-piperidine-1-carboxylate was treated in 10 ml of methylene chloride which 0.5 ml (6.5 mmol) of trifluoroacetic acid and the mixture was stirred at room temperature for 16 hours. After distillation of the solvent in a water-jet vacuum and subsequent drying in a high vacuum there was obtained 0.15 g (96% of theory) of a mixture of (RS)- and (SR)-1-[(3RS,4SR)-4-(4-fluoro-phenyl)-piperidin-3-yl]-2-naphthalen-2-yl-ethyl acetate trifluoroacetate (1:1) as a colorless foam; MS: 392 (M+H)⁺.

### Example 78

a) 0.45 g (1 mmol) of a mixture of tert-butyl (3RS,4SR)-4-(4-fluoro-phenyl-3-[(RS)- and -[(SR)-1-hydroxy-2-naphthalen-2-yl-ethyl]-piperidine-1-carboxylate was dissolved in 10 ml of dimethylformamide under argon and 0.07 g (1.6 mmol) of sodium hydride dispersion (55% in mineral oil) was added thereto at room temperature while stirring. After 1 hour 0.14 ml (1.2 mmol) of benzyl bromide was added dropwise and the reaction mixture was stirred at room temperature for 18 hours. Subsequently, the mixture was worked-up by extraction with ice-water and methylene chloride. The thus-obtained crude product was chromatographed on silica gel with hexane and ethyl acetate as the eluent. There were obtained 0.21 g (39% of theory) of tert-butyl (3RS,4SR)-3-[(RS)- or -[(SR)-1-benzyloxy-2-naphthalen-2-yl-ethyl]-4-(4-fluoro-phenyl)-piperidine-1-carboxylate, MS: 540 (M+H)⁺, and 0.17 g (31% of theory) of tert-butyl (3RS,4SR)-3-[(SR)- or -[(RS)-1-benzyloxy-2-naphthalen-2-yl-ethyl]-4-(4-fluoro-phenyl)-piperidine-1-carboxylate, MS: 540 (M+H)⁺.

b) 0.12 g (0.22 mmol) of tert-butyl 3RS,4SR)-3-[(RS)- or -[(SR)-1-benzyloxy-2-naphthalen-2-yl-ethyl]-4-(4-fluoro-phenyl)-piperidine-1-carboxylate was reacted with 1.0 ml (1.40 mmol) of HCl in methanol in analogy to Example 75(b). There was thus obtained 0.10 g (95% or theory) of (3RS,4SR)-3-[(RS)- or -[(SR)-1-benzyloxy-2-naphthalen-2-yl-ethyl]-4-(4-fluoro-phenyl)-piperidine hydrochloride (1:1) as a colorless foam; MS: 440 (M+H)⁺.

### Example 79

a) 0.16 g (0.3 mmol) of tert-butyl (3RS,4SR)-3-[(SR)- or [(RS)-1-benzyloxy-2-naphthalen-2-yl-ethyl]-4-(4-fluoro-phenyl)-piperidine-1-carboxylate was reacted with 1.0 ml (1.40 mmol) of HCl in methanol in analogy to Example 75(b). After distillation of the solvent in a water-jet vacuum the residue was recrystallized from methanol and diethyl ether. There was thus obtained 0.085 g (60% of theory) of (3RS,4SR)-3-[(SR)- or -[(RS)-1-benzyloxy-2-naphthalen-2-yl-ethyl]-4-(4-fluoro-phenyl)-piperidine hydrochloride (1:1) as a white solid; MS: 440 (M+H)⁺.

### Example 80

a) 0.45 g (1 mmol) of a mixture of tert-butyl (3RS,4SR)-4-(4-fluoro-phenyl-3-[(RS)- and -[(SR)-1-hydroxy-2-naphthalen-2-yl-ethyl]-piperidine-1-carboxylate was dissolved in 10 ml of dimethylformamide under argon, then 0.07 g (1.6 mmol) of sodium hydride dispersion (55% in mineral oil) was added at room temperature while stirring. After 1 hour 0.27 g (1.2 mmol) of 2-(bromomethyl)-naphthalene was added and the mixture was stirred at room temperature for 72 hours. Subsequently, the mixture was extracted with ice-water and ethyl acetate, the organic phase was dried over magnesium sulfate and subsequently evaporated in a water-jet vacuum. The thus-obtained crude product was chromatographed on silica gel with hexane and ethyl acetate as the eluent. There was obtained 0.19 g (32% of theory) of tert-butyl (3RS,4SR)-4-(4-fluoro-phenyl)-3-[(SR)- or (RS)-2-naphthalen-2-yl-1-(naphthalen-2-ylmethoxy)-ethyl]-piperidine-1-carboxylate, which was used directly in the next step, and 0.09 g (15% of theory) of tert-butyl (3RS,4SR)-4-(4-fluoro-phenyl)-3-[(RS)- or (SR)-2-naphthalen-2-yl-1-(naphthalen-2-ylmethoxy)-ethyl]-piperidine-1-carboxylate, which was likewise used directly in the next step.

b) 0.19 g (0.32 mmol) of tert-butyl (3RS,4SR)-4-(4-fluoro-phenyl)-3-[(SR)- or (RS)-2-naphthalen-2-yl-1-(naphthalen-2-ylmethoxy)-ethyl]-piperidine-1-carboxylate was reacted with 1.0 ml (1.40 mmol) of HCl in methanol in analogy to Example 75(b). The reaction solution was poured on to ice-water, neutralized with saturated sodium hydrogen carbonate solution, the product was then extracted twice with 50 ml of methylene chloride. The organic phases were dried over magnesium sulfate, filtered and the solvent was distilled off in a water-jet vacuum. The thus-obtained crude product was chromatographed on silica gel with methylene chloride and methanol as the eluent. There was thus obtained 0.28 g (18% of theory) of (3RS,4SR)-4-(4-fluoro-phenyl)-3-[(SR)- or (RS)-2-naphthalen-2-yl-1-(naphthalen-2-ylmethoxy)-ethyl]-piperidine as an amorphous, colorless solid; MS: 490 (M+H)⁺.

### Example 81

a) 0.09 g (0.15 mmol) of tert-butyl (3RS,4SR)-4-(4-fluoro-phenyl)-3-[(RS)- or (SR)-2-naphthalen-2-yl-1-(naphthalen-2-ylmethoxy)-ethyl]-piperidine-1-carboxylate was reacted with 1.0 ml (1.40 mmol) of HCl in methanol in analogy to Example 75(b). The reaction solution was poured on to ice-water (sic), neutralized with saturated sodium hydrogen carbonate solution, then the product was extracted twice with 50 ml of methylene chloride. The organic phases were dried over magnesium sulfate, filtered and the solvent was distilled off in a water-jet vacuum. The thus-obtained crude product was chromatographed on silica gel with methylene chloride and methanol as the eluent. There was thus obtained 0.036 g (49% of theory) of (3RS,4SR)-4-(4-fluoro-phenyl)-3-[(RS)- or (SR)-2-naphthalen-2-yl-1-(naphthalen-2-ylmethoxy)-ethyl]-piperidine as an amorphous, colorless solid; MS: 490 (M+H)⁺.

### Example 82

a) A solution of 1.08 g (4.3 mmol) of (1RS,2RS,3RS,5SR)-3-(4-chloro-phenyl)-8-methyl-8-aza-bicyclo[3.2.1]octan-2-ol [J. Org. Chem. 35, 802, 1970)] in 5 ml of tetrahydrofuran was added dropwise to a suspension of 0.206 g (4.3 mmol) of sodium hydride (50% dispersion in refined oil) in 6 ml of tetrahydrofuran and the mixture was stirred at 50° C. for 60 minutes. Subsequently, the mixture was cooled to room temperature and treated with 0.95 g (4.3 mmol) of 2-bromomethylnaphthalene in 5 ml of tetrahydrofuran. After 2 hours at 50° C. the reaction solution was poured into 60 ml of ice-water and extracted three times with 25 ml of ethyl acetate. The organic phases were washed with saturated sodium chloride solution, dried over magnesium sulfate, filtered and evaporated. The residue was chromatographed on silica gel using a 95:5 mixture of methylene chloride and methanol as the eluent and yielded 1.04 g (62% of theory) of (1RS,2RS,3RS,5SR)-3-(4-chloro-phenyl)-8-methyl-2-(naphthalen-2-ylmethoxy)-8-aza-bicyclo[3.2.1]octane as a light yellow oil, R.sub.f: 0.43 (silica gel, methylene chloride;methanol=95:5), MS: 392 (M)⁺.

(b) A solution of 1.02 g (2.6 mmol) of (1RS,2RS,3RS,5SR)-3-(4-chloro-phenyl)-8-methyl-2-(naphthalen-2-ylmethoxy)-8-aza-bicyclo[3.2.1]octane in 40 ml of toluene was treated with 150 mg of potassium carbonate and heated to 100° C. Subsequently, 0.635 g (0.400 ml) (3 mmol) of 2,2,2-trichloroethyl chloroformate was added thereto and the mixture was stirred at 100° C. for 12 hours.

The reaction solution was evaporated under reduced pressure, the residue was taken up in 70 ml of ethyl acetate and washed with 30 ml of water and 30 ml of saturated sodium hydrogen carbonate solution. Drying over magnesium sulfate, filtration and evaporation yielded a colorless oil which was chromatographed on silica gel using a 3:2 mixture of hexane and ethyl acetate. There were obtained 1.14 g (79% of theory) of 2,2,2-trichloroethyl (1RS,2RS,3RS,5SR)-3-(4-chloro-phenyl)-2-(naphthalen-2-ylmethoxy)-8-aza-bicyclo[3.2.1]octane-8-carboxylate as a colorless oil, R_{f}: 0.38 (silica gel, hexane:ethyl acetate=3:2).

(c) A suspension of 1.14 g (2.06 mmol) of 2,2,2-trichloroethyl (1RS,2RS,3RS,5SR)-3-(4-chloro-phenyl)-2-(naphthalen-2-ylmethoxy)-8-aza-bicyclo[3.2.1]octane-8-carboxylate and 400 mg of zinc in 10 ml of acetic acid was stirred at room temperature for 12 hours. The reaction solution was diluted with 50 ml of water and extracted four times with 40 ml of methylene chloride. The organic phase was washed twice with 50 ml of 1 N sodium hydroxide solution, dried over sodium sulfate, filtered and evaporated. The residue was chromatographed on silica gel using a 9:1 mixture of methylene chloride and methanol as the eluent. There was obtained 0.480 g (61% of theory) of (1RS,2RS,3RS,5SR)-3-(4-chloro-phenyl)-2-(naphthalen-2-ylmethoxy)-8-aza-bicyclo[3.2.1]octane of m.p. 184-185°; MS: 379 (M+H)⁺.

### Example 83

The following compounds were prepared in an analogous manner to that described in Example 82(a)-(c):

1)—From (1RS,2RS,3RS,5SR)-3-(4-chloro-phenyl)-8-methyl-8-aza-bicyclo[3.2.1]octan-2-ol and 4-chloromethyl-biphenyl, (1RS,2RS,3RS,5SR)-2-(biphenyl-4-ylmethoxy)-3-(4-chloro-phenyl)-8-aza-bicyclo[3.2.1]octane, MS: 236 (M-C₁3H₁1)⁺, which was converted with hydrogen chloride in ethanol into the hydrochloride of m.p. 175-177′ (dec.);

2)—from (1RS,2RS,3RS,5SR)-3-(4-chloro-phenyl)-8-methyl-8-aza-bicyclo[3.2.1]octane-2-ol and 3,4-dichloro-1-chloromethylbenzene, 3-(4-chloro-phenyl)-2-(3,4-dichloro-benzyloxy)-8-aza-bicyclo[3.2.1]octane, MS: 236 (M-C₇H₅Cl₂)⁺, which was converted with hydrogen chloride in ethanol into the hydrochloride of m.p. 211-213° C.;

3)—from (1RS,2RS,3RS,5SR)-3-(4-chloro-phenyl)-8-methyl-8-aza-bicyclo[3.2.1]octane-2-ol and 1-chloro-methyl-4-methoxy-benzene, (1RS,2RS,3RS,5SR)-3-(4-chloro-phenyl)-2-(4-methoxy-benzyloxy)-8-aza-bicyclo[3.2.1]octane, MS: 358 (M+H)⁺, which was converted with methanesulfonic acid in dioxan/water and subsequent lyophilization into the corresponding methanesulfonate, R_{f}: 0.26 (silica gel, methylene chloride:methanol:ammonia=200:10:1);

4)—from (1RS,2RS,3RS,5SR)-3-(4-chloro-phenyl)-8-methyl-8-aza-bicyclo[3.2.1]octane-2-ol and 3-chloromethyl-benzo-[b]thiophene (J. Am. Chem. Soc. 71, 2856 (1949), 2-(benzo-[b]thiophen-2-ylmethoxy)-3-(4-chloro-phenyl)-8-aza-bicyclo-[3.2.1]octane, MS: 236 (M-C₈H₇S)⁺, which was converted with hydrogen chloride in ethanol into the hydrochloride of m.p. 196-198° C. (dec.);

5)—from (1RS,2RS,3RS,SR)-3-(4-chloro-phenyl)-8-methyl-8-aza-bicyclo[3.2.1]octane-2-ol and methyl 4′-bromomethyl-biphenyl-2-carboxylate [J. Med. Chem. 34, 2525 (1991), methyl (1RS,2RS,3RS,5SR)-4′-[3-(4-chloro-phenyl)-8-aza-bicyclo-(3.2.1]oct-2-yloxymethyl)-biphenyl-2-carboxylate, MS: 236 (M-C₁5H₁3O₂)⁺, which was converted with hydrogen chloride in ethanol into the hydrochloride of m.p. 101-103° C. (dec.);

6)—from (1RS,2RS,3RS,5SR)-3-(4-chloro-phenyl)-8-methyl-8-aza-bicyclo[3.2.1]octane-2-ol and 6-chloromethyl-1,1,4,4,-tetramethyl-1,2,3,4-tetrahydronaphthalene, (1RS,2RS,3RS,5SR)-3-(4-chloro-phenyl)-2-(5,5,8,8-tetramethyl-5,6,7,8-tetra hydro-naphthalen-2-ylmethoxy)-8-aza-bicyclo[3.2.1]octane, MS: 437 (M)⁺, which was converted with hydrogen chloride in ethanol into the hydrochloride of m.p. 87-90° C. (dec.).

### Example 84

(a) Cleavage of the N-methyl group by reacting 2,2,2-trichloroethyl chloroformate with (1RS,2RS,3RS,5SR)-3-(4-fluoro-phenyl)-8-methyl-8-aza-bicyclo[3.2.1]octane-2-ol, obtained analogously to the 4-chloro-phenyl derivative [J. Org. Chem. 35, 802, 1970)], was effected in an analogous manner to the procedure described in Example 12(c). There was thus obtained 2,2,2-trichloro-ethyl (1RS,2RS,3RS,5RS)-3-(4-fluoro-phenyl)-2-(2,2,2-trichloro-ethoxycarbonyloxy)-8-aza-bicyclo[3.2.1]octane-8-carboxylate as a yellowish solid; MS: 587, 589, 591, 593 (M+NH₄)⁺.

(b) By cleavage of the 2,2,2-trichloroethyl carbamate and 2,2,2-trichloroethyl carbonate from 2,2,2-trichloro-ethyl (1RS,2RS,3RS,5RS)-3-(4-fluoro-phenyl)-2-(2,2,2-trichloro-ethoxy-carbonyloxy)-8-aza-bicyclo[3.2.1]octane-8-carboxylate in an analogous manner to that described in Example 12(d) there was obtained (1RS,2RS,3RS,5RS)-3-(4-fluoro-phenyl)-8-aza-bicyclo[3.2.1]octan-2-ol as a colorless solid; MS: 221 (M)⁺.

(c) In analogy to the procedure described in Example 1(f), from (1RS,2RS,3RS,5RS)-3-(4-fluoro-phenyl)-8-aza-bicyclo[3.2.1]octane-2-ol by introduction of the BOC group there was obtained tert-butyl (1RS,2RS,3RS,5RS)-3-(4-fluoro-phenyl)-2-hydroxy-8-aza-bicyclo[3.2.1]octane-8-carboxylate as a colorless foam; MS: 265 (M-C₄H₈)⁺.

(d) Alkylation of tert-butyl (1RS,2RS,3RS,5RS)-3-(4-fluoro-phenyl)-2-hydroxy-8-aza-bicyclo[3.2.1]octane-8-carboxylate with 1-benzyloxy-3-chloromethyl-naphthalene (Example 19) analogously to the procedure described in Example 1(g) yielded tert-butyl (1RS,2RS,3RS,5SR)-2-(4-benzyloxy-naphthalen-2-ylmethoxy)-3-(4-fluoro-phenyl)-8-aza-bicyclo[3.2.1]octane-8-carboxylate as a colorless solid; MS: 568 (M+H)⁺.

(e) Cleavage of the BOC group using hydrogen chloride in ethanol analogously to the procedure described in Example 22(l) gave (1RS,2RS,3RS,5SR)-2-(4-benzyloxy-naphthalen-2-ylmethoxy)-3-(4-fluoro-phenyl)-8-aza-bicyclo[3.2.1]octane as a beige colored solid; MS: 468 (M+H)⁺.

### Example 85

A solution of 0.330 g (0.71 mmol) of methyl (1RS,2RS,3RS,5SR)-4′-[3-(4-chloro-phenyl)-8-aza-bicyclo[3.2.1]oct-2-yloxy-methyl]-biphenyl-2-carboxylate in 10 ml of ether was slowly added dropwise to a suspension of 33 mg of lithium aluminium hydride in 5 ml of ether and the mixture was stirred at room temperature for 4 hours. After the addition of aqueous ether and subsequently water the phases were separated, the organic phase was washed with saturated sodium chloride solution, dried over sodium sulfate, filtered and evaporated. The residue was chromatographed on silica gel using a 140:10:1 mixture of methylene chloride, methanol and ammonia as the eluent. There was obtained 0.210 g (68% of theory) of [4′-[3-(4-chloro-phenyl)-8-aza-bicyclo[3.2.1]oct-2-yloxymethyl]-biphenyl-2-yl]-ethanol as a colorless foam, R_{f}: 0.18 (methylene chloride:methanol:ammonia=140:10:1), MS: 434 (M+H)⁺.

### Example 86

The following compounds were prepared by cleavage of the BOC group:

1) From tert-butyl (3RS,4RS)-3-(naphthalen-2-ylmethoxy)-4-(4-prop-2-ynyloxy-phenyl)-piperidine-1-carboxylate with tri-fluoroacetic acid, (3RS,4RS)-3-(naphthalen-2-ylmethoxy)-4-[4-(prop-2-ynyloxy)-phenyl]-piperidine trifluoroacetate as a colorless solid; m.p.: 186° C. (dec.);

2) from tert-butyl (3RS,4RS)-4-(4-allyloxy-phenyl)-3-(naphthalen-2-ylmethoxy)-piperidine-1-carboxylate with hydrochloric acid in methanol, (3RS,4RS)-4-(4-allyloxy-phenyl)-3-naphthalen-2-ylmethoxy-piperidine as a light oil; MS: 374 (M+H)⁺;

3) from a mixture of tert-butyl (3RS,4RS)- and (3SR,4SR)-4-[4-[(RS)-2,2-dimethyl-1,3]dioxolan-4-ylmethoxy]-phenyl]-3-(naphthalen-2-ylmethoxy)-piperidine-1-carboxylate with hydrochloric acid in methanol with simultaneous cleavage of the isopropylidene group, a mixture of (RS)- and (SR)-3-[(3RS,4RS)-4-[3-(naphthalen-2-ylmethoxy)-piperidin-4-yl]-phenoxy]-propane-1,2-diol as a yellowish solid; MS: 408 (M+H)⁺;

4) from a mixture of tert-butyl (3RS,4RS)- and (3SR,4SR)-4-[4-[(RS)-2-hydroxy-3-phenoxy-propoxy]-phenyl]-3-(naphthalen-2-ylmethoxy)-piperidine-1-carboxylate with trifluoroacetic acid, a mixture of (RS)- and (SR)-1-[4-[(3RS,4RS)-3-(naphthalen-2-ylmethoxy)-piperidin-4-yl]-phenoxy]-3-phenoxy-propan-2-ol trifluoroacetate as a white solid; MS: 484 (M+H)⁺;

5) from a mixture of tert-butyl (3RS,4RS)- and (3SR,4SR)-4-[4-[(RS)-2-benzyloxy-3-methoxy-propoxy]-phenyl]-3-(naphthalen-2-ylmethoxy)-piperidine-1-carboxylate with trifluoroacetic acid, a mixture of (RS)- and (SR)-4-[(3RS,4RS)-4-(2-benzyloxy-3-methoxy-propoxy)-phenyl]-3-(naphthalen-2-ylmethoxy)-piperidine trifluoroacetate as a white solid; m.p.: 138-139° C.

6) from a mixture of tert-butyl (3RS,4RS)- and (3SR,4SR)-4-(4-[(RS)-2-hydroxy-3-phenylsulfanyl-propoxy]-phenyl]-3-(naphthalen-2-ylmethoxy)-piperidine-1-carboxylate with trifluoroacetic acid, a mixture of (RS)- and (SR)-1-[(3RS,4RS)-4-[3-(naphthalen-2-yloxymethyl)-piperidine-4-yl]-phenoxy]-3-phenylsulfanyl-propan-2-ol trifluoroacetate as a white solid; MS: 500 (M+H)⁺;

7) from a mixture of tert-butyl (3RS,4RS)- and (3SR,4SR)-4-[4-[(RS)-2-methoxy-3-phenoxy-propoxy)-phenyl]-3-(naphthalen-2-ylmethoxy)-piperidine-1-carboxylate with trifluoroacetic acid, a mixture of (3RS,4RS)-4-[4-[(RS) and [(SR)-2-methoxy-3-phenoxy-propoxy]-phenyl]-3-(naphthalen-2-ylmethoxy)-piperidine trifluoroacetate as a white solid; MS: 498 (M+H)⁺;

8) from a mixture of tert-butyl (3RS,4RS)- and (3SR,4SR)-4-[4-[(RS)-2-benzoyloxy-3-methoxy-propoxy]-phenyl]-3-(naphthalen-2-ylmethoxy)-piperidine-1-carboxylate with trifluoroacetic acid, a mixture of (RS)- and (SR)-1-methoxymethyl-2-[4-[(3RS,4RS)-3-(naphthalen-2-ylmethoxy)-piperidin-4-yl]-phenoxy]-ethyl benzoate trifluoroacetate as a white solid; MS: 526 (M+H)⁺;

9) from a mixture of tert-butyl (3RS,4RS)-4-[4-[(RS)- and [(SR)-(3-benzyloxy-2-methoxy-propoxy)-phenyl]-3-(naphthalen-2-ylmethoxy)-piperidine-1-carboxylate, a mixture of (3RS,4RS)-4-[4-[(RS)- and -[(SR)-3-benzyloxy-2-methoxypropoxy]-phenyl]-3-(naphthalen-2-ylmethoxy)-piperidine trifluoroacetate as a white solid; MS: 512 (M+H)⁺;

10) from tert-butyl (3RS,4RS)-3-(naphthalen-2-ylmethoxy)-4-{4-[2-(pyridin-3-ylmethoxy)-ethoxy]-phenyl}-piperidine-1-carboxylate with hydrogen chloride in methanol, (3RS,4RS)-3-(2-[4-[3-(naphthalen-2-ylmethoxy)-piperidine-4-yl]-phenoxy]-ethoxymethyl)-pyridine as a colorless resin; MS: 469 (M+H)⁺;

11) from tert-butyl (3RS,4RS)-4-{4-[2-(pyridin-3-ylmethoxy)-ethoxy]-phenyl}-3-[4-(2-trimethylsilanyl-ethoxymethoxy)-naphthalen-2-ylmethoxy]-piperidine-1-carboxylate with hydrogen chloride in methanol with simultaneous cleavage of the SEM group, (3RS,4RS)-3-(4-[4-[2-(pyridin-3-ylmethoxy)-ethoxy]-phenyl]-piperidin-3-yloxymethyl)-naphthalen-1-ol as a colorless resin; MS: 485(M+H)⁺;

12) from tert-butyl (3RS,4RS)-3-(naphthalen-2-ylmethoxy)-4-{4-[2-(pyridin-4-ylmethoxy)-ethoxy]-phenyl}-piperidine-1-carboxylate with hydrogen chloride in methanol, (3RS,4RS)-4-(2-{4-[3-(naphthalen-2-ylmethoxy)-piperidin-4-yl]-phenoxy}-ethoxymethyl)-pyridine as a colorless resin; MS: 469 (M+H)⁺;

13) from tert-butyl (3RS,4RS)-3-(naphthalen-2-ylmethoxy)-4-{4-[2-(pyridin-2-ylmethoxy)-ethoxy]-phenyl}-piperidine-1-carboxylate with hydrogen chloride in methanol, (3RS,4RS)-2-(2-{4-[3-(naphthalen-2-ylmethoxy)-piperidin-4-yl]-phenoxy}-ethoxymethyl)-pyridine as a colorless resin; MS: 469 (M+H)⁺;

14) from tert-butyl (3RS,4RS)-4-[4-(3-benzyloxy-propoxy)-phenyl]-3-(naphthalen-2-ylmethoxy)-piperidine-1-carboxylate with hydrogen chloride in methanol, (3RS,4RS)-4-[4-(3-benzyloxy-propoxy)-phenyl]-3-(naphthalen-2-ylmethoxy)-piperidine as a colorless resin; MS: 482 (M+H)⁺;

15) from tert-butyl (3RS,4RS)-3-(naphthalen-2-ylmethoxy)-4-[4-[3-(pyridin-2-ylmethoxy)-propoxy]-phenyl]-piperidine-1-carboxylate with hydrogen chloride in methanol, (3RS,4RS)-2-(3-{4-[3-(naphthalen-2-ylmethoxy)-piperidin-4-yl]-phenoxy}-propoxymethyl)-pyridine as a colorless solid; MS: 483 (M+H)⁺;

16) from tert-butyl (3RS,4RS)-3-(naphthalen-2-ylmethoxy)-4-[4-[3-(pyridin-2-ylmethoxy)-propyl]-phenyl]-piperidine-1-carboxylate with hydrogen chloride in methanol, (3RS,4RS)-2-[3-[4-[3-(naphthalen-2-ylmethoxy)-piperidin-4-yl]-phenyl]-prop oxymethyl]-pyridine as a colorless amorphous solid; MS: 467 (M+H)⁺;

17) from tert-butyl (3RS,4RS)-4-[4-[3-(benzyl-methyl-amino)-propoxy]-phenyl]-3-(naphthalen-2-ylmethoxy)-piperidine-1-carboxylate with hydrogen chloride in methanol, (3RS,4RS)-benzyl-methyl-(3-{4-[3-(naphthalen-2-ylmethoxy)-piperidin-4-yl]-phenoxy}-propyl)-amine as a colorless solid; MS: 495 (M+H)⁺;

18) from tert-butyl (3RS,4RS)-4-[4-[3-(benzothiazol-2-ylsulfonyl)-propoxy]-phenyl]-3-(1-methoxy-naphthalen-2-ylmethoxy)-piperidine-1-carboxylate with hydrogen chloride in methanol, (3RS,4RS)-2-[3-[4-[3-(1-methoxy-naphthalen-2-ylmethoxy)-piperidin-4-yl]-phenoxy]-propylsulfonyl]-benzothiazole as a colorless foam; MS: 571 (M+H)⁺;

19) from tert-butyl (3RS,4RS)-3-(naphthalen-2-ylmethoxy)-4-[4-(3-phenylsulfanyl-propyl)-phenyl]-piperidine-1-carboxylate with hydrogen chloride in methanol, (3RS,4RS)-3-(naphthalen-2-ylmethoxy)-4-[4-(3-phenylsulfanyl-propyl)-phenyl]-piperidine as a white solid; MS: 468 (M+H)⁺;

20) from tert-butyl (3RS,4RS)-4-{4-[3-(benzothiazol-2-ylsulfanyl)-propyl]-phenyl}-3-(naphthalen-2-ylmethoxy)-piperidine-1-carboxylate with hydrogen chloride in methanol, (3RS,4RS)-2-(3-{4-[3-(naphthalen-2-ylmethoxy)-piperidin-4-yl]-phenyl}-propylsulfanyl)-benzothiazole as a white solid; MS: 525 (M+H)⁺;

21) from tert-butyl (3RS,4RS)-4-{4-[2-(pyrimidin-2-ylsulfanyl)-ethyl]-phenyl}-3-[4-(2-trimethylsilanyl-ethoxymethoxy)-naphthalen-2-ylmethoxy]-piperidine-1-carboxylate with hydrogen chloride in methanol with simultaneous cleavage of the SEM group, (3RS,4RS)-3-[4-[4-[2-(pyrimidin-2-ylsulfanyl)-ethyl]-phenyl]-piperidin-3-yloxymethyl]-naphthalen-1-ol as colorless foam; MS: 472 (M+H)⁺;

22) from tert-butyl (3RS,4RS)-4-{4-[2-(pyridin-2-ylsulfanyl)-ethyl]-phenyl}-3-[4-(2-trimethylsilanyl-ethoxymethoxy)-naphthalen-2-ylmethoxy]-piperidine-1-carboxylate with hydrogen chloride in methanol with simultaneous cleavage of the SEM group, (3RS,4RS)-3-(4-{4-[2-(pyridin-2-ylsulfanyl)-ethyl]-phenyl}-piperidin-3-yl oxymethyl)-naphthalen-1-ol; MS: 471 (M+H)⁺;

23) from tert-butyl (3RS,4RS)-4-[4-[2-(benzothiazol-2-ylsulfanyl)-ethyl]-phenyl]-3-[4-(2-trim ethylsilanyl-ethoxy-methoxy)-naphthalen-2-ylmethoxy]-piperidine-1-carboxylate with hydrogen chloride in methanol with simultaneous cleavage of the SEM group, (3RS,4RS)-3-[4-[4-[2-(benzothiazol-2-ylsulfanyl)-ethyl]-phenyl]-piperidin-3-yloxymethyl]-naphthalen-1-ol as a colorless solid MS: 527 (M+H)⁺;

24) from tert-butyl (3RS,4RS)-3-(naphthalen-2-ylmethoxy)-4-[4-(pyridin-3-ylmethoxymethyl)-phenyl]-piperidine-1-carboxylate with hydrogen chloride in methanol, (3RS,4RS)-3-[4-[3-(naphthalen-2-ylmethoxy)-piperidin-4-yl]-benzyloxymethyl]-pyridine as a colorless foam; MS: 438 (M)⁺;

25) from a mixture of tert-butyl (3RS,4RS)-4-(4-{2-[(RS)-2- and (SR)-2-(4-fluoro-phenyl)-3-methyl-butyryloxy]-ethoxy]-phenyl)-3-(naphthalen-2-ylmethoxy)-piperidine-1-carboxylate with zinc bromide in methylene chloride, a mixture of (3RS,4RS)-2-[4-(3-naphthalen-2-ylmethoxy-piperidin-4-yl)-phenoxy]-ethyl (RS)- and (SR)-2-(4-fluoro-phenyl)-3-methyl-butyrate hydrobromide as a white solid; MS: 556 (M+H)⁺;

26) from tert-butyl (3RS,4RS)-4-[4-[2-(11-methyl-1H-pyrrol-2-carbonyloxy)-ethoxy]-phenyl]-3-(naphthalen-2-ylmethoxy)-piperidine-1-carboxylate with zinc bromide in methylene chloride, (3RS,4RS)-[4-[3-(naphthalen-2-yl]methoxy)-piperidin-4-yl]-phenoxy]-ethyl 1-methyl-1H-pyrrole-2-carboxylate hydrobromide as a beige solid; MS: 485 (M+H)⁺;

27) from tert-butyl (3RS,4RS)-4-[4-(3-benzoyloxy-propyl)-phenyl]-3-(naphthalen-2-ylmethoxy)-piperidine-1-carboxylate with hydrogen chloride in methanol, (3RS),4RS)-3-{4-[3-(naphthalen-2-ylmethoxy)-piperidin-4-yl]-phenyl}-propyl benzoate as a yellow syrup; MS: 480 (M+H)⁺;

28) from tert-butyl (3RS,4RS)-4-{4-[3-(3-methoxy-benzoyloxy)-propyl]-phenyl}-3-(naphthalen-2-ylmethoxy)-piperidine-1-carboxylate with hydrogen chloride in methanol, (3RS,4RS)-3-{4-[3-(naphthalen-2-ylmethoxy)-piperidin-4-yl]-phenyl}-propyl 3-methoxy-benzoate as a yellow solid; MS: 510 (M+H)⁺;

29) from tert-butyl (3RS,4RS)-4-[4-(3-methoxy-benzoyloxymethyl)-phenyl]-3-[1-(2-trimethylsilanyl-ethoxymethoxy)-naphthalen-2-ylmethoxy]-piperidine-1-carboxylate with hydrogen chloride in methanol with simultaneous cleavage of the SEM group, (3RS,4RS)-4-[3-(1-hydroxy-naphthalen-2-ylmethoxy)-piperidin-4-yl]-benzyl 3-methoxy-benzoate as a colorless foam; MS: 498 (M+H)⁺;

30) from tert-butyl (3RS,4RS)-4-(4-ethoxycarbonylmethoxy-phenyl)-3-(naphthalen-2-ylmethoxy)-piperidine-1-carboxylate with trifluoroacetic acid, ethyl (3RS,4RS)-[4-[3-(naphthalen-2-ylmethoxy)-piperidin-4-yl]-phenoxy]-acetate trifluoroacetate as a white solid; MS: 420 (M+H)⁺;

31) from tert-butyl (3RS,4RS)-4-[4-(benzylcarbamoyl-methoxy)-phenyl]-3-(naphthalen-2-ylmethoxy)-piperidine-1-carboxylate with trifluoroacetic acid, (3RS,4RS)-N-benzyl-2-[4-[3-(naphthalen-2-ylmethoxy)-piperidin-4-yl]-phenoxy]-acetamide trifluoroacetate as a white solid; m.p.: 185° C.;

32) from tert-butyl (3RS,4RS)-3-(naphthalen-2-ylmethoxy)-4-[4-(pyridin-2-ylcarbamoyloxy)-phenyl]-piperidine-1-carboxylate with trifluoroacetic acid, (3RS,4RS)-4-[3-(naphthalen-2-ylmethoxy)-piperidin-4-yl]-3-phenylpyridin-2-yl-carbamate trifluoroacetate as a white solid; m.p.: 158° C.;

33) from tert-butyl (3RS,4RS)-4-(4-carboxymethoxy-phenyl)-3-(naphthalen-2-ylmethoxy)-piperidine-1-carboxylate [Example 86 (ee)] with trifluoroacetic acid, (3RS,4RS)-[4-[3-(naphthalen-2-ylmethoxy)-piperidin-4-yl]-phenoxy]-acetic acid trifluoroacetate as a white colorless solid; m.p.: 183-184° C.;

34) from tert-butyl (3RS,4RS)-3-(naphthalen-2-ylmethoxy)-4-[4-(2-oxo-3-phenoxypropoxy)-phenyl]-piperidine-1-carboxylate with trifluoroacetic acid, (3RS,4RS)-1-[4-[3-(naphthalen-2-ylmethoxy)-piperidin-4-yl]-phenoxy]-3-phenoxy-propan-2-one trifluoroacetate as a white solid; m.p.: 145-146° C.;

35) from tert-butyl (3RS,4RS)-4-[4-(2-ethoxycarbonyl-ethyl)-phenyl]-3-(naphthalen-2-ylmethoxy)-piperidine-1-carboxylate [Example 24 (s)] with zinc bromide in methylene chloride, ethyl (3RS,4RS)-3-{4-[3-(naphthalen-2-ylmethoxy)-piperidin-4-yl]-phenyl}-propionate as a yellow syrup; MS: 418 (M+H)⁺;

36) from a mixture of tert-butyl (3RS,4RS)-4-[4-[(RS)-2- and [(SR)-2-hydroxy-2-phenyl-ethyl]-phenyl]-3-(naphthalen-2-ylmethoxy)-piperidine-1-carboxylate with hydrogen chloride in methanol, a mixture of (RS)- and (SR)-2-[4-[(3RS,4RS)-3-naphthalen-2-ylmethoxy-piperidin-4-yl]-phenyl]-1-phenyl-ethanol hydrochloride; MS: 438 (M+H)⁺;

37) from tert-butyl (3RS,4RS)-4-(4-fluoro-phenyl)-3-(2-{[(pyridin-2-carbonyl)-amino]-methyl}-benzyloxy)-piperidine-1-carboxylate with hydrogen chloride in methanol, pyridine-2-carboxylic acid (3RS,4RS)-2-[4-(4-fluoro-phenyl)-piperidin-3-yloxymethyl]-benzylamide dihydrochloride as a white solid; MS: 420 (M+H)⁺;

38) from tert-butyl (3RS,4RS)-3-(3-benzoyl-benzyloxy)-4-(4-fluoro-phenyl)-piperidine-1-carboxylate with hydrogen chloride in methanol, (3RS,4RS)-[3-[4-(4-fluoro-phenyl)-piperidin-3-yloxymethyl]-phenyl]-phenyl-methanone hydrochloride as a white, amorphous solid; MS: 390 (M+H)⁺;

39) from tert-butyl (3RS,4RS)-3-(naphthalen-2-ylmethoxy)-4-[4-[2-(5-phenyl-[1,2,4]oxadiazol-3-yl)-ethyl]-phenyl]-piperidine-1-carboxylate with hydrogen chloride in methanol, (3RS,4RS)-3-(naphthalen-2-ylmethoxy)-4-[4-[2-(5-phenyl-[1,2,4]oxadiazol-3-yl)-ethyl]-phenyl]-piperidine as a white solid; MS: 490 (M+H)⁺;

40) from tert-butyl (3RS,4RS)-3-(naphthalen-2-ylmethoxy)-4-[4-[2-(3-phenyl-[1,2,4]oxadiazol-5-yl)-ethyl]-phenyl]-piperidine-1-carboxylate with hydrogen chloride in methanol, (3RS,4RS)-3-(naphthalen-2-ylmethoxy)-4-[4-[2-(3-phenyl-[1,2,4]oxadiazol-5-yl)-ethyl]-phenyl]-piperidine as a white solid; MS: 490 (M+H)⁺;

41) from tert-butyl (3RS,4RS)-3-(naphthalen-2-ylmethoxy)-4-[4-(3-phenyl-[1,2,4]oxadiazol-5-ylmethoxy)-phenyl]-piperidine-1-carboxylate with trifluoroacetic acid, (3RS,4RS)-3-(naphthalen-2-ylmethoxy)-4-[4-(3-phenyl-[1,2,4]oxadiazol-5-ylmethoxy)-phenyl]-piperidine trifluoroacetate as a white solid; m.p.: 195-196° C.;

42) from tert-butyl (3RS,4RS)-3-(naphthalen-2-ylmethoxy)-4-[4-(3-pyridin-3-yl-[1,2,4]oxadiazol-5-ylmethoxy)-phenyl]-piperidine-1-carboxylate with trifluoroacetic acid, (3RS,4RS)-3-(5-[4-[3-(naphthalen-2-ylmethoxy)-piperidin-4-yl]-phenoxymethyl]-[1,2,4]oxadiazol-3-yl)-pyridine trifluoroacetate as a white solid; MS: 493 (M+H)⁺;

43) from tert-butyl (3RS,4RS)-3-(naphthalen-2-ylmethoxy)-4-[4-(3-phenyl-isoxazol-5-ylmethoxy)-phenyl]-piperidine-1-carboxylate with trifluoroacetic acid, (3RS,4RS)-3-(naphthalen-2-ylmethoxy)-4-[4-(3-phenyl-isoxazol-5-ylmethoxy)-phenyl]-piperidine trifluoroacetate as a white solid; MS: 491 (M+H)⁺;

44) from tert-butyl (3RS,4RS)-4-[4-(3-benzyloxy-propoxy)-phenyl]-3-(3-methoxy-benzyloxy)-piperidine-1-carboxylate by means of hydrogen chloride in methanol, (3RS,4RS)-4-[4-(3-benzyloxy-propoxy)-phenyl]-3-(3-methoxy-benzyloxy)-piperidine hydrochloride as a colorless oil; MS: 462 (M+H)⁺;

45) from tert-butyl (3RS,4RS)-4-[4-(3-benzyloxy-propoxy)-phenyl]-3-[3-(2-trimethylsilanyl-ethoxymethoxy)-benzyloxy]-piperidine-1-carboxylate with hydrogen chloride in methanol with simultaneous cleavage of the SEM group, (3RS,4RS)-3-{4-[4-(3-benzyloxy-propoxy)-phenyl]-piperidin-3-yloxymethyl}-phenol as a colorless oil; MS: 448 (M+H)⁺;

46) from tert-butyl (3RS,4RS)-4-[4-(3-benzyloxy-propoxy)-phenyl]-3-(3-chloro-4-methoxy-benzyloxy)-piperidine-1-carboxylate with zinc bromide in methylene chloride, (3RS,4RS)-4-[4-(3-benzyloxy-propoxy)-phenyl]-3-(3-chloro-4-methoxy-benzyloxy)-piperidine hydrobromide as a colorless oil; MS: 497 (M+H)⁺;

47) from tert-butyl (3RS,4RS)-4-[4-(3-benzyloxy-propoxy)-phenyl]-3-(3,4-dichloro-benzyloxy)-piperidine-1-carboxylate by means of hydrogen chloride in methanol, (3RS,4RS)-4-[4-(3-benzyloxy-propoxy)-phenyl]-3-(3,4-dichloro-benzyloxy)-piperidine hydrochloride as a colorless oil; MS: 500 (M)⁺;

48) from tert-butyl (3RS,4RS)-4-[4-(3-benzyloxy-propoxy)-phenyl]-3-(2,5-dichloro-benzyloxy)-piperidine-1-carboxylate by means of hydrogen chloride in methanol, (3RS,4RS)-4-[4-(3-benzyloxy-propoxy)-phenyl]-3-(2,5-dichloro-benzyloxy)-piperidine hydrochloride as a colorless oil; MS: 500 (M)⁺;

49) from tert-butyl (3RS,4RS)-3-benzyloxy-4-[4-(3-benzyloxy-propoxy)-phenyl]-piperidine-1-carboxylate with zinc bromide in methylene chloride, (3RS,4RS)-3-benzyloxy-4-[4-(3-benzyloxy-propoxy)-phenyl]-piperidine hydrobromide as a colorless solid; MS: 432 (M+H)⁺;

50) from tert-butyl (3RS,4RS)-4-[4-(3-benzyloxy-propoxy)-phenyl]-3-(2,5-dimethyl-benzyloxy)-piperidine-1-carboxylate with zinc bromide in methylene chloride, (3RS,4RS)-4-[4-(3-benzyloxy-propoxy)-phenyl]-3-(2,5-dimethyl-benzyloxy)-piperidine hydrobromide as a colorless oil; MS: 460 (M+H)⁺;

51) from tert-butyl (3RS,4RS)-4-[4-(3-benzyloxy-propoxy)-phenyl]-3-(4-ethyl-benzyloxy)-piperidine-1-carboxylate with zinc bromide in methylene chloride, (3RS,4RS)-4-[4-(3-benzyloxy-propoxy)-phenyl]-3-(4-ethyl-benzyloxy)-piperidine as a colorless oil; MS: 460 (M+H)⁺;

52) from a mixture of tert-butyl (3RS,4RS)-4-[4-(3-benzyloxy-propoxy)-phenyl]-3-(3- and 4-vinyl-benzyloxy)-piperidine-1-carboxylate with zinc bromide in methylene chloride, a mixture of (3RS,4RS)-4-[4-(3-benzyloxy-propoxy)-phenyl]-3-(3 and 4-vinyl-benzyloxy)-piperidine hydrobromide as a colorless solid; MS: 458 (M+H)⁺;

53) from tert-butyl (3RS,4RS)-4-[4-(3-benzyloxy-propoxy)-phenyl]-3-(2,3-dihydro-benzo[11,4]dioxin-6-ylmethoxy)-piperidine-1-carboxylate with zinc bromide in methylene chloride, (3RS,4RS)-4-[4-(3-benzyloxy-propoxy)-phenyl]-3-(2,3-dihydro-benzo[1,4]dioxin-6-ylmethoxy)-piperidine as a colorless resin; MS: 490 (M+H)⁺;

54) from tert-butyl (3RS,4RS)-4-[4-(3-benzyloxy-propoxy)-phenyl]-3-(5,6,7,8-tetrahydro-naphthalen-2-ylmethoxy)-piperidine-1-carboxylate with zinc bromide in methylene chloride, (3RS,4RS)-4-[4-(3-benzyloxy-propoxy)-phenyl]-3-(5,6,7,8-tetrahydro-naphthalen-2-ylmethoxy)-piperidine as a colorless resin; MS: 486 (M+H)⁺;

55) from tert-butyl (3RS,4RS)-4-[4-(3-benzyloxy-propoxy)-phenyl]-3-[4-(2-trimethylsilanyl-ethoxymethoxy)-naphthalen-2-ylmethoxy]-piperidine-1-carboxylate with hydrogen chloride in methanol with simultaneous cleavage of the SEM group, (3RS,4RS)-3-{4-[4-(3-benzyloxy-propoxy)-phenyl]-piperidin-3-yloxymethyl}-naphthalen-1-ol as a colorless resin; MS: 498 (M+H)⁺;

56) from tert-butyl (3RS,4RS)-4-[4-(4-benzyloxy-butoxy)-phenyl]-3-(naphthalen-2-ylmethoxy)-piperidine-1-carboxylate with hydrogen chloride in methanol, (3RS,4RS)-4-[4-(4-benzyloxy-butoxy)-phenyl]-3-(naphthalen-2-ylmethoxy)-piperidine as a colorless oil; MS: 496 (M+H)⁺;

57) from tert-butyl (3SR,4RS,5RS)-4-[4-(2-chloro-benzoyloxy-methyl)-phenyl]-3-methoxymethyl-5-(naphthalen-2-ylmethoxy)-piperazine-1-carboxylate with hydrogen chloride in methanol, (3SR,4RS,5RS)-4-[3-methoxymethyl-5-(naphthalen-2-ylmethoxy)-piperidin-4-yl]-benzyl 2-chloro-benzoate as a colorless foam; MS: 530 (M+H)⁺;

58) from tert-butyl (3SR,4RS,5RS)-4-(4-methoxycarbonyl-phenyl)-3-methoxymethyl-5-(naphthalen-2-ylmethoxy)-piperidine-1-carboxylate with hydrogen chloride in methanol, methyl (3SR,4RS,5RS)-4-[3-methoxymethyl-5-naphthalen-2-ylmethoxy-piperidin-4-yl]-benzoate as a colorless solid; MS: 420 (M+H)⁺;

59) from tert-butyl (3SR,4RS,5RS)-4-(4-benzyloxymethyl-phenyl)-3-methoxymethyl-5-(naphthalen-2-ylmethoxy)-piperidine-1-carboxylate with hydrogen chloride in methanol, (3SR,4RS,5RS)-4-(4-benzyloxymethyl-phenyl)-3-methoxymethyl-5-(naphthalen-2-ylmethoxy)-piperidine as a colorless foam; MS: 482 (M+H)⁺;

60) from tert-butyl (1RS,2RS,3RS,5SR)-3-[4-(2-benzyloxy-propoxymethyl)-phenyl]-2-(naphthalen-2-ylmethoxy)-8-aza-bicyclo[3.2.1]octane-8-carboxylate with hydrogen chloride in methanol, (1RS,2RS,3RS,5SR)-2-(naphthalen-2-ylmethoxy)-3-[4-(3-phenoxy-propoxymethyl)phenyl]-8-azabicyclo[3.2.1]octane as a colorless oil; MS: 508 (M+H)⁺.

The BOC derivatives used as starting materials were prepared as follows:

(a) In an analogous manner to that described in Example 44 (e), by alkylating tert-butyl (3RS,4RS)-3-hydroxy-4-(4-hydroxy-phenyl)-piperidine-1-carboxylate [Example 46 (b)] with propargyl bromide in the presence of potassium carbonate in acetone there was obtained tert-butyl (3RS,4RS)-3-hydroxy-4-(4-prop-2-ynyloxy-phenyl)-piperidine-1-carboxylate, alkylation of which with 2-bromomethylnaphthalene in analogy to Example 1 (g) gave tert-butyl (3RS,4RS)-3-(naphthalen-2-ylmethoxy)-4-(4-prop-2-ynyloxy-phenyl)-piperidine-1-carboxylate as a pale yellow solid; MS: 472 (M+H)⁺.

(b) In an analogous manner to that described in Example 44 (e), by alkylating tert-butyl (3RS,4RS)-3-hydroxy-4-(4-hydroxy-phenyl)-piperidine-1-carboxylate [Example 46 (b)] with allyl bromide in the presence of potassium carbonate in acetone there was obtained tert-butyl (3RS,4RS)-4-(4-allyloxy-phenyl)-3-hydroxy-piperidine-1-carboxylate, alkylation of which with 2-bromomethylnaphthalene in analogy to Example 1 (g) gave tert-butyl (3RS,4RS)-4-(4-allyloxy-phenyl)-3-(naphthalen-2-ylmethoxy)-piperidine-1-carboxylate as a colorless resin; MS: 474 (M+H)⁺.

(c) In an analogous manner to that described in Example 1 (g), by alkylating tert-butyl (3RS,4RS)-3-hydroxy-4-(4-hydroxy-phenyl)-piperidine-1-carboxylate [Example 46 (b)] with (RS)-2,2-dimethyl-[1,3]dioxolan-4-ylmethylmethanesulfonate in the presence of sodium hydride there was obtained a mixture of tert-butyl (3RS,4RS)- and (3SR,4SR)-4-[4-[(RS)-2,2-dimethyl-[1,3]dioxolan-4-ylmethoxy]-phenyl]-3-hydroxy-piperidine-1-carboxylate, alkylation of which with 2-bromomethylnaphthalene in analogy to Example 1 (g) gave a mixture of tert-butyl (3RS,4RS)- and (3SR,4SR)-4-[4-[(RS)-2,2-dimethyl-[1,3]dioxolan-4-ylmethoxy]-phenyl]-3-(naphthalen-2-ylmethoxy)-piperidine-1-carboxylate as a white solid; MS: 547 (M)⁺.

(d) In an analogous manner to that described in Example 1 (9), by alkylating tert-butyl (3RS,4RS)-3-hydroxy-4-(4-hydroxy-phenyl)-piperidine-1-carboxylate [Example 46 (b)] with (RS)-2,3-epoxypropyl p-toluenesulfonate in the presence of sodium hydride there was obtained a mixture of tert-butyl (3RS,4RS)- and (3SR,4SR)-3-hydroxy-4-[(RS)-4-oxiranylmethoxy-phenyl]-piperidine-1-carboxylate, alkylation of which with 2-bromomethylnaphthalene in analogy to Example 1 (g) gave a mixture of tert-butyl (3RS,4RS)- and (3SR,4SR)-3-(naphthalen-2-ylmethoxy)-4-[(RS)-4-oxiranylmethoxy-phenyl]-piperidine-1-carboxylate. Subsequent epoxide opening with potassium phenolate in analogy to Example 71 (a) yielded a mixture of tert-butyl (3RS,4RS)- and (3SR,4SR)-4-[4-[(RS)-2-hydroxy-3-phenoxy-propoxy]-phenyl]-3-(naphthalen-2-ylmethoxy)-piperidine-1-carboxylate as a white solid; MS: 584 (M+H)⁺.

(e) Epoxide opening of a mixture of tert-butyl (3RS,4RS)- and (3SR,4SR)-3-(naphthalen-2-ylmethoxy)-4-[(RS)-4-oxiranylmethoxy-phenyl]-piperidine-1-carboxylate [Example 86 (d)] with sodium methylate in N,N-dimethylformamide yielded a mixture of tert-butyl (3RS,4RS)- and (3SR,4SR)-4-[4-[(RS)-2-hydroxy-3-methoxy-propoxy]-phenyl]-3-(naphthalen-2-ylmethoxy)-piperidine-1-carboxylate, alkylation of which with benzyl bromide analogously to Example 1 (g) gave a mixture of tert-butyl (3RS,4RS)- and (3SR,4SR)-4-[4-[(RS)-2-benzyloxy-3-methoxy-propoxy]-phenyl]-3-(naphthalen-2-ylmethoxy)-piperidine-1-carboxylate as a colorless resin; MS: 612 (M+H)⁺.

(f) Epoxide opening of a mixture of tert-butyl (3RS,4RS)- and (3SR,4SR)-3-(naphthalen-2-ylmethoxy)-4-[(RS)-4-oxiranylmethoxy-phenyl]-piperidine-1-carboxylate (Example 86 (d)] with sodium thiophenolate in analogy to Example 71 (a) gave a mixture of tert-butyl 3RS,4RS)- and (3SR,4SR)-4-[4-[(RS)-2-hydroxy-3-phenylsulfanyl-propoxy]-phenyl]-3-(naphthalen-2-ylmethoxy)-piperidine-1-carboxylate as a white solid; MS: 600 (M+H)⁺.

(g) Alkylation of a mixture of tert-butyl (3RS,4RS)- and (3SR,4SR)-4-[4-[(RS)-2-hydroxy-3-phenoxy-propoxy]-phenyl]-3-(naphthalen-2-ylmethoxy)-piperidine-1-carboxylate [Example 86 (d)] with methyl iodide in analogy to Example 1 (g) yielded a mixture of tert-butyl (3RS,4RS)- and (3SR,4SR)-4-[4-[(RS)-2-methoxy-3-phenoxy-propoxy)-phenyl]-3-(naphthalen-2-ylmethoxy)-piperidine-1-carboxylate as a colorless resin; MS: 598 (M+H)⁺.

(h) In analogy to Example 22 (k), by benzoylating a mixture of tert-butyl (3RS,4RS)- and (3SR,4SR)-4-[4-[(RS)-2-hydroxy-3-methoxy-propoxy]-phenyl]-3-(naphthalen-2-ylmethoxy)-piperidine-1-carboxylate [Example 86 (e)] there was obtained a mixture of tert-butyl (3RS,4RS)- and (3SR,4SR)-4-[4-[(RS)-2-benzoyloxy-3-methoxy-propoxy]-phenyl]-3-(naphthalen-2-ylmethoxy)-piperidine-1-carboxylate as a colorless resin; MS: 626 (M+H)⁺.

(i) Epoxide opening of a mixture of tert-butyl (3RS,4RS)- and (3SR,4SR)-3-(naphthalen-2-ylmethoxy)-4-[(RS)-4-oxiranylmethoxy-phenyl]-piperidine-1-carboxylate [Example 86 (d)] with sodium benzylate in N,N-dimethylformamide gave a mixture of tert-butyl (3RS,4RS)- and (3SR,4SR)-4-[4-[(RS)-3-Benzyloxy-2-hydroxy-propoxy]-phenyl]-3-(naphthalen-2-ylmethoxy)-piperidine-1-carboxylate, alkylation of which with methyl iodide analogously to Example 1 (g) yielded a mixture of tert-butyl (3RS,4RS)- and (3SR,4SR)-4-[4-[(RS)-3-benzyloxy-2-methoxypropoxy]-phenyl]-3-(naphthalen-2-ylmethoxy)-piperidine-1-carboxylate as a colorless resin; MS: 612 (M+H)⁺.

(j) In an analogous manner to that described in Example 1 (g) by alkylating tert-butyl (3RS,4RS)-4-(4-(2-hydroxy-ethoxy)-phenyl]-3-(naphthalen-2-ylmethoxy)-piperidine-1-carboxylate [Example 53 (c)] with 3-chloromethyl-pyridine there was obtained tert-butyl (3RS,4RS)-3-(naphthalen-2-ylmethoxy)-4-{4-[2-(pyridin-3-ylmethoxy)-ethoxy]-phenyl}-piperidine-1-carboxylate; MS: 569 (M+H)⁺.

(k) In an analogous manner to that described in Example 1 (g), by alkylating tert-butyl (3RS,4RS)-4-[4-(2-hydroxy-ethoxy)-phenyl]-3-[4-(2-trimethylsilanyl-ethoxy)-naphthalen-2-ylmethoxy]-piperidine-1-carboxylate [Example 55 (b)] with 3-chloromethyl-pyridine there was obtained tert-butyl (3RS,4RS)-4-{4-[2-(pyridin-3-ylmethoxy)-ethoxy]-phenyl}-3-[4-(2-trimethylsilanyl-ethoxymethoxy)-naphthalen-2-ylmethoxy]-piperidine-1-carboxylate.

(l) In an analogous manner to that described in Example 1 (g), by alkylating tert-butyl (3RS,4RS)-4-[4-(2-hydroxy-ethoxy)-phenyl]-3-(naphthalen-2-ylmethoxy)-piperidine-1-carboxylate [Example 53 (c)] with 4-chloromethyl-pyridine there was obtained tert-butyl (3RS,4RS)-3-(naphthalen-2-ylmethoxy)-4-{4-[2-(pyridin-4-ylmethoxy)-ethoxy)-phenyl}-piperidine-1-carboxylate as a colorless oil.

(m) In an analogous manner to that described in Example 1 (g), by alkylating tert-butyl (3RS,4RS)-4-[4-(2-hydroxy-ethoxy)-phenyl]-3-(naphthalen-2-ylmethoxy)-piperidine-1-carboxylate [Example 53 (c)] with 2-chloromethyl-pyridine there was obtained tert-butyl (3RS,4RS)-3-(naphthalen-2-ylmethoxy)-4-{4-[2-(pyridin-2-ylmethoxy)-ethoxy]-phenyl}-piperidine-1-carboxylate as a colorless oil.

(n) In an analogous manner to that described in Example 44 (e), by alkylating tert-butyl (3RS,4RS)-3-hydroxy-4-(4-hydroxy-phenyl)-piperidine-1-carboxylate [Example 46 (b)] with benzyl 3-bromopropyl ether in the presence of potassium carbonate in butan-2-one there was obtained tert-butyl (3RS,4RS)-4-[4-(3-benzyloxy-propoxy)-phenyl]-3-hydroxy-piperidine-1-carboxylate, alkylation of which with 2-bromomethyl-naphthalene analogously to Example 1 (g) gave tert-butyl (3RS,4RS)-4-[4-(3-benzyloxy-propoxy)-phenyl]-3-(naphthalen-2-ylmethoxy)-piperidine-1-carboxylate as a colorless oil; MS: 582 (M+H)⁺.

(o) In an analogous manner to that described in Example 1 (g), by alkylating tert-butyl (3RS,4RS)-4-[4-(3-hydroxy-propoxy)-phenyl]-3-(naphthalen-2-ylmethoxy)-piperidine-1-carboxylate [Example 57 (c)] with 2-chloromethyl-pyridine there was obtained tert-butyl (3RS,4RS)-3-(naphthalen-2-ylmethoxy)-4-[4-[3-(pyridin-2-ylmethoxy)-propoxy]-phenyl]-piperidine-1-carboxylate as a colorless resin; MS: 583 (M+H)⁺.

(p) In an analogous manner to that described in Example 1 (g), by alkylating tert-butyl (3RS,4RS)-4-[4-(3-hydroxy-propyl)-phenyl]-3-(naphthalen-2-ylmethoxy)-piperidine-1-carboxylate [Example 24 (t)] with 2-chloromethyl-pyridine there was obtained tert-butyl (3RS,4RS)-3-(naphthalen-2-ylmethoxy)-4-[4-[3-(pyridin-2-ylmethoxy)-propyl]-phenyl]-piperidine-1-carboxylate as a colorless, amorphous solid, which was used in the following step without further purification and characterization.

(q) In an analogous manner to that described in Example 44 (e), by alkylating tert-butyl (3RS,4RS)-4-[4-(3-methylamino-propoxy)-phenyl]-3-(naphthalen-2-ylmethoxy)-piperidine-1-carboxylate [Example 57 (e)] with benzyl bromide there was obtained tert-butyl (3RS,4RS)-4-[4-[3-(benzyl-methyl-amino)-propoxy]-phenyl]-3-(naphthalen-2-ylmethoxy)-piperidine-1-carboxylate as a colorless solid; MS: 595 (M+H)⁺.

(r) In an analogous manner to that described in Example 1 (g), by alkylating a mixture of tert-butyl (3RS,4RS)- and (3SR,4SR)-3-hydroxy-4-[4-[3-[(RS)-tetrahydro-pyran-2-yloxy]-propoxy]-phenyl]-piperidine-1-carboxylate [Example 57 (a)] with 1-methoxy-2-bromomethyl-naphthalene [Example 7 (f)] there was obtained a mixture of tert-butyl (3RS,4RS)- and (3SR,4SR)-3-(1-methoxy-naphthalen-2-ylmethoxy)-4-[4-[3-[(RS)-tetrahydro-pyran-2-yloxy]-propoxy]-phenyl]-piperidine-1-carboxylate. Cleavage of the THP group by means of pyridinium (toluene-4-sulfonate) in ethanol analogously to Example 53 (c) gave tert-butyl (3RS,4RS)-4-[4-(3-hydroxy-propoxy)-phenyl]-3-(1-methoxy-naphthalen-2-ylmethoxy)-piperidine-1-carboxylate, reaction of which with bis-(benzothiazol-2-yl) disulfide analogously to Example 33 (a) gave tert-butyl (3RS,4RS)-4-[4-[3-(benzothiazol-2-ylsulfonyl)-propoxy]-phenyl]-3-(1-methoxy-naphthalen-2-ylmethoxy)-piperidine-1-carboxylate as a colorless liquid; MS: 671 (M+H)⁺.

(s) In an analogous manner to that described in Example 33 (a), by reacting tert-butyl (3RS,4RS)-4-[4-(3-hydroxy-propyl)-phenyl]-3-(naphthalen-2-ylmethoxy)-piperidine-1-carboxylate [Example 24 (t)] with diphenyl disulfide there was obtained tert-butyl (3RS,4RS)-3-(naphthalen-2-ylmethoxy)-4-[4-(3-phenylsulfanyl-propyl)-phenyl]-piperidine-1-carboxylate as a colorless, amorphous solid; MS: 568 (M+H)⁺.

(t) In an analogous manner to that described in Example 33 (a), by reacting tert-butyl (3RS,4RS)-4-[4-(3-hydroxy-propyl)-phenyl]-3-(naphthalen-2-ylmethoxy)-piperidine-1-carboxylate [Example 24 (t)] with bis-(benzothiazol-2-yl) disulfide there was obtained tert-butyl (3RS,4RS)-4-{4-[3-(benzothiazol-2-ylsulfanyl}-propyl]-phenyl]-3-(naphthalen-2-ylmethoxy)-piperidine-1-carboxylate, which was used in the following step without further purification and characterization.

(u) (α) In an analogous manner to that described in Example 1 (g), by alkylating tert-butyl (3RS,4RS)-3-hydroxy-4-[4-(2-trityloxy-ethyl)-phenyl]-piperidine-1-carboxylate [Example 29 (t)] with 3-chloromethyl-1-(2-trimethylsilanyl-ethoxymethoxy)-naphthalene [Example 5 (c)] there was obtained tert-butyl (3RS,4RS)-3-[4-(2-trimethylsilanyl-ethoxymethoxy)-naphthalen-2-ylmethoxy]-4-[4-(2-trityloxy-ethyl)-phenyl]-piperidine-1-carboxylate.

(β) Selective cleavage of the trityl group was effected analogously to the procedure published by E. Krainer et al. in Tetrahedron Letters 1993, 1713-1716 by treating a solution of 780 mg (0.92 mmol) of tert-butyl (3RS,4RS)-3-[4-(2-trimethylsilanyl-ethoxymethoxy)-naphthalen-2-ylmethoxy]-4-[4-(2-trityloxy-ethyl)-phenyl]-piperidine-1-carboxylate in 15 ml of methylene chloride with a solution of 436 mg (3.68 mmol) of trifluoroacetic acid and 803 mg (3.68 mmol) of trifluoroacetic anhydride in 2 ml of methylene chloride. After 30 seconds the reaction mixture was cooled to 0° C. and treated with 4 ml of triethylamine. After 5 minutes 10 ml of methanol were added and the mixture was stirred for 10 minutes. Subsequently, the mixture was washed with saturated sodium hydrogen carbonate solution and the aqueous phase was thereafter back-extracted with 10 ml of methylene chloride. The combined organic phases were dried over sodium sulfate and then evaporated under reduced pressure. The crude product was chromatographed on silica gel using a 4:1 mixture of hexane and ethyl acetate as the eluent. There were obtained 553 mg of tert-butyl (3RS,4RS)-4-[4-(2-hydroxy-ethyl)-phenyl]-3-[4-(2-trimethylsilanyl-ethoxy-methoxy)-naphthalen-2-ylmethoxy]-piperidine-1-carboxylate as a colorless oil.

(γ) In an analogous manner to that described in Example 34, from tert-butyl (3RS,4RS)-4-[4-(2-hydroxy-ethyl)-phenyl]-3-[4-(2-trimethylsilanyl-ethoxymethoxy)-naphthalen-2-ylmethoxy]-piperidine-1-carboxylate via the corresponding mesylate by reactions with 2-mercaptopyrimidine there was obtained tert-butyl (3RS,4RS)-4-{4-[2-(pyrimidin-2-ylsulfanyl)-ethyl]-phenyl)-3-[4-(2-trimethylsilanyl-ethoxymethoxy)-naphthalen-2-ylmethoxy]-piperidine-1-carboxylate as a colorless oil, which was used in the following step without further purification and characterization.

(v) In an analogous manner to that described in Example 33 (a), from tert-butyl (3RS,4RS)-4-[4-(2-hydroxy-ethyl)-phenyl]-3-[4-(2-trimethylsilanyl-ethoxymethoxy)-naphthalen-2-ylmethoxy]-piperidine-1-carboxylate by reaction with 2,2′-dithiopyridine there was obtained tert-butyl (3RS,4RS)-4-{4-[2-(pyridin-2-ylsulfanyl)-ethyl]-phenyl}-3-[4-(2-trimethylsilanyl-ethoxymethoxy)-naphthalen-2-ylmethoxy]-piperidine-1-carboxylate as a colorless oil, which was used in the following step without further purification and characterization.

(w) In an analogous manner to that described in Example 33 (a), from tert-butyl (3RS,4RS)-4-[4-(2-hydroxy-ethyl)-phenyl]-3-[4-(2-trimethylsilanyl-ethoxymethoxy)-naphthalen-2-ylmethoxy]-piperidine-1-carboxylate by reaction with bis-(benzothiazol-2-yl) disulfide there was obtained tert-butyl (3RS,4RS)-4-[4-[2-(benzothiazol-2-ylsulfanyl)-ethyl]-phenyl]-3-[4-(2-trim ethyl-silanyl-ethoxymethoxy)-naphthalen-2-ylmethoxy]-piperidine-1-carboxylate as a colorless foam; MS: 757 (M+H)⁺.

(x) In an analogous manner to that described in Example 1 (g), by alkylating tert-butyl (3RS,4RS)-4-(4-hydroxymethyl-phenyl)-3-(naphthalen-2-ylmethoxy)-piperidine-1-carboxylate [Example 22 (j)] with 3-chloromethyl-pyridine there was obtained tert-butyl (3RS,4RS)-3-(naphthalen-2-ylmethoxy)-4-[4-(pyridin-3-yl-methoxymethyl)-phenyl)-piperidine-1-carboxylate as a colorless oil; MS: 539 (M+H)⁺.

(y) In an analogous manner to that described in Example 9 (c), by esterifying tert-butyl (3RS,4RS)-4-[4-(2-hydroxy-ethoxy)-phenyl]-3-(naphthalen-2-ylmethoxy)-piperidine-1-carboxylate [Example 53 (c)] with (RS)-2-(4-fluoro-phenyl)-3-methyl-butyric acid (DE 2365555) in the presence of EDC there was obtained a mixture of tert-butyl (3RS,4RS)- and (3SR,4SR)-4-(4-2-[(RS)-2-(4-fluoro-phenyl)-3-methyl-butyryloxy]-ethoxy]-phenyl)-3-(naphthalen-2-ylmethoxy)-piperidine-1-carboxylate as a colorless solid; MS: 656 (M+H)⁺.

(z) In an analogous manner to that described in Example 9 (c), by esterifying tert-butyl (3RS,4RS)-4-[4-(2-hydroxy-ethoxy)-phenyl]-3-(naphthalen-2-ylmethoxy)-piperidine-1-carboxylate [Example 53 (c)] with 1-methyl-pyrrole-2-carboxylic acid in the presence of EDC there was obtained tert-butyl (3RS,4RS)-4-[4-[2-(1-methyl-1H-pyrrol-2-carbonyloxy)-ethoxy]-phenyl]-3-(naphthalen-2-ylmethoxy)-piperidine-1-carboxylate as a colorless oil; MS: 585 (M+H)⁺.

(aa) In an analogous manner to that described in Example 22 (k), by acylating tert-butyl (3RS,4RS)-4-[4-(3-hydroxy-propyl)-phenyl]-3-(naphthalen-2-ylmethoxy)-piperidine-1-carboxylate [Example 24 (t)] with benzoyl chloride there was obtained tert-butyl (3RS,4RS)-4-[4-(3-benzoyloxy-propyl)-phenyl]-3-(naphthalen-2-ylmethoxy)-piperidine-1-carboxylate as an almost colorless solid; MS: 580 (M+H)⁺.

(bb) In an analogous manner to that described in Example 22 (k), by acylating tert-butyl (3RS,4RS)-4-[4-(3-hydroxy-propyl)-phenyl]-3-(naphthalen-2-ylmethoxy)-piperidine-1-carboxylate [Example 24 (t)] with 3-methoxy-benzoyl chloride there was obtained tert-butyl (3RS,4RS)-4-{4-[3-(3-Methoxy-benzoyloxy)-propyl]-phenyl}-3-(naphthalen-2-ylmethoxy)-piperidine-1-carboxylate as a colorless, amorphous solid; MS: 610 (M+H)⁺.

(cc) In an analogous manner to that described in Example 1 (g), by alkylating tert-butyl (3RS,4RS)-3-hydroxy-4-(4-trityloxymethyl-phenyl)-piperidine-1-carboxylate [Example 22 (h)] with 2-chloromethyl-1-(2-trimethylsilanyl-ethoxymethoxy)-naphthalene [Example 6 (c)] there was obtained tert-butyl (3RS,4RS)-3-[1-(2-trimethylsilanyl-ethoxymethoxy)-naphthalen-2-ylmethoxy]-4-(4-trityloxymethyl-phenyl)-piperidine-1-carboxylate. Selective cleavage of the trityl group analogously to Example 86 (u) (β) gave tert-butyl (3RS,4RS)-4-(4-hydroxymethyl-phenyl)-3-[1-(2-trimethylsilanyl-ethoxymethoxy)naphthalen-2-ylmethoxy]-piperidine-1-carboxylate, acylation of which with 3-methoxy-benzoyl chloride analogously to Example 22 (k) gave tert-butyl (3RS,4RS)-4-[4-(3-methoxy-benzoyloxymethyl)-phenyl]-3-[1-(2-trimethylsilanyl-ethoxymethoxy)-naphthalen-2-ylmethoxy]-piperidine-1-carboxylate as a colorless oil, MS: 745 (M+NH₄)⁺.

(dd) In an analogous manner to that described in Example 44 (e), by alkylating tert-butyl (3RS,4RS)-4-(4-hydroxy-phenyl)-3-(naphthalen-2-ylmethoxy)-piperidine-1-carboxylate [Example 61 (c)] with ethyl bromoacetate there was obtained tert-butyl (3RS,4RS)-4-(4-ethoxycarbonylmethoxy-phenyl)-3-(naphthalen-2-ylmethoxy)-piperidine-1-carboxylate as a colorless solid; MS: 520 (M+H)⁺.

(ee) Saponification of tert-butyl (3RS,4RS)-4-(4-ethoxycarbonyl-methoxy-phenyl)-3-(naphthalen-2-ylmethoxy)-piperidine-1-carboxylate [Example 86 (dd)] with 1 N sodium hydroxide in methanol yielded tert-butyl (3RS,4RS)-4-(4-carboxymethoxy-phenyl)-3-(naphthalen-2-ylmethoxy)-piperidine-1-carboxylate, condensation of which with benzylamine in the presence of HBTU analogously to Example 36 (b) gave tert-butyl (3RS,4RS)-4-[4-(benzylcarbamoyl-methoxy)-phenyl]-3-(naphthalen-2-ylmethoxy)-piperidine-1-carboxylate as a yellowish solid; MS: 598 (M+NH₄)⁺.

(ff) In an analogous manner to that described in Example 24 (m), by reacting tert-butyl (3RS,4RS)-4-(4-hydroxy-phenyl)-3-(naphthalen-2-ylmethoxy)-piperidine-1-carboxylate with 2-pyridyl isocyanate there was obtained tert-butyl (3RS,4RS)-3-(naphthalen-2-ylmethoxy)-4-[4-(pyridin-2-ylcarbamoyloxy)-phenyl]-piperidine-1-carboxylate as a white solid; MS: 554 (M+H)⁺.

(gg) In an analogous manner to that described in Example 68 (b), by Swern oxidation of a mixture of tert-butyl (3RS,4RS)- and (3SR,4SR)-4-[4-[(RS)-2-hydroxy-3-phenoxy-propoxy]-phenyl]-3-(naphthalen-2-ylmethoxy)-piperidine-1-carboxylate there was obtained tert-butyl (3RS,4RS)-3-(naphthalen-2-ylmethoxy)-4-[4-(2-oxo-3-phenoxypropoxy)-phenyl]-piperidine-1-carboxylate as a white solid; MS: 582 (M+H)⁺.

(hh) (α) A solution of 5 mg (0.04 mmol) of potassium bromide and 20 mg (0.24 mmol) of sodium hydrogen carbonate in 10 ml of water was added at room temperature and while stirring to a solution of 0.270 g (0.58 mmol) of tert-butyl (3RS,4RS)-4-[4-(2-hydroxy-ethyl)-phenyl]-3-naphthalen-2-ylmethoxy-piperidine-1-carboxylate [Example 29(h)] in 10 ml of methylene chloride under argon. The V reaction mixture was cooled to 0° C. and treated with 2 mg (0.01 mmol) of 2,2,6,6-tetramethyl-1-piperidinyloxyl (TEMPO). Then, 1 ml (0.658 mmol) of Javelle water was sprayed into the reaction mixture while stirring continuously. After the addition the reaction mixture was stirred at 0° C. for about 30 minutes. For the working-up, 20 ml of a 1:1 mixture of methylene chloride and water were added, the reaction mixture was washed with 10 ml of saturated sodium chloride solution and the aqueous phase was back-extracted with 10 ml of methylene chloride. The combined organic phases were dried over sodium sulfate and evaporated under reduced pressure. The crude product was purified by flash chromatography on silica gel with a 2:1 mixture of hexane and ethyl acetate as the eluent. There were obtained 200 mg (75% of theory) of tert-butyl (3RS,4RS)-3-(naphthalen-2-ylmethoxy)-4-[4-(2-oxo-ethyl)-phenyl]-piperidine-1-carboxylate as a foam; MS: 460 (M+H)⁺.

(β) In an analogous manner to that described in Example 40 (a), by a Grignard reaction of tert-butyl (3RS,4RS)-3-(naphthalen-2-ylmethoxy)-4-[4-(2-oxo-ethyl)-phenyl]-piperidine-1-carboxylate with phenylmagnesium chloride there was obtained a mixture of tert-butyl (3RS,4RS)-4-[4-[(RS)-2- and [(SR)-2-hydroxy-2-phenyl-ethyl]-phenyl]-3-(naphthalen-2-ylmethoxy)-piperidine-1-carboxylate as a colorless oil.

(ii) In an analogous manner to that described in Example 9 (a)-(c), by alkylating tert-butyl (3RS,4RS)-4-(4-fluoro-phenyl)-3-hydroxy-piperidine-1-carboxylate [Example 3 (b)] with 2-bromomethylbenzonitrile there was obtained tert-butyl (3RS,4RS)-3-(2-cyano-benzyloxy)-4-(4-fluoro-phenyl)-piperidine-1-carboxylate, reduction of which using borane-dimethyl sulfide complex gave tert-butyl (3RS,4RS)-3-(2-aminomethyl-benzyloxy)-4-(4-fluoro-phenyl)-piperidine-1-carboxylate. Subsequent acylation with pyridine-2-carboxylic acid in the presence of EDC yielded tert-butyl (3RS,4RS)-4-(4-fluoro-phenyl)-3-(2-{[(pyridine-2-carbonyl)-amino]-methyl}-benzyloxy)-piperidine-1-carboxylate as a white solid.

(jj) In an analogous manner to that described in Example 1 (g), by alkylating tert-butyl (3RS,4RS)-4-(4-fluoro-phenyl)-3-hydroxy-piperidine-1-carboxylate [Example 3 (b)] with (3-bromomethyl-phenyl)-phenyl-methanone [J. Med. Chem. 1984, 27 (12), 1682-1690] there was obtained tert-butyl (3RS,4RS)-3-(3-benzoyl-benzyloxy)-4-(4-fluoro-phenyl)-piperidine-1-carboxylate as a yellowish liquid; MS: 490 (M+H)⁺.

(kk) (α) A solution of 470 mg (1.0 mmol) of tert-butyl (3RS,4RS)-4-[4-(2-cyano-ethyl)-phenyl]-3-naphthalen-2-ylmethoxy-piperidine-1-carboxylate [Example 35 (b)] and 348 mg (5.0 mmol) of hydroxylamine hydrochloride in 6 ml of a 1 M sodium methylate solution in methanol was stirred at 65° C. for 5 hours. For working-up, the mixture was partitioned between 40 ml of ethyl acetate and 40 ml of water and thereafter the organic phase was separated. The aqueous phase was extracted twice with 40 ml of ethyl acetate each time. The combined organic phases were dried over magnesium sulfate and finally the solvent was distilled off under reduced pressure. The crude product (550 mg) was purified by chromatography on silica gel with a 20:1:0.1 mixture of methylene chloride, methanol and 28% ammonia solution. There were obtained 501 mg (99% of theory) of tert-butyl (3RS,4RS)-4-{4-[2-(N-hydroxycarbamimidoyl)-ethyl]-phenyl}-3-(naphthalen-2-ylmethoxy)-piperidine-1-carboxylate as a yellow oil, which was used directly in the next step.

(b) In an analogous manner to that described in Example 38, by condensing tert-butyl (3RS,4RS)-4-{4-[2-(N-hydroxy-carbamimidoyl)-ethyl]-phenyl}-3-(naphthalen-2-ylmethoxy)-piperidine-1-carboxylate with benzoic acid in the presence of EDC and subsequent cyclization there was obtained tert-butyl (3RS,4RS)-3-(naphthalen-2-ylmethoxy)-4-[4-[2-(5-phenyl-[1,2,4]oxadiazol-3-yl)-ethyl]-phenyl]-piperidine-1-carboxylate; MS: 590 (M+H)⁺.

(ll) By alkaline saponification of tert-butyl (3RS,4RS)-4-[4-(2-ethoxycarbonyl-ethyl)-phenyl]-3-(naphthalen-2-ylmethoxy)-piperidine-1-carboxylate [Example 24 (s)] with aqueous sodium hydroxide in tetrahydrofuran there was obtained tert-butyl (3RS,4RS)-4-[4-(2-carboxy-ethyl)-phenyl]-3-(naphthalen-2-ylmethoxy)piperidine-1-carboxylate, condensation of which with N-hydroxy-benzamidine in the presence of EDC in an analogous manner to that described in Example 38 gave the corresponding N-hydroxy-benzamidine ester and cyclization of the latter yielded tert-butyl (3RS,4RS)-3-(naphthalen-2-ylmethoxy)-4-[4-[2-(3-phenyl-[1,2,4]oxadiazol-5-yl)-ethyl]-phenyl]-piperidine-1-carboxylate as a yellowish, amorphous solid; MS: 590 (M+H)⁺.

(mm) Saponification of tert-butyl (3RS,4RS)-4-(4-ethoxy-carbonylmethoxy-phenyl)-3-(naphthalen-2-ylmethoxy)-piperidine-1-carboxylate [Example 86 (dd)] with 1 N sodium hydroxide in methanol yielded tert-butyl (3RS,4RS)-4-(4-carboxymethoxy-phenyl)-3-(naphthalen-2-ylmethoxy)-piperidine-1-carboxylate, condensation of which with N-hydroxy-benzamidine in the presence of HBTU in an analogous manner to that described in Example 38 gave the corresponding N-hydroxy-benzamidine ester and cyclization of the latter gave tert-butyl (3RS,4RS)-3-(naphthalen-2-ylmethoxy)-4-[4-(3-phenyl-[1,2,4]oxadiazol-5-ylmethoxy)-phenyl]-piperidine-1-carboxylate as a colorless oil; MS: 592 (M+H)⁺.

(nn) In an analogous manner to that described in Example 38, by condensing tert-butyl (3RS,4RS)-4-(4-carboxymethoxy-phenyl)-3-(naphthalen-2-ylmethoxy)-piperidine-1-carboxylate [Example 86 (mm)] with 3-pyridinamidoxime in the presence of HBTU and subsequent cyclization there was obtained tert-butyl (3RS,4RS)-3-(naphthalen-2-ylmethoxy)-4-[4-(3-pyridin-3-yl-[1,2,4]oxadiazol-5-ylmethoxy)-phenyl]-piperidine-1-carboxylate as a yellowish solid; MS: 593 (M+H)⁺.

(oo) In an analogous manner to that described in Example 44 (e), by alkylating tert-butyl (3RS,4RS)-4-(4-hydroxy-phenyl)-3-(naphthalen-2-ylmethoxy)-piperidine-1-carboxylate [Example 61 (c)] with 5-hydroxymethyl-3-phenyl-4,5-dihydro-isoxazole mesylate, prepared according to a generally known procedure, there was obtained tert-butyl (3RS,4RS)-3-(naphthalen-2-ylmethoxy)-4-[4-(3-phenyl-isoxazol-5-ylmethoxy)-phenyl]-piperidine-1-carboxylate as a colorless solid; MS: 591 (M+H)⁺.

The following BOC derivatives were obtained in an analogous manner to that described in Example 1 (g) by alkylating tert-butyl (3RS,4RS)-4-[4-(3-benzyloxy-propoxy)-phenyl]-3-hydroxy-piperidine-1-carboxylate [Example 86 (n)] as follows:

(pp) with 3-methoxybenzyl chloride to give tert-butyl (3RS,4RS)-4-[4-(3-benzyloxy-propoxy)-phenyl]-3-(3-methoxy-benzyloxy)-piperidine-1-carboxylate, which was obtained as a colorless oil; MS: 579 (M+NH₄)⁺.

(qq) with 1-chloromethyl-3-(2-trimethylsilanyl-ethoxymethoxy)-benzene to give tert-butyl (3RS,4RS)-4-[4-(3-benzyloxy-propoxy)-phenyl]-3-[3-(2-trimethylsilanyl-ethoxymethoxy)-benzyloxy]-piperidine-1-carboxylate, which was obtained as a colorless oil; MS: 695 (M+NH₄)⁺.

The 1-chloromethyl-3-(2-trimethylsilanyl-ethoxymethoxy)-benzene used as the alkylating agent was prepared in analogy to Example 5 (a)-(d) by converting methyl 3-hydroxybenzoate into methyl 3-(2-trimethylsilanyl-ethoxymethoxy)-benzoate by introduction of the SEM group. Subsequent reduction with lithium aluminium hydride gave [3-(2-trimethylsilanylethoxy-methoxy)-phenyl]-methanol and chlorination of the latter gave 1-chloromethyl-3-(2-trimethylsilanyl-ethoxymethoxy)-benzene as a colorless oil; MS: 272 (M)⁺.

(rr) with 3-chloro-4-methoxy-benzyl chloride to give tert-butyl (3RS,4RS)-4-[4-(3-benzyloxy-propoxy)-phenyl]-3-(3-chloro-4-methoxy-benzyloxy)-piperidine-1-carboxylate, which was used in the following step without further purification and characterization;

(ss) with 3,4-dichloro-benzyl chloride to give tert-butyl (3RS,4RS)-4-[4-(3-benzyloxy-propoxy)-phenyl]-3-(3,4-dichloro-benzyloxy)-piperidine-1-carboxylate, which was used in the following step without further purification and characterization;

(tt) with 2,5-dichloro-benzyl chloride to give tert-butyl (3RS,4RS)-4-[4-(3-benzyloxy-propoxy)-phenyl]-3-(2,5-dichloro-benzyloxy)-piperidine-1-carboxylate, which was used in the following step without further purification and characterization;

(uu) with benzyl chloride to give tert-butyl (3RS,4RS)-3-benzyloxy-4-[4-(3-benzyloxy-propoxy)-phenyl]-piperidine-1-carboxylate, which was used in the following step without further purification and characterization,

(vv) with 2,5-dimethyl-benzyl chloride to give tert-butyl (3RS,4RS)-4-[4-(3-benzyloxy-propoxy)-phenyl]-3-(2,5-dimethyl-benzyloxy)-piperidine-1-carboxylate, which was used in the following step without further purification and characterization;

(ww) with 4-ethyl-benzyl chloride to give tert-butyl (3RS,4RS)-4-[4-(3-benzyloxy-propoxy)-phenyl]-3-(4-ethyl-benzyloxy)-piperidine-1-carboxylate, which was used in the following step without further purification and characterization;

(xx) with a mixture of 3- and 4-vinyl-benzyl chloride to give a mixture of tert-butyl (3RS,4RS)-4-[4-(3-benzyloxy-propoxy)-phenyl]-3-(3- and 4-vinyl-benzyloxy)-piperidine-1-carboxylate, which was used in the following step without further purification and characterization;

(yy) with 6-chloromethyl-2,3-dihydro-benzo[1,4]dioxin to give tert-butyl (3RS,4RS)-4-[4-(3-benzyloxy-propoxy)-phenyl]-3-(2,3-dihydro-benzo[1,4]dioxin-6-ylmethoxy)-piperidine-1-carboxylate, which was obtained as a colorless resin; MS: 607 (M+NH₄)⁺;

(zz) with 6-chloromethyl-1,2,3,4-tetrahydro-naphthalene to give tert-butyl (3RS,4RS)-4-[4-(3-benzyloxy-propoxy)-phenyl]-3-(5,6,7,8-tetrahydro-naphthalen-2-ylmethoxy)-piperidine-1-carboxylate, which was obtained as a colorless resin; MS: 603 (M+NH₄)⁺;

(aaa) with 3-chloromethyl-1-(2-trimethylsilanyl-ethoxymethoxy)-naphthalene [Example 5 (c)] to give tert-butyl (3RS,4RS)-4-[4-(3-benzyloxy-propoxy)-phenyl]-3-[4-(2-trimethylsilanyl-ethoxymethoxy)-naphthalen-2-ylmethoxy]-piperidine-1-carbxylate, which was obtained as a colorless resin; MS: 745 (M+NH₄)⁺.

(bbb) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 2-(4-chloro-butoxy)-tetrahydro-2H-pyran there was obtained a mixture of tert-butyl (3RS,4RS)- and (3SR,4SR)-3-hydroxy-4-[4-[4-[(RS)-tetrahydropyran-2-yloxy]-butoxy]-phenyl]-piperidine-1-carboxylate, alkylation of which with 2-bromomethyl-naphthalene analogously to Example 1 (g) gave a mixture of (3RS,4RS)- and (3SR,4SR)-3-(naphthalen-2-ylmethoxy)-4-[4-[4-[(RS)-tetrahydro-pyran-2-yloxy]-butoxy]-phenyl]-piperidine-1-carboxylate. Cleavage of the THP group with hydrogen chloride in methanol analogously to Example 53 (c) yielded tert-butyl (3RS,4RS)-4-[4-(4-hydroxy-butoxy)-phenyl]-3-(naphthalen-2-ylmethoxy)-piperidine1-carboxylate, alkylation of which with benzyl bromide analogously to Example 1 (g) gave tert-butyl (3RS,4RS)-4-[4-(4-benzyloxy-butoxy)-phenyl]-3-(naphthalen-2-ylmethoxy)-piperidine-1-carboxylate as a colorless oil; MS: 613 (M+NH₄)⁺.

(ccc) In an analogous manner to that described in Example 22 (d), from tert-butyl (3RS,4RS,5SR)-4-(4-bromo-phenyl)-5-methoxy-methyl-3-naphthalen-2-yl-methoxy-piperidine-1-carboxylate [Example 68 (l)] by a palladium-catalyzed carbonylation with carbon monoxide in methanol there was obtained tert-butyl (3SR,4RS,5RS)-4-(4-methoxycarbonyl-phenyl)-3-methoxymethyl-5-(naphthalen-2-ylmethoxy)-piperidine-1-carboxylate, reduction of which with lithium borhydride analogously to Example 22 (e) yielded tert-butyl (3SR,4RS,5RS)-4-(4-hydroxymethyl-phenyl)-3-methoxymethyl-5-(naphthalen-2-yl-methoxy)-piperidine-1-carboxylate. Subsequent acylation with 2-chlorobenzoyl chloride analogously to Example 22 (k) gave tert-butyl (3SR,4RS,5RS)-4-[4-(2-chloro-benzoyl-oxymethyl)-phenyl]-3-methoxymethyl-5-(naphthalen-2-ylmethoxy)-piperidine-]-carboxylate as a colorless foam; MS: 630 (M+H)⁺.

(ddd) In an analogous manner to that described in Example 1 (g), by alkylating tert-butyl 3SR,4RS,5RS)-4-(4-hydroxymethyl-phenyl)-3-methoxymethyl-5-(naphthalen-2-yl-methoxy)-piperidine-1-carboxylate with benzyl bromide there was obtained tert-butyl (3SR,4RS,5RS)-4-(4-benzyloxymethyl-phenyl)-3-methoxymethyl-5-(naphthalen-2-ylmethoxy)-piperidine-1-carboxylate as a colorless oil; MS: 582 (M+H)⁺.

(eee) In an analogous manner to that described in Example 12 (c)-(d), cleavage of the N-methyl group from (1RS,2RS,3RS,5SR)-3-(4-bromo-phenyl)-8-methyl-8-aza-bicyclo[3.2.1]octan-2-ol, obtained analogously to the 4-chlorophenyl derivative [J. Org. Chem. 35, 802 (1970)], was effected by firstly synthesizing (1RS,2RS,3RS,5SR)-3-(4-bromo-phenyl)-2-(2,2,2-trichloro-ethoxycarbonyloxy)-8-aza-bicyclo[3.2.1]octane-8-carboxylate by reaction with 2,2,2-trichloroethyl chloroformate and subsequently reacting with zinc in glacial acetic acid to give (1RS,2RS,3RS,5SR)-3-(4-bromo-phenyl)-8-aza-bicyclo[3.2.1]octan-2-ol. Subsequent introduction of the BOC group analogously to Example 1 (f) gave tert-butyl (1RS,2RS,3RS,5SR)-3-(4-bromo-phenyl)-2-hydroxy-8-aza-bicyclo[3.2.1]octane-8-carboxylate, palladium-catalyzed carbonylation of which with carbon monoxide in methanol analogously to Example 22 (d) yielded tert-butyl (1RS,2RS,3RS,5SR)-2-hydroxy-3-(4-methoxycarbonyl-phenyl)-8-aza-bicyclo[3.2.1]octane-8-carboxylate. Subsequent reduction with lithium borohydride analogously to Example 22 (e) gave tert-butyl (1RS,2RS,3RS,5SR)-2-hydroxymethyl-phenyl)-8-aza-bicyclo[3.2.1]octane-8-carboxylate, reaction of which with trityl chloride analogously to Example 22 (h) gave tert-butyl (1RS,2RS,3RS,5SR)-2-hydroxy-3-(4-trityloxymethyl)-phenyl)-8-aza-bicyclo[3.2.1]octane-8-carboxylate. Further reaction with 2-bromomethyl-naphthalene analogously to Example 1 (9) gave tert-butyl (1RS,2RS,3RS,5SR)-2-(naphthalen-2-ylmethoxy)-3-(4-trityloxymethyl-phenyl)-8-aza-bicyclo[3.2.1]octane-8-carboxylate. In an analogous manner to that described in Example 86 (u) (b), by cleavage of the trityl group with a mixture of trifluoroacetic acid and trifluoroacetic anhydride there was obtained tert-butyl (1RS,2RS,3RS,5SR)-3-(4-hydroxymethyl-phenyl)-2-(naphthalen-2-ylmethoxy)-8-aza-bicyclo[3.2.1]octane-8-carboxylate, alkylation of which with benzyl 3-bromopropyl ether analogously to Example 44 (e) gave tert-butyl (1RS,2RS,3RS,5SR)-3-[4-(2-benzyloxy-propoxy-methyl)-phenyl]-2-(naphthalen-2-ylmethoxy)-8-aza-bicyclo-[3.2.1]octane-8-carboxylate as a colorless oil.

### Example 87

The following compounds were obtained in an analogous manner to that described in Example 73 (d) by cleavage of the BOC group using anhydrous zinc bromide:

1)—From tert-butyl (3RS,4RS)-3-(naphthalen-2-carbonyloxy)-4-[4-(2-phenoxy-ethoxy)-phenyl]-piperidine-1-carboxylate, (3RS,4RS)-4-[4-(2-phenoxy-ethoxy)-phenyl]-piperidin-3-yl naphthalene-2-carboxylate as a colorless solid; MS: 468 (M+H)⁺;

2)—from a mixture of tert-butyl (3RS,4RS)-4-(4-fluoro-phenyl)-3-[(RS)- and (SR)-2-hydroxy-1-naphthalen-2-yl-ethoxy]-piperidine-1-carboxylate, a mixture of (RS)- and (SR)-2-[(3RS,4RS)-4-(4-fluoro-phenyl)-piperidine-3-yloxy]-2-naphthalen-2-yl-ethanol as a colorless, amorphous solid; MS: 366 (M+H)⁺.

The BOC derivatives used as the starting materials were prepared as follows:

(a) 250 mg of 4-dimethylamino-pyridine and 2.5 ml of triethylamine were added to a solution of 5.50 g (13.3 mmol) of tert-butyl (3RS,4RS)-3-hydroxy-4-[4-(2-phenoxy-ethoxy)-phenyl]-piperidine-1-carboxylate (Example 47.6) in 50 ml of methylene chloride and 2.77 g (14.5 mmol) of solid 2-naphthoyl chloride were subsequently added while cooling with ice. Thereupon, the reaction mixture was stirred at room temperature for 20 hours, partitioned between water and methylene chloride, the combined methylene chloride phases were dried over magnesium sulfate and concentrated, and the thus-obtained residue was chromatographed on silica gel with methylene chloride/ether (95:5). There were thus obtained 7.3 g (97% of theory) of tert-butyl (3RS,4RS)-3-(naphthalen-2-carbonyloxy)-4-[4-(2-phenoxy-ethoxy)-phenyl]-piperidine-1-carboxylate in the form of an amorphous, colorless solid; MS: 568 (M+H)⁺.

(b) Under argon and with the exclusion of moisture, 255 mg (0.57 mmol) of tert-butyl (3RS,4RS)-4-(4-fluoro-phenyl)-3-(1-naphthalen-2-yl-vinyloxy)-piperidine-1-carboxylate [Example 73 (b)] were dissolved in 3 ml of tetrahydrofuran, treated at 0° C. with 50 mg of triethylamine followed by 0.11 ml (about 1.1 mmol) of borane-dimethyl sulfide complex and stirred at room temperature for 30 minutes. Thereupon, again while cooling with ice, 1.5 ml of 50% KOH solution in water followed by 1.5 ml of 30% hydrogen peroxide solution in water were added and the reaction mixture was heated under reflux for 1.5 hours. Now, the reaction solution was partitioned between water and methylene chloride, the combined methylene chloride phases were dried over magnesium sulfate, concentrated and the thus-obtained residue was chromatographed on silica gel with hexane/ethyl acetate (1:1). There were thus obtained 30 mg (11% of theory) of a mixture of tert-butyl (3RS,4RS)-4-(4-fluoro-phenyl)-3-[(RS)- and (SR)-2-hydroxy-1-naphthalen-2-yl-ethoxy]-piperidine-1-carboxylate in the form of an amorphous, colorless solid; MS: 466 (M+H)⁺.

### Example 88

(a) 25.23 g (91 mmol) of tert-butyl 4-(4-fluoro-phenyl)-1,2,3,6-tetrahydro-pyridine-1-carboxylate [prepared from 4-(4-fluorophenyl)-1,2,3,6-tetrahydro-pyridine and di-tert-butyl dicarbonate in analogy to Example 1 (f)] were suspended in 200 ml of 1,2-dimethoxyethane, 5.1 g (135 mmol) of sodium borohydride were added thereto at 20° C. and subsequently a solution of 22.85 ml (182 mmol) of boron trifluoride ethyl etherate in 35 ml of 1,2-dimethoxyethane was added dropwise during 45 minutes while cooling occasionally at 20° C. After stirring at room temperature for 2½ hours a solution of 82 g (1.26 mmol) of potassium hydroxide (86%) in 430 ml of distilled water was added dropwise at room temperature during 1 hour while stirring intensively. Thereupon, 69.3 ml (0.68 mmol) of hydrogen peroxide (30%) were added dropwise at room temperature within 30 minutes and then the mixture was stirred at reflux for 3 hours. After cooling to room temperature the mixture was poured on to ice-water, the product was extracted 3 times with 200 ml of ethyl acetate each time, the organic phases were washed twice with 300 ml of distilled water each time, then dried over magnesium sulfate, filtered and concentrated in a water-jet vacuum. After drying in a high vacuum at room temperature for 90 minutes there were thus obtained 24.4 g (91% of theory) of tert-butyl (3RS,4RS)-4-(4-fluoro-phenyl)-3-hydroxy-piperidine-1-carboxylate in the form of colorless crystals; MS: 296 (M+H)⁺.

(b) 39.2 g (0.2 mol) of 3-methylbenzophenone and 38.4 g (0.24 mol) of bromine were stirred at reflux in 1 l of carbon tetrachloride for 8 hours. After distillation of the solvent in a water-jet vacuum the crude product obtained was chromatographed on 500 g of silica gel with hexane and methylene chloride. The thus-purified product was recrystallized from n-hexane. There were thus obtained 21.76 g (40% of theory) of (3-bromomethyl-phenyl)-phenyl-methanone in the form of colorless crystals; MS: 274, 276 (M)⁺.

(c) 0.29 g (1 mmol) of tert-butyl (3RS,4RS)-4-(4-fluoro-phenyl)-3-hydroxy-piperidine-1-carboxylate and 0.30 g (1.1 mmol) of (3-bromomethyl-phenyl)-phenyl-methanone were dissolved in 10 ml of dimethylformamide under argon at room temperature, then treated with 0.056 g (1.3 mmol) of sodium hydride dispersion (55% in mineral oil) with the addition of 0.25 g (1.5 mmol) of potassium iodide and stirred at room temperature for 18 hours. The reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.42 g (86% of theory) of tert-butyl (3RS,4RS)-3-(3-benzoyl-benzyloxy)-4-(4-fluoro-phenyl)-piperidine-1-carboxy late as a colorless oil; MS: 490 (M+H)⁺.

(d) In an analogous manner to that described in Example 22 (l), from tert-butyl (3RS,4RS)-3-(3-benzoyl-benzyloxy)-4-(4-fluoro-phenyl)-piperidine-1-carboxylate by cleavage of the BOC group with hydrogen chloride in methanol there was obtained [3-[(3RS,4RS)-4-(4-fluoro-phenyl)-piperidin-3-yloxymethyl]-phenyl]-phenyl-methanone hydrochloride (1:1) in the form of colorless crystals; MS: 390 (M+H)⁺.

### Example 89

The following compounds were obtained in an analogous manner to that described in Example 73 (d) by cleavage of the BOC group using anhydrous zinc bromide:

1)—From tert-butyl (E)-(3RS,4RS)-4-(4-fluoro-phenyl)-3-(3-phenyl-allyloxy)-piperidine-1-carboxylate, (E)-(3RS,4RS)-4-(4-fluoro-phenyl)-3-(3-phenyl-allyloxy)-piperidine as a pale yellow oil; MS: 312 (M+H)⁺;

2)—from tert-butyl (3R,4s,5S)-4-[4-(3-benzyloxy-propoxy)-phenyl]-3,5-bis-(4,5-dimethoxy-pyrimidin-2-yloxymethyl)-piperidine-1-carboxylate, 2-[(3R,4s,5S)-4-[4-(3-benzyloxy-propoxy)-phenyl]-5-(4,5-dimethoxy-pyrimidin-2-yloxymethyl)-piperidine-3-ylmethoxy}-4,5-dimethoxy-pyrimidine as a pale yellow resin; MS: 662 (M+H)⁺;

3)—from tert-butyl (3R,4s,5S)-4-[4-(3-benzyloxy-propoxy)-phenyl]-3,5-bis-(4-methoxy-phenoxymethyl)-piperidine-1-carboxylate, (3R,4S,5S)-4-[4-(3-benzyloxy-propoxy)-phenyl]-3,5-bis-(4-methoxy-phenoxymethyl)-piperidine as a pale yellow oil; MS: 598 (M+H)⁺;

4)—from tert-butyl (3R,4s,5S)-4-[4-(3-benzyloxy-propoxy)-phenyl]-3,5-bis-p-tolylsulfanylmethyl-piperidine-1-carboxylate, (3R,4s,5S)-4-[4-(3-benzyloxy-propoxy)-phenyl]-3,5-bis-p-tolylsulfanylmethyl-piperidine as a pale yellow oil; MS: 598 (M+H)⁺.

5)—from (3RS,4RS)-4-[4-[3-(2-methoxy-benzyloxy)-propoxy]-phenyl]-3-[7-[2-(4-methyl-piperazin-1-yl)-ethoxy]-naphthalen-2-ylmethoxy]-piperidin-1-carboxylic acid tert-butylester, 1-[2-[7-[(3RS,4RS)-4-[4-[3-(2-methoxy-benzyloxy)-propoxy]-piperidin-3-yloxymethyl)-naphthalen-2-yloxy]-ethyl)-4-methyl-piperazine as a amorphous, colorless solid; MS: 654 (M+H)⁺.

The BOC derivatives used as the starting materials were prepared as follows:

(a) 1.48 g (5 mmol) of tert-butyl (3RS,4RS)-4-(4-fluoro-phenyl)-3-hydroxy-piperidine-1-carboxylate [Example 88 (a)] and 1.08 g (5.5 mmol) of 3-bromo-1-phenyl-propene were dissolved in 10 ml of dimethylformamide under argon at room temperature, then treated with 0.284 g (6.5 mmol) of sodium hydride dispersion (55% in mineral oil) with the addition of 1.25 g (7.5 mmol) of potassium iodide and stirred at room temperature for 18 hours. The reaction mixture was poured on to ice-water, the product was extracted 3 times with methylene chloride, the organic phases were washed twice with distilled water, dried over magnesium sulfate, filtered and concentrated in a water-jet vacuum. The thus-obtained crude product was chromatographed on silica gel with methylene chloride and methanol. There were thus obtained 1.01 g (49% of theory) of tert-butyl (E)-(3RS,4RS)-4-(4-fluoro-phenyl)-3-(3-phenyl-allyloxy)-piperidine-1-carboxylate as a colorless oil; MS: 412 (M+H)⁺.

(b) 0.49 g (1 mmol) of tert-butyl (3R,4s,5S)-4-[4-(3-benzyloxy-propoxy)-phenyl]-3,5-bis-hydroxymethyl-piperidine-1-carboxylate [Example 101 (f)] and 0.45 g (2 mmol) of 4,5-dimethoxy-2-methylsulfonyl-pyrimidine [prepared from 4,5-dimethoxy-2-methylsulfanyl-pyrimidine by oxidation with m-chloroperbenzoic acid in an analogous manner to that described in Example 129 (c)] were placed in 5 ml of dimethylformamide under argon at 5° C., treated while stirring with 0.10 g (2.2 mmol) of sodium hydride dispersion (55% in mineral oil) and stirred at room temperature for 2 hours. The reaction mixture was thereupon poured on to ice-water, the product was extracted 3 times with methylene chloride, the organic phases were washed twice with distilled water, dried over magnesium sulfate, filtered and concentrated in a water-jet vacuum. The thus-obtained crude product was chromatographed on silica gel with methylene chloride and methanol. There was thus obtained 0.47 g (61% of theory) of tert-butyl (3R,4s,5S)-4-[4-(3-benzyloxy-propoxy)-phenyl]-3,5-bis-(4,5-dimethoxy-pyrimidin-2-yloxymethyl)-piperidine-1-carboxylate as a colorless oil; MS: 762 (M+H)⁺.

(c) (α) 2.91 g (6 mmol) of tert-butyl (3R,4s,5S)-4-[4-(3-benzyloxy-propoxy)-phenyl]-3,5-bis-hydroxymethyl-piperidine-1-carboxylate [Example 101 (f)] and 1.93 ml (24 mmol) of pyridine were placed in 30 ml of acetonitrile under argon at 5° C., 8.00 g (18 mmol) of triphenylphosphine dibromide were introduced portionwise while stirring and the mixture was thereafter stirred at room temperature. After 90 minutes the reaction mixture was poured on to ice-water, the product was then extracted 3 times with ethyl acetate, the organic phases were washed twice with distilled water, dried over magnesium sulfate, filtered and concentrated in a water-jet vacuum. The thus-obtained crude product was chromatographed on silica gel with n-hexane and ethyl acetate. There were thus-obtained 2.81 g (77% of theory) of tert-butyl (3R,4s,5S)-4-[4-(3-benzyloxy-propoxy)-phenyl]-3,5-bis-bromomethyl-piperidine-1-carboxylate as a colorless oil; MS: 610 (M+H)⁺.

(c) (β) 0.30 g (0.5 mmol) of tert-butyl (3R,4s,5S)-4-[4-(3-benzyloxy-propoxy)-phenyl]-3,5-bis-bromomethyl-piperidine-1-carboxylate and 0.19 g (1.5 mmol) of hydroquinone monomethyl ether were stirred at reflux under argon for 18 hours with the addition of 0.69 g (5 mmol) of anhydrous potassium carbonate in 15 ml of acetonitrile. After cooling to room temperature the reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.15 g (43% of theory) of tert-butyl (3R,4s,5S)-4-[4-(3-benzyloxy-propoxy)-phenyl]-3,5-bis-(4-methoxy-phenoxymethyl)-piperidine-1-carboxylate as pale yellow crystals; MS: 698 (M+H)⁺.

(d) In an analogous manner to that described in Example 89 (c) (β), from tert-butyl (3R,4s,5S)-4-[4-(3-benzyloxy-propoxy)-phenyl]-3,5-bis-bromomethyl-piperidine-1-carboxylate and 4-methyl-thiophenol there was obtained tert-butyl (3R,4s,5S)-4-[4-(3-benzyloxy-propoxy)-phenyl]-3,5-bis-p-tolylsulfanylmethyl-piperidine-1-carboxylate in the form of a colorless oil; MS: 698 (M+H)⁺.

(e) in an analogeous manner as described in Example 95 (a) there was obtained from (3RS,4RS)-3-hydroxy-4-[4-[3-(2-methoxy-benzyloxy)-propoxy]-phenyl]-piperidin-1-carboxylic acid tert-butylester [Example 120 (g) (a)] and 2-chloromethyl-7-(2-trimethylsilanyl-ethoxymethoxy)-naphthalene [Example 6 (u)] (3RS,4RS)-4-[4-[3-(2-methoxy-benzyloxy)-propoxy]-phenyl]-3-[7-(2-trimethyl silanyl-ethoxymethoxy)-naphthalen-2-ylmethoxy]-piperidin-1-carboxylic acid tert-butylester as a pale yellow oil; MS: 758 (M+H)⁺. Then, (3RS,4RS)-4-[4-[3-(2-methoxy-benzyloxy)-propoxy]-phenyl]-3-[7-(2-trimethyl silanyl-ethoxymethoxy)-naphthalen-2-ylmethoxy]-piperidin-1-carboxylic acid tert-butylester was reacted in analogy to Example 95 (b) by cleaving of the SEM protecting group to afford (3RS,4RS)-4-[4-[3-(2-methoxy-benzyloxy)-propoxy]-phenyl]-3-(7-hydroxy-naphthalen-2-ylmethoxy)-piperidin-1-carboxylic acid tert-butylester [yellow oil; MS: 628 (M+H)+] which, on alkylation with 1-(2-chloro-ethyl)-4-methyl-piperazine hydrochloride (1:2) [Chim. Ther. 4, 283 (1969)] in analogy to Example 90 (n) gave (3RS,4RS)-4-[4-[3-(2-methoxy-benzyloxy)-propoxy]-phenyl]-3-[7-[2-(4-methyl-piperazin-1-yl)-ethoxy]-naphthalen-2-ylmethoxy]-piperidin-1-carboxylic acid tert-butylester as a light brown oil; MS: 754 (M+H)⁺.

### Example 90

The following compounds were obtained in an analogous manner to that described in Example 22 (l) by cleavage of the BOC group using hydrogen chloride in methanol:

1)—From a mixture of tert-butyl (3RS,4SR)-4-(4-fluoro-phenyl)-3-[(RS)- and -[(SR)-1-[4-(2-morpholin-4-yl-ethoxy)-benzoyloxy]-2-naphthalen-2-yl-ethyl]-piperidine-1-carboxylate, a mixture of (RS)- and (SR)-1-[(3RS,4SR)-4-(4-fluoro-phenyl)-piperidin-3-yl]-2-naphthalen-2-yl-ethyl 4-(2-morpholin-4-ylethoxy)-benzoate as a colorless oil; MS: 583 (M+H)⁺;

2)—from tert-butyl (3RS,4SR)-4-(4-fluoro-phenyl)-3-(naphthalen-2-yl-acetyl)-piperidine-1-carboxylate, 1-[(3RS,4SR)-4-(4-fluoro-phenyl)-piperidin-3-yl]-2-naphthalen-2-yl-ethanone hydrochloride (1:1) in the form of colorless crystals; MS: 348 (M+H)⁺;

3)—from a mixture of tert-butyl (E)- and (Z)-(3RS,4SR)-3-(1-carboxymethoxyimino-2-naphthalen-2-yl-ethyl)-4-(4-fluoro-phenyl)-piperidine-1-carboxylate, a 3:1 mixture of methyl (E)- and (Z)-[1-[(3RS,4SR)-4-(4-fluoro-phenyl)-piperidine-3-yl]-2-naphthalen-2-yl-ethylidene-aminooxy]-acetate as a yellow oil; MS: 435 (M+H)⁺;

4)—from tert-butyl (3RS,4RS)-3-[2-(3H-benzoimidazol-5-yloxy)-ethoxy]-4-[4-(3-benzyloxy-propoxy)-phenyl]-piperidine-1-carboxylate, 6-[2-[(3RS,4RS)-4-[4-(3-benzyloxy-propoxy)-phenyl]-piperidin-3-yloxy}-ethoxy]-1H-benzoimidazole as a yellow oil; MS: 502 (M+H)⁺;

5)—from tert-butyl 3RS,4RS)-4-[4-(3-benzyloxy-propoxy)-phenyl]-3-[2-(2-oxo-2,3-dihydro-1H-benzoimidazol-5-yloxy)-ethoxy]-piperidine-1-carboxylate, 5-[2-[(3RS,4RS)-4-[4-(3-benzyloxy-propoxy)-phenyl]-piperidin-3-yloxy]-ethoxy]-1,3-dihydro-benzoimidazol-2-one in the form of yellow crystals; MS: 518 (M+H)⁺;

6)—from tert-butyl (3RS,4RS)-4-[4-(3-benzyloxy-propoxy)-phenyl]-3-[2-(3,4-dinitro-phenoxy)-ethoxy]-piperidine-1-carboxylate [Example 94 (d)], (3RS,4RS)-4-[4-(3-benzyloxy-propoxy)-phenyl]-3-[2-(3,4-dinitro-phenoxy)-ethoxy]-piperidine as an amorphous, yellow foam; MS: 552 (M+H)⁺;

7)—from tert-butyl (3RS,4RS)-4-[4-(3-benzyloxy-propoxy)-phenyl]-3-[7-(2-morpholin-4-yl-ethoxy)-naphthalen-2-ylmethoxy]-piperidine-1-carboxylate, 4-[2-[7-[(3RS,4RS)-4-[4-(3-benzyloxy-propoxy)-phenyl]-piperidin-3-yloxymethyl]-naphthalen-2-yloxy]-ethyl]-morpholine hydrochloride (1:2) in the form of beige crystals; MS: 611 (M+H)⁺;

8)—from a mixture of tert-butyl (3RS,4RS)- and (3SR,4SR)-4-[4-(3-benzyloxy-propoxy)-phenyl]-3-[7-[(RS)-2,2-dimethyl-[1,3]dioxolan-4-ylmethoxy]-naphthalen-2-ylmethoxy]-piperidine-1-carboxylate, a mixture of (RS)- and (SR)-3-[7-[(3RS,4RS)-4-[4-(3-benzyloxy-propoxy)-phenyl]-piperidin-3-yloxymethyl]-naphthalen-2-yloxy]-propane-1,2-diol as a colorless oil; MS: 572 (M+H)⁺;

9)—from tert-butyl (3RS,4RS)-4-[4-(3-benzyloxy-propoxy)-phenyl]-3-[6-(2-trimethylsilanyl-ethoxymethoxy)-naphthalen-2-ylmethoxy]-piperidine-1-carboxylate [Example 97 (a)], 6-[(3RS,4RS)-4-[4-(3-benzyloxy-propoxy)-phenyl]-piperidin-3-yloxymethyl]-naphthalen-2-ol hydrochloride (1:1) in the form of colorless crystals; MS: 498 (M+H)⁺;

10)—from a mixture of tert-butyl[3RS,4RS]- and [3SR,4SR]-4-[4-(3-benzyloxy-propoxy)-phenyl]-3-[6-[(RS)-2,2-dimethyl-[1,3]dioxolan-4-ylmethoxy)-naphthalen-2-ylmethoxy]-piperidine-1-carboxylate with simultaneous cleavage of the dioxolane protecting group, a mixture of (RS)- and (SR)-3-[6-[(3RS,4RS)-4-[4-(3-benzyloxy-propoxy)-phenyl]-piperidin-3-yloxymethyl]-naphthalen-2-yloxy]-propane-1,2-diol hydrochloride (1:1) in the form of pale brown crystals; MS: 572 (M+H)⁺;

11)—from a mixture of tert-butyl (3RS,4RS)- and (3SR,4SR)-4-[4-(3-benzyloxy-propoxy)-phenyl]-3-[6-[2-[(RS)-2,2-dimethyl-[1,3]dioxolan-4-ylmethoxy]-ethoxy]-naphthalen-2-ylmethoxy]-piperidine-1-carboxylate with simultaneous cleavage of the dioxolane protecting group, a mixture of [RS]- and [SR]-3-[2-[6-[(3RS,4RS)-4-[4-(3-benzyloxy-propoxy)-phenyl]-piperidin-3-yloxymethyl]-naphthalen-2-yloxy]-ethoxy]-propane-1,2-diol hydrochloride (1:1) in the form of colorless crystals; MS: 616 (M+H)⁺;

12)—from a mixture of tert-butyl (3RS,4SR)-4-[4-(3-benzyloxy-propoxy)-phenyl]-3-[(RS)- and -[(SR) 1-hydroxy-2-naphthalen-2-yl-ethyl]-piperidine-1-carboxylate [Example 100 (b)], a mixture of (RS)- and (SR)-1-[(3RS,4SR)-4-[4-(3-benzyloxy-propoxy)-phenyl]-piperidin-3-yl]-2-naphthalen-2-yl-ethanol hydrochloride (1:1) in the form of beige crystals; MS: 496 (M+H)⁺;

13) from tert-butyl (3RS,4RS)-4-[4-(3-benzyloxy-propoxy)-phenyl]-3-[7-[2-(4-methyl-piperazin-1-yl)-ethoxy]-naphthalen-2-ylmethoxy]-piperidine-1-carboxylate, 1-[2-[7-[(3RS,4RS)-4-[4-(3-benzyloxy-propoxy)-phenyl]-piperidin-3-yloxymethyl]-naphthalen-2-yloxy]-ethyl]-4-methyl-piperazine hydrochloride (1:3) in the form of colorless crystals; MS: 624 (M+H)⁺;

14) from a mixture of tert-butyl (E)- and (Z)-(3RS,4SR)-4-[4-(3-benzyloxy-propoxy)-phenyl]-3-(1-methoxycarbonyl-methoxyimino-2-naphthalen-2-yl-ethyl)-piperidine-1-carboxylate, a mixture of methyl (E)- and (Z)-(1-[(3RS,4SR)-4-[4-(3-benzyloxy-propoxy)-phenyl]-piperidin-3-yl]-2-naphthalen-2-yl-ethylideneaminooxy)-acetate as a pale yellow oil; MS: 581 (M+H)⁺;

15) from tert-butyl (3R,4s,5S)-4-[4-(3-benzyloxy-propoxy)-phenyl]-3,5-bis-(2-morpholin-4-yl-ethoxymethyl)-piperidine-1-carboxylate [Example 101 (g)], (3R,4s,5S)-4-[4-(3-benzyloxy-propoxy)-phenyl-3,5-bis-(2-morpholin-4-yl-ethoxymethyl)-piperidine as a yellow oil; MS: 612 (M+H)⁺.

The BOC derivatives used as the starting materials were prepared as follows:

(a) 0.45 g (1 mmol) of a mixture of tert-butyl (3RS,4SR)-4-(4-fluoro-phenyl)-3-[(RS)- and -[(SR)-1-hydroxy-2-naphthalen-2-yl-ethyl]-piperidine-1-carboxylate [Example 74 (h)] and 0.28 g (1.1 mmol) of 4-(2-morpholin-4-yl-ethoxy)-benzoic acid (prepared by alkylating methyl 4-hydroxybenzoate with 4-(2-chloroethyl)-morpholine in dimethylformamide in the presence of potassium carbonate at 100° C. and subsequently saponifying with base were dissolved in 15 ml of methylene chloride under argon, 0.23 g (1.2 mmol) of N-ethyl-N′-(3-dimethylaminopropyl)-carbodiimide hydrochloride and 0.04 g (0.33 mmol) of 4-dimethylamino-pyridine were then added and the mixture was stirred at room temperature for 70 hours. After distillation of the solvent in a water-jet vacuum the crude product was chromatographed on silica gel with n-hexane, methylene chloride and methanol. There was thus obtained 0.54 g (79% of theory) of a mixture of tert-butyl (3RS,4SR)-4-(4-fluoro-phenyl)-3-[(RS)- and -[(SR)-1-[4-(2-morpholin-4-yl-ethoxy)-benzoyloxy]-2-naphthalen-2-yl-ethyl]-piperidine-1-carboxylate as a pale yellow oil; MS: 683 (M+H)⁺.

(b) In an analogous manner to that described in Example 74 (f), from a mixture of tert-butyl (3RS,4SR)-4-(4-fluoro-phenyl)-3-[(RS)- and -[(SR)-1-hydroxy-2-naphthalen-2-yl-ethyl]-piperidine-1-carboxylate [Example 74 (h)] by oxidation with dimethyl sulfoxide/oxalyl chloride in methylene chloride there was obtained tert-butyl (3RS,4SR)-4-(4-fluoro-phenyl)-3-(naphthalen-2-yl-acetyl)-piperidine-1-carboxylate as a yellow oil; MS: 447 (M)⁺.

(c) 0.22 g (0.5 mmol) of tert-butyl (3RS,4SR)-4-(4-fluoro-phenyl)-3-(naphthalen-2-yl-acetyl)-piperidine-1-carboxylate and 0.11 g (1 mmol) of aminooxy-acetic acid hydrochloride (1:0.5) [Organic Synthesis Collect. Vol. III, 172 (1955)] were stirred at 60° C. in 2 ml of pyridine for 18 hours under argon. The reaction mixture was thereupon poured on to ice-water, the product was extracted 3 times with ethyl acetate, the organic phases were washed twice with distilled water, dried over magnesium sulfate, filtered and concentrated in a water-jet vacuum. The thus-obtained crude product was chromatographed on silica gel with methylene chloride and methanol. There was thus obtained 0.24 g (92% of theory) of a mixture of tert-butyl (E)- and (Z)-(3RS,4SR)-3-(1-carboxymethoxyimino-2-naphthalen-2-yl-ethyl)-4-(4-fluor o-phenyl)-piperidine-1-carboxylate as a pale yellow oil; MS: 521 (M+H)⁺.

(d) 0.55 g (0.93 mmol) of tert-butyl (3RS,4RS)-4-[4-(3-benzyloxy-propoxy)-phenyl]-3-[2-(3,4-diamino-phenoxy)-ethoxy]-piperidine-1-carboxylate (Example 94 (e)] was stirred at 50° C. in 5 ml of triethyl orthoformate for 1 hour under argon. The reaction mixture was poured on to ice-water, the product was extracted 3 times with methylene chloride, the organic phases were washed twice with distilled water, then dried over potassium carbonate, filtered and concentrated in a water-jet vacuum. The thus-obtained crude product was chromatographed on silica gel with methylene chloride and methanol. There was thus obtained 0.38 g (68% of theory) of tert-butyl 3-[2-(3H-benzoimidazol-5-yloxy)-ethoxy]-4-[(3RS,4RS)-4-(3-benzyloxy-propoxy)-phenyl]-piperidine-1-carboxylate as a pale brown oil; MS: 602 (M+H)⁺.

(e) 0.60 g (1.0 mmol) of tert-butyl (3RS,4RS)-4-[4-(3-benzyloxy-propoxy)-phenyl]-3-[2-(3,4-diamino-phenoxy)-ethoxy]-piperidine-1-carboxylate [Example 94 (e)] was dissolved in 5 ml of dimethylformamide under argon a room temperature and then 0.18 g (1.1 mmol) of 1,1′-carbonyldiimidazole was added. After 1 hour the reaction mixture was poured on to ice to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.46 g (74% of theory) of tert-butyl (3RS,4RS)-4-[4-(3-benzyloxy-propoxy)-phenyl]-3-[2-(2-oxo-2,3-dihydro-1H-benzo-imidazol-5-yloxy)-ethoxy]-piperidine-1-carboxylate as a yellow oil; MS: 618 (M+H)⁺.

(g) 0.30 g (0.5 mmol) of tert-butyl (3RS,4RS)-4-[4-(3-benzyloxy-propoxy)-phenyl]-3-(7-hydroxy-naphthalen-2-ylmethoxy)-piperidine-1-carboxylate [Example 95 (b)] and 0.13 g (0.7 mmol) of 4-(2-chloroethyl)-morpholine hydrochloride were stirred at 60° C. under argon for 18 hours in 15 ml of dimethylformamide with the addition of 0.69 g (5 mmol) of potassium carbonate (anhydrous). The reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.32 g (90% of theory) of tert-butyl (3RS,4RS)-4-[4-(3-benzyloxy-propoxy)-phenyl]-3-[7-(2-morpholin-4-yl-ethoxy)-naphthalen-2-ylmethoxy]-piperidine-1-carboxylate as a colorless oil; MS: 711 (M+H)⁺.

(h) 0.33 g (0.54 mmol) of tert-butyl (3RS,4RS)-4-[4-(3-benzyloxy-propoxy)-phenyl]-3-(7-hydroxy-naphthalen-2-ylmethoxy)-piperidine-1-carboxylate [Example 95 (b)] and 0.20 g (0.70 mmol) of D,L-α,β-isopropylideneglycerol γ-tosylate were stirred at reflux for 3 hours under argon in 15 ml of dimethyl-formamide with the addition of 0.69 g (5 mmol) of potassium carbonate (anhydrous). The reaction mixture was thereupon poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.32 g (83% of theory) of a mixture of tert-butyl (3RS,4RS)- and (3SR,4SR)-4-[4-(3-benzyloxy-propoxy)-phenyl]-3-[7-[(RS)-2,2-dimethyl-[1,3]dioxolan-4-ylmethoxy]-naphthalen-2-ylmethoxy]-piperidine-1-carboxylate as a colorless oil; MS: 712 (M+H)⁺.

(k) In an analogous manner to that described in Example 90 (h), from tert-butyl (3RS,4RS)-4-[4-(3-benzyloxy-propoxy)-phenyl]-3-(6-hydroxy-naphthalen-2-ylmethoxy)-piperidine-1-carboxylate [Example 97 (b)] there was obtained a mixture of tert-butyl [3RS,4RS]- and [3SR,4SR]-4-[4-(3-benzyloxy-propoxy)-phenyl]-3-[6-[(RS)-2,2-dimethyl-[1,3]dioxolan-4-ylmethoxy)-naphthalen-2-ylmethoxy)-piperidine-1-carboxylate in the form of colorless crystals; MS: 712 (M+H)⁺.

(l) In an analogous manner to that described in Example 90 (g), from tert-butyl (3RS,4RS)-4-[4-(3-benzyloxy-propoxy)-phenyl]-3-(6-hydroxy-naphthalen-2-ylmethoxy)-piperidine-1-carboxylate [Example 97 (b)] and (RS)-2-(2,2-dimethyl-[1,3]dioxolan-4-ylmethoxy)-ethyl toluene 4-sulfonate [Example 98 (a)] there was obtained a mixture of tert-butyl (3RS,4RS)- and (3SR,4SR)-4-[4-(3-benzyloxy-propoxy)-phenyl]-3-[6-[2-[(RS)-2,2-dimethyl-[1,3]dioxolan-4-ylmethoxy]-ethoxy]-naphthalen-2-ylmethoxy]-piperidine-1-carboxylate in the form of colorless crystals; MS: 773 (M+NH₄)⁺.

(n) 0.30 g (0.5 mmol) of tert-butyl (3RS,4RS)-4-[4-(3-benzyloxy-propoxy)-phenyl]-3-(7-hydroxy-naphthalen-2-yl-methoxy)-piperidine-1-carboxylate [Example 95 (b)] and 0.47 g (2 mmol) of 1-(2-chloro-ethyl)-4-methyl-piperazine hydrochloride (1:2) [Chim. Ther. 4, 283 (1969)] were dissolved in 5 ml of dimethylformamide under argon at room temperature and then stirred at 100° C. for 3 hours with the addition of 0.05 g (0.3 mmol) of potassium iodide and 0.22 g (5 mmol) of sodium hydride dispersion (55% in mineral oil). The reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.20 g (55% of theory) of tert-butyl (3RS,4RS)-4-[4-(3-benzyloxy-propoxy)-phenyl]-3-[7-[2-(4-methyl-piperazin-1-yl)-ethoxy]-naphthalen-2-ylmethoxy]-piperidine-1-carboxylate as a pale yellow oil; MS: 724 (M+H)⁺.

(o) In an analogous manner to that described in Example 102 (a), from tert-butyl (3RS,4SR)-4-[4-(3-benzyloxy-propoxy)-phenyl]-3-(naphthalen-2-yl-acetyl)-piperidine-1-carboxylate [Example 100 (c)] and methyl aminooxy-acetate hydrochloride [J. Med. Chem. 28, 1447 (1985)] there was obtained a mixture of tert-butyl (E)- und (Z)-(3RS,4SR)-4-[4-(3-benzyloxy-propoxy)-phenyl]-3-(1-methoxycarbonylmethoxyimino-2-naphthalen-2-yl-ethyl)-piperidine-1-carboxylate as a colorless oil; MS: 698 (M+NH₄)⁺.

### Example 91

(a) 0.90 g (2 mmol) of a mixture of tert-butyl (3RS,4SR)-4-(4-fluoro-phenyl)-3-[(RS)- and -[(SR)-1-hydroxy-2-naphthalen-2-yl-ethyl]-piperidine-1-carboxylate [Example 74 (h)] and 0.55 g (2.2 mmol) of 4-(2-morpholin-4-yl-ethoxy]-benzoic acid (prepared by alkylating methyl 4-hydroxybenzoate with 4-(2-chloroethyl)morpholine in dimethylformamide in the presence of potassium carbonate at 100° C. and subsequently saponifying with base) were dissolved in 30 ml of tetrahydrofuran under argon, then 0.44 ml (2.8 mmol of diethyl azodicarboxylate was added dropwise after the addition of 0.66 g (2.5 mmol) of triphenylphosphine and the mixture was subsequently stirred at room temperature for 18 hours. After distillation of the solvent in a water-jet vacuum the crude product was purified on silica gel with n-hexane and ethyl acetate. There was thus obtained 0.31 g (23% of theory) of tert-butyl (E)-(3RS,4SR)-4-(4-fluoro-phenyl)-3-(2-naphthalen-2-yl-vinyl)-piperidine-1-carboxylate as a pale yellow resin; MS: 432 (M+H)⁺.

(b) In an analogous manner to that described in Example 22 (l), by cleaving off the BOC group using hydrogen chloride in methanol from tert-butyl (E)-(3RS,4SR)-4-(4-fluoro-phenyl)-3-(2-naphthalen-2-yl-vinyl)-piperidine-1-carboxylate there was obtained (E)-(3RS,4SR)-4-(4-fluoro-phenyl)-3-(2-naphthalen-2-yl-vinyl)-piperidine as a pale yellow oil; MS: 331 (M)⁺.

(c) 0.060 g (0.18 mmol) of (E)-(3RS,4SR)-4-(4-fluoro-phenyl)-3-(2-naphthalen-2-yl-vinyl)-piperidine was hydrogenated under normal conditions in 3 ml of methanol with the addition of 30 mg of Pd—C (10%). After filtering off the catalyst the solvent was distilled off in a water-jet vacuum. There was thus obtained 0.055 g (92% of theory) of (3RS,4SR)-4-(4-fluoro-phenyl)-3-(2-naphthalen-2-yl-ethyl)-piperidine as a pale yellow oil; MS: 334 (M+H)⁺.

### Example 92

(a) 2.95 g (10 mmol) of tert-butyl (3RS,4RS)-4-(4-fluoro-phenyl)-3-hydroxy-piperidine-1-carboxylate [Example 88 (a)] and 3.82 g (11 mmol) of tert-butyl 4′-bromomethyl-biphenyl-2-carboxylate [J. Med. Chem. 34, 2525 (1991)] were dissolved in 100 ml of dimethylformamide under argon at room temperature, then firstly 2.49 g (15 mmol) of potassium iodide and thereafter 0.57 g (13 mmol) of sodium hydride dispersion (55% in mineral oil) were added. After stirring at room temperature for 4 hours the reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with n-hexane and ethyl acetate. There were thus obtained 5.08 g (90% of theory) of tert-butyl (3RS,4RS)-3-(2′-tert-butoxycarbonyl-biphenyl-4-ylmethoxy)-4-(4-fluoro-phenyl)-piperidine-1-carboxylate as an amorphous, colorless foam.

(b) 2.25 g (4 mmol) of tert-butyl (3RS,4RS)-3-(2′-tert-butoxycarbonyl-biphenyl-4-ylmethoxy)-4-(4-fluoro-phenyl)-piperidine-1-carboxylate were stirred at reflux for 18 hours in 50 ml of ethylene glycol monomethyl ether with the addition of 8 ml (36 mmol) of sodium hydroxide solution (14%). After distillation of the solvent in a water-jet vacuum the residue was dissolved in ice-water, then adjusted to pH 3 with 2 N hydrochloric acid and the product was extracted 3 times with methylene chloride, the organic phases were washed twice with distilled water, then dried over magnesium sulfate, filtered and concentrated in a water-jet vacuum. The thus-obtained product was chromatographed on silica gel with methylene chloride and methanol. There were thus obtained 1.66 g (82% of theory) of tert-butyl (3RS,4RS)-3-(2′-carboxy-biphenyl-4-ylmethoxy)-4-(4-fluoro-phenyl)-piperidine-1-carboxylate as an amorphous, colorless foam; MS: 504 (M-H)⁺.

(c) 0.51 g (1 mmol) of tert-butyl (3RS,4RS)-3-(2′-carboxy-biphenyl-4-ylmethoxy)-4-(4-fluoro-phenyl)-piperidine-1-carboxylate and 0.18 g (1 mmol) of 2-chloro-4,6-dimethoxy-1,3,5-triazine were dissolved in a mixture of 3.5 ml of dimethylformamide and 5 ml of acetonitrile under argon, 0.22 ml (2 mmol) of N-methylmorpholine was added dropwise thereto at 0° C. and the mixture was stirred at 0° C. for 2 hours. Now, a solution of 0.08 ml (1 mmol) of (RS)-3-amino-1,2-propanediol in 6 ml of acetonitrile was added dropwise and thereafter the mixture was stirred at room temperature for 18 hours. The reaction mixture was poured on to ice-water, the product was extracted 3 times with methylene chloride, the organic phases were washed twice with distilled water, then dried over magnesium sulfate, filtered and concentrated in water-jet vacuum. The thus-obtained crude product was chromatographed on silica gel with methylene chloride and methanol. There was thus obtained 0.29 g (50% of theory) of a mixture of tert-butyl (3RS,4RS)- and (3SR,4SR)-3-[2′[(RS)-(2,3-dihydroxy-propylcarbamoyl]-biphenyl-4-ylmethoxy]-4-(4-fluoro-phenyl)-piperidine-1-carboxylate as an amorphous, colorless foam; MS: 579 (M+H)⁺.

(d) In an analogous manner to that described in Example 22 (l), from a mixture of tert-butyl (3RS,4RS)- and (3SR,4SR)-3-[2′[(RS)-(2,3-dihydroxy-propylcarbamoyl]-biphenyl-4-ylmethoxy]-4-(4-fluoro-phenyl)-piperidine-1-carboxylate by cleavage of the BOC protecting group with hydrogen chloride in methanol there was obtained a mixture of 4′-[(3RS,4RS)-4-(4-fluoro-phenyl)-piperidin-3-yloxymethyl]-biphenyl-2-carboxylic acid (RS)- and (SR)-(2,3-dihydroxy-propyl)-amide hydrochloride (1:1) as an amorphous, colorless foam: MS: 479 (M+H)⁺.

### Example 93

(a) 0.050 g (0.12 mmol) of a 3:1 mixture of methyl (E)- and (Z)-[1-[(3RS,4SR)-4-(4-fluoro-phenyl)-piperidin-3-yl]-2-naphthalen-2-yl-ethylideneaminooxy]-acetate [Example 90.03] was stirred at 50° C. for 18 hours in 3 ml of methanol with the addition of 1.0 ml of 1 N sodium hydroxide solution. After cooling to room temperature 1.0 ml of 1 N hydrochloric acid was added dropwise and thereafter the mixture was concentrated in a water-jet vacuum. The residue was suspended in ethanol, then filtered, 0.025 ml (0.3 mmol) of hydrochloric acid (37%) was added to the filtrate and the mixture was again concentrated in a water-jet vacuum. This residue was now dried at room temperature in a high vacuum for 2 hours. There was thus obtained 0.040 g (73% of theory) of a mixture of (E)- and (Z)-(3RS,4SR)-(1-[4-(4-fluoro-phenyl)-piperidin-3-yl]-2-naphthalen-2-yl-et hylideneaminooxy)-acetic acid hydrochloride (1:1) as an amorphous, pale yellow foam; MS: 421 (M+H)⁺.

### Example 94

(a) 4.30 g (9.7 mmol) of tert-butyl (3RS,4RS)-4-[4-(3-benzyl-oxy-propoxy)-phenyl]-3-hydroxy-piperidine-1-carboxylate [Example 86 (n)] were placed in 215 ml of tetrahydrofuran at 0° C. under argon and 1.23 g (28.1 mmol) of sodium hydride dispersion (55% in mineral oil) were added while stirring. After 30 minutes a solution of 1.86 ml (12.7 mmol) of tert-butyl bromoacetate in 10 ml of tetrahydrofuran were added dropwise and the mixture was warmed to room temperature. The reaction mixture was poured on to ice-water, the product was extracted 3 times with ethyl acetate, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 4.77 g (88% of theory) of tert-butyl (3RS,4RS)-4-[4-(3-benzyloxy-propoxy)-phenyl]-3-tert-butoxycarbonylmethoxy-piperidine-1-carboxylate as a yellow oil; MS: 556 (M+H)⁺.

(b) 0.36 g (16.5 mmol) of lithium borohydride was placed in 55 ml of tetrahydrofuran at room temperature under argon and a solution of 4.58 g (8.24 mmol) of tert-butyl (3RS,4RS)-4-[4-(3-benzyloxy-propoxy)-phenyl]-3-tert-butoxycarbonylmethoxy-piperidine-1-carboxylate in 55 ml of tetrahydrofuran was added dropwise while stirring and the mixture was subsequently heated under reflux. After 4 hours the reaction mixture was poured on to ice-water, adjusted to pH 3 with hydrochloric acid (2 N) and then the product was extracted 3 times with ethyl acetate. The organic phases were thereupon washed twice with distilled water, dried over magnesium sulfate, filtered and concentrated in a water-jet vacuum. There were thus obtained 3.95 g (99% of theory) of tert-butyl (3RS,4RS)-4-[4-(3-benzyloxy-propoxy)-phenyl]-3-(2-hydroxy-ethoxy)-piperidine-1-carboxylate as a yellow oil; MS: 486 (M+H)⁺.

(c) 5.56 g (13.2 mmol) of triphenylphosphine dibromide were dissolved in 20 ml of acetonitrile under argon, then 1.06 ml (13.2 mmol) of pyridine were added dropwise at 0° C.; this solution was added dropwise to a solution of 3.95 g (8.1 mmol) of tert-butyl (3RS,4RS)-4-[4-(3-benzyloxy-propoxy)-phenyl]-3-(2-hydroxy-ethoxy)-piperidine-1-carboxylate in 20 ml of acetonitrile at 0° C. and subsequently the mixture was stirred at room temperature for 18 hours. The reaction mixture was poured on to ice-water, the product was then extracted 3 times with ethyl acetate, the organic phases were washed twice with distilled water, then dried over magnesium sulfate, filtered and concentrated in a water-jet vacuum. The thus-obtained crude product was chromatographed on silica gel with n-hexane and ethyl acetate. There were thus obtained 3.14 g (71% of theory) of tert-butyl (3RS,4RS)-4-[4-(3-benzyloxy-propoxy)-phenyl]-3-(2-bromo-ethoxy)-piperidine-1-carboxylate as a yellow oil; MS: 548, 550 (M+H)⁺.

(d) 3.14 g (5.72 mmol) of tert-butyl (3RS,4RS)-4-[4-(3-benzyloxy-propoxy)-phenyl]-3-(2-bromo-ethoxy)-piperidine-1-carboxylate and 2.53 g (13.76 mmol) of 3,4-dinitrophenol in 230 ml of acetonitrile were stirred at reflux under argon for 22 hours with the addition of 7.9 g (57.2 mmol) of potassium carbonate (anhydrous). After distillation of the solvent in a water-jet vacuum the residue was poured on to ice-water and the product was extracted 3 times with methylene chloride, the organic phases were washed twice with distilled water, then dried over magnesium sulfate, filtered and concentrated in a water-jet vacuum. The thus-obtained crude product was chromatographed on silica gel with n-hexane and ethyl acetate. There were thus obtained 2.57 g (69% of theory) of tert-butyl (3RS,4RS)-4-[4-(3-benzyloxy-propoxy)-phenyl]-3-[2-(3,4-dinitro-phenoxy)-ethoxy]-piperidine-1-carboxylate as a brown oil; MS: 652 (M+H)⁺.

(e) 1.63 g (2.5 mmol) of tert-butyl (3RS,4RS)-4-[4-(3-benzyloxy-propoxy)-phenyl]-3-[2-(3,4-dinitro-phenoxy)-ethoxy]-piperidine-1-carboxylate were hydrogenated under normal conditions for 2 hours in 80 ml of ethyl acetate with the addition of 0.50 g of platinum oxide. The catalyst was filtered off over a Dicalite pad and the solvent was distilled off in a water-jet vacuum. There were thus obtained 1.44 g (97% of theory) of tert-butyl (3RS,4RS)-4-[4-(3-benzyloxy-propoxy)-phenyl]-3-[2-(3,4-diamino-phenoxy)-ethoxy]-piperidine-1-carboxylate as a violet oil; MS: 592 (M+H)⁺.

(f) In an analogous manner to that described in Example 22 (l), from tert-butyl (3RS,4RS)-4-[4-(3-benzyloxy-propoxy)-phenyl]-3-[2-(3,4-diamino-phenoxy)-ethoxy]-piperidine-1-carboxylate by cleavage of the BOC protecting group with hydrogen chloride in methanol there was obtained 4-[2-[(3RS,4RS)-4-[4-(3-benzyloxy-propoxy)-phenyl]-piperidin-3-yloxy]-ethoxy]-benzene-1,2-diamine hydrochloride (1:3) as pale violet crystals; MS: 492 (M+H)⁺.

### Example 95

(a) 4.37 g (9.9 mmol) of (3RS,4RS)-4-[4-(3-benzyloxy-propoxy)-phenyl]-3-hydroxy-piperidine-1-carboxylate [Example 86 (n)] and 3.20 g (9.9 mmol) of 2-chloromethyl-7-(2-trimethylsilanyl-ethoxymethoxy)-naphthalene [Example 6 (u)] were dissolved in 35 ml of dimethylformamide under argon and then 0.46 g (10.5 mmol) of sodium hydride dispersion (55% in mineral oil) was added. Subsequently, the mixture was stirred at room temperature for 18 hours. The reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 7.15 g (99% of theory) of tert-butyl (3RS,4RS)-4-[4-(3-benzyloxy-propoxy)-phenyl]-3-[7-(2-trimethylsilanyl-ethoxymethoxy)-naphthalen-2-ylmethoxy]-piperidine-1-carboxylate as a pale yellow oil; MS: 728 (M+H)⁺.

(b) 6.72 g (9.23 g) of tert-butyl (3RS,4RS)-4-[4-(3-benzyloxy-propoxy)-phenyl]-3-[7-(2-trimethylsilanyl-ethoxymethoxy)-naphthalen-2-ylmethoxy]-piperidine-1-carboxylate were placed in 140 ml of abs. methanol at 0° C., then 2.8 ml (19.4 mmol) of hydrochloric acid in methanol (7.0 molar) were added dropwise at 5° C. max. and thereafter the mixture was warmed to room temperature. After 90 minutes the reaction mixture was poured into ice-cold sodium hydrogen carbonate solution and the product was extracted three times with methylene chloride, the organic phases were washed once with distilled water, then dried over magnesium sulfate, filtered and concentrated in a water-jet vacuum. The thus-obtained crude product was chromatographed on silica gel with methylene chloride and methanol. There were thus obtained 4.92 g (89% of theory) of tert-butyl (3RS,4RS)-4-[4-(3-benzyloxy-propoxy)-phenyl]-3-(7-hydroxy-naphthalen-2-ylmethoxy)-piperidine-1-carboxylate as a yellow oil; MS: 598 (M+H)⁺.

(c) In an analogous manner to that described in Example 22 (l), from tert-butyl (3RS,4RS)-4-[4-(3-benzyloxy-propoxy)-phenyl]-3-(7-hydroxy-naphthalen-2-ylmethoxy)-piperidine-1-carboxylate by cleavage of the BOC protecting group with hydrogen chloride in methanol there was obtained 7-[(3RS,4RS)-4-[4-(3-benzyloxy-propoxy)-phenyl]-piperidine-3-yloxymethyl]-naphthalen-2-ol hydrochloride (1:1) as an amorphous, beige foam; MS: 498 (M+H)⁺.

### Example 96

(a) 0.33 g (0.54 mmol) of tert-butyl (3RS,4RS)-4-[4-(3-benzyloxy-propoxy)-phenyl]-3-(7-hydroxy-naphthalen-2-ylmethoxy)-piperidine-1-carboxylate [Example 95 (b)] and 0.27 g (0.7 mmol) of di-O-isopropylidene-1-O-(4-methyl-phenyl-sulfonyl)-D-arabinitol [Liebigs Ann. Chem. 1992, 1131] were stirred at reflux under argon for 2 hours in 15 ml of dimethyl-formamide with the addition of 0.69 g (5 mmol) of potassium carbonate (anhydrous). The reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.34 g (78% of theory) of a 1:1 mixture of tert-butyl (3R,4R)- and (3S,4S)-4-[4-(3-benzyloxy-propoxy)-phenyl]-3-[7-[(4S,4′R,5R)-2,2,2′,2′-tetramethyl-[4,4′]bi[[1,3]dioxolan-5-ylmethoxy]-naphthalen-2-ylmethoxy]-piperidine-1-carboxylate as a colorless oil; MS: 829 (M+NH₄)⁺.

(b) 0.10 g (0.12 mmol) of a 1:1 mixture of tert-butyl (3R,4R)- and (3S,4S)-4-[4-(3-benzyloxy-propoxy)-phenyl]-3-[7-[(4S,4′R,5R)-2,2,2′,2′-tetramethyl-[4,4′]bi[[1,3]dioxolan-5-ylmethoxy]-naphthalen-2-ylmethoxy]-piperidine-1-carboxylate was dissolved in 5 ml of abs. ethanol, 1 ml of hydrochloric acid in ethanol (5.6 molar) was added thereto and the mixture was stirred at 50° C. under argon for 90 hours. After distillation of the solvent in a water-jet vacuum the residue was dried over phosphorus pentoxide at 50° C. in a high vacuum for 3 hours. There was thus obtained 0.07 g (87% of theory) of a 1:1 mixture of (2R,3R,4R)-5-[7-[(3R,4R)- and (3S,4S)-4-[4-(3-benzyloxy-propoxy)-phenyl]-piperidin-3-yloxymethyl]-naphthalen-2-yloxy]-pentane-1,2,3,4-tetraol hydrochloride (1:1) in the form of pale yellow crystals; MS: 632 (M+H)⁺.

### Example 97

(a) 4.77 g (10.8 mmol) of tert-butyl (3RS,4RS)-4-[4-(3-benzyloxy-propoxy)-phenyl]-3-hydroxy-piperidine-1-carboxylate [Example 86 (n)] and 3.49 g (10.8 mmol) of 2-chloromethyl-6-(2-trimethylsilanyl-ethoxymethoxy)-naphthalene [Example 6 (O)] were dissolved in 35 ml of dimethylformamide at room temperature under argon, then 0.50 g (11.5 mmol) of sodium hydride dispersion (55% in mineral oil) was added and the mixture was stirred at room temperature for 18 hours. The reaction mixture was poured on to ice-water, the product was extracted three times with methylene chloride, the organic phases were washed twice with distilled water, then dried over magnesium sulfate, filtered and concentrated in a water-jet vacuum. The thus-obtained crude product was chromatographed on silica gel with n-hexane and methylene chloride. There were thus obtained 6.74 g (83% of theory) of tert-butyl (3RS,4RS)-4-[4-(3-benzyloxy-propoxy)-phenyl]-3-[6-(2-trimethylsilanyl-ethoxymethoxy)-naphthalen-2-ylmethoxy]-piperidine-1-carboxylate as a pale yellow oil: MS: 728 (M+H)⁺.

(b) In an analogous manner to that described in Example 95 (b), from tert-butyl (3RS,4RS)-4-[4-(3-benzyloxy-propoxy)-phenyl]-3-[6-(2-trimethylsilanyl-ethoxymethoxy)-naphthalen-2-ylmethoxy]-piperidine-1-carboxylate there was obtained tert-butyl (3RS,4RS)-4-[4-(3-benzyloxy-propoxy)-phenyl]-3-(6-hydroxy-naphthalen-2-ylmethoxy)-piperidine-1-carboxylate as an amorphous, colorless foam; MS: 598 (M+H)⁺.

(c) In an analogous manner to that described in Example 90 (g), from tert-butyl (3RS,4RS)-4-[4-(3-benzyloxy-propoxy)-phenyl]-3-(6-hydroxy-naphthalen-2-ylmethoxy)-piperidine-1-carboxylate there was obtained tert-butyl (3RS,4RS)-4-[4-(3-benzyloxy-propoxy)-phenyl]-3-[6-(2-morpholin-4-yl-ethoxy)-naphthalen-2-ylmethoxy]-piperidine-1-carboxylate as a pale yellow oil; MS: 711 (M+H)⁺.

(d) In an analogous manner to that described in Example 22 (l), from tert-butyl (3RS,4RS)-4-[4-(3-benzyloxy-propoxy)-phenyl]-3-[6-(2-morpholin-4-yl-ethoxy)-naphthalen-2-ylmethoxy]-piperidine-1-carboxylate by cleavage of the BOC protecting group with hydrogen chloride in methanol there was obtained 4-[2-[6-[(3RS,4RS)-4-[4-(3-benzyloxy-propoxy)-phenyl]-piperidin-3-yloxymethyl]-naphthalen-2-yloxy]-ethyl]-morpholine hydrochloride (1:2) in the form of colorless crystals; M: 611 (M+H)⁺.

### Example 98

(a) 4.10 g (21.5 mmol) of p-toluenesulfonyl chloride were placed in 20 ml of abs. pyridine at 5° C. under argon, 0.06 g (0.5 mmol) of 4-dimethylaminopyridine was added and a solution of 3.58 g (20.3 mmol) of (RS)-2-[(2,2-dimethyl-1,3-dioxolan-4-yl)methoxy]-ethanol [J. Chem. Soc. 1965, 2968] in 20 ml of abs. pyridine was added dropwise while stirring. After stirring at room temperature for 6 hours the reaction mixture was poured on to ice-water, the product was extracted three times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 1.72 g (26% of theory) of (RS)-2-(2,2-dimethyl-[1,3]dioxolan-4-ylmethoxy)-ethyl toluene-4-sulfonate as a colorless oil; MS: 315 (M-CH₃).

(b) In an analogous manner to that described in Example 90 (g), from (RS)-2-(2,2-dimethyl-[1,3]dioxolan-4-ylmethoxy)-ethyltoluene-4-sulfonate and tert-butyl (3RS,4RS)-4-[4-(3-benzyloxy-propoxy)-phenyl]-3-(7-hydroxy-naphthalen-2-ylmethoxy)-piperidine-1-carboxylate [Example 95 (b)] there was obtained a mixture of tert-butyl (3RS,4RS)- and (3SR,4SR)-4-[4-(3-benzyloxy-propoxy)-phenyl]-3-[7-[2-[(RS)-2,2-dimethyl-[1,3]dioxolan-4-ylmethoxy]-ethoxy]-naphthalen-2-ylmethoxy]-piperidine-1-carboxylate in the form of colorless crystals; MS: 773 (M+NH₄)⁺.

(c) In an analogous manner to that described in Example 22 (l), from a mixture of tert-butyl (3RS,4RS)- and (3SR,4SR)-4-[4-(3-benzyloxy-propoxy)-phenyl]-3-[7-[2-[(RS)-2,2-dimethyl-[1,3]-dioxolan-4-ylmethoxy]-ethoxy]-naphthalen-2-ylmethoxy]-piperidine-1-carboxylate by cleavage of the BOC protecting group with hydrogen chloride in methanol there was obtained a mixture of (RS)- and (SR)-3-[2-[7-[(3RS,4RS)-4-[4-(3-benzyloxy-propoxy)-phenyl]-piperidin-3-yloxymethyl]-naphthalen-2-yloxy]-ethoxy]-propane-1,2-diol hydrochloride (1:1) as an amorphous, beige foam; MS: 616 (M+H)⁺.

### Example 99

a) 9.92 g (227.4 mmol) of sodium hydride dispersion (55% in mineral oil) were placed in 220 ml of abs. tetrahydrofuran at 5° C. under argon, a solution of 68.3 ml (341.1 mmol) of triethyl phosphonoacetate in 220 ml of abs. tetrahydrofuran was added dropwise thereto at 5° C. during 1 hour and the mixture was subsequently stirred at room temperature for 1 hour. Now, again at 5° C., a solution of 24.1 g (113.7 mmol) of 4-benzyloxy-benzaldehyde in 220 ml of tetrahydrofuran was added dropwise during 30 minutes and thereafter the mixture was stirred at 5° C. for 2 hours. The reaction mixture was treated with 300 ml of ice-water and the solvent was distilled off in a water-jet vacuum; the aqueous suspension of the product was extracted 3 times with ethyl acetate, the organic phases were washed twice with distilled water, then dried over magnesium sulfate, filtered and concentrated in a water-jet vacuum. The thus-obtained crude product was chromatographed on silica gel with n-hexane and ethyl acetate. There were thus obtained 30.8 g (96% of theory) of ethyl (E)-3-(4-benzyloxy-phenyl)-acrylate as a colorless solid; MS: 282 (M)⁺.

(b) 17.85 g (136.1 mmol) of malonic acid monoethyl ester monoamide in 350 ml of abs. ethanol were treated under argon with 15.3 g (136.1 mmol) of potassium tert-butylate, then 19.2 g (68.1 mmol) of ethyl (E)-3-(4-benzyloxy-phenyl)-acrylate were added at room temperature while stirring and the mixture was stirred at reflux for 1 hour. After cooling to 10° C. 15.4 ml (269.7 mmol) of glacial acetic acid were added dropwise. The reaction mixture was poured on to ice-water, the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 17.2 g (69% of theory) of ethyl (3RS,4SR)-4-(4-benzyloxy-phenyl)-2,6-dioxo-piperidine-3-carboxylate as a colorless solid; MS: 367 (M)⁺.

(c) 4.33 g (114.2 mmol) of lithium aluminium hydride were suspended in 200 ml of tetrahydrofuran under argon, then a solution of 18.31 g (49.8 mmol) of ethyl (3RS,4SR)-4-(4-benzyloxy-phenyl)-2,6-dioxo-piperidine-3-carboxylate in 200 ml of tetrahydrofuran was added dropwise at room temperature and the mixture was subsequently stirred at reflux for 2 hours. 100 ml of distilled water were cautiously added dropwise to the reaction mixture at 5-10° C. and the precipitate which thereby formed was filtered off. The filtrate was thereupon extracted 3 times with ethyl acetate, the organic phases were washed twice with distilled water, dried over magnesium sulfate, filtered and concentrated in a water-jet vacuum. The thus-obtained crude product was recrystallized from methylene chloride and n-hexane. There were thus obtained 11.14 g (75% of theory) of (3RS,4SR)-[4-(4-benzyloxy-phenyl)-piperidin-3-yl]-methanol in the form of colorless crystals; MS: 297 (M)⁺.

(d) 11.14 g (37.5 mmol) of (3RS,4SR)-[4-(4-benzyloxy-phenyl)-piperidin-3-yl]-methanol were dissolved in 140 ml of dioxan under argon, then a solution of 6.72 g (80 mmol) of sodium hydrogen carbonate in 45 ml of water was added at room temperature and 9.78 g (44.8 mmol) of di-tert-butyl dicarbonate were introduced portionwise. After stirring at room temperature for 18 hours the reaction mixture was extracted 3 times with ethyl acetate, the organic phases were washed twice with distilled water, then dried over magnesium sulfate, filtered and concentrated in a water jet vacuum. The thus-obtained crude product was chromatographed on silica gel with methylene chloride and methanol. There were thus obtained 13.38 g (90% of theory) of tert-butyl (3RS,4SR)-4-(4-benzyloxy-phenyl)-3-hydroxymethyl-piperidine-1-carboxylate as an amorphous, colorless foam; MS: 398 (M+H)⁺.

(e) 3.92 ml (45.6 mmol) of oxalyl chloride were placed in 400 ml of methylene chloride at −70° C. under argon, 5.48 ml (77.2 mmol) of dimethyl sulfoxide were added dropwise thereto and the mixture was stirred for 30 minutes. Now, a solution of 13.95 g (35.1 mmol) of tert-butyl (3RS,4SR)-4-(4-benzyloxy-phenyl)-3-hydroxymethyl-piperidine-1-carboxylate in 200 ml of methylene chloride was added dropwise at −70° C. and thereafter the mixture was stirred at this temperature for 2 hours. Subsequently, 12.2 ml (87.7 mmol) of triethylamine were added dropwise to the reaction mixture and thereafter it was warmed to room temperature. After stirring at this temperature for 18 hours the mixture was poured on to ice-water and the product was extracted 3 times with methylene chloride, the organic phases were washed twice with distilled water, then dried over magnesium sulfate, filtered and concentrated in a water-jet vacuum. The thus-obtained crude product was recrystallized from n-hexane. There were thus obtained 11.31 g (81% of theory) of tert-butyl (3RS,4SR)-4-(4-benzyloxy-phenyl)-3-formyl-piperidine-1-carboxylate in the form of colorless crystals; MS: 395 (M)⁺.

(f) 11.04 g (25.6 mmol) of tributyl-naphthalen-2-yl-stannate [Example 74 (g)] were placed in 100 ml of tetrahydrofuran at −70° C. under argon and 12.0 ml (19.2 mmol) of n-butyllithium solution (1.6 molar in n-hexane) were added dropwise thereto. After stirring at this temperature for 30 minutes a solution of 5.94 g (15 mmol) of tert-butyl (3RS,4SR)-4-(4-benzyloxy-phenyl)-3-formyl-piperidine-1-carboxylate in 45 ml of tetrahydrofuran was added dropwise and the mixture was stirred at −70° C. for a further 1 hour. Now, the mixture was warmed to room temperature and, after 18 hours, poured on to ice-water and the product was extracted 3 times with ethyl acetate; the organic phases were washed twice with distilled water, then dried over magnesium sulfate, filtered and concentrated in a water-jet vacuum. The thus-obtained crude product was chromatographed on silica gel with n-hexane and ethyl acetate. There were thus obtained 6.88 g (85% of theory) of a mixture of tert-butyl (3RS,4SR)-4-(4-benzyloxy-phenyl)-3-[(RS)- and -[(SR)-1-hydroxy-2-naphthalen-2-yl-ethyl]-piperidine-1-carboxylate in the form of yellow crystals; MS: 538 (M+H)⁺.

(g) In an analogous manner to that described in Example 22 (l), from a mixture of tert-butyl (3RS,4SR)-4-(4-benzyloxy-phenyl)-3-[(RS)- and -[(SR)-1-hydroxy-2-naphthalen-2-yl-ethyl]-piperidine-1-carboxylate by cleavage of the BOC protecting group with hydrogen chloride in methanol there was obtained a mixture of (RS)- and (SR)-1-[(3RS,4SR)-4-(4-benzyloxy-phenyl)-piperidin-3-yl]-2-naphthalen-2-yl-ethanol hydrochloride (1:1) in the form of beige crystals; MS: 438 (M+H)⁺.

### Example 100

(a) 6.36 g (11.8 mmol) of a mixture of tert-butyl (3RS,4SR)-4-(4-benzyloxy-phenyl)-3-[(RS)- and -[(SR)-1-hydroxy-2-naphthalen-2-yl-ethyl)-piperidine-1-carboxylate [Example 99 (f)] were hydrogenated under normal conditions for 4 hours in 50 ml of methanol with the addition of 2.0 g of palladium-charcoal (10%). After filtration of the catalyst over a Dicalite pad and distillation of the solvent in a water-jet vacuum there were obtained 4.97 g (94% of theory) of a mixture of tert-butyl (3RS,4SR)-4-(4-hydroxy-phenyl)-3-[(RS)-and-(SR)-1-hydroxy-2-naphthalen-2-yl-ethyl)-piperidine-1-carboxylate as an amorphous, pale yellow foam; MS: 448 (M+H)⁺.

(b) 3.97 g (8.9 mmol) of a mixture of tert-butyl (3RS,4SR)-4-(4-hydroxy-phenyl)-3-[(RS)- and -(SR)-1-hydroxy-2-naphthalen-2-yl-ethyl]-piperidine-1-carboxylate were placed in 60 ml of methyl ethyl ketone at room temperature under argon, then 4.90 g (35.5 mmol) of potassium carbonate (anhydrous) and 4.54 ml (27.7 mmol) of benzyl 3-bromo-propyl ether were added and thereafter the mixture was stirred at reflux for 8 hours. After cooling to room temperature the reaction mixture was poured on to ice-water and the product was extracted 3 times with ethyl acetete, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 5.13 g (97% of theory) of a mixture of tert-butyl (3RS,4SR)-4-[4-(3-benzyloxy-propoxy)-phenyl]-3-[(RS)- and -[(SR)1-hydroxy-2-naphthalen-2-yl-ethyl]-piperidine-1-carboxylate as an amorphous yellow foam; MS: 596 (M+H)⁺.

(c) In an analogous manner to that described in Example 99 (e), from a mixture of tert-butyl (3RS,4SR)-4-[4-(3-benzyloxy-propoxy)-phenyl]-3-[(RS)- and -[(SR)1-hydroxy-2-naphthalen-2-yl-ethyl]-piperidine-1-carboxylate with oxalyl chloride and dimethyl sulfoxide there was obtained tert-butyl (3RS,4SR)-4-[4-(3-benzyloxy-propoxy)-phenyl]-3-(naphthalen-2-yl-acetyl)-piperidine-1-carboxylate as a colorless oil; MS: 594 (M+H)⁺.

(d) In an analogous manner to that described in Example 22 (l), from tert-butyl 3RS,4SR)-4-[4-(3-benzyloxy-propoxy)-phenyl]-3-(naphthalen-2-yl-acetyl)-piperidine-1-carboxylate by cleavage of the BOC protecting group with hydrogen chloride in methanol there was obtained [(3RS,4SR)-4-[4-(3-benzyloxy-propoxy)-phenyl]-piperidin-3-yl]-2-naphthalen-2-yl-ethanone hydrochloride (1:1) in in the form of colorless crystals; MS: 494 (M+H)⁺.

### Example 101

(a) 10.66 g (50.2 mmol) of 4-benzyloxy-benzaldehyde and 8.54 ml (56.2 mmol) of diethyl malonate were stirred at reflux under argon for 18 hours in 100 ml of toluene with the addition of 10.15 g of molecular sieve (4 Å), 1.0 ml (10.0 mmol) of piperidine and 1.0 ml (17.6 mmol) of glacial acetic acid. After filtration of the reaction mixture the solvent was distilled off in a water-jet vacuum and the residue was chromatographed on silica gel with n-hexane and ethyl acetate. There were thus obtained 14.05 g (83% of theory) of diethyl 2-(4-benzyloxy-benzylidene)-malonate in the form of yellow crystals; MS: 354 (M)⁺.

(b) 6.42 g (48.9 mmol) of malonic acid monoethyl ester mono-amide in 115 ml of abs. ethanol were treated with 5.49 g (48.9 mmol) of potassium tert-butylate under argon, then 17.35 g (48.9 mmol) of diethyl 2-(4-benzyloxy-benzylidene)-malonate were added at room temperature while stirring and the mixture was stirred at reflux for 2 hours. After cooling to 10° C. 13.0 ml (227 mmol) of glacial acetic acid were added dropwise. The reaction mixture was poured on to ice-water, the product was extracted three times with methylene chloride, the organic phases were washed twice with distilled water, then dried over magnesium sulfate, filtered and concentrated in a water-jet vacuum. The thus-obtained crude product was recrystallized from methylene chloride and n-hexane. There were thus obtained 16.76 g (78% of theory) of diethyl (3R,4s,5S)-4-(4-benzyloxy-phenyl)-2,6-dioxo-piperidine-3,5-dicarboxylate as a colorless solid; MS: 439 (M)⁺.

(c) 3.83 g (100.9 mmol) of lithium aluminium hydride were suspended in 200 ml of tetrahydrofuran under argon, then a solution of 18.37 g (41.8 mmol) of diethyl (3R,4s,5S)-4-(4-benzyloxy-phenyl)-2,6-dioxo-piperidine-3,5-dicarboxylate in 200 ml of tetrahydrofuran was added dropwise and the mixture was subsequently stirred at reflux for 1 hour. Subsequently, 25 ml of distilled water were cautiously added dropwise to the reaction mixture at 5-10° C. After filtration of the reaction mixture the solvent was distilled off in a water-jet vacuum. There were thus obtained 11.04 g (81% of theory) of (3R,4s,5S)-[4-(4-benzyloxy-phenyl)-5-hydroxymethyl-piperidin-3-yl]-methanol in the form of colorless crystals; MS: 328 (M+H)⁺.

(d) 8.10 g (24.7 mmol) of (3R,4s,5S)-[4-(4-benzyloxy-phenyl)-5-hydroxymethyl-piperidin-3-yl)-methanol were dissolved in 100 ml of dioxan under argon, then a solution of 4.44 g (52.8 mmol) of sodium hydrogen carbonate in 34 ml of water was added dropwise at room temperature and subsequently 6.46 g (29.6 mmol) of di-tert-butyl dicarbonate were introduced portionwise. After stirring at room temperature for 66 hours the reaction mixture was extracted 3 times with ethyl acetate, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 8.10 g (77% of theory) of tert-butyl (3R,4s,5S)-4-(4-benzyloxy-phenyl)-3,5-bis-hydroxymethyl-piperidine-1-carboxylate as an amorphous, colorless foam; MS: 428 (M+H)⁺.

(e) 7.46 g (17.5 μmmol) of tert-butyl (3R,4s,5S)-4-(4-benzyloxy-phenyl)-3,5-bis-hydroxymethyl-piperidine-1-carboxylate were hydrogenated under normal conditions for 2 hours in 250 ml of methanol with the addition of 1.5 g of palladium-charcoal (10%). The catalyst was subsequently filtered off over a Dicalite pad and the solvent was distilled off in a water-jet vacuum. The thus-obtained crude product was chromatographed on silica gel with methylene chloride and methanol. There were thus obtained 6.07 g (99% of theory) of tert-butyl (3R,4s,5S)-3,5-bis-hydroxymethyl-4-(4-hydroxy-phenyl)-piperidine-1-carboxylate as an amorphous, colorless foam; MS: 338 (M+H)⁺.

(f) 6.77 g (20 mmol) of tert-butyl (3R,4s,5S)-3,5-bis-hydroxymethyl-4-(4-hydroxy-phenyl)-piperidine-1-carboxylate were placed in 90 ml of methyl ethyl ketone at room temperature under argon. Thereupon, 11.05 g (80 mmol) of potassium carbonate (anhydrous) and 10.25 ml (58 mmol) of benzyl 3-bromo-propyl ether were added and thereafter the mixture was stirred at reflux for 18 hours. After cooling to room temperature the reaction mixture was poured on to ice-water and the product was extracted 3 times with ethyl acetate, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 7.95 g (82% of theory) of tert-butyl (3R,4s,5S)-4-[4-(3-benzyloxy-propoxy)-phenyl]-3,5-bis-hydroxymethyl-piperidine-1-carboxylate as an amorphous, colorless foam; MS: 486 (M+H)⁺.

(g) 1.51 g (33.2 mmol) of sodium hydride dispersion (55% in mineral oil) and 2.94 g (15.9 mmol) of 4-(2-chloroethyl)-morpholine hydrochloride were dissolved in 25 ml of dimethyl-formamide under argon, a solution of 7.33 g (15.1 mmol) of tert-butyl (3R,4s,5S)-4-[4-(3-benzyloxy-propoxy)-phenyl]-3,5-bis-hydroxymethyl-piperidine-1-carboxylate in 50 ml of dimethyl-formamide was added dropwise while stirring and 0.1 g (0.6 mmol) of potassium iodide was added. The reaction mixture was stirred at 100° C. for 9 hours. After cooling to room temperature the reaction mixture was poured on to ice-water and the product was extracted 3 times with methylene chloride, the organic phases were washed twice with distilled water, then dried over magnesium sulfate, filtered and concentrated in a water-jet vacuum. The thus-obtained crude product was chromatographed on silica gel with methylene chloride and methanol. There were thus obtained 0.29 g (3% of theory) of tert-butyl (3R,4S,5S)-4-[4-(3-benzyloxy-propoxy)-phenyl]-3,5-bis-(2-morpholin-4-yl-ethoxymethyl)-piperidine-1-carboxylate as a pale brown oil [MS: 712 (M+H)⁺] and 2.37 g (26% of theory) of tert-butyl (3RS,4RS,5SR)-4-[4-(3-benzyloxy-propoxy)-phenyl]-3-hydroxymethyl-5-(2-morpholin-4-yl-ethoxymethyl)-piperidine-1-carboxylate as a pale brown oil; MS: 599 (M+H)⁺.

(h) In an analogous manner to that described in Example 74 (f), from tert-butyl (3RS,4RS,5SR)-4-[4-(3-benzyloxy-propoxy)-phenyl]-3-hydroxymethyl-5-(2-morpholin-4-yl-ethoxymethyl)-piperidine-1-carboxylate by oxidation with dimethyl sulfoxide/oxalyl chloride in methylene chloride there was obtained tert-butyl (3RS,4SR,5SR)-4-[4-(3-benzyloxy-propoxy)-phenyl]-3-formyl-5-(2-morpholin-4-yl-ethoxymethyl)-piperidine-1-carboxylate as a yellow oil; MS: 597 (M+H)⁺.

(i) 1.20 g (0.05 g atom) of magnesium shavings were placed in 15 ml of abs. ether at room temperature under argon, 1 crystal of iodine and 5 drops of 1,2-dibromomethane were added and the mixture was heated to reflux. After commencement of the reaction (decoloration) a solution of 1.77 g (10 mmol) of 2-(chloromethyl)-naphthalene in 10 ml of abs. ether was added dropwise during 30 minutes. After the addition the mixture was left to cool to room temperature and after one hour a solution of 0.70 g (1.17 mmol) of tert-butyl (3RS,4SR,5SR)-4-[4-(3-benzyloxy-propoxy)-phenyl]-3-formyl-5-(2-morpholin-4-yl-ethoxymethyl)-piperidine-1-carboxylate in 15 ml of abs. ether was added dropwise. Then, the mixture was stirred at room temperature for 18 hours. After the dropwise addition of 3 ml of water while cooling with ice the reaction mixture was poured on to ice-water, the product was extracted 3 times with ether, the organic phases were washed twice with distilled water, then dried over magnesium sulfate, filtered and concentrated in a water-jet vacuum. The thus-obtained crude product was chromatographed on silica gel with methylene chloride and methanol. There was thus obtained 0.77 g (89% of theory) of a mixture of tert-butyl (3RS,4SR,5SR)-4-[4-(3-benzyloxy-propoxy)-phenyl]-3-[(RS)- and -[(SR)-1-hydroxy-2-naphthalen-2-yl-ethyl]-5-(2-morpholin-4-yl-ethoxymethyl)-piperidine-1-carboxylate; MS: 739 (M+H)⁺.

(k) In an analogous manner to that described in Example 74 (f), from 0.74 g (1 mmol) of a mixture of tert-butyl (3RS,4SR,5SR)-4-[4-(3-benzyloxy-propoxy)-phenyl]-3-[(RS)- and -[(SR)-1-hydroxy-2-naphthalen-2-yl-ethyl]-5-(2-morpholin-4-yl-ethoxyethyl)-piperidine-1-carboxylate by oxidation with dimethyl sulfoxide/oxalyl chloride in methylene chloride there was obtained 0.15 g (20% of theory) of tert-butyl (3RS,4SR,5SR)-4-[4-(3-benzyloxy-propoxy)-phenyl]-5-(2-morpholin-4-yl-ethoxymethyl)-3-(naphthalen-2-yl-acetyl)-piperidine-1-carboxylate as a pale yellow oil; MS: 737 (M+H)⁺.

(l) In an analogous manner to that described in Example 73 (d), from tert-butyl (3RS,4SR,5SR)-4-[4-(3-benzyloxy-propoxy)-phenyl]-5-(2-morpholin-4-yl-ethoxymethyl)-3-(naphthalen-2-yl-acetyl)-piperidine-1-carboxylate by cleavage of BOC protecting group by means of anhydrous zinc bromide in methylene chloride there was obtained 1-[(3RS,4SR,5SR)-4-[4-(3-benzyloxy-propoxy)-phenyl]-5-(2-morpholin-4-yl-ethoxymethyl)-piperidin-3-yl]-2-naphthalen-2-yl-ethanone as a yellow oil MS: 637 (M+H)⁺.

### Example 102

(a) 0.30 g (0.5 mmol) of tert-butyl (3RS,4SR)-4-[4-(3-benzyloxy-propoxy)-phenyl]-3-(naphthalen-2-yl-acetyl)-piperidine-1-carboxylate [Example 100 (c)] and 0.071 g (0.5 mmol) of 3-(aminooxy)propionic acid hydrochloride (J. Am. Chem. Soc. 77, 2345 (1955)] in 3 ml of pyridine were stirred at 60° C. under argon for 18 hours. The reaction mixture was thereupon poured on to ice-water, adjusted to pH 3 with dilute hydrochloric acid, then the product was extracted 3 times with ethyl acetate, the organic phases were washed twice with distilled water, dried over magnesium sulfate, filtered and concentrated in a water-jet vacuum. The thus-obtained crude product was chromatographed on silica gel with methylene chloride and methanol. There was thus obtained 0.018 g (5% of theory) of a mixture of tert-butyl (E)- and (Z)-(3RS,4SR)-4-[4-(3-benzyloxy-propoxy)-phenyl]-3-[1-(2-carboxy-ethoxyimino)-2-naphthalen-2-yl-ethyl]-piperidine-1-carboxylate as a pale yellow oil [MS: 681 (M+H)⁺] and 0.21 g (69% of theory) of a mixture of tert-butyl (E)- and (Z)-(3RS,4SR)-4-[4-(3-benzyloxy-propoxy)-phenyl]-3-(1-hydroxyimino-2-naphthalen-2-yl-ethyl)-piperidine-1-carboxylate as a colorless oil; MS: 609 (M+H)⁺.

(b) In an analogous manner to that described in Example 22 (l) from a mixture of tert-butyl (E)- and (Z)-(3RS,4SR)-4-[4-(3-benzyloxy-propoxy)-phenyl]-3-[1-(2-carboxy-ethoxyimino)-2-naphthalen-2-yl-ethyl]-piperidine-1-carboxylate by cleavage of the BOC protecting group with hydrogen chloride in dioxan there was obtained a mixture of (E)- and (Z)-3-(1-[(3RS,4SR)-4-[4-(3-benzyloxy-propoxy)-phenyl]-piperidin-3-yl]-2-naphthalen-2-yl-ethylidenaminooxy)-propionic acid as an amorphous, beige foam [MS: 581 (M+H)⁺] and from a mixture of tert-butyl (E)- and (Z)-(3RS,4SR)-4-[4-(3-benzyloxy-propoxy)-phenyl]-3-(1-hydroxyimino-2-naphthalen-2-yl-ethyl)-piperidine-1-carboxylate by cleavage of the BOC protecting group with hydrogen chloride in methanol there was a obtained a mixture of (E)- and (Z)-1-[(3RS,4SR)-4-[4-(3-benzyloxy-propoxy)-phenyl]-piperidin-3-yl]-2-naphthalen-2-yl-ethanone oxime as a colorless solid; MS: 509 (M+H)⁺.

### Example 103

(a) 0.15 g (0.22 mmol) of a mixture of tert-butyl (E)- and (Z)-(3RS,4SR)-4-[4-(3-benzyloxy-propoxy)-phenyl]-3-(1-methoxy-carbonyl-methoxyimino-2-naphthalen-2-yl-ethyl)-piperidine-1-carboxylate {Example 90 (p)] was dissolved in 10 ml of methanol, treated with 1.3 ml of 3.1 molar hydrochloric acid in methanol (4 mmol) and the reaction mixture was stirred at room temperature for 7 hours. After the addition of 1 ml (9 mmol) of sodium hydroxide solution (28%) at 5° C. the reaction mixture was stirred at room temperature for 18 hours. The pH value of the reaction solution was adjusted to 1 by the dropwise addition of 0.7 ml (8.75 mmol) of hydrochloric acid (37%), the mixture was filtered and the solvent was distilled off in a high vacuum. The residue was suspended in 5 ml of abs. ethanol, filtered and the filtrate was concentrated. The thus-obtained crude product was chromatographed on silica gel with methylene chloride, methanol and ammonia solution (25%). There was thus obtained 0.059 g (47% of theory) of a mixture of (E)- and (Z)-(1-[(3RS,4SR)-4-[4-(3-benzyloxy-propoxy)-phenyl]-piperidin-3-yl}-2-naphthalen-2-yl-ethylidene-aminooxy)-acetic acid as an amorphous, beige foam; MS: 567 (M+H)⁺.

### Example 104

The following compounds were obtained analogously to Example 1 (g) by synthesizing the corresponding BOC derivatives by alkylating of tert-butyl (3RS,4RS)-4-[4-(3-benzyloxy-propoxy)-phenyl]-3-hydroxy-piperidine-1-carboxylate [Example 86 (n)] and using these derivatives without further purification and characterization in the cleavage reaction of the BOC group by means of hydrogen chloride in methanol analogously to Example 22 (l) or by means of zinc bromide in methylene chloride analgously to Example 10(b):

1)—By alkylation with 4-trifluoromethyl-benzyl bromide and cleavage of the BOC group by means of hydrogen chloride in methanol, (3RS,4RS)-4-[4-(3-benzyloxy-propoxy)-phenyl]-3-(4-trifluoromethyl-benzyloxy)-piperidine hydrochloride as a colorless oil; MS: 500 (M+H)⁺;

2)—by alkylation with 4-fluoro-benzyl bromide and cleavage of the BOC group by means of hydrogen chloride in methanol, (3RS,4RS)-4-[4-(3-benzyloxy-propoxy)-phenyl]-3-(4-fluoro-benzyloxy)-piperidine hydrochloride as a colorless oil; MS: 450 (M+H)⁺;

3)—by alkylation with 2-chloro-benzyl bromide and cleavage of the BOC group by means of hydrogen chloride in methanol, (3RS,4RS)-4-[4-(3-benzyloxy-propoxy)-phenyl]-3-(2-chloro-benzyloxy)-piperidine hydrochloride as a colorless oil; MS: 466 (M)⁺;

4)—by alkylation with 4-bromo-benzyl bromide and cleavage of the BOC group by means of hydrogen chloride in methanol, (3RS,4RS)-4-[4-(3-benzyloxy-propoxy)-phenyl]-3-(4-bromo-benzyloxy)-piperidine hydrochloride as a colorless oil; MS: 510 (M)⁺;

5)—by alkylation with 3-bromo-benzyl bromide and cleavage of the BOC group by means of hydrogen chloride in methanol, (3RS,4RS)-4-[4-(3-benzyloxy-propoxy)-phenyl]-3-(3-bromo-benzyloxy)-piperidine hydrochloride as a colorless oil; MS: 511 (M+H)⁺;

6)—by alkylation with 4-iodo-benzyl bromide and cleavage of the BOC group by means of hydrogen chloride in methanol, (3RS,4RS)-4-[4-(3-benzyloxy-propoxy)-phenyl]-3-(4-iodo-benzyloxy)-piperidine hydrochloride as a colorless oil; MS: 558 (M+H)⁺;

7)—by alkylation with 2-trifluoromethyl-benzyl bromide and cleavage of the BOC group by means of hydrogen chloride in methanol, (3RS,4RS)-4-[4-(3-benzyloxy-propoxy)-phenyl]-3-(2-trifluoromethyl-benzyloxy)-piperidine hydrochloride as a colorless oil; MS: 500 (M+H)⁺;

8)—by alkylation with 3,5-dimethyl-benzyl bromide and cleavage of the BOC group by means of hydrogen chloride in methanol, (3RS,4RS)-4-[4-(3-benzyloxy-propoxy)-phenyl]-3-(3,5-dimethyl-benzyloxy)-piperidine hydrochloride as a colorless oil; MS: 460 (M+H)⁺;

9)—by alkylation with 2,4-dimethyl-benzyl chloride and cleavage of the BOC group by means of hydrogen chloride in methanol, (3RS,4RS)-4-[4-(3-benzyloxy-propoxy)-phenyl]-3-(2,4-dimethyl-benzyloxy)-piperidine hydrochloride as a colorless oil; MS: 460 (M+H)⁺;

10)—by alkylation with 4-methyl-benzyl bromide and cleavage of the BOC group by means of hydrogen chloride in methanol, (3RS,4RS)-4-[4-(3-benzyloxy-propoxy)-phenyl]-3-(4-methyl-benzyloxy)-piperidine hydrochloride as a colorless oil; MS: 446 (M+H)⁺;

11)—by alkylation with 4-isopropyl-benzyl chloride and cleavage of the BOC group by means of hydrogen chloride in methanol, (3RS,4RS)-4-[4-(3-benzyloxy-propoxy)-phenyl]-3-(4-isopropyl-benzyloxy)-piperidine hydrochloride as a colorless oil; MS: 474 (M+H)⁺;

12)—by alkylation with 4-tert-butyl-benzyl bromide and cleavage of the BOC group by means of hydrogen chloride in methanol, (3RS,4RS)-4-[4-(3-benzyloxy-propoxy)-phenyl]-3-(4-tert-butyl-benzyloxy)-piperidine hydrochloride as a colorless oil; MS: 488 (M+H)⁺;

13)—by alkylation with 2-methoxy-benzyl chloride and cleavage of the BOC group by means of hydrogen chloride in methanol, (3RS,4RS)-4-[4-(3-benzyloxy-propoxy)-phenyl]-3-(2-methoxy-benzyloxy)-piperidine hydrochloride as a colorless oil; MS: 462 (M+H)⁺;

14)—by alkylation with 2-fluoro-benzyl bromide and cleavage of the BOC group by means of hydrogen chloride in methanol, (3RS,4RS)-4-[4-(3-benzyloxy-propoxy)-phenyl]-3-(2-fluoro-benzyloxy)-piperidine hydrochloride as a colorless oil; MS: 450 (M+H)⁺;

15)—by alkylation with 2-fluoro-6-trifluoromethyl-benzyl bromide and cleavage of the BOC group by means of hydrogen chloride in methanol, (3RS,4RS)-4-[4-(3-benzyloxy-propoxy)-phenyl]-3-(2-fluoro-6-trifluoromethyl-benzyloxy)-piperidine hydrochloride as a colorless oil; MS: 518 (M+H)⁺;

16)—by alkylation with 2-bromo-5-fluoro-benzyl bromide and cleavage of the BOC group by means of hydrogen chloride in methanol, (3RS,4RS)-4-[4-(3-benzyloxy-propoxy)-phenyl]-3-(2-bromo-5-fluoro-benzyloxy)-piperidine hydrochloride as a colorless oil; MS: 528 (M)⁺;

17)—by alkylation with 4-fluoro-3-trifluoromethyl-benzyl bromide and cleavage of the BOC group by means of hydrogen chloride in methanol, (3RS,4RS)-4-[4-(3-benzyloxy-propoxy)-phenyl]-3-(4-fluoro-3-trifluoromethyl-benzyloxy)-piperidine hydrochloride as a colorless oil; MS: 518 (M+H)⁺;

18)—by alkylation with 3,5-di-trifluoromethyl-benzyl bromide and cleavage of the BOC group by means of hydrogen chloride in methanol, (3RS,4RS)-4-[4-(3-benzyloxy-propoxy)-phenyl]-3-(3,5-bis-trifluoromethyl-benzyloxy)-piperidine hydrochloride as a colorless oil; MS: 568 (M+H)⁺;

19)—by alkylation with 2-fluoro-3-methyl-benzyl bromide and cleavage of the BOC group by means of hydrogen chloride in methanol, (3RS,4RS)-4-[4-(3-benzyloxy-propoxy)-phenyl]-3-(2-fluoro-3-methyl-benzyloxy)-piperidine hydrochloride as a colorless oil; MS: 464 (M+H)⁺;

20)—by alkylation with 2-fluoro-4-trifluoromethyl-benzyl bromide and cleavage of the BOC group by means of hydrogen chloride in methanol, (3RS,4RS)-4-[4-(3-benzyloxy-propoxy)-phenyl)-3-(2-fluoro-4-trifluoromethyl-benzyloxy)-piperidine hydrochloride as a yellowish oil; MS: 518 (M+H)⁺;

21)—by alkylation with 2-fluoro-5-trifluoromethyl-benzyl bromide and cleavage of the BOC group by means of hydrogen chloride in methanol, (3RS,4RS)-4-[4-(3-benzyloxy-propoxy)-phenyl]-3-(2-fluoro-5-trifluoromethyl-benzyloxy)-piperidine hydrochloride as a yellowish oil; MS: 518 (M+H)⁺;

22)—by alkylation with 4-fluoro-2-trifluoromethyl-benzyl bromide and cleavage of the BOC group by means of hydrogen chloride in methanol, (3RS,4RS)-4-[4-(3-benzyloxy-propoxy)-phenyl]-3-(4-fluoro-2-trifluoromethyl-benzyloxy)-piperidine hydrochloride as a colorless oil; MS: 518 (M+H)⁺;

23)—by alkylation with 3,5-dichloro-benzyl chloride and cleavage of the BOC group by means of hydrogen chloride in methanol, (3RS,4RS)-4-[4-(3-benzyloxy-propoxy)-phenyl]-3-(3,5-dichloro-benzyloxy)-piperidine hydrochloride as a colorless oil; MS: 500 (M)⁺;

24)—by alkylation with 2,4-dichloro-benzyl chloride and cleavage of the BOC group by means of hydrogen chloride in methanol, (3RS,4RS)-4-[4-(3-benzyloxy-propoxy)-phenyl]-3-(2,4-dichloro-benzyloxy)-piperidine hydrochloride as a colorless oil; MS: 500 (M)⁺;

25)—by alkylation with 2-bromo-benzyl chloride and cleavage of the BOC group by means of hydrogen chloride in methanol, (3RS,4RS)-4-[4-(3-benzyloxy-propoxy)-phenyl]-3-(2-bromo-benzyloxy)-piperidine hydrochloride as a colorless oil; MS: 510 (M)⁺;

26)—by alkylation with 2,6-dichloro-benzyl chloride and cleavage of the BOC group by means of hydrogen chloride in methanol, (3RS,4RS)-4-[4-(3-benzyloxy-propoxy)-phenyl]-3-(2,6-dichloro-benzyloxy)-piperidine hydrochloride as a colorless oil; MS: 500 (M+H)⁺;

27)—by alkylation with 3-fluoro-benzyl chloride and cleavage of the BOC group by means of hydrogen chloride in methanol, (3RS,4RS)-4-[4-(3-benzyloxy-propoxy)-phenyl]-3-(3-fluoro-benzyloxy)-piperidine hydrochloride as a colorless oil; MS: 450 (M+H)⁺;

28)—by alkylation with 6-chloro-2-fluoro-benzyl chloride and cleavage of the BOC group by means of hydrogen chloride in methanol, (3RS,4RS)-4-[4-(3-benzyloxy-propoxy)-phenyl]-3-(2-chlor-6-fluoro-benzyloxy)-piperidine hydrochloride as a colorless oil; MS: 484 (M)⁺;

29)—by alkylation with 2-iodo-benzyl chloride and cleavage of the BOC group by means f hydrogen chloride in methanol, (3RS,4RS)-4-[4-(3-benzyloxy-propoxy)-phenyl]-3-(2-iodo-benzyloxy)-piperidine hydrochloride as a colorless oil; MS: 558 (M+H)⁺;

30)—by alkylation with 3,4-difluoro-benzyl bromide and cleavage of the BOC group by means of hydrogen chloride in methanol, (3RS,4RS)-4-[4-(3-benzyloxy-propoxy)-phenyl]-3-(3,4-difluoro-benzyloxy)-piperidine hydrochloride as a colorless oil; MS: 468 (M+H)⁺;

31)—by alkylation with 2,3-difluoro-benzyl bromide and cleavage of the BOC group by means of hydrogen chloride in methanol, (3RS,4RS)-4-[4-(3-benzyloxy-propoxy)-phenyl]-3-(2,3-difluoro-benzyloxy)-piperidine hydrochloride as a colorless oil; MS: 468 (M+H)⁺;

32)—by alkylation with 2,5-difluoro-benzyl bromide and cleavage of the BOC group by means of hydrogen chloride in methanol, (3RS,4RS)-4-[4-(3-benzyloxy-propoxy)-phenyl]-3-(2,5-difluoro-benzyloxy)-piperidine hydrochloride as a colorless oil; MS: 468 (M+H)⁺;

33)—by alkylation with 2,6-difluoro-benzyl chloride and cleavage of the BOC group by means of hydrogen chloride in methanol, (3RS,4RS)-4-[4-(3-benzyloxy-propoxy)-phenyl]-3-(2,6-difluoro-benzyloxy)-piperidine hydrochloride as a colorless oil; MS: 468 (M+H)⁺;

34)—by alkylation with 2,4-difluoro-benzyl bromide and cleavage of the BOC group by means of hydrogen chloride in methanol, (3RS,4RS)-4-[4-(3-benzyloxy-propoxy)-phenyl]-3-(2,4-difluoro-benzyloxy)-piperidine hydrochloride as a colorless oil; MS: 468 (M+H)⁺;

35)—by alkylation with 3,5-difluoro-benzyl bromide and cleavage of the BOC group by means of hydrogen chloride in methanol, (3RS,4RS)-4-[4-(3-benzyloxy-propoxy)-phenyl]-3-(3,5-difluoro-benzyloxy)-piperidine hydrochloride as a colorless oil; MS: 468 (M+H)⁺;

36)—by alkylation with methyl 4-bromomethyl-benzoate, saponification of the methyl ester during the aqueous working-up and subsequent cleavage of the BOC group by means of hydrogen chloride in methanol, 4-{(3RS,4RS)-4-[4-(3-benzyloxy-propoxy)-phenyl]-piperidin-3-yloxymethyl}-benzoic acid hydrochloride as a colorless oil; MS: 476 (M+H)⁺;

37)—by alkylation with 1-bromomethyl-4-trifluoromethoxy-benzene and cleavage of the BOC group by means of hydrogen chloride in methanol, (3RS,4RS)-4-[4-(3-benzyloxy-propoxy)-phenyl]-3-(4-trifluoromethoxy-benzyloxy)-piperidine hydrochloride as a colorless oil; MS: 516 (M+H)⁺;

38)—by alkylation with 3-bromomethyl-benzonitrile and cleavage of the BOC group by means of hydrogen chloride in methanol, 3-{(3RS,4RS)-4-[4-(3-benzyloxy-propoxy)-phenyl]-piperidin-3-yloxymethyl}-benzonitrile hydrochloride as a colorless oil; MS: 457 (M+H)⁺;

39)—by alkylation with 4-bromo-2-fluoro-benzyl bromide and cleavage of the BOC group by means of hydrogen chloride in methanol, (3RS,4RS)-4-[4-(3-benzyloxy-propoxy)-phenyl]-3-(4-bromo-2-fluoro-benzyloxy)-piperidine hydrochloride as a colorless oil; MS: 529 (M+H)⁺;

40)—by alkylation with 3-chloro-benzyl bromide and cleavage of the BOC group by means of hydrogen chloride in methanol, (3RS,4RS)-4-[4-(3-benzyloxy-propoxy)-phenyl]-3-(3-chloro-benzyloxy)-piperidine hydrochloride as a colorless oil; MS: 466 (M+H)⁺;

41)—by alkylation with 3-chloro-2-fluoro-benzyl bromide and cleavage of the BOC group by means of hydrogen chloride in methanol, (3RS,4RS)-4-[4-(3-benzyloxy-propoxy)-phenyl]-3-(3-chloro-2-fluoro-benzyloxy)-piperidine hydrochloride as a colorless oil; MS: 484 (M+H)⁺;

42)—by alkylation with 3,5-dibromo-benzyl bromide and cleavage of the BOC group by means of hydrogen chloride in methanol, (3RS,4RS)-4-[4-(3-benzyloxy-propoxy)-phenyl]-3-(3,5-dibromo-benzyloxy)-piperidine hydrochloride as a colorless oil; MS: 590 (M+H)⁺;

43)—by alkylation with 2,5-dimethoxy-benzyl chloride and cleavage of the BOC group by means of hydrogen chloride in methanol, (3RS,4RS)-4-[4-(3-benzyloxy-propoxy)-phenyl]-3-(2,5-dimethoxy-benzyloxy)-piperidine hydrochloride as a colorless oil; MS: 492 (M+H)⁺;

44)—by alkylation with 2-methy-benzyl chloride and cleavage of the BOC group by means of hydrogen chloride in methanol, (3RS,4RS)-4-[4-(3-benzyloxy-propoxy)-phenyl]-3-(2-methyl-benzyloxy)-piperidine as a colorless oil; MS: 446 (M+H)⁺;

45)—by alkylation with 3-bromomethyl-pyridine and cleavage of the BOC group by means of zinc bromide in methylene chloride, 3-[(3RS,4RS)-4-[4-(3-benzyloxy-propoxy)-phenyl]-piperidin-3-yloxymethyl]-pyridine as a colorless oil; R_{f}: 0.08 (SiO₂, methylene chloride:methanol=98:2, extracted against 5 vol. % saturated ammonia);

46)—by alkylation with 4-methylthio-benzyl chloride [J. Org. Chem. (1988), 53(3), 561-569] and cleavage of the BOC group by means of zinc bromide in methylene chloride, (3RS,4RS)-4-[4-(3-benzyloxy-propoxy)-phenyl]-3-(4-methylsulfanyl-benzyloxy)-piperidine hydrochloride as a colorless oil; MS: 478 (M+H)⁺;

47)—by alkylation with 5-chloromethyl-benzo[1.3]dioxol and cleavage of the BOC group by means of zinc bromide in methylene chloride, (3RS,4RS)-3-(benzo[1,3]dioxol-5-ylmethoxy)-4-[4-(3-benzyloxy-propoxy)-phenyl]-piperidine hydrobromide as a colorless oil; MS: 476 (M+H)⁺;

48)—by alkylation with 4-methoxy-benzyl chloride and cleavage of the BOC group by means of zinc bromide in methylene chloride, (3RS,4RS)-4-[4-(3-benzyloxy-propoxy)-phenyl]-3-(4-methoxy-benzyloxy)-piperidine as a colorless oil; MS: 462 (M+H)⁺;

49)—by alkylation with 3,4,5-trimethoxy-benzyl chloride and cleavage of the BOC group by means of zinc bromide in methylene chloride, (3RS,4RS)-4-[4-(3-benzyloxy-propoxy)-phenyl]-3-(3,4,5-trimethoxy-benzyloxy)-piperidine as a colorless oil; MS: 522 (M+H)⁺;

50)—by alkylation with 4-methoxy-3-methyl-benzyl chloride and cleavage of the BOC group by means of zinc bromide in methylene chloride, (3RS,4RS)-4-[4-(3-benzyloxy-propoxy)-phenyl]-3-(4-methoxy-3-methyl-benzyloxy)-piperidine as a colorless oil; MS: 476 (M+H)⁺;

51)—by alkylation with 3,5-dimethoxy-benzyl chloride and cleavage of the BOC group by means of zinc bromide in methylene chloride, (3RS,4RS)-4-[4-(3-benzyloxy-propoxy)-phenyl]-3-(3,5-dimethoxy-benzyloxy)-piperidine as a colorless oil; MS: 492 (M+H)⁺;

52)—by alkylation with 2,3,5,6-tetramethyl-benzyl chloride and cleavage of the BOC group by means of hydrogen chloride in methanol, (3RS,4RS)-4-[4-(3-benzyloxy-propoxy)-phenyl]-3-(2,3,5,6-tetramethyl-benzyloxy)-piperidine hydrochloride as a colorless oil; MS: 488 (M+H)⁺;

53)—by alkylation with 3-methyl-benzyl bromide and cleavage of the BOC group by means of hydrogen chloride in methanol, (3RS,4RS)-4-[4-(3-benzyloxy-propoxy)-phenyl]-3-(3-methyl-benzyloxy)-piperidine hydrochloride as a colorless oil; MS: 446 (M+H)⁺;

54)—by alkylation with 4-chloro-benzyl chloride and cleavage of the BOC group by means of hydrogen chloride in methanol, (3RS,4RS)-4-[4-(3-benzyloxy-propoxy)-phenyl]-3-(4-chloro-benzyloxy)-piperidine hydrochloride as a colorless oil; MS: 466 (M+H)⁺.

### Example 105

The following compounds were obtained analogously to Example 12 (b) by synthesizing the corresponding BOC derivatives by reacting the corresponding benzyl bromides with 3 equivalents of the respective alcoholates and using these derivatives without further purification and characterization in the cleavage of the BOC group by means of hydrogen chloride in methanol analogously to Example 22 (l) or by means of zinc bromide in methylene chloride analogously to Example 10 (b):

1)—By reaction of tert-butyl (3RS,4RS)-4-[4-(3-benzyloxy-propoxy)-phenyl]-3-(3-bromomethyl-benzyloxy)-piperidine-1-carboxylate with ethanol and cleavage of the BOC group by means of zinc bromide in methylene chloride, (3RS,4RS)-4-[4-(3-benzyloxy-propoxy)-phenyl]-3-(3-ethoxymethyl-benzyloxy)-piperidine, MS: 490 (M+H)⁺, and (3-[(3RS,4RS)-4-[4-(3-benzyloxy-propoxy)-phenyl]-piperidin-3-yloxymethyl]-phenyl]-methanol, MS: 479 (M+NH₄)⁺, each as a colorless oil;

2)—by reaction of tert-butyl (3RS,4RS)-4-[4-(3-benzyloxy-propoxy)-phenyl]-3-(3-bromomethyl-benzyloxy)-piperidine-1-carboxylate with cyclobutyl-methanol and cleavage of the BOC group by means of hydrogen chloride in methanol, (3RS,4RS)-4-[4-(3-benzyloxy-propoxy)-phenyl]-3-(3-cyclobutylmethoxymethyl-benzyloxy)-piperidine as a colorless oil; MS: 530 (M+H)⁺;

3)—by reaction of tert-butyl (3RS,4RS)-4-[4-(3-benzyloxy-propoxy)-phenyl]-3-(3-bromomethyl-benzyloxy)-piperidine-1-carboxylate with 3-phenyl-propan-1-ol and cleavage of the BOC group by means of hydrogen chloride in methanol, (3RS,4RS)-4-[4-(3-benzyloxy-propoxy)-phenyl]-3-[3-(3-phenyl-propoxymethyl)-benzyloxy]-piperidine as a colorless oil; MS: 580 (M+H)⁺;

4)—by reaction of tert-butyl (3RS,4RS)-4-[4-(3-benzyloxy-propoxy)-phenyl]-3-(3-bromomethyl-benzyloxy)-piperidine-1-carboxylate with 3,3-dimethyl-butan-1-ol and cleavage of the BOC group by means of hydrogen chloride in methanol, (3RS,4RS)-4-[4-(3-benzyloxy-propoxy)-phenyl]-3-[3-(3,3-dimethyl-butoxymethyl)-benzyloxy]-piperidine as a colorless oil; MS: 546 (M+H)⁺;

5)—by reaction of tert-butyl (3RS,4RS)-4-[4-(3-benzyloxy-propoxy)-phenyl]-3-(3-bromomethyl-benzyloxy)-piperidine-1-carboxylate with pyridin-3-yl-methanol and cleavage of the BOC group by means of hydrogen chloride in methanol, 3-[3-[(3RS,4RS)-4-[4-(3-benzyloxy-propoxy)-phenyl]-piperidin-3-yloxymethyl]-benzyloxymethyl]-pyridine as a colorless oil; MS: 553 (M+H)⁺;

6)—by reaction of tert-butyl (3RS,4RS)-4-[4-(3-benzyloxy-propoxy)-phenyl]-3-(3-bromomethyl-benzyloxy)-piperidine-1-carboxylate with pyridin-4-yl-methanol and cleavage of the BOC group by means of hydrogen chloride in methanol, 4-[3-[(3RS,4RS)-4-[4-(3-benzyloxy-propoxy)-phenyl]-piperidin-3-yloxymethyl]-benzyloxymethyl]-pyridine as a colorless oil; MS: 553 (M+H)⁺;

7)—by reaction of tert-butyl (3RS,4RS)-4-[4-(3-benzyloxy-propoxy)-phenyl]-3-(3-bromomethyl-benzyloxy)-piperidine-1-carboxylate with 2-pyridin-2-yl-ethanol and cleavage of the BOC group by means of hydrogen chloride in methanol, 2-[2-[3-[(3RS,4RS)-4-[4-(3-benzyloxy-propoxy)-phenyl]-piperidin-3-yloxymethyl]-benzyloxy]-ethyl]-pyridine as a colorless oil; MS: 567 (M+H)⁺;

8)—by reaction of tert-butyl (3RS,4RS)-4-[4-(3-benzyloxy-propoxy)-phenyl]-3-(4-bromomethyl-benzyloxy)-piperidine-1-carboxylate with cyclobutyl-methanol and cleavage of the BOC group by means of hydrogen chloride in methanol, (3RS,4RS)-4-[4-(3-benzyloxy-propoxy)-phenyl]-3-(4-cyclobutylmethoxymethyl-benzyloxy)-piperidine as a colorless oil; MS: 530 (M+H)⁺;

9)—by reaction of tert-butyl (3RS,4RS)-4-[4-(3-benzyloxy-propoxy)-phenyl]-3-(4-bromomethyl-benzyloxy)-piperidine-1-carboxylate with 3-phenyl-propan-1-ol and cleavage of the BOC group by means of hydrogen chloride in methanol, (3RS,4RS)-4-[4-(3-benzyloxy-propoxy)-phenyl]-3-[4-(3-phenyl-propoxymethyl)-benzyloxy]-piperidine as a colorless oil; MS: 580 (M+H)⁺;

10)—by reaction of tert-butyl (3RS,4RS)-4-[4-(3-benzyloxy-propoxy)-phenyl]-3-(4-bromomethyl-benzyloxy)-piperidine-1-carboxylate with 3,3-dimethyl-butan-1-ol and cleavage of the BOC group by means of hydrogen chloride in methanol, (3RS,4RS)-4-[4-(3-benzyloxy-propoxy)-phenyl]-3-[4-(3,3-dimethyl-butoxymethyl)-benzyloxy]-piperidine as a colorless oil; MS: 546 (M+H)⁺;

11)—by reaction of tert-butyl (3RS,4RS)-4-[4-(3-benzyloxy-propoxy)-phenyl]-3-(4-bromomethyl-benzyloxy)-piperidine-1-carboxylate with pyridin-3-yl-methanol and cleavage of the BOC group by means of hydrogen chloride in methanol, 3-[4-[(3RS,4RS)-4-[4-(3-benzyloxy-propoxy)-phenyl]-piperidin-3-yloxymethyl]-benzyloxymethyl]-pyridine as a colorless oil; MS: 553 (M+H)⁺;

12)—by reaction of tert-butyl (3RS,4RS)-4-[4-(3-benzyloxy-propoxy)-phenyl]-3-(4-bromomethyl-benzyloxy)-piperidine-1-carboxylate with pyridin-4-yl-methanol and cleavage of the BOC group by means of hydrogen chloride in methanol, 4-[4-[(3RS,4RS)-4-[4-(3-benzyloxy-propoxy)-phenyl]-piperidin-3-yloxymethyl]-benzyloxymethyl]-pyridine as a colorless oil; MS: 553 (M+H)⁺;

13)—by reaction of tert-butyl (3RS,4RS)-4-[4-(3-benzyloxy-propoxy)-phenyl]-3-(4-bromomethyl-benzyloxy)-piperidine-1-carboxylate with 2-pyridin-2-yl-ethanol and cleavage of the BOC group by means of hydrogen chloride in methanol, 2-[2-[4-[(3RS,4RS)-4-[4-(3-benzyloxy-propoxy)-phenyl]-piperidin-3-yloxymethyl]-benzyloxy]-ethyl]-pyridine as a colorless oil; MS: 567 (M+H)⁺;

14)—by reaction of tert-butyl (3RS,4RS)-4-[4-(3-benzyloxy-propoxy)-phenyl]-3-(2-bromomethyl-benzyloxy)-piperidine-1-carboxylate with 3-phenyl-propan-1-ol and cleavage of the BOC group by means of hydrogen chloride in methanol, (3RS,4RS)-4-[4-(3-benzyloxy-propoxy)-phenyl]-3-[2-(3-phenyl-propoxymethyl)-benzyloxy]-piperidine as a colorless oil; MS: 580 (M+H)⁺;

15)—by reaction of tert-butyl (3RS,4RS)-4-[4-(3-benzyloxy-propoxy)-phenyl]-3-(2-bromomethyl-benzyloxy)-piperidine-1-carboxylate with 3,3-dimethyl-butan-1-ol and cleavage of the BOC group by means of hydrogen chloride in methanol, (3RS,4RS)-4-[4-(3-benzyloxy-propoxy)-phenyl]-3-[2-(3,3-dimethyl-butoxymethyl)-benzyloxy]-piperidine as a colorless oil; MS: 546 (M+H)⁺;

16)—by reaction of tert-butyl (3RS,4RS)-4-[4-(3-benzyloxy-propoxy)-phenyl]-3-(2-bromomethyl-benzyloxy)-piperidine-1-carboxylate with pyridin-3-yl-methanol and cleavage of the BOC group by means of hydrogen chloride in methanol, 3-[2-[(3RS,4RS)-4-[4-(3-benzyloxy-propoxy)-phenyl]-piperidin-3-yloxymethyl]-benzyloxymethyl]-pyridine as a colorless oil; MS: 553 (M+H)⁺;

17)—by reaction of tert-butyl (3RS,4RS)-4-[4-(3-benzyloxy-propoxy)-phenyl]-3-(2-bromomethyl-benzyloxy)-piperidine-1-carboxylate with pyridin-4-yl-methanol and cleavage of the BOC group by means of hydrogen chloride in methanol, 4-[2-[(3RS,4RS)-4-[4-(3-benzyloxy-propoxy)-phenyl]-piperidin-3-yloxymethyl]-benzyloxymethyl]-pyridine as a colorless oil; MS: 553 (M+H)⁺;

18)—by reaction of tert-butyl (3RS,4RS)-4-[4-(3-benzyloxy-propoxy)-phenyl]-3-(2-bromomethyl-benzyloxy)-piperidine-1-carboxylate with 2-pyridin-2-yl-ethanol and cleavage of the BOC group by means of hydrogen chloride in methanol, 2-[2-[2-[(3RS,4RS)-4-[4-(3-benzyloxy-propoxy)-phenyl]-piperidin-3-yloxymethyl]-benzyloxy]-ethyl]-pyridine as a colorless oil; MS: 567 (M+H)⁺.

19)—by reaction of tert-butyl (3RS,4RS)-4-[4-(3-benzyloxy-propoxy)-phenyl]-3-(2-bromomethyl-benzyloxy)-piperidine-1-carboxylate with cyclobutyl-methanol and cleavage of the BOC group by means of hydrogen chloride in methanol, (3RS,4RS)-4-[4-(3-benzyloxy-propoxy)-phenyl]-3-(2-cyclobutylmethoxymethyl-benzyloxy)-piperidine as a colorless oil; MS: 530 (M+H)⁺.

The benzyl bromides used as starting materials were prepared by alkylating tert-butyl (3RS,4RS)-4-[4-(3-benzyloxy-propoxy)-phenyl]-3-hydroxy-piperidine-1-carboxylate [Example 86 (n)] with the corresponding bis-bromomethyl-benzene in an analogous manner to that described in Example 1 (g), but using 4 equivalents of dibromide and by cautious hydrolysis of the reaction solution with ice-cold sodium bicarbonate solution:

(a)—with 1,3-bis-bromomethyl-benzene, tert-butyl (3RS,4RS)-4-[4-(3-benzyloxy-propoxy)-phenyl]-3-(3-bromomethyl-benzyloxy)-piperidine-1-carboxylate as a colorless resin; MS: 624, 626 (M+H)⁺;

(b)—with 1,4-bis-bromomethyl-benzene, tert-butyl (3RS,4RS)-4-[4-(3-benzyloxy-propoxy)-phenyl]-3-(4-bromomethyl-benzyloxy)-piperidine-1-carboxylate as a yellowish oil; MS: 643, 645 (M+NH₄)⁺;

(c)—with 1,2-bis-bromomethyl-benzene, tert-butyl (3RS,4RS)-4-[4-(3-benzyloxy-propoxy)-phenyl]-3-(2-bromomethyl-benzyloxy)-piperidine-1-carboxylate as a colorless oil; MS: 624, 626 (M+H)⁺.

### Example 106

The following compounds were obtained analogously to Example 1 (g) by synthesizing the corresponding BOC derivatives by reacting the corresponding benzyl bromides with 3 equivalents of the respective alkoholates and using these derivatives, unless indicated otherwise, without further purification and characterization in the cleavage of the BOC group by means of hydrogen chloride in methanol analogously to Example 22 (l):

1)—by reaction of tert-butyl (3RS,4RS)-4-[4-(3-benzyloxy-propoxy)-phenyl]-3-(6-bromomethyl-naphthalen-2-ylmethoxy)-piperidine-1-carboxylate with (RS)-(2,2-dimethyl-[1,3]dioxolan-4-yl)-methanol and subsequent simultaneous cleavage of the BOC and dioxolan groups, a mixture of (3RS,4RS)- and (3SR,4SR)-4-[4-(3-benzyloxy-propoxy)-phenyl]-3-[6-[(RS)-2,3-dihydroxy-propoxymethyl]-naphthalen-2-ylmethoxy]-piperidine as a colorless oil; MS: 586 (M+H)⁺.

2)—by reaction of tert-butyl (3RS,4RS)-4-[4-(3-benzyloxy-propoxy)-phenyl]-3-(7-bromomethyl-naphthalen-2-ylmethoxy)-piperidine-1-carboxylate with (RS)-(2,2-dimethyl-[1,3]dioxolan-4-yl)-methanol and subsequent simultaneous cleavage of the BOC and dioxolan groups, a mixture of (3RS,4RS)- and (3SR,4SR)-4-[4-(3-benzyloxy-propoxy)-phenyl]-3-[7-[(RS)-2,3-dihydroxy-propoxymethyl]-naphthalen-2-ylmethoxy]-piperidine as a colorless oil; MS: 586 (M+H)⁺.

3)—by reaction of tert-butyl (3RS,4RS)-4-[4-(3-benzyloxy-propoxy)-phenyl]-3-(7-bromomethyl-naphthalen-2-ylmethoxy)-piperidine-1-carboxylate with rac-2-[(tetrahydro-2H-pyran-2-yl)oxy]-1-ethanol, a mixture of (3RS,4RS)- and (3SR,4SR)-4-[4-(3-benzyloxy-propoxy)-phenyl]-3-{7-[2-[(RS)-tetrahydro-pyran-2-yloxy]-ethoxymethyl]-naphthalen-2-ylmethoxy}-piperidine-1-carboxylate [colorless oil, MS:740 (M+H)⁺] and tert-butyl (3RS,4RS)-4-[4-(3-benzyloxy-propoxy)-phenyl]-3-(7-dimethylaminomethyl-naphthalen-2-ylmethoxy)-piperidine-1-carboxylate [byproduct, colorless oil, MS: 639 (M)⁺]. Simultaneous cleavage of the BOC and tetrahydropyranyl groups from the main product gave 2-(7-{(3RS,4RS)-4-[4-(3-benzyloxy-propoxy)-phenyl]-piperidin-3-yloxymethyl}-naphthalen-2-ylmethoxy)-ethanol as a colorless oil; MS: 556 (M+H)⁺. Cleavage of the BOC group from the byproduct gave (7-{(3RS,4RS)-4-[4-(3-benzyloxy-propoxy)-phenyl]-piperidin-3-yloxymethyl}-naphthalen-2-ylmethyl)-dimethyl-amine as a colorless oil; MS: 539 (M+H)⁺.

4)—by reaction of a mixture of tert-butyl (3RS,4RS)-4-[4-(3-benzyloxy-propoxy)-phenyl]-3-(8-bromomethyl-naphthalen-2-ylmethoxy)-piperidine-1-carboxylate and tert-butyl (3RS,4RS)-4-[4-(3-benzyloxy-propoxy)-phenyl]-3-(7-bromomethyl-naphthalen-1-ylmethoxy)-piperidine-1-carboxylate with (RS)-(2,2-dimethyl-[1,3]dioxolan-4-yl)-methanol and subsequent simultaneous cleavage of the BOC and dioxolan groups, a mixture of (3RS,4RS)- and (3SR,4SR)-4-[4-(3-benzyloxy-propoxy)-phenyl]-3-[8-[(RS)-2,3-dihydroxy-prop oxymethyl]-naphthalen-2-ylmethoxy]-piperidine and (3RS,4RS)- and (3SR,4SR)-4-[4-(3-benzyloxy-propoxy)-phenyl]-3-[7-[(RS)-2,3-dihydroxy-propoxymethyl]-naphthalen-1-ylmethoxy]-piperidine as a colorless oil; MS: 586 (M+H)⁺.

The naphthylmethyl bromides used as starting materials were prepared by alkylating tert-butyl (3RS,4RS)-4-[4-(3-benzyloxy-propoxy)-phenyl]-3-hydroxy-piperidine-1-carboxylate [Example 86 (n)] with the corresponding bis-bromomethyl-naphthalene in an analogous manner to that described in Example 1 (g), but using 4 equivalents of dibromide and by cautious hydrolysis of the reaction solution with ice-cold sodium bicarbonate solution:

(a)—with 2,6-bis-bromomethyl-naphthalene [J. Chem. Soc. (1961), 3741-3748], tert-butyl (3RS,4RS)-4-[4-(3-benzyloxy-propoxy)-phenyl]-3-(6-bromomethyl-naphthalen-2-ylmethoxy)-piperidine-1-carboxylate as a colorless oil; MS: 675 (M+H)⁺;

(b)—with 2,7-bis-bromomethyl-naphthalene [J. Am. Chem. Soc. (1979), 101 (15), 4259-4267], tert-butyl (3RS,4RS)-4-[4-(3-benzyloxy-propoxy)-phenyl]-3-(7-bromomethyl-naphthalen-2-ylmethoxy)-piperidine-1-carboxylate as a colorless oil; MS: 675 (M+H);

(c)—with 1,7-bis-bromomethyl-naphthalene [Chem. Ber. 91, 1981 (1958)], a mixture of tert-butyl (3RS,4RS)-4-[4-(3-benzyloxy-propoxy)-phenyl]-3-(8-bromomethyl-naphthalen-2-ylmethoxy)-piperidine-1-carboxylate and tert-butyl (3RS,4RS)-4-[4-(3-benzyloxy-propoxy)-phenyl]-3-(7-bromomethyl-naphthalen-1-ylmethoxy)-piperidine-1-carboxylate as a colorless oil; MS: 675 (M+H)⁺.

### Example 107

The following compounds were obtained analogously to Example 1 (g) by synthesizing the corresponding BOC derivatives by alkylating of tert-butyl (3RS,4RS)-4-[4-(3-hydroxy-propoxy)-phenyl]-3-(naphthalen-2-ylmethoxy)-piperidine-1-carboxylate Example 57 (c)] and using these derivatives without further purification and characterization in the cleavage of the BOC group by means of hydrogen chloride in methanol analogously to Example 22 (l):

1)—By alkylation with 4-fluoro-benzyl bromide and cleavage of the BOC group, (3RS,4RS)-4-{4-[3-(4-fluoro-benzyloxy)-propoxy]-phenyl}-3-(naphthalen-2-yl methoxy)-piperidine hydrochloride as a colorless oil; MS: 500 (M+H)⁺;

2)—by alkylation with 2-chloro-benzyl bromide and cleavage of the BOC group, (3RS,4RS)-4-{4-[3-(2-chloro-benzyloxy)-propoxy]-phenyl}-3-(naphthalen-2-yl methoxy)-piperidine as a colorless oil; MS: 516 (M)⁺;

3)—by alkylation with 2,3,4,5,6-pentafluoro-benzyl bromide and cleavage of the BOC group, (3RS,4RS)-3-(naphthalen-2-ylmethoxy)-4-{4-[3-(2,3,4,5,6-pentafluoro-benzyl oxy)-propoxy]-phenyl}-piperidine as a colorless oil; MS: 572 (M+H)⁺;

4)—by alkylation with 4-bromo-benzyl bromide and cleavage of the BOC group, (3RS,4RS)-4-{4-[3-(4-bromo-benzyloxy)-propoxy]-phenyl}-3-(naphthalen-2-ylmethoxy)-piperidine hydrochloride as a colorless oil; MS: 560 (M)⁺;

5)—by alkylation with 3-bromo-benzyl bromide and cleavage of the BOC group, (3RS,4RS)-4-{4-[3-(3-bromo-benzyloxy)-propoxy]-phenyl}-3-(naphthalen-2-ylmethoxy)-piperidine hydrochloride as a colorless oil; MS: 560 (M)⁺;

6)—by alkylation with 4-iodo-benzyl bromide and cleavage of the BOC group, (3RS,4RS)-4-{4-[3-(4-iodo-benzyloxy)-propoxy]-phenyl}-3-(naphthalen-2-ylmethoxy)-piperidine hydrochloride as a colorless oil; MS: 608 (M+H)⁺;

7)—by alkylation with 2-trifluoromethyl-benzyl bromide and cleavage of the BOC group, (3RS,4RS)-3-(naphthalen-2-ylmethoxy)-4-{4-[3-(2-trifluoromethyl-benzyloxy)-propoxy]-phenyl}-piperidine hydrochloride as a colorless oil; MS: 550 (M+H)⁺;

8)—by alkylation with 3-trifluoromethyl-benzyl bromide and cleavage of the BOC group, (3RS,4RS)-3-(naphthalen-2-ylmethoxy)-4-{4-[3-(3-trifluoromethyl-benzyloxy)-propoxy]-phenyl}-piperidine hydrochloride as a colorless oil; MS: 550 (M+H)⁺;

9)—by alkylation with 4-trifluoromethyl-benzyl bromide and cleavage of the BOC group, (3RS,4RS)-3-(naphthalen-2-ylmethoxy)-4-{4-[3-(4-trifluoromethyl-benzyloxy)-propoxy]-phenyl}-piperidine hydrochloride as a colorless oil; MS: 550 (M+H)⁺;

10)—by alkylation with 2-fluoro-benzyl bromide-benzyl bromide and cleavage of the BOC group, (3RS,4RS)-4-{4-[3-(2-fluoro-benzyloxy)-propoxy]-phenyl}-3-(naphthalen-2-ylmethoxy)-piperidine as a colorless oil; MS: 500 (M+H)⁺;

11)—by alkylation with 2-methyl-benzyl chloride and cleavage of the BOC group, (3RS,4RS)-4-{4-[3-(2-methyl-benzyloxy)-propoxy]-phenyl}-3-(naphthalen-2-yl methoxy)-piperidine hydrochloride as a colorless oil; MS: 496 (M+H)⁺;

12)—by alkylation with 3,5-dimethyl-benzyl bromide and cleavage of the BOC group, (3RS,4RS)-4-{4-[3-(3,5-dimethyl-benzyloxy)-propoxy]-phenyl}-3-(naphthalen-2-ylmethoxy)-piperidine hydrochloride as a colorless oil; MS: 510 (M+H)⁺;

13)—by alkylation with 3-methoxy-benzyl chloride and cleavage of the BOC group, (3RS,4RS)-4-{4-[3-(3-methoxy-benzyloxy)-propoxy]-phenyl}-3-(naphthalen-2-ylmethoxy)-piperidine hydrochloride as a colorless oil; MS: 512 (M+H)⁺;

14)—by alkylation with 4-isopropyl-benzyl chloride and cleavage of the BOC group, (3RS,4RS)-4-{4-[3-(4-isopropyl-benzyloxy)-propoxy]-phenyl}-3-(naphthalen-2-ylmethoxy)-piperidine hydrochloride as a colorless oil; MS: 524 (M+H)⁺;

15)—by alkylation with 2,4-dimethyl-benzyl chloride and cleavage of the BOC group, (3RS,4RS)-4-{4-[3-(2,4-dimethyl-benzyloxy)-propoxy]-phenyl}-3-(naphthalen-2-ylmethoxy)-piperidine hydrochloride as a colorless oil; MS: 510 (M+H)⁺;

16)—by alkylation with 4-methyl-benzyl bromide and cleavage of the BOC group, (3RS,4RS)-4-{4-[3-(4-methyl-benzyloxy)-propoxy]-phenyl}-3-(naphthalen-2-ylmethoxy)-piperidine hydrochloride as a colorless oil; MS: 496 (M+H)⁺;

17)—by alkylation with 4-tert-butyl-benzyl bromide and cleavage of the BOC group, (3RS,4RS)-4-{4-[3-(4-tert-butyl-benzyloxy)-propoxy]-phenyl}-3-(naphthalen-2-ylmethoxy)-piperidine hydrochloride as a colorless oil; MS: 538 (M+H)⁺;

18)—by alkylation with 2,3,5,6-tetramethyl-benzyl chloride and cleavage of the BOC group, (3RS,4RS)-3-(naphthalen-2-ylmethoxy)-4-{4-[3-(2,3,5,6-tetramethyl-benzyloxy)-propoxy]-phenyl}-piperidine hydrochloride as a colorless oil; MS: 538 (M+H)⁺;

19)—by alkylation with 3,5-dichloro-benzyl chloride and cleavage of the BOC group, (3RS,4RS)-4-{4-[3-(3,5-dichloro-benzyloxy)-propoxy]-phenyl}-3-(naphthalen-2-ylmethoxy)-piperidine hydrochloride as a colorless oil; MS: 550 (M+H)⁺;

20)—by alkylation with 2,4-dichloro-benzyl chloride and cleavage of the BOC group, (3RS,4RS)-4-{4-[3-(2,4-dichloro-benzyloxy)-propoxy]-phenyl}-3-(naphthalen-2-ylmethoxy)-piperidine hydrochloride as a colorless oil; MS: 550 (M+H)⁺;

21)—by alkylation with 2,6-dichloro-benzyl chloride and cleavage of the BOC group, (3RS,4RS)-4-{4-[3-(2,6-dichloro-benzyloxy)-propoxy]-phenyl}-3-(naphthalen-2-ylmethoxy)-piperidine hydrochloride as a colorless oil; MS: 550 (M+H)⁺;

22)—by alkylation with 2,5-dichloro-benzyl chloride and cleavage of the BOC group, (3RS,4RS)-4-{4-[3-(2,5-dichloro-benzyloxy)-propoxy]-phenyl}-3-(naphthalen-2-ylmethoxy)-piperidine hydrochloride as a colorless oil; MS: 550 (M+H)⁺;

23)—by alkylation with 2-chloro-6-fluoro-benzyl chloride and cleavage of the BOC group, (3RS,4RS)-4-{4-[3-(2-chloro-6-fluoro-benzyloxy)-propoxy]-phenyl}-3-(naphthalen-2-ylmethoxy)-piperidine hydrochloride as a colorless oil; MS: 534 (M+H)⁺;

24)—by alkylation with 2-iodo-benzyl chloride and cleavage of the BOC group, (3RS,4RS)-4-{4-[3-(2-iodoo-benzyloxy)-propoxy]-phenyl}-3-(naphthalen-2-ylmethoxy)-piperidine hydrochloride as a colorless oil; MS: 608 (M+H)⁺;

25)—by alkylation with 2-bromo-benzyl chloride and cleavage of the BOC group, (3RS,4RS)-4-{4-[3-(2-bromo-benzyloxy)-propoxy]-phenyl}-3-(naphthalen-2-ylmethoxy)-piperidine hydrochloride as a colorless oil; MS: 560 (M+H)⁺;

26)—by alkylation with 4-chloro-benzyl chloride and cleavage of the BOC group, (3RS,4RS)-4-{4-[3-(4-chloro-benzyloxy)-propoxy]-phenyl}-3-(naphthalen-2-ylmethoxy)-piperidine hydrochloride as a colorless oil; MS: 517 (M+H)⁺;

27)—by alkylation with 4-methylthio-benzyl chloride [J. Org. Chem. (1988), 53(3), 561-569] and cleavage of the BOC group, (3RS,4RS)-4-{4-[3-(4-methylsulfanyl-benzyloxy)-propoxy]-phenyl}-3-(naphthalen-2-ylmethoxy)-piperidine hydrochloride as a colorless oil; MS: 528 (M+H)⁺;

28)—by alkylation with a mixture of 3- and 4-vinyl-benzyl chloride and cleavage of the BOC group, a mixture of (3RS,4RS)-3-(naphthalen-2-ylmethoxy)-4-{4-[3-(3- and 4-vinyl-benzyloxy)-propoxy]-phenyl}-piperidine hydrochloride as a colorless oil; MS: 508 (M+H)⁺;

29)—by alkylation with 4-methoxy-benzyl chloride and cleavage of the BOC group, (3RS,4RS)-4-{4-[3-(4-methoxy-benzyloxy)-propoxy]-phenyl}-3-(naphthalen-2-ylmethoxy)-piperidine hydrochloride as a colorless oil; MS: 512 (M+H)⁺;

30)—by alkylation with 2,4-dimethoxy-benzyl chloride and cleavage of the BOC group, (3RS,4RS)-4-{4-[3-(2,4-dimethoxy-benzyloxy)-propoxy]-phenyl}-3-(naphthalen-2-ylmethoxy)-piperidine hydrochloride as a colorless oil; MS: 542 (M+H)⁺;

31)—by alkylation with 3,4,5-trimethoxy-benzyl chloride and cleavage of the BOC group, (3RS,4RS)-3-(naphthalen-2-ylmethoxy)-4-{4-[3-(3,4,5-trimethoxy-benzyloxy)-propoxy]-phenyl}-piperidine hydrochloride as a colorless oil; MS: 572 (M+H)⁺;

32)—by alkylation with 5-chloromethyl-benzo[1,3]dioxol and cleavage of the BOC group, (3RS,4RS)-4-{4-[3-(Benzo[1,3]dioxol-5-ylmethoxy)-propoxy]-phenyl}-3-(naphthalen-2-ylmethoxy)-piperidine hydrochloride as a colorless oil; MS: 526 (M+H)⁺;

33)—by alkylation with 3-chloro-4-methoxy-benzyl chloride and cleavage of the BOC group, (3RS,4RS)-4-{4-[3-(3-chloro-4-methoxy-benzyloxy)-propoxy]-phenyl}-3-(naphthalen-2-ylmethoxy)-piperidine hydrochloride as a colorless oil; MS: 546 (M+H)⁺;

34)—by alkylation with 3-methyl-benzyl bromide and cleavage of the BOC group, (3RS,4RS)-4-{4-[3-(3-methyl-benzyloxy)-propoxy]-phenyl}-3-(naphthalen-2-ylmethoxy)-piperidine hydrochloride as a colorless oil; MS: 496 (M+H)⁺;

35)—by alkylation with 3-fluoro-benzyl chloride and cleavage of the BOC group, (3RS,4RS)-4-{4-[3-(3-fluoro-benzyloxy)-propoxy]-phenyl}-3-(naphthalen-2-ylmethoxy)-piperidine hydrochloride as a colorless oil; MS: 500 (M+H)⁺;

36)—by alkylation with 2-methoxy-benzyl chloride and cleavage of the BOC group, (3RS,4RS)-4-{4-[3-(2-methoxy-benzyloxy)-propoxy]-phenyl}-3-(naphthalen-2-ylmethoxy)-piperidine as a colorless oil; MS: 512 (M+H)⁺;

37)—by alkylation with 2,5-dimethyl-benzyl chloride and cleavage of the BOC group, (3RS,4RS)-4-{4-[3-(2,5-dimethyl-benzyloxy)-propoxy]-phenyl}-3-(naphthalen-2-ylmethoxy)-piperidine hydrochloride as a colorless oil; MS: 510 (M+H)⁺;

38)—by alkylation with 4-ethyl-benzyl chloride and cleavage of the BOC group, (3RS,4RS)-4-{4-[3-(4-ethyl-benzyloxy)-propoxy]-phenyl}-3-(naphthalen-2-ylmethoxy)-piperidine hydrochloride as a colorless oil; MS: 510 (M+H)⁺;

### Example 108

The following compounds were obtained analogously to Example 44 (e) by synthesizing the corresponding BOC derivatives by alkylating tert-butyl (3RS,4RS)-4-{4-[3-(2-hydroxy-benzyloxy)-propoxy]-phenyl}-3-(naphthalen-2-ylmethoxy)-piperidine-1-carboxylate and using these derivatives without further purification and characterization in the cleavage of the BOC group by means of zinc bromide in methylene chloride analogously to Example 10 (b):

1)—by alkylation with 1-bromo-propane and cleavage of the BOC group, (3RS,4RS)-3-(naphthalen-2-ylmethoxy)-4-{4-[3-(2-propoxy-benzyloxy)-propoxy]-phenyl}-piperidine hydrobromide as a colorless oil; MS: 540 (M+H)⁺;

2)—by alkylation with 1-bromo-butane and cleavage of the BOC group, (3RS,4RS)-4-{4-[3-(2-butoxy-benzyloxy)-propoxy]-phenyl]-3-(naphthalen-2-yl methoxy)-piperidine hydrobromide as a colorless oil; MS: 554 (M+H)⁺;

3)—by alkylation with bromomethyl-cyclopropane and cleavage of the BOC group, (3RS,4RS)-4-{4-[3-(2-cyclopropylmethoxy-benzyloxy)-propoxy]-phenyl}-3-(naphthalen-2-ylmethoxy)-piperidine hydrobromide as a colorless oil; MS: 552 (M+H)⁺;

4)—by alkylation with ethyl iodoide and cleavage of the BOC group, (3RS,4RS)-4-{4-[3-(2-ethoxy-benzyloxy)-propoxy]-phenyl}-3-(naphthalen-2-yl methoxy)-piperidine hydrobromide as a colorless oil; MS: 526 (M+H)⁺;

5)—by alkylation with bromomethyl-cyclobutane and cleavage of the BOC group, (3RS,4RS)-4-{4-[3-(2-cyclobutylmethoxy-benzyloxy)-propoxy]-phenyl}-3-(naphthalen-2-ylmethoxy)-piperidine hydrobromide as a colorless oil; MS: 566 (M+H)⁺;

6)—by alkylation with isobutyl bromide and cleavage of the BOC group, (3RS,4RS)-4-{4-[3-(2-isobutoxy-benzyloxy)-propoxy]-phenyl}-3-(naphthalen-2-ylmethoxy)-piperidine hydrobromide as a colorless oil; MS: 554 (M+H)⁺;

7)—by alkylation with benzyl bromide and cleavage of the BOC group, (3RS,4RS)-4-{4-[3-(2-benzyloxy-benzyloxy)-propoxy]-phenyl}-3-(naphthalen-2-ylmethoxy)-piperidine hydrobromide as a colorless oil; MS: 588 (M+H)⁺;

8)—by alkylation with 4-bromo-1-butene and cleavage of the BOC group, (3RS,4RS)-4-{4-[3-(2-but-3-enyloxy-benzyloxy)-propoxy]-phenyl}-3-(naphthalen-2-ylmethoxy)-piperidine hydrobromide as a colorless oil; MS: 552 (M+H)⁺;

9)—by alkylation with allyl bromide and cleavage of the BOC group, (3RS,4RS)-4-{4-[3-(2-allyloxy-benzyloxy)-propoxy]-phenyl}-3-(naphthalen-2-ylmethoxy)-piperidine hydrobromide as a colorless oil; MS: 538 (M+H)⁺;

10)—by alkylation with bromo-cyclopropane and cleavage of the BOC group, (3RS,4RS)-4-{4-[3-(2-cyclopropyloxy-benzyloxy)-propoxy]-phenyl}-3-(naphthalen-2-ylmethoxy)-piperidine hydrobromide as a colorless oil; MS: 538 (M+H)⁺.

The tert-butyl (3RS,4RS)-4-{4-[3-(2-hydroxy-benzyloxy)-propoxy]-phenyl}-3-(naphthalen-2-ylmethoxy)-piperidine-1-carboxylate used as the starting material was obtained as follows:

(a) In an analogous manner to that described in Example 1 (g), by alkylating tert-butyl (3RS,4RS)-4-[4-(3-hydroxy-propoxy)-phenyl]-3-(naphthalen-2-ylmethoxy)-piperidine-1-carboxylate [Example 57 (c)] with 1-chloromethyl-(2-trimethylsilyl-ethoxymethoxy)-benzene [Example 17 (c)] there was obtained tert-butyl (3RS,4RS)-3-(naphthalen-2-ylmethoxy)-4-(4-{3-[2-(2-trimethylsilanyl-ethoxymethoxy)-benzyloxy]-propoxy}-phenyl)-piperidine-1-carboxylate as a colorless oil; MS: 746 (M+NH₄)⁺.

(b) A solution of 50 mg (0.069 mmol) of 3-(naphthalen-2-ylmethoxy)-4-(4-{3-[2-(2-trimethylsilanyl-ethoxymethoxy)-benzyloxy]-propoxy}-phenyl)-piperidine-1-carboxylate in 0.5 ml of methanol was cooled to 0° C. under argon and treated with 69 μl (0.138 mmol) of a 2N solution of hydrogen chloride in methanol. Subsequently, the mixture was left to warm to room temperature and was stirred for a further one hour. For the working-up, the reaction solution was treated with a 95:5 mixture of methylene chloride and methanol (extracted against 5 vol. % saturated ammonia) and evaporated to dryness under reduced pressure. For purification, the residue was chromatographed on silica gel using a 3:1 mixture of hexane and ethyl acetate as the eluent. There were obtained 33.1 mg (81% of theory) of tert-butyl (3RS,4RS)-4-{4-[3-(2-hydroxy-benzyloxy)-propoxy]-phenyl}-3-(naphthalen-2-ylmethoxy)-piperidine-1-carboxylate as a colorless oil; MS: 598 (M+H)⁺.

### Example 109

The following compounds were obtained by cleavage of the BOC group by means of hydrogen chloride in methanol analogously to Example 22 (l) or by means of zinc bromide in methylene chloride analogously to Example 10 (b):

1)—From tert-butyl (3R,4s,5S)-4-[4-(3-benzyloxy-propoxy)-phenyl]-3,5-bis-(naphthalen-2-ylmethoxy)-piperidine-1-carboxylate by means of hydrogen chloride in methanol, (3R,4s,5S)-4-[4-(3-benzyloxy-propoxy)-phenyl]-3,5-bis-(naphthalen-2-ylmethoxy)-piperidine hydrochloride as a colorless solid; MS: 638 (M+H)⁺;

2)—from tert-butyl (3RS,4SR,5SR)-4-[4-(3-benzyloxy-propoxy)-phenyl]-5-hydroxy-3-(naphthalen-2-ylmethoxy)-piperidine-1-carboxylate by means of hydrogen chloride in methanol, (3RS,4SR,5SR)-4-[4-(3-benzyloxy-propoxy)-phenyl]-3-(naphthalen-2-ylmethoxy)-piperidin-5-ol as a colorless oil; MS: 498 (M+H)⁺;

3)—from tert-butyl (3R,4s,5S)-4-[4-(3-benzyloxy-propoxy)-phenyl]-3,5-bis-(1,4-dimethoxy-naphthalen-2-ylmethoxy)-piperidine-1-carboxylate by means of zinc bromide in methylene chloride, (3R,4s,5S)-4-[4-(3-benzyloxy-propoxy)-phenyl]-3,5-bis-(1,4-dimethoxy-naphthalen-2-ylmethoxy)-piperidine as a colorless wax; MS: 758 (M+H)⁺;

4)—from tert-butyl (3RS,4SR,5SR)-4-[4-(3-benzyloxy-propoxy)-phenyl]-3-(1,4-dimethoxy-naphthalen-2-ylmethoxy)-5-hydroxy-piperidine-1-carboxylate by means of zinc bromide in methylene chloride, (3RS,4SR,5SR)-4-[4-(3-benzyloxy-propoxy)-phenyl]-3-(1,4-dimethoxy-naphthalen-2-ylmethoxy)-piperidin-5-ol as a colorless oil; MS: 558 (M+H)⁺;

5)—from tert-butyl (3R,4s,5S)-4-[4-(3-benzyloxy-propoxy)-phenyl]-3,5-bis-(2,4-dichloro-benzyl oxy)-piperidine-1-carboxylate by means of hydrogen chloride in methanol, (3R,4s,5S)-4-[4-(3-benzyloxy-propoxy)-phenyl]-3,5-bis-(2,4-dichloro-benzyl oxy)-piperidine hydrochloride as a colorless solid; MS: 676 (M+H)⁺;

6)—from tert-butyl (3RS,4SR,5SR)-4-[4-(3-benzyloxy-propoxy)-phenyl]-3-(2,4-dichloro-benzyloxy)-5-hydroxy-piperidine-1-carboxylate by means of hydrogen chloride in methanol, (3RS,4SR,5SR)-4-[4-(3-benzyloxy-propoxy)-phenyl]-3-(2,4-dichloro-benzyloxy)-piperidin-5-ol hydrochloride as a colorless oil; MS: 516 (M+H)⁺;

7)—from tert-butyl (3R,4s,5S)-4-[4-(3-benzyloxy-propoxy)-phenyl]-3,5-bis-(2,5-difluoro-benzyloxy)-piperidine-1-carboxylate by means of hydrogen chloride in methanol, (3R,4s,5S)-4-[4-(3-benzyloxy-propoxy)-phenyl]-3,5-bis-(2,5-difluoro-benzyloxy)-piperidine hydrochloride as a colorless oil; MS: 610 (M+H)⁺;

8)—from tert-butyl (3RS,4SR,5SR)-4-[4-(3-benzyloxy-propoxy)-phenyl]-3-(2,5-difluoro-benzyloxy)-5-hydroxy-piperidine-1-carboxylate by means of hydrogen chloride in methanol, (3RS,4SR,5SR)-4-[4-(3-benzyloxy-propoxy)-phenyl]-3-(2,5-difluoro-benzyloxy)-piperidin-5-ol hydrochloride as a colorless oil; MS: 484 (M+H)⁺;

9)—from tert-butyl (3R,4s,5S)-4-[4-(3-benzyloxy-propoxy)-phenyl]-3,5-bis-(4-carboxy-benzyloxy)-piperidine-1-carboxylate by means of hydrogen chloride in methanol, (3R,4s,5S)-4-[4-(3-benzyloxy-propoxy)-phenyl]-3,5-bis-(4-carboxy-benzyloxy)-piperidine hydrochloride as a colorless oil; R_{f}: 0.63 (SiO₂, methylene chloride:methanol=95:5, extracted against 5 vol. % saturated aqueous ammonia solution);

10)—from tert-butyl (3RS,4SR,5SR)-4-[4-(3-benzyloxy-propoxy)-phenyl]-3-(4-carboxy-benzyloxy)-5-hydroxy-piperidine-1-carboxylate by means of hydrogen chloride in methanol, 4-{4-(3RS,4SR,5SR)-[4-(3-benzyloxy-propoxy)-phenyl]-5-hydroxy-piperidin-3-yloxymethyl}-benzoic acid hydrochloride a colorless oil; R_{f}: 0.30 (SiO₂, methylene chloride:methanol=9:1, extracted against 5 vol. % saturated aqueous ammonia solution);

11)—from tert-butyl (3R,4s,5S)-4-[4-(3-benzyloxy-propoxy)-phenyl]-3,5-bis-(2,4-difluoro-benzyl oxy)-piperidine-1-carboxylate by means of hydrogen chloride in methanol, (3R,4s,5S)-4-[4-(3-benzyloxy-propoxy)-phenyl]-3,5-bis-(2,4-difluoro-benzyl oxy)-piperidine hydrochloride as a colorless oil; MS: 610 (M+H)⁺;

12)—from tert-butyl (3RS,4SR,5SR)-4-[4-(3-benzyloxy-propoxy)-phenyl]-3-(2,4-difluoro-benzyloxy)-5-hydroxy-piperidine-1-carboxylate by means of hydrogen chloride in methanol, (3RS,4SR,5SR)-4-[4-(3-benzyloxy-propoxy)-phenyl]-3-(2,4-difluoro-benzyloxy)-piperidin-5-ol hydrochloride as a colorless oil; R_{f}: 0.28 (SiO₂, methylene chloride:methanol=9:1, extracted against 5 vol. % saturated aqueous ammonia solution);

13)—from tert-butyl (3R,4s,5S)-4-[4-(3-benzyloxy-propoxy)-phenyl]-3,5-bis-(4-chloro-benzyloxy)-piperidine-1-carboxylate by means of hydrogen chloride in methanol, (3R,4s,5S)-4-[4-(3-benzyloxy-propoxy)-phenyl]-3,5-bis-(4-chloro-benzyloxy)-piperidine hydrochloride as a colorless oil; MS: 606 (M+H)⁺;

14)—from tert-butyl (3RS,4SR,5SR)-4-[4-(3-benzyloxy-propoxy)-phenyl]-3-(4-chloro-benzyloxy)-5-hydroxy-piperidine-1-carboxylate by means of hydrogen chloride in methanol, (3RS,4SR,5SR)-4-[4-(3-benzyloxy-propoxy)-phenyl]-3-(4-chloro-benzyloxy)-piperidin-5-ol hydrochloride as a colorless solid; MS: 482 (M+H)⁺;

15)—from tert-butyl (3R,4s,5S)-4-[4-(3-benzyloxy-propoxy)-phenyl]-3,5-bis-(3,4-dichloro-benzyloxy)-piperidine-1-carboxylate by means of hydrogen chloride in methanol, (3R,4s,5S)-4-[4-(3-benzyloxy-propoxy)-phenyl]-3,5-bis-(3,4-dichloro-benzyloxy)-piperidine hydrochloride as a colorless solid; MS: 676 (M+H)⁺;

16)—from tert-butyl (3RS,4SR,5SR)-4-[4-(3-benzyloxy-propoxy)-phenyl]-3-(3,4-dichloro-benzyloxy)-5-hydroxy-piperidine-1-carboxylate by means of hydrogen chloride in methanol, (3RS,4SR,5SR)-[4-(3-benzyloxy-propoxy)-phenyl]-3-(3,4-dichloro-benzyloxy)-piperidin-5-ol hydrochloride as a colorless solid; MS: 516 (M+H)⁺;

17)—from tert-butyl (3R,4s,5S)-4-[4-(3-benzyloxy-propoxy)-phenyl]-3,5-bis-(3,5-dichloro-benzyloxy)-piperidine-1-carboxylate by means of hydrogen chloride in methanol, (3R,4s,5S)-4-[4-(3-benzyloxy-propoxy)-phenyl]-3,5-bis-(3,5-dichloro-benzyl oxy)-piperidine hydrochloride as a colorless oil; MS: 676 (M+H)⁺;

18)—from tert-butyl (3RS,4SR,5SR)-4-[4-(3-benzyloxy-propoxy)-phenyl]-3-(3,5-dichloro-benzyloxy)-5-hydroxy-piperidine-1-carboxylate by means of hydrogen chloride in methanol, (3RS,4SR,5SR)-4-[4-(3-benzyloxy-propoxy)-phenyl]-3-(3,5-dichloro-benzyloxy)-piperidin-5-ol hydrochloride as a colorless oil; MS: 516 (M+H)⁺;

19)—from tert-butyl (3R,4s,5S)-4-[4-(3-benzyloxy-propoxy)-phenyl]-3,5-bis-(3-chloro-2-fluoro-benzyloxy)-piperidine-1-carboxylate by means of hydrogen chloride in methanol, (3R,4s,5S)-4-[4-(3-benzyloxy-propoxy)-phenyl]-3,5-bis-(3-chloro-2-fluoro-benzyloxy)-piperidine as a colorless oil; MS: 642 (M+H)⁺;

20)—from tert-butyl (3RS,4SR,5SR)-4-[4-(3-benzyloxy-propoxy)-phenyl]-3-(3-chloro-2-fluoro-benzyloxy)-5-hydroxy-piperidine-1-carboxylate by means of hydrogen chloride in methanol, (3RS,4SR,5SR)-4-[4-(3-benzyloxy-propoxy)-phenyl]-3-(3-chloro-2-fluoro-benzyloxy)-piperidin-5-ol hydrochloride as a colorless oil; MS: 500 (M+H)⁺;

21)—from tert-butyl (3R,4s,5S)-4-[4-(3-benzyloxy-propoxy)-phenyl]-3,5-bis-(quinolin-7-ylmethoxy)-piperidine-1-carboxylate by means of hydrogen chloride in methanol, (3R,4s,5S)-4-[4-(3-benzyloxy-propoxy)-phenyl]-3,5-bis-(quinolin-7-ylmethoxy)-piperidine hydrochloride as a colorless oil; MS: 640 (M+H)⁺;

22)—from tert-butyl (3RS,4SR,5SR)-4-[4-(3-benzyloxy-propoxy)-phenyl]-5-hydroxy-3-(quinolin-7-ylmethoxy)-piperidine-1-carboxylate by means of hydrogen chloride in methanol, (3RS,4SR,5SR)-4-[4-(3-benzyloxy-propoxy)-phenyl]-3-(quinolin-7-ylmethoxy)-piperidin-5-ol hydrochloride as a colorless oil; MS: 499 (M+H)⁺;

23)—from tert-butyl (3R,4s,5S)-4-[4-(3-benzyloxy-propoxy)-phenyl]-3,5-bis-(4-ethyl-benzyloxy)-piperidine-1-carboxylate by means of zinc bromide in methylene chloride, (3R,4s,5S)-4-[4-(3-benzyloxy-propoxy)-phenyl]-3,5-bis-(4-ethyl-benzyloxy)-piperidine as a colorless oil; MS: 594 (M+H)⁺;

24)—from tert-butyl (3RS,4SR,5SR)-4-[4-(3-benzyloxy-propoxy)-phenyl]-3-(4-ethyl-benzyloxy)-5-hydroxy-piperidine-1-carboxylate by means of zinc bromide in methylene chloride, (3RS,4SR,5SR)-4-[4-(3-benzyloxy-propoxy)-phenyl]-3-(4-ethyl-benzyloxy)-piperidin-5-ol as a colorless oil; MS: 476 (M+H)⁺;

25)—from tert-butyl (3R,4s,5S)-4-[4-(3-benzyloxy-propoxy)-phenyl]-3,5-bis-(4-vinyl-benzyloxy)-piperidine-1-carboxylate by means of zinc bromide in methylene chloride, (3R,4s,5S)-4-[4-(3-benzyloxy-propoxy)-phenyl]-3,5-bis-(4-vinyl-benzyloxy)-piperidine as a colorless oil; MS: 590 (M+H)⁺;

26)—from tert-butyl (3RS,4SR,5SR)-4-[4-(3-benzyloxy-propoxy)-phenyl]-5-hydroxy-3-(4-vinyl-benzyloxy)-piperidine-1-carboxylate by means of zinc bromide in methylene chloride, (3RS,4SR,5SR)-4-[4-(3-benzyloxy-propoxy)-phenyl]-3-(4-vinyl-benzyloxy)-piperidin-5-ol as a colorless oil; MS: 474 (M+H)⁺;

27)—from tert-butyl (3R,4s,5S)-4-[4-(3-benzyloxy-propoxy)-phenyl]-3,5-bis-(4-methoxy-benzyloxy)-piperidine-1-carboxylate by means of zinc bromide in methylene chloride, (3R,4s,5S)-4-[4-(3-benzyloxy-propoxy)-phenyl]-3,5-bis-(4-methoxy-benzyloxy)-piperidine as a colorless oil; MS: 598 (M+H)⁺;

28)—from tert-butyl (3RS,4SR,5SR)-4-[4-(3-benzyloxy-propoxy)-phenyl]-5-hydroxy-3-(4-methoxy-benzyloxy)-piperidine-1-carboxylate by means of zinc bromide in methylene chloride, (3RS,4SR,5SR)-4-[4-(3-benzyloxy-propoxy)-phenyl]-3-(4-methoxy-benzyloxy)-piperidin-5-ol as a colorless oil; MS: 478 (M+H)⁺;

29)—from tert-butyl (3R,4s,5S)-4-[4-(3-benzyloxy-propoxy)-phenyl]-3,5-bis-(3,4,5-trimethoxy-benzyloxy)-piperidine-1-carboxylate by means of zinc bromide in methylene chloride, (3R,4s,5S)-4-[4-(3-benzyloxy-propoxy)-phenyl]-3,5-bis-(3,4,5-trimethoxy-benzyloxy)-piperidine as a colorless oil; MS: 718 (M+H)⁺;

30)—from tert-butyl (3RS,4SR,5SR)-4-[4-(3-benzyloxy-propoxy)-phenyl]-5-hydroxy-3-(3,4,5-trimethoxy-benzyloxy)-piperidine-1-carboxylate by means of zinc bromide in methylene chloride, (3RS,4SR,5SR)-4-[4-(3-benzyloxy-propoxy)-phenyl]-3-(3,4,5-trimethoxy-benzyloxy)-piperidin-5-ol as a colorless oil; MS: 538 (M+H)⁺;

31)—from tert-butyl (3R,4s,5S)-4-[4-(3-benzyloxy-propoxy)-phenyl]-3,5-bis-(3,5-dimethoxy-benzyloxy)-piperidine-1-carboxylate by means of zinc bromide in methylene chloride, (3R,4s,5S)-4-[4-(3-benzyloxy-propoxy)-phenyl]-3,5-bis-(3,5-dimethoxy-benzyloxy)-piperidine as a colorless oil; MS: 658 (M+H)⁺;

32)—from tert-butyl (3RS,4SR,5SR)-4-[4-(3-benzyloxy-propoxy)-phenyl]-3-(3,5-dimethoxy-benzyloxy)-5-hydroxy-piperidine-1-carboxylate by means of zinc bromide in methylene chloride, (3RS,4SR,5SR)-4-[4-(3-benzyloxy-propoxy)-phenyl]-3-(3,5-dimethoxy-benzyloxy)-piperidin-5-ol as a colorless oil; MS: 508 (M+H)⁺;

33)—from tert-butyl (3R,4s,5S)-4-[4-(3-benzyloxy-propoxy)-phenyl]-3,5-bis-(4-trifluoromethoxy-benzyloxy)-piperidine-1-carboxylate by means of zinc bromide in methylene chloride, (3R,4s,5S)-4-[4-(3-benzyloxy-propoxy)-phenyl]-3,5-bis-(4-trifluoromethoxy-benzyloxy)-piperidine as a colorless oil; MS: 706 (M+H)⁺;

34)—from tert-butyl (3RS,4SR,5SR)-4-[4-(3-benzyloxy-propoxy)-phenyl]-5-hydroxy-3-(4-trifluoromethoxy-benzyloxy)-piperidine-1-carboxylate by means of zinc bromide in methylene chloride, (3RS,4SR,5SR)-4-[4-(3-benzyloxy-propoxy)-phenyl]-3-(4-trifluoromethoxy-benzyloxy)-piperidin-5-ol as a colorless oil; MS: 532 (M+H)⁺;

35)—from tert-butyl (3R,4s,5S)-4-[4-(3-benzyloxy-propoxy)-phenyl]-3,5-bis-(4-methylsulfanyl-benzyloxy)-piperidine-1-carboxylate by means of zinc bromide in methylene chloride, (3R,4s,5S)-4-[4-(3-benzyloxy-propoxy)-phenyl]-3,5-bis-(4-methylsulfanyl-benzyloxy)-piperidine as a colorless oil; MS: 630 (M+H)⁺;

36)—from tert-butyl (3RS,4SR,5SR)-4-[4-(3-benzyloxy-propoxy)-phenyl]-5-hydroxy-3-(4-methylsulfanyl-benzyloxy)-piperidine-1-carboxylate by means of zinc bromide in methylene chloride, (3RS,4SR,5SR)-4-[4-(3-benzyloxy-propoxy)-phenyl]-3-(4-methylsulfanyl-benzyloxy)-piperidin-5-ol as a colorless oil; MS: 494 (M+H)⁺;

37)—from tert-butyl (3R,4s,5S)-4-[4-(3-benzyloxy-propoxy)-phenyl]-3,5-bis-(4-isopropyl-benzyloxy)-piperidine-1-carboxylate by means of zinc bromide in methylene chloride, (3R,4s,5S)-4-[4-(3-benzyloxy-propoxy)-phenyl]-3,5-bis-(4-isopropyl-benzyloxy)-piperidine as a colorless oil; MS: 622 (M+H)⁺;

38)—from tert-butyl (3RS,4SR,5SR)-4-[4-(3-benzyloxy-propoxy)-phenyl]-5-hydroxy-3-(4-isopropyl-benzyloxy)-piperidine-1-carboxylate by means of zinc bromide in methylene chloride, (3RS,4SR,5SR)-4-[4-(3-benzyloxy-propoxy)-phenyl]-3-(4-isopropyl-benzyloxy)-piperidin-5-ol as a colorless oil; MS: 490 (M+H)⁺;

39)—from tert-butyl (3R,4s,5S)-4-[4-(3-benzyloxy-propoxy)-phenyl]-3,5-bis-(3-chloro-4-methoxy-benzyloxy)-piperidine-1-carboxylate by means of zinc bromide in methylene chloride, (3R,4s,5S)-4-[4-(3-benzyloxy-propoxy)-phenyl]-3,5-bis-(3-chloro-4-methoxy-benzyloxy)-piperidine as a colorless oil; MS: 666 (M+H)⁺;

40)—from tert-butyl (3RS,4SR,5SR)-4-[4-(3-benzyloxy-propoxy)-phenyl]-3-(3-chloro-4-methoxy-benzyloxy)-5-hydroxy-piperidine-1-carboxylate by means of zinc bromide in methylene chloride, (3RS,4SR,5SR)-4-[4-(3-benzyloxy-propoxy)-phenyl]-3-(3-chloro-4-methoxy-benzyloxy)-piperidin-5-ol as a colorless oil; MS: 512 (M+H)⁺;

41)—from tert-butyl (3R,4s,5S)-4-[4-(3-benzyloxy-propoxy)-phenyl]-3,5-bis-(4-methoxy-3-methyl-benzyloxy)-piperidine-1-carboxylate by means of zinc bromide in methylene chloride, (3R,4s,5S)-4-[4-(3-benzyloxy-propoxy)-phenyl]-3,5-bis-(4-methoxy-3-methyl-benzyloxy)-piperidine as a colorless oil; MS: 626 (M+H)⁺;

42)—from tert-butyl (3RS,4SR,5SR)-4-[4-(3-benzyloxy-propoxy)-phenyl]-5-hydroxy-3-(4-methoxy-3-methyl-benzyloxy)-piperidine-1-carboxylate by means of zinc bromide in methylene chloride, (3RS,4SR,5SR)-4-[4-(3-benzyloxy-propoxy)-phenyl]-3-(4-methoxy-3-methyl-benzyloxy)-piperidin-5-ol as a colorless oil; MS: 492 (M+H)⁺.

The BOC derivatives used as the starting materials were obtained as follows:

In an analogous manner to that described in Example 1 (g), by alkylating tert-butyl (3R,4S,5S)-4-[4-(3-benzyloxy-propoxy)-phenyl]-3,5-dihydroxy-piperidine-1-carboxylate using one equivalent of a benzylic halide there were obtained in about the same proportions unreacted starting material and the corresponding mono- and dialkylated BOC derivatives. This mixture was subsequently separated by chromatography:

(a)—By alkylation with 2-bromomethyl-naphthalene, tert-butyl (3R,4S,5S)-4-[4-(3-benzyloxy-propoxy)-phenyl]-3,5-bis-(naphthalen-2-ylmethoxy)-piperidine-1-carboxylate, MS: 739 (M+H)⁺, and tert-butyl (3RS,4SR,5SR)-4-[4-(3-benzyloxy-propoxy)-phenyl]-5-hydroxy-3-(naphthalen-2-ylmethoxy)-piperidine-1-carboxylate, MS: 598 (M+H)⁺, each as a colorless solid;

(b)—by alkylation with 2-chloromethyl-1,4-dimethoxy-naphthalene [J. Org. Chem. (1983), 48(19),3265-3268], tert-butyl (3R,4s,5S)-4-[4-(3-benzyloxy-propoxy)-phenyl]-3,5-bis-(1,4-dimethoxy-naphthalen-2-ylmethoxy)-piperidine-1-carboxylate, MS: 876 (M+H)⁺, as a colorless foam and tert-butyl (3RS,4SR,5SR)-4-[4-(3-benzyloxy-propoxy)-phenyl]-3-(1,4-dimethoxy-naphthalen-2-ylmethoxy)-5-hydroxy-piperidine-1-carboxylate, MS: 659 (M+H)⁺, as a colorless oil;

(c)—by alkylation with 2,4-dichloro-benzyl chloride, tert-butyl (3R,4s,5S)-4-[4-(3-benzyloxy-propoxy)-phenyl]-3,5-bis-(2,4-dichloro-benzyloxy)-piperidine-1-carboxylate, R_{f}: 0.83 (SiO₂, methylene chloride:ethyl acetate=8:2), and tert-butyl (3RS,4SR,5SR)-4-[4-(3-benzyloxy-propoxy)-phenyl]-3-(2,4-dichloro-benzyloxy)-5-hydroxy-piperidine-1-carboxylate, R_{f}: 0.30 (SiO₂, methylene chloride:ethyl acetate=8:2), each as a colorless oil;

(d)—by alkylation with 2,5-difluoro-benzyl bromide, tert-butyl (3R,4s,5S)-4-[4-(3-benzyloxy-propoxy)-phenyl]-3,5-bis-(2,5-difluoro-benzyl oxy)-piperidine-1-carboxylate, MS: 727 (M+NH₄)⁺, and tert-butyl (3RS,4SR,5SR)-4-[4-(3-benzyloxy-propoxy)-phenyl]-3-(2,5-difluoro-benzyloxy)-5-hydroxy-piperidine-1-carboxylate, R_{f}: 0.26 (SiO₂, methylene chloride:ethyl acetate=8:2), each as a colorless oil;

(e)—by alkylation with methyl 4-bromomethyl-benzoate and saponification of the methyl ester during the aqueous working-up, tert-butyl (3R,4s,5S)-4-[4-(3-benzyloxy-propoxy)-phenyl]-3,5-bis-(4-carboxy-benzyloxy)-piperidine-1-carboxylate, R_{f}: 0.18 (SiO₂, methylene chloride:methanol=9:1), and tert-butyl (3RS,4SR,5SR)-4-[4-(3-benzyloxy-propoxy)-phenyl]-3-(4-carboxy-benzyloxy)-5-hydroxy-piperidine-1-carboxylate, R_{f}: 0.42 (SiO₂, methylene chloride:methanol=9:1), each as a colorless oil;

(f)—by alkylation with 2,4-difluoro-benzyl bromide, tert-butyl (3R,4s,5S)-4-[4-(3-benzyloxy-propoxy)-phenyl]-3,5-bis-(2,4-difluoro-benzyl oxy)-piperidine-1-carboxylate, MS: 727 (M+NH₄)⁺, and tert-butyl (3RS,4SR,5SR)-4-[4-(3-benzyloxy-propoxy)-phenyl]-3-(2,4-difluoro-benzyloxy)-5-hydroxy-piperidine-1-carboxylate, R_{f}: 0.24 (SiO₂, methylene chloride:ethyl acetate=8:2), each as a colorless oil;

(g)—by alkylation with 4-chloro-benzyl chloride, tert-butyl (3R,4s,5S)-4-[4-(3-benzyloxy-propoxy)-phenyl]-3,5-bis-(4-chloro-benzyloxy)-piperidine-1-carboxylate, MS: 724 (M+NH₄)⁺, and tert-butyl (3RS,4SR,5SR)-4-[4-(3-benzyloxy-propoxy)-phenyl]-3-(4-chloro-benzyloxy)-5-hydroxy-piperidine-1-carboxylate, MS: 582 (M+H)⁺, each as a colorless oil;

(h)—by alkylation with 3,4-dichloro-benzyl choride, tert-butyl (3R,4s,5S)-4-[4-(3-benzyloxy-propoxy)-phenyl]-3,5-bis-(3,4-dichloro-benzyloxy)-piperidine-1-carboxylate, MS: 793 (M+NH₄)⁺, and tert-butyl (3RS,4SR,5SR)-4-[4-(3-benzyloxy-propoxy)-phenyl]-3-(3,4-dichloro-benzyloxy)-5-hydroxy-piperidine-1-carboxylate, R_{f}: 0.55 (SiO₂, methylene chloride:ethyl acetate=8:2), each as a colorless oil;

(i)—by alkylation with 3,5-dichloro-benzyl choride, tert-butyl (3R,4s,5S)-4-[4-(3-benzyloxy-propoxy)-phenyl]-3,5-bis-(3,5-dichloro-benzyl oxy)-piperidine-1-carboxylate, MS: 793 (M+NH₄)⁺, and tert-butyl (3RS,4SR,5SR)-4-[4-(3-benzyloxy-propoxy)-phenyl]-3-(3,5-dichloro-benzyloxy)-5-hydroxy-piperidine-1-carboxylate, MS: 634 (M+NH₄)⁺, each as a colorless oil;

(j)—by alkylation with 3-chloro-2-fluoro-benzyl bromide, tert-butyl (3R,4s,5S)-4-[4-(3-benzyloxy-propoxy)-phenyl]-3,5-bis-(3-chloro-2-fluoro-benzyloxy)-piperidine-1-carboxylate, MS: 760 (M+NH₄)⁺, and tert-butyl (3RS,4SR,5SR)-4-[4-(3-benzyloxy-propoxy)-phenyl]-3-(3-chloro-2-fluoro-benzyloxy)-5-hydroxy-piperidine-1-carboxylate, R_{f}: 0.54 (SiO₂, methylene chloride:ethyl acetate=8:2), each as a colorless oil;

(k)—by alkylation with 7-bromomethyl-quinoline hydrobromide [J. Am. Chem. Soc. 77, 1054 (1955)] using appropriately more sodium hydride, tert-butyl (3R,4s,5S)-4-[4-(3-benzyloxy-propoxy)-phenyl]-3,5-bis-(quinolin-7-ylmethoxy)-piperidine-1-carboxylate, MS: 740 (M+H)⁺, and tert-butyl (3RS,4SR,5SR)-4-[4-(3-benzyloxy-propoxy)-phenyl]-5-hydroxy-3-(quinolin-7-ylmethoxy)-piperidine-1-carboxylate, MS: 599 (M+H)⁺, R_{f}: 0.35 (SiO₂, methylene chloride:ethyl acetate=2:3), each as a colorless oil;

(l)—by alkylation with 4-ethyl-benzyl chloride, tert-butyl (3R,4s,5S)-4-[4-(3-benzyloxy-propoxy)-phenyl]-3,5-bis-(4-ethyl-benzyloxy)-piperidine-1-carboxylate, MS: 711 (M+NH₄)⁺, and tert-butyl (3RS,4SR,5SR)-4-[4-(3-benzyloxy-propoxy)-phenyl]-3-(4-ethyl-benzyloxy)-5-hydroxy-piperidine-1-carboxylate, R_{f}: 0.30 (SiO₂, methylene chloride:ethyl acetate=8:2), each as a colorless oil;

(m)—by alkylation with 4-vinyl-benzyl chloride, tert-butyl (3R,4s,5S)-4-[4-(3-benzyloxy-propoxy)-phenyl]-3,5-bis-(4-vinyl-benzyloxy)-piperidine-1-carboxylate, MS: 707 (M+NH₄)⁺, and tert-butyl (3RS,4SR,5SR)-4-[4-(3-benzyloxy-propoxy)-phenyl]-5-hydroxy-3-(4-vinyl-benzyloxy)-piperidine-1-carboxylate, R_{f}: 0.30 (SiO₂, methylene chloride:ethyl acetate=8:2), each as a colorless oil;

(n)—by alkylation with 4-methoxy-benzyl chloride, tert-butyl (3R,4s,5S)-4-[4-(3-benzyloxy-propoxy)-phenyl]-3,5-bis-(4-methoxy-benzyloxy)-piperidine-1-carboxylate, MS: 715 (M+NH₄)⁺, and tert-butyl (3RS,4SR,5SR)-4-[4-(3-benzyloxy-propoxy)-phenyl]-5-hydroxy-3-(4-methoxy-benzyloxy)-piperidine-1-carboxylate, MS: 595 (M+NH₄)⁺, each as a colorless oil;

(o)—by alkylation with 3,4,5-trimethoxy-benzyl chloride, tert-butyl (3R,4s,5S)-4-[4-(3-benzyloxy-propoxy)-phenyl]-3,5-bis-(3,4,5-trimethoxy-benzyloxy)-piperidine-1-carboxylate, MS: 835 (M+NH₄)⁺, and tert-butyl (3RS,4SR,5SR)-4-[4-(3-benzyloxy-propoxy)-phenyl]-5-hydroxy-3-(3,4,5-trimethoxy-benzyloxy)-piperidine-1-carboxylate, MS: 655 (M+NH₄)⁺, each as a colorless oil;

(p)—by alkylation with 3,5-dimethoxy-benzyl chloride, tert-butyl (3R,4s,5S)-4-[4-(3-benzyloxy-propoxy)-phenyl]-3,5-bis-(3,5-dimethoxy-benzyloxy)-piperidine-1-carboxylate, MS: 775 (M+NH₄)⁺, and tert-butyl (3RS,4SR,5SR)-4-[4-(3-benzyloxy-propoxy)-phenyl]-3-(3,5-dimethoxy-benzyloxy)-5-hydroxy-piperidine-1-carboxylate, MS: 625 (M+NH₄)⁺, each as a colorless oil;

(q)—by alkylation with 4-trifluoromethoxy-benzyl bromide, tert-butyl (3R,4s,5S)-4-[4-(3-benzyloxy-propoxy)-phenyl]-3,5-bis-(4-trifluoromethoxy-benzyloxy)-piperidine-1-carboxylate, MS: 823 (M+NH₄)⁺, and tert-butyl (3RS,4SR,5SR)-4-[4-(3-benzyloxy-propoxy)-phenyl]-5-hydroxy-3-(4-trifluoromethoxy-benzyloxy)-piperidine-1-carboxylate, R_{f}: 0.32 (SiO₂, methylene chloride:ethyl acetate=8:2), each as a colorless oil;

(r)—by alkylation with 4-methylthio-benzyl chloride [J. Org. Chem. (1988), 53(3), 561-569], tert-butyl (3R,4s,5S)-4-[4-(3-benzyloxy-propoxy)-phenyl]-3,5-bis-(4-methylsulfanyl-benzyloxy)-piperidine-1-carboxylate, MS: 747 (M+NH₄)⁺, and tert-butyl (3RS,4SR,5SR)-4-[4-(3-benzyloxy-propoxy)-phenyl]-5-hydroxy-3-(4-methylsulfanyl-benzyloxy)-piperidine-1-carboxylate, MS: 611 (M+NH₄)⁺, each as a colorless oil;

(s)—by alkylation with 4-isopropyl-benzyl chloride, tert-butyl (3R,4s,5S)-4-[4-(3-benzyloxy-propoxy)-phenyl]-3,5-bis-(4-isopropyl-benzyloxy)-piperidine-1-carboxylate, MS: 739 (M+NH₄)⁺, and tert-butyl (3RS,4SR,5SR)-4-[4-(3-benzyloxy-propoxy)-phenyl]-5-hydroxy-3-(4-isopropyl-benzyloxy)-piperidine-1-carboxylate, MS: 607 (M+NH₄)⁺, each as a colorless oil;

(t)—by alkylation with 3-chloro-4-methoxy-benzyl chloride, tert-butyl (3R,4s,5S)-4-[4-(3-benzyloxy-propoxy)-phenyl]-3,5-bis-(3-chloro-4-methoxy-benzyloxy)-piperidine-1-carboxylate, MS: 784 (M+NH₄)⁺, and tert-butyl (3RS,4SR,5SR)-4-[4-(3-benzyloxy-propoxy)-phenyl]-3-(3-chloro-4-methoxy-benzyloxy)-5-hydroxy-piperidine-1-carboxylate, MS: 630 (M+NH₄)⁺, each as a colorless oil;

(u)—by alkylation with 4-methoxy-3-methyl-benzyl chloride, tert-butyl (3R,4s,5S)-4-[4-(3-benzyloxy-propoxy)-phenyl]-3,5-bis-(4-methoxy-3-methyl-benzyloxy)-piperidine-1-carboxylate, MS: 743 (M+NH₄)⁺, and tert-butyl (3RS,4SR,5SR)-4-[4-(3-benzyloxy-propoxy)-phenyl]-5-hydroxy-3-(4-methoxy-3-methyl-benzyloxy)-piperidine-1-carboxylate, MS: 609 (M+NH₄)⁺, each as a colorless oil.

The tert-butyl (3R,4s,5S)-4-[4-(3-benzyloxy-propoxy)-phenyl]-3,5-dihydroxy-piperidine-1-carboxylate used as the starting material was obtained as follows:

(α) 50.0 g (179 mmol) of 1-benzyl-4-(4-methoxy-phenyl)-1,2,3,6-tetrahydro-pyridine [Example 44 (b)] were dissolved in 500 ml of tetrahydrofuran, treated at room temperature with 36.3 ml (322 mmol) of 48 percent aqueous hydrobromic acid and the reaction mixture was subsequently concentrated on a rotary evaporator. The thus-formed residue was suspended twice with 500 ml of toluene and again concentrated, then dissolved in 1500 ml of dioxan and 1200 ml of water, treated with 51.6 g (501 mmol) of sodium bromide and 9.3 ml (181 mmol) of bromine and stirred at room temperature for 2 hours. The thus-obtained orange colored solution was subsequently cooled to 0° C. and treated at 5° to 10° C. with 1240 ml of 2N sodium hydroxide solution and stirred at room temperature for a further 2 hours. Thereupon, the reaction mixture was extracted three times with 2 liters of ethyl acetate, the combined organic phases were washed with water, dried over magnesium sulfate and evaporated on a rotary evaporator at a maximum 40° C. There were thus obtained 53.64 g (about 100% of theory) of (1RS,6RS)-3-benzyl-6-(4-methoxy-phenyl)-7-oxa-3-aza-bicyclo[4.1.0]heptane in the form of a brown solid; MS: 295 (M)⁺.

(β) 53.44 g (179 mmol) of (1RS,6RS)-3-benzyl-6-(4-methoxy-phenyl)-7-oxa-3-aza-bicyclo[4.1.0]heptane were suspended in 980 ml of ether and this suspension was added dropwise under argon and with the exclusion of moisture to 226 ml of 1.6M methyllithium solution in diethyl ether (362 mmol) at room temperature. Subsequently, the reaction mixture was heated under reflux for one hour. After cooling to room temperature it was poured into 1.5 liters of saturated sodium hydrogen carbonate solution and extracted twice with 1.5 liters of ethyl acetate, the combined ethyl acetate phases were washed with water, dried over sodium sulfate and evaporated on a rotary evaporator at a maximum 40° C. There were thus obtained 52.8 g (about 100% of theory) of RS)-1-benzyl-4-(4-methoxy-phenyl)-1,2,3,6-tetrahydro-pyridin-3-ol in the form of a brown oil; MS: 296 (M+H)⁺.

(γ) 52.6 g (178 mmol) of RS)-1-benzyl-4-(4-methoxy-phenyl)-1,2,3,6-tetrahydro-pyridin-3-ol were dissolved in 300 ml of N,N-dimethylformamide, treated portionwise with 25 g (about 600 mmol) of sodium hydride dispersion in refined oil (55-65%) and the reaction mixture was heated to 50° C. under argon for 1 hour. After cooling to 5° C. the mixture was treated slowly with 23 ml (285 mmol) of ethyl iodide and stirred without cooling for one hour. Thereupon, the reaction mixture was poured into 2 liters of ice-water and extracted three times with 1 liter of ethyl acetate. The combined ethyl acetate phases were subsequently washed with water, dried over magnesium sulfate and evaporated on a rotary evaporator at a maximum 4020 C. The residue which was thereby obtained was chromatographed on silica gel with hexane/ethyl acetate (5:1). There were thus obtained 42.51 g (74% of theory) of (RS)-1-benzyl-3-ethoxy-4-(4-methoxy-phenyl)-1,2,3,6-tetrahydro-pyridine in the form of an orange colored oil; MS: 324 (M)⁺.

(δ) 42.3 g (131 mmol) of (RS)-1-benzyl-3-ethoxy-4-(4-methoxy-phenyl)-1,2,3,6-tetrahydro-pyridine were dissolved in 500 ml of 1,2-dimethoxyethane, treated with 7.45 g (196 mmol) of sodium borohydride and treated while cooling at a maximum 28° C. with a solution of 44.3 ml (353 mmol) of boron trifluoride etherate in 44.3 ml of 1,2-dimethoxyethane and the reaction mixture was stirred at room temperature for 2 hours. Subsequently, while cooling at a maximum of 35° C., 169 ml of 4.1N potassium hydroxide solution followed by 33.9 ml of 30% hydrogen peroxide solution were added dropwise and the reaction mixture was heated under reflux for 3 hours. After cooling to room temperature the reaction solution was poured into 2 liters of water and extracted twice with 1 liter of methylene chloride each time. The combined methylene chloride phases were washed with water, dried over magnesium sulfate and evaporated on a rotary evaporator at a maximum 40° C. The residue which was thereby obtained was chromatographed on silica gel with hexane/ethyl acetate (initially 4:1, then ethyl acetate content increased stepwise to 1:1). There were thus obtained 22.1 g (49% of theory) of (3RS,4RS,5SR)-5-ethoxy-1-benzyl-3-hydroxy-4-(4-methoxy-phenyl)-piperidine in the form of a yellowish oil; MS: 342 (M+H)⁺.

(ε) 52.39 g (153.4 mmol) of (3RS,4RS,5SR)-5-ethoxy-1-benzyl-3-hydroxy-4-(4-methoxy-phenyl)-piperidine were dissolved in 525 ml of methylene chloride and treated at a maximum of 40° C. with 306 ml of 1M borontribromide solution in methylene chloride and the reaction mixture was stirred at room temperature for 4 hours. Thereupon, the reaction mixture was cooled to 5° C. and the crystals formed were filtered off. These were subsequently dissolved in methylene chloride/methanol (8:2, extracted against 5 vol. % conc. aqueous ammonia) and chromatographed on silica gel with the same eluent. There were thus obtained 34.18 g (74% of theory) of (3R,4s,5S)-1-benzyl-4-(4-hydroxy-phenyl)-piperidin-3,5-diol in form of an amorphous, colorless solid; MS: 300 (M+H)⁺.

(ζ) 33.98 g (113.5 mmol) of (3R,4s,5S)-1-benzyl-4-(4-hydroxy-phenyl)-piperidin-3,5-diol were dissolved in 1.7 liters of methanol, treated with 5.1 g of palladium-on-charcoal (10%) and exhaustively hydrogenated at room temperature under normal pressure. Subsequently, the reaction mixture was filtered over silica gel and concentrated in a water-jet vacuum. There were thus obtained 22.56 g (95% of theory) of (3R,4s,5S)-4-(4-hydroxy-phenyl)-piperidine-3,5-diol in the form of an amorphous, colorless solid; MS: 209 (M)⁺.

(η) 22.36 g (106 mmol) of (3R,4s,5S)-4-(4-hydroxy-phenyl)-piperidine-3,5-diol were dissolved in 559 ml of dioxan and 186 ml of water, treated with 18.85 g (224 mmol) of sodium hydrogen carbonate and 25.65 g (117.5 mmol) of di-tert-butyl dicarbonate and stirred at room temperature for 2 hours. Thereupon, the reaction mixture was poured on to 1.5 liters of ice-water and extracted twice with 1.5 liters of ethyl acetate. The combined ethyl acetate phases were dried over magnesium sulfate and evaporated on a rotary evaporator at a maximum of 50° C. The residue thus obtained was chromatographed on silica gel with methylene chloride/methanol (95:5). There were thus obtained 15.79 g (48% of theory) of tert-butyl (3R,4s,5S)-3,5-dihydroxy-4-(4-hydroxy-phenyl)-piperidine-1-carboxylate in the form of colorless crystals, MS: 310 (M+H)⁺.

(θ) 15.59 g (50.4 mmol) of tert-butyl (3R,4s,5S)-3,5-dihydroxy-4-(4-hydroxy-phenyl)-piperidine-1-carboxylate were dissolved in 510 ml of methyl ethyl ketone, treated with 28 g (201 mmol) of potassium carbonate followed by 34.7 g (151 mmol) of benzyl-3-bromopropyl ether and the reaction mixture was heated under reflux for 24 hours. After cooling to room temperature the mixture was poured on to 800 ml of ice-water and extracted twice with 500 ml of ethyl acetate, the combined ethyl acetate phases were washed with water, dried over magnesium sulfate and evaporated on a rotary evaporator at a maximum of 40° C. The thus-obtained residue was chromatographed on silica gel with methylene chloride/ethyl acetate (7:3). There were thus obtained 20.5 g (89% of theory) of tert-butyl (3R,4s,5S)-4-[4-(3-benzyloxy-propoxy)-phenyl]-3,5-dihydroxy-piperidine-1-carboxylate in the form of an amorphous, colorless solid; MS: 458 (M+H)⁺.

### Example 110

The following compounds were obtained by cleavage of the BOC group by means of hydrogen chloride in methanol analogously to Example 22 (l) or by means of zinc bromide in methylene chloride analogously to Example 10 (b):

1)—From tert-butyl (3RS,4SR,5SR)-4-[4-(3-benzyloxy-propoxy)-phenyl]-5-ethoxy-3-(naphthalen-2-ylmethoxy)-piperidine-1-carboxylate by means of zinc bromide in methylene chloride, (3RS,4SR,5SR)-4-[4-(3-benzyloxy-propoxy)-phenyl]-5-ethoxy-3-(naphthalen-2-ylmethoxy)-piperidine as a colorless oil; MS: 526 (M+H)⁺;

2)—from tert-butyl (3RS,4RS,5SR)-4-[4-(3-benzyloxy-propoxy)-phenyl]-5-[2-(4-methyl-piperazin-1-yl)-ethoxy-3-(naphthalen-2-ylmethoxy)-piperidine-1-carboxylate by means of hydrogen chloride in methanol, 1-{2-[(3RS,4RS,5SR)-4-[4-(3-benzyloxy-propoxy)-phenyl]-3-(naphthalen-2-ylmethoxy)-piperidin-5-yloxy]-ethyl}-4-methyl-piperazine as a colorless oil; MS: 624 (M+H)⁺;

3)—from tert-butyl (3RS,4SR,5SR)-4-[4-(3-benzyloxy-propoxy)-phenyl]-3-(naphthalen-2-ylmethoxy)-5-propoxy-piperidine-1-carboxylate by means of hydrogen chloride in methanol, (3RS,4SR,5SR)-4-[4-(3-benzyloxy-propoxy)-phenyl]-3-(naphthalen-2-ylmethoxy)-5-propoxy-piperidine as a colorless oil; MS: 540 (M+H)⁺;

4)—from tert-butyl (3RS,4SR,5SR)-4-[4-(3-benzyloxy-propoxy)-phenyl]-5-butoxy-3-(naphthalen-2-ylmethoxy)-piperidine-1-carboxylate by means of hydrogen chloride in methanol, (3RS,4SR,5SR)-4-[4-(3-benzyloxy-propoxy)-phenyl]-5-butoxy-3-(naphthalen-2-ylmethoxy)-piperidine as a colorless oil; MS: 554 (M+H)⁺;

5)—from tert-butyl (3RS,4SR,5SR)-4-[4-(3-benzyloxy-propoxy)-phenyl]-5-methoxy-3-(naphthalen-2-ylmethoxy)-piperidine-1-carboxylate by means of hydrogen chloride in methanol, (3RS,4SR,5SR)-4-[4-(3-benzyloxy-propoxy)-phenyl]-5-methoxy-3-(naphthalen-2-ylmethoxy)-piperidine as a colorless oil; MS: 512 (M+H)⁺;

6)—from a mixture of tert-butyl (3RS,4RS,5SR)- and (3SR,4SR,5RS)-4-[4-(3-benzyloxy-propoxy)-phenyl]-3-(naphthalen-2-ylmethoxy)-5-[2-[(RS)-tetrahydro-pyran-2-yloxy]-ethoxy]-piperidine-1-carboxylate by means of hydrogen chloride in methanol with simultaneous cleavage of the tetrahydropyranyl group, 2-[(3RS,4RS,5SR)-4-[4-(3-benzyloxy-propoxy)-phenyl]-3-(naphthalen-2-ylmethoxy)-piperidin-5-yloxy]-ethanol as a colorless oil; MS: 542 (M+H)⁺;

7)—from a mixture of tert-butyl (3RS,4SR,5SR)- and (3SR,4RS,5RS)-4-[4-(3-benzyloxy-propoxy)-phenyl]-3-(naphthalen-2-ylmethoxy)-5-[3-[(RS)-tetrahydro-pyran-2-yloxy]-propoxy]-s piperidine-1-carboxylate by means of hydrogen chloride in methanol with simultaneous cleavage of the tetrahydropyranyl group, 3-[(3RS,4SR,5SR)-4-[4-(3-benzyloxy-propoxy)-phenyl]-3-(naphthalen-2-ylmethoxy)-piperidin-5-yloxy]-propan-1-ol as a colorless oil; MS: 556 (M+H)⁺;

8)—from a mixture of tert-butyl (3RS,4SR,5SR)- and (3SR,4RS,5RS)-4-[4-(3-benzyloxy-propoxy)-phenyl]-3-(naphthalen-2-ylmethoxy)-5-[4-((RS)-tetrahydro-pyran-2-yloxy]-butoxy]-piperidine-1-carboxylate by means of hydrogen chloride in methanol with simultaneous cleavage of the tetrahydropyranyl group, 4-[(3RS,4SR,5SR)-4-[4-(3-benzyloxy-propoxy)-phenyl]-3-(naphthalen-2-ylmethoxy)-piperidin-5-yloxy]-butan-1-ol as a colorless oil; MS: 570 (M+H)⁺.

9)—from tert-butyl (3RS,4RS,5SR)-4-[4-(3-benzyloxy-propoxy)-phenyl]-5-(2-morpholin-4-yl-ethoxy)-3-(naphthalen-2-ylmethoxy)-piperidine-1-carboxylate by means of hydrogen chloride in methanol, 4-{2-[(3RS,4RS,5SR)-4-[4-(3-benzyloxy-propoxy)-phenyl]-3-(naphthalen-2-ylmethoxy)-piperidin-5-yloxy]-ethyl}-morpholine as a colorless oil; MS: 611 (M+H)⁺;

10)—from tert-butyl (3RS,4RS)-3-(naphthalen-2-ylmethoxy)-4-(4-{3-[2-(2-trimethylsilanyl-ethoxy methoxy)-benzyloxy]-propoxy}-phenyl)-piperidine-1-carboxylate [Example 108 (b)] by means of hydrogen chloride in methanol with simultaneous cleavage of the 2-trimethylsilanyl-ethoxymethoxy group, 2-(3-{4-[(3RS,4RS)-3-(naphthalen-2-ylmethoxy)-piperidin-4-yl]-phenoxy}-propoxymethyl)-phenol as a colorless oil; MS: 579 (M+H)⁺.

The BOC derivatives used as starting materials were obtained from tert-butyl (3RS,4SR,5SR)-4-[4-(3-benzyloxy-propoxy)-phenyl]-5-hydroxy-3-(naphthalen-2-ylmethoxy)-piperidine-1-carboxylate [Example 109 (a)] as follows in an analogous manner to that described in Example 1 (g), but at a reaction temperature of 50° C. and using a large excess of sodium hydride and alkylating agent:

(a)—By alkylation with ethyl bromide, tert-butyl (3RS,4SR,5SR)-4-[4-(3-benzyloxy-propoxy)-phenyl]-5-ethoxy-3-(naphthalen-2-ylmethoxy)-piperidine-1-carboxylate as a colorless oil; MS: 626 (M+H)⁺;

(b)—by alkylation with 1-(2-chloroethyl)-4-methyl-piperazine [Austr. J. Chem. 9 (1956), 89], tert-butyl (3RS,4RS,5SR)-4-[4-(3-benzyloxy-propoxy)-phenyl]-5-[2-(4-methyl-piperazin-1-yl)-ethoxy]-3-(naphthalen-2-ylmethoxy)-piperidine-1-carboxylate as a colorless oil; MS: 724 (M+H)⁺;

(c)—by alkylation with n-propyl bromide, tert-butyl (3RS,4SR,5SR)-4-[4-(3-benzyloxy-propoxy)-phenyl]-3-(naphthalen-2-ylmethoxy)-5-propoxy-piperidine-1-carboxylate as a yellowish oil; MS: 640 (M+H)⁺;

(d)—by alkylation with n-butyl bromide, tert-butyl (3RS,4SR,5SR)-4-[4-(3-benzyloxy-propoxy)-phenyl]-5-butoxy-3-(naphthalen-2-ylmethoxy)-piperidine-1-carboxylate as a yellowish oil; MS: 654 (M+H)⁺;

(e)—by alkylation with methyl iodide, tert-butyl (3RS,4SR,5SR)-4-[4-(3-benzyloxy-propoxy)-phenyl]-5-methoxy-3-(naphthalen-2-ylmethoxy)-piperidine-1-carboxylate as a yellowish oil; MS: 612 (M+H)⁺;

(f)—by alkylation with rac-2-(2-bromoethoxy)-tetrahydropyran [J. Amer. Chem. Soc. 70, 41 87 (1948)], a mixture of tert-butyl (3RS,4RS,5SR)- and (3SR,4SR,5RS)-4-[4-(3-benzyloxy-propoxy)-phenyl]-3-(naphthalen-2-ylmethoxy)-5-[2-[(RS)-tetrahydro-pyran-2-yloxy]-ethoxy]-piperidine-1-carboxylate as a colorless oil; MS: 743 (M+NH₄)⁺.

(g)—by alkylation with rac-2-(3-bromo-propoxy)-tetrahydropyran [J. Chem. Soc. 1955, 1770], a mixture of tert-butyl (3RS,4SR,5SR)- and (3SR,4RS,5RS)-4-[4-(3-benzyloxy-propoxy)-phenyl]-3-(naphthalen-2-ylmethoxy)-5-[3-[(RS)-tetrahydro-pyran-2-yloxy]-propoxy]-piperidine-1-carboxylate as a colorless oil; MS: 740 (M+H)⁺.

(h)—by alkylation with rac-2-(4-bromo-butoxy)-tetrahydropyran [S. W. Baldwin et al., J. Org. Chem. 1985, 50, 4432-4439], a mixture of tert-butyl (3RS,4SR,5SR)- and (3SR,4RS,5RS)-4-[4-(3-benzyloxy-propoxy)-phenyl]-3-(naphthalen-2-ylmethoxy)-5-[4-[(RS)-tetrahydro-pyran-2-yloxy]-butoxy]-piperidine-1-carboxylate as a colorless oil; MS: 771 (M+NH₄)⁺.

(i)—by alkylation with 4-(2-chloroethyl)-morpholine, tert-butyl (3RS,4RS,5SR)-4-[4-(3-benzyloxy-propoxy)-phenyl]-5-(2-morpholin-4-yl-ethoxy)-3-(naphthalen-2-ylmethoxy)-piperidine-1-carboxylate as a colorless oil; MS: 711 (M+H)⁺.

### Example 111

The following compounds were obtained by cleavage of the BOC group and simultaneously of the tetrahydropyranyl protecting group (where present) by means of hydrogen chloride in methanol analogously to Example 22 (l) or of the BOC group by means of zinc bromide in methylene chloride analogously to Example 10 (b):

1)—From tert-butyl (3RS,4SR,5RS)-4-[4-(3-benzyloxy-propoxy)-phenyl]-5-hydroxy-3-(naphthalen-2-ylmethoxy)-piperidine-1-carboxylate by means of zinc bromide in methylene chloride, (3RS,4SR,5RS)-4-[4-(3-benzyloxy-propoxy)-phenyl]-3-(naphthalen-2-ylmethoxy)-piperidin-5-ol as a colorless solid; MS: 499 (M+H)⁺;

2)—from tert-butyl (RS)-4-[4-(3-benzyloxy-propoxy)-phenyl]-3-(naphthalen-2-ylmethoxy)-3,6-dihydro-2H-pyridine-1-carboxylate by means of hydrogen chloride in methanol, (RS)-4-[4-(3-benzyloxy-propoxy)-phenyl]-3-(naphthalen-2-ylmethoxy)-1,2,3,6-tetrahydro-pyridine in the form of a colorless oil; MS:480 (M+H)⁺;

3)—from tert-butyl (3RS,4SR,5SR)-5-amino-4-[4-(3-benzyloxy-propoxy)-phenyl]-3-(naphthalen-2-ylmethoxy)-piperidine-1-carboxylate by means of zinc bromide in methylene chloride, (3RS,4SR,5SR)-4-[4-(3-benzyloxy-propoxy)-phenyl]-3-(naphthalen-2-ylmethoxy)-piperidin-5-ylamine in the form of a colorless solid; MS: 497 (M+H)⁺.

4)—from a mixture of tert-butyl (3RS,4RS,5SR)- and (3SR,4SR,5RS)-4-[4-(3-benzyloxy-propoxy)-phenyl]-3-(quinolin-7-ylmethoxy)-5-[2-[(RS)-tetrahydro-pyran-2-yloxy]-ethoxy]-piperidine-1-carboxylate, 2-[4-(3RS,4RS,5SR)-[4-(3-benzyloxy-propoxy)-phenyl]-3-(quinolin-7-ylmethoxy)-piperidin-5-yloxy]-ethanol in the form of a colorless oil; MS: 543 (M+H)⁺;

5)—from a mixture of tert-butyl (3RS,4SR,5SR)- and (3SR,4RS,5RS)-4-[4-(3-benzyloxy-propoxy)-phenyl]-3-(quinolin-7-ylmethoxy)-5-[3-[(RS)-tetrahydro-pyran-2-yloxy]-propoxy]-piperidine-1-carboxylate, 3-[4-(3RS,4SR,5SR)-[4-(3-benzyloxy-propoxy)-phenyl]-3-(quinolin-7-ylmethoxy)-piperidin-5-yloxy]-propan-1-ol in the form of a colorless oil; MS: 557 (M+H)⁺;

6)—from a mixture of tert-butyl (3RS,4SR,5SR)- and (3SR,4RS,5RS)-4-[4-(3-benzyloxy-propoxy)-phenyl]-3-(quinolin-7-ylmethoxy)-5-[4-[(RS)-tetrahydro-pyran-2-yloxy]-butoxy]-piperidine-1-carboxylate, 4-[4-(3RS,4SR,5SR)-[4-(3-benzyloxy-propoxy)-phenyl]-3-(quinolin-7-ylmethoxy)-piperidin-5-yloxy]-butan-1-ol in the form of a colorless oil; MS: 571 (M+H)⁺.

The BOC derivatives used as starting materials were prepared as follows:

(a) A solution of 850 mg (1.422 mmol) of tert-butyl-(3RS,4SR,5SR)-4-[4-(3-benzyloxy-propoxy)-phenyl]-5-hydroxy-3-(naphthalen-2-ylmethoxy)-piperidine-1-carboxylate [Example 109 (a)] and 1.8849 (7.11 mmol) of triphenylphosphine in 170 ml of dry tetrahydrofuran was treated while stirring with 274 μl (7.11 mmol) of dry formic acid and a solution of 1.238 g (7.11 mmol) of diethyl azodicarboxylate in 42.5 ml of tetra-hydrofuran. The reaction mixture was subsequently stirred at room temperature for 90 hours. Thereupon, it was evaporated under reduced pressure at 40° C. and the residue was treated with a solution of 42.5 ml of methanol and 464 mg (7.11 mmol) of potassium hydroxide and stirred at room temperature for 3 hours. Subsequently, the solution was treated with 500 ml of deionized water and the mixture was extracted four times with 200 ml of methylene chloride each time. The combined organic phases were dried over sodium sulfate, evaporated under reduced pressure at 40° C. and the residue was dried in a high vacuum. The white crystalline residue (4.2 g) was chromatographed on silica gel using a 4:1 mixture of methylene chloride and ethyl acetate as the eluent. There were obtained 560 mg (66% of theory) of tert-butyl (3RS,4SR,5RS)-4-[4-(3-benzyloxy-propoxy)-phenyl]-5-hydroxy-3-(naphthalen-2-ylmethoxy)-piperidine-1-carboxylate in the form of a colorless solid; MS: 598 (M+H)⁺.

(b) A solution of 560 mg (0.937 mmol) of tert-butyl (3RS,4SR,5RS)-4-[4-(3-benzyloxy-propoxy)-phenyl]-5-hydroxy-3-(naphthalen-2-ylmethoxy)-piperidine-1-carboxylate and 273 mg (1.031 mmol) of triphenylphosphine in 20 ml of dry tetrahydrofuran was treated while stirring with 160.2 .mu.1 (1.031 mmol) of diethyl azodicarboxylate and ten minutes later with a solution of 319.8 .mu.1 (1.405 mmol) of diphenylphosphoryl azide in 2 ml of tetrahydrofuran. This mixture was stirred at room temperature for 72 hours, then evaporated under reduced pressure at 40° C. and the residue was dried in a high vacuum. The yellow oily residue was chromatographed on silica gel using a 4:1 mixture of n-hexane and ethyl aceatate and the eluent. There were obtained 210 mg (36% of theory) of tert-butyl (3RS,4SR,5SR)-5-azido-4-[4-(3-benzyloxy-propoxy)-phenyl]-3-(naphthalen-2-ylmethoxy)-piperidine-1-carboxylate in the form of a colorless oil [MS: 623 (M+H⁺)]. As a further product there were obtained 180 mg (33.1% of theory) of tert-butyl (RS)-4-[4-(3-benzyloxy-propoxy)-phenyl]-3-(naphthalen-2-ylmethoxy)-3,6-dihydro-2H-pyridine-1-carboxylate, likewise as a colorless oil; MS: 580 (M+H)⁺.

(c) A solution of 50 mg (0.0803 mmol) of tert-butyl (RS)-4-[4-(3-benzyloxy-propoxy)-phenyl]-3-(naphthalen-2-ylmethoxy)-3,6-dihydro-2H-pyridine-1-carboxylate in 0.36 ml of dry tetra-hydrofuran was treated while stirring with 21 mg (0.0793 mmol) of triphenylphosphine dissolved in 0.36 ml of dry tetrahydrofuran. Thereupon, the mixture was stirred at room temperature for four hours (about 50% conversion) and subsequently again treated with 10.6 mg (0.040 mmol) of triphenylphosphine and finally stirred at room temperature for a further 24 h. Then, the mixture was treated with 2 .mu.1 (0.111 mmol) of deionized water and stirred at room temperature for one hour. Subsequently, the mixture was evaporated under reduced pressure at 40° C. and the residue was taken up in ether and extracted against water. The organic phase was dried over sodium sulfate and the filtrate was evaporated. The colorless oily residue (110 mg) was chromatographed on silica gel using a 4:1 mixture of n-hexane and ethyl acetate as the eluent. There were obtained 30 mg (63% of theory) of tert-butyl (3RS,4SR,5SR)-5-amino-4-[4-(3-benzyloxy-propoxy)-phenyl]-3-(naphthalen-2-ylmethoxy)-piperidine-1-carboxylate in the form of a colorless solid; MS: 597 (M+H)⁺.

The following BOC derivatives were obtained from tert-butyl (3RS,4SR,5SR)-4-[4-(3-benzyloxy-propoxy)-phenyl]-5-hydroxy-3-(quinolin-7-ylmethoxy)-piperidine-1-carboxylate as follows in an analogous manner to that described in Example 1 (g), but at a reaction temperature of 50° C.;

(d)—By alkylation with rac-2-(2-bromoethoxy)-tetrahydropyran [J. Amer. Chem. Soc. 70, 4187 (1948)], a mixture of tert-butyl (3RS,4RS,5SR)- and (3SR,4SR,5RS)-4-[4-(3-benzyloxy-propoxy)-phenyl]-3-(quinolin-7-ylmethoxy)-5-[2-[(RS)-tetrahydro-pyran-2-yloxy]-ethoxy]-piperidine-1-carboxylate as a colorless oil; MS: 727 (M+H)⁺;

(e)—by alkylation with rac-2-(3-bromo-propoxy)-tetrahydropyran [J. Chem. Soc. 1955, 1770], a mixture of tert-butyl (3RS,4SR,5SR)- and (3SR,4RS,5RS)-4-[4-(3-benzyloxy-propoxy)-phenyl]-3-(quinolin-7-ylmethoxy)-5-[3-[(RS)-tetrahydro-pyran-2-yloxy]-propoxy]-piperidine-1-carboxylate as a colorless oil; MS: 741 (M+H)⁺;

(f)—by alkylation with rac-2-(4-bromo-butoxy)-tetrahydropyran [S. W. Baldwin et al., J. Org. Chem. 1985, 50, 4432-4439], a mixture of tert-butyl (3RS,4SR,5SR)- and (3SR,4RS,5RS)-4-[4-(3-benzyloxy-propoxy)-phenyl]-3-(quinolin-7-ylmethoxy)-5-[4-[(RS)-tetrahydro-pyran-2-yloxy]-butoxy]-piperidine-1-carboxylate as a colorless oil; MS: 755 (M+H)⁺.

### Example 112

The following compounds were obtained by cleavage of the BOC group by means of zinc bromide in methyl ene chloride analogously to Example 10 (b):

1)—From tert-butyl (3R,4s,5S)-3,5-bis-(4-methoxy-benzyloxy)-4-{4-[3-(2-methoxy-benzyloxy)-propoxy]-phenyl}-piperidine-1-carboxylate, (3R,4s,5S)-4-[4-[3-(2-methoxy-benzyloxy)-propoxy)-phenyl]-3,5-bis-(naphthalen-2-ylmethoxy)-piperidine as a colorless oil; MS: 628 (M+H)⁺;

2)—from tert-butyl (3RS,4SR,5SR)-5-hydroxy-3-(4-methoxy-benzyloxy)-4-{4-[3-(2-methoxy-benzyloxy)-propoxy]-phenyl}-piperidine-1-carboxylate, (3RS,4SR,5SR)-3-(4-methoxy-benzyloxy)-4-{4-[3-(2-methoxy-benzyloxy)-propoxy]-phenyl}-piperidin-5-ol as a colorless oil; MS: 508 (M+H)⁺;

3)—from tert-butyl (3R,4s,5S)-4-[4-[3-(2-methoxy-benzyloxy)-propoxy]-phenyl]-3,5-bis-(pyridin-2-ylmethoxy)-piperidine-1-carboxylate, (3R,4s,5S)-2-[5-(pyridin-2-ylmethoxy-4-[4-[3-(2-methoxy-benzyloxy)-propoxy]-phenyl]-piperidin-3-yloxymethyl}-pyridine as a colorless oil; MS: 570 (M+H)⁺;

4)—from tert-butyl (3RS,4SR,5SR)-5-hydroxy-4-{4-[3-(2-methoxy-benzyloxy)-propoxy]-phenyl}-3-(pyridin-2-ylmethoxy)-piperidine-1-carboxylate, (3RS,4SR,5SR)-4-[4-[3-(2-methoxy-benzyloxy)-propoxy]-phenyl]-3-(pyridin-2-ylmethoxy)-piperidin-5-ol as a colorless oil; MS: 479 (M+H)⁺;

5)—from tert-butyl (3R,4s,5S)-4-[4-[3-(2-methoxy-benzyloxy)-propoxy]-phenyl]-3,5-bis-(pyridin-3-ylmethoxy)-piperidine-1-carboxylate, (3R,4s,5S)-3-[5-(pyridin-3-ylmethoxy-4-[4-[3-(2-methoxy-benzyloxy)-propoxy]-phenyl]-piperidin-3-yloxymethyl]-pyridine as a colorless oil; MS: 570 (M+H)⁺;

6)—from tert-butyl (3RS,4SR,5SR)-5-hydroxy-4-{4-[3-(2-methoxy-benzyloxy)-propoxy]-phenyl}-3-(pyridin-3-ylmethoxy)-piperidine-1-carboxylate, (3RS,4SR,5SR)-4-[4-[3-(2-methoxy-benzyloxy)-propoxy]-phenyl]-3-(pyridin-3-ylmethoxy)-piperidin-5-ol as a colorless oil; MS: 479 (M+H)⁺;

7)—from tert-butyl (3R,4s,5S)-4-[4-[3-(2-methoxy-benzyloxy)-propoxy]-phenyl]-3,5-bis-(pyridin-4-ylmethoxy)-piperidine-1-carboxylate, (3R,4s,5S)-4-[5-(pyridin-4-ylmethoxy-4-[4-[3-(2-methoxy-benzyloxy)-propoxy]-phenyl]-piperidin-3-yloxymethyl]-pyridine as a colorless oil; MS: 570 (M+H)⁺;

8)—from tert-butyl (3RS,4SR,5SR)-5-hydroxy-4-{4-[3-(2-methoxy-benzyloxy)-propoxy]-phenyl}-3-(pyridin-4-ylmethoxy)-piperidine-1-carboxylate, (3RS,4SR,5SR)-4-[4-[3-(2-methoxy-benzyloxy)-propoxy]-phenyl]-3-(pyridin-4-ylmethoxy)-piperidin-5-ol as a colorless oil; MS: 479 (M+H)⁺;

9)—from (3RS,4SR,5SR)-5-hydroxy-4-[4-[3-(2-methoxy-benzyloxy)-propoxy]-phenyl]-3-[7-[2-(4-methyl-piperazin-1-yl)-ethoxy)-naphthalen-2-ylmethoxy]-piperidin-1-carboxylic acid tert-butylester. 1-[2-[7-[(3RS,4SR,5SR)-5-hydroxy-4-[4-[3-(2-methoxy-benzyloxy)-propoxy]-piperidin-3-yloxymethyl]-naphthalen-2-yloxy]-ethyl]-4-methyl-piperazine as an amorphous colorless solid, MS: 670 (M+H)⁺;

10)—from (3R,4s,5S)-3,5-bis-(1,4-dimethoxy-naphthalen-2-ylmethoxy)-4-[4-[3-(2-methoxy-benzyloxy)-propoxy]-phenyl]-piperidin-1-carboxylic acid tert-butylester by means of zinc bromide in methylene chloride, (3R,4s,5S)-3,5-bis-(1,4-dimethoxy-naphthalen-2-ylmethoxy)-4-[4-[3-(2-methoxy-benzyloxy)-propoxy)-phenyl]-piperidine as a colorless wax; MS: 788 (M+H)⁺;

11)—from (3RS,4SR,5SR)-3-(1,4-dimethoxy-naphthalen-2-ylmethoxy)-5-hydroxy-4-[4-[3-(2-methoxy-benzyloxy)-propoxy]-phenyl]-piperidin-1-carboxylic acid tert-butylester by means of zinc bromide in methylene chloride, (3RS,4SR,5SR)-3-(1,4-dimethoxy-naphthalen-2-ylmethoxy)-4-[4-[3-(2-methoxy-benzyloxy)-propoxy]-phenyl]-piperidin-5-ol as a colorless oil; MS: 588 (M+H)⁺.

The BOC derivatives used as starting materials were obtained as follows:

(α) In an analogous manner to that described in Example 44 (e), by alkylating tert-butyl (3R,4s,5S)-3,5-dihydroxy-4-(4-hydroxy-phenyl)-piperidine-1-carboxylate [Example 109 (h)] with 2-methoxybenzyl 3-chloropropyl ether [Example 120 (g)] there was obtained tert-butyl (3R,4s,5S)-3,5-dihydroxy-4-{4-[3-(2-methoxy-benzyloxy)-propoxy]-phenyl}-piperidine-1-carboxylate in the form of an amorphous, colorless solid; MS: 505 (M+NH₄)⁺.

(β) In an analogous manner to that described in Example 1 (g), by alkylating tert-butyl (3R,4s,5S)-3,5-dihydroxy-4-{4-[3-(2-methoxy-benzyloxy)-propoxy]-phenyl}-piperidine-1-carboxylate using one equivalent of a benzylic halide there were obtained about equal proportions of unreacted starting material and of the corresponding mono- und dialkylated BOC derivatives. These mixtures were subsequently separated by chromatography:

(a)—By alkylation with 4-methoxy-benzyl chloride, tert-butyl (3R,4s,5S)-3,5-bis-(4-methoxy-benzyloxy)-4-{4-[3-(2-methoxy-benzyloxy)-propoxy]-phenyl}-piperidine-1-carboxylate, MS: 746 (M+NH₄)⁺, and tert-butyl (3RS,4SR,5SR)-5-hydroxy-3-(4-methoxy-benzyloxy)-4-{4-[3-(2-methoxy-benzyloxy)-propoxy]-phenyl}-piperidine-1-carboxylate, MS: 626 (M+NH₄)⁺, each as an amorphous, colorless solid;

(b)—by alkylation with 2-chloromethyl-pyridine hydrochloride and corresponding excess of base, tert-butyl (3R,4s,5S)-4-[4-(3-(2-methoxy-benzyloxy)-propoxy]-phenyl]-3,5-bis-(pyridin-2-ylmethoxy)-piperidine-1-carboxylate, MS: 670 (M+H)⁺, and tert-butyl (3RS,4SR,5SR)-5-hydroxy-4-{4-[3-(2-methoxy-benzyloxy)-propoxy)-phenyl}-3-(pyridin-2-ylmethoxy)-piperidine-1-carboxylate, MS: 579 (M+H)⁺, each as an amorphous, colorless solid;

(c)—by alkylation with 3-chloromethyl-pyridine hydrochloride and corresponding excess of base, tert-butyl (3R,4s,5S)-4-[4-[3-(2-methoxy-benzyloxy)-propoxy]-phenyl]-3,5-bis-(pyridin-3-ylmethoxy)-piperidine-1-carboxylate, MS: 670 (M+H)⁺, and tert-butyl (3RS,4SR,5SR)-5-hydroxy-4-{4-[3-(2-methoxy-benzyloxy)-propoxy]-phenyl}-3-(pyridin-3-ylmethoxy)-piperidine-1-carboxylate, MS: 579 (M+H)⁺, each as an amorphous, colorless solid;

(d)—by alkylation with 4-chloromethyl-pyridine hydrochloride and corresponding excess of base, tert-butyl (3R,4s,5S)-4-[4-[3-(2-methoxy-benzyloxy)-propoxy)-phenyl]-3,5-bis-(pyridin-4-ylmethoxy)-piperidine-1-carboxylate, MS: 670 (M+H)⁺, and tert-butyl (3RS,4SR,5SR)-5-hydroxy-4-{4-[3-(2-methoxy-benzyloxy)-propoxy]-phenyl}-3-(pyridin-4-ylmethoxy)-piperidine-1-carboxylate, MS: 579 (M+H)⁺, each as an amorphous, colorless solid.

(e)—by alkylating (3R,4s,5S)-3,5-dihydroxy-4-{4-[3-(2-methoxy-benzyloxy)-propoxy]-phenyl}-piperidin-1-carboxylic acid tert-butylester [Example 12 (α)] with 2-chloromethyl-7-(2-trimethylsilanyl-ethoxymethoxy)-naphthalen [Example 6 (u)] there was obtained (3RS,4SR,5SR)-5-hydroxy-4-[4-[3-(2-methoxy-benzyloxy)-propoxy]-phenyl]-3-[7-(2-trimethylsilanyl-ethoxymethoxy)-naphthalen-2-ylmethoxy]-piperidin-1-carboxylic acid tert-butylester as a pale yellow oil; MS: 774 (M+H)⁺. This was then reacted in analogy to Example 95 (b) by spliting off the SEM-protecting group to afford (3RS,4SR,5SR)-5-hydroxy-4-[4-[3-(2-methoxy-benzyloxy)-propoxy]-phenyl]-3-(7-hydroxy-naphthalen-2-ylmethoxy)-piperidin-1-carboxylic acid tert-butylester [yellow oil; MS: 644 (M+H)⁺] which on alkylation with 1-(2-chloro-ethyl)-4-methyl-piperazine hydrochloride (1:2) [Chim. Ther. 4, 283 (1969)] in analogy to Example 90 (n) gave (3RS,4SR,5SR)-5-hydroxy-4-[4-[3-(2-methoxy-benzyloxy)-propoxy]-phenyl]-3-[7-[2-(4-methyl-piperazin-1-yl)-ethoxy]-naphthalen-2-ylmethoxy]-piperidin-1-carboxylic acid tert-butylester as a light brown oil; MS: 770 (M+H)⁺;

(f)—by alkylating (3R,4s,5S)-3,5-dihydroxy-4-{4-[3-(2-methoxy-benzyloxy)-propoxy]-phenyl}-piperidin-1-carboxylic acid tert-butylester (Example 112 (a)] with 2-chloromethyl-1,4-dimethoxy-naphthalene [J. Org. Chem. (1983), 48(19),3265-3268] there was obtained (3R,4s,5S)-3,5-bis-(1,4-dimethoxy-naphthalen-2-ylmethoxy)-4-[4-[3-(2-methoxy-benzyloxy)-propoxy]-phenyl]-piperidin-1-carboxylic acid tert-butylester, MS: 906 (M+H)⁺, as a colorless foam and (3RS,4SR,5SR)-3-(1,4-dimethoxy-naphthalen-2-ylmethoxy)-5-hydroxy-4-[4-[3-(2-methoxy-benzyloxy)-propoxy]-phenyl]-piperidin-1-carboxylic acid tert-butylester, MS:705 (M+NH₄)⁺, as a colorless oil.

### Example 113

The following compounds were obtained by cleavage of the BOC group by means of zinc bromide in methylene chloride analogously to Example 10 (b):

1)—From tert-butyl (3RS,4SR,5SR)-4-[4-(3-benzyloxy-propoxy)-phenyl]-5-hydroxy-3-(4-hydroxy-benzyloxy)-piperidine-1-carboxylate, (3RS,4SR,5SR)-4-[4-(3-benzyloxy-propoxy)-phenyl]-3-(4-hydroxy-benzyloxy)-piperidin-5-ol as a colorless oil; MS: 464 (M+H)⁺;

2)—from tert-butyl (3R,4s,5S)-4-[4-(3-benzyloxy-propoxy)-phenyl]-3,5-bis-(4-hydroxy-benzyloxy)-piperidine-1-carboxylate, 4-[(3R,4s,5S)-5-(4-hydroxybenzyloxy)-4-[4-(3-benzyloxy-propoxy)-phenyl]-piperidin-3-yloxymethyl}-phenol as a colorless oil; MS: 570 (M+H)⁺.

The BOC derivatives used as starting materials were obtained as follows:

(a) (α) In an analogous manner to that described in Example 1 (g), by alkylating tert-butyl (3R,4s,5S)-4-[4-(3-benzyloxy-propoxy)-phenyl]-3,5-dihydroxy-piperidine-1-carboxylate [Example 109 (q)] with 1-chloromethyl-4-(2-trimethylsilanyl-ethoxymethoxy)-benzene there were obtained tert-butyl (3RS,4SR,5SR)-4-[4-(3-benzyloxy-propoxy)-phenyl]-5-hydroxy-3-[4-(2-trimethylsilanyl-ethoxymethoxy)-benzyloxy]-piperidine-1-carboxylate, R_{f}: 0.33 (SiO₂, n-hexane:ethyl acetate=2:1), and tert-butyl (3R,4s,5S)-4-[4-(3-benzyloxy-propoxy)-phenyl]-3,5-bis-[4-(2-trimethylsilanyl-ethoxymethoxy)-benzyloxy]-piperidine-1-carboxylate, R_{f}: 0.64 (SiO₂, n-hexane:ethyl acetate=2:1), each as an amorphous, colorless solid

(a) (β) A solution of 2.16 g (3.113 mmol) of tert-butyl (3RS,4SR,5SR)-4-[4-(3-benzyloxy-propoxy)-phenyl]-5-hydroxy-3-[4-(2-trimethylsilanyl-ethoxymethoxy)-benzyloxy]-piperidine-1-carboxylate in 50 ml of methanol was treated with 2.02 ml (4.046 mmol) of an anhydrous 2M hydrogen chloride solution in methanol and stirred at room temperature for 2 hours. Subsequently, the mixture was treated with 100 ml of a 95:5 mixture of methylene chloride and methanol (extracted against 5 volume % of a saturated aqueous ammonia solution) and the solution was evaporated on a rotary evaporator at 30° C. The white solid (2.17 g) was thereupon chromatographed on silica gel using a 4:1 mixture of methylene chloride and ethyl acetate as the eluent. There were obtained 780 mg (45% of theory) of tert-butyl (3RS,4SR,5SR)-4-[4-(3-benzyloxy-propoxy)-phenyl]-5-hydroxy-3-(4-hydroxy-benzyloxy)-piperidine-1-carboxylate in the form of a colorless oil; MS: 581 (M+NH₄)⁺.

(b) A solution of 1.1 g (1.18 mmol) of tert-butyl(3R,4s,5S)-4-[4-(3-benzyloxy-propoxy)-phenyl]-3,5-bis-[4-(2-trimethylsilanyl-ethoxymethoxy)-benzyloxy]-piperidine-1-carboxylate in 20 ml of methanol was treated with 1.30 ml (2.60 mmol) of an anhydrous 2M hydrogen chloride solution in methanol and stirred at room temperature for 70 minutes. Subsequently, the mixture was treated with 50 ml of a 95:5 mixture of methylene chloride and methanol (extracted against 5 volume % of a saturated aqueous ammonia solution) and the solution was evaporated on a rotary evaporator at 30° C. The white solid (920 mg) was thereupon chromatographed on silica gel using a 4:1 mixture of methylene chloride and ethyl acetate as the eluent. There were obtained 300 mg (38% of theory) of tert-butyl (3R,4s,5S)-4-[4-(3-benzyloxy-propoxy)-phenyl]-3,5-bis-(4-hydroxy-benzyloxy)-piperidine-1-carboxylate in the form of a colorless oil; R_{f}: 0.26 (SiO₂, n-hexane:ethyl acetate=1:1).

The 1-chloromethyl-4-(2-trimethylsilanyl-ethoxymethoxy)-benzene used at the alkylating agent was prepared in analogy to Example 5 (a)-(c) from methyl 4-hydroxybenzoate by introducing the SEM group to give methyl 4-(2-trimethylsilanylethoxymethyl)-benzoate. Subsequent reduction with lithium aluminium hydride gave [4-(2-trimethylsilanylethoxy-methoxy)-phenyl]-methanol and chlorination of this gave 1-chlormethyl-4-(2-trimethylsilanyl-ethoxymethoxy)-benzene as a colorless oil; MS:272 (M)⁺.

### Example 114

The following compounds were obtained in analogy to the procedure described in Example 1 (e) by cleavage of the SEM group by means of tetrabutylammonium fluoride in tetrahydrofuran:

1)—From 2-trimethylsilylethyl (3RS,4RS)-4-(4-fluoro-phenyl)-3-[4-(2-morpholin-4-yl-ethoxy)-naphthalen-2-ylmethoxy]-piperidine-1-carboxylate, (3RS,4RS)-4-(4-fluoro-phenyl)-3-[4-(2-morpholin-4-yl-ethoxy)-naphthalen-2-ylmethoxy]-piperidine as a colorless solid; MS: 465 (M)⁺;

2)—from 2-trimethylsilylethyl (3RS,4RS)-4-(4-fluoro-phenyl)-3-[8-(3-methoxy-propoxy)-naphthalen-2-ylmethoxy)-piperidine-1-carboxylate, (3RS,4RS)-4-(4-fluoro-phenyl)-3-[8-(3-methoxy-propoxy)-naphthalen-2-ylmethoxy]-piperidine as a colorless solid; MS: 424 (M)⁺;

3)—from 2-trimethylsilylethyl (3RS,4RS)-4-(4-fluoro-phenyl)-3-[5-(3-methoxy-propoxy)-naphthalen-2-ylmethoxy]-piperidine-1-carboxylate, (3RS,4RS)-4-(4-fluoro-phenyl)-3-[5-(3-methoxy-propoxy)-naphthalen-2-ylmethoxy]-piperidine as a colorless solid; MS: 424 (M)⁺;

4)—from 2-trimethylsilylethyl (3RS,4RS)-4-(4-fluoro-phenyl)-3-[7-(3-methoxy-propoxy)-naphthalen-2-ylmethoxy]-piperidine-1-carboxylate, (3RS,4RS)-4-(4-fluoro-phenyl)-3-[7-(3-methoxy-propoxy)-naphthalen-2-ylmethoxy]-piperidine as a colorless solid; MS: 424 (M)⁺.

The SEM derivatives used as starting materials were prepared as follows:

(a)—From 2-trimethylsilylethyl (3RS,4RS)-4-(4-fluorophenyl)-3-(4-hydroxy-naphthalen-2-ylmethoxy)-piperidine-1-carboxylate [Example 5 (g)] and 4-(2-chloroethyl)-morpholine hydrochloride, 2-trimethylsilylethyl (3RS,4RS)-4-(4-fluoro-phenyl)-3-[4-(2-morpholin-4-yl-ethoxy)-naphthalen-2-ylmethoxy]-piperidine-1-carboxylate as a yellow oil; MS: 609 (M)⁺;

(b)—from 2-trimethylsilylethyl (3RS,4RS)-4-(4-fluorophenyl)-3-(8-hydroxy-naphthalen-2-ylmethoxy)-piperidine-1-carboxylate [Example 6 (dd)] and 1-chloro-3-methoxypropane, 2-trimethylsilylethyl (3RS,4RS)-4-(4-fluoro-phenyl)-3-[8-(3-methoxy-propoxy)-naphthalen-2-ylmethoxy]-piperidine-1-carboxylate as a light yellow oil; MS: 568 (M)⁺;

(c)—from 2-trimethylsilylethyl (3RS,4RS)-4-(4-fluorophenyl)-3-(5-hydroxy-naphthalen-2-ylmethoxy)-piperidine-1-carboxylate [Example 6 (1)] and 1-chloro-3-methoxypropane, 2-trimethylsilylethyl (3RS,4RS)-4-(4-fluoro-phenyl)-3-[5-(3-methoxy-propoxy)-naphthalen-2-ylmethoxy]-piperidine-1-carboxylate as a colorless resin, which was used in the next step without further purification and characterization;

(d)—from 2-trimethylsilylethyl (3RS,4RS)-4-(4-fluorophenyl)-3-(7-hydroxy-naphthalen-2-ylmethoxy)-piperidine-1-carboxylate [Example 6 (x)] and 1-chloro-3-methoxypropane, 2-trimethylsilylethyl (3RS,4RS)-4-(4-fluoro-phenyl)-3-[7-(3-methoxy-propoxy)-naphthalen-2-ylmethoxy]-piperidine-1-carboxylate as a colorless resin, which was used in the next step without further purification and characterization.

### Example 115

(3RS,4RS)-4-(4-Fluorophenyl)-3-[4-β-hydroxy-benzyloxy]-naphthalen-2-ylmethoxy]-piperidine was obtained as follows in an analogous manner to that described in Example 5:

(a) In an analogous manner to that described in Example 5 (a)-(d), firstly from ethyl 3-hydroxybenzoate by introduction of the SEM group there was obtained ethyl 3-(2-trimethylsilyl-ethoxymethoxy)-benzoate as a colorless oil; MS 238 [M-(C₂H₄+CH₂O)]⁺. Subsequent reduction gave [3-(2-trimethylsilylethoxy-methoxy)-phenyl]-methanol, MS: 196 [M-(C₂H₄+CH₂O)]⁺, as a colorless oil, chlorination of which yielded 1-chloromethyl-3-(2-trimethylsilyl-ethoxymethoxy)-benzene as a colorless oil; MS: 214, 216 [M-(C₂H₄+CH₂O)]⁺. Subsequent alkylation of 2-trimethylsilylethyl (3RS,4RS)-4-(4-fluorophenyl)-3-(4-hydroxy-naphthalen-2-ylmethoxy)-piperidine-1-carboxylate [Example 5 (g)] with 1-chloromethyl-3-(2-trimethylsilyl-ethoxymethoxy)-benzene yielded 2-trimethylsilylethyl (3RS,4RS)-4-(4-fluorophenyl)-3-[4-[3-(2-trimethylsilyl-ethoxymethoxy)-benzyloxy]-naphthalen-2-ylmethoxy]-piperidine-1-carboxylate as a light yellow oil; MS: 749 (M+NH₄)⁺.

(b) From 2-trimethylsilylethyl (3RS,4RS)-4-(4-fluorophenyl)-3-[4-[3-(2-trimethylsilyl-ethoxymethoxy)-benzyloxy]-naphthalen-2-ylmethoxy]-piperidine-1-carboxylate there was obtained in analogy to the procedure described in Example 1 (e) by cleavage of the 2-trimethylsilylethyl carbamate with tetrabutylammonium fluoride in tetrahydrofuran (3RS,4RS)-4-(4-fluorophenyl)-3-[4-[3-(2-trimethylsilyl-ethoxymethoxy)-benzyloxy 1-naphthalen-2-ylmethoxy]-piperidine, MS: 588 (M+H)⁺, as a light yellow oil, from which by cleavage of the SEM group by means of a 2N solution of hydrogen chloride in methanol analogously to the procedure described in Example 5 (g) there was obtained (3RS,4RS)-4-(4-fluorophenyl)-3-[4-β-hydroxy-benzyloxy]-naphthalen-2-ylmethoxy]-piperidine as a colorless solid; MS: 458 (M+H)⁺.

### Example 116

The following compound was obtained in an analogous manner to that described in Example 10 (b) by cleavage of the BOC group by means of zinc bromide in methylene chloride:

From tert-butyl (3RS,4RS)-3-(naphthalen-2-ylmethoxy)-4-[4-[2-(5-phenyl-[1,3,4]oxadiazol-2-yl)-ethyl]-phenyl]-piperidine-1-carboxylate by means of zinc bromide in methylene chloride, (3RS,4RS)-3-(naphthalen-2-ylmethoxy)-4-[4-[2-(5-phenyl-[1,3,4)oxadiazol-2-yl)-ethyl]-phenyl)-piperidine as a colorless solid; MS: 490 (M+H).

The BOC derivative used as the starting material was prepared as follows:

(a) From tert-butyl (3RS,4RS)-4-[4-(2-carboxy-ethyl)-phenyl]-3-(naphthalen-2-ylmethoxy)piperidine-1-carboxylate [Example 86 (II)] by condensation with benzoic acid hydrazide in the presence of EDC in an analogous manner to that described in Example 38, tert-butyl (3RS,4RS)-4-[4-[3-(N′-benzoyl-hydrazino)-3-oxo-propyl]-phenyl]-3-(naphthalen-2-ylmethoxy)-piperidine-1-carboxylate as a colorless resin; MS: 608 (M+H)⁺.

(b) A solution of 106 mg (0.174 mmol) of tert-butyl (3RS,4RS)-4-[4-[3-(N′-benzoyl-hydrazino)-3-oxo-propyl]-phenyl]-3-(naphthalen-2-ylmethoxy)-piperidine-1-carboxylate in 1.5 ml of hexamethyldisilazane was treated with 39 μl (0.039 mmol) of a 1M solution of tetrabutylammonium fluoride in tetrahydrofuran and heated to reflux for 20 hours. For the working-up, the reaction mixture was treated with 50 ml of a 1:1 mixture of methylene chloride and water, the organic phase was subsequently separated and the aqueous phase was back-extracted twice with 25 ml of methylene chloride each time. The combined organic phases were dried over sodium sulfate and evaporated under reduced pressure. The crude product was purified by flash chromatography on silica gel with a 7:3 mixture of hexane and ethyl acetate as the eluent. There were obtained 70 mg (68% of theory) of tert-butyl (3RS,4RS)-3-(naphthalen-2-ylmethoxy)-4-[4-[2-(5-phenyl-[1,3,4]oxadiazol-2-yl)-ethyl]-phenyl]-piperidine-1-carboxylate as a colorless solid; MS: 590 (M+H)⁺.

### Example 117

The following compound was obtained in an analogous manner to that described in Example 22 (l) by cleavage of the BOC group by means of hydrogen chloride in methanol:

From tert-butyl (3RS,4RS)-4-[4-(3-benzyloxy-propyl)-phenyl]-3-(naphthalen-2-ylmethoxy)-piperidine-1-carboxylate, (3RS,4RS)-4-[4-(3-benzyloxy-propyl)-phenyl]-3-(naphthalen-2-ylmethoxy)-piperidine as a colorless, amorphous solid; MS: 467 (M+H)⁺.

The BOC derivative used as the starting material was prepared as follows:

In an analogous manner to that described in Example 1 (g), by alkylating tert-butyl (3RS,4RS)-4-[4-(3-hydroxy-propyl)-phenyl]-3-(naphthalen-2-ylmethoxy)-piperidine-1-carboxylate [Example 24 (t)] with benzyl bromide therer was obtained tert-butyl (3RS,4RS)-4-[4-(3-benzyloxy-propyl)-phenyl]-3-(naphthalen-2-ylmethoxy)-piperidine-1-carboxylate as a colorless, amorphous solid, which was used in the next step without characterization.

### Example 118

The following compounds were prepared by cleavage of the BOC group by means of hydrogen chloride in methanol analogously to Example 22 (l):

1)—From tert-butyl (3RS,4RS)-3-(naphthalen-2-ylmethoxy)-4-[4-(2-phenoxy-ethoxymethyl)-phenyl]-piperidine-1-carboxylate, (3RS,4RS)-3-(naphthalen-2-ylmethoxy)-4-[4-(2-phenoxy-ethoxymethyl)-phenyl]-piperidine as a light yellow oil; MS: 468 (M+H)⁺;

2)—from tert-butyl (3RS,4RS)-3-(naphthalen-2-ylmethoxy)-4-[4-(3-phenoxy-propoxymethyl)-phenyl]-piperidine-1-carboxylate, (3RS,4RS)-3-(naphthalen-2-ylmethoxy)-4-[4-(3-phenoxy-propoxymethyl)-phenyl]-piperidine as a colorless foam; MS: 482 (M+H)⁺;

3)—from tert-butyl (3RS,4RS)-4-[4-(3-benzyloxy-propoxy-methyl)-phenyl]-3-(naphthalen-2-ylmethoxy)-piperidine-1-carboxylate, (3RS,4RS)-4-[4-(3-benzyloxy-propoxymethyl)-phenyl]-3-(naphthalen-2-ylmethoxy)-piperidine as a light yellow oil; MS: 496 (M+H)⁺;

4)—from tert-butyl (3RS,4RS)-4-[4-[3-(4-fluoro-phenoxy)-propoxymethyl)-phenyl]-3-(naphthalen-2-ylmethoxy)-piperidine-1-carboxylate, (3RS,4RS)-4-[4-[3-(4-fluoro-phenoxy)-propoxy-methyl]-phenyl]-3-(naphthalen-2-ylmethoxy)-piperidine as a light yellow oil; MS: 500 (M+H)⁺.

The BOC derivatives used as starting materials were prepared as follows by alkylating tert-butyl (3RS,4RS)-4-(4-hydroxymethyl-phenyl)-3-(naphthalen-2-ylmethoxy)-piperidine-1-carboxylate [Example 22 (j)] in an analogous manner to that described in Example 1 (g):

(a)—By alkylation with b-bromophenetol, tert-butyl (3RS,4RS)-3-(naphthalen-2-ylmethoxy)-4-[4-(2-phenoxy-ethoxymethyl)-phenyl]-piperidine-1-carboxylate, as a colorless resin, which was used in the next step without further characterization;

(b)—by alkylation with 3-phenoxypropyl bromide, tert-butyl (3RS,4RS)-3-(naphthalen-2-ylmethoxy)-4-[4-(3-phenoxy-propoxymethyl)-phenyl]-piperidine-1-carboxylate as a colorless oil; MS: 582 (M+H)⁺;

(c)—by alkylation with benzyl 3-bromopropyl ether, tert-butyl (3RS,4RS)-4-[4-(3-benzyloxy-propoxymethyl)-phenyl]-3-(naphthalen-2-ylmethoxy)-piperidin-1-carboxylate as a colorless resin; MS: 596 (M+H)⁺;

(d)—by alkylation with 1-(3-chloropropoxy)-4-fluorobenzene, tert-butyl (3RS,4RS)-4-[4-[3-(4-fluoro-phenoxy)-propoxymethyl]-phenyl]-3-(naphthalen-2-ylmethoxy)-piperidine-1-carboxylate as a colorless resin; MS: 600 (M+H)⁺.

### Example 119

The following compounds were obtained by cleavage of the BOC group by means of hydrogen chloride in methanol analogously to Example 22 (l) or by means of zinc bromide in methylene chloride analogously to Example 10 (b):

1)—From tert-butyl (3RS,4RS)-4-[4-(3-benzyloxy-propoxy)-phenyl]-3-(8-hydroxy-naphthalen-2-ylmethoxy)-piperidine-1-carboxylate by means of hydrogen chloride in methanol, (3RS,4RS)-4-[4-(3-benzyloxy-propoxy)-phenyl]-3-(8-hydroxy-naphthalen-2-ylmethoxy)-piperidine as a colorless solid; MS: 498 (M+H)⁺;

2)—from tert-butyl (3RS,4RS)-4-[4-(3-benzyloxy-propoxy)-phenyl]-3-[8-(3-methoxy-ethoxy)-naphthalen-2-ylmethoxy]-piperidine-1-carboxylate by means of hydrogen chloride in methanol, (3RS,4RS)-4-[4-(3-benzyloxy-propoxy)-phenyl]-3-[8-(3-methoxy-ethoxy)-naphthalen-2-ylmethoxy]-piperidine as a light yellow oil; MS: 556 (M+H)⁺;

3)—from tert-butyl (3RS,4RS)-4-[4-(3-benzyloxy-propoxy)-phenyl]-3-[8-(3-methoxy-propoxy)-naphthalen-2-ylmethoxy]-piperidine-1-carboxylate by means of hydrogen chloride in methanol, (3RS,4RS)-4-[4-(3-benzyloxy-propoxy)-phenyl]-3-[8-(3-methoxy-propoxy)-naphthalen-2-ylmethoxy]-piperidine as a light yellow oil; MS: 570 (M+H)⁺;

4)—from tert-butyl (3RS,4RS)-4-[4-(3-benzyloxy-propoxy)-phenyl]-3-(8-methoxycarbonylmethoxy-naphthalen-2-ylmethoxy)-piperidine-1-carboxylate by means of hydrogen chloride in methanol, (3RS,4RS)-4-[4-(3-benzyloxy-propoxy)-phenyl]-3-(8-methoxycarbonylmethoxy-naphthalen-2-ylmethoxy)-piperidine as a colorless solid; MS: 570 (M+H)⁺;

5)—from tert-butyl (3RS,4RS)-4-[4-(3-benzyloxy-propoxy)-phenyl]-3-(8-carbamoylmethoxy-naphthalen-2-ylmethoxy)-piperidine-1-carboxylate by means of zinc bromide in methylene chloride, (3RS,4RS)-4-[4-(3-benzyloxy-propoxy)-phenyl]-3-(8-carbamoylmethoxy-naphthalen-2-ylmethoxy)-piperidine as a colorless foam; MS: 555 (M+H)⁺;

The BOC derivatives used as starting materials were prepared as follows by alkylating tert-butyl (3RS,4RS)-4-[4-(3-benzyloxy-propoxy)-phenyl]-3-(8-hydroxy-naphthalen-2-ylmethoxy)-piperidine-1-carboxylate in an analogous manner to that described in Examples 1 and 5:

(a)—By alkylation with 2-methoxyethyl bromide, tert-butyl (3RS,4RS)-4-[4-(3-benzyloxy-propoxy)-phenyl]-3-[8-(3-methoxy-ethoxy)-naphthalen-2-ylmethoxy]-piperidine-1-carboxylate as a colorless resin, which was used in the next step without characterization;

(b)—by alkylation with 1-chloro-3-methoxypropane, tert-butyl (3RS,4RS)-4-[4-(3-benzyloxy-propoxy)-phenyl]-3-[8-(3-methoxy-propoxy)-naphthalen-2-ylmethoxy]-piperidine-1-carboxylate as a light yellow resin, which was used in the next step without characterization;

(c)—by alkylation with methyl bromoacetate, tert-butyl (3RS,4RS)-4-[4-(3-benzyloxy-propoxy)-phenyl]-3-(8-methoxycarbonylmethoxy-naphthalen-2-ylmethoxy)-piperidine-1-carboxylate as a colorless solid; MS: 670 (M+H)⁺;

(d)—by alkylation with iodoacetamide, tert-butyl (3RS,4RS)-4-[4-(3-benzyloxy-propoxy)-phenyl]-3-(8-carbamoylmethoxy-naphthalen-2-ylmethoxy)-piperidine-1-carboxylate as a light yellow resin, which was used in the next step without characterization;

The tert-butyl (3RS,4RS)-4-[4-(3-benzyloxy-propoxy)-phenyl]-3-(8-hydroxy-naphthalen-2-ylmethoxy)-piperidine-1-carboxylate used as the starting material was prepared as follows as described in Examples 1, 5 and 6:

(a) By alkylating tert-butyl (3RS,4RS)-4-[4-(3-benzyloxy-propoxy)-phenyl]-3-hydroxy-piperidine-1-carboxylate [Example 86 (n)] with 2-chloromethyl-8-(2-trimethylsilylethoxy-methoxy)-naphthalene [Example 6 (aa)] there was obtained tert-butyl (3RS,4RS)-4-[4-(3-benzyloxy-propoxy)-phenyl]-3-[8-(2-trimethylsilanyl-ethoxymethoxy)-naphthalen-2-ylmethoxy-piperidine-1-carboxylate as a light yellow oil; MS: 728 (M+H)⁺.

(b) A solution of 552 mg of tert-butyl (3RS,4RS)-4-[4-(3-benzyloxy-propoxy)-phenyl]-3-[8-(2-trimethylsilanyl-ethoxy-methoxy)-naphthalen-2-ylmethoxypiperidine-1-carboxylate in 4 ml of methanol was treated with 4 ml of a 2N solution of hydrogen chloride in methanol and stirred at room temperature for 45 minutes. For the working-up, the mixture was partitioned between 50 ml of ethyl acetate and 50 ml of aqueous 5% sodium hydrogen carbonate solution and then the organic phase was separated. The aqueous phase was extracted three times with 25 ml of ethyl acetate each time. The combined ethyl acetate phases were dried over magnesium sulfate and finally the solvent was distilled off under reduced pressure. The crude product (538 mg) was purified by chromatography on silica gel with a 1:2 mixture of ethyl acetate and hexane as the eluent. There were obtained 348 mg (77% of theory) of tert-butyl (3RS,4RS)-4-[4-(3-benzyloxy-propoxy)-phenyl]-3-(8-hydroxy-naphthalen-2-ylmethoxy)-piperidine-1-carboxylate as a colorless foam; MS: 598 (M+H)⁺.

### Example 120

The following compounds were prepared by cleavage of the BOC group by means of hydrogen chloride in methanol analogously to Example 22 (l):

1)—From tert-butyl (3RS,4RS)-4-[4-(3-benzyloxy-propoxy)-phenyl]-3-(quinolin-7-ylmethoxy)-piperidine-1-carboxylate, (3RS,4RS)-4-[4-(3-benzyloxy-propoxy)-phenyl]-3-(quinolin-7-ylmethoxy)-piperidine as a colorless oil; MS: 483 (M+H)⁺;

2)—from tert-butyl (3RS,4RS)-4-[4-(3-benzyloxy-propoxy)-phenyl]-3-(1,2,3,4-tetrahydro-quinolin-7-ylmethoxy)-piperidine-1-carboxylate, (3RS,4RS)-4-[4-(3-benzyloxy-propoxy)-phenyl]-3-(1,2,3,4-tetrahydro-quinolin-7-ylmethoxy)-piperidine as a light yellow resin; MS: 487 (M+H)⁺;

3)—from (3RS,4RS)-7-[4-[4-(3-benzyloxy-propoxy)-phenyl]-1-tert-butoxycarbonyl-piperidin-3-yloxymethyl]-1-methyl-quinolinium iodide, (3RS,4RS)-7-[4-[4-(3-benzyloxy-propoxy)-phenyl]-piperidin-3-yloxymethyl]-1-methyl-quinolinium chloride as a colorless solid; MS: 497 (M)⁺;

4)—from tert-butyl (3RS,4RS)-4-[4-(3-benzyloxy-propoxy)-phenyl]-3-(1-methyl-1,2,3,4-tetrahydro-quinolin-7-ylmethoxy)-piperidine-1-carboxylate, (3RS,4RS)-4-[4-(3-benzyloxy-propoxy)-phenyl]-3-(1-methyl-1,2,3,4-tetrahydro-quinolin-7-ylmethoxy)-piperidine as a light yellow oil; MS: 501 (M+H)⁺;

5)—from tert-butyl (3RS,4RS)-4-[4-(3-benzyloxy-propoxy)-phenyl]-3-(isoquinolin-6-ylmethoxy)-piperidine-1-carboxylate, (3RS,4RS)-4-[4-(3-benzyloxy-propoxy)-phenyl]-3-(isoquinolin-6-ylmethoxy)-piperidine as a light yellow resin; MS: 483 (M+H)⁺;

6)—from tert-butyl (3RS,4RS)-4-[4-(3-benzyloxy-propoxy)-phenyl]-3-(2-methyl-1,2,3,4-tetrahydro-isoquinolin-6-yl-methoxy)-piperidine-1-carboxylate, (3RS,4RS)-4-[4-(3-benzyloxy-propoxy)-phenyl]-3-(2-methyl-1,2,3,4-tetrahydro-isoquinolin-6-ylmethoxy)-piperidine as a light yellow oil; MS: 501 (M+H)⁺;

7)—from tert-butyl (3RS,4RS)-4-[4-[3-(2-methoxy-benzyloxy)-propoxy]-phenyl]-3-(quinolin-7-ylmethoxy)-piperidine-1-carboxylate, (3RS,4RS)-4-[4-[3-(2-methoxy-benzyloxy)-propoxy)-phenyl]-3-(quinolin-7-ylmethoxy)-piperidine as a light yellow resin; MS: 513 (M+H)⁺;

8)—from tert-butyl (3RS,4RS)-3-(1H-benzimidazol-5-ylmethoxy)-4-[4-(3-benzyloxy-propoxy)-phenyl]-piperidine-1-carboxylate with hydrogen chloride in methanol, (3RS,4RS)-3-(1H-benzimidazol-5-ylmethoxy)-4-[4-(3-benzyloxy-propoxy)-phenyl]-piperidine as a colorless resin; MS: 472 (M+H)⁺;

9) from tert-butyl (3RS,4RS)-4-[4-[3-(2-methoxy-benzyloxy)-propoxy]-phenyl]-3-(1-oxy-quinolin-7-ylmethoxy)-piperidine-1-carboxylate with hydrogen chloride in methanol, (3RS,4RS)-4-[4-[3-(2-methoxy-benzyloxy)-propoxy]-phenyl]-3-(1-oxy-quinolin-7-ylmethoxy)-piperidine as a colorless solid; MS: 529 (M+H)⁺;

10) from tert-butyl (3RS,4RS)-4-[4-[3-(2-methoxy-benzyloxy)-propoxy]-phenyl]-3-(2-oxo-1,2-dihydro-quinolin-7-ylmethoxy)-piperidine-1-carboxylate with zinc bromide in methylene chloride, (3RS,4RS)-4-[4-[3-(2-methoxy-benzyloxy)-propoxy]-phenyl]-3-(2-oxo-1,2-dihydro-quinolin-7-ylmethoxy)-piperidine as a colorless solid; MS: 529-(M+H)⁺;

11) from tert-butyl (3RS,4RS)-3-(isoquinolin-7-ylmethoxy)-4-[4-[3-(2-methoxy-benzyloxy)-propoxy]-phenyl]-piperidine-1-carboxylate with hydrogen chloride in methanol, (3RS,4RS)-3-(isoquinolin-7-ylmethoxy)-4-[4-[3-(2-methoxy-benzyloxy)-propoxy]-phenyl]-piperidine as a light yellow oil; MS: 513 (M+H)⁺;

12)—from tert-butyl (3RS,4RS)-4-[4-[3-(2-methoxy-benzyloxy)-propoxy]-phenyl]-3-(1,2,3,4-tetrahydro-quinolin-7-ylmethoxy)-piperidine-1-carboxylate with hydrogen chloride in methanol, (3RS,4RS)-4-[4-[3-(2-methoxy-benzyloxy)-propoxy]-phenyl]-3-(1,2,3,4-tetrahydro-quinolin-7-ylmethoxy)-piperidine as a pale yellow syrup; MS: 517 (M+H)⁺.

13)—from (3RS,4RS)-4-[4-[3-(2-methoxy-benzyloxy)-propoxy]-phenyl]-3-(quinoxalin-6-ylmethoxy)-piperidin-1-carboxylic acid tert-butyl ester with zinc bromide in methylene chloride, (3RS,4RS)-4-[4-[3-(2-methoxy-benzyloxy)-propoxy]-phenyl]-3-(quinoxalin-6-ylmethoxy)-piperidine as a yellow oil; MS:514 (M+H)⁺.

The BOC derivatives used as starting materials were prepared in an analogous manner to that described in Examples 1 and 5:

(a) By alkylating tert-butyl (3RS,4RS)-4-[4-(3-benzyloxy-propoxy)-phenyl]-3-hydroxy-piperidine-1-carboxylate [Example 86 (n)] with 7-bromomethyl-quinoline hydrobromide [J. Am. Chem. Soc. 77, 1054 (1955)] analogously to Example 1 (g) there was obtained tert-butyl (3RS,4RS)-4-[4-(3-benzyloxy-propoxy)-phenyl]-3-(quinolin-7-ylmethoxy)-piperidine-1-carboxylate as a colorless oil; MS: 583 (M+H)⁺.

(b) A solution of 116 mg (0.20 mmol) of tert-butyl (3RS,4RS)-4-[4-(3-benzyloxy-propoxy)-phenyl]-3-(quinolin-7-ylmethoxy)-piperidine-1-carboxylate in 1.5 ml of methanol was treated with 24 mg (0.1 mmol) of nickel(II) chloride hexahydrate and 30 mg (0.8 mmol) of sodium borohydride. The dark suspension was stirred at 0° C. for 1 hour and at room temperature for a further hour. For the working-up, the mixture was partitioned between 20 ml of ether and 5 ml of aqueous saturated ammonium chloride solution and then the organic phase was separated. The aqueous phase was extracted three times with 20 ml of ether each time. The combined ether phases were dried over magnesium sulfate and finally the solvent was distilled off under reduced pressure. The crude product (150 mg) was purified by chromatography on silica gel with a 1:1 mixture of ethyl acetate and hexane as the eluent. There were obtained 70 mg (60% of theory) of tert-butyl (3RS,4RS)-4-[4-(3-benzyloxy-propoxy)-phenyl]-3-(1,2,3,4-tetrahydro-quinolin-7-ylmethoxy)-piperidine-1-carboxylate as a light yellow resin; MS: 587 (M+H)⁺.

(c) A solution of 146 mg (0.25 mmol) of tert-butyl (3RS,4RS)-4-[4-(3-benzyloxy-propoxy)-phenyl]-3-(quinolin-7-ylmethoxy)-piperidine-1-carboxylate in 0.5 ml of absolute chloroform was treated with 40 .mu.1 (0.6 mmol) of methyl iodide and heated to reflux for 3 hours. Subsequently, a further 40 .mu.1 of methyl iodide were added and the mixture was heated at reflux temperature overnight. The solvent was distilled off under reduced pressure and there was obtained crude (3RS,4RS)-7-[4-[4-(3-benzyloxy-propoxy)-phenyl]-1-tert-butoxycarbonyl-piperidin-3-yloxymethyl]-1-methyl-quinolinium iodide, which was used in the next step without characterization.

(d) A solution of 91 mg (0.125 mmol) of crude (3RS,4RS)-7-[4-[4-(3-benzyloxy-propoxy)-phenyl]-1-tert-butoxycarbonyl-piperidin-3-yloxymethyl]-1-methyl-quinolinium iodide in 1 ml of methanol was treated at 0° C. with 47 mg (0.125 mmol) of sodium borohydride, then warmed to room temperature and stirred at room temperature for 2 hours. For the working-up, the mixture was partitioned between 50 ml of ethyl acetate and 50 ml of aqueous 5% sodium hydrogen carbonate solution and thereafter the organic phase was separated. The aqueous phase was extracted three times with 25 ml of ethyl acetate each time. The combined ethyl acetate phases were dried over magnesium sulfate and finally the solvent was distilled off under reduced pressure. The crude tert-butyl (3RS,4RS)-4-[4-(3-benzyloxy-propoxy)-phenyl]-3-(1-methyl-1,2,3,4-tetrahydro-quinolin-7-ylmethoxy)-piperidine-1-carboxylate was used in the next step without characterization.

(e) By alkylating tert-butyl (3RS,4RS)-4-[4-(3-benzyloxy-propoxy)-phenyl]-3-hydroxy-piperidine-1-carboxylate [Example 86 (n)] with 6-bromomethyl-isoquinoline hydrobromide (Example 4) analogously to Example 1 (g) there was obtained tert-butyl (3RS,4RS)-4-[4-(3-benzyloxy-propoxy)-phenyl]-3-(isoquinolin-6-ylmethoxy)-piperidine-1-carboxylate as a colorless oil; MS: 583 (M+H)⁺.

(f) In an analogous manner to that previously described, by alkylating tert-butyl (3RS,4RS)-4-[4-(3-benzyloxy-propoxy)-phenyl]-3-(isoquinolin-6-ylmethoxy)-piperidine-1-carboxylate with methyl iodide in chloroform and subsequently reducing with sodium borohydride in methanol there was obtained tert-butyl (3RS,4RS)-4-[4-(3-benzyloxy-propoxy)-phenyl]-3-(2-methyl-1,2,3,4-tetrahydro-isoquinolin-6-ylmethoxy)-piperidine-1-carboxylate, which was used in the next step as the crude product without further characterization.

(g) (α) A solution of 5.2 g (17.7 mmol) of tert-butyl (3RS,4RS)-3-hydroxy-4-(4-hydroxy-phenyl)-piperidine-1-carboxylate [Example 46 (b)] and 3.37 ml (3.8 g, 17.7 mmol) of 2-methoxybenzyl 3-chloropropyl ether in 18 ml of absolute DMF was treated with 3.7 g (26.9 mmol) of anhydrous potassium carbonate and stirred at 120° C. for 60 h. For the working-up, the reaction mixture was partitioned between 250 ml of water and 250 ml of ethyl acetate. The organic phase was separated and the aqueous phase was extracted three times with 100 ml of ethyl acetate each time. The combined organic phases were washed twice with 100 ml of water each time and finally the solvent was distilled off under reduced pressure. The crude product was crystallized by treatment with ether. There were obtained 7.3 g (88% of theory) of tert-butyl (3RS,4RS)-3-hydroxy-4-[4-[3-(2-methoxy-benzyloxy)-propoxy]-phenyl]-piperidine-1-carboxylate as a colorless solid; MS: 472 (M+H)⁺.

(β) Subsequent alkylation of tert-butyl (3RS,4RS)-3-hydroxy-4-[4-[3-(2-methoxy-benzyloxy)-propoxy]-phenyl]-piperidine-1-carboxylate with 7-bromomethyl-quinoline hydrobromide ([J. Am. Chem. Soc. 77, 1054 (1955)] analogously to Example 1 (g) gave tert-butyl (3RS,4RS)-4-[4-[3-(2-methoxy-benzyloxy)-propoxy]-phenyl]-3-(quinolin-7-ylmethoxy)-piperidine-1-carboxylate as a light yellow resin; MS: 613 (M+H)⁺.

The 2-methoxybenzyl 3-chloropropyl ether used as the alkylating agent was prepared as follows:

A solution of 24.6 g (0.157 mmol) of 2-methoxybenzyl chloride and 26 ml (29.4 g, 0.311 mmol) of 3-chloro-1-propanol in 150 ml of absolute DMF was treated portionwise at 10° C. within 2.5 hours with 8.4 g (0.196 mmol) of sodium hydride dispersion (55% in refined oil) and stirred at room temperature for 1 hour. Subsequently, a further 1.0 g (0.023 mmol) of sodium hydride dispersion was added at room temperature and the mixture was stirred for a further 3 hours. For the working-up, the reaction mixture was partitioned between 500 ml of water and 500 ml of ethyl acetate. The organic phase was separated. The aqueous phase was extracted four times with 250 ml of ethyl acetate each time. The combined organic phases were washed twice with 250 ml of water each time and finally the solvent was distilled off under reduced pressure. The crude product (44 g) was purified by chromatography on silica gel with a 1:2 mixture of methylene chloride and hexane as the eluent. There were obtained 25.0 g (74% of theory) of 2-methoxybenzyl 3-chloropropyl ether as a colorless oil; MS: 214. 216 (M)⁺.

(h) (α) A solution of 2.15 g (14.50 mmol) of (1H-benzimidazol-5-yl)-methanol (DE 2,813,523] in 55 ml of absolute DMF was treated with 4.01 g of anhydrous potassium carbonate and dropwise with 3.15 ml (2.96 g, 16.02 mmol) of SEM chloride. After 3 hours at room temperature the reaction mixture was filtered and the majority of the DMF was distilled off under a high vacuum. For the working-up, the residue was partitioned between 60 ml of ethyl acetate and 60 ml of water and thereafter the organic phase was separated. The aqueous phase was extracted twice with 60 ml of ethyl acetate each time. The combined organic phases were dried over magnesium sulfate and finally the solvent was distilled off under reduced pressure. The crude product (4.57 g) was purified by chromatography on silica gel with a 14:1:0.1 mixture of methylene chloride, methanol and 28% ammonia solution as the eluent. There were obtained 1.26 g (31% of theory) of a 1:2 or 2:1 mixture of [3-(2-trimethylsilyl-ethoxymethyl)-3H-benzimidazol-5-yl]-methanol and [1-(2-trimethylsilyl-ethoxy-methyl)-1H-benzimidazol-5-yl]-methanol as an orange colored oil; MS: 278 (M)⁺.

(β) Chlorination of a 1:2 or 2:1 mixture of [3-(2-trimethylsilyl-ethoxymethyl)-3H-benzimidazol-5-yl]-methanol and [1-(2-trimethylsilyl-ethoxymethyl)-1H-benzimidazol-5-yl]-methanol was effected in an analogous manner to that described in Example 5 (c) and yielded a 1:2 or 2:1 mixture of 6-chloromethyl-1-(2-trimethylsilyl-ethoxymethyl)-1H-benzimidazole and 5-chloromethyl-1-(2-trimethylsilyl-ethoxymethyl)-1H-benzimidazole as a light yellow oil; MS: 296, 298 (M)⁺.

(γ) Alkylation of tert-butyl (3RS,4RS)-4-[4-(3-benzyloxy-propoxy)-phenyl]-3-hydroxy-piperidine-1-carboxylate [Example 86 (n)] with a 1:2 or 2:1 mixture of 6-chloromethyl-1-(2-trimethylsilyl-ethoxymethyl)-1H-benzimidazole and 5-chloromethyl-1-(2-trimethylsilyl-ethoxymethyl)-1H-benzimidazole yielded a 2:1 or 1:2 mixture of tert-butyl (3RS,4RS)-4-[4-(3-benzyloxy-propoxy)-phenyl]-3-[1-(2-trimethylsilyl-ethoxymethyl)-1H-benzimidazol-5-ylmethoxy]-piperidine-1-carboxylate and tert-butyl (3RS,4RS)-4-[4-(3-benzyloxy-propoxy)-phenyl]-3-[3-(2-trimethylsilanyl-ethoxymethyl)-3H-benzimidazol-5-ylmethoxy]-piperidine-1-carboxylate as a yellow oil; MS: 702 (M+H)⁺.

(δ) A solution of 328 mg (0.467 mmol) of a 2:1 or 1:2 mixture of (3RS,4RS)-4-[4-(3-benzyloxy-propoxy)-phenyl]-3-[1-(2-trimethylsilyl-ethoxy methyl)-1H-benzimidazol-5-ylmethoxy]-piperidine-1-carboxylate and tert-butyl (3RS,4RS)-4-[4-(3-benzyloxy-propoxy)-phenyl]-3-[3-(2-trimethylsilanyl-ethoxy-methyl)-3H-benzimidazol-5-ylmethoxy]-piperidine-1-carboxylate in 14 ml of absolute tetrahydrofuran was treated with 3.5 ml of a 1.1M tetrabutylammonium fluoride solution in tetrahydrofuran and heated at reflux temperature for 2 hours. For the working-up, the reaction mixture was partitioned between 50 ml of ethyl acetate and 50 ml of sodium hydrogen carbonate solution (5%) and subsequently the organic phase was separated. The aqueous phase was extracted twice with 50 ml of ethyl acetate each time. The combined organic phases were washed with 25 ml of water each time, dried over magnesium sulfate and finally the solvent was distilled off under reduced pressure. The crude product (280 mg) was purified by chromatography on silica gel with a 14:1:0.1 mixture of methylene chloride, methanol and 28% ammonia solution as the eluent. There were obtained 176 mg (66% of theory) of tert-butyl 3RS,4RS)-3-(1H-benzimidazol-5-ylmethoxy)-4-[4-(3-benzyloxy-propoxy)-phenyl]-piperidine-1-carboxylate as a yellow oil; MS: 572 (M+H)⁺.

(i) A solution of 240 mg (about 0.82 mmol) of 60-70% 3-chloroperbenzic acid in 15 ml of chloroform was added dropwise at 0° C. to a solution of 459 mg (0.75 mmol) of tert-butyl (3RS,4RS)-4-[4-[3-(2-methoxy-benzyloxy)-propoxy]-phenyl]-3-(quinolin-7-ylmethoxy)-piperidine-1-carboxylate in 30 ml of chloroform. The reaction mixture was stirred at room temperature for 2.5 hours and then, for the working-up, partitioned between 50 ml of chloroform and 50 ml of 10% potassium carbonate solution. The organic phase was separated and the aqueous phase was extracted three times with 25 ml of chloroform each time. The combined organic phases were washed twice with 25 ml of water each time, dried over magnesium sulfate and finally the solvent was distilled off under reduced pressure. The crude product was purified by chromatography on silica gel with a 19:1 mixture of methylene chloride and methanol as the eluent. There were obtained 450 mg (96% of theory) of tert-butyl (3RS,4RS)-4-[4-[3-(2-methoxy-benzyloxy)-propoxy]-phenyl]-3-(1-oxy-quinolin-7-ylmethoxy)-piperidine-1-carboxylate as a light yellow resin; MS: 629 (M+H)⁺.

(j) A solution of 50 mg (0.080 mmol) of tert-butyl (3RS,4RS)-4-[4-[3-(2-methoxy-benzyloxy)-propoxy]-phenyl]-3-(1-oxy-quinolin-7-ylmethoxy)-piperidine-1-carboxylate in 0.5 ml of chloroform was treated with 17 mg (0.088 mmol) of tosyl chloride and 0.5 ml of 10% potassium carbonate solution and stirred intensively at room temperature for 3 hours. For the working-up, the reaction mixture was partitioned between 20 ml of ethyl acetate and 20 ml of water, the organic phase was then separated and the aqueous phase was extracted three times with 20 ml of ethyl acetate each time. The combined organic phases were washed twice with 25 ml of water each time, dried over magnesium sulfate and finally the solvent was distilled off under reduced pressure. The crude product (60 mg) was purified by chromatography on silica gel with a 2:1 mixture of ethyl acetate and hexane as the eluent. There were obtained 37 mg (74% of theory) of tert-butyl (3RS,4RS)-4-[4-[3-(2-methoxy-benzyloxy)-propoxy]-phenyl]-3-(2-oxo-1,2-dihydro-quinolin-7-ylmethoxy)-piperidine-1-carboxylate as a colorless solid, which was used directly in the next step without characterization.

(k) In an analogous manner to that described in Example 1 (g), by alkylating tert-butyl (3RS,4RS)-3-hydroxy-4-[4-[3-(2-methoxy-benzyloxy)-propoxy]-phenyl]-piperidine-1-carboxylate [Example 120 (g) (a)] with 7-bromomethyl-isoquinoline (WO 9,319,059) there was obtained tert-butyl (3RS,4RS)-3-(isoquinolin-7-ylmethoxy)-4-[4-[3-(2-methoxy-benzyloxy)-propoxy]-phenyl]-piperidine-1-carboxylate as a light yellow resin; MS: 613 (M+H)⁺.

(l) In an analogous manner to that described in Example 120 (b), by reducing tert-butyl (3RS,4RS)-4-[4-3-(2-methoxy-benzyloxy)-propoxy]-phenyl]-3-(quinolin-7-ylmethoxy)-piperidine-1-carboxylate [Example 120 (g) (β)] by means of nickel(II) chloride hexahydrate and sodium borohydride there was obtained tert-butyl (3RS,4RS)-4-[4-[3-(2-methoxy-benzyloxy)-propoxy]-phenyl]-3-(1,2,3,4-tetrahydro-quinolin-7-ylmethoxy)-piperidine-1-carboxylate as a pale yellow syrup, which was used in the following step without further purification and characterization.

(m) In analogy to Example 1 (g) by alkylating (3RS,4RS)-3-hydroxy-4-[4-[3-(2-methoxy-benzyloxy)-propoxy]-phenyl]-piperidin-1-carboxylic acid tert-butyl esters [Example 120 (g) (a)] with 6-bromomethyl-quinoxalin [J. Heterocycl. Chem. 11, 595 (1974)] there was obtained (3RS,4RS)-4-[4-[3-(2-methoxy-benzyloxy)-propoxy]-phenyl]-3-(quinoxalin-6-ylmethoxy)-piperidin-1-carboylic acid tert-butyl ester as a pale yellow oil; MS:614 (M+H)⁺.

### Example 121

The following compounds were obtained by cleavage of the BOC group:

1)—From a mixture of tert-butyl (3RS,4RS)- and (3SR,4SR)-4-[4-[(RS)-3-benzyloxy-2-methoxy-propoxy]-phenyl]-3-(1-methoxy-naphthalen-2-ylmethoxy)-piperidine-1-carboxylate by means of zinc bromide in methylene chloride, a mixture of (3RS,4RS)-4-[4-[(RS)- and [(SR)-3-benzyloxy-2-methoxy-propoxy]-phenyl]-3-(1-methoxy-naphthalen-2-ylmethoxy)-piperidine as a yellow oil; MS: 542 (M+H)⁺;

2)—from a mixture of tert-butyl (3RS,4RS)- and (3SR,4SR)-4-[4-[(RS)-2-benzyloxy-3-phenoxy-propoxy]-phenyl]-3-(naphthalen-2-ylmethoxy)-piperidine-1-carboxylate by means of trifluoroacetic acid in methylene chloride, a mixture of (3RS,4RS)-4-[4-[(RS)- and -[(SR)-2-benzyloxy-3-phenoxy-propoxy]-phenyl]-3-(naphthalen-2-ylmethoxy)-piperidine trifluoroacetate as a white solid; MS: 574 (M+H)⁺;

3)—from a mixture of tert-butyl (3RS,4RS)- and (3SR,4SR)-4-[4-[(RS)-2-hydroxy-3-(4-methyl-phenylsulfonylamino)-propoxy]-phenyl]-3-(naphthalen-2-ylmethoxy)-piperidine-1-carboxylate by means of trifluoroacetic acid in methylene chloride, a mixture of N-[(RS)- and -(SR)-2-hydroxy-3-[(3RS,4RS)-4-[3-(naphthalen-2-ylmethoxy-piperidin-4-yl]-phenoxy]-propyl]-4-methyl-benzenesulfonamide trifluoroacetate as a white solid; MS: 561 (M+H)⁺;

4)—from a mixture of tert-butyl (3RS,4RS)- and (3SR,4SR)-4-[4-[(RS)-2-allyloxy-4-phenyl-butoxy]-phenyl]-3-(naphthalen-2-ylmethoxy)-piperidine-1-carboxylate by means of trifluoroacetic acid in methylene chloride, a mixture of (3RS,4RS)-4-[4-[(RS)- and -[(SR)-2-allyloxy-4-phenyl-butoxy]-phenyl]-3-(naphthalen-2-ylmethoxy)-piperidine trifluoroacetate as a white solid; MS: 522 (M+H)⁺;

The BOC derivatives used as starting materials were prepared as follows:

(a) In an analogous manner to that described in Example 1 (g), by alkylating a mixture of tert-butyl (3RS,4RS)- and (3SR,4SR)-3-hydroxy-4-[(RS)-4-oxiranylmethoxy-phenyl]-piperidine-1-carboxylate [Example 86 (d)] with 1-methoxy-2-bromomethyl-naphthalene [Example 7 (f)] there was obtained a mixture of tert-butyl (3RS,4RS)- and (3SR,4SR)-3-(1-methoxy-naphthalen-2-ylmethoxy)-4-[(RS)-4-oxiranylmethoxy-phenyl]-piperidine-1-carboxylate. Subsequent epoxide opening with sodium benzylate in N,N-dimethylformamide yielded a mixture of tert-butyl (3RS,4RS)- and (3SR,4SR)-4-[4-[(RS)-3-benzyloxy-2-hydroxy-propoxy)-phenyl]-3-(1-methoxy-naphthalen-2-ylmethoxy)-piperidine-1-carboxylate, alkylation of which with methyl iodide analogously to Example 1 (g) gave a mixture of tert-butyl (3RS,4RS)- and (3SR,4SR)-4-[4-[(RS)-3-benzyloxy-2-methoxy-propoxy)-phenyl]-3-(1-methoxy-naphthalen-2-ylmethoxy)-piperidine-1-carboxylate as a colorless oil; MS: 628 (M+H)⁺;

(b) In an analogous manner to that described in Example 1 (g), by alkylating a mixture of tert-butyl (3RS,4RS)- and (3SR,4SR)-4-[4-[(RS)-2-hydroxy-3-phenoxy-propoxy]-phenyl]-3-(naphthalen-2-ylmethoxy)-piperidine-1-carboxylate [Example 86 (d)] with benzyl bromide there was obtained a mixture of tert-butyl (3RS,4RS)- and (3SR,4SR)-4-[4-[(RS)-2-benzyloxy-3-phenoxy-propoxy]-phenyl]-3-(naphthalen-2-ylmethoxy)-piperidine-1-carboxylate as a solid; MS: 674 (M+H)⁺;

(c) Epoxide opening of a mixture of tert-butyl (3RS,4RS)- and (3SR,4SR)-3-(naphthalen-2-ylmethoxy)-4-[(RS)-4-oxiranylmethoxy-phenyl]-piperidine-1-carboxylate [Example 86 (d)] with potassium toluene-4-sulfonamide in analogy to Example 71 (a) yielded a mixture of tert-butyl (3RS,4RS)- and (3SR,4SR)-4-[4-[(RS)-2-hydroxy-3-(4-methyl-phenylsulfonylamino)-propoxy]-phenyl]-3-(naphthalen-2-ylmethoxy)-piperidine-1-carboxylate as a white solid; MS: 661 (M+H)⁺;

(d) Epoxide opening of a mixture of tert-butyl (3RS,4RS)- and (3SR,4SR)-3-(naphthalen-2-ylmethoxy)-4-[(RS)-4-oxiranylmethoxy-phenyl]-piperidine-1-carboxylate [Example 86 (d)] with benzylmagnesium chloride in tetrahydrofuran yielded a mixture of tert-butyl (3RS,4RS)- and (3SR,4SR)-4-[4-[(RS)-2-hydroxy-4-phenyl-butoxy]-phenyl]-3-(naphthalen-2-yl methoxy)-piperidine-1-carboxylate, alkylation of which with allyl bromide analogously to Example 1 (g) gave a mixture of tert-butyl (3RS,4RS)- and (3SR,4SR)-4-[4-[(RS)-2-allyloxy-4-phenyl-butoxy]-phenyl]-3-(naphthalen-2-ylmethoxy)-piperidine-1-carboxylate as a colorless oil; MS: 621 (M)⁺.

### Example 122

The following compounds were obtained by cleavage of the BOC group:

1)—From tert-butyl (3RS,4RS)-3-(naphthalen-2-ylmethoxy)-4-[4-(3-phenyl-propoxy)-phenyl]-piperidine-1-carboxylate by means of trifluoroacetic acid in methylene chloride, (3RS,4RS)-3-(naphthalen-2-ylmethoxy)-4-[4-(3-phenyl-propoxy)-phenyl]-piperidine trifluoroacetate as a colorless solid; MS: 452 (M+H)⁺;

2)—from tert-butyl (3RS,4RS)-3-(naphthalen-2-ylmethoxy)-4-[4-(4-phenyl-butoxy)-phenyl]-piperidine-1-carboxylate by means of trifluoroacetic acid in methylene chloride, (3RS,4RS)-3-(naphthalen-2-ylmethoxy)-4-[4-(4-phenyl-butoxy)-phenyl]-piperidine trifluoroacetate as a colorless solid; MS: 466 (M+H)⁺;

3)—from tert-butyl (3RS,4RS)-3-(naphthalen-2-ylmethoxy)-4-[4-(5-phenyl-pentyloxy) phenyl]-piperidine-1-carboxylate by means of trifluoroacetic acid in methylene chloride, (3RS,4RS)-3-(naphthalen-2-ylmethoxy)-4-[4-(5-phenyl-pentyloxy)-phenyl]-piperidine trifluoroacetate as a colorless solid; MS: 480 (M+H)⁺;

4)—from tert-butyl (E)-(3RS,4RS)-3-(naphthalen-2-ylmethoxy)-4-[4-(3-phenyl-allyloxy)-phenyl]-piperidine-1-carboxylate by means of zinc bromide in methylene chloride, (E)-(3RS,4RS)-3-(naphthalen-2-ylmethoxy)-4-[4-(3-phenyl-allyloxy)-phenyl]-piperidine as a colorless solid; MS: 450 (M+H)⁺;

5)—from tert-butyl (E)-(3RS,4RS)-3-(1,4-dimethoxy-naphthalen-2-ylmethoxy)-4-[4-(3-phenyl-ally loxy)-phenyl]-piperidine-1-carboxylate by means of zinc bromide in methylene chloride, (E)-(3RS,4RS)-3-(1,4-dimethoxy-naphthalen-2-yl-methoxy)-4-[4-(3-phenyl-allyloxy)-phenyl]-piperidine as a beige solid; MS: 510 (M+H)⁺;

6)—from tert-butyl (E)-(3RS,4RS)-3-(4-methylsulfanyl-benzyloxy)-4-[4-(3-phenyl-allyloxy)-phenyl]-piperidine-1-carboxylate by means of zinc bromide in methylene chloride, (E)-(3RS,4RS)-3-(4-methylsulfanyl-benzyloxy)-4-[4-(3-phenyl-allyloxy)-phenyl]-piperidine as a yellowish oil; MS: 446 (M+H)⁺;

7)—from tert-butyl (E)-(3RS,4RS)-3-(1,4-dimethoxy-naphthalen-2-ylmethoxy)-4-[4-(4-phenyl-but-3-enyloxy)-phenyl]-piperidine-1-carboxylate by means of zinc bromide in methylene chloride, (E)-(3RS,4RS)-3-(1,4-dimethoxy-naphthalen-2-ylmethoxy)-4-[4-(4-phenyl-but-3-enyloxy)-phenyl]-piperidine as a beige solid; MS: 524 (M+H)⁺;

8)—from tert-butyl (3RS,4RS)-4-[4-(3-cyano-benzyloxy)-phenyl}-3-[1-(2-methoxy-ethoxy)-naphthalen-2-ylmethoxy]-piperidine-1-carboxylate by means of zinc bromide in methylene chloride, (3RS,4RS)-3-[4-[3-[1-(2-methoxy-ethoxy)-naphthalen-2-ylmethoxy]-piperidin-4-yl]-phenoxymethyl]-benzonitrile as a viscous, pale yellow oil; MS: 523 (M+H)⁺;

9)—from tert-butyl (3RS,4RS)-3-(1,4-dimethoxy-naphthalen-2-ylmethoxy)-4-[4-(4-phenyl-butoxy)-phenyl]-piperidine-1-carboxylate by means of zinc bromide, (3RS,4RS)-3-(1,4-dimethoxy-naphthalen-2-ylmethoxy)-4-[4-(4-phenyl-butoxy)-phenyl]-piperidine as a beige solid; MS: 526 (M+H)⁺.

The BOC derivatives used as starting materials were prepared as follows:

(a) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 1-bromo-3-phenyl-propane in the presence of potassium carbonate, tert-butyl (3RS,4RS)-3-hydroxy-4-[4-(3-phenyl-propoxy)-phenyl]-piperidine-1-carboxylate, alkylation of which analogously to Example 1 (g) with 2-bromomethyl-naphthalene yielded tert-butyl (3RS,4RS)-3-(naphthalen-2-ylmethoxy)-4-[4-(3-phenyl-propoxy)-phenyl]-piperidine-1-carboxylate as a colorless solid; MS: 551 (M)⁺.

(b) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 4-phenyl-butanol mesylate, prepared according to a generally known procedure, in the presence of potassium carbonate there was obtained tert-butyl (3RS,4RS)-3-hydroxy-4-[4-(4-phenyl-butoxy)-phenyl]-piperidine-1-carboxylate, alkylation of which analogously to Example 1 (g) with 2-bromomethyl-naphthalene yielded tert-butyl (3RS,4RS)-3-(naphthalen-2-ylmethoxy)-4-[4-(4-phenyl-butoxy)-phenyl]-piperidine-1-carboxylate as a colorless oil; MS: 566 (M+H)⁺.

(c) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 5-phenyl-pentanol mesylate, prepared according to a generally known procedure, in the presence of potassium carbonate in DMF there was obtained tert-butyl (3RS,4RS)-3-hydroxy-4-[4-(3-phenyl-propoxy)-phenyl]-piperidine-1-carboxylate, alkylation of which analogously to Example 1 (g) with 2-bromomethyl-naphthalene yielded tert-butyl (3RS,4RS)-3-(naphthalen-2-ylmethoxy)-4-[4-(5-phenyl-pentyloxy)-phenyl]-piperidine-1-carboxylate as a colorless solid; MS: 580 (M+H)⁺.

(d) In an analogous manner to that described in Example 44 (e), by alkylating tert-butyl (3RS,4RS)-3-hydroxy-4-(4-hydroxy-phenyl)-piperidine-1-carboxylate [Example 46 (b)] with cinnamyl bromide in the presence of potassium carbonate in acetone there was obtained tert-butyl (E)-(3RS,4RS)-3-hydroxy-4-[4-(3-phenyl-allyloxy)-phenyl]-piperidine-1-carboxylate, alkylation of which analogously to Example 1 (g) with 2-bromomethyl-naphthalene yielded tert-butyl (E)-(3RS,4RS)-3-(naphthalen-2-ylmethoxy)-4-[4-(3-phenyl-allyloxy)-phenyl]-piperidine-1-carboxylate as a colorless solid; MS: 550 (M+H)⁺.

(e) Alkylation of tert-butyl (E)-(3RS,4RS)-3-hydroxy-4-[4-(3-phenyl-allyloxy)-phenyl]-piperidine-1-carboxylate analogously to Example 1 (g) with 2-chloromethyl-1,4-dimethoxy-naphthalene [J. Org. Chem. (1983), 48(19), 3265-3268] gave tert-butyl (E)-(3RS,4RS)-3-(1,4-dimethoxy-naphthalen-2-ylmethoxy)-4-[4-(3-phenyl-ally loxy)-phenyl]-piperidine-1-carboxylate as a yellowish solid; MS: 610 (M+H)⁺.

(f) Alkylation of tert-butyl (E)-(3RS,4RS)-3-hydroxy-4-[4-(3-phenyl-allyloxy)-phenyl]-piperidine-1-carboxylate analogously to Example 1 (g) with 4-methylthio-benzyl chloride [J. Org. Chem. (1988), 53(3), 561-569] gave tert-butyl (E)-(3RS,4RS)-3-(4-methylsulfanyl-benzyloxy)-4-[4-(3-phenyl-allyloxy)-phenyl]-piperidine-1-carboxylate as a yellowish solid; MS: 546 (M+H)⁺.

(g) In an analogous manner to that described in Example 44 (e), by alkylating tert-butyl (3RS,4RS)-3-hydroxy-4-(4-hydroxy-phenyl)-piperidine-1-carboxylate [Example 46 (b)] with (E)-4-phenyl-3-buten-1-ol mesylate there was obtained tert-butyl (E)-(3RS,4RS)-3-hydroxy-4-[4-(4-phenyl-but-3-enyloxy)-phenyl]-piperidine-1-carboxylate, alkylation of which analogously to Example 1 (g) with 2-chloromethyl-1,4-dimethoxy-naphthalene yielded (E)-(3RS,4RS)-3-(1,4-dimethoxy-naphthalen-2-ylmethoxy)-4-[4-(4-phenyl-but-3-enyloxy)-phenyl]-piperidine-1-carboxylate as a viscous, pale yellow oil; MS: 624 (M+H)⁺.

The (E)-4-phenyl-but-3-enyl methanesulfonate used as the starting material was prepared as follows:

(α) A solution of 3.24 mg (20 mmol) of (E)-styrylacetic acid in 20 ml of methanol, 2 ml of trimethyl orthoformate and 192 mg (2 mmol) of methanesulfonic acid was stirred at 50° C. under argon for one hour. For the working-up, the mixture was neutralized with 2 mmol of sodium methylate and subsequently the solvent mixture was distilled off under reduced pressure. There was obtained methyl (E)-4-phenyl-but-3-enoate as a colorless liquid in quantitative yield; MS: 176 (M)⁺.

(β) In an analogous manner to that described in Example 5 (b), by reducing methyl (E)-4-phenyl-but-3-enoate by means of lithium aluminium hydride in tetrahydrofuran there was obtained (E)-4-phenyl-but-3-en-1-ol, which was converted into (E)-4-phenyl-but-3-enyl methanesulfonate in analogy to the procedure described in J. Chem. Soc. Perk. Trans. 1 (1988), (6), 1517-1519 for the preparation of the (Z) isomer.

(h) In an analogous manner to that described in Example 1 (g), by alkylating tert-butyl (3RS,4RS)-4-(4-allyloxy-phenyl)-3-hydroxy-piperidine-1-carboxylate [Example 86 (b)] with 2-chloromethyl-1-(2-methoxy-ethoxy)-naphthalene there was obtained (3RS,4RS)-4-(4-allyloxy-phenyl)-3-[1-(2-methoxy-ethoxy)-naphthalen-2-ylmethoxy)-piperidine-1-carboxylate, from which after cleavage of the allyl group by means of bis-(triphenylphosphine)-palladium(II) diacetate analogously to Example 152 (e) there resulted tert-butyl (3RS,4RS)-4-(4-hydroxy-phenyl)-3-[1-(2-methoxy-ethoxy)-naphthalen-2-ylmethoxy]-piperidine-1-carboxylate, alkylation of which analogously to Example 44 (e) with 3-bromomethyl-benzonitrile gave tert-butyl (3RS,4RS)-4-[4-(3-cyano-benzyloxy)-phenyl]-3-[1-(2-methoxy-ethoxy)-naphthalen-2-ylmethoxy]-piperidine-1-carboxylate as a colorless oil; MS: 623 (M+H)⁺.

The 2-chloromethyl-1-(2-methoxy-ethoxy)-naphthalene used as the alkylating agent was obtained as follows:

By alkylating methyl 1-hydroxy-naphthalene-2-carboxylate analogously to Example 1 (g) with 2-bromoethyl methyl ether there was obtained methyl 1-(2-methoxy-ethoxy)-naphthalene-2-carboxylate, which was subsequently converted analogously to Example 5 (b)-(c) firstly into [1-(2-methoxy-ethoxy)-naphthalen-2-yl]-methanol and then into 2-chloromethyl-1-(2-methoxy-ethoxy)-naphthalene, which was finally obtained as a beige solid; MS: 250 (M)⁺.

(i) In an analogous manner to that described in Example 1 (g), by alkylating tert-butyl (3RS,4RS)-3-hydroxy-4-[4-(4-phenyl-butoxy)-phenyl]-piperidine-1-carboxylate [Example 122 (b)] with 2-chloromethyl-1,4-dimethoxy-naphthalene [J. Org. Chem. (1983), 48(19),3265-3268] there was obtained tert-butyl (3RS,4RS)-3-(1,4-dimethoxy-naphthalen-2-ylmethoxy)-4-[4-(4-phenyl-butoxy)-phenyl]-piperidine-1-carboxylate as a colorless, viscous oil; MS: 626 (M+H)⁺.

### Example 123

The following compounds were obtained by cleavage of the BOC group:

1)—From tert-butyl (3RS,4RS)-4-[4-(3-benzyloxy-propoxy)-phenyl]-3-(1,4-dimethoxy-naphthalen-2-ylmethoxy)-piperidine-1-carboxylate by means of zinc bromide in methylene chloride, (3RS,4RS)-4-[4-(3-benzyloxy-propoxy)-phenyl]-3-(1,4-dimethoxy-naphthalen-2-ylmethoxy)-piperidine hydrobromide as a beige solid; MS: 542 (M+H)⁺;

2)—from tert-butyl (3RS,4RS)-3-(1,4-dimethoxy-naphthalen-2-ylmethoxy)-4-{4-[3-(thiophen-2-ylmethoxy)-propoxy]-phenyl}-piperidine-1-carboxylate by means of zinc bromide in methylene chloride, {3RS,4RS}-3-{1,4-dimethoxy-naphthalen-2-ylmethoxy}-4-[4-[3-(thiophen-2-ylmethoxy)-propoxy)-phenyl]-piperidine as a beige solid; MS: 548 (M+H)⁺;

3)—from tert-butyl (3RS,4RS)-3-(4-methylsulfanyl-benzyloxy)-4-[4-[3-(thiophen-2-ylmethoxy)-propoxy]-phenyl]-piperidine-1-carboxylate by means of zinc bromide in methylene chloride, (3RS,4RS)-3-(4-methylsulfanyl-benzyloxy)-4-[4-[3-(thiophen-2-ylmethoxy)-propoxy]-phenyl]-piperidine as a yellowish resin; MS: 484 (M+H)⁺;

4)—from tert-butyl (3RS,4RS)-4-[4-(3-benzylsulfanyl-propoxy)-phenyl]-3-(1,4-dimethoxy-naphthalen-2-ylmethoxy)-piperidine-1-carboxylate by means of zinc bromide in methylene chloride, (3RS,4RS)-4-[4-(3-benzylsulfanyl-propoxy)-phenyl]-3-(1,4-dimethoxy-naphthalen-2-ylmethoxy)-piperidine as a yellow solid; MS: 558 (M+H)⁺;

5)—from tert-butyl (3RS,4RS)-4-[4-(3-benzylsulfanyl-propoxy)-phenyl]-3-(naphthalen-2-ylmethoxy)-piperidine-1-carboxylate by means of trifluoroacetic acid in methylene chloride, (3RS,4RS)-4-[4-(3-benzylsulfanyl-propoxy)-phenyl]-3-(naphthalen-2-ylmethoxy)-piperidine trifluoroacetate as a colorless solid; MS: 498 (M+H)⁺;

6)—from tert-butyl (3RS,4RS)-3-(naphthalen-2-ylmethoxy)-4-[4-(2-phenethyloxy-ethoxy)-phenyl]-piperidine-1-carboxylate by means of trifluoroacetic acid in methylene chloride, (3RS,4RS)-3-(naphthalen-2-ylmethoxy)-4-[4-(2-phenethyloxy-ethoxy)-phenyl]-piperidine trifluoroacetate as a colorless solid; MS: 482 (M+H)⁺;

7)—from tert-butyl (3RS,4RS)-3-(4-methylsulfanyl-benzyl-oxy)-4-[4-(2-phenethyloxy-ethoxy)-phenyl]-piperidine-1-carboxylate by means of zinc bromide in methylene chloride, (3RS,4RS)-3-(4-methyl sulfanyl-benzyloxy)-4-[4-(2-phenethyloxy-ethoxy)-phenyl]-piperidine hydrobromide as a pale yellow solid; MS: 478 (M+H)⁺;

8)—from tert-butyl (3RS,4RS)-3-(1,4-dimethoxy-naphthalen-2-ylmethoxy)-4-[4-(2-phenethyloxy-ethoxy)-phenyl]-piperidine-1-carboxylate by means of zinc bromide in methylene chloride, (3RS,4RS)-3-(1,4-dimethoxy-naphthalen-2-ylmethoxy)-4-[4-(2-phenethyloxy-ethoxy)-phenyl]-piperidine as a colorless solid; MS: 542 (M+H)⁺;

9)—from tert-butyl (3RS,4RS)-3-(naphthalen-2-ylmethoxy)-4-[4-[2-(pyrimidin-2-yloxy)-ethoxy]-phenyl]-piperidine-1-carboxylate by means of trifluoroacetic acid in methylene chloride, (3RS,4RS)-2-(2-[4-[3-(naphthalen-2-ylmethoxy)-piperidin-4-yl]-phenoxy]-ethoxy)-piperidine trifluoroacetate as a colorless solid; MS: 456 (M+H)⁺;

10)—from tert-butyl (3RS,4RS)-3-(naphthalen-2-ylmethoxy)-4-[4-(3-phenoxy-propoxy)-phenyl]-piperidine-1-carboxylate by means of trifluoroacetic acid in methylene chloride, (3RS,4RS)-3-(naphthalen-2-ylmethoxy)-4-[4-(3-phenoxy-propoxy)-phenyl]-piperidine trifluoroacetate as a colorless solid; MS: 468 (M+H)⁺;

11)—from tert-butyl (3RS,4RS)-3-(naphthalen-2-ylmethoxy)-4-[4-(4-phenoxy-butoxy)-phenyl]-piperidine-1-carboxylate by means of trifluoroacetic acid in methylene chloride, (3RS,4RS)-3-(naphthalen-2-ylmethoxy)-4-[4-(4-phenoxy-butoxy)-phenyl]-piperidine trifluoroacetate as a colorless solid; MS: 482 (M+H)⁺;

12)—from tert-butyl (3RS,4RS)-3-(1,4-dimethoxy-naphthalen-2-ylmethoxy)-4-[4-(4-phenoxy-butoxy)-phenyl]-piperidine-1-carboxylate by means of zinc bromide in methylene chloride, (3RS,4RS)-3-(1,4-dimethoxy-naphthalen-2-ylmethoxy)-4-[4-(4-phenoxy-butoxy)-phenyl]-piperidine as a colorless solid; MS: 542 (M+H)⁺;

13)—from tert-butyl (3RS,4RS)-3-(1,4-dimethoxy-naphthalen-2-ylmethoxy)-4-[4-(3-phenylsulfanyl-propoxy)-phenyl]-piperidine-1-carboxylate by means of zinc bromide in methylene chloride, (3RS,4RS)-3-(1,4-dimethoxy-naphthalen-2-ylmethoxy)-4-[4-(3-phenylsulfanyl-propoxy)-phenyl]-piperidine as a brown solid; MS: 544 (M+H)⁺;

14)—from tert-butyl (3RS,4RS)-3-(naphthalen-2-ylmethoxy)-4-[4-(4-phenoxy-but-2-ynyloxy)-phenyl)-piperidine-1-carboxylate by means of zinc bromide in methylene chloride, (3RS,4RS)-3-(naphthalen-2-ylmethoxy)-4-[4-(4-phenoxybut-2-ynyloxy)-phenyl]-piperidine as a yellow, viscous oil; MS: 478 (M+H)⁺;

15)—from tert-butyl (3RS,4RS)-3-(1,4-dimethoxy-naphthalen-2-ylmethoxy)-4-[4-(4-phenoxy-but-2-ynyloxy)-phenyl]-piperidine-1-carboxylate by means of zinc bromide in methylene chloride, (3RS,4RS)-3-(1,4-dimethoxy-naphthalen-2-ylmethoxy)-4-[4-(4-phenoxy-but-2-ynyloxy)-phenyl]-piperidine as a brown solid; MS: 538 (M+H)⁺;

16)—from tert-butyl (E)-(3RS,4RS)-3-(naphthalen-2-ylmethoxy)-4-[4-(4-phenoxy-but-2-enyloxy)-phenyl]-piperidine-1-carboxylate by means of zinc bromide in methylene chloride, (E)-(3RS,4RS)-3-(naphthalen-2-ylmethoxy)-4-[4-(4-phenoxy-but-2-enyloxy)-phenyl]-piperidine as a colorless solid; MS: 480 (M+H)⁺;

17)—from tert-butyl (Z)-(3RS,4RS)-3-(naphthalen-2-ylmethoxy)-4-[4-(4-phenoxy-but-2-enyloxy)-phenyl]-piperidine-1-carboxylate by means of zinc bromide in methylene chloride, (Z)-(3RS,4RS)-3-(naphthalen-2-ylmethoxy)-4-[4-(4-phenoxy-but-2-enyloxy)-phenyl]-piperidine as a pale yellow solid; MS: 480 (M+H)⁺;

18)—from tert-butyl (3RS,4RS)-3-(4,8-dimethoxy-naphthalen-2-ylmethoxy)-4-{4-[3-(thiophen-2-ylmethoxy)-propoxy]-phenyl}-piperidine-1-carboxylate by means of zinc bromide in methylene chloride, (3RS,4RS)-3-(4,8-dimethoxy-naphthalen-2-ylmethoxy)-4-[4-[3-(thiophen-2-ylmethoxy)-propoxy]-phenyl]-piperidine as a beige solid; MS: 548 (M+H)⁺;

19)—from tert-butyl (3RS,4RS)-3-(7-hydroxy-naphthalen-2-ylmethoxy)-4-{4-[3-(thiophen-2-ylmethoxy)-propoxy]-phenyl}-piperidine-1-carboxylate by means of hydrogen chloride in methanol, (3RS,4RS)-7-[4-[4-[3-(thiophen-2-ylmethoxy)-propoxy]-phenyl]-piperidin-3-yloxymethyl]-naphthalen-2-ol as a colorless solid; MS: 504 (M+H)⁺;

20)—from tert-butyl (3RS,4RS)-3-[8-methoxy-4-(3-methoxy-propoxy)-naphthalen-2-ylmethoxy]-4-{4-[3-(thiophen-2-ylmethoxy)-propoxy]-phenyl}-piperidine-1-carboxylate by means of zinc bromide in methylene chloride, (3RS,4RS)-3-[8-methoxy-4-(3-methoxy-propoxy)-naphthalen-2-ylmethoxy]-4-[4-[3-(thiophen-2-ylmethoxy)-propoxy]-phenyl]-piperidine as a beige solid; MS: 606 (M+H)⁺;

21)—from a mixture of tert-butyl (3RS,4RS)- and (3SR,4SR)-3-[7-[(RS)-2,3-dimethoxy-propoxy]-naphthalen-2-ylmethoxy]-4-{4-[3-(thiophen-2-ylmethoxy)-propoxy]-phenyl}-piperidine-1-carboxylate by means of zinc bromide in methylene chloride, a mixture of (3RS,4RS)- and (3SR,4SR)-3-[7-[(RS)-2,3-dimethoxy-propoxy]-naphthalen-2-ylmethoxy]-4-[4-[3-(thiophen-2-ylmethoxy)-propoxy]-phenyl]-piperidine as a yellow, viscous oil; MS: 606 (M+H)⁺;

22)—from a mixture of tert-butyl (3RS,4RS)- and (3SR,4SR)-3-[1-[(RS)-2,3-dimethoxy-propoxy]-naphthalen-2-ylmethoxy]-4-{4-[3-(thiophen-2-ylmethoxy)-propoxy]-phenyl}-piperidine-1-carboxylate by means of zinc bromide in methylene chloride, a mixture of (3RS,4RS)- and (3SR,4SR)-3-[1-[(RS)-2,3-dimethoxy-propoxy]-naphthalen-2-ylmethoxy]-4-[4-[3-(thiophen-2-ylmethoxy)-propoxy]-phenyl]-piperidine as a yellow, viscous oil; MS: 606 (M+H)⁺;

23)—from tert-butyl (3RS,4RS)-3-(1,4-dimethoxy-naphthalen-2-ylmethoxy)-4-{4-[3-(thiophen-3-ylmethoxy)-propoxy]-phenyl}-piperidine-1-carboxylate by means of zinc bromide in methylene chloride, (3RS,4RS)-3-(1,4-dimethoxy-naphthalen-2-ylmethoxy)-4-[4-[3-(thiophen-3-ylmethoxy)-propoxy]-phenyl]-piperidine as a brown solid; MS: 548 (M+H)⁺;

24)—from tert-butyl (3RS,4RS)-3-(7-hydroxy-naphthalen-2-ylmethoxy)-4-{4-[3-(thiophen-3-ylmethoxy)-propoxy]-phenyl}-piperidine-1-carboxylate by means of zinc bromide in methylene chloride, (3RS,4RS)-7-[4-[4-[3-(thiophen-3-ylmethoxy)-propoxy]-phenyl]-piperidin-3-yloxymethyl]-naphthalen-2-ol as a brown solid; MS: 504 (M+H)⁺;

25)—from tert-butyl (3RS,4RS)-3-[8-methoxy-4-(3-methoxy-propoxy)-naphthalen-2-ylmethoxy]-4-{4-[3-(thiophen-3-ylmethoxy)-propoxy]-phenyl}-piperidine-1-carboxylate by means of zinc bromide in methylene chloride, (3RS,4RS)-3-[8-methoxy-4-(3-methoxy-propoxy)-naphthalen-2-ylmethoxy]-4-[4-[3-(thiophen-3-ymethoxy)-propoxy]-phenyl]-piperidine as a yellowish, viscous oil; MS: 606 (M+H)⁺;

26)—from tert-butyl (3RS,4RS)-3-(1,4-dimethoxy-naphthalen-2-ylmethoxy)-4-[4-(3-phenoxy-propoxy)-phenyl]-piperidine-1-carboxylate by means of zinc bromide in methylene chloride, (3RS,4RS)-3-(1,4-dimethoxy-naphthalen-2-ylmethoxy)-4-[4-(3-phenoxy-propoxy)-phenyl]-piperidine as a yellow, viscous oil; MS: 528 (M+H)⁺;

27)—from tert-butyl (3RS,4RS)-3-(1,4-dimethoxy-naphthalen-2-ylmethoxy)-4-{4-[3-(2-methoxy-phenoxy)-propoxy]-phenyl}-piperidine-1-carboxylate by means of zinc bromide in methylene chloride, (3RS,4RS)-3-(1,4-dimethoxy-naphthalen-2-ylmethoxy)-4-[4-[3-(2-methoxy-phenoxy)-propoxy]-phenyl]-piperidine as a pale brown solid; MS: 558 (M+H)⁺;

28)—from tert-butyl (3RS,4RS)-3-(1,4-dimethoxy-naphthalen-2-ylmethoxy)-4-{4-[3-(3-methoxy-phenoxy)-propoxy]-phenyl}-piperidine-1-carboxylate by means of zinc bromide in methylene chloride, (3RS,4RS)-3-(1,4-dimethoxy-naphthalen-2-ylmethoxy)-4-[4-[3-(3-methoxy-phenoxy)-propoxy]-phenyl]-piperidine as a yellow, viscous oil; MS: 558 (M+H)⁺;

29)—from tert-butyl (3RS,4RS)-4-{4-[3-(2-chloro-phenoxy)-propoxy]-phenyl}-3-(1,4-dimethoxy-naphthalen-2-ylmethoxy)-piperidine-1-carboxylate by means of zinc bromide in methylene chloride, (3RS,4RS)-4-[4-[3-(2-chloro-phenoxy)-propoxy]-phenyl]-3-(1,4-dimethoxy-naphthalen-2-ylmethoxy)-piperidine as a yellow, semi-solid product; MS: 562 (M+H)⁺;

30)—from tert-butyl (3RS,4RS)-3-(1,4-dimethoxy-naphthalen-2-ylmethoxy)-4-{4-[3-(2-methoxy-phenylsulfanyl)-propoxy]-phenyl}-piperidine-1-carboxylate by means of zinc bromide in methylene chloride, (3RS,4RS)-3-(1,4-dimethoxy-naphthalen-2-ylmethoxy)-4-[4-[3-(2-methoxy-phenylsulfanyl)-propoxy]-phenyl]-piperidine as a yellow, viscous oil; MS: 574 (M+H)⁺;

31)—from tert-butyl (3RS,4RS)-4-[4-(3-benzylsulfanyl-propoxy)-phenyl]-3-(4,8-dimethoxy-naphthalen-2-ylmethoxy)-piperidine-1-carboxylate by means of zinc bromide in methylene chloride, (3RS,4RS)-4-[4-(3-benzylsulfanyl-propoxy)-phenyl]-3-(4,8-dimethoxy-naphthalen-2-ylmethoxy)-piperidine as a colorless solid; MS: 558 (M+H)⁺;

32)—from tert-butyl (3RS,4RS)-3-(1,4-dimethoxy-naphthalen-2-ylmethoxy)-4-[4-[3-(2-methoxy-benzyloxy)-propoxy]-phenyl}-piperidine-1-carboxylate by means of zinc bromide in methylene chloride, (3RS,4RS)-3-(1,4-dimethoxy-naphthalen-2-ylmethoxy)-4-[4-[3-(2-methoxy-benzyloxy)-propoxy]-phenyl]-piperidine as a yellow syrup; MS: 572 (M+H)⁺;

33)—from a 1:1 mixture of tert-butyl (3R,4R)- and (3S,4S)-3-[7-[(R)-2-hydroxy-3-morpholin-4-yl-propoxy]-naphthalen-2-ylmethoxy]-4-{4-[3-(thiophen-2-ylmethoxy)-propoxy]-phenyl}-piperidine-1-carboxylate by means of hydrogen chloride in methanol, a 1:1 mixture of (R)-1-morpholin-4-yl-3-[(3R,4R)- and -[(3S,4S)-7-[4-[4-[3-(thiophen-2-ylmethoxy)-propoxy]-phenyl]-piperidin-3-yloxymethyl]-naphthalen-2-yloxy]-propan-2-ol dihydrochloride as a beige solid; MS: 647 (M+H)⁺;

34)—from tert-butyl (3RS,4RS)-3-(1,4-dimethoxy-naphthalen-2-ylmethoxy)-4-[4-(2-phenethyloxy-ethoxy)-phenyl]-piperidine-1-carboxylate by means of zinc bromide in methylene chloride, (3RS,4RS)-3-(1,4-dimethoxy-naphthalen-2-ylmethoxy)-4-[4-(2-phenethyloxy-ethoxy)-phenyl]-piperidine, which was oxidized as follows:

A solution of 240 mg of Cer(IV) ammonium nitrate in 1 ml of water was added dropwise at room temperature to a solution of 118 mg of (3RS,4RS)-3-(1,4-dimethoxy-naphthalen-2-ylmethoxy)-4-[4-(2-phenethyloxy-ethoxy)-phenyl]-piperidine in 10 ml of acetonitrile. The reaction solution was stirred at room temperature for 15 minutes and subsequently evaporated under reduced pressure. The residue was partitioned between methylene chloride and water, the organic phase was dried and evaporated under reduced pressure. For purification, the crude product was chromatographed on silica gel using a 9:1 mixture of methylene chloride and methanol as the eluent. There were obtained 95 mg (85% of theory) of (3RS,4RS)-2-[4-[4-(2-phenethyloxy-ethoxy)-phenyl]-piperidin-3-yloxymethyl]-[1,4]naphthoquinone as a red solid; MS: 512 (M+H)⁺.

The BOC derivatives used as starting materials were prepared as follows:

(a) In an analogous manner to that described in Example 1 (g), by alkylating tert-butyl (3RS,4RS)-4-[4-(3-benzyloxy-propoxy)-phenyl]-3-hydroxy-piperidine-1-carboxylate [Example 86 (n)] with 2-chloromethyl-1,4-dimethoxy-naphthalene [J. Org. Chem. (1983), 48(19), 3265-3268] there was obtained (3RS,4RS)-4-[4-(3-benzyloxy-propoxy)-phenyl]-3-(1,4-dimethoxy-naphthalen-2-ylmethoxy)-piperidine-1-carboxylate as a colorless solid; MS: 642 (M+H)⁺.

(b) In an analogous manner to that described in Example 1 (g), by alkylating tert-butyl (RS)-2-(3-bromo-propoxy)-tetrahydro-pyran with 2-hydroxymethyl-thiophene in DMF there was obtained (RS)-2-[3-(thiophen-2-ylmethoxy)-propoxy]-tetrahydro-pyran, which after cleavage of the THP group analogously to Example 53 (c), yielded 3-(thiophen-2-ylmethoxy)-propan-1-ol. Subsequent conversion into the mesylate according to a procedure known from the literature and alkylation effected with the latter analogously to Example 44 (e) of tert-butyl (3RS,4RS)-3-hydroxy-4-(4-hydroxy-phenyl)-piperidine-1-carboxylate gave tert-butyl (3RS,4RS)-3-hydroxy-4-[4-[3-(thiophen-2-ylmethoxy)-propoxy-phenyl]-piperidine-1-carboxylate. By alkylation with 2-chloromethyl-1,4-dimethoxy-naphthalene analogously to Example 1 (g) there was obtained (3RS,4RS)-3-(1,4-dimethoxy-naphthalen-2-ylmethoxy)-4-{4-[3-(thiophen-2-ylmethoxy)-propoxy]-phenyl}-piperidine-1-carboxylate as a pale yellow resin; MS: 648 (M+H)⁺.

(c) In an analogous manner to that described in Example 1 (g), by alkylating tert-butyl (3RS,4RS)-3-hydroxy-4-[4-[3-(thiophen-2-ylmethoxy)-propoxy]-phenyl]-piperidine-1-carboxylate with 4-methylthio-benzyl chloride [J. Org. Chem. (1988), 53(3), 561-569] there was obtained tert-butyl (3RS,4RS)-3-(4-methylsulfanyl-benzyloxy)-4-[4-[3-(thiophen-2-ylmethoxy)-propoxy]-phenyl]-piperidine-1-carboxylate as a yellowish oil; MS: 584 (M+H)⁺.

(d) In an analogous manner to that described in Example 44 (e), by alkylating tert-butyl (3RS,4RS)-3-hydroxy-4-(4-hydroxy-phenyl)-piperidine-1-carboxylate [Example 46 (b)] with (3-bromo-propylsulfanylmethyl)-benzene [J. Org. Chem. (1986), 51, 846-850] in the presence of potassium carbonate there was obtained tert-butyl (3RS,4RS)-4-[4-(3-benzylsulfanyl-propoxy)-phenyl]-3-hydroxy-piperidine-1-carboxylate, alkylation of which with 2-chloromethyl-1,4-dimethoxy-naphthalene analogously to Example 1 (g) gave tert-butyl (3RS,4RS)-4-[4-(3-benzylsulfanyl-propoxy)-phenyl]-3-(1,4-dimethoxy-naphthalen-2-ylmethoxy)-piperidine-1-carboxylate as a pale yellow oil; MS: 584 (M+H)⁺.

(e) In an analogous manner to that described in Example 1 (g), by alkylating tert-butyl (3RS,4RS)-4-[4-(3-benzylsulfanyl-propoxy)-phenyl]-3-hydroxy-piperidine-1-carboxylate with 2-bromomethyl-naphthalene there was obtained tert-butyl (3RS,4RS)-4-[4-(3-benzylsulfanyl-propoxy)-phenyl]-3-(naphthalen-2-ylmethoxy)-piperidine-1-carboxylate as a colorless resin; MS: 598 (M+H)⁺.

(f) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 2-phenethyloxy-ethanol mesylate [J. Med. Chem. (1983), 26 (11), 1570-1576], prepared according to a procedure known from the literature, in the presence of potassium carbonate there was obtained tert-butyl (3RS,4RS)-3-hydroxy-4-[4-(2-phenethyloxy-ethoxy)-phenyl]-piperidine-1-carboxylate, alkylation of which analogously to Example 1 (g) yielded tert-butyl (3RS,4RS)-3-(naphthalen-2-ylmethoxy)-4-[4-(2-phenethyloxy-ethoxy)-phenyl]-piperidine-1-carboxylate as a colorless oil; MS: 582 (M+H)⁺.

(g) In an analogous manner to that described in Example 1 (g), by alkylating tert-butyl (3RS,4RS)-3-hydroxy-4-[4-(2-phenethyloxy-ethoxy)-phenyl]-piperidine-1-carboxylate with 4-methylthio-benzyl chloride [J. Org. Chem. (1988), 53(3), 561-569] there was obtained tert-butyl (3RS,4RS)-3-(4-methylsulfanyl-benzyloxy)-4-[4-(2-phenethyloxy-ethoxy)-phenyl]-piperidine-1-carboxylate as a colorless oil; MS: 578 (M+H)⁺.

(h) In an analogous manner to that described in Example 1 (g), by alkylating tert-butyl (3RS,4RS)-3-hydroxy-4-[4-(2-phenethyloxy-ethoxy)-phenyl]-piperidine-1-carboxylate with 2-chloromethyl-1,4-dimethoxy-naphthalene there was obtained tert-butyl (3RS,4RS)-3-(1,4-dimethoxy-naphthalen-2-ylmethoxy)-4-[4-(2-phenethyloxy-ethoxy)-phenyl]-piperidine-1-carboxylate as a yellow solid; MS: 642 (M+H)⁺.

(i) In an analogous manner to that described in Example 1 (9), by alkylating tert-butyl (3RS,4RS)-4-[4-(2-hydroxy-ethoxy)-phenyl]-3-(naphthalen-2-ylmethoxy)-piperidine-1-carboxylate [Example 53 (c)] with 2-chloro-pyrimidine there was obtained tert-butyl (3RS,4RS)-3-(naphthalen-2-ylmethoxy)-4-[4-[2-(pyrimidin-2-yloxy)-ethoxy]-phenyl]-piperidine-1-carboxylate as a colorless solid; MS: 556 (M+H)⁺.

(j) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 3-phenoxy-propanol mesylate, prepared according to a procedure known from the literature, there was obtained tert-butyl (3RS,4RS)-3-hydroxy-4-[4-(3-phenoxy-propoxy)-phenyl]-piperidine-1-carboxylate, alkylation of which with 2-bromomethyl-naphthalene analogously to Example 1 (g) yielded tert-butyl (3RS,4RS)-3-(naphthalen-2-ylmethoxy)-4-[4-(3-phenoxy-propoxy)-phenyl]-piperidine-1-carboxylate as a colorless solid; MS: 568 (M+H)⁺.

(k) In an analogous manner to that described in Example 44 (e), by alkylating tert-butyl (3RS,4RS)-3-hydroxy-4-(4-hydroxy-phenyl)-piperidine-1-carboxylate [Example 46 (b)] with (E)-(4-bromo-but-2-enyloxy)-benzene there was obtained tert-butyl (E)-(3RS,4RS)-3-hydroxy-4-[4-(4-phenoxy-but-2-enyloxy)-phenyl)-piperidine-1-carboxylate. Subsequent hydrogenation with palladium/charcoal analogously to Example 73 (c) yielded tert-butyl (3RS,4RS)-3-hydroxy-4-[4-(4-phenoxy-butoxy)-phenyl]-piperidine-1-carboxylate, alkylation of which with 2-bromomethyl-naphthalene analogously to Example 1 (g) gave tert-butyl (3RS,4RS)-3-(naphthalen-2-ylmethoxy)-4-[4-(4-phenoxy-butoxy)-phenyl]-piperidine-1-carboxylate as a colorless solid; MS: 582 (M+H)⁺.

The (E)-(4-bromo-but-2-enyloxy)-benzene used as the alkylating agent was obtained by alkylating phenol with 1,4-dibromo-2-butene in an analogous manner to that described in Example 44 (e).

(l) In an analogous manner to that described in Example 1 (g), by alkylating tert-butyl (3RS,4RS)-3-hydroxy-4-[4-(4-phenoxy-butoxy)-phenyl]-piperidine-1-carboxylate with 2-chloromethyl-1,4-dimethoxy-naphthalene there was obtained tert-butyl (3RS,4RS)-3-(1,4-dimethoxy-naphthalen-2-ylmethoxy)-4-[4-(4-phenoxy-butoxy)-phenyl]-piperidine-1-carboxylate as a pale yellow solid; MS: 642 (M+H)⁺.

(m) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 3-phenylthio-propanol mesylate, prepared according to a procedure known from the literature, there was obtained tert-butyl (3RS,4RS)-3-hydroxy-4-[4-(3-phenylsulfanyl-propoxy)-phenyl]-piperidine-1-carboxylate, alkylation of which analogously to Example 1 (g) with 2-chloromethyl-1,4-dimethoxy-naphthalene yielded tert-butyl (3RS,4RS)-3-(1,4-dimethoxy-naphthalen-2-ylmethoxy)-4-[4-(3-phenylsulfanyl-propoxy)-phenyl)-piperidine-1-carboxylate as a yellow, viscous oil; MS: 644 (M+H)⁺.

(n) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 4-phenoxy-but-2-ynyl methanesulfonate there was obtained tert-butyl (3RS,4RS)-3-hydroxy-4-[4-(4-phenoxy-but-2-ynyloxy)-phenyl]-piperidine-1-carboxylate, alkylation of which with 2-bromomethyl-naphthalene gave tert-butyl (3RS,4RS)-3-(naphthalen-2-ylmethoxy)-4-[4-(4-phenoxy-but-2-ynyloxy)-phenyl]-piperidine-1-carboxylate as a pale yellow, viscous oil; MS: 578 (M+H)⁺.

The 4-phenoxy-but-2-ynyl methanesulfonate used as the alkylating agent was obtained in an analogous manner to that described in Example 44 (e) by alkylating phenol with 2-butyne-1,4-diol dimesylate, prepared according to a procedure known from the literature.

(o) In an analogous manner to that described in Example 1 (g), by alkylating tert-butyl (3RS,4RS)-3-hydroxy-4-[4-(4-phenoxy-but-2-ynyloxy)-phenyl]-piperidine-1-carboxylate with 2-chloromethyl-1,4-dimethoxy-naphthalene there was obtained tert-butyl (3RS,4RS)-3-(1,4-dimethoxy-naphthalen-2-ylmethoxy)-4-[4-(4-phenoxy-but-2-ynyloxy)-phenyl]-piperidine-1-carboxylate as a pale yellow solid; MS: 638 (M+H)⁺.

(p) In an analogous manner to that described in Example 1 (g), by alkylating tert-butyl (E)-(3RS,4RS)-3-hydroxy-4-[4-(4-phenoxy-but-2-enyloxy)-phenyl]-piperidine-1-carboxylate [Example 123 (k)] with 2-bromomethyl-naphthalene there was obtained tert-butyl (E)-(3RS,4RS)-3-(naphthalen-2-ylmethoxy)-4-[4-(4-phenoxy-but-2-enyloxy)-phenyl]-piperidine-1-carboxylate as a colorless solid; MS: 580 (M+H)⁺.

(q) In an analogous manner to that described in Example 44 (e), by alkylating tert-butyl (3RS,4RS)-3-hydroxy-4-(4-hydroxy-phenyl)-piperidine-1-carboxylate [Example 46 (b)] with (Z)-4-phenoxy-but-2-enyl methanesulfonate there was obtained tert-butyl (Z)-(3RS,4RS)-3-hydroxy-4-[4-(4-phenoxy-but-2-enyloxy)-phenyl]-piperidine-1-carboxylate, alkylation of which with 2-bromomethyl-naphthalene gave tert-butyl (Z)-(3RS,4RS)-3-(naphthalen-2-ylmethoxy)-4-[4-(4-phenoxy-but-2-enyloxy)-phenyl]-piperidine-1-carboxylate as a colorless solid; MS: 580 (M+H)⁺.

The (Z)-4-phenoxy-but-2-enyl methanesulfonate used as the alkylating agent was obtained in an analogous manner to that described in Example 44 (e) by alkylating phenol with (Z)-4-methylsulfonyloxy-but-2-enyl methanesulfonate, prepared from (Z)-2-butene-1,4-diol according to a procedure known from the literature.

(r) In an analogous manner to that described in Example 1 g), by alkylating tert-butyl (3RS,4RS)-3-hydroxy-4-[4-[3-(thiophen-2-ylmethoxy)-propoxy]-phenyl]-piperidine-1-carboxylate [Example 123 b)] with 2-chloromethyl-4,8-dimethoxy-naphthalene there was obtained tert-butyl (3RS,4RS)-3-(4,8-dimethoxy-naphthalen-2-ylmethoxy)-4-{4-[3-(thiophen-2-ylmethoxy)-propoxy]-phenyl}-piperidine-1-carboxylate as a beige solid; MS: 648 (M+H)⁺.

The 2-chloromethyl-4,8-dimethoxy-naphthalene used as the alkylating agent was prepared as follows:

(α) In an analogous manner to that described in Example 5 b), by reducing methyl 4,8-dimethoxy-naphthalene-2-carboxylate (J. Chem. Soc. 1959, 1024) by means of lithium aluminium hydride there was obtained (4,8-dimethoxy-naphthalen-2-yl)-methanol, MS: 218 (M)⁺, as a colorless solid.

(β) A solution of 3.92 g of methanesulfonyl chloride in 20 ml of methylene chloride was added dropwise to a solution, cooled to −10° C., of 7.7 g (35.3 mmol) of (4,8-dimethoxy-naphthalen-2-yl)-methanol and 4.4 g (38.8 mmol) of triethylamine in 50 ml of methylene chloride. The reaction mixture was stirred at room temperature for 18 hours. For the working-up, the mixture was extracted with 50 ml of ice-cold sodium hydrogen carbonate solution and the aqueous phase was separated and back-extracted with 25 ml of methylene chloride. The combined organic phases were dried over sodium sulfate and subsequently evaporated under reduced pressure. For purification, the residue was filtered over a layer of silica gel using methylene chloride as the eluent. There were obtained 7.2 g of 2-chloromethyl-4,8-dimethoxy-naphthalene as a beige solid; MS: 296 (M)⁺.

(s) In an analogous manner to that described in Example 1 g), by alkylating tert-butyl (3RS,4RS)-3-hydroxy-4-[4-[3-(thiophen-2-ylmethoxy)-propoxy]-phenyl]-piperidine-1-carboxylate [Example 123 b)] with 2-chloromethyl-7-(2-trimethyl-silylethoxymethoxy)-naphthalene [Example 6 u)] there was obtained tert-butyl (3RS,4RS)-4-{4-[3-(thiophen-2-ylmethoxy)-propoxy]-phenyl}-3-[7-(2-trimethylsilanyl-ethoxymethoxy)-naphthalen-2-ylmethoxy]-piperidine-1-carboxylate as a colorless, viscous oil, MS: 734 (M+H)⁺, from which by cleavage of the SEM group by means of hydrogen chloride in methanol there was obtained tert-butyl (3RS,4RS)-3-(7-hydroxy-naphthalen-2-ylmethoxy)-4-{4-[3-(thiophen-2-ylmethoxy)-propoxy]-phenyl}-piperidine-1-carboxylate as a colorless solid; MS: 604 (M+H)⁺.

(t) In an analogous manner to that described in Example 1 g), by alkylating tert-butyl (3RS,4RS)-3-hydroxy-4-[4-[3-(thiophen-2-ylmethoxy)-propoxy]-phenyl]-piperidine-1-carboxylate [Example 123 b)] with 3-chloromethyl-5-methoxy-1-(3-methoxy-propoxy)-naphthalene there was obtained tert-butyl (3RS,4RS)-3-[8-methoxy-4-(3-methoxy-propoxy)-naphthalen-2-ylmethoxy]-4-{4-[3-(thiophen-2-ylmethoxy)-propoxy]-phenyl}-piperidine-1-carboxylate as a pale yellow, viscous oil; MS: 706 (M+H)⁺.

The 3-chloromethyl-5-methoxy-1-(3-methoxy-propoxy)-naphthalene used as the alkylating agent was prepared as follows:

In an analogous manner to that described in Example 44 e), by alkylating methyl 4-hydroxy-8-methoxy-naphthalene-2-carboxylate [Justus Liebigs Ann. Chem. (1967), 702, 94-100] with 3-methoxy-butan-1-ol mesylate, prepared according to a procedure known from the literature, there was obtained ethyl 8-methoxy-4-(3-methoxy-propoxy)-naphthalene-2-carboxylate, reduction of which with lithium aluminium hydride analogously to Example 5 (b) gave [8-methoxy-4-(3-methoxy-propoxy)-naphthalen-2-yl]-methanol. Subsequent chlorination analogously to Example 123 (r) (b) yielded 3-chloromethyl-5-methoxy-1-(3-methoxy-propoxy)-naphthalene as a pale yellow liquid; MS: 276 (M)⁺.

(u) In an analogous manner to that described in Example 1 (g), by alkylating tert-butyl (3RS,4RS)-3-hydroxy-4-[4-[3-(thiophen-2-ylmethoxy)-propoxy]-phenyl]-piperidine-1-carboxylate [Example 123 b)] with (RS)-2-chloromethyl-7-(2,3-dimethoxy-propoxy)-naphthalene there was obtained a mixture of tert-butyl (3RS,4RS)- and (3SR,4SR)-3-[7-[(RS)-2,3-dimethoxy-propoxy]-naphthalen-2-ylmethoxy]-4-{4-[3-(thiophen-2-ylmethoxy)-propoxy]-phenyl}-piperidine-1-carboxylate as a colorless, viscous oil; MS: 706 (M+H)⁺.

The (RS)-2-chloromethyl-7-(2,3-dimethoxy-propoxy)-naphthalene used as the alkylating agent was prepared as follows:

In an analogous manner to that described in Example 44 (e), by alkylating ethyl 7-hydroxy-naphthalene-2-carboxylate (EPA 61800) with (RS)-2,3-dimethoxy-propan-1-ol mesylate (J. Chem. Soc. C, 1966, 415-419), prepared in a manner known from the literature, there was obtained methyl (RS)-7-(2,3-dimethoxy-propoxy)-naphthalene-2-carboxylate, reduction of which with lithium aluminium hydride analogously to Example 5 (b) gave (RS)-[7-(2,3-dimethoxy-propoxy)-naphthalen-2-yl]-methanol. Subsequent chlorination analogously to Example 123 (r) (b) yielded (RS)-2-chloromethyl-7-(2,3-dimethoxy-propoxy)-naphthalene as a colorless solid; MS: 294 (M)⁺.

(v) In an analogous manner to that described in Example 1 (g), by alkylating tert-butyl (3RS,4RS)-3-hydroxy-4-[4-[3-(thiophen-2-ylmethoxy)-propoxy]-phenyl]-piperidine-1-carboxylate [Example 123 (b)] with (RS)-2-chloromethyl-1-(2,3-dimethoxy-propoxy)-naphthalene there was obtained a mixture of tert-butyl (3RS,4RS)- and (3SR,4SR)-3-[1-[(RS)-2,3-dimethoxy-propoxy]-naphthalen-2-ylmethoxy]-4-{4-[3-(thiophen-2-ylmethoxy)-propoxy]-phenyl}-piperidine-1-carboxylate as a colorless oil; MS: 706 (M+H)⁺.

The (RS)-2-chloromethyl-1-(2,3-dimethoxy-propoxy)-naphthalene used as the alkylating agent was prepared as follows:

In an analogous manner to that described in Example 44 (e), with (RS)-2,3-dimethoxy-propan-1-ol mesylate (J. Chem. Soc. C, 1966, 415-419), prepared according to a method known from the literature, there was obtained methyl (RS)-1-(2,3-dimethoxy-propoxy)-naphthalene-2-carboxylate, reduction of which with lithium aluminium hydride analogously to Example 5 (b) gave (RS)-[1-(2,3-dimethoxy-propoxy)-naphthalen-2-yl]-methanol. Subsequent chlorination analogously to Example 123 (r) (b) yielded (RS)-2-chloromethyl-1-(2,3-dimethoxy-propoxy)-naphthalene as a colorless, viscous oil; MS: 294 (M)⁺.

(w) In an analogous manner to that described in Example 1 (g), by alkylating tert-butyl (RS)-2-(3-bromo-propoxy)-tetrahydro-pyran with 3-hydroxymethyl-thiophene in DMF there was obtained (RS)-2-[3-(thiophen-3-ylmethoxy)-propoxy]-tetrahydro-pyran, which after cleavage of the THP group analogously to Example 53 (c) yielded 3-(thiophen-3-ylmethoxy)-propan-1-ol. Subsequent conversion into the mesylate according to a procedure known from the literature and alkylation therewith effected analogously to Example 44 (e) of tert-butyl (3RS,4RS)-3-hydroxy-4-(4-hydroxy-phenyl)-piperidine-1-carboxylate [Example 46 (b)] gave tert-butyl (3RS,4RS)-3-hydroxy-4-[4-[3-(thiophen-3-ylmethoxy)-propoxy]-phenyl]-piperidine-1-carboxylate. By alkylation with 2-chloromethyl-1,4-dimethoxy-naphthalene analogously to Example 1 (g) there was obtained tert-butyl (3RS,4RS)-3-(1,4-dimethoxy-naphthalen-2-ylmethoxy)-4-{4-[3-(thiophen-3-ylmethoxy)-propoxy]-phenyl}-piperidine-1-carboxylate as a pale yellow solid; MS: 648 (M+H)⁺.

(x) In an analogous manner to that described in Example 1 (g), by alkylating tert-butyl (3RS,4RS)-3-hydroxy-4-[4-[3-(thiophen-3-ylmethoxy)-propoxy]-phenyl]-piperidine-1-carboxylate with 2-allyloxy-7-chloromethyl-naphthalene there was obtained tert-butyl (3RS,4RS)-3-(7-allyloxy-naphthalen-2-ylmethoxy)-4-{4-[3-(thiophen-3-ylmethoxy)-propoxy]-phenyl}-piperidine-1-carboxylate, from which after cleavage of the allyl group by means of bis-(triphenylphosphine)-palladium(II) diacetate analogously to Example 152 (e) there was obtained tert-butyl (3RS,4RS)-3-(7-hydroxy-naphthalen-2-ylmethoxy)-4-{4-[3-(thiophen-3-ylmethoxy)-propoxy]-phenyl}-piperidine-1-carboxylate as a yellowish, viscous oil; MS: 604 (M+H)⁺.

The 2-allyloxy-7-chloromethyl-naphthalene used as the alkylating agent was prepared as follows:

In an analogous manner to that described in Example 44 (e), by alkylating ethyl tert-butyl 7-hydroxy-naphthalene-2-carboxylate (EPA 61800) with allyl bromide there was obtained methyl 7-allyloxy-naphthalene-2-carboxylate, reduction of which with lithium aluminium hydride analogously to Example 5 (b) gave (7-allyloxy-naphthalen-2-yl)-methanol. Subsequent chlorination analogously to Example 123 (r) (b) yielded 2-allyloxy-7-chloro-methyl-naphthalene as a colorless solid; MS: 232 (M)⁺.

(y) In an analogous manner to that described in Example 1 (g), by alkylating tert-butyl (3RS,4RS)-3-hydroxy-4-[4-[3-(thiophen-3-ylmethoxy)-propoxy]-phenyl]-piperidine-1-carboxylate with 3-chloromethyl-5-methoxy-1-(3-methoxy-propoxy)-naphthalene [Example 123 (t)] there was obtained tert-butyl (3RS,4RS)-3-[8-methoxy-4-(3-methoxy-propoxy)-naphthalen-2-ylmethoxy]-4{4-[3-(thiophen-3-ylmethoxy)-propoxy]-phenyl}-piperidine-1-carboxylate as a pale yellow, viscous oil; MS: 723 (M+NH₄)⁺.

(z) In an analogous manner to that described in Example 1 (g), by alkylating tert-butyl (3RS,4RS)-3-hydroxy-4-[4-(3-phenoxy-propoxy)-phenyl]-piperidine-1-carboxylate [Example is 123 (j)] with 2-chloromethyl-1,4-dimethoxy-naphthalene there was obtained tert-butyl (3RS,4RS)-3-(1,4-dimethoxy-naphthalen-2-ylmethoxy)-4-[4-(3-phenoxy-propoxy)-phenyl]-piperidine-1-carboxylate as a viscous, yellow oil; MS: 628 (M+H)⁺.

(aa) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 1-(3-bromo-propoxy)-2-methoxy-benzene there was obtained tert-butyl (3RS,4RS)-3-hydroxy-4-{4-[3-(2-methoxy-phenoxy)-propoxy]-phenyl}-piperidine-1-carboxylate, further alkylation of which with 2-chloromethyl-1,4-dimethoxy-naphthalene gave tert-butyl (3RS,4RS)-3-(1,4-dimethoxy-naphthalen-2-ylmethoxy)-4-{4-[3-(2-methoxy-phenoxy)-propoxy]-phenyl}-piperidine-1-carboxylate as a pale yellow solid; MS: 657 (M)⁺.

The 1-(3-bromo-propoxy)-2-methoxy-benzene used as the alkylating agent was obtained as a colorless liquid, MS: 244, 246 (M)⁺, by alkylating 2-methoxyphenol with 1,3-dibromo-propane analogously to Example 44 (e).

(bb) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 1-(3-bromo-propoxy)-3-methoxy-benzene there was obtained tert-butyl (3RS,4RS)-3-hydroxy-4-{4-[3-(3-methoxy-phenoxy)-propoxy)-phenyl}-piperidine-1-carboxylate, further alkylation of which with 2-chloromethyl-1,4-dimethoxy-naphthalene gave tert-butyl (3RS,4RS)-3-(1,4-dimethoxy-naphthalen-2-ylmethoxy)-4-{4-[3-(3-methoxy-phenoxy)-propoxy]-phenyl}-piperidine-1-carboxylate as a pale yellow solid; MS: 658 (M+H)⁺.

The 1-(3-bromo-propoxy)-3-methoxy-benzene used as the alkylating agent was obtained as a colorless liquid, MS: 244, 246 (M)⁺, by alkylating 3-methoxyphenol with 1,3-dibromo-propane analogously to Example 44 (e).

(cc) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 1-(3-bromo-propoxy)-2-chloro-benzene there was obtained tert-butyl (3RS,4RS)-4-{4-[3-(2-chloro-phenoxy)-propoxy]-phenyl}-3-hydroxy-piperidine-1-carboxylate, further alkylation of which with 2-chloromethyl-1,4-dimethoxy-naphthalene gave (3RS,4RS)-4-{4-[3-(2-chloro-phenoxy)-propoxy]-phenyl}-3-(1,4-dimethoxy-naphthalen-2-ylmethoxy)-piperidine-1-carboxylate as a colorless, viscous oil; MS: 662 (M+H)⁺.

The 1-(3-bromo-propoxy)-2-chloro-benzene used as the alkylating agent was obtained as a colorless liquid, MS: 248 (M)⁺, by alkylating 2-chlorophenol with 1,3-dibromo-propane analogously to Example 44 (e).

(dd) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 3-(2-methoxy-phenylsulfanyl)-propyl methanesulfonate there was obtained tert-butyl (3RS,4RS)-3-hydroxy-4-{4-[3-(2-methoxy-phenylsulfanyl)-propoxy]-phenyl}-piperidine-1-carboxylate, further alkylation of which with 2-chloromethyl-1,4-dimethoxy-naphthalene gave tert-butyl (3RS,4RS)-3-(1,4-dimethoxy-naphthalen-2-ylmethoxy)-4-{4-[3-(2-methoxy-phenylsulfanyl)-propoxy]-phenyl}-piperidine-1-carboxylate as a pale yellow, viscous oil; MS: 674 (M+H)⁺.

The 3-(2-methoxy-phenylsulfanyl)-propyl methanesulfonate used as the alkylating agent was prepared as follows:

(α) In an analogous manner to that described in Example 44 e), by alkylating tert-butyl 2-methoxy-thiophenol with (RS)-2-(3-bromo-propoxy)-tetrahydro-pyran there was obtained (RS)-2-[3-(2-methoxy-phenylsulfanyl)-propoxy]-tetrahydro-pyran.

(β) A solution of 9.5 g (33.6 mmol) of (RS)-2-[3-(2-methoxy-phenylsulfanyl)-propoxy]-tetrahydro-pyran and 1.0 g (4 mmol) of pyridinium toluene-4-sulfonate in 100 ml of methanol was heated to reflux for 2 hours. For the working-up, the solvent was distilled off under reduced pressure and then the residue was partitioned between ethyl acetate and saturated sodium hydrogen carbonate solution. The organic phase was subsequently dried over sodium sulfate and evaporated under reduced pressure. The 3-(2-methoxy-phenylsulfanyl)-propane-1-ol was obtained as a yellowish liquid in quantitative yield; MS: 198 (M)⁺. Reaction with mesyl chloride, effected according to a generally known procedure, yielded 3-(2-methoxy-phenylsulfanyl)-propyl methanesulfonate as a pale yellow liquid; MS: 276 (M)⁺.

(ee) In an analogous manner to that described in Example 1 (g), by alkylating tert-butyl (3RS,4RS)-4-[4-(3-benzylsulfanyl-propoxy)-phenyl]-3-hydroxy-piperidine-1-carboxylate [Example 123 d)] with 3-chloromethyl-1,5-dimethoxy-naphthalene [Example 123 r)] there was obtained tert-butyl (3RS,4RS)-4-[4-(3-benzylsulfanyl-propoxy)-phenyl]-3-(4,8-dimethoxy-naphthalen-2-ylmethoxy)-piperidine-1-carboxylate as a yellow, viscous oil; MS: 658 (M+H)⁺.

(ff) In an analogous manner to that described in Example 1 (g), by alkylating tert-butyl (3RS,4RS)-3-hydroxy-4-[4-[3-(2-methoxy-benzyloxy)-propoxy)-phenyl]-piperidine-1-carboxylate with 2-chloromethyl-1,4-dimethoxy-naphthalene there was obtained tert-butyl (3RS,4RS)-3-(1,4-dimethoxy-naphthalen-2-ylmethoxy)-4-{4-[3-(2-methoxy-benzyloxy)-propoxy]-phenyl}-piperidine-1-carboxylate as a pale yellow syrup; MS: 689 (M+NH₄)⁺.

(gg) In an analogous manner to that described in Example 1 (g), by alkylating tert-butyl (3RS,4RS)-3-(7-hydroxy-naphthalen-2-ylmethoxy)-4-{4-[3-(thiophen-2-ylmethoxy)-propoxy]-phenyl}-piperidine-1-carboxylate [Example 123 (s)] with (R)-oxiranylmethyl toluene-4-sulfonate there was obtained a 1:1 mixture of tert-butyl (3R,4R)- and (3S,4S)-3-[(R)-7-oxiranylmethoxy-naphthalen-2-ylmethoxy]-4-{4-[3-(thiophen-2-ylmethoxy)-propoxy]-phenyl}-piperidine-1-carboxylate, MS: 660 (M+H)⁺, as a yellowish, viscous oil. Subsequent epoxide opening analogously to Example 71 (a) with morpholine yielded a 1:1 mixture of tert-butyl (3R,4R)- and (3S,4S)-3-[7-[(R)-2-hydroxy-3-morpholin-4-yl-propoxy]-naphthalen-2-ylmethoxy]-4-{4-[3-(thiophen-2-ylmethoxy)-propoxy]-phenyl}-piperidine-1-carboxylate as a colorless solid; MS: 747 (M+H)⁺.

### Example 124

The following compounds were obtained by cleavage of the BOC group:

1)—From tert-butyl (3RS,4RS)-4-{4-[3-(2-methoxy-phenyl)-[1,2,4]oxadiazol-5-ylmethoxy]-phenyl}-3-(4-methylsulfanyl-benzyloxy)-piperidine-1-carboxylate by means of zinc bromide in methylene chloride, (3RS,4RS)-4-[4-[3-(2-methoxy-phenyl)-[1,2,4]oxadiazol-5-ylmethoxy]-phenyl]-3-(4-methylsulfanyl-benzyloxy)-piperidine as a colorless oil; MS: 518 (M+H)⁺;

2)—from tert-butyl 3-(4-methanesulfonyl-benzyloxy)-4-{4-[3-(2-methoxy-phenyl)-[1,2,4]oxadiazol-5-ylmethoxy]-phenyl}-piperidine-1-carboxylate by means of zinc bromide in methylene chloride, (3RS,4RS)-4-[4-[3-(2-methoxy-phenyl)-[1,2,4]oxadiazol-5-ylmethoxy]-phenyl]-3-(4-methylsulfonyl-benzyloxy)-piperidine as a beige solid; MS: 550 (M+H)⁺;

3)—from a mixture of tert-butyl (3RS,4RS)-3-[4-[(RS)- and -[(SR)-2,3-dimethoxy-propoxy]-8-methoxy-naphthalen-2-ylmethoxy]-4-{4-[3-(2-methoxy-phenyl)-[1,2,4]oxadiazol-5-ylmethoxy]-phenyl}-piperidine-1-carboxylate by means of zinc bromide in methylene chloride a mixture of (3RS,4RS)-3-[4-[(RS)- and -[(SR)-2,3-dimethoxy-propoxy]-8-methoxy-naphthalen-2-ylmethoxy]-4-[4-[3-(2-methoxy-phenyl)-[1,2,4]oxadiazol-5-ylmethoxy]-phenyl]-piperidine as a beige solid; MS: 670 (M+H)⁺;

4)—from tert-butyl (3RS,4RS)-3-(1,4-dimethoxy-naphthalen-2-ylmethoxy)-4-[4-[3-(2-methoxy-phenyl)-[1,2,4]oxadiazol-5-ylmethoxy]-phenyl]-piperidine-1-carboxylate by means of zinc bromide in methylene chloride, (3RS,4RS)-3-(1,4-dimethoxy-naphthalen-2-ylmethoxy)-4-[4-[3-(2-methoxy-phenyl)-[1,2,4]-oxadiazol-5-ylmethoxy]-phenyl]-piperidine as a beige solid; MS: 582 (M+H)⁺;

5)—from tert-butyl (3RS,4RS)-3-(1,4-dimethoxy-naphthalen-2-ylmethoxy)-4-{4-[3-(3,4,5-trimethoxy-phenyl)-[1,2,4]oxadiazol-5-ylmethoxy]-phenyl}-piperidine-1-carboxylate by means of zinc bromide in methylene chloride, (3RS,4RS)-3-(1,4-dimethoxy-naphthalen-2-ylmethoxy)-4-[4-[3-(3,4,5-trimethoxy-phenyl)-[1,2,4]oxadiazol-5-ylmethoxy]-phenyl]-piperidine as a beige solid; MS: 642 (M+H)⁺;

6)—from tert-butyl (3RS,4RS)-3-(4-methylsulfanyl-benzyloxy)-4-[4-(3-thiophen-2-yl-[1,2,4]oxa diazol-5-ylmethoxy)-phenyl]-piperidine-1-carboxylate by means of zinc bromide in methylene chloride, (3RS,4RS)-3-(4-methylsulfanyl-benzyloxy)-4-[4-(3-thiophen-2-yl-[1,2,4]oxa diazol-5-ylmethoxy)-phenyl]-piperidine as a pale yellow, viscous oil; MS: 494 (M+H)⁺;

7)—from tert-butyl (3RS,4RS)-3-(1,4-dimethoxy-naphthalen-2-ylmethoxy)-4-[4-(3-thiophen-2-yl-[1,2,4]oxadiazol-5-ylmethoxy)-phenyl]-piperidine-1-carboxylate by means of zinc bromide in methylene chloride, (3RS,4RS)-3-(1,4-dimethoxy-naphthalen-2-ylmethoxy)-4-[4-(3-thiophen-2-yl-[1,2,4]oxadiazol-5-ylmethoxy)-phenyl]-piperidine as a beige solid; MS: 558 (M+H)⁺;

8)—from tert-butyl 3-[1-(2-methoxy-ethoxymethoxy)-naphthalen-2-ylmethoxy]-4-[4-(3-thiophen-2-yl-[1,2,4]oxadiazol-5-ylmethoxy)-phenyl]-piperidine-1-carboxylate by means of zinc bromide in methylene chloride with simultaneous cleavage of the MEM group, (3RS,4RS)-2-[4-[4-(3-thiophen-2-yl-[1,2,4]oxadiazol-5-ylmethoxy)-phenyl]-piperidin-3-yloxymethyl]-naphthalen-1-ol as a brown solid; MS: 514 (M+H)⁺;

9)—from tert-butyl (3RS,4RS)-3-[1-(2-methoxy-ethoxy)-naphthalen-2-ylmethoxy]-4-[4-(3-thiophen-2-yl-[1,2,4]oxadiazol-5-ylmethoxy)-phenyl]-piperidine-1-carboxylate by means of zinc bromide in methylene chloride, (3RS,4RS)-3-[1-(2-methoxy-ethoxy)-naphthalen-2-ylmethoxy]-4-[4-(3-thiophen-2-yl-[1,2,4]oxadiazol-5-ylmethoxy)-phenyl]-piperidine hydrobromide as a colorless solid; MS: 572 (M+H)⁺;

10)—from tert-butyl (3RS,4RS)-3-(naphthalen-2-ylmethoxy)-4-[4-(3-thiophen-2-yl-[1,2,4]oxadiazol-5-ylmethoxy)-phenyl]-piperidine-1-carboxylate by means of trifluoroacetic acid in methylene chloride, (3RS,4RS)-3-(naphthalen-2-ylmethoxy)-4-[4-(3-thiophen-2-yl-[1,2,4]oxadiazol-5-ylmethoxy)-phenyl]-piperidine trifluoroacetate as a colorless solid; MS: 498 (M+H)⁺;

11)—from tert-butyl (3RS,4RS)-4-[4-(3-furan-2-yl-[1,2,4]-oxadiazol-5-ylmethoxy)-phenyl]-3-(naphthalen-2-ylmethoxy)-piperidine-1-carboxylate by means of trifluoroacetic acid in methylene chloride, (3RS,4RS)-4-[4-(3-furan-2-yl-[1,2,4]oxadiazol-5-ylmethoxy)-phenyl]-3-(naphthalen-2-ylmethoxy)-piperidine trifluoroacetate as a colorless solid; MS: 482 (M+H)⁺;

12)—from tert-butyl (3RS,4RS)-4-[4-[3-(2-chloro-phenyl)-[1,2,4]oxadiazol-5-ylmethoxy]-phenyl]-3-(naphthalen-2-ylmethoxy)-piperidine-1-carboxylate by means of trifluoroacetic acid in methylene chloride, (3RS,4RS)-4-[4-[3-(2-chloro-phenyl)-[1,2,4]oxadiazol-5-ylmethoxy]-phenyl]-3-(naphthalen-2-ylmethoxy)-piperidine trifluoroacetate as a colorless solid; MS: 526 (M+H)⁺;

13)—from tert-butyl (3RS,4RS)-3-(1,4-dimethoxy-naphthalen-2-ylmethoxy)-4-[4-(2-phenyl-oxazol-5-ylmethoxy)-phenyl]-piperidine-1-carboxylate by means of zinc bromide in methylene chloride, (3RS,4RS)-3-(1,4-dimethoxy-naphthalen-2-ylmethoxy)-4-[4-(2-phenyl-oxazol-5-ylmethoxy)-phenyl]-piperidine as a colorless solid; MS: 551 (M+H)⁺;

14)—from tert-butyl (3RS,4RS)-3-(1,4-dimethoxy-naphthalen-2-ylmethoxy)-4-[4-(2-phenyl-thiazol-5-ylmethoxy)-phenyl]-piperidine-1-carboxylate by means of zinc bromide in methylene chloride, {3RS,4RS}-3-(1,4-dimethoxy-naphthalen-2-ylmethoxy)-4-[4-(2-phenyl-thiazol-5-ylmethoxy)-phenyl]-piperidine as a beige solid; MS: 567 (M+H)⁺;

15)—from tert-butyl (3RS,4RS)-3-[1-(2-methoxy-ethoxy)-naphthalen-2-ylmethoxy]-4-[4-(3-phenyl-isoxazol-5-ylmethoxy)-phenyl]-piperidine-1-carboxylate by means of zinc bromide in methylene chloride, (3RS,4RS)-3-[1-(2-methoxy-ethoxy)-naphthalen-2-ylmethoxy]-4-[4-(3-phenyl-isoxazol-5-ylmethoxy)-phenyl-piperidine as a colorless solid; MS: 565 (M+H)⁺;

16)—from tert-butyl (3RS,4RS)-3-(1,4-dimethoxy-naphthalen-2-ylmethoxy)-4-[4-(3-phenyl-isoxazol-5-ylmethoxy)-phenyl]-piperidine-1-carboxylate by means of zinc bromide in methylene chloride, (3RS,4RS)-3-(1,4-dimethoxy-naphthalen-2-ylmethoxy)-4-[4-(3-phenyl-isoxazol-5-ylmethoxy)-phenyl]-piperidine as a colorless solid; MS: 551 (M+H)⁺;

17)—from tert-butyl (3RS,4RS)-3-(1-methoxy-naphthalen-2-ylmethoxy)-4-[4-(3-phenyl-isoxazol-5-ylmethoxy)-phenyl]-piperidine-1-carboxylate by means of trifluoroacetic acid in methylene chloride, (3RS,4RS)-3-(1-methoxy-naphthalen-2-ylmethoxy)-4-[4-(3-phenyl-isoxazol-5-ylmethoxy)-phenyl]-piperidine trifluoroacetate as a colorless solid; MS: 521 (M+H)⁺.

The BOC derivatives used as starting materials were prepared as follows:

(a) In an analogous manner to that described in Example 1 (g), by alkylating tert-butyl (3RS,4RS)-4-(4-allyloxy-phenyl)-3-hydroxy-piperidine-1-carboxylate [Example 86 (b)] with 4-methylthio-benzyl chloride [J. Org. Chem. (1988), 53(3), 561-569] there was obtained tert-butyl (3RS,4RS)-4-(4-allyloxy-phenyl)-3-(4-methylsulfanyl-benzyloxy)-piperidine-1-carboxylate, from which by cleavage of the allyl group by means of bis-(triphenylphosphine)-palladium(II) diacetate analogously to Example 152 (e) there was obtained tert-butyl (3RS,4RS)-4-(4-hydroxy-phenyl)-3-(4-methylsulfanyl-benzyloxy)-piperidine-1-carboxylate. Subsequent alkylation with 5-bromomethyl-3-(2-methoxy-phenyl)-[1,2,4]oxadiazole analogously to Example 44 (e) gave tert-butyl (3RS,4RS)-4-{4-[3-(2-methoxy-phenyl)-[1,2,4]oxadiazol-5-ylmethoxy]-phenyl}-3-(4-methylsulfanyl-benzyloxy)-piperidine-1-carboxylate as a pale yellow, viscous oil; MS: 618 (M+H)⁺.

(b) In an analogous manner to that described in Example 152 (c), by oxidizing tert-butyl (3RS,4RS)-4-{4-[3-(2-methoxy-phenyl)-[1,2,4]oxadiazol-5-ylmethoxy]-phenyl}-3-(4-methylsulfanyl-benzyloxy)-piperidine-1-carboxylate with m-chloroperbenzoic acid there was obtained tert-butyl (3RS,4RS)-3-(4-methanesulfonyl-benzyloxy)-4-{4-[3-(2-methoxy-phenyl)-[1,2,4]oxadiazol-5-ylmethoxy]-phenyl}-piperidine-1-carboxylate as a beige solid; MS: 650 (M+H)⁺.

(c) In an analogous manner to that described in Example 1 (g), by alkylating tert-butyl (3RS,4RS)-4-(4-allyloxy-phenyl)-3-hydroxy-piperidine-1-carboxylate [Example 86 (b)] with (RS)-3-chloromethyl-1-(2,3-dimethoxy-propoxy)-5-methoxy-naphthalene there was obtained a mixture of (3RS,4RS)-4-(4-allyloxy-phenyl)-3-[4-[(RS)- and [(SR)-2,3-dimethoxy-propoxy]-8-methoxy-naphthalen-2-ylmethoxy]-piperidine-1-carboxylate, from which by cleavage of the allyl group by means of bis-(triphenylphosphine)-palladium(II) diacetate analogously to Example 152 (e) there was obtained a mixture of tert-butyl (3RS,4RS)-3-[4-[(RS)- and [(SR)-2,3-dimethoxy-propoxy]-8-methoxy-naphthalen-2-ylmethoxy]-4-(4-hydroxy-phenyl)-piperidine-1-carboxylate. Subsequent alkylation with 5-bromomethyl-3-(2-methoxy-phenyl)-[1,2,4]oxadiazole analogously to Example 44 (e) gave a mixture of tert-butyl (3RS,4RS)-3-[4-(RS)- and [(SR)-2,3-dimethoxy-propoxy]-8-methoxy-naphthalen-2-ylmethoxy]-4-{4-[3-(2-methoxy-phenyl)-[1,2,4]oxadiazol-5-ylmethoxy]-phenyl}-piperidine-1-carboxylate as a beige solid; MS: 770 (M+H)⁺.

The (RS)-3-chloromethyl-1-(2,3-dimethoxy-propoxy)-5-methoxy-naphthalene used as the alkylating agent was prepared as follows:

Ethyl 4-acetoxy-8-methoxy-naphthalene-2-carboxylate [Chem. Pharm. Bull. 19 (6), 1245-1256 (1971)] was saponified by means of aqueous potassium carbonate in ethanol to ethyl 4-hydroxy-8-methoxy-naphthalene-2-carboxylate, which in analogy to Example 44 (e) was alkylated in the presence of potassium carbonate with (RS)-2,3-dimethoxy-propyl methanesulfonate, obtained from (RS)-2,3-dimethoxy-propan-1-ol [J. Chem. Soc. (1931), 450] according to a procedure known from the literature, to give ethyl (RS)-4-(2,3-dimethoxy-propoxy)-8-methoxy-naphthalen-2-carboxylate. Subsequent reduction by means of lithium aluminium hydride analogously to Example 5 (b) gave (RS)-[4-(2,3-dimethoxy-propoxy)-8-methoxy-naphthalen-2-yl]-methanol, which was converted in analogy to Example 5 (c) into (RS)-3-chloromethyl-1-(2,3-dimethoxy-propoxy)-5-methoxy-naphthalene; MS: 324 (M)⁺.

(d) Alkylation of tert-butyl (3RS,4RS)-3-(1,4-dimethoxy-naphthalen-2-ylmethoxy)-4-(4-hydroxy-phenyl)-piperidine-1-carboxylate [Example 152 (e)] with 5-bromomethyl-3-(2-methoxy-phenyl)-[1,2,4]oxadiazole analogously to Example 44 (e) gave tert-butyl (3RS,4RS)-3-(1,4-dimethoxy-naphthalen-2-ylmethoxy)-4-[4-[3-(2-methoxy-phenyl)-[1,2,4]oxadiazol-5-ylmethoxy]-phenyl]-piperidine-1-carboxylate as a colorless solid; MS: 682 (M+H)⁺.

(e) Alkylation of tert-butyl (3RS,4RS)-3-(1,4-dimethoxy-naphthalen-2-ylmethoxy)-4-(4-hydroxy-phenyl)-piperidine-1-carboxylate [Example 152 (e)] with 5-bromomethyl-3-(3,4,5-trimethoxy-phenyl)-[1,2,4]oxadiazole analogously to Example 44 (e) gave tert-butyl (3RS,4RS)-3-(1,4-dimethoxy-naphthalen-2-ylmethoxy)-4-{4-[3-(3,4,5-trimethoxy-phenyl)-[1,2,4]oxadiazol-5-ylmethoxy]-phenyl}-piperidine-1-carboxylate as a colorless solid; MS: 742 (M+H)⁺.

(f) Alkylation of tert-butyl (3RS,4RS)-4-(4-hydroxy-phenyl)-3-(naphthalen-2-ylmethoxy)-piperidine-1-carboxylate [Example 61 (c)] with 5-bromomethyl-3-(2-chloro-phenyl)-[1,2,4)oxadiazole analogously to Example 44 (e) gave tert-butyl (3RS,4RS)-4-[4-[3-(2-chloro-phenyl)-[1,2,4]oxadiazol-5-ylmethoxy]-phenyl]-3-(naphthalen-2-ylmethoxy)-piperidine-1-carboxylate as a yellow solid; MS: 626 (M+H)⁺.

(g) Alkylation of tert-butyl (3RS,4RS)-4-(4-hydroxy-phenyl)-3-(4-methylsulfanyl-benzyloxy)-piperidine-1-carboxylate with 5-bromomethyl-3-thiophen-2-yl-[1,2,4]oxadiazole analogously to Example 44 (e) gave tert-butyl (3RS,4RS)-3-(4-methylsulfanyl-benzyloxy)-4-[4-(3-thiophen-2-yl-[1,2,4]oxa diazol-5-ylmethoxy)-phenyl]-piperidine-1-carboxylate as a colorless solid; MS: 594 (M+H)⁺.

(h) Alkylation of tert-butyl (3RS,4RS)-3-(1,4-dimethoxy-naphthalen-2-ylmethoxy)-4-(4-hydroxy-phenyl)-piperidine-1-carboxylate [Example 152 (e)] with 5-bromomethyl-3-thiophen-2-yl-[1,2,4]oxadiazole analogously to Example 44 (e) gave tert-butyl (3RS,4RS)-3-(1,4-dimethoxy-naphthalen-2-ylmethoxy)-4-[4-(3-thiophen-2-yl-[1,2,4]oxadiazol-5-ylmethoxy)-phenyl]-piperidine-1-carboxylate as a colorless, viscous oil; MS: 658 (M+H)⁺.

(i) In an analogous manner to that described in Example 1 (g), by alkylating tert-butyl (3RS,4RS)-4-(4-allyloxy-phenyl)-3-hydroxy-piperidine-1-carboxylate [Example 86 (b)] with 2-chloromethyl-1-(2-methoxy-ethoxymethoxy)-naphthalene there was obtained tert-butyl (3RS,4RS)-4-(4-allyloxy-phenyl)-3-[1-(2-methoxy-ethoxymethoxy)-naphthalen-2-ylmethoxy]-piperidine-1-carboxylate, from which by cleavage of the allyl group by means of bis-(triphenylphosphine)-palladium(II) diacetate analogously to Example 152 (e) there was obtained tert-butyl (3RS,4RS)-4-(4-hydroxy-phenyl)-3-[1-(2-methoxy-ethoxymethoxy)-naphthalen-2-ylmethoxy]-piperidine-1-carboxylate. Subsequent alkylation with 5-bromomethyl-3-thiophen-2-yl-[1,2,4]oxadiazole analogously to Example 44 (e) gave tert-butyl (3RS,4RS)-3-[1-(2-methoxy-ethoxymethoxy)-naphthalen-2-ylmethoxy]-4-[4-(3-thiophen-2-yl-[1,2,4]oxadiazol-5-ylmethoxy)-phenyl]-piperidine-1-carboxylate as a colorless, viscous oil; MS: 702 (M+H)⁺.

The 2-chloromethyl-1-(2-methoxy-ethoxymethoxy)-naphthalene used as the alkylating agent was prepared as follows:

(α) A solution of 2.3 g (11.4 mmol) of methyl 1-hydroxy-naphthalene-2-carboxylate in 15 ml of dry tetrahydrofuran was treated with 0.51 g (17 mmol) of sodium hydride (80%) and subsequently 2.13 g of 2-methoxyethoxymethyl chloride were added dropwise while cooling with ice. After 3 hours at room temperature the solution was extracted with aqueous sodium hydrogen carbonate solution and the organic phase was separated, dried over sodium sulfate and evaporated under reduced pressure. For purification, the crude product was chromatographed on silica gel using a 9:1 mixture of methylene chloride and ether as the eluent. In addition to 1.55 g of starting material there were obtained 1.2 g of methyl 1-(2-methoxy-ethoxymethyl)-naphthalene-2-carboxylate; MS: 290 (M)⁺.

(β) In an analogous manner to that described in Example 5 (b)-(c), from methyl 1-(2-methoxy-ethoxymethoxy)-naphthalene-2-carboxylate by reduction by means of lithium aluminium hydride there was obtained [1-(2-methoxy-ethoxymethoxy)-naphthalen-2-yl]-methanol, which was then converted into 2-chloromethyl-1-(2-methoxy-ethoxymethoxy)-naphthalene; MS: 280 (M)⁺.

(j) Alkylation of tert-butyl (3RS,4RS)-4-(4-hydroxy-phenyl)-3-[1-(2-methoxy-ethoxy)-naphthalen-2-ylmethoxy]-piperidine-1-carboxylate [Example 122 (h)] with 5-bromomethyl-3-thiophen-2-yl-[1,2,4]oxadiazole analogously to Example 44 (e) gave tert-butyl (3RS,4RS)-3-[1-(2-methoxy-ethoxy)-naphthalen-2-ylmethoxy]-4-[4-(3-thiophen-2-yl-[1,2,4]oxadiazol-5-ylmethoxy)-phenyl]-piperidine-1-carboxylate as a colorless, viscous oil; MS: 672 (M+H)⁺.

(k) Alkylation of tert-butyl (3RS,4RS)-4-(4-hydroxy-phenyl)-3-(naphthalen-2-ylmethoxy)-piperidine-1-carboxylate [Example 61 (c)] with 5-bromomethyl-3-thiophen-2-yl-[1,2,4]oxadiazole analogously to Example 44 (e), gave tert-butyl (3RS,4RS)-3-(naphthalen-2-ylmethoxy)-4-[4-(3-thiophen-2-yl-[1,2,4]oxadiazol-5-ylmethoxy)-phenyl]-piperidine-1-carboxylate as a colorless, viscous oil; MS: 597 (M+H)⁺.

(l) Alkylation of tert-butyl (3RS,4RS)-4-(4-hydroxy-phenyl)-3-(naphthalen-2-ylmethoxy)-piperidine-1-carboxylate [Example 61 (c)] with 5-bromomethyl-3-furan-2-yl-[1,2,4]oxadiazole analogously to Example 44 (e) gave tert-butyl (3RS,4RS)-4-[4-(3-furan-2-yl-[1,2,4]oxadiazol-5-ylmethoxy)-phenyl]-3-(naphthalen-2-ylmethoxy)-piperidine-1-carboxylate as a colorless solid; MS: 582 (M+H)⁺.

(m) Alkylation of tert-butyl (3RS,4RS)-3-(1,4-dimethoxy-naphthalen-2-ylmethoxy)-4-(4-hydroxy-phenyl)-piperidine-1-carboxylate [Example 152 (e)] with 4-chloromethyl-2-phenyl-oxazole [Arch. Pharmazie (1971), 425] analogously to Example 44 (e) gave tert-butyl (3RS,4RS)-3-(1,4-dimethoxy-naphthalen-2-ylmethoxy)-4-[4-(2-phenyl-oxazol-5-ylmethoxy)-phenyl]-piperidine-1-carboxylate as a colorless solid; MS: 651 (M+H)⁺.

(n) Alkylation of tert-butyl (3RS,4RS)-3-(1,4-dimethoxy-naphthalen-2-ylmethoxy)-4-(4-hydroxy-phenyl)-piperidine-1-carboxylate [Example 152 (e)] with 4-chloromethyl-2-phenyl-thiazole [Chem. Ber. (1961), 2887] analogously to Example 44 (e) gave tert-butyl (3RS,4RS)-3-(1,4-dimethoxy-naphthalen-2-ylmethoxy)-4-[4-(2-phenyl-thiazol-5-ylmethoxy)-phenyl]-piperidine-1-carboxylate as a pale yellow solid; MS: 667 (M+H)⁺.

(o) In an analogous manner to that described in Example 1 (g), by alkylating tert-butyl (3RS,4RS)-4-(4-allyloxy-phenyl)-3-hydroxy-piperidine-1-carboxylate [Example 86 (b)] with 2-chloromethyl-1-(2-methoxy-ethoxy)-naphthalene [Example 122 (h)] there was obtained tert-butyl (3RS,4RS)-4-(4-allyloxy-phenyl)-3-[1-(2-methoxy-ethoxy)-naphthalen-2-ylmethoxy]-piperidine-1-carboxylate, from which by cleavage of the allyl group by means of bis-(triphenylphosphine)-palladium(II) diacetate analogously to Example 152 (e) there was obtained tert-butyl (3RS,4RS)-4-(4-hydroxy-phenyl)-3-[ ]-(2-methoxy-ethoxy)-naphthalen-2-ylmethoxy]-piperidine-1-carboxylate. Subsequent alkylation with 3-phenyl-isoxazol-5-ylmethyl methanesulfonate analogously to Example 44 (e) gave tert-butyl (3RS,4RS)-3-[1-(2-methoxy-ethoxy)-naphthalen-2-ylmethoxy]-4-[4-(3-phenyl-isoxazol-5-ylmethoxy)-phenyl]-piperidine-1-carboxylate as a colorless solid; MS: 665 (M+H)⁺.

(p) In an analogous manner to that described in Example 44 (e), by alkylating tert-butyl (3RS,4RS)-3-hydroxy-4-(4-hydroxy-phenyl)-piperidine-1-carboxylate [Example 46 (b)] with 3-phenyl-isoxazol-5-ylmethyl methanesulfonate there was obtained tert-butyl (3RS,4RS)-3-hydroxy-4-[4-(3-phenyl-isoxazol-5-ylmethoxy)-phenyl]-piperidine-1-carboxylate, further alkylation of which with 2-chloromethyl-1,4-dimethoxy-naphthalene [J. Org. Chem. (1983), 48(19),3265-3268)] analogously to Example 1 (g) yielded tert-butyl (3RS,4RS)-3-(1,4-dimethoxy-naphthalen-2-ylmethoxy)-4-[4-(3-phenyl-isoxazol-5-ylmethoxy)-phenyl]-piperidine-1-carboxylate as a colorless solid; MS: 651 (M+H)⁺.

(q) Alkylation of tert-butyl (3RS,4RS)-3-hydroxy-4-[4-(3-phenyl-isoxazol-5-ylmethoxy)-phenyl]-piperidine-1-carboxylate with 1-methoxy-2-bromomethyl-naphthalene [Example 7 (f)] analogously to Example 1 (g) yielded tert-butyl (3RS,4RS)-3-(1-methoxy-naphthalen-2-ylmethoxy)-4-[4-(3-phenyl-isoxazol-5-ylmethoxy)-phenyl]-piperidine-1-carboxylate as a colorless solid; MS: 621 (M+H)⁺.

The preparation of the substituted 5-bromomethyl-[1,2,4]oxadiazoles used as alkylating agents:
5-bromomethyl-3-(2-methoxy-phenyl)-[1,2,4]oxadiazole,
5-bromomethyl-3-(3,4,5-trimethoxy-phenyl)-[1,2,4]oxadiazole,
5-bromomethyl-3-(2-chloro-phenyl)-[1,2,4]oxadiazole,
5-bromomethyl-3-thiophen-2-yl-[1,2,4]oxadiazole and
5-bromomethyl-3-furan-2-yl-[1,2,4]oxadiazole was effected analogously to the procedure described in J. Med. Chem. 1986, 26, 2174-2183

The 3-phenyl-isoxazol-5-ylmethyl methanesulfonate used as the alkylating agent was synthesized as follows:

(α) 1.47 g (11 mmol) of N-chlorosuccinimide were added at −30° C. to a solution of 1.21 g of benzaldoxime in 10 ml of methylene chloride. After 2 hours a solution of 1.0 g of triethylamine and 1.4 g of (RS)-tetrahydro-2-(2-propynyloxy)-2H-pyran in 5 ml of methylene chloride was added dropwise. The reaction mixture was stirred at room temperature for 5 hours, the solvent was thereafter distilled off and, for purification, the crude product was chromatographed on silica gel using methylene chloride as the eluent. There were obtained 1.8 g of (RS)-3-phenyl-5-(tetrahydro-pyran-2-yloxymethyl)-isoxazole as a colorless liquid; MS: 259 (M)⁺.

(β) Subsequent cleavage of the THP group was effected analogously to Example 53 (γ) The thus-obtained (3-phenyl-isoxazole-5-yl)-methanol was converted according to a procedure known in the literature into 3-phenyl-izoxazol-5-ylmethyl methanesulfonate and was thereby obtained as a colorless solid; MS: 253 (M)⁺.

### Example 125

(a) A suspension of 13.32 g (0.1 mmol) of (E)-3-(4-pyridyl)-2-propenal [Tetrahedron Letters 26, 6447 (1985)] and 19.92 g (0.1 mol) of 2-(phenylsulfonyl)-acetamide [Synthesis 1987, 56] in 300 ml of ethanol was treated dropwise at room temperature while stirring during 45 minutes with 20 ml of Triton B solution (40% in methanol) and subsequently stirred at room temperature for 16 hours and under reflux for 90 minutes. After cooling the mixture was treated with 100 ml of glacial acetic acid and subsequently heated under reflux for 2.5 hours. Then, it was concentrated in a water-jet vacuum, treated with 200 ml of water followed by 16.4 g (0.2 mol) of sodium acetate and again concentrated. The residue was taken up in methylene chloride, filtered, the filtrate was concentrated and the thus-obtained residue was recrystallized from isopropanol. There were thus obtained 3.9 g (23% of theory) of 1H-[4,4′]bipyridin-2-one in the the of slightly yellow crystals; m.p. 263-265° C.

(b) 15 ml of methyl iodide were added to a suspension of 9.0 g (52.3 mmol) of 1H-[4,4′]bipyridin-2-one in 150 ml of N,N-dimethylformamide and the reaction mixture was stirred at room temperature for 20 hours. Subsequently, 300 ml of ether were added dropwise and the precipitate which thereby formed was filtered off, washed with ether and dried in a high vacuum. There were thus obtained 15.8 g (96% of theory) of 1-methyl-4-(2-oxo-1,2-dihydro-pyridin-4-yl)-pyridinium iodide in the form of slightly yellow crystals; m.p.: 264-266° C.

(c) 5.3 g (16.9 mmol) of 1-methyl-4-(2-oxo-1,2-dihydro-pyridin-4-yl)-pyridinium iodide were suspended in 100 ml of methanol and treated portionwise under argon at room temperature with 1.1 g (29 mmol) of sodium borohydride. Thereupon, the reaction mixture was heated under reflux for 5 hours and subsequently concentrated in a water-jet vacuum. The thus-obtained residue was partitioned between saturated sodium chloride solution and methylene chloride/methanol (9:1) and the combined methylene chloride phases were dried over magnesium sulfate and concentrated. Recrystallization of the residue from methanol/ethyl acetate finally yielded 2.7 g (84% of theory) of 1′-methyl-1′,2′,3′,6′-tetrahydro-1H-[4,4′]bipyridin-2-one in the form of slightly yellow crystals: m.p.: 250-252° C.

(d) 0.88 g (4.6 mmol) of 1′-methyl-1′,2′,3′,6′-tetrahydro-1H-[4,4′]bipyridin-2-one, 180 mg (2.4 mmol) of lithium carbonate and 2 g of molecular seive (4 .ANG.) were suspended in 20 ml of 1,2-dichloroethane, treated with 1.1 ml (10 mmol) of 1-chloroethyl chloroformate and heated under reflux for 18 hours. Thereupon, the reaction mixture was concentrated in a water-jet vacuum and stirred at room temperature for 18 hours with 2.2 g (10 mmol) of di-tert-butyl dicarbonate and 2 g (24 mmol) of sodium hydrogen carbonate in 60 ml of dioxan/water (2:1). Subsequently, the mixture was again concentrated in a water-jet vacuum and the residue thus obtained was partitioned between methylene chloride and 0.1N hydrochloric acid. The combined methylene chloride phases were dried over magnesium sulfate and recrystallized from ether. There was thus obtained 0.24 g (19% of theory) of tert-butyl 2′-oxo-1′,2′,3,6-tetrahydro-2H-[4,4′]bipyridine-1-carboxylate in the form of a yellowish solid; MS: 277 (M+H)⁺.

(e) 0.50 g (1.8 mmol) of tert-butyl 2′-oxo-1′,2′,3,6-tetrahydro-2H-[4,4′]bipyridine-1-carboxylate, 0.50 g (2.5 mmol) of (2-bromoethoxy)-benzene and 0.35 g (2.5 mmol) of potassium carbonate were heated at 75° C. for 20 hours in 6 ml of acetonitrile. Thereupon, the reaction mixture was concentrated in a water-jet vacuum and partitioned between water and methylene chloride, the combined methylene chloride phases were dried over magnesium sulfate, concentrated and the thus-obtained residue was chromatographed on silica gel with methylene chloride/ethyl acetate (1:1). There was thus obtained 0.41 g (58% of theory) of tert-butyl 2′-oxo-1′-(2-phenoxy-ethyl)-1′,2′,3,6-tetrahydro-2H-[4,4′]bipyridine-1-carboxylate in the form of an amorphous yellowish solid; MS: 397 (M+H)⁺.

(f) 0.20 g (0.5 mmol) of tert-butyl 2′-oxo-1′-(2-phenoxy-ethyl)-1′,2′,3,6-tetrahydro-2H-[4,4′]bipyridine-1-carboxylate was dissolved in 5 ml of 1,2-dimethoxyethane, treated with 1.5 mg of 1 molar borane-tetrahydrofuran solution and stirred at room temperature for 48 hours. Thereupon, a further 1.0 ml of 1 molar borane-tetrahydrofuran solution was added and the mixture was stirred at room temperature for a further 60 hours. Subsequently, 2.5 ml of 50% KOH in water followed by 2.5 ml of 30% hydrogen peroxide solution in water were added while cooling with ice and the reaction mixture was heated under reflux for 2 hour. Now, the reaction solution was partitioned between water and methylene chloride, the combined methylene chloride phases were dried over magnesium sulfate, concentrated and the residue thus obtained was chromatographed on silica gel with methylene chloride/methanol (95:5). There were thus obtained 23 mg (11% of theory) of tert-butyl (3RS,4RS)-3-hydroxy-2′oxo-1′-(2-phenoxy-ethyl)-3,4,5,6,1′,2′-hexahydro-2H-[4,4′]-bipyridine-1-carboxylate in the form of an amorphous, yellowish solid; MS: 414 (M)⁺.

(g) A solution of 21 mg (0.051 mmol) of tert-butyl (3RS,4RS)-3-hydroxy-2′oxo-1′-(2-phenoxy-ethyl)-3,4,5,6,1′,2′-hexahydro-2H-[4,4′]-bipyridine-1-carboxylate and 15 mg (0.068 mmol) of 2-bromomethylnaphthalene in 0.5 ml of dimethylformamide was treated with 4.0 mg (0.083 mmol) of sodium hydride (50% dispersion in refined oil) and the mixture was stirred at room temperature for 0.5 hour. Subsequently, the reaction mixture was partitioned between ether and water and the combined ether phases were dried over magnesium sulfate and concentrated in a water-jet vacuum. The crude product was chromatographed on silica gel with methylene chloride/methanol (95:5). There were thus obtained 20 mg (71% of theory) of tert-butyl (3′RS,4′RS)-3′-(naphthalen-2-ylmethoxy)-1-(2-phenoxy-ethyl)-1′,2′,3′,4′,5′,6′-hexahydro-1H-[4,4′]bipyridin-2-one-1′-carboxylate in the form of an amorphous, yellowish solid; MS: 555 (M+H)⁺.

(h) 20 mg (0.036 mmol) of tert-butyl (3′RS,4′RS)-3′-(naphthalen-2-ylmethoxy)-1-(2-phenoxy-ethyl)-1′,2′,3′,4′,5′,6′-hexahydro-1H-[4,4′]bipyridin-2-one-1′-carboxylate were dissolved in 3 ml of methylene chloride, treated with 40 mg (0.18 mmol) of anhydrous zinc bromide and stirred at room temperature for 3 hours. Thereupon, the reaction mixture was poured into aqueous sodium carbonate solution this was extracted with methylene chloride. The combined methylene chloride phases were dried over magnesium sulfate and concentrated, and the thus obtained residue was chromatographed on silica gel with a 9:1 mixture of methylene chloride and methanol as the eluent. Therefrom there were obtained 5.8 mg (35% of theory) of (3′RS,4′RS)-3′-(naphthalen-2-ylmethoxy)-1-(2-phenoxy-ethyl)-1′,2′,3′,4′,5′,6′-hexahydro-1H-[4,4′]bipyridin-2-one in the form of and amorphous, colorless solid; MS: 455 (M+H)⁺.

### Example 126

(a) 41 g (0.173 mol) of 2,5-dibromopyridine and 20.1 g (0.173 mol) of 3-phenyl-1-propyne were dissolved in 450 ml of triethylamine under argon and with the exclusion of moisture, treated while cooling with ice with 740 mg (3.88 mmol) of copper(I) iodide and 2.7 g (3.88 mmol) of bis-(triphenylphosphine)-palladium dichloride and stirred at 0-5° C. for 1 hour and at room temperature for 1 hour. Now, the reaction solution was poured on to ice-water and extracted with methylene chloride, the combined methylene chloride phases were dried over magnesium sulfate, concentrated and the thus obtained residue was chromatographed on silica gel once with methylene chloride and once with hexane/ethyl acetate (9:1). There were thus obtained 27 g (57% of theory) of 5-bromo-2-(3-phenyl-prop-1-ynyl)-pyridine as a colorless solid; MS: 271, 273 (M)⁺.

(b) 17 g (0.062 mol) of 5-bromo-2-(3-phenyl-prop-1-ynyl)-pyridine were dissolved in 300 ml of ethanol, treated with 150 mg of platinum oxide and hydrogenated in a hydrogen atmosphere for 1 hour. Subsequently, the reaction mixture was filtered over a 0.8μ cellulose filter and concentrated in a water-jet vacuum. The thus obtained residue was chromatographed on silica gel with methylene chloride. There were thus obtained 5.2 g (30% of theory) of 5-bromo-2-(3-phenyl-propyl)-pyridine in the form of a yellowish oil; MS: 171, 173 (M-vinylbenzole)⁺.

(c) 100 ml of a 1.6 molar n-butyllithium solution in hexane (about 0.16 mol) were added dropwise to 21.5 ml (0.152 mol) of diisopropylamine dissolved in 145 ml of tetrahydrofuran under argon and with the exclusion of moisture in such a manner that the temperature did not rise above −70° C. Thereupon, 29 g (0.145 mol) of tert-butyl 4-piperidone-1-carboxylate dissolved in 145 ml of tetrahydrofuran were added dropwise during 45 minutes, with the temperature being held below −70° C. After stirring for 10 minutes at the same temperature a solution of 56 g (0.157 mol) of N-phenyl-bis-(trifluoromethanesulfonamide) in 145 ml of tetrahydrofuran was added dropwise within 30 minutes in such a manner that the temperature did not rise above −70° C. Thereupon, the reaction mixture was left to warm to 0° C. and was stirred at this temperature for a further 3 hours. Subsequently, the reaction solution was concentrated at 40° C. in a water-jet vacuum and the thus obtained residue was chromatographed on basic Alox with hexane/ethyl acetate (9:1). There were thus obtained 41 g (85% of theory) of tert-butyl 4-trifluoromethylsulfonyloxy-3,6-dihydro-2H-pyridine-1-carboxylate in the form of a colorless oil; MS: 332 (M+H)⁺.

(d) 1.05 g (3.8 mmol) of 5-bromo-2-(3-phenyl-propyl)-pyridine, 1.12 ml (5.4 mmol) of hexamethyldistannate, 100 mg (0.086 mmol) of tetrakis-(triphenylphosphine)-palladium, 3 g of molecular seive (4 Å) and a few crystals of 2,6-di-tert-butyl-p-cresol were suspended in 15 ml of dioxan and the reaction mixture was stirred at 100° C. under argon for 3 hours. Thereupon, the reaction mixture was filtered, concentrated in a water-jet vacuum and the residue was chromatographed on silica gel with hexane/ethyl acetate (3:1). There was thus obtained 0.93 g (68% of theory) of 2-(3-phenyl-propyl)-5-trimethylstannyl-pyridine in the form of a yellowish oil; MS: 362 (M+H)⁺.

(e) 0.93 g (2.6 mmol) of 2-(3-phenyl-propyl)-5-trimethylstannyl-pyridine, 0.9 g (2.7 mmol) of tert-butyl 4-trifluoro-methylsulfonyloxy-3,6-dihydro-2H-pyridine-1-carboxylate, 0.345 g (8.0 mmol) of lithium chloride, 100 mg (0.086 mmol) of tetrakis-(triphenylphosphine)-palladium, 3 g of molecular seive (4 Å) and a few crystals of 2,6-di-tert-butyl-p-cresol were suspended in 40 ml of 1,2-dimethoxyethane and the reaction mixture was stirred under reflux for 8 hours under argon. Thereupon, the mixture was filtered, concentrated in a water-jet vacuum and the residue was chromatographed on silica gel with methylene chloride/ether (3:2). There was thus obtained 0.411 g (42% of theory) of tert-butyl 6-(3-phenyl-propyl)-3′,6′-dihydro-2′H-[3,4′]bipyridine-1′-carboxylate in the form of a yellowish oil; MS: 379 (M+H)⁺.

(f) 0.587 g (1.55 mmol) of tert-butyl 6-(3-phenyl-propyl)-3′,6′-dihydro-2′H-[3,4′]bipyridine-1′-carboxylate were dissolved in 8 ml of 1,2-dimethoxyethane, treated with 6 ml of 1 molar borane-tetrahydrofuran solution and stirred at 60° to 65° C. for 4 hours in a flask closed with a Teflon stopper. Thereafter, a further 3 ml of 1 molar borane-tetrahydrofuran solution were added and after 24 hours a further 2.2 ml of 1 molar borane-tetrahydrofuran solution were added and the mixture was stirred at 60° to 65° C. for a total of 48 hours. Subsequently, while cooling with ice, 7.0 ml of 50% KOH solution in water followed by 6.0 ml of 30% hydrogen peroxide solution in water were added and the reaction mixture was heated under reflux for 2 hours. Now, the reaction solution was partitioned between water and methylene chloride, the combined methylene chloride phases were dried over magnesium sulfate, concentrated and the thus obtained residue was chromatographed on silica gel with ether/methanol (99:1). There were thus obtained 211 mg (34% of theory) of tert-butyl (3′RS,4′RS)-3′-hydroxy-6-(3-phenyl-propyl)-3′,4′,5′,6′-tetra-hydro-2′H-[3,4′]bipyridine-1′-carboxylate in the form of an amorphous, colorless solid; MS: 397 (M+H)⁺.

(g) In an analogous manner to that described in Example 125 (g), from tert-butyl (3′RS,4′RS)-3′-hydroxy-6-(3-phenyl-propyl)-3′,4′,5′,6′-tetrahydro-2′H-[3,4′]bipyridine-1′-carboxylate and 2-bromomethylnaphthalene there was obtained tert-butyl (3′RS,4′RS)-3′-(naphthalen-2-ylmethoxy)-6-(3-phenyl-propyl)-3′,4′,5′,6′-tetrahydro-2′H-[3,4′]bipyridine-1′-carboxylate in the form of an amorphous, colorless solid; MS: 537 (M+H)⁺.

(h) In an analogous manner to that described in Example 125 (h), from tert-butyl (3′RS,4′RS)-3′-(naphthalen-2-ylmethoxy)-6-(3-phenyl-propyl)-3′,4′,5′,6′-tetrahydro-2′H-[3,4′]bipyridine-1′-carboxylate by cleavage of the BOC group by means of anhydrous zinc bromide there was obtained (3′RS,4′RS)-3′-(naphthalen-2-ylmethoxy)-6-(3-phenyl-propyl)-1′,2′,3′,4′,5′,6′-hexahydro-[3,4′]bipyridine in the form of an amorphous, colorless foam; MS: 437 (M+H)⁺.

### Example 127

(a) 1.24 g (5.7 mmol) of 5-bromo-2-(3-hydroxy-propyl)-pyridine [J. Org. Chem. 53, 386 (1988)] were dissolved in 4 ml of N,N-dimethylformamide, treated with 0.7 ml (5.9 mmol) of benzyl bromide followed by 285 mg (about 5.9 mmol) of sodium hydride dispersion (about 50% in mineral oil) and stirred at room temperature under argon for 90 minutes. Now, the reaction solution was partitioned between water and methylene chloride, the combined methylene chloride phases were dried over magnesium sulfate, filtered and concentrated in a water-jet vacuum. The thus obtained residue was chromatographed on silica gel with ether/methylene chloride (5:95). There were thus obtained 1.57 g (90% of theory) of 2-(3-benzyloxy-propyl)-5-bromo-pyridine in the form of a yellowish oil.

(b) 1.57 g (5.10 mmol) of 2-(3-benzyloxy-propyl)-5-bromo-pyridine, 1.6 ml (7.5 mmol) of hexamethyldistannate, 150 mg (0.129 mmol) of tetrakis-(triphenylphosphine)-palladium, 3 g of molecular seive (4 Å) and a few crystals of 2,6-di-tert-butyl-p-cresol were suspended in 18 ml of dioxan and the reaction mixture was stirred at 100° C. under argon for 2.5 hours. Thereupon, the reaction mixture was filtered, concentrated in a water-jet vacuum and the residue was chromatographed on silica gel with hexane/ethyl acetate (3:1). There were thus obtained 1.43 g (72% of theory) of 2-(3-benzyloxy-propyl)-5-trimethylstannanyl-pyridine in the form of a yellowish oil; MS: 392 (M+H)⁺.

(c) 1.43 g (3.66 mmol) of 2-(3-benzyloxy-propyl)-5-trimethylstannanyl-pyridine, 1.32 g (4.00 mmol) of tert-butyl 4-trifluoromethylsulfonyloxy-3,6-dihydro-2H-pyridine-1-carboxylate (Example 126 (c)), 0.477 g (11.3 mmol) of lithium chloride, 150 mg (0.129 mmol) of tetrakis-(triphenylphosphine)-palladium, 3 g of molecular seive (4 Å and a few crystals of 2,6-di-tert-butyl-p-cresol were suspended in 40 ml of 1,2-dimethoxyethane and the reaction mixture was stirred under reflux for 8 hours under argon. Thereupon, the mixture was filtered, concentrated in a water-jet vacuum and the residue was chromatographed on silica gel with methylene chloride/ether (1:1). There was thus obtained 0.903 g (60% of theory) of tert-butyl 6-(3-benzyloxy-propyl)-3′,6′-dihydro-2′H-[3,4′]bipyridinyl-1′-carboxylate in the form of a yellowish oil; MS: 409 (M+H)⁺.

(d) 0.115 g (0.28 mmol) of tert-butyl 6-(3-benzyloxy-propyl)-3′,6′-dihydro-2′H-[3,4′]bipyridinyl-1′-carboxylate were dissolved in 1 ml of 1,2-dimethoxyethane, treated with 1 ml of 1 molar borane-tetrahydrofuran solution and stirred at room temperature for 96 hours. Subsequently, while cooling with ice, 1.0 ml of 50% KOH solution in water followed by 1.0 ml of 30% hydrogen peroxide solution in water were added and the reaction mixture was heated under reflux for 2 hours. Now, the reaction solution was partitioned between water and methylene chloride and the combined methylene chloride phases were dried over magnesium sulpahte and concentrated. There were thus obtained 105 mg (88% of theory) of tert-butyl (3′RS,4′RS)-6-(3-benzyloxy-propyl)-3′-hydroxy-3′,4′,5′,6′-tetrahydro-2′H-[3,4′]bipyridine-1′-carboxylate in the form of a yellowish oil, which was used directly in the next step.

(e) In an analogous manner to that described in Example 125 (g), from tert-butyl (3′RS,4′RS)-6-(3-benzyloxy-propyl)-3′-hydroxy-3′,4′,5′,6′-tetrahydro-2′H-[3,4′]bipyridine-1′-carboxylate and 2-bromomethylnaphthalene there was obtained tert-butyl (3′RS,4′RS)-6-(3-benzyloxy-propyl)-3′-(naphthalen-2-ylmethoxy)-3′,4′,5′,6′-tetrahydro-2′H-[3,4′]bipyridine-1′-carboxylate in the form of a colorless oil; MS: 568 (M+H)⁺.

(f) In an analogous manner to that described in Example 125 (h), from tert-butyl (3′RS,4′RS)-6-(3-benzyloxy-propyl)-3′-(naphthalen-2-ylmethoxy)-3′,4′,5′,6′-tetrahydro-2′H-[3,4′]bipyridine-1′-carboxylate by cleavage of the BOC group by means of anhydrous zinc bromide there was obtained (3′RS,4′RS)-6-(3-benzyloxy-propyl)-3′-(naphthalen-2-ylmethoxy)-1′,2′,3′,4′,5′,6′-hexahydro-[3,4′]bipyridine in the form of a colorless oil; MS: 467 (M+H)⁺.

### Example 128

(a) 19 g of sodium hydride dispersion (50% in oil, 0.38 mol) were introduced portionwise at at maximum of 30° C. into 104 ml (1.0 mol) of benzyl alcohol dissolved in 175 ml of N,N-dimethyl-formamide and the mixture was stirred at room temperature for 2 hours. Thereupon, 46.4 g (0.183 mol) of 5-bromo-2-(2-trimethylsilyl)ethynyl-pyridine [J. Org. Chem. 53, 386 (1988)] in 50 ml of N,N-dimethylformamide were added dropwise within 10 minutes and the mixture was stirred at room temperature for 2 hours. Subsequently, the reaction mixture was poured into 1000 ml of saturated sodium hydrogen carbonate solution and extracted with ether. The combined ether phases were washed with saturated sodium chloride solution, dried over magnesium sulfate, filtered and concentrated in a water-jet vacuum. The residue thus obtained was chromatographed on silica gel with methylene chloride/ether (99:1). There were thus obtained 19.5 g (37% of theory) of (E)-2-(2-benzyloxy-vinyl)-5-bromo-pyridine in the form of a yellowish solid.

(b) 17.5 g (0.0603 mol) of (E)-2-(2-benzyloxy-vinyl)-5-bromo-pyridine were dissolved in 650 ml of toluene, treated with about 3 g of Raney-nickel (moist, washed with methanol and toluene) and stirred at room temperature in a hydrogen atmosphere for 72 hours. Within this period a further three similar amount of Raney-nickel were added. Thereupon, the mixture was filtered over a Dicalite pad, concentrated in a water-jet vacuum and the residue was chromatographed on silica gel with methylene chloride/ether (95:5). There were thus obtained 13.2 g (75.4% of theory) of 2-(2-benzyloxy-ethyl)-5-bromo-pyridine in the form of a reddish foam; MS: 292, 294 (M+H)⁺.

(c) In analogy to Example 127 (b)-(d), from 2-(2-benzyloxy-ethyl)-5-bromo-pyridine via 2-(2-benzyloxy-ethyl)-5-trimethylstannanyl-pyridine [yellowish oil, MS: 362 (M-CH₃)⁺] as well as tert-butyl 4-trifluoromethylsulfonyloxy-3,6-dihydro-2H-pyridine-1-carboxylate, and tert-butyl 6-(2-benzyloxy-ethyl)-3′,6′-dihydro-2′H-[3,4′]bipyridine-1′-carboxylate [colorless oil, MS: 395 (M+H)⁺], there was obtained tert-butyl (3′RS,4′RS)-6-(2-benzyloxy-ethyl)-3′-hydroxy-3′,4′,5′,6′-tetrahydro-2′H-[3,4′]-bipyridine-1′-carboxylate in the form of a colorless solid; MS: 413 (M+H)⁺.

(d) In an analogous manner to that described in Example 125 (g), from tert-butyl (3′RS,4′RS)-6-(2-benzyloxy-ethyl)-3′-hydroxy-3′,4′,5′,6′-tetrahydro-2′H-[3,4′]-bipyridine-1′-carboxylate and 2-bromomethylnaphthalene there was obtained tert-butyl (3′RS,4′RS)-6-(2-benzyloxy-ethyl)-3′-(naphthalen-2-ylmethoxy)-3′,4′,5′,6′-tetrahydro-2′H-[3,4′]bipyridine-1′-carboxylate in the form of an amorphous, colorless solid; MS: 553 (M+H)⁺.

(e) In an analogous manner to that described in Example 125 (h), from tert-butyl (3′RS,4′RS)-6-(2-benzyloxy-ethyl)-3′-(naphthalen-2-ylmethoxy)-3′,4′,5′,6′-tetrahydro-2′H-[3,4′]bipyridine-1′-carboxylate by cleavage of the BOC group by means of anhydrous zinc bromide there was obtained (3′RS,4′RS)-6-(2-benzyloxy-ethyl)-3′-(naphthalen-2-ylmethoxy)-1′,2′,3′,4′,5′,6′-hexahydro-[3,4′]bipyridine in the form of a beige gum; MS: 453 (M+H)⁺.

### Example 129

(a) In analogy to Example 127 (b)-(d), from 5-bromo-2-methylsulfanyl-pyrimidine [J. Chem. Soc. 1953, 3129] via 2-methylsulfanyl-5-trimethylstannanyl-pyrimidine (yellowish oil) as well as tert-butyl 4-trifluoromethylsulfonyloxy-3,6-dihydro-2H-pyridine-1-carboxylate, and tert-butyl 4-(2-methylsulfanyl-pyrimidin-5-yl)-3,6-dihydro-2H-pyridine-1-carboxylate (yellowish solid, MS: 308 (M+H)⁺), there was obtained tert-butyl (3RS,4RS)-3-hydroxy-4-(2-methylsulfanyl-pyrimidin-5-yl)-piperidine-1-carboxylate in the form of a yellowish, amorphous solid; MS: 326 (M+H)⁺.

(b) In an analogous manner to that described in Example 125 (g), from tert-butyl (3RS,4RS)-3-hydroxy-4-(2-methylsulfanyl-pyrimidin-5-yl)-piperidine-1-carboxylate and 2-bromomethyl-naphthalene there was obtained tert-butyl (3RS,4RS)-4-(2-methylsulfanyl-pyrimidin-5-yl)-3-(naphthalen-2-ylmethoxy)-piperidine-1-carboxylate in the form of an amorphous, yellowish solid; MS: 466 (M+H)⁺.

(c) 0.138 g (0.296 mmol) of tert-butyl (3RS,4RS)-4-(2-methylsulfanyl-pyrimidin-5-yl)-3-(naphthalen-2-ylmethoxy)-piperidine-1-carboxylate was dissolved in 5 ml of methylene chloride, treated with 0.113 g (about 0.46 mmol) of m-chloro-perbenzoic acid (about 70%), stirred at room temperature for 3 hours, treated with a further 0.050 g (about 0.20 mmol) of m-chloroperbenzoic acid and stirred at room temperature for a further 16 hours. Thereupon, the reaction mixture was partitioned between methylene chloride and saturated soda solution and the combined methylene chloride phases were dried over magnesium sulfate, filtered and concentrated in a water-jet vacuum. The thus obtained residue was crystallized from ether. There was thus obtained 0.102 g (69% of theory) of tert-butyl (3RS,4RS)-4-(2-methylsulfonyl-pyrimidin-5-yl)-3-(naphthalen-2-ylmethoxy)-piperidine-1-carboxylate in the form of a colorless solid; MS: 498 (M+H)⁺.

(d) 0.027 g (0.24 mmol) of potassium tert-butylate was placed in 1 ml of tetrahydrofuran and 0.038 g (0.22 mmol) of 3-benzyloxy-propanol dissolved in 0.5 ml of tetrahydrofuran was added dropwise at 0° C. After stirring at 0° C. for 15 minutes 0.098 g (0.20 mmol) of tert-butyl (3RS,4RS)-4-(2-methylsulfonyl-pyrimidin-5-yl)-3-(naphthalen-2-ylmethoxy)-piperidine-1-carboxylate dissolved in 1 ml of tetrahydrofuran was added dropwise at the same temperature and the reaction mixture was stirred at room temperature for 18 hours. Thereupon, the mixture was poured on to ice-water and extracted with ether. The combined ether phases were washed with saturated sodium chloride solution, dried over magnesium sulfate, filtered and concentrated in a water-jet vacuum. The residue thus obtained was chromatographed on silica gel with methylene chloride/methanol (95:5). There was thus obtained 0.076 g (66% of theory) of tert-butyl (3RS,4RS)-4-[2-(3-benzyloxy-propoxy)-pyrimidin-5-yl]-3-(naphthalen-2-ylmethoxy)-piperidine-1-carboxylate in the form of a colorless solid; MS: 585 (M+H)⁺.

(e) In an analogous manner to that described in Example 125 (h), from tert-butyl (3RS,4RS)-4-[2-(3-benzyloxy-propoxy)-pyrimidin-5-yl]-3-(naphthalen-2-ylmethoxy)-piperidine-1-carboxylate by cleavage of the BOC group by means of anhydrous zinc bromide there was obtained 2-(3-benzyloxy-propoxy)-5-[(3RS,4RS)-3-(naphthalen-2-ylmethoxy)-piperidin-4-yl]-pyrimidine in the form of a yellowish gum; MS: 485 (M+H)⁺.

### Example 130

(a) 0.90 g (2.18 mmol) of tert-butyl (3′RS,4′RS)-6-(2-benzyloxy-ethyl)-3′-hydroxy-3′,4′, 5′,6′-tetrahydro-2′H-[3,4′]-bipyridine-1′-carboxylate [Example 128 (c)] dissolved in 15 ml of tetrahydrofuran was treated with 0.3 ml of acetic acid and 250 mg of palladium-on-charcoal (10%) and the reaction mixture was stirred in a hydrogen atmosphere for 14 days. Subsequently, the mixture was filtered over a 0.8 mu. cellulose filter and concentrated in a water-jet vacuum. The residue thus obtained was partitioned between methylene chloride and saturated soda solution, the combined methylene chloride phases were dried over magnesium sulfate, filtered and concentrated in a water-jet vacuum. The crude product was subsquently chromatographed on silica gel with methylene chloride/methanol (95:5). There was thus obtained 0.555 g (80% of theory) of tert-butyl (3′RS,4′RS)-3′-hydroxy-6-(2-hydroxy-ethyl)-3′,4′,5′,6′-tetrahydro-2′H-[3,4′]bipyridine-1′-carboxylate in the form of a colorless gum; MS: 323 (M+H)⁺.

(b) 0.46 g (1.43 mmol) of tert-butyl (3′RS,4′RS)-3′-hydroxy-6-(2-hydroxy-ethyl)-3′,4′,5′,6′-tetrahydro-2′H-[3,4′]bipyridine-1′-carboxylate, 0.18 g (1.5 mmol) of 4-dimethylamino-pyridine and 0.32 ml (2.2 mmol) of triethylamine were placed in 5 ml of methylene chloride and treated with 0.55 g (1.7 mmol) of bromotriphenylmethane. After stirring at room temperature for 16 hours a further 0.32 ml (2.2 mmol) of triethylamine and 0.50 g (1.5 mmol) of bromotriphenylmethane were added and the mixture was stirred at room temperature for a futher hour. Thereupon, the reaction mixture was partitioned between methylene chloride and saturated soda solution and the combined methylene chloride phases were dried over magnesium sulfate, filtered and concentrated in a water-jet vacuum. The thus obtained residue was chromatographed on silica gel with methylene chloride/methanol (95:5). There was thus obtained 0.67 g (83% of theory) of tert-butyl (3′RS,4′RS)-3′-hydroxy-6-(2-trityloxy-ethyl)-3′,4′,5′,6′-tetrahydro-2′H-[3,4′]bipyridine-1′-carboxylate in the form of a colorless foam; MS: 566 (M+H)⁺.

(c) In an analogous manner to that described in Example 125 (g), from tert-butyl (3′RS,4′RS)-3′-hydroxy-6-(2-trityloxy-ethyl)-3′,4′,5′,6′-tetrahydro-2′H-[3,4′]bipyridine-1′-carboxylate and 2-bromomethylnaphthalene there was obtained tert-butyl (3′RS,4′RS)-3′-(naphthalen-2-ylmethoxy)-6-(2-trityloxy-ethyl)-3′,4′,5′,6′-tetrahydro-2′H-[3,4′]bipyridine-1′-carboxylate in the form of an amorphous, colorless solid; MS: 706 (M+H)⁺.

(d) 0.35 g (0.50 mmol) of tert-butyl (3′RS,4′RS)-3′-(naphthalen-2-ylmethoxy)-6-(2-trityloxy-ethyl)-3′,4′,5′,6′-tetrahydro-2′H-[3,4′]bipyridine-1′-carboxylate was dissolved in 8 ml of methylene chloride and treated rapidly at room temperature with a solution of 240 mg of trifluoroacetic acid and 440 mg of trifluoroacetic anhydride in 2 ml of methylene chloride and the reaction mixture was stirred for 50 seconds. Thereupon, while cooling with ice, 2.2 ml of triethylamine followed by 3 ml of methanol were added and the mixture was stirred without cooling for 10 minutes. Thereupon, the reaction mixture was partitioned between methylene chloride and saturated soda solution, the combined methylene chloride phases were dried over magnesium sulfate, filtered and concentrated in a water-jet vacuum. The residue thus obtained was chromatographed on silica gel with methylene chloride/methanol (95:5). There was thus obtained 0.189 g (82% of theory) of tert-butyl (3′RS,4′RS)-6-(2-hydroxy-ethyl)-3′-(naphthalen-2-ylmethoxy)-3′,4′,5′,6′-tetrahydro-2′H-[3,4′]bipyridine-1′-carboxylate in the form of an amorphous, orange solid; MS: 463 (M+H)⁺.

(e) 0.060 g (0.129 mmol) of tert-butyl (3′RS,4′RS)-6-(2-hydroxy-ethyl)-3′-(naphthalen-2-ylmethoxy)-3′,4′,5′,6′-tetrahydro-2′H-[3,4′]bipyridine-1′-carboxylate and 0.022 g (0.129 mmol) of 2-chloro-benzothiazole were dissolved in 0.5 ml of N,N-dimethylformamide and treated with 0.008 g (about 50% in mineral oil, about 0.17 mmol) of sodium hydride and the reaction mixture was stirred at room temperature for 4.5 hours. Thereupon, the mixture was treated with ice-water and extracted with ether. The combined ether phases were dried over magnesium sulfate, filtered and concentrated in a water-jet vacuum. The thus obtained residue was chromatographed on silica gel with methylene chloride/ether (1:1). There was thus obtained 0.053 g (70% of theory) of tert-butyl (3′RS,4′RS)-3′-(naphthalen-2-ylmethoxy)-6-vinyl-3′,4′,5′,6′-tetrahydro-2′H-[3,4′]bipyridine-1′-carboxylate in the form of an amorphous, colorless solid; MS: 446 (M+H)⁺.

(f) 0.041 g (0.091 mmol) of tert-butyl (3′RS,4′RS)-3′-(naphthalen-2-ylmethoxy)-6-vinyl-3′,4′,5′,6′-tetrahydro-2′H-[3,4′]bipyridine-1′-carboxylate and 0.054 g (0.32 mmol) of 2-mercaptobenzothiazole were dissolved in 0.5 ml of acetonitrile, treated with 0.2 ml (0.12 mmol) of 0.6M sodium methylate solution in methanol and stirred at 80° C. for 3.5 hours. Thereupon, the reaction mixture was concentrated and the residue was chromatographed on silica gel with ethyl acetate/hexane (3:2). There was thus obtained 0.040 g (72% of theory) of tert-butyl (3′RS,4′RS)-3′-(naphthalen-2-ylmethoxy)-6-[2-(2-thioxo-benzothiazol-3-yl)-ethyl]-3′,4′,5′,6′-tetrahydro-2′H-[3,4′]bipyridine-1′-carboxylate in the form of an amorphous, yellowish solid; MS: 612 (M+H)⁺.

(g) In an analogous manner to that described in Example 125 (h), from tert-butyl (3′RS,4′RS)-3′-(naphthalen-2-ylmethoxy)-6-[2-(2-thioxo-benzothiazol-3-yl)-ethyl]-3′,4′,5′,6′-tetrahydro-2′H-[3,4′]bipyridine-1′-carboxylate by cleavage of the BOC group by means of anhydrous zinc bromide there was obtained 3-[2-[(3′RS,4′RS)-3′-(naphthalen-2-ylmethoxy)-1′,2′,3′,4′,5′,6′-hexahydro-[3,4′]bipyridin-6-yl]-ethyl]-3H-benzothiazole-2-thione in the form of an amorphous, colorless solid; MS: 512 (M+H)⁺.

### Example 131

5 (a) 0.15 g (0.56 mmol) of 5-bromomethyl-3-(2-chloro-phenyl)-[1,2,4]oxadiazole [Example 124], 0.21 g of potassium carbonate and 0.15 g of sodium hydrogen carbonate were stirred at 65° C. for 42 hours in a mixture of 4.5 ml of tetrahydrofuran and 1.0 ml of water. Thereupon, the reaction mixture was cooled, diluted with water and extracted with ether. The combined ether phases were dried over magnesium sulfate, filtered and concentrated in a water-jet vacuum. The thus obtained residue was chromatographed on silica gel with methylene chloride/ether (9:1). There was thus obtained 0.041 g (35% of theory) of [3-(2-chloro-phenyl)-[1,2,4]oxadiazol-5-yl]-methanol in the form of a colorless solid; MS: 210 (M)⁺.

(b) 0.040 g (0.19 mmol) of [3-(2-chloro-phenyl)-[11,2,4]oxadiazol-5-yl]-methanol and 0.094 g (0.19 mmol) of tert-butyl (3RS,4RS)-4-(2-methylsulfonyl-pyrimidin-5-yl)-3-(naphthalen-2-ylmethoxy)-piperidine-1-carboxylate [Example 129 (c)] were dissolved in 0.5 ml of N,N-dimethylformamide, treated while cooling with ice with 0.0095 g (about 50% in mineral oil, about 0.20 mmol) of sodium hydride and the reaction mixture was stirred at room temperature for 1 hour. Thereupon, the mixture was treated with ice-water and extracted with ether. The combined ether phases were dried over magnesium sulfate, filtered and concentrated in a water-jet vacuum. The residue thus obtained was chromatographed on silica gel with ether. There was thus obtained 0.112 g (94% of theory) of tert-butyl (3RS,4RS)-4-[2-[3-(2-chlorophenyl)-[1,2,4]oxadiazol-5-ylmethoxy]-pyrimidin-5-yl]-3-(naphthalen-2-ylmethoxy)-piperidine-1-carboxylate in the form of an amorphous, reddish gum; MS: 628 (M+H)⁺.

(c) In an analogous manner to that described in Example 125 (h), from tert-butyl (3RS,4RS)-4-[2-[3-(2-chlorophenyl)-[1,2,4]oxadiazol-5-ylmethoxy]-pyrimidin-5-yl]-3-(naphthalen-2-ylmethoxy)-piperidine-1-carboxylate by cleavage of the BOC group by means of anhydrous zinc bromide there was obtained 2-[3-(2-chloro-phenyl)-[1,2,4]oxadiazol-5-ylmethoxy]-5-[(3RS,4RS)-3-(naphthalen-2-ylmethoxy)-piperidin-4-yl]-pyrimidine in the form of an amorphous yellowish solid; MS: 528 (M+H)⁺.

### Example 132

(a) 2.3 g (10 mmol) of 3-benzyloxy-1-bromo-propane and 0.76 g (10 mmol) of thiourea were heated under reflux in 5.0 ml of ethanol for 3.5 hours. Thereupon, the mixture was cooled to room temperature, treated with 0.6 g (15 mmol) of sodium hydroxide in 6.0 ml of water and stirred under argon for a further 3 hours. Thereupon, the mixture was acidified with dilute hydrochloric acid and extracted with ether. The combined ether phases were dried over magnesium sulfate, filtered and concentrated in a water-jet vacuum. The thus obtained residue was chromatographed on silica gel with hexane/methylene chloride (1:1). There were thus obtained 1.5 g (83% of theory) of 3-benzyloxy-propane-1-thiol as a colorless liquid.

(b) In an analogous manner to that described in Example 131 (b), from 3-benzyloxy-propane-1-thiol and tert-butyl (3RS,4RS)-4-(2-methylsulfonyl-pyrimidin-5-yl)-3-(naphthalen-2-ylmethoxy)-piperidine-1-carboxylate [Example 129 (c)] there was obtained tert-butyl (3RS,4RS)-4-[2-(3-benzyloxy-propylsulfanyl)-pyrimidin-5-yl]-3-(naphthalen-2-ylmethoxy)-piperidine-1-carboxylate in the form of an amorphous, pink solid; MS: 600 (M+H)⁺.

(c) In an analogous manner to that described in Example 125 (h), from tert-butyl (3RS,4RS)-4-[2-(3-benzyloxy-propylsulfanyl)-pyrimidin-5-yl]-3-(naphthalen-2-ylmethoxy)-piperidine-1-carboxylate by cleavage of the BOC group by means of anhydrous zinc bromide there was obtained 2-(3-benzyloxy-propylsulfanyl)-5-[(3RS,4RS)-3-(naphthalen-2-ylmethoxy)-piperidin-4-yl]-pyrimidine in in the form of an amorphous, colorless solid; MS: 500 (M+H)⁺.

### Example 133

(a) 0.50 g (2.2 mmol) of 3-benzyloxy-1-bromo-propane was treated with 2.5 ml of 30 percent methylamine solution in ethanol and stirred at 60° C. in a sealed vessel for 10 hours. Thereupon, the mixture was treated with ice-water and extracted with ether. The combined ether phases were dried over magnesium sulfate, filtered and concentrated in a water-jet vacuum. The thus obtained residue was chromatographed on silica gel with methylene chloride/methanol (9:1). There was thus obtained 0.16 g (41% of theory) of (3-benzyloxy-propyl)-methyl-amine as a colorless oil.

(b) 0.15 g (0.84 mmol) of (3-benzyloxy-propyl)-methyl-amine and 0.060 g (0.12 mmol) of tert-butyl (3RS,4RS)-4-(2-methyl-sulfonyl-pyrimidin-5-yl)-3-(naphthalen-2-ylmethoxy)-piperidine-1-carboxylate [Example 129 (c)] were stirred at 80° C. in 1.5 ml of triethylamine for 18 hours under argon. Thereupon, the mixture was treated with ice-water and extracted with ether. The combined ether phases were dried over magnesium sulfate, filtered and concentrated in a water-jet vacuum. The thus obtained residue was chromatographed on silica gel with methylene chloride/ethyl acetate (1:1). There was thus obtained 0.070 g (97% of theory) of tert-butyl (3RS,4RS)-4-[2-[(3-benzyloxy-propyl)-methyl-amino]-pyrimidin-5-yl]-3-(naphthalen-2-ylmethoxy)-piperidine-1-carboxylate in the form of a brownish gum; MS: 598 (M+H)⁺.

(c) In an analogous manner to that described in Example 125 (h), from tert-butyl (3RS,4RS)-4-[2-[(3-benzyloxy-propyl)-methyl-amino]-pyrimidin-5-yl]-3-(naphthalen-2-ylmethoxy)-piperidine-1-carboxylate by cleavage of BOC group by means of anhydrous zinc bromide there was obtained (3-benzyloxy-propyl)-methyl-[5-[(3RS,4RS)-3-(naphthalen-2-ylmethoxy)-piperidin-4-yl]-pyrimidin-2-yl]-amine in the form of an amorphous colorless solid; MS: 498 (M+H)⁺.

### Example 134

(a) 1.15 g (5.0 mmol) of 3-benzyloxy-1-bromo-propane and 1.02 g (5.5 mmol) of potassium phthalimide were stirred at 70-80° C. in 10 ml of N,N-dimethylformamide for 2 hours. Thereupon, the mixture was treated with ice-water and the precipitate which thereupon formed was filtered off, washed with water and dried over phosphorus pentoxide in a water-jet vacuum. There were thus obtained 1.4 g (95% of theory) of 2-(3-benzyloxy-propyl)-isoindole-1,3-dione as a colorless solid.

(b) 1.4 g (4.7 mmol) of 2-(3-benzyloxy-propyl)-isoindole-1,3-dione and 0.9 ml of hydrazine monohydrate were stirred at 100° C. in 10 ml of absolute ethanol under argon for 2 hours. After cooling the mixture was treated with ether, filtered and the filtrate was concentrated. There was thus obtained 0.75 g (96% of theory) of 3-benzyloxy-propylamine as a light yellowish oil.

(c) In an analogous manner to that described in Example 133 (b), from 3-benzyloxy-propylamine and tert-butyl (3RS,4RS)-4-(2-methylsulfonyl-pyrimidin-5-yl)-3-(naphthalen-2-ylmethoxy)-piperidine-1-carboxylate [Example 129 (c)] there was obtained tert-butyl (3RS,4RS)-4-[2-(3-benzyloxy-propylamino)-pyrimidin-5-yl]-3-(naphthalen-2-ylmethoxy)-piperidine-1-carboxylate in the form of a brownish gum; MS: 583 (M+H)⁺.

(d) In an analogous manner to that described in Example 125 (h), from tert-butyl (3RS,4RS)-4-[2-(3-benzyloxy-propylamino)-pyrimidin-5-yl]-3-(naphthalen-2-ylmethoxy)-piperidine-1-carboxylate by cleavage of the BOC group by means of anhydrous zinc bromide there was obtained (3-benzyloxy-propyl)-{5-[(3RS,4RS)-3-(naphthalen-2-ylmethoxy)-piperidin-4-yl]-pyrimidin-2-yl}-amine in the form of a brownish gum; MS: 483 (M+H)⁺.

### Example 135

The following compounds were obtained in an analogous manner to that described in Example 125 (h) by cleavage of the BOC group by means of anhydrous zinc bromide:

1)—From tert-butyl (3RS,4RS)-4-(2-methylsulfanyl-pyrimidin-5-yl)-3-(naphthalen-2-ylmethoxy)-piperidine-1-carboxylate [Example 129 (b)], 2-methylsulfanyl-5-[(3RS,4RS)-3-(naphthalen-2-ylmethoxy)-piperidin-4-yl]-pyrimidine in the form of an amorphous, yellowish solid; MS: 366 (M+H)⁺;

2)—from tert-butyl (3′RS,4′RS)-6-(3-benzyloxy-propyl)-3′-(naphthalen-2-ylmethoxy)-1-oxy-3′,4′,5′,6′-tetrahydro-2′H-[3,4′]bipyridine-1′-carboxylate, (3′RS,4′RS)-6-(3-benzyloxy-propyl)-3′-(naphthalen-2-ylmethoxy)-1′,2′,3′,4′,5′,6′-hexahydro-[3,4′]bipyridine-1-oxide in the form of a colorless oil; MS: 483 (M+H)⁺;

3)—from tert-butyl (3′RS,4′RS)-3′-(naphthalen-2-ylmethoxy)-6-(3-phenyl-propyl)-1-oxy-1 3′,4′,5′,6′-tetrahydro-2′H-[3,4′]bipyridine-1′-carboxylate, (3′RS,4′RS)-3′-(naphthalen-2-ylmethoxy)-6-(3-phenyl-propyl)-1′,2′,3′,4′,5′,6′-hexahydro-[3,4′]bipyridine-1-oxide in the form of an amorphous, yellowish solid; MS: 453 (M+H)⁺;

4)—from tert-butyl (3RS,4RS)-3-(naphthalen-2-ylmethoxy)-4-[2-(4-phenyl-butylamino)-pyrimidin-5-yl]-piperidine-1-carboxylate, [5-[(3RS,4RS)-3-(naphthalen-2-ylmethoxy)-piperidin-4-yl]pyrimidin-2-yl]-(4-phenyl-butyl)-amine in the form of an amorphous, reddish solid; MS: 467 (M+H)⁺;

5)—from tert-butyl (3RS,4RS)-4-{2-[methyl-(4-phenyl-butyl)-amino]-pyrimidin-5-yl}-3-(naphthalen-2-ylmethoxy)-piperidine-1-carboxylate, methyl-{5-[(3RS,4RS)-3-(naphthalen-2-ylmethoxy)-piperidin-4-yl]-pyrimidin-2-yl}-(4-phenyl-butyl)-amine in the form of an amorphous, colorless solid; MS: 481 (M+H)⁺.

The BOC derivatives used as starting materials were prepared as follows:

(b) 0.074 g (0.13 mmol) of tert-butyl(3′RS,4′RS)-6-(3-benzyloxy-propyl)-3′-(naphthalen-2-ylmethoxy)-3′,4′,5′,6′-tetrahydro-2′H-[3,4′]bipyridine-1′-carboxylate [Example 127 (e)] was dissolved in 1.5 ml of methylene chloride, treated with 0.046 g (about 0.19 mmol) of m-chloroperbenzoic acid (about 70%) and stirred at room temperature for 30 minutes. Thereupon, the reaction mixture was partitioned between methylene chloride and saturated soda solution and the combined methylene chloride phases are dried over magnesium sulfate, filtered and concentrated in a water-jet vacuum. The thus obtained residue was chromatographed on silica gel with methylene chloride/methanol (95:5). There was thus obtained 0.032 g (42% of theory) of tert-butyl (3′RS,4′RS)-6-(3-benzyloxy-propyl)-3′-(naphthalen-2-ylmethoxy)-1-oxy-3′,4′,5′,6′-tetrahydro-2′H-[3,4′]bipyridine-1′-carboxylate in the form of an amorphous, colorless solid; MS: 584 (M+H)⁺.

(c) In an analogous manner to that described in Example 135 (b), from tert-butyl (3′RS,4′RS)-3′-(naphthalen-2-ylmethoxy)-6-(3-phenyl-propyl)-3′,4′,5′,6′-tetrahydro-2′H-[3,4′]bipyridine-1′-carboxylate [Example 126 (g)] there was obtained tert-butyl (3′RS,4′RS)-3′-(naphthalen-2-ylmethoxy)-6-(3-phenyl-propyl)-1-oxy-3′,4′,5′,6′-tetrahydro-2′H-[3,4′]bipyridine-1′-carboxylate in the form of a colorless oil; MS: 553 (M+H)⁺.

(d) In an analogous manner to that described in Example 133 (b), from tert-butyl (3RS,4RS)-4-(2-methylsulfonyl-pyrimidin-5-yl)-3-(naphthalen-2-ylmethoxy)-piperidine-1-carboxylate and 4-phenylbutylamine there was obtained tert-butyl (3RS,4RS)-3-(naphthalen-2-ylmethoxy)-4-[2-(4-phenyl-butylamino)-pyrimidin-5-yl]-piperidine-1-carboxylate in the form of an amorphous, colorless solid; MS: 568 (M+H)⁺.

(e) 0.058 g (0.10 mmol) of tert-butyl (3RS,4RS)-3-(naphthalen-2-ylmethoxy)-4-[2-(4-phenyl-butylamino)-pyrimidin-5-yl]-piperidine-1-carboxylate was dissolved in 1.0 ml of N,N-dimethylformamide and treated at 0° C. with 0.08 ml (1.3 mmol) of methyl iodide followed by 0.010 g (about 0.2 mmol) of sodium hydride dispersion (about 50% in mineral oil) and stirred at room temperature under argon for 90 minutes. Now, the reaction solution was partitioned between water and ether and the combined ether phases were dried over magnesium sulfate, filtered and concentrated in a water-jet vacuum. The residue thus obtained was chromatographed on silica gel with hexane/ethyl acetate (1:1). There was thus obtained 0.021 g (35% of theory) of tert-butyl (3RS,4RS)-4-{2-[methyl-(4-phenyl-butyl)-amino]-pyrimidin-5-yl}-3-(naphthalen-2-ylmethoxy)-piperidine-1-carboxylate in the form of an amorphous, colorless solid; MS: 582 (M+H)⁺.

### Example 136

(a) A solution of 40 mg (0.080 mmol) of tert-butyl (3RS,4RS)-4-(2-methylsulfonyl-pyrimidin-5-yl)-3-(naphthalen-2-ylmethoxy)-piperidine-1-carboxylate [Example 129 (c)] in 1.1 ml 2M hydrogen chloride in methanol was stirred at room temperature for 5 hours. Subsequently, the reaction solution was partitioned between saturated sodium carbonate solution and methylene chloride and the combined methylene phases were dried over magnesium sulfate and concentrated. There were thus obtained 33 mg (95% of theory) of 2-methylsulfonyl-5-[(3RS,4RS)-3-(naphthalen-2-ylmethoxy)-piperidin-4-yl]-pyrimidine hydrochloride in the form of a colorless solid; MS: 398 (M+H)⁺.

(b) The following compounds were obtained by reacting 2-methylsulfonyl-5-[(3RS,4RS)-3-(naphthalen-2-ylmethoxy)-piperidin-4-yl]pyrimidine hydrochloride with alcohols in an analogous manner to that described in Example 131 (b), but using 2 equivalents of sodium hydride:

1)—With (E)-3-phenyl-2-propen-1-ol, (E)-5-[(3RS,4RS)-3-(naphthalen-2-ylmethoxy)-piperidin-4-yl]-2-(3-phenyl-allyloxy)-pyrimidine in the form of an amorphous, yellowish solid; MS: 452 (M+H)⁺;

2)—with (E)-2-methyl-3-phenyl-2-propen-1-ol, (E)-2-(2-methyl-3-phenyl-allyloxy)-5-[(3RS,4RS)-3-(naphthalen-2-ylmethoxy)-piperidin-4-yl]-pyrimidine in the form of an amorphous, yellowish solid; MS: 466 (M+H)⁺.

### Example 137

The following compounds were obtained by reacting tert-butyl (3RS,4RS)-4-(2-methylsulfonyl-pyrimidin-5-yl)-3-(naphthalen-2-ylmethoxy)-piperidine-1-carboxylate [Example 129 (c)]with alcohols and phenols in an analogous manner to that described in Example 131 (b) and subsequently cleaving off the BOC group by means of 2M hydrogen chloride in methanol as described in Example 136 (a):

1)—With 3-hydroxy-biphenyl, 2-(biphenyl-3-yloxy)-5-[(3RS,4RS)-3-(naphthalen-2-ylmethoxy)-piperidin-4-yl]-pyrimidine in the form of an amorphous, colorless solid; MS: 488 (M+H)⁺;

2)—with 3-phenoxy-benzyl alcohol, 5-[(3RS,4RS)-3-(naphthalen-2-ylmethoxy)-piperidin-4-yl]-2-(3-phenoxy-benzyloxy)-pyrimidine in the form of an amorphous, brownish solid; MS: 519 (M+H)⁺;

3)—with 4-phenoxy-phenol, 5-[(3RS,4RS)-3-(naphthalen-2-ylmethoxy)-piperidin-4-yl]-2-(4-phenoxy-phenoxy)-pyrimidine in the form of an amorphous, brownish solid; MS: 504 (M+H)⁺, as well as 2-methoxy-5-[(3RS,4RS)-3-(naphthalen-2-ylmethoxy)-piperidin-4-yl]-pyrimidine in the form of an amorphous, brownish gum; MS: 349 (M+H)⁺;

4)—with 4-hydroxy-biphenyl, 2-(biphenyl-4-yloxy)-5-[(3RS,4RS)-3-(naphthalen-2-ylmethoxy)-piperidin-4-yl]-pyrimidine in the form of an amorphous, colorless solid; MS: 488 (M+H)⁺.

5)—with 3-phenyl-2-propyn-1-ol, 5-[(3RS,4RS)-3-(naphthalen-2-ylmethoxy)-piperidin-4-yl]-2-(3-phenyl-prop-2-ynyloxy)-pyrimidine in the form of an amorphous, brownish solid; MS: 451 (M+H)⁺;

6)—with 2-hydroxymethyl-2,3-dihydro-benzo[1,4]dioxin, 2-(2RS and 2SR)-(2,3-dihydro-benzo[1,4]dioxin-2-ylmethoxy)-5-[(3RS,4RS)-3-(naphthalen-2-ylmethoxy)-piperidin-4-yl]-pyrimidine in the form of a colorless solid; MS: 484 (M+H)⁺;

7)—with 4-biphenyl-ethanol [Chemische Berichte 85, 897 (1952)], 2-(2-biphenyl-4-yl-ethoxy)-5-[(3RS,4RS)-3-(naphthalen-2-ylmethoxy)-piperidin-4-yl]-pyrimidine in the form of an amorphous, yellowish gum; MS: 517 (M+H)⁺;

8)—with 4-phenoxy-benzyl alcohol, 5-[(3RS,4RS)-3-(naphthalen-2-ylmethoxy)-piperidin-4-yl]-1-(4-phenoxy-benzyl)-1H-pyrimidin-2-one in the form of an amorphous, colorless solid; MS: 518 (M+H)⁺;

9)—with 4-biphenyl-methanol, 2-(biphenyl-4-ylmethoxy)-5-[(3RS,4RS)-3-(naphthalen-2-ylmethoxy)-piperidin-4-yl]-pyrimidine in the form of a yellowish gum; MS: 503 (M+H)⁺;

10)—with (1-(4-chloro-phenyl)-cyclopentyl]-methanol, 2-[[1-(4-chloro-phenyl)-cyclopentyl]-methoxy]-5-[(3RS,4RS)-3-(naphthalen-2-ylmethoxy)-piperidin-4-yl)-pyrimidine in the form of a colorless solid; MS: 528 (M+H)⁺;

11)—with 2-naphthalene-methanol, 2-(naphthalen-2-ylmethoxy)-5-[(3RS,4RS)-3-(naphthalen-2-ylmethoxy)-piperidin-4-yl]-pyrimidine in the form of an amorphous, yellowish solid; MS: 476 (M+H)⁺;

12)—with 2-naphthalene-ethanol, 2-(2-naphthalen-2-yl-ethoxy)-5-[(3RS,4RS)-3-(naphthalen-2-ylmethoxy)-piperidin-4-yl]-pyrimidine in the form of an amorphous, brownish solid; MS: 490 (M+H)⁺;

13)—with 2-(4-bromophenyl)-ethanol, 2-[2-(4-bromo-phenyl)-ethoxy]-5-[(3RS,4RS)-3-(naphthalen-2-ylmethoxy)-piperidin-4-yl]-pyrimidine in the form of an amorphous, brownish solid; MS: 518, 520 (M+H)⁺;

14)—with 2-(2-chloro-phenoxy)-ethanol, 2-[2-(2-chloro-phenoxy)-ethoxy]-5-[(3RS,4RS)-3-(naphthalen-2-ylmethoxy)-piperidin-4-yl]-pyrimidine in the form of an amorphous, brownish solid; MS: 490 (M+H)⁺;

15)—with 2-benzyloxy-ethanol, 2-(2-benzyloxy-ethoxy)-5-[(3RS,4RS)-3-(naphthalen-2-ylmethoxy)-piperidin-4-yl]-pyrimidine in the form of an amorphous, yellowish solid; MS: 470 (M+H)⁺;

16)—with 3-cyclohexyl-propanol, 2-(3-cyclohexyl-propoxy)-5-[(3RS,4RS)-3-(naphthalen-2-ylmethoxy)-piperidin-4-yl]-pyrimidine in the form of an amorphous, brownish solid; MS: 461 (M+H)⁺;

17)—with 3-(6-methyl-pyridin-2-yl)-propanol, 2-[3-(6-methyl-pyridin-2-yl)-propoxy]-5-[(3RS,4RS)-3-(naphthalen-2-ylmethoxy)-piperidin-4-yl]-pyrimidine in the form of an amorphous, brownish solid; MS: 469 (M+H)⁺;

18)—with 2-cyclohexyloxy-ethanol, 2-(2-cyclohexyloxy-ethoxy)-5-[(3RS,4RS)-3-(naphthalen-2-ylmethoxy)-piperidin-4-yl]-pyrimidine in the form of an amorphous, yellowish solid; MS: 462 (M+H)⁺;

19)—with 2-(phenylthio)-ethanol, 5-[(3RS,4RS)-3-(naphthalen-2-ylmethoxy)-piperidin-4-yl]-2-(2-phenylsulfanyl-ethoxy)-pyrimidine in the form of an amorphous, yellowish solid; MS: 472 (M+H)⁺;

20)—with 2-(5-methyl-2-phenyloxazol-4-yl)-ethanol (acquired from Maybridge Chemical Company), 2-[2-(5-methyl-2-phenyl-oxazol-4-yl)-ethoxy]-5-[(3RS,4RS)-3-(naphthalen-2-ylmethoxy)-piperidin-4-yl]-pyrimidine in the form of an amorphous, brownish solid; MS: 522 (M+H)⁺;

21)—with 2-cyclohexyl-ethanol, 2-(2-cyclohexyl-ethoxy)-5-[(3RS,4RS)-3-(naphthalen-2-ylmethoxy)-piperidin-4-yl]-pyrimidine in the form of an amorphous, colorless solid; MS: 447 (M+H)⁺;

22)—with (RS)-4-(2-hydroxy-ethyl)-5-methyl-2-phenyl-2,4-dihydro-pyrazol-3-one (acquired from Maybridge Chemical Company), a mixture of (RS)- and (SR)-5-methyl-4-[2-[5-[(3RS,4RS)-3-(naphthalen-2-ylmethoxy)-piperidin-4-yl]-pyrimidin-2-yloxy]-ethyl]-2-phenyl-2,4-dihydro-pyrazol-3-one in the form of an amorphous, colorless solid; MS: 536 (M+H)⁺.

### Example 138

0.045 g (0.082 mmol) of tert-butyl (3′RS,4′RS)-3′-(naphthalen-2-ylmethoxy)-6-(3-phenyl-propyl)-1-oxy-3′,4′,5′,6′-tetrahydro-2′H-[3,4′]bipyridine-1′-carboxylate [Example 135 (c)] was dissolved in 1.5 ml of N,N-dimethylformamide, treated with 0.10 ml (0.8 mmol) of trifluoroacetic anhydride and stirred at room temperature for 2 hours. Thereupon, the reaction mixture was partitioned between methylene chloride and saturated bicarbonate solution and the combined methylene chloride phases were dried over magnesium sulfate, filtered and concentrated in a water-jet vacuum. The thus obtained residue was chromatographed on silica gel with methylene chloride/methanol (95:5). There was thus obtained 0.019 g (52% of theory) of (3′RS,4′RS)-3′-(naphthalen-2-ylmethoxy)-6-(3-phenyl-propyl)-1′,2′,3′,4′,5′,6′-hexahydro-[3,4′]bipyridin-4-ol in the form of an amorphous, colorless solid; MS: 453 (M+H)⁺.

### Example 139

The following compounds were obtained by reacting tert-butyl (3RS,4RS)-3-(1,4-dimethoxy-naphthalen-2-ylmethoxy)-4-(2-methylsulfonyl-pyrimidin-5-yl)-piperidine-1-carboxylate with alcohols in an analogous manner to that described in Example 131 (b) and subsequently cleaving off the BOC group by means of anhydrous zinc bromide in methylene chloride as described in Example 125 (h):

1)—With 3-phenyl-2-propyn-1-ol, 5-[(3RS,4RS)-3-(1,4-dimethoxy-naphthalen-2-ylmethoxy)-piperidin-4-yl]-2-(3-phenyl-prop-2-ynyloxy)-pyrimidine in the form of an amorphous, yellowish solid; MS: 510 (M+H)⁺;

2)—with 3-cyclohexyl-propanol, 2-(3-cyclohexyl-propoxy)-5-[(3RS,4RS)-3-(1,4-dimethoxy-naphthalen-2-ylmethoxy)-piperidin-4-yl]-pyrimidine in the form of an amorphous, brownish solid; MS: 521 (M+H)⁺;

3)—with 4-cyclohexyl-butanol, 2-(4-cyclohexyl-butoxy)-5-[(3RS,4RS)-3-(1,4-dimethoxy-naphthalen-2-ylmethoxy)-piperidin-4-yl]-pyrimidine in the form of an amorphous, yellowish solid; MS: 535 (M+H)⁺;

4)—with 2-indan-2-yl-ethanol [J. Am. Chem. Soc. 87, 1297 (1965)], 5-[(3RS,4RS)-3-(1,4-dimethoxy-naphthalen-2-ylmethoxy)-piperidin-4-yl]-2-(2-indan-2-yl-ethoxy)-pyrimidine in the form of an amorphous, yellowish solid; MS: 540 (M+H)⁺;

5)—with 3-(2-methoxy-benzyloxy)-propan-1-ol (prepared by alkylating propylene glycol in a large excess with 2-methoxy-benzyl chloride using sodium hydride in N,N-dimethylformamide), 5-[(3RS,4RS)-3-(1,4-dimethoxy-naphthalen-2-ylmethoxy)-piperidin-4-yl]-2-[3-(2-methoxy-benzyloxy)propoxy]-pyrimidine in the form of an amorphous, colorless solid; MS: 574 (M+H)⁺;

6)—with (E)-4-phenyl-but-3-en-1-ol [Example 122 (b)], 5-[(3RS,4RS)-3-(1,4-dimethoxy-naphthalen-2-ylmethoxy)-piperidin-4-yl]-2-[(E)-4-phenyl-but-3-enyloxy]-pyrimidine in the form of an amorphous, colorless solid; MS: 526 (M+H)⁺;

7)—with 2-(5-phenyl-pyridin-2-yl)-ethanol [Example 139 (b)], 5-[(3RS,4RS)-3-(1,4-s dimethoxy-naphthalen-2-ylmethoxy)-piperidin-4-yl]-2-[2-(5-phenyl-pyridin-2-yl)-ethoxy]-pyrimidine in the form of an amorphous, colorless solid; MS: 576 (M)⁺;

8)—with 5-phenyl-4-pentyn-1-ol, 5-[(3RS,4RS)-3-(1,4-dimethoxy-naphthalen-2-ylmethoxy)-piperidin-4-yl]-2-(5-phenyl-pent-4-ynyloxy)-pyrimidine in the form of an amorphous, orange solid; MS: 537 (M)⁺.

The following compounds were obtained by reacting tert-butyl (3RS,4RS)-3-(1,4-dimethoxy-naphthalen-2-ylmethoxy)-4-(2-methylsulfonyl-pyrimidin-5-yl)-piperidine-1-carboxylate with amines in an analogous manner to that described in Example 133 (b) and subsequently cleaving off the BOC group by means of anhydrous zinc bromide in methylene chloride as described in Example 125 (h):

9)—With 4-phenylbutylamine, {5-[(3RS,4RS)-3-(1,4-dimethoxy-naphthalen-2-ylmethoxy)-piperidin-4-yl]-pyrimidin-2-yl}-(4-phenyl-butyl)-amine in the form of an amorphous, orange solid; MS: 527 (M+H)⁺;

10)—with 2-(5-phenyl-pyridin-2-yl)-ethylamine [Example 139 (d)], {5-[(3RS,4RS)-3-(1,4-dimethoxy-naphthalen-2-ylmethoxy)-piperidin-4-yl]-pyrimidin-2-yl}-[2-(5-phenyl-pyridin-2-yl)-ethyl]-amine in the form of an amorphous, colorless solid; MS: 576 (M+H)⁺;

11)—with 3-methoxy-benzylamine, {5-[(3RS,4RS)-3-(1,4-dimethoxy-naphthalen-2-ylmethoxy)-piperidin-4-yl]-pyrimidin-2-yl}-(3-methoxy-benzyl)-amine in the form of an amorphous, colorless solid; MS: 514 (M)⁺;

12)—with 4-methoxy-benzylamine, {5-[(3RS,4RS)-3-(1,4-dimethoxy-naphthalen-2-ylmethoxy)-piperidin-4-yl]-pyrimidin-2-yl}-(4-methoxy-benzyl)-amine in the form of an amorphous, yellowish solid; MS: 514 (M)⁺;

13)—with 3-bromo-benzylamine, (3-bromo-benzyl)-{5-[(3RS,4RS)-3-(1,4-dimethoxy-naphthalen-2-ylmethoxy)-piperidin-4-yl]-pyrimidin-2-yl}-amine in the form of an amorphous, colorless solid; MS: 562, 564 (M)⁺;

14)—with 3-methyl-benzylamine, {5-[(3RS,4RS)-3-(1,4-dimethoxy-naphthalen-2-ylmethoxy)-piperidin-4-yl]-pyrimidin-2-yl}-(3-methyl-benzyl)-amine in the form of an amorphous, colorless solid; MS: 499 (M+H)⁺;

15)—with 4-bromo-benzylamine, (4-bromo-benzyl)-{5-[(3RS,4RS)-3-(1,4-dimethoxy-naphthalen-2-ylmethoxy)-piperidin-4-yl]-pyrimidin-2-yl}-amine in the form of an amorphous, colorless solid; MS: 563, 565 (M+H)⁺.

The tert-butyl (3RS,4RS)-3-(1,4-dimethoxy-naphthalen-2-ylmethoxy)-4-(2-methylsulfonyl-pyrimidin-5-yl)-piperidine-1-carboxylate required as the starting material was prepared as follows:

(a) In an analogous manner to that described in Example 125 (g), from tert-butyl (3RS,4RS)-3-hydroxy-4-(2-methylsulfanyl-pyrimidin-5-yl)-piperidine-1-carboxylate [Example 129 (a)] and 2-chloromethyl-1,4-dimethoxy-naphthalene [J. Amer. Chem. Soc. 64, 2657 (1942)] there was obtained tert-butyl (3RS,4RS)-3-(1,4-dimethoxy-naphthalen-2-ylmethoxy)-4-(2-methylsulfanyl-pyrimidin-5-yl)-piperidine-1-carboxylate in the form of an amorphous, colorless solid; MS: 527 (M+H)⁺.

(b) In an analogous manner to that described in Example 129 (c), from tert-butyl (3RS,4RS)-3-(1,4-dimethoxy-naphthalen-2-yl-methoxy)-4-(2-methylsulfanyl-pyrimidin-5-yl)-piperidine-1-carboxylate by oxidation with m-chloroperbenzoic acid there was obtained tert-butyl (3RS,4RS)-3-(1,4-dimethoxy-naphthalen-2-ylmethoxy)-4-(2-methylsulfonyl-pyrimidin-5-yl)-piperidin-1-carboxylate in the form of a colorless solid; MS: 558 (M+H)⁺.

The 2-(5-phenyl-pyridin-2-yl)-ethanol and 2-(5-phenyl-pyridin-2-yl)-ethylamine used as condensation reagents were prepared as follows:

(α) 1.16 g (4 mmol) of 2-(2-benzyloxy-ethyl)-5-bromo-pyridine [Example 128 (b)], 139 mg (1.13 mmol) of tetrakis-(triphenylphosphine)-palladium and 537 mg (8.2 mmol) of phenylboric acid were dissolved in a small amout of ethanol and added all at once to 80 ml of toluene. Subsequently, 1.87 g (17.6 mmol) of sodium carbonate in 4.4 ml of water were added and the reaction mixture was heated under reflux for 7.5 hours under argon. After cooling the reaction solution was concentrated in a water-jet vacuum and partitioned between methylene chloride and water. The combined methylene chloride phases were dried over magnesium sulfate, filtered and concentrated in a water-jet vacuum. The thus obtained residue was thereupon chromatographed on silica gel with methylene chloride/ether (93:7). There were thus obtained 1.005 g (87% of theory) of 2-(2-benzyloxy-ethyl)-5-phenyl-pyridine as a colorless solid; R_{f}: 0.08 (SiO₂, methylene choride:ether=93:7).

(β) 1.0 g (3.5 mmol) of 2-(2-benzyloxy-ethyl)-5-phenyl-pyridine was dissolved in 1 ml of glacial acetic acid, treated with 1.8 ml (6.7 mmol) of hydrobromic acid in glacial acetic acid (30%) and the reaction mixture was stirred at room temperature for 18 hours. Thereupon, it was poured on to ice-water, extracted twice with hexane, the hexane phases were discarded and the aqueous phase was made alkaline with sodium carbonate solution and extracted three times with ether. The combined ether phases were dried over magnesium sulfate, filtered and concentrated in a water-jet vacuum. The thus obtained 2-(5-phenyl-pyridin-2-yl)-ethyl acetate was thereupon taken up in 12 ml of acetonitrile and treated with 5 ml of water and 4 ml of 2N sodium hydroxide solution and stirred at room temperature for 2.5 hours. Thereupon, aqueous ammonium chloride solution was added and the mixture was extracted with methylene chloride. The combined methylene chloride phases were dried over magnesium sulfate, filtered and concentrated in a water-jet vacuum. The thus obtained residue was thereupon chromatographed on silica gel with methylene chloride/ether (1:1). There was thus obtained 0.656 g (94% of theory) of 2-(5-phenyl-pyridin-2-yl)-ethanol as a colorless solid; R_{f}: 0.09 (SiO₂, methylene chloride:ether=1:1).

(γ) 0.473 g (2.4 mmol) of 2-(5-phenyl-pyridin-2-yl)-ethanol, 0.420 g (2.85 mmol) of phthalimide and 0.747 g (2.85 mmol) of triphenylphosphine were dissolved in 10 ml of tetrahydrofuran and the reaction mixture was thereupon treated under argon at −5° C. with 0.47 ml (3.0 mmol) of diethyl azodicarboxylate and stirred at −5° C. for a further 20 minutes and at room temperature for a further 18 hours. Thereupon, the reaction mixture was concentrated in a water-jet vacuum and the residue was chromatographed on silica gel with methylene chloride/methanol (98:2). There was thus obtained 0.657 g (83% of theory) of 2-[2-(5-phenyl-pyridin-2-yl)-ethyl]-isoindole-1,3-dione as a colorless solid; R.sub.f: 0.38 (SiO₂, hexane:ethyl acetate=1:1).

(δ) 0.657 g (2.0 mmol) of 2-[2-(5-phenyl-pyridin-2-yl)-ethyl]-isoindole-1,3-dione, 0.5 ml of hydrazine hydrate and 5 ml of ethanol were heated under reflux for 3.5 hours under argon. Thereupon, the mixture was diluted with 5 ml of ethanol and 20 ml of ether, filtered and the filtrate was concentrated in a water-jet vacuum. The thus obtained residue was subsequently chromatographed on silica gel with methylene chloride/methanol/conc. aq. ammonia (89:10:1). There was thus obtained 0.250 g (63% of theory) of 2-(5-phenyl-pyridin-2-yl)-ethylamine as a yellowish, amorphous solid; R_{f}: 0.17 (SiO₂, methylene chloride:methanol:conc. aq. ammonia=89:10:1).

### Example 140

The following compounds were obtained by reacting tert-butyl (3′RS,4′RS)-3′-(1,4-dimethoxy-naphthalen-2-ylmethoxy)-6-methylsulfonyl-3′,4′,5′,6′-tetrahydro-2′H-[3,4′]bipyridine-1′-carboxylate with alcohols in an analogous manner to that described in Example 129 (d) and subsequently cleaving off the BOC group by means of anhydrous zinc bromide in methylene chloride as described in Example 125 (h):

1)—With 3-cyclohexyl-propanol, (3′RS,4′RS)-6-(3-cyclohexyl-propoxy)-3′-(1,4-dimethoxy-naphthalen-2-ylmethoxy)-1′,2′,3′,4′,5′,6′-hexahydro-[3,4′]bipyridine in the form of a colorless solid; MS: 519 (M+H)⁺.

2)—With 3-(2-methoxy-benzyloxy)-propan-1-ol (prepared by alkylating propylene glycol in a large excess with 2-methoxy-benzyl chloride using sodium hydride in N,N-dimethylformamide), (3′RS,4′RS)-3′-(1,4-dimethoxy-naphthalen-2-ylmethoxy)-6-[3-(2-methoxybenzyloxy)-propoxy]-1′,2′,3′,4′,5′,6′-hexahydro-[3,4′]-bipyridine in the form of an amorphous, colorless solid; MS: 573 (M+H)⁺.

3)—With 4-cyclohexyl-butanol, (3′RS,4′RS)-6-(4-cyclohexyl-butoxy)-3′-(1,4-dimethoxy-naphthalen-2-ylmethoxy)-1′,2′,3′,4′,5′,6′-hexahydro-[3,4′]bipyridine in the form of an amorphous, colorless oil; MS: 533 (M+H)⁺.

The tert-butyl (3′RS,4′RS)-3′-(1,4-dimethoxy-naphthalen-2-ylmethoxy)-6-methylsulfonyl-3′,4′,5′,6′-tetrahydro-2′H-[3,4′]-bipyridine-1′-carboxylate used as the starting material was prepared as follows:

(a) In an analogous manner to that described in Example 127 (b)-(c), from tert-butyl 2-methylsulfanyl-5-bromo-pyridine [Tetrahedron 41, 1373 (1985)] via 2-methylsulfanyl-5-trimethylstannanyl-pyridine [colorless oil, MS: 289 (M)⁺], as well as 4-trifluormethylsulfonyloxy-3,6-dihydro-2H-pyridine-1-carboxylate there was obtained tert-butyl 6-methylsulfanyl-3′,6′-dihydro-2′H-[3,4′]bipyridine-1′-carboxylate in the form of a yellowish solid: MS: 307 (M+H)⁺.

(b) 1.5 g (4.9 mmol) of tert-butyl 6-methylsulfanyl-3′,6′-dihydro-2′H-[3,4′]bipyridine-1′-carboxylate were dissolved in 15 ml of 1,2-dimethoxyethane, treated at 3-4° C. with 8.8 ml of 1 molar borane-tetrahydrofuran solution and subsequently stirred at room temperature for 4 hours. Thereupon, while cooling with ice, 15 ml of water and subsequently portionwise 3.5 g (22.3 mmol) of solid sodium percarbonate were added and the reaction mixture was heated to 50° C. for 1 hour. Now, the reaction solution was partitioned between water and methylene chloride and the combined methylene chloride phases were washed with sodium pyrosulfite solution and water, dried over magnesium sulfate and concentrated. The thus obtained residue was subsequently chromatographed on silica gel with hexane/ethyl acetate (1:1). There were thus obtained 330 mg (21% of theory) of tert-butyl (3′RS,4′RS)-3′-hydroxy-6-methylsulfanyl-3′,4′,5′,6′-tetrahydro-2′H-[3,4′) bipyridine-1′-carboxylate in the form of a colorless amorphous solid; MS: 324 (M)⁺.

(c) In an analogous manner to that described in Example 125 (g), from tert-butyl (3′RS,4′RS)-3′-hydroxy-6-methylsulfanyl-3′,4′,5′,6′-tetrahydro-2′H-[3,4′]bipyridine-1′-carboxylate by alkylation with 2-chloromethyl-1,4-dimethoxy-naphthalene [J. Amer. Chem. Soc. 64, 2657 (1942)] there was obtained tert-butyl (3′RS,4′RS)-3′-(1,4-dimethoxy-naphthalen-2-ylmethoxy)-6-methylsulfanyl-3′,4′,5′,6′-tetrahydro-2′H-[3,4′]bipyridine-1′-carboxylate in the form of a colorless solid; MS: 526 (M+H)⁺.

(d) In an analogous manner to that described in Example 129 (c), from tert-butyl (3′RS,4′RS)-3′-(1,4-dimethoxy-naphthalen-2-ylmethoxy)-6-methylsulfanyl-3′,4′,5′,6′-tetrahydro-2′H-[3,4′]-bipyridine-1′-carboxylate by oxidation with m-chloroperbenzoic acid there was obtained tert-butyl (3′RS,4′RS)-3′-(1,4-dimethoxy-naphthalen-2-ylmethoxy)-6-methylsulfonyl-3′,4′,5′,6′-tetrahydro-2′H-[3,4′]bipyridine-1′-carboxylate in the form of an amorphous, colorless solid; MS: 557 (M+H)⁺.

### Example 141

(a) 50.0 g (0.6 mol) of 1,2,5,6-tetrahydropyridine and 135.3 g (0.6 mol) of di-tert-butyl dicarbonate in 1250 ml of water (deionized)/dioxan (3:2) were stirred at room temperature for 3 hours with the addition of 166.0 g (1.2 mol) of potassium carbonate (anhydrous). The mixture was poured on to ice-water, the product was extracted 3 times with 300 ml of ethyl acetate each time, the organic phases were washed twice with 500 ml of distilled water each time, then dried over magnesium sulfate, filtered and concentrated in a water-jet vacuum. The thus obtained crude product was chromatographed on silica gel with hexane and ethyl acetate. After drying in a high vacuum for 3 hours at room temperature there were thus obtained 109.4 g (99% of theory) of tert-butyl 3,6-dihydro-2H-pyridine-1-carboxylate as a pale yellow oil; MS: 183 (M)⁺.

(b) 127.0 g (0.6 mol) of m-chloroperbenzoic acid were dissolved in 1.5 l of methylene chloride under argon, then a solution of 108 g (0.59 mol) of tert-butyl 3,6-dihydro-2H-pyridine-1-carboxylate in 500 ml of methylene chloride was added dropwise at 5° C. within 1 hour and the mixture was subsequently stirred at room temperature overnight. The mixture was poured on to ice-water, adjusted to pH >8 with potassium carbonate solution and, after phase separation, back-extracted twice with 500 ml of methylene chloride each time; the organic phases were washed neutral twice with water, then dried over magnesium sulfate, filtered and the solvent was distilled off in a water-jet vacuum. The crude product was chromatographed on silica gel with hexane and ethyl acetate. There were thus obtained 86.64 g (74% of theory) of tert-butyl (1RS,6SR)-7-oxa-3-aza-bicyclo[4.1.0]heptane-3-carboxylate as a pale yellow oil; MS: 199 (M)⁺.

(c) 19.9 g (100 mmol) of tert-butyl (1RS,6SR)-7-oxa-3-aza-bicyclo[4.1.0]heptane-3-carboxylate and 32.5 g (500 mmol) of sodium azide were stirred at reflux for 3 hours with the addition of 39.1 g (250 mmol) of magnesium sulfate dihydrate in 500 ml of abs. methanol. The mixture was thereupon cooled to 10° C., filtered and the solvent was distilled off in a water-jet vacuum. The residue was taken up in 300 ml of methylene chloride, again filtered and the solvent was distilled off in a water-jet vacuum. The thus obtained crude product was chromatographed on silica gel with n-hexane and ethyl acetate. There were thus obtained 15.86 g (66% of theory) of tert-butyl (3RS,4RS)-4-azido-3-hydroxy-piperidine-1-carboxylate in the form of colorless crystals; MS: 242 (M)⁺.

(d) 15.4 g (63.5 mmol) of tert-butyl (3RS,4RS)-4-azido-3-hydroxy-piperidine-1-carboxylate and 15.5 g (69.9 mmol) of 2-bromomethyl-naphthalene were placed in 200 ml of dimethylformamide under argon at 5° C. Thereupon, 3.33 g (76.2 mmol) of sodium hydride dispersion (55% in mineral oil) were added in one portion with external cooling and then the mixture was stirred at room temperature overnight. The mixture was poured on to ice-water, the product was extracted 3 times with 200 ml of ethyl acetate each time, the organic phases were washed twice with 300 ml of water each time, then dried over magnesium sulfate, filtered and concentrated in a water-jet vacuum. The thus obtained crude product was chromatographed on silica gel with n-hexane and ethyl acetate. There were thus obtained 23.18 g (95% of theory) of tert-butyl (3RS,4RS)-4-azido-3-(naphthalen-2-ylmethoxy)-piperidine-1-carboxylate as a pale yellow oil; MS: 383 (M+H)⁺.

(e) 3.20 g (8.37 mmol) of tert-butyl (3RS,4RS)-4-azido-3-(naphthalen-2-ylmethoxy)-piperidine-1-carboxylate and 9.87 ml (176.3 mmol) of propargyl alcohol were stirred at reflux in 80 ml of toluene for 5 hours. After distilling off the solvent in a water-jet vacuum the crude product was chromatographed on silica gel with methylene chloride and methanol. There were thus obtained 1.47 g (40% of theory) of tert-butyl (3RS,4RS)-4-(4-hydroxymethyl-[1,2,3]triazol-1-yl)-3-(naphthalen-2-ylmethoxy)-4-piperidine-1-carboxylate in the form of colorless crystals [MS: 439 (M+H)⁺] and 0.81 g (22% of theory) of tert-butyl (3RS,4RS)-4-(5-hydroxymethyl-[1,2,3]triazol-1-yl)-3-(naphthalen-2-ylmethoxy)-4-piperidine-1-carboxylate in the form of colorless crystals; MS: 439 (M+H)⁺.

(f) 0.22 g (0.5 mmol) of tert-butyl (3RS,4RS)-4-(5-hydroxymethyl-[1,2,3]triazol-1-yl)-3-(naphthalen-2-ylmethoxy)-4-piperidine-1-carboxylate and 0.064 ml (0.5 mmol) of ortho-chloro-benzoyl chloride were placed in 10 ml of methylene chloride under argon at room temperature. Thereupon, there were added firstly while stirring 0.41 ml (3 mmol) of triethylamine and then 0.025 g (0.2 mmol) of 4-dimethylamino-pyridine and the mixture was stirred at room temperature for 18 hours. After distillation of the solvent in a water-jet vacuum the crude product was chromatographed on silica gel with methylene chloride and methanol. There were thus obtained 0.14 g (49% of theory) of tert-butyl (3RS,4RS)-4-[4-(2-chloro-benzoyloxymethyl)-[1,2,3]triazol-1-yl]-3-(naphthalen-2-ylmethoxy)-piperidine-1-carboxylate as a colorless resin; MS: 577 (M+H)⁺.

(g) In an analogous manner to that described in Example 136 (a), from tert-butyl (3RS,4RS)-4-[4-(2-chloro-benzoyloxymethyl)-[1,2,3]triazol-1-yl]-3-(naphthalen-2-ylmethoxy)-piperidine-1-carboxylate by cleavage of the BOC group with hydrogen chloride in methanol there was obtained 1-[(3RS,4RS)-3-(naphthalen-2-yl-methoxy)-piperidin-4-yl]-1H-[1,2,3]triazol-4-ylmethyl 2-chloro-benzoate hydrochloride (1:1) in the form of colorless crystals; MS: 477 (M+H)⁺.

### Example 142

The following compounds were obtained in an analogous manner to that described in Example 136 (a) by cleavage of the BOC group by means of hydrogen chloride in methanol:

1)—From tert-butyl (3RS,4RS)-4-(5-benzyloxymethyl-[1,2,3]triazol-1-yl)-3-(naphthalen-2-ylmethoxy)-piperidine-1-carboxylate, (3RS,4RS)-4-(5-benzyloxymethyl-[1,2,3]triazol-1-yl)-3-(naphthalen-2-ylmethoxy)-piperidine as a pale yellow oil; MS: 429 (M+H)⁺;

2)—from tert-butyl (3RS,4RS)-4-(4-benzyloxymethyl-[1,2,3]triazol-1-yl)-3-(naphthalen-2-ylmethoxy)-piperidine-1-carboxylate, (3RS,4RS)-4-(4-benzyloxymethyl-[1,2,3]triazol-1-yl)-3-(naphthalen-2-ylmethoxy)-piperidine hydrochloride (1:1) in the form of colorless crystals; MS: 429 (M+H)⁺;

3)—from tert-butyl (3RS,4RS)-4-[5-(3-benzyloxy-propoxy-methyl)-[1,2,3]triazol-1-yl]-3-(naphthalen-2-ylmethoxy)-piperidine-1-carboxylate, (3RS,4RS)-4-[5-(3-benzyloxy-propoxymethyl)-[1,2,3]triazol-1-yl]-3-(naphthalen-2-ylmethoxy)-piperidine as a colorless oil; MS: 487 (M+H)⁺;

4)—from tert-butyl (3RS,4RS)-4-[4-(3-benzyloxy-propoxy-methyl)-[1,2,3]triazol-1-yl]-3-(naphthalen-2-ylmethoxy)-piperidine-1-carboxylate, (3RS,4RS)-4-[4-(3-benzyloxy-propoxymethyl)-[1,2,3]triazol-1-yl]-3-(naphthalen-2-ylmethoxy)-piperidine as a colorless oil; MS: 487 (M+H)⁺.

The BOC derivatives used as starting materials were prepared as follows:

(a) 0.22 g (0.5 mmol) of tert-butyl (3RS,4RS)-4-(5-hydroxymethyl-[1,2,3]triazol-1-yl)-3-(naphthalen-2-ylmethoxy)-4-piperidine-1-carboxylate [Example 141 (e)] and 0.09 ml (0.75 mmol) of benzyl bromide were placed in 5 ml of dimethylformamide under argon at 5° C., then 0.044 g (1 mmol) of sodium hydride dispersion (55% in mineral oil) was added in one portion and the mixture was stirred at room temperature for 18 hours. The mixture was thereupon poured on to ice-water, the product was extracted 3 times with 30 ml of ethyl acetate each time and the organic phases were washed twice with 25 ml of distilled water each time, dried over magnesium sulfate, filtered and concentrated in a water-jet vacuum. The thus obtained crude product was chromatographed on silica gel with methylene chloride and methanol. There was thus obtained 0.24 g (91% of theory) of tert-butyl (3RS,4RS)-4-(5-benzyloxymethyl-[1,2,3]triazol-1-yl)-3-(naphthalen-2-ylmethoxy)-piperidine-1-carboxylate as a pale yellow oil; MS: 529 (M+H)⁺.

(b) In an analogous manner to that described in Example 142 (a), from tert-butyl (3RS,4RS)-4-(4-hydroxymethyl-[1,2,3]triazol-1-yl)-3-(naphthalen-2-ylmethoxy)-4-piperidine-1-carboxylate [Example 141 (e)] there was obtained tert-butyl (3RS,4RS)-4-(4-benzyloxymethyl-[1,2,3]triazol-1-yl)-3-(naphthalen-2-ylmethoxy)-piperidine-1-carboxylate as a yellow oil; MS: 529 (M+H)⁺.

(c) 0.22 g (0.5 mmol) of tert-butyl (3RS,4RS)-4-(5-hydroxymethyl-[1,2,3]triazol-1-yl)-3-(naphthalen-2-ylmethoxy)-4-piperidine-1-carboxylate [Example 141 (e)] and 0.13 ml (0.75 mmol) of 3-benzyloxy-propyl bromide were placed in 5 ml of dimethylformamide under argon at 5° C., then 0.17 g (1 mmol) of potassium iodide followed by 0.044 g (1 mmol) of sodium hydride dispersion (55% in mineral oil) were added all at once and thereafter the mixture was stirred at room temperature for 72 hours. The mixture was thereupon poured on to ice-water, the product was extracted 3 times with 30 ml of ethyl acetate each time, the organic phases were washed twice with 25 ml of distilled water each time, dried over magnesium sulfate, filtered and concentrated in a water-jet vacuum. The thus obtained crude product was chromatographed on silica gel with methylene chloride and methanol. There was thus obtained 0.27 g (92% of theory) of tert-butyl (3RS,4RS)-4-[5-(3-benzyloxy-propoxymethyl)-[1,2,3]triazol-1-yl]-3-(naphthalen-2-ylmethoxy)-piperidine-1-carboxylate as a pale yellow oil; MS: 586 (M)⁺.

(d) In an analogous manner to that described in Example 142 (c), from tert-butyl (3RS,4RS)-4-(4-hydroxymethyl-[1,2,3]triazol-1-yl)-3-(naphthalen-2-ylmethoxy)-4-piperidine-1-carboxylate Example 141 (e)] there was obtained tert-butyl (3RS,4RS)-4-[4-(3-benzyloxy-propoxymethyl)-[1,2,3]triazol-1-yl]-3-(naphthalen-2-ylmethoxy)-piperidine-1-carboxylate as a pale yellow oil; MS: 586 (M)⁺.

### Example 143

The following compounds were obtained in an analogous manner to that described in Example 136 (a) by cleavage of the BOC group by means of hydrogen chloride in methanol:

1)—From tert-butyl (3′RS,4′RS)-3′-(naphthalen-2-ylmethoxy)-3′,4′,5′,6′-tetrahydro-2′H-[2,4′]-bipyridine-1-carboxylate, (3′RS,4′RS)-3′-(naphthalen-2-ylmethoxy)-1′,2′,3′,4′,5′,6′-hexahydro-[2,4′]-bipyridine as a light yellow oil; MS: 319 (M+H)⁺;

2)—from tert-butyl (3′RS,4′RS)-3′-(naphthalen-2-ylmethoxy)-3′,4′,5′,6′-tetrahydro-2′H-[3,4′]-bipyridine-1-carboxylate, (3′RS,4′RS)-3′-(naphthalen-2-ylmethoxy)-1′,2′,3′,4′,5′,6′-hexahydro-[3,4′] bipyridine as a yellow oil; MS: 319 (M+H)⁺.

The BOC derivatives used as starting materials were prepared as follows:

(a) (α) A solution of 662 mg (2.0 mmol) of tert-butyl 4-trifluoromethyl-sulfonyloxy-3,6-dihydro-2H-pyridine-1-carboxylate [Example 126 (c)], 884 mg (2.4 mmol) of 2-tributylstannylpyridine (obtained from Maybridge Chemical Company) and 254 mg (6.0 mmol) of anhydrous lithium chloride in 30 ml of absolute DMF was flushed with argon, thereafter treated with 115 mg (0.1 mmol) of tetrakis-(triphenylphosphine)-palladium(IV) and then heated to reflux under argon for 3 hours. For the working-up, the reaction mixture was poured into 25 ml of 10% ammonia solution and finally stirred intensively for 5 minutes. The light yellow solution was treated with 100 ml of methylene chloride and stirred for 5 minutes. The organic phase was separated and the aqueous phase was extracted 3 times with 25 ml of methylene chloride each time. The combined organic phases were washed twice with 25 ml of water each time, dried over magnesium sulfate, and finally the solvent was distilled off under reduced pressure. The crude product was purified by flash chromatography on silica gel with a 2:1 mixture of hexane and ethyl acetate as the eluent. There were obtained 354 mg (68% of theory) of tert-butyl 3′,6′-dihydro-2′H-[2,4′]bipyridine-1′-carboxylate as a yellow oil; MS: 261 (M+H)⁺.

(β) A solution of 1.30 g (5.0 mmol) of tert-butyl 3′,6′-dihydro-2′H-[2,4′]bipyridine-1′-carboxylate in 15 ml of absolute tetrahydrofuran was treated dropwise at 0° C. under argon with 1.0 ml (801 mg, 10.0 mmol) of borane-dimethyl sulfide complex (95%) in dimethyl sulfide. The mixture was heated to boiling such that a slow distillation of the solvent took place (about 1 drop per minute). After 3 hours 3 ml of 2N sodium hydroxide solution and 2 ml of hydrogen peroxide solution (30%) were added dropwise at 0° C. The mixture was stirred at 50° C. for 6 hours, then cooled to room temperature and, for working-up, poured into a mixture of 200 ml of ether, 200 ml of water and 25 ml of sodium pyrosulfite solution (10%) while stirring vigorously. The organic phase was separated and the aqueous phase was extracted three times with 50 ml of ether each time. The combined organic phases were washed twice with 25 ml of water each time, dried over magnesium sulfate and finally the solvent was distilled off under reduced pressure. The crude product was purified by flash chromatography on silica gel with a 1:1 mixture of hexane and ethyl acetate as the eluent. There were obtained 354 mg (24% of theory) of tert-butyl (3′RS,4′RS)-3′-hydroxy-3′,4′,5′,6′-tetrahydro-2′H-[2,4′]bipyridine-1′-carboxylate as a yellow oil; MS: 279 (M+H)⁺.

(γ) In an analogous manner to that described in Example 125 (g), by alkylating tert-butyl (3′RS,4′RS)-3′-hydroxy-3′,4′,5′,6′-tetrahydro-2′H-[2,4′]bipyridine-1′-carboxylate with 2-bromomethynaphthalene there was obtained tert-butyl (3′RS,4′RS)-3′-(naphthalen-2-ylmethoxy)-3′,4′,5′,6′-tetrahydro-2′H-[2,4′]-bipyridine-1-carboxylate as a colorless resin; MS: 419 (M+H)⁺.

(b) (α) A solution of 16.7 g (106 mmol) of 3-bromo-pyridine in 200 ml of tert-butyl methyl ether was cooled to −75° C. Thereto there was added dropwise within 45 minutes a solution of 66 ml (106 mmol) of n-butyllithium (1.6M in hexane) and the mixture was stirred at −75° C. for one hour. Subsequently, a solution of 10.0 g (52.8 mmol) of 1-benzyl-4-piperidone in 50 ml of tert-butyl methyl ether was added dropwise at −70° C. to −75° C. and thereafter the mixture was stirred for 2 hours. Subsequently, the mixture was left to warm to room temperature. Thereafter, it was hydrolyzed with 50 ml of water and extracted with 100 ml of ethyl acetate. The organic phase was dried over magnesium sulfate and finally the solvent was evaporated under reduced pressure, with the product beginning to separate. From the evaporated mother liquor there were isolated by crystallization from a mixture of ethyl acetate and hexane a further 1.9 g, so that a total of 8.4 g (60% of theory) of 1′-benzyl-2′,3′,5′,6′-tetrahydro-1′H-[3,4′]bipyridinyl-4′-ol were obtained as a colorless solid; MS: 268 (M+H)⁺.

(β) A dispersion of 4.52 g (16.8 mmol) of 1′-benzyl-2′,3′,5′,6′-tetrahydro-1′H-[3,4′]bipyridinyl-4′-ol and 18 g (71 mmol) of potassium disulfate in 35 ml of decalin was stirred at 190° C. for 30 minutes. After cooling to room temperature the reaction mixure was dissolved in water and extracted twice with 50 ml of toluene each time. Subsequently, the aqueous phase was made alkaline with sodium hydroxide solution and extracted with ethyl acetate. The ethyl acetate phase was thereafter dried over sodium sulfate and evaporated under reduced pressure. For purification, the crude product was chromatographed on silica gel using a 98:2:0.1 mixture of methylene chloride, methanol and ammonia as the eluent. There were obtained 4.03 g (61% of theory) of 1′-benzyl-1′,2′,3′,6′-tetrahydro-3,4′-bipyridine as a yellowish oil; MS: 250 (M)⁺.

(γ) In an analogous manner to that described in Example 126 (f), by hydroboration of 1′-benzyl-1′,2′,3′,6′-tetrahydro-3,4′-bipyridine there was obtained (3′RS,4′RS)-1-benzyl-1′,2′,3′,4′,5′,6′-hexahydro-[3,4′]bipyridinyl-3′-ol as a yellowish oil; MS: 268 (M)⁺. Subsequent cleavage of the benzyl group by means of catalytic hydrogenation in the presence of palladium-charcoal (10%) at room temperature under normal pressure in methanol for 18 hours yielded (3′RS,4′RS)-1′,2′,3′,4′,5′,6′-hexahydro-[3,4′]bipyridinyl-3′-ol, which after reaction with di-tert-butyl-dicarbonate analogously to Example 141 (a) yielded tert-butyl(3′RS,4′RS)-3′-hydroxy-3′,4′,5′,6′-tetrahydro-2′H-[3,4′]bipyridine-1′-carboxylate as a yellowish oil; MS: 279 (M+H)⁺. Subsequent alkylation with 2-bromomethyl-naphthalene analogously to Example 125 (g) yielded tert-butyl (3′RS,4′RS)-3′-(naphthalen-2-ylmethoxy)-3′,4′,5′,6′-tetrahydro-2′H-[3,4′]-bipyridine-1-carboxylate, which was used as the crude product in the cleavage reaction of the BOC group by means of hydrogen chloride in methanol.

### Example 144

The following compounds were obtained by cleavage of the BOC group:

1)—From tert-butyl (3′RS,4′RS)-6-(3-benzyloxy-propoxy)-3′-(naphthalen-2-ylmethoxy)-3′,4′,5′,6′-tetrahydro-2′H-[3,4′]bipyridine-1′-carboxylate by means of zinc bromide in methylene chloride analogously to Example 125 (h), (3′RS,4′RS)-6-(3-benzyloxy-propoxy)-3′-(naphthalen-2-ylmethoxy)-1′,2′,3′,4′,5′,6′-hexahydro-[3,4′]bipyridine as a colorless solid; MS: 483 (M+H)⁺;

2)—from tert-butyl (3′RS,4′RS)-5-(2-benzyloxy-ethoxymethyl)-3′-(naphthalen-2-ylmethoxy)-3′,4′,5′,6′-tetrahydro-2′H-[2,4′]bipyridine-1′-carboxylate by means of hydrogen chloride in methanol analogously to Example 136 (a), (3′RS,4′RS)-5-(2-benzyloxy-ethoxymethyl)-3′-(naphthalen-2-ylmethoxy)-1′,2′,3′,4′,5′,6′-hexahydro-[2,4′]bipyridine as a light yellow oil; MS:483 (M+H)⁺.

The BOC derivatives used as starting materials were prepared as follows:

(a) (α) A solution of 10.62 g (40.2 mmol) of 2-benzyloxy-5-bromopyridine [J. Org. Chem. 60, 1408 (1995)] and 10.0 ml (15.8 g, 48.2 mmol) of hexamethyldistannate in 100 ml of absolute dioxane was flushed with argon and treated with 2.32 g (2.0 mmol) of tetrakis-(triphenylphoshine)-palladium(IV). The mixture was boiled under reflux for 15 hours. For the working-up, the dark solution was filtered over Speedex and the solvent was distilled off under reduced pressure. The residue was dissolved in 300 ml of methylene chloride, washed twice with 100 ml of water each time, dried over magnesium sulfate and finally the solvent was distilled off under reduced pressure. The crude product was purified by flash chromatography on silica gel with a 3:1 mixture of hexane and methylene chloride as the eluent. There were obtained 10.4 g (74% of theory) of 2-benzyloxy-5-trimethylstannyl-pyridine as a colorless oil; MS: 349 (M+H⁺).

(β) In an analogous manner to that described above, by a palladium catalyzed coupling of 2-benzyloxy-5-trimethylstannyl-pyridine with tert-butyl 4-trifluoromethylsulfonyloxy-3,6-dihydro-2H-pyridine-1-carboxylate [Example 126 (c)] there was obtained tert-butyl 6-benzyloxy-3′,6′-dihydro-2H-[3,4′]bipyridine-1′-carboxylate as a colorless solid MS: 367 (M+H)⁺.

(γ) A solution of 0.75 g (2.04 mmol) of tert-butyl 6-benzyloxy-3′,6′-dihydro-2H-[3,4]bipyridine-1′-carboxylate in 3 ml of absolute tetrahydrofuran was treated dropwise at 0° C. under argon with 0.42 ml (4.20 mmol) of borane-dimethyl sulfide complex (95%) in dimethyl sulfide. The mixture was heated to 60° C. and simultaneously a weak stream of argon was conducted through. After 1.5 hours 3 ml of tetrahydrofuran and 2 ml of water were added dropwise at 0° C. Subsequently, 740 mg (4.71 mmol) of sodium percarbonate were added portionwise, the mixture was warmed to room temperature and subsequently heated at 60° C. for 1 hour. Thereafter, the mixture was cooled to room temperature and, for working-up, was poured into a mixture of 100 ml of ether, 100 ml of water and 10 ml of 10% sodium pyrosulfite solution while stirring vigorously. The organic phase was separated and the aqueous phase was extracted 3 times with 25 ml of ether each time. The combined organic phases were washed twice with 10 ml of water each time, dried over magnesium sulfate and finally the solvent was distilled off under reduced pressure. The crude product (0.9 g) was purified by flash chromatography on silica gel with a 4:1 mixture of methylene chloride and ethyl acetate as the eluent. There was obtained 0.630 g (80% of theory) of a 4:1 mixture of tert-butyl (3′RS,4′RS)-6-benzyloxy-3′-hydroxy-3′,4′,5′,6′-tetrahydro-2′H-[3,4′]bipyridine-1′-carboxylate and tert-butyl 6-benzyloxy-4′-hydroxy-3′,4′,5′,6′-tetrahydro-2′H-[3,4′]bipyridine-1′-carboxylate as a colorless solid; MS: 385 (M+H)⁺.

(δ) A solution of 256 mg (0.67 mmol) of a 4:1 mixture of tert-butyl (3′RS,4′RS)-6-benzyloxy-3′-hydroxy-3′,4′,5′,6′-tetrahydro-2′H-(3,4′]bipyridine-1′-carboxylate and tert-butyl 6-benzyloxy-4′-hydroxy-3′,4′,5′,6′-tetrahydro-2′H-[3,4′]bipyridine-1′-carboxylate in 6 ml of absolute methanol was treated with 40 mg of palladium-charcoal (10%) and hydrogenated at normal pressure for 6 hours. After filtration of the catalyst and subsequent distillation of the solvent under reduced pressure there was obtained a 4:1 mixture of tert-butyl (3′RS,4′RS)-3′-hydroxy-6-oxo-1,6,3′,4′,5′,6′-hexahydro-2′H-[3,4′]bipyridine-1′-carboxylate and tert-butyl 4′-hydroxy-6-oxo-1,6,3′,4′,5′,6′-hexahydro-2′H-[3,4′]bipyridine-1′-carboxylate as a colorless solid; MS: 2-95 (M+H)⁺.

(ε) A solution of 74 mg (0.25 mmol) of a 4:1 mixture of tert-butyl (3′RS,4′RS)-3′-hydroxy-6-oxo-1,6,3′,4′,5′,6′-hexahydro-2′H-[3,4′]bipyridine-1′-carboxylate and 4′-hydroxy-6-oxo-1,6,3′,4′,5′,6′-hexahydro-2′H-[3,4′]bipyridine-1′-carboxylate, 99 mg (0.375 mmol) of triphenylphosphine and 52 mg (0.312 mmol) of 3-benzyloxy-1-propanol in 5 ml of absolute tetrahydrofuran was treated portionwise with 69 mg (0.30 mmol) of di-tert-butyl azodicarboxylate and stirred at room temperature for 6 hours. Subsequently, the reaction mixture was treate with 0.25 ml of methanol and, for working-up, was poured into 10 ml of methylene chloride and 10 ml of water while stirring vigorously. The organic phase was separated and the aqueous phase was extracted three times with 10 ml of methylene chloride each time. The combined organic phases were washed twice with 100 ml of water each time, dried over sodium sulfate and finally the solvent was distilled off under reduced pressure. The crude product (280 mg) was purified by flash chromatograpy on silica gel with a 4:1 mixture of methylene chloride and ethyl acetate as the eluent. There were obtained 37 mg (34% of theory) of a 4:1 mixture of tert-butyl (3′RS,4′RS)-6-(3-benzyloxy-propoxy)-3′-hydroxy-3′,4′,5′,6′-tetrahydro-2′H-[3,4′]bipyridine-1-carboxylate and tert-butyl 6-(3-benzyloxy-propoxy)-4′-hydroxy-3′,4′,5′,6′-tetrahydro-2′H-[3,4′]bipyridine-1′-carboxylate as a colorless oil; MS: 443 (M+H)⁺.

(ζ) In an analogous manner to that described in Example 125 (g), by alkylating a 4:1 mixture of tert-butyl (3′RS,4′RS)-6-(3-benzyloxy-propoxy)-3′-hydroxy-3′,4′,5′,6′-tetrahydro-2′H-[3,4′]bipyridine-1′-carboxylate and tert-butyl 6-(3-benzyloxy-propoxy)-4′-hydroxy-3′,4′,5′,6′-tetrahydro-2′H-[3,4′]bipyridine-1′-carboxylate with 2-bromomethylnaphthalene and subsequent separation of the isomers there was obtained tert-butyl (3′RS,4′RS)-6-(3-benzyloxy-propoxy)-3′-(naphthalen-2-ylmethoxy)-3′,4′,5′,6′-tetrahydro-2′H-[3,4′]bipyridine-1′-carboxylate as a colorless oil; MS: 583 (M+H)⁺.

(b) (α) Firstly, a solution of 53 ml (86 mmol) of methyllithium (1.6M in ether) was prepared at 0° C. in 100 ml of absolute tetrahydrofuran. Thereafter, a solution of 20 ml (96.8 mmol) of hexamethyldistannate in 100 ml of absolute tetrahydrofuran was added dropwise within 30 minutes at 0° C. and the mixture was stirred at 0° C. for 30 minutes. The pale yellow solution was cooled to −78° C. A solution of 14.2 g (71.3 mmol) of N-tert-butoxycarbonyl-4-piperidone in 80 ml of absolute tetrahydrofuran was added dropwise thereto at −78° C. within 45 minutes. After 4 hours at −78° C. 60 ml of saturated potassium sodium tartrate solution were added dropwise and the mixture was warmed to room temperature. The organic phase was separated and the aqueous phase was extracted three times with 200 ml of ether each time. The combined organic phases were washed twice with 100 ml of saturated ammonium chloride solution each time and twice with 100 ml of saturated sodium chloride solution each time, dried over magnesium sulfate and finally the solvent was distilled off under reduced pressure. The resulting yellow oil (23.1 g) was dissolved in 250 ml of methylene chloride, treated with 25.6 ml (183.9 mmol) of triethylamine and cooled to 0° C. A solution of 9.82 ml (18.4 mmol) of methanesulfonyl chloride in 90 ml of methylene chloride was added dropwise within 1 hour at 0° C. and the mixture was then stirred at 0° C. for 1 hour. Subsequently, 28.3 ml (190.3 mmol) of 1,8-diazabicyclo[5.4.0]undec-7-ene (DBU) were added dropwise at 0° C. within 30 minutes. The red solution was stirred at room temperature for 15 hours. For the working-up, the mixture was treated with 200 ml of water while stirring vigorously. The organic phase was separated and the aqueous phase was extracted three times with 100 ml of methylene chloride each time. The combined organic phases were washed twice with 100 ml of saturated sodium chloride solution each time, dried over magnesium sulfate and finally the solvent was distilled off under reduced pressure. The crude product (21.4 g) was purified by flash chromatography on silica gel with methylene chloride at the eluent. There were obtained 12.9 g (37.2 mmol, 52% of theory) of tert-butyl 4-trimethylstannyl-3,6-2H-pyridine-1-carboxylate as a yellow oil; MS: 348 (M+H)⁺.

(β) In an analogous manner to that described above, by a palladium catalyzed coupling of tert-butyl 4-trimethylstannyl-3,6-2H-pyridine-1-carboxylate with (rac)-2-chloro-5-[(tetrahydro-2H-pyran-2-yloxy)methyl]-pyridine [EP 475 273] there was obtained tert-butyl (RS)-5-(tetrahydro-pyran-2-yloxymethyl)-3′,6′-dihydro-2′H-[2,4′]bipyridine-1′-carboxylate as a yellow oil; MS: 375 (M+H)⁺.

(γ) A solution of 2.56 g (6.84 mmol) of tert-butyl (RS)-5-(Tetrahydro-pyran-2-yloxymethyl)-3′,6′-dihydro-2′H-[2,4′]bipyridine-1′-carboxylate in 10 ml of absolute tetrahydrofuran was treated dropwise at 0° C. under argon with 1.40 ml (1.12 g, 14.0 mmol) of borane-dimethyl sulfide complex (95%) in dimethyl sulfide. The mixture was heated to 50° C. and simultaneously a weak stream of argon was passed through. After 45 minutes 10 ml of tetrahydrofuran were added, the mixture was cooled to 0° C. and 1.58 g (21.0 mmol) of solid trimethylamine N-oxide were added portionwise, with the temperature being held at 5-10° C. The mixture was warmed to room temperature, heated under reflux for 1 hour, treated with 10 ml of methanol and subsequently heated under reflux for a further hour. The mixture was cooled to room temperature and, for working-up, poured into a mixture of 200 ml of methylene chloride, 200 ml of water and 10 ml of 2N sodium hydroxide solution while stirring vigorously. The organic phase was separated and the aqueous phase was extracted three times with 50 ml of methylene chloride each time. The combined organic phases were washed twice with 50 ml of sodium pyrosulfite solution (10%) (the pH being adjusted to about 9 by the addition of 2N sodium hydroxide solution) and twice with 25 ml of water, thereafter dried over magnesium sulfate and finally evaporated under reduced pressure. The crude product (2.59 g) was purified by flash chromatography on silica gel with a 1:1 mixture of methylene chloride and ethyl acetate as the eluent. There was obtained 0.280 g (10% of theory) of a 1:1 mixture of tert-butyl (3′RS,4′RS)-3′-hydroxy-5-[(RS)- and -[(SR)-tetrahydro-pyran-2-yloxymethyl]-3′,4′,5′,6′-tetrahydro-2′H-[2,4′]bipyridine-1′-carboxylate as a light yellow oil; MS: 393 (M+H)⁺.

(δ) In an analogous manner to that described in Example 125 (g), by alkylating a 1:1 mixture of tert-butyl (3′RS,4′RS)-3′-hydroxy-5-[(RS)- and -[(SR)-tetrahydro-pyran-2-yloxymethyl]-3′,4′,5′,6′-tetrahydro-2′H-[2,4′]bipyridine-1′-carboxylate with 2-bromomethylnaphthalene there was obtained a 1:1 mixture of tert-butyl (3′RS,4′RS)-3′-(naphthalen-2-ylmethoxy)-5-[(RS)- and -[(SR)-tetrahydro-pyran-2-yloxymethyl]-3′,4′,5′,6′-tetrahydro-2′H-[2,4′]bipyridine-1-carboxylate as a yellow oil; MS: 533 (M+H)⁺.

(ε) A solution of 102 mg (0.19 mmol) of a 1:1 mixture of tert-butyl (3′RS,4′RS)-3′-(naphthalen-2-ylmethoxy)-5-[(RS)- and -[(SR)-tetrahydro-pyran-2-yloxymethyl]-3′,4′,5′,6′-tetrahydro-2′H-[2,4′]bipyridine-1-carboxylate was dissolved in 2 ml of methanol and cooled to −15° C. 2 ml of a 2N solution of hydrogen chloride in methanol were added dropwise within 2 minutes at −10 to −15° C. The mixture was warmed to room temperature and stirred for 30 minutes, and then, for working-up, partitioned between 25 ml of ethyl acetate and 25 ml of aqueous 5% sodium hydrogen carbonate solution. The organic phase was separated and the aqueous phase was extracted three times with 10 ml of ethyl acetate each time. The combined ethyl acetate phases were dried over magnesium sulfate and finally evaporated under reduced pressure. The crude product (96 mg) was purified by chromatography on silica gel with a 1:1 mixture of ethyl acetate and methylene chloride as the eluent. There were obtained 78 mg (92% of theory) of tert-butyl (3′RS,4′RS)-5-hydroxymethyl-3′-(naphthalen-2-ylmethoxy)-3′,4′,5′,6′-tetrahydro-2′H-[2,4′]bipyridine-1′-carboxylate as a light yellow oil; MS: 449 (M+H)⁺.

(ζ) A solution of 70 mg (0.156 mmol) of tert-butyl (3′RS,4′RS)-5-hydroxymethyl-3′-(naphthalen-2-ylmethoxy)-3′,4′,5′,6-tetrahydro-2′H-[2,4′]bipyridine-1′-carboxylate in 2 ml of absolute DMF was cooled to −10° C. and treated with 26 .mu.1 (19 mg, 0.187 mmol) of triethylamine. 13 .mu.1 (20 mg, 0.172 mmol) of methanesulfonyl chloride and 2 mg of N,N-dimethylaminopyridine (DMAP) were added at −10 to −15° C. and subsequently the mixture was stirred at 0° C. for 1 hour. For the working-up, the mixture was partitioned between 25 ml of ethyl acetate and 25 ml of aqueous 5% ammonium chloride solution and the organic phase was separated. The aqueous phase was extracted three times with 10 ml of ethyl acetate each time. The combined ethyl acetate phases were dried over magnesium sulfate and finally evaporated under reduced pressure. The crude product was purified by chromatography on silica gel with a 1:1 mixture of ethyl acetate and hexane as the eluent. There were obtained 22 mg (35% of theory) of tert-butyl (3′RS,4′RS)-5-chloromethyl-3′-(naphthalen-2-ylmethoxy)-3′,4′,5′,6′-tetrahydro-2′H-[2,4′]bipyridine-1′-carboxylate as a light yellow oil; MS: 467, 469 (M+H)⁺.

(η) A solution of 22 mg (0.047 mmol) of tert-butyl (3′RS,4′RS)-5-chloromethyl-3′-(naphthalen-2-ylmethoxy)-3′,4′,5′,6′-tetrahydro-2′H-[2,4′]bipyridine-1′-carboxylate in 0.5 ml of DMF was treated with 67 μl (72 mg 0.47 mmol) of 2-benzyloxyethanol and 19 mg (0.47 mmol) of sodium hydride (60% dispersion in oil) and subsequently stirred at room temperature for 2 hours. For the working-up, the mixture was partitioned between 15 ml of ethyl acetate and 15 ml of aqueous 5% ammonium chloride solution and then the organic phase was separated. The aqueous phase was extracted three times with 5 ml of ethyl acetate each time. The combined ethyl acetate phases were dried over magnesium sulfate and finally the solvent was distilled off under reduced pressure. The crude tert-butyl (3′RS,4′RS)-5-(2-benzyloxy-ethoxymethyl)-3′-(naphthalen-2-ylmethoxy)-3′,4′,5′,6′-tetrahydro-2′H-[2,4′]bipyridine-1′-carboxylate was used in the next step without further purification and characterization.

### Example 145

(a) A solution of 10.8 g (54.3 mmol) of tert-butyl (1RS,6SR)-7-oxa-3-aza-bicyclo[4.1.0]heptane-3-carboxylate [Example 141 (b)] in 250 ml of acetonitrile was treated with 7.98 g (162.9 mmol) of powdered sodium cyanide and 17.3 g (162.0 mmol) of lithium perchlorate and the reaction mixture was stirred at 95° C. under argon for 24 hours. For the working-up, the brownish solution was cooled, treated with 150 ml of ethyl acetate and filtered over Decalite. The filtrate was washed with 100 ml of water and the phases were separated. The aqueous phase was adjusted to pH 5 and extracted three times with 60 ml of ethyl acetate each time. The combined organic phases were dried over sodium sulfate and evaporated under reduced pressure. For purification, the residue was chromatographed on silica gel using a 4:1 mixture of hexane and ethyl acetate as the eluent. There were obtained 9.9 g (80.5% of theory) of a 4:1 mixture of tert-butyl (3RS,4RS)-4-cyano-3-hydroxy-piperidine-1-carboxylate and tert-butyl (3RS,4SR)-3-cyano-4-hydroxy-piperidine-1-carboxylate in the form of colorless crystals; MS: 227 (M+H)⁺.

(b) A solution of 5.8 g (25.6 mmol) of a 4:1 mixture of tert-butyl (3RS,4RS)-4-cyano-3-hydroxy-piperidine-1-carboxylate and tert-butyl (3RS,4SR)-3-cyano-4-hydroxy-piperidine-1-carboxylate in 50 ml of N,N-dimethylformamide was stirred at room temperature under argon for 4 hours with 1.8 g (38.4 mmol) of sodium hydrogen sulfide monohydrate and 2.05 g (38.4 mmol) of ammonium chloride. For the working-up, the reaction mixture was evaporated under reduced pressure and the residue was taken up in 150 ml of methylene chloride. The solution obtained was washed twice with 10 ml of water each time and the organic phase was separated, dried over sodium sulfate and then evaporated under reduced pressure. For purification and separation of the isomer mixture, the residue was chromatographed on silica gel using a 95:5:0.1 mixture of methylene chloride, methanol and ammonia as the eluent. There were obtained 3.48 g (52% of theory) of tert-butyl (3RS,4SR)-3-hydroxy-4-thiocarbamoyl-piperidine-1-carboxylate, (R_{f}: 0.37, silica gel; methylene chloride:methanol:ammonia=90:10:0.1 v/v/v), MS: 260 (M)⁺, as well as 1.2 g of a mixture of tert-butyl (3RS,4SR)-3-hydroxy-4-thiocarbamoyl-piperidine-1-carboxylate and tert-butyl (3RS,4RS)-4-hydroxy-3-thiocarbamoyl-piperidine-1-carboxylate, each as a colorless oil.

(c) 4 ml of methyl iodide were added at room temperature to a solution of 1.88 g (7.22 mmol) of tert-butyl (3RS,4SR)-3-hydroxy-4-thiocarbamoyl-piperidine-1-carboxylate in 5 ml of acetone. After stirring at room temperature for 14 hours the product had separated. 10 ml of ether were added and the reaction mixture was stirred for 30 minutes. After filtration and drying there were obtained 2.76 g (98% of theory) of (3RS,4SR)-[amino-(]-tert-butoxycarbonyl-3-hydroxy-piperidine-4-yl)-methylene-methyl-sulfonium iodide as colorless crystals; R_{f}: 0.22 (silica gel; methylene chloride:methanol:ammonia=95:5:0.1 v/v/v).

(d) A solution of 2.76 g (7.09 mmol) of (3RS,4SR)-[amino-(1-tert-butoxycarbonyl-3-hydroxy-piperidine-4-yl)-methylene-methyl-sulfonium iodide in 15 ml of methanol was treated with 0.41 g (3.55 mmol) of ammonium carbonate and stirred at room temperature for 18 hours. For the working-up, the reaction mixture was evaporated under reduced pressure. 2.5 g (95% of theory) of tert-butyl (3RS,4RS)-4-carbamimidoyl-3-hydroxy-piperidine-1-carboxylate iodide were obtained as a colorless foam. A solution of 715 mg (1.92 mmol) of tert-butyl (3RS,4RS)-4-carbamimidoyl-3-hydroxy-piperidine-1-carboxylate iodide in 20 ml of methanol was stirred at room temperature for 1 hours with 321 mg (1.92 mmol) of silver acetate. The precipitated silver iodide was filtered off and washed with 20 ml of methanol. The light yellow filtrate obtained was evaporated under reduced pressure. There were obtained 538 mg (92% of theory) of tert-butyl (3RS,4RS)-4-carbamimidoyl-3-hydroxy-piperidine-1-carboxylate acetate as a colorless foam.

(e) A solution of 372 mg (1 mmol) of tert-butyl (3RS,4RS)-4-carbamimidoyl-3-hydroxy-piperidine-1-carboxylate acetate in 10 ml of methanol was treated with 1 ml of 1N sodium methylate solution and stirred at room temperature. Subsequently, 205 mg (1 mmol) of 2-benzyloxy-3-dimethylamino-acrolein (EPA 0 477 901) were added thereto and the solution was heated to reflux for 18 hours. For the working-up, the reaction mixture was evaporated under reduced pressure and the residue was taken up in 20 ml of methylene chloride and washed with 5 ml of water. The organic phase was separated, dried over sodium sulfate and finally evaporated under reduced pressure. For purification, the residue was chromatographed on silica gel using a 1:4 mixture of methylene chloride and ethyl acetate as the eluent. There were obtained 106 mg (27% of theory) of tert-butyl (3RS,4RS)-4-(5-benzyloxy-pyrimidin-2-yl)-3-hydroxy-piperidine-1-carboxylate as a colorless oil; MS: 386 (M+H)⁺.

(f) A solution of 110 mg (0.29 mmol) of tert-butyl (3RS,4RS)-4-(5-benzyloxy-pyrimidin-2-yl)-3-hydroxy-piperidine-1-carboxylate in 5 ml of methanol was treated with 20 mg of 5% palladium-charcoal and hydrogenated at room temperature for 12 hours. For the working-up, the catalyst was filtered off and washed with 20 ml of methanol. The methanol solution was evaporated under reduced pressure and the resulting oil was crystallized by the addition of ether. There were obtained 80 mg (93% of theory) of tert-butyl (3RS,4RS)-3-hydroxy-4-(5-hydroxy-pyrimidin-2-yl)-piperidine-1-carboxylate in the form of colorless, slightly delequesent crystals; MS: 296 (M+H)⁺.

(g) A mixture of 65 mg (0.22 mmol) of tert-butyl (3RS,4RS)-3-hydroxy-4-(5-hydroxy-pyrimidin-2-yl)-piperidine-1-carboxylate and 151.2 mg (0.66 mmol) of (3-bromo-propoxymethyl)-benzene in 10 ml of methyl ethyl ketone was stirred at 80° C. for 48 hours under argon. For the working-up, the reaction mixture was evaporated under reduced pressure, the residue was taken up in methylene chloride and the solution was chromatographed directly on silica gel using a 1:4 mixture of methylene chloride and ethyl acetate as the eluent. There were obtained 40 mg (41% of theory) of tert-butyl (3RS,4RS)-4-[5-(3-benzyloxy-propoxy)-pyrimidin-2-yl]-3-hydroxy-piperidine-1-carboxylate as a foam; R_{f}: 0.43 (silica gel; methylene chloride:ethyl acetate=1:4 v/v).

(h) A solution of 40 mg (0.09 mmol) of tert-butyl (3RS,4RS)-4-[5-(3-benzyloxy-propoxy)-pyrimidin-2-yl]-3-hydroxy-piperidine-1-carboxylate and 23 mg (0.1 mmol) of 2-bromomethyl-naphthalene in 5 ml of N,N-dimethylformamide was treated with 5 mg (0.1 mmol) of sodium hydride (50% dispersion in oil) and stirred at room temperature for 18 hours. For the working-up, the reaction mixture was evaporated under reduced pressure, the residue obtained was taken up in 3 ml of methylene chloride and the solution was chromatographed directly on silica gel using a 2:1 mixture of hexane and ethyl acetate as the eluent. There were obtained 40 mg (76% of theory) of tert-butyl (3RS,4RS)-4-[5-(3-benzyloxy-propoxy)-pyrimidin-2-yl]-3-(naphthalen-2-ylmethoxy)-piperidine-1-carboxylate as an oil; MS: 584 (M+H)⁺.

(i) A solution of 40 mg (0.07 mmol) of tert-butyl (3RS,4RS)-4-[5-(3-benzyloxy-propoxy)-pyrimidin-2-yl]-3-(naphthalen-2-ylmethoxy)-piperidine-1-carboxylate and 0.1 ml of trifluoroacetic acid in 1 ml of methylene chloride was stirred at room temperature for 2 hours. Thereafter, the solution was evaporated and the residue was taken up in 1 ml of ethyl acetate and crystallized by the addition of hexane. There were obtained 25 mg (60% of theory) of (3RS,4RS)-5-(3-benzyloxy-propoxy)-2-[3-(naphthalen-2-ylmethoxy)-piperidin-4-yl]-pyrimidine trifluoroacetate in the form of colorless crystals; MS: 484 (M+H)⁺.

### Example 146

(a) In an analogous manner to that described in Example 145 (a), by epoxide opening by means of 4-benzyloxy-2(1H)-pyridone [Chem. Pharm. Bull. 22, 763-770 (1974)], from tert-butyl (1RS,6SR)-7-oxa-3-aza-bicyclo[4.1.0]heptane-3-carboxylate [Example 141 (b)] there was obtained a 1:1 mixture of tert-butyl (3′RS,4′RS)-4-benzyloxy-4′-hydroxy-2-oxo-3′,4′,5′,6′-tetrahydro-2H-2′H-[1,3′]bipyridine-1′-carboxylate and tert-butyl (3′RS,4′RS)-4-benzyloxy-3′-hydroxy-2-oxo-3′,4′,5′,6′-tetrahydro-2H-2′H-[1,4′]bipyridine-1′-carboxylate as colorless crystals; MS: 401 (M+H)⁺.

(b) In an analogous manner to that described in Example 145 (f), from a 1:1 mixture of tert-butyl (3′RS,4′RS)-4-benzyloxy-4′-hydroxy-2-oxo-3′,4′,5′,6′-tetrahydro-2H-2′H-[1,3′]bipyridine-1′-carboxylate and tert-butyl (3′RS,4′RS)-4-benzyloxy-3′-hydroxy-2-oxo-3′,4′,5′,6′-tetrahydro-2H-2′H-[1,4′]bipyridine-1′-carboxylate by means of catalytic hydrogenation there was obtained a 1:1 mixture of tert-butyl (3′RS,4′RS)-4,3′-dihydroxy-2-oxo-3′,4′,5′,6′-tetrahydro-2H,2′H-[1,4′]bipyridine-1′-carboxylate and tert-butyl (3′RS,4′RS)-4,4′-dihydroxy-2-oxo-3′,4′,5′,6′-tetrahydro-2H,2′H-[1,3′]bipyridine-1′-carboxylate as a colorless foam; MS: 311 (M+H)⁺.

(c) In an analogous manner to that described in Example 145 (g), from a 1:1 mixture of tert-butyl (3′RS,4′RS)-4,3′-dihydroxy-2-oxo-3′,4′,5′,6′-tetrahydro-2H,2′H-[1,4′]bipyridine-1′-carboxylate and tert-butyl (3′RS,4′RS)-4,4′-dihydroxy-2-oxo-3′,4′,5′,6′-tetrahydro-2H,2′H-[1,3′]bipyridine-1′-carboxylate by reaction with (3-bromo-propoxymethyl)-benzene in the presence of potassium carbonate there was obtained a mixture of tert-butyl (3′RS,4′RS)-4-(3-benzyloxy-propoxy)-3′-hydroxy-2-oxo-3′,4′,5′,6′-tetrahydro-2H,2′H-[1,4′]bipyridine-1′-carboxylate and tert-butyl (3′RS,4′RS)-4-(3-benzyloxy-propoxy)-4′-hydroxy-2-oxo-3′,4′,5′,6′-tetrahydro-2H,2′H-[1,3′]bipyridine-1′-carboxylate as a colorless oil; MS: 459 (M+H)⁺.

(d) In an analogous manner to that described in Example 145 (h), from a mixture of tert-butyl (3′RS,4′RS)-4-(3-benzyloxy-propoxy)-3′-hydroxy-2-oxo-3′,4′,5′,6′-tetrahydro-2H,2′H-[1,4′]bipyridine-1′-carboxylate and tert-butyl (3′RS,4′RS)-4-(3-benzyloxy-propoxy)-4′-hydroxy-2-oxo-3′,4′,5′,6′-tetrahydro-2H,2′H-[1,3′]bipyridine-1′-carboxylate by means of alkylation with 2-bromomethyl-naphthalene and after chromatographic separation of the two isomers on silica gel using a 1:4 mixture of methylene chloride and ethyl acetate as the eluent there was obtained tert-butyl (3′RS,4′RS)-4-(3-benzyloxy-propoxy)-4′-(naphthalen-2-ylmethoxy)-2-oxo-3′,4′,5′,6′-tetrahydro-2H,2′H-[1,3′]bipyridine-1′-carboxylate, (R_{f}: 0.64, SiO₂; methylene chloride:ethyl acetate=1:4 v/v), and tert-butyl (3′RS,4′RS)-4-(3-benzyloxy-propoxy)-3′-(naphthalen-2-ylmethoxy)-2-oxo-3′,4′,5′,6′-tetrahydro-2H,2′H-[1,4′]bipyridine-1′-carboxylate, MS: 599 (M+H)⁺, (R_{f}: 0.44, SiO₂; methylene chloride:ethyl acetate=1:4 v/v), each as a yellowish oil.

(e) In an analogous manner to that described in Example 145 (i), from tert-butyl (3′RS,4′RS)-4-(3-benzyloxy-propoxy)-3′-(naphthalen-2-ylmethoxy)-2-oxo-3′,4′,5′,6′-tetrahydro-2H,2′H-[1,4′]bipyridine-1′-carboxylate by cleavage of the BOC group by means of trifluoroacetic acid there was obtained (3′RS,4′RS)-4-(3-benzyloxy-propoxy)-3′-(naphthalen-2-ylmethoxy)-1′,2′,3′,4′,5′,6′-hexahydro-[1,4′]bipyridin-2-one trifluoroacetate as a colorless solid; MS: 499 (M+H)⁺.

### Example 147

The following compounds were obtained in an analogous manner to that described in Example 22 (l) by cleavage of the BOC group by means of hydrogen chloride in methanol:

1)—From tert-butyl (1RS,2RS,3RS,5SR)-3-[4-(2-benzyloxy-ethoxymethyl)-phenyl]-2-(naphthalen-2-ylmethoxy)-8-aza-bicyclo[3.2.1]octane-8-carboxylate, (1RS,2RS,3RS,5SR)-3-[4-(2-benzyloxy-ethoxymethyl)-phenyl]-2-(naphthalen-2-yl-methoxy)-8-aza-bicyclo[3.2.1]octane as a colorless oil; R_{f}: 0.15 (silica gel; methylene chloride:methanol:ammonia=95:5:0.1);

2)—from tert-butyl (1RS,2RS,3RS,5SR)-2-(naphthalen-2-ylmethoxy)-3-[4-(2-phenoxy-ethoxymethyl)-phenyl]-8-aza-bicyclo[3.2.1]octane-8-carboxylate, (1RS,2RS,3 RS,5SR)-2-(naphthalen-2-ylmethoxy)-3-[4-(2-phenoxy-ethoxymethyl)-phenyl]-8-aza-bicyclo[3.2.1]octane as a yellow oil; R_{f}: 0.21 (silica gel; methylene chloride:methanol:ammonia=95:5:0.1);

3)—from tert-butyl (1RS,2RS,3RS,5SR)-3-(4-benzyloxymethyl-phenyl)-2-(naphthalen-2-ylmethoxy)-8-aza-bicyclo3.2.1]octane-8-carboxylate, (1RS,2RS,3RS,5SR)-3-(4-benzyloxymethyl-phenyl)-2-(naphthalen-2-ylmethoxy)-8-aza-bicyclo[3.2.1]octane hydrochloride as a colorless oil; MS: 464 (M+H)⁺;

4)—from tert-butyl (1RS,2RS,3RS,5SR)-2-(naphthalen-2-ylmethoxy)-3-(4-phenylsulfanylmethyl-phenyl)-8-aza-bicyclo-[3.2.1]octane-8-carboxylate, (1RS,2RS,3RS,5SR)-2-(naphthalen-2-ylmethoxy)-3-(4-phenylsulfanylmethyl-phenyl)-8-aza-bicyclo[3.2.1]octane hydrochloride as a colorless oil; MS: 566 (M+H)⁺;

5)—from tert-butyl (1RS,2RS,3RS,5SR)-3-[4-(2-chloro-benzoyloxymethyl)-phenyl]-2-(naphthalen-2-ylmethoxy)-8-aza-bicyclo[3.2.1]octane-8-carboxylate, (1RS,2RS,3RS,5SR)-4-[2-(naphthalen-2-ylmethoxy)-8-aza-bicyclo[3.2.1]oct-3-yl]-benzyl 2-chloro-benzoate as a yellowish foam; MS: 512 (M+H)⁺;

The BOC compounds used as starting materials were prepared as follows:

(a) In an analogous manner to that described in Example 1 (g), by alkylating tert-butyl (1RS,2RS,3RS,5SR)-3-(4-hydroxymethyl-phenyl)-2-(naphthalen-2-ylmethoxy)-8-aza-bicyclo[3.2.1]octane-8-carboxylate [Example 86 (eee)] with 2-(benzyloxy)-ethyl iodide [Helv. Chim. Acta Vol. 71, (1988), 2039] there was obtained tert-butyl (1RS,2RS,3RS,5SR)-3-[4-(2-benzyloxy-ethoxymethyl)-phenyl]-2-(naphthalen-2-ylmethoxy)-8-aza-bicyclo[3.2.1]octane-8-carboxylate as a colorless oil; MS: 625 (M+NH₄)⁺.

(b) In an analogous manner to that described in Example 1 (g), by alkylating tert-butyl (1RS,2RS,3RS,5SR)-3-(4-hydroxymethyl-phenyl)-2-(naphthalen-2-ylmethoxy)-8-aza-bicyclo[3.2.1]octane-8-carboxylate [Example 86 (eee)] with phenoxy-ethyl bromide there was obtained tert-butyl (1RS,2RS,3RS,5SR)-2-(naphthalen-2-ylmethoxy)-3-[4-(2-phenoxy-ethoxymethyl)-phenyl]-8-aza-bicyclo[3.2.1]octane-8-carboxylate as a colorless oil; MS: 611 (M+NH₄)⁺.

(c) In an analogous manner to that described in Example 1 (g), by alkylating tert-butyl (1RS,2RS,3RS,5SR)-3-(4-hydroxymethyl-phenyl)-2-(naphthalen-2-ylmethoxy)-8-aza-bicyclo[3.2.1]octane-8-carboxylate [Example 86 (eee)] with benzyl bromide there was obtained tert-butyl (1RS,2RS,3RS,5SR)-3-(4-benzyloxymethyl-phenyl)-2-(naphthalen-2-ylmethoxy)-8-aza-bicyclo[3.2.1]octane-8-carboxylate, which was used as the crude product in the reaction for BOC cleavage.

(d) In an analogous manner to that described in Example 33 (a), by reacting tert-butyl (1RS,2RS,3RS,5SR)-3-(4-hydroxymethyl-phenyl)-2-(naphthalen-2-ylmethoxy)-8-aza-bicyclo[3.2.1]octane-8-carboxylate [Example 86 (eee)] with diphenyl disulfide in the presence of tributylphosphine there was obtained tert-butyl (1RS,2RS,3RS,5SR)-2-(naphthalen-2-ylmethoxy)-3-(4-phenyl-sulfanylmethyl-phenyl)-8-aza-bicyclo[3.2.1]octane-8-carboxylate as a colorless oil; MS: 566 (M+H)⁺.

(e) In an analogous manner to that described in Example 22 (k), by esterifying tert-butyl (1RS,2RS,3RS,5SR)-3-(4-hydroxymethyl-phenyl)-2-(naphthalen-2-ylmethoxy)-8-aza-bicyclo[3.2.1]octane-8-carboxylate [Example-86 (eee)] with 2-chloro-benzoyl chloride there was obtained tert-butyl (1RS,2RS,3RS,5SR)-4-[2-(naphthalen-2-ylmethoxy)-8-aza-bicyclo[3.2.1]oct-3-yl]-benzyl 2-chloro-benzoate as a colorless oil, which was used as the crude product in the reaction for BOC cleavage.

### Example 148

In an analogous manner to that described in Example 22 (l), from tert-butyl (3SR,4RS,5RS)-4-[4-(3-benzyloxy-propoxy)-phenyl]-3-methoxymethyl-5-(naphthalen-2-ylmethoxy)-piperidine-1-carboxylate by cleavage of the BOC group by means of hydrogen chloride in methanol there was obtained (3SR,4RS,5RS)-4-[4-(3-benzyloxy-propoxy)-phenyl]-3-methoxymethyl-5-(naphthalen-2-ylmethoxy)-piperidine as an amorphous, colorless solid; MS: 526 (M+H)⁺.

The tert-butyl (3SR,4RS,5RS)-4-[4-(3-benzyloxy-propoxy)-phenyl]-3-methoxymethyl-5-(naphthalen-2-ylmethoxy)-piperidine-1-carboxylate used as the starting material was synthesized as follows:

(a) In an analogous manner to that described in Example 68 (a)-(b), firstly the primary hydroxy function of (3RS,4RS)- and (3SR,4RS)-1-benzyl-3-hydroxymethyl-piperidin-4-ol [E. Jaeger and J. H. Biel, J. Org. Chem. 30 (3), 740-744 (1965)] was protected by using triphenylchloromethane analogously to Example 22 (h) in pyridine in place of tert-butyldiphenylchlorosilane and there was thus obtained (3RS,4RS)- and (3SR,4RS)-1-benzyl-3-trityloxymethyl-piperidin-4-ol. Subsequent oxidation with oxalyl chloride in dimethyl sulfoxide yielded (RS)-1-benzyl-3-trityloxymethyl-piperidin-4-one as a colorless foam; MS: 462 (M+H)⁺. Subsequent reaction with 4-iodoanisole analogously to Example 62 (b) yielded a mixture of (3RS,4RS)- and (3RS,4SR)-1-benzyl-4-(4-methoxy-phenyl)-3-trityloxymethyl-piperidin-4-ol as a colorless solid; MS: 570 (M+H)⁺.

(b) A solution of 5.5 ml (58.7 mmol) of phosphorus oxychloride in 20 ml of dry pyridine was added dropwise within 20 minutes to a solution of 8.36 g (14.6 mmol) of a mixture of (3RS,4RS)- and (3RS,4SR)-1-benzyl-4-(4-methoxy-phenyl)-3-trityloxymethyl-piperidin-4-ol in 20 ml of dry pyridine. The reaction mixture was stirred at 60° C. for 20 hours. The dark red reaction mixture was cooled and evaporated under reduced pressure. The residue was taken up in methylene chloride and treated with saturated sodium carbonate solution. The organic phase was separated, dried over sodium sulfate and evaporated under reduced pressure. The residue was chromatographed on silica gel using a 20:1 mixture of toluene and ethyl acetate as the eluent. There were obtained 6.1 g (75% of theory) of (RS)-1-benzyl-4-(4-methoxy-phenyl)-3-trityloxymethyl-1,2,3,6-tetrahydro-pyridine as a colorless solid; MS: 552 (M+H)⁺.

(c) In an analogous manner to that described in Example 62 (d), by hydroboration of (RS)-1-benzyl-4-(4-methoxy-phenyl)-3-trityloxymethyl-1,2,3,6-tetrahydro-pyridine with borane-tetrahydrofuran and subsequent oxidation by means of sodium percarbonate there was obtained (3RS,4RS,5SR)-1-benzyl-4-(4-methoxy-phenyl)-5-trityloxymethyl-piperidin-3-ol as a colorless foam; MS: 570 (M+H)⁺.

(d) In an analogous manner to that described in Example 44 (d), by simultaneous cleavage of the methoxy and trityloxy groups by means of boron tribromide in methylene chloride from tert-butyl (3RS,4RS,5SR)-1-benzyl-4-(4-methoxy-phenyl)-5-trityloxy-methyl-piperidin-3-ol there was obtained (3RS,4RS,5 SR)-1-benzyl-5-hydroxymethyl-4-(4-hydroxy-phenyl)-piperidin-3-ol hydrobromide as a white solid; MS: 314 (M+H)⁺.

(e) In an analogous manner to that described in Example 2 (e), by catalytic hydrogenation at atmospheric pressure using a 10% palladium-charcoal catalyst in methanol from tert-butyl (3RS,4RS,5SR)-1-benzyl-5-hydroxymethyl-4-(4-hydroxy-phenyl)-piperidin-3-ol hydrobromide there was obtained (3RS,4RS,5SR)-5-hydroxymethyl-4-(4-hydroxy-phenyl)-piperidin-3-ol hydrobromide as a colorless foam; MS: 224 (M+H)⁺.

(f) In an analogous manner to that described in Example 1 (f), by reacting (3RS,4RS,5SR)-5-hydroxymethyl-4-(4-hydroxy-phenyl)-piperidin-3-ol hydrobromide with di-tert-butyl dicarbonate there was obtained tert-butyl (3RS,4RS,5SR)-3-hydroxy-5-hydroxy-methyl-4-(4-hydroxy-phenyl)-piperidine-1-carboxylate as a colorless foam.

(g) In an analogous manner to that described in Example 44 (e), by alkylating tert-butyl (3RS,4RS,5SR)-3-hydroxy-5-hydroxymethyl-4-(4-hydroxy-phenyl)-piperidine-1-carboxylate with benzyl 3-bromopropyl ether in the presence of potassium carbonate in butan-2-one there was obtained tert-butyl (3RS,4RS,5SR)-4-[4-(3-benzyloxy-propoxy)-phenyl]-3-hydroxy-5-hydroxymethyl-piperidine-1-carboxylate as a colorless oil; MS: 472 (M+H)⁺.

(h) In an analogous manner to that described in Example 22 (h), by reacting tert-butyl (3RS,4RS,5SR)-4-[4-(3-benzyloxy-propoxy)-phenyl]-3-hydroxy-5-hydroxymethyl-piperidine-1-carboxylate with triphenylchloromethane in pyridine there was obtained tert-butyl (3RS,4RS,5SR)-4-[4-(3-benzyloxy-propoxy)-phenyl]-3-hydroxy-5-trityloxymethyl-piperidine-1-carboxylate, [MS: 731 (M+NH₄)⁺], as a colorless foam, alkylation of which with 2-bromomethyl-naphthalene analogously to Example 62 (h) gave tert-butyl (3RS,4RS,5SR)-4-[4-(3-benzyloxy-propoxy)-phenyl]-3-(naphthalen-2-ylmethoxy)-5-trityloxymethyl-piperidine-1-carboxylate, [MS: 871 (M+NH₄)⁺], as a colorless foam. Subsequent selective cleavage of the trityl group by means of a mixture of trifluoroacetic acid and trifluoroacetic anhydride in methylene chloride analogously to Example 86 (u) yielded tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate as a colorless solid; MS: 612 (M+H)⁺. Subsequent alkylation with methyl iodide analogously to Example 62 (h) gave tert-butyl (3SR,4RS,5RS)-4-[4-(3-benzyloxy-propoxy)-phenyl]-3-methoxymethyl-5-(naphthalen-2-ylmethoxy)-piperidine-1-carboxylate as a colorless solid; MS: 626 (M+H)⁺.

### Example 149

The following compounds were obtained in an analogous manner to that described in Example 22 (l) by cleavage of the BOC group by means of hydrogen chloride in methanol:

1)—From tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)], (3SR,4RS,5RS)-[4-[4-(3-benzyloxy-propoxy)-phenyl]-5-(naphthalen-2-ylmethoxy)-piperidin-3-yl]-methanol hydrochloride as a colorless solid; MS: 512 (M+H)⁺;

2)—from tert-butyl (3RS,4RS,5SR)-4-[4-(3-benzyloxy-propoxy)-phenyl]-3-(naphthalen-2-ylmethoxy)-5-(pyridine-4-ylsulfanylmethyl)-piperidine-1-carboxylate, (3SR,4RS,5RS)-4-[4-[4-(3-benzyloxy-propoxy)-phenyl]-5-(naphthalen-2-ylmethoxy)-piperidin-3-ylmethylsulfanyl]-pyridine hydrochloride as a colorless solid; MS: 605 (M+H)⁺;

3)—from tert-butyl (3RS,4RS,5SR)-4-[4-(3-benzyloxy-propoxy)-phenyl]-3-(naphthalen-2-ylmethoxy)-5-phenylsulfanylmethyl-piperidine-1-carboxylate, (3RS,4RS,5SR)-4-[4-(3-benzyloxy-propoxy)-phenyl]-3-(naphthalen-2-ylmethoxy)-5-phenylsulfanylmethyl-piperidine hydrochloride as a colorless oil; MS: 604 (M+H)⁺;

4)—from tert-butyl (3SR,4RS,5RS)-4-[4-(3-benzyloxy-propoxy)-phenyl]-3-(2-morpholin-4-yl-ethoxymethyl)-5-(naphthalen-2-ylmethoxy)-piperidine-1-carboxylate, (3SR,4RS,5RS)-4-[2-[4-[4-(3-benzyloxy-propoxy)-phenyl]-5-(naphthalen-2-ylmethoxy)-piperidin-3-ylmethoxy)-ethyl]-morpholine as a colorless oil; MS: 625 (M+H)⁺;

5)—from tert-butyl (3RS,4RS,5RS)-4-[4-(3-benzyloxy-propoxy)-phenyl]-3-diethylaminomethyl-5-(naphthalen-2-ylmethoxy)-piperidine-1-carboxylate, (3SR,4RS,5RS)-[4-[4-(3-benzyloxy-propoxy)-phenyl]-5-(naphthalen-2-ylmethoxy)-piperidin-3-ylmethyl]-diethyl-amine as a yellowish oil; MS: 567 (M+H)⁺;

6)—from tert-butyl (3RS,4RS,5RS)-4-[4-(3-benzyloxy-propoxy)-phenyl]-3-[[(2-dimethylamino-ethyl)-methyl-amino]-methyl]-5-(naphthalen-2-ylmethoxy)-piperidine-1-carboxylate, (3SR,4RS,5RS)-N-[4-[4-(3-benzyloxy-propoxy)-phenyl]-5-(naphthalen-2-ylmethoxy)-piperidin-3-ylmethyl]-N,N′,N′-trimethyl-ethane-1,2-diamine as a yellowish oil; MS: 596 (M+H)⁺;

7)—from tert-butyl (3RS,4RS,5SR)-4-[4-(3-benzyloxy-propoxy)-phenyl]-3-(naphthalen-2-ylmethoxy)-5-[1,2,4]triazol-1-ylmethyl-piperidine-1-carboxylate, (3RS,4RS,5SR)-4-[4-(3-benzyloxy-propoxy)-phenyl]-3-(naphthalen-2-ylmethoxy)-5-[1,2,4]triazol-1-ylmethyl-piperidine hydrochloride as a colorless solid; MS: 563 (M+H)⁺;

8)—from tert-butyl (3RS,4RS,5RS)-4-[4-(3-benzyloxy-propoxy)-phenyl]-3-(naphthalen-2-ylmethoxy)-5-(2-oxo-imidazolidin-1-ylmethyl)-piperidine-1-carboxylate, (3SR,4RS,5RS)-1-[4-[4-(3-benzyloxy-propoxy)-phenyl]-5-(naphthalen-2-ylmethoxy)-piperidin-3-ylmethyl]-imidazolidin-2-one s a colorless solid; MS: 580 (M+H)⁺;

9) from tert-butyl (3SR,4RS,5RS)-4-[4-(3-benzyloxy-propoxy)-phenyl]-3-(naphthalen-2-ylmethoxy)-5-sulfooxymethyl-piperidine-1-carboxylate trimethylammonium salt, mono-(3SR,4RS,5RS)-[4-[4-(3-benzyloxy-propoxy)-phenyl]-5-(naphthalen-2-ylmethoxy)-piperidin-3-ylmethyl]sulfate as a colorless solid; MS: 590 (M-H)⁻.

The BOC derivatives used as starting materials were prepared as follows:

(a) In an analogous manner to that described in Example 33 (a), by reacting tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)] with 4,4′-dithiopyridine in the presence of tributylphosphine there was obtained tert-butyl (3RS,4RS,5SR)-4-[4-(3-benzyloxy-propoxy)-phenyl]-3-(naphthalen-2-ylmethoxy)-5-(pyridine-4-ylsulfanylmethyl)-piperidine-1-carboxylate as a yellow, semi-solid substance; MS: 705 (M+H)⁺.

(b) In an analogous manner to that described in Example 33 (a), by reacting tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)] with diphenyl disulfide in the presence of tributylphosphine there was obtained tert-butyl (3RS,4RS,5SR)-4-[4-(3-benzyloxy-propoxy)-phenyl]-3-(naphthalen-2-ylmethoxy)-5-phenylsulfanylmethyl-piperidine-1-carboxylate as a colorless oil; MS: 721 (M+NH₄)⁺.

(c) In an analogous manner to that described in Example 1 (g), by alkylating tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)] with 4-(2-chloroethyl)-morpholine there was obtained tert-butyl (3SR,4RS,5RS)-4-[4-(3-benzyloxy-propoxy)-phenyl]-3-(2-morpholin-4-yl-ethoxymethyl)-5-(naphthalen-2-ylmethoxy)-piperidine-1-carboxylate as a colorless oil; MS: 725 (M+H)⁺.

(d) By reacting tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)] with mesyl chloride according to a method known from the literature there was obtained tert-butyl (3SR,4RS,5RS)-4-[4-(3-benzyloxy-propoxy)-phenyl]-3-methanesulfonyloxymethyl-5-(naphthalen-2-ylmethoxy)-piperidine-1-carboxylate [MS: 707 (M+NH₄)⁺] as a colorless solid. Further reaction with diethylamine in acetonitrile at 50° C. analogously to Example 34 gave tert-butyl (3RS,4RS,5RS)-4-[4-(3-benzyloxy-propoxy)-phenyl]-3-diethylaminomethyl-5-(naphthalen-2-ylmethoxy)-piperidine-1-carboxylate as a brown foam; MS: 667 (M+H)⁺.

(e) Reaction of tert-butyl (3SR,4RS,5RS)-4-[4-(3-benzyloxy-propoxy)-phenyl]-3-methanesulfonyloxymethyl-5-(naphthalen-2-ylmethoxy)-piperidine-1-carboxylate with N,N,N′-trimethyl-ethylenediamine in dimethylformamide at 100° C. analogously to Example 34 yielded tert-butyl (3RS,4RS,5RS)-4-[4-(3-benzyloxy-propoxy)-phenyl]-3-[[(2-dimethylamino-ethyl)-methyl-amino]-methyl]-5-(naphthalen-2-ylmethoxy)-piperidine-1-carboxylate as a colorless oil; MS: 696 (M+H)⁺.

(f) A solution of 22 mg (0.32 mmol) of 1,2,4-triazole in 5 ml of DMF was cooled to 0° C. and treated with 15 mg (0.29 mmol) of sodium hydride (50% dispersion in refined oil). Subsequently, the mixture was left to warm to room temperature and was stirred for a further 1 hour. 70 mg (0.105 mmol) of tert-butyl (3SR,4RS,5RS)-4-[4-(3-benzyloxy-propoxy)-phenyl]-3-methanesulfonyloxymethyl-5-(naphthalen-2-yl methoxy)-piperidine-1-carboxylate were added to this solution. The reaction mixture was heated to 100° C. for 24 hours. For the working-up, the reaction mixture was evaporated under reduced pressure, the residue was partitioned between water and methylene chloride and the organic phase was separated, dried over sodium sulfate and evaporated. For purification, the crude product (70 mg) was chromatographed on silica gel using a 98:2 mixture of methylene-chloride and methanol as the eluent. There were obtained 52 mg (77% of theory) of tert-butyl (3RS,4RS,5SR)-4-[4-(3-benzyloxy-propoxy)-phenyl]-3-(naphthalen-2-ylmethoxy)-5-[1,2,4]triazol-1-ylmethyl-piperidine-1-carboxylate as a colorless solid; MS: 663 (M+H)⁺.

(g) In an analogous manner to that described in Example 149 (f), by reacting tert-butyl (3SR,4RS,5RS)-4-[4-(3-benzyloxy-propoxy)-phenyl]-3-methanesulfonyloxymethyl-5-(naphthalen-2-ylmethoxy)-piperidine-1-carboxylate with imidazolidin-2-one there was obtained tert-butyl (3RS,4RS,5RS)-4-[4-(3-benzyloxy-propoxy)-phenyl]-3-(naphthalen-2-ylmethoxy)-5-(2-oxo-imidazolidin-1-ylmethyl)-piperidine-1-carboxylate as a yellowish oil.

(h) A solution of 100 mg (0.136 mmol) of tert-butyl (3SR,4RS,5RS)-4-[4-(3-benzyloxy-propoxy)-pheny]]-3-hydroxymethyl-5-(naphthalen-2-ylmethoxy)-piperidine-1-carboxylate [Example 148 (h)] in 10 ml of dry pyridine was treated with 78 mg (0.43 mmol) of sulfur trioxide-trimethylamine complex and stirred at room temperature for 36 hours. For the working-up, the reaction mixture was evaporated under reduced pressure and the crude product was purified directly by flash chromatography using a 9:1 mixture of methylene chloride and methanol as the eluent. There were obtained 102 mg of tert-butyl (3SR,4RS,5RS)-4-[4-(3-benzyloxy-propoxy)-phenyl]-3-(naphthalen-2-ylmethoxy)-5-sulfooxymethyl-piperidine-1-carboxylate trimethylammonium salt as a colorless solid; MS: 690 (M-H)⁻.

### Example 150

The following compounds were obtained In an analogous manner to that described in Example 10 (b) by cleavage of the BOC group by means of zinc bromide in methylene chloride:

1)—From tert-butyl (3SR,4RS,5RS)-4-[4-(3-benzyloxy-propoxy)-phenyl]-3-[3-(4-methyl-piperazin-1-yl)-propyl-carbamoyloxymethyl]-5-(naphthalen-2-ylmethoxy)-piperidine-1-carboxylate, (3SR,4RS,5RS)-4-[4-(3-benzyloxy-propoxy)-phenyl]-5-(naphthalen-2-ylmethoxy)-piperidin-3-ylmethyl[3-(4-methyl-piperazin-1-yl)-propyl]-carbamate as a colorless solid; MS: 695 (M+H)⁺.

2)—From tert-butyl (3SR,4RS,5RS)-4-[4-(3-benzyloxy-propoxy)-phenyl]-3-(2-morpholin-4-yl-ethyl carbamoyloxymethyl)-5-(naphthalen-2-ylmethoxy)-piperidine-1-carboxylate, (3SR,4RS,5RS)-4-[4-(3-benzyloxypropoxy)-phenyl]-5-(naphthalen-2-ylmethoxy)-piperidin-3-ylmethyl (2-morpholin-4-yl-ethyl)-carbamate as a colorless oil; MS: 668 (M+H)⁺.

3)—From tert-butyl (3SR,4RS,5RS)-4-[4-(3-benzyloxy-propoxy)-phenyl]-3-carbamoyloxymethyl-5-(naphthalen-2-ylmethoxy)-piperidine-1-carboxylate, (3SR,4RS,5RS)-4-[4-(3-benzyloxy-propoxy)-phenyl]-5-(naphthalen-2-ylmethoxy)-piperidin-3-ylmethyl carbamate as a colorless solid; MS: 555 (M+H)⁺.

The BOC derivatives used as starting materials were prepared as follows:

(a) A mixture of 90 mg (0.15 mmol) of tert-butyl 3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)] and 60 mg (0.75 mmol) of lithium carbonate in 10 ml of dry tetrahydrofuran was cooled to 0° C. A solution of 0.90 ml (1.6 mmol) of phosgene in toluene (1.93N) was added dropwise thereto while cooling with ice. The mixture was stirred at room temperature overnight in order complete the reaction. The reaction mixture was evaporated under reduced pressure in order to remove the excess phosgene and the crude chloroformate obtained was taken up in 10 ml of tetrahydrofuran. This mixture was treated with 58 mg (0.38 mmol) of 1-(3-aminopropyl)-4-methylpiperizine and stirred at room temperature for 3 hours. For the working-up, the reaction mixture was diluted with 40 ml of methylene chloride and then extracted with 20 ml of water. The organic phase was dried over sodium sulfate and subsequently evaporated under reduced pressure. For purification, the residue was chromatographed on silica gel using a 95:5:0.1 mixture of methylene chloride, methanol and ammonia as the eluent. There were obtained 85 mg (73% of theory) of tert-butyl (3SR,4RS,5RS)-4-[4-(3-benzyloxy-propoxy)-phenyl]-3-[3-(4-methyl-piperazin-1-yl)-propylcarbamoyloxymethyl]-5-(naphthalen-2-ylmethoxy)-piperidine 1-carboxylate as a colorless solid; MS: 795 (M+H)⁺.

(b) In an analogous manner to that described in Example 150 (a), from tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)] there was synthesized the corresponding chloroformate, reaction of which with 4-(2-aminoethyl)-morpholine gave tert-butyl (3SR,4RS,5RS)-4-[4-(3-benzyloxy-propoxy)-phenyl]-3-(2-morpholin-4-yl-ethyl carbamoyl-oxymethyl)-5-(naphthalen-2-ylmethoxy)-piperidine-1-carboxylate as a colorless solid; MS: 768 (M+H)⁺.

(c) In an analogous manner to that described in Example 150 (a), from tert-butyl (3SR,4RS,5RS)-4-[4-(3-benzyloxy-propoxy)-phenyl]-3-hydroxymethyl-5-(naphthalen-2-ylmethoxy)-piperidine-1-carboxylate [Example 148 (h)] there was synthesized the corresponding chloroformate, reaction of which with a solution of ammonia in tetrahydrofuran gave tert-butyl (3SR,4RS,5RS)-4-[4-(3-benzyloxy-propoxy)-phenyl]-3-carbamoyloxymethyl-5-(naphthalen-2-ylmethoxy)-piperidine-1-carboxylate as a colorless solid; MS: 672 (M+NH₄)⁺.

### Example 151

The following compounds were obtained in an analogous manner to that described in Example 22 (l) by cleavage of the BOC group by means of hydrogen chloride in methanol:

1)—From tert-butyl (3RS,4RS)-4-[4-(2-benzyloxy-ethoxy-methyl)-phenyl]-3-(naphthalen-2-ylmethoxy)-piperidine-1-carboxylate, (3RS,4RS)-4-[4-(2-benzyloxy-ethoxymethyl)-phenyl]-3-(naphthalen-2-ylmethoxy)-piperidine as a colorless foam; MS: 482 (M+H)⁺;

2)—from tert-butyl (3SR,4RS,5RS)-4-[4-(2-benzyloxy-ethoxymethyl)-phenyl]-3-hydroxymethyl-5-(naphthalen-2-ylmethoxy)-piperidine-1-carboxylate, (3SR,4RS,5RS)-[4-[4-(2-benzyloxy-ethoxymethyl)-phenyl]-5-(naphthalen-2-ylmethoxy)-piperidin-3-yl]-methanol as a colorless oil; MS: 512 (M+H)⁺;

3)—from tert-butyl (3SR,4RS,5RS)-4-[4-(2-benzyloxy-ethoxymethyl)-phenyl]-3-methoxymethyl-5-(naphthalen-2-ylmethoxy)-piperidine-1-carboxylate, (3SR,4RS,5RS)-4-[4-(2-benzyloxy-ethoxymethyl)-phenyl]-3-methoxymethyl-5-(naphthalen-2-ylmethoxy)-piperidine as a colorless oil; MS: 526 (M+H)⁺;

4)—from tert-butyl (3RS,4RS,5SR)-4-[4-(2-benzyloxy-ethoxymethyl)-phenyl]-3-(naphthalen-2-ylmethoxy)-5-(pyridin-3-ylmethoxymethyl)-piperidine-1-carboxylate, (3SR,4RS,5RS)-3-[4-[4-(2-benzyloxy-ethoxymethyl)-phenyl]-5-(naphthalen-2-ylmethoxy)-piperidin-3-ylmethoxymethyl]-pyridine as a colorless oil; MS: 603 (M+H)⁺;

5)—from tert-butyl (3SR,4RS,5RS)-4-[4-(2-benzyloxy-ethoxymethyl)-phenyl]-3-(2-methoxy-ethoxymethyl)-5-(naphthalen-2-ylmethoxy)-piperidine-1-carboxylate, (3SR,4RS,5RS)-4-[4-(2-benzyloxy-ethoxymethyl)-phenyl]-3-(2-methoxy-ethoxymethyl)-5-(naphthalen-2-ylmethoxy)-piperidine as a colorless oil; MS: 570 (M+H)⁺;

6)—from a mixture of tert-butyl (3RS,4RS,5SR)- and (3SR,4SR,5RS)-4-[4-(2-benzyloxy-ethoxymethyl)-phenyl]-3-(naphthalen-2-ylmethoxy)-5-[3-[(RS)-tetrahydro-pyran-2-yloxy]-propoxymethyl]-piperidine-1-carboxylate with simultaneous cleavage of the THP group, (3SR,4RS,5RS)-3-[4-[4-(2-benzyloxy-ethoxymethyl)-phenyl]-5-(naphthalen-2-ylmethoxy)-piperidin-3-5ylmethoxy]-propan-1-ol as a colorless oil; MS: 570 (M+H)⁺;

7)—from tert-butyl (3SR,4RS,5RS)-3-methoxymethyl-4-{4-[(methyl-phenyl-carbamoyloxy)-methyl]-phenyl}-5-(naphthalen-2-ylmethoxy)-piperidine-1-carboxylate, (3SR,4RS,5RS)-4-[3-methoxymethyl-5-(naphthalen-2-ylmethoxy)-piperidin-4-yl]benzylmethyl-phenyl-carbamate as a colorless oil; MS: 525 (M+H)⁺;

8)—from tert-butyl (3SR,4RS,5RS)-4-[4-[(benzyl-methyl-carbamoyloxy)-methyl]-phenyl]-3-methoxy methyl-5-(naphthalen-2-ylmethoxy)-piperidine-1-carboxylate, (3SR,4RS,5RS)-4-[3-methoxymethyl-5-(naphthalen-2-ylmethoxy)-piperidin-4-yl]-benzyl benzyl-methyl-carbamate as a colorless oil; MS: 539 (M+H)⁺.

The BOC derivatives used as starting materials were obtained as follows:

(a) In an analogous manner to that described in Example 1 (g), by alkylating tert-butyl (3RS,4RS)-4-(4-hydroxymethyl-phenyl)-3-(naphthalen-2-ylmethoxy)-piperidine-1-carboxylate [Example 22 (j)] with 2-(benzyloxy)-ethyl iodide [Helv. Chim. Acta Vol. 71, (1988), 2039] there was obtained tert-butyl (3RS,4RS)-4-[4-(2-benzyloxy-ethoxymethyl)-phenyl]-3-(naphthalen-2-ylmethoxy)-piperidine-1-carboxylate as a colorless foam; MS: 599 (M+NH₄)⁺.

(b) In an analogous manner to that described in Example 22 (d), from tert-butyl (3RS,4RS,5SR)-1-benzyl-4-(4-bromo-phenyl)-5-hydroxymethyl-piperidin-3-ol (Example 68 (e)] by a palladium catalyzed carbonylation with carbon monoxide in methanol there was obtained methyl (3RS,4RS,5SR)-4-(1-benzyl-3-hydroxy-5-hydroxymethyl-piperidin-4-yl)-benzoate, hydrogenolysis of which in the presence of 5% palladium-charcoal at atmospheric pressure in methanol analogously to Example 2 (e) gave methyl 4-(3-hydroxy-5-hydroxymethyl-piperidin-4-yl)-benzoate. Subsequent introduction of the BOC group analogously to Example 1 (f) yielded tert-butyl (3RS,4RS,5SR)-3-hydroxy-5-hydroxymethyl-4-(4-methoxycarbonyl-phenyl)-piperidine-1-carboxylate, from which by reaction with triphenylchloromethane analogously to Example 68 (i) there was obtained tert-butyl (3RS,4RS,5SR)-3-hydroxy-4-(4-methoxycarbonyl-phenyl)-5-trityloxymethyl-piperidine-1-carboxylate. Subsequent alkylation by means of 2-bromomethyl-naphthalene analogously to Example 1 (g) yielded tert-butyl (3RS,4RS,5SR)-4-(4-methoxycarbonyl-phenyl)-3-(naphthalen-2-ylmethoxy)-5-trityloxymethyl-piperidine-1-carboxylate, reduction of which with lithium borohydride analogously to Example 22 (e) yielded tert-butyl (3RS,4RS,5SR)-4-(4-hydroxymethyl-phenyl)-3-(naphthalen-2-ylmethoxy)-5-trityloxymethyl-piperidine-1-carboxylate as a colorless foam; MS: 737 (M+NH₄)⁺. Further alkylation with 2-(benzyloxy)-ethyl iodide [Helv. Chim. Acta Vol. 71, (1988), 2039] analogously to Example 1 (g) gave tert-butyl (3RS,4RS,5SR)-4-[4-(2-benzyloxy-ethoxymethyl)-phenyl]-3-(naphthalen-2-ylmethoxy)-5-trityloxymethyl-piperidine-1-carboxylate, from which after cleavage of the trityl group by means of a mixture of trifluoroacetic acid and trifluoroacetic anhydride in methylene chloride analogously to Example 86 (u) there was obtained tert-butyl (3SR,4RS,5RS)-4-[4-(2-benzyloxy-ethoxymethyl)-phenyl]-3-hydroxymethyl-5-(naphthalen-2-ylmethoxy)-piperidine-1-carboxylate as a colorless oil; MS: 629 (M+NH₄)⁺.

(c) In an analogous manner to that described in Example 1 (g), by alkylating tert-butyl (3SR,4RS,5RS)-4-[4-(2-benzyloxy-ethoxymethyl)-phenyl]-3-hydroxymethyl-5-(naphthalen-2-ylmethoxy)-piperidine-1-carboxylate with methyl iodide there was obtained tert-butyl (3SR,4RS,5RS)-4-[4-(2-benzyloxy-ethoxymethyl)-phenyl]-3-methoxymethyl-5-(naphthalen-2-ylmethoxy)-piperidine-1-carboxylate as a colorless oil; MS: 643 (M+NH₄)⁺.

(d) In an analogous manner to that described in Example 1 (g), by alkylating tert-butyl (3SR,4RS,5RS)-4-[4-(2-benzyloxy-ethoxymethyl)-phenyl]-3-hydroxymethyl-5-(naphthalen-2-ylmethoxy)-piperidine-1-carboxylate with 3-(chloromethyl)-pyridine there was obtained tert-butyl (3RS,4RS,5SR)-4-[4-(2-benzyloxy-ethoxymethyl)-phenyl]-3-(naphthalen-2-ylmethoxy)-5-(pyridine-3-ylmethoxymethyl)-piperidine-1-carboxylate as a colorless oil; MS: 703 (M+H)⁺.

(e) In an analogous manner to that described in Example 1 (g), by alkylating tert-butyl (3SR,4RS,5RS)-4-[4-(2-benzyloxy-ethoxymethyl)-phenyl]-3-hydroxymethyl-5-(naphthalen-2-ylmethoxy)-piperidine-1-carboxylate with 2-methoxy-ethyl bromide there was obtained tert-butyl (3SR,4RS,5RS)-4-[4-(2-benzyloxy-ethoxymethyl)-phenyl]-3-(2-methoxy-ethoxymethyl)-5-(naphthalen-2-ylmethoxy)-piperidine-1-carboxylate as a colorless oil; MS: 687 (M+NH₄)⁺.

(f) In an analogous manner to that described in Example 1 (g), by alkylating tert-butyl (3SR,4RS,5RS)-4-[4-(2-benzyloxy-ethoxymethyl)-phenyl]-3-hydroxymethyl-5-(naphthalen-2-ylmethoxy)-piperidine-1-carboxylate with rac-2-(3-bromopropoxy)-tetrahydro-2H-pyran (J. Org. Chem. 53, (1988), 25, 5903-5908] there was obtained a mixture of tert-butyl (3RS,4RS,5SR)- and (3SR,4SR,5RS)-4-[4-(2-benzyloxy-ethoxymethyl)-phenyl]-3-(naphthalen-2-ylmethoxy)-5-[3-[(RS)-tetrahydro-pyran-2-yloxy]-propoxymethyl]-piperidine-1-carboxylate as a colorless oil; MS: 771 (M+NH₄)⁺.

(g) In an analogous manner to that described in Example 24 (m), reaction of tert-butyl (3RS,4RS,5SR)-4-(4-hydroxymethyl-phenyl)-3-(naphthalen-2-ylmethoxy)-5-trityloxymethyl-piperidine-1-carboxylate with phenyl isocyanate gave (3RS,4RS,5SR)-3-(naphthalen-2-ylmethoxy)-4-(4-phenylcarbamoyloxymethyl-phenyl)-5-trityloxymethyl-piperidine-1-carboxylate, from which by cleavage of the trityl group by means of a mixture of trifluoroacetic acid and trifluoroacetic anhydride in methylene chloride analogously to Example 86 (u) there was obtained tert-butyl (3SR,4RS,5RS)-3-hydroxymethyl-5-(naphthalen-2-ylmethoxy)-4-(4-phenylcarbamoyloxymethyl-phenyl)-piperidine-1-carboxylate. Subsequent alkylation with methyl iodide analogously to Example 1 (g) yielded tert-butyl (3SR,4RS,5RS)-3-methoxymethyl-4-{4-[(methyl-phenyl-carbamoyloxy)-methyl]-phenyl}-5-(naphthalen-2-ylmethoxy)-piperidine-1-carboxylate as a colorless oil; MS: 642 (M+NH₄)⁺.

(h) In an analogous manner to that described in Example 24 (m), reaction of tert-butyl (3RS,4RS,5SR)-4-(4-hydroxymethyl-phenyl)-3-(naphthalen-2-ylmethoxy)-5-trityloxymethyl-piperidine-1-carboxylate with benzyl isocyanate gave tert-butyl (3RS,4RS,5SR)-4-(4-benzylcarbamoyloxymethyl-phenyl)-3-(naphthalen-2-ylmethoxy)-5-trityloxymethyl-piperidine-1-carboxylate, from which by cleavage of the trityl group by means of a mixture of trifluoroacetic acid and trifluoroacetic anhydride in methylene chloride analogously to Example 86 (u) there was obtained tert-butyl (3SR,4RS,5RS)-4-(4-benzylcarbamoyloxymethyl-phenyl)-3-hydroxymethyl-5-(naphthalen-2-ylmethoxy)-piperidine-1-carboxylate. Subsequent alkylation with methyl iodide analogously to Example 1 (g) yielded tert-butyl (3SR,4RS,5RS)-4-[4-[(benzyl-methyl-carbamoyloxy)-methyl]-phenyl]-3-methoxymethyl-5-(naphthalen-2-ylmethoxy)-piperidine-1-carboxylate as a colorless oil; MS: 656 (M+NH₄)⁺.

### Example 152

The following compounds were obtained by cleavage of the BOC group by means of zinc bromide in methylene chloride:

1)—From tert-butyl (3RS,4RS)-4-(4-fluoro-phenyl)-3-(4-methylsulfanyl-benzyloxy)-piperidine-1-carboxylate, (3RS,4RS)-4-(4-fluoro-phenyl)-3-(4-methylsulfanyl-benzyloxy)-piperidine as a yellowish oil; MS: 332 (M+H)⁺;

2)—from a mixture of tert-butyl (3RS,4RS)- and (3SR,4SR)-4-(4-fluoro-phenyl)-3-[(RS)-4-methansulfinyl-benzyloxy)-piperidine-1-carboxylate, a mixture of (3RS,4RS)- and (3SR,4SR)-4-(4-fluoro-phenyl)-3-[(RS)-4-methylsulfinyl-benzyloxy]-piperidine as a yellowish oil; MS: 348 (M+H)⁺;

3)—from tert-butyl (3RS,4RS)-4-(4-fluoro-phenyl)-3-(4-methylsulfonyl-benzyloxy)-piperidine-1-carboxylate, (3RS,4RS)-4-(4-fluoro-phenyl)-3-(4-methylsulfonyl-benzyloxy)-piperidine hydrobromide as a yellowish solid; MS: 364 (M+H)⁺;

4)—from tert-butyl (3RS,4RS)-3-(1,4-dimethoxy-naphthalen-2-ylmethoxy)-4-(4-hydroxy-phenyl)-piperidine-1-carboxylate, (3RS,4RS)-4-[3-(1,4-dimethoxy-naphthalen-2-ylmethoxy)-piperidin-4-yl]-phenol as a colorless solid; MS: 394 (M+H)⁺;

5)—from tert-butyl (3RS,4RS)-4-[4-(3-Cyano-benzyloxy)-phenyl]-3-(1,4-dimethoxy-naphthalen-2-ylmethoxy)-piperidine-1-carboxylate, by means of zinc bromide, (3RS,4RS)-3-[4-[3-(1,4-dimethoxy-naphthalen-2-ylmethoxy)-piperidin-4-yl]-phenoxymethyl]-benzonitrile as a colorless solid; MS: 509 (M+H)⁺.

The BOC derivatives used as starting materials were prepared as follows:

(a) In an analogous manner to that described in Example 1 (g), by alkylating tert-butyl (3RS,4RS)-4-(4-fluoro-phenyl)-3-hydroxy-piperidine-1-carboxylate [Example 3 (b)] with 4-methylthio-benzyl chloride [J. Org. Chem. (1988), 53(3), 561-569] there was obtained tert-butyl (3RS,4RS)-4-(4-fluoro-phenyl)-3-(4-methylsulfanyl-benzyloxy)-piperidine-1-carboxylate as a colorless oil; MS: 432 (M+H)⁺.

(b) In an analogous manner to that described in Example 58 (i), by oxidizing tert-butyl (3RS,4RS)-4-(4-fluoro-phenyl)-3-(4-methylsulfanyl-benzyloxy)-piperidine-1-carboxylate by means of sodium metaperiodate there was obtained a mixture of (3RS,4RS)- and (3SR,4SR)-4-(4-fluoro-phenyl)-3-[(RS)-4-methanesulfinyl-benzyloxy)-piperidine-1-carboxylate as a yellowish oil; MS: 448 (M+H)⁺.

(c) 126 mg (0.586 mmol) of m-perbenzoic acid (80%) were added to a solution of 115 mg (0.27 mmol) of tert-butyl (3RS,4RS)-4-(4-fluoro-phenyl)-3-(4-methylsulfanyl-benzyloxy)-piperidine-1-carboxylate in 2 ml of methylene chloride. The reaction solution was stirred at room temperature for 2 hours and thereafter neutralized with potassium carbonate in methanol. Subsequently, the solution was diluted with methylene chloride. Thereafter, it was worked-up in the usual manner and the crude product obtained was purified by chromatography on silica gel using a 4:1 mixture of methylene chloride and ether as the eluent. There were obtained 1.05 mg (85% of theory) of tert-butyl (3RS,4RS)-4-(4-fluoro-phenyl)-3-(4-methylsulfonyl-benzyloxy)-piperidine-1-carboxylate as a colorless, viscous oil; MS: 464 (M+H)⁺.

(d) (α) In an analogous manner to that described in Example 1 (g), by alkylating tert-butyl (3RS,4RS)-4-(4-allyloxy-phenyl)-3-hydroxy-piperidine-1-carboxylate [Example 86 (b)] with 2-chloromethyl-1,4-dimethoxy-naphthalene [J. Org. Chem. (1983), 48(19),3265-3268) there was obtained tert-butyl (3RS,4RS)-4-(4-allyloxy-phenyl)-3-(1,4-dimethoxy-naphthalen-2-ylmethoxy)-piperidine-1-carboxylate as a pale yellow solid; MS: 534 (M+H)⁺.

(β) A solution of 315 mg (0.59 mmol) of tert-butyl (3RS,4RS)-4-(4-allyloxy-phenyl)-3-(1,4-dimethoxy-naphthalen-2-ylmethoxy)-piperidine-1-carboxylate, 44 mg (0.059 mmol) of bis-(triphenylphosphine)-palladium(II) diacetate and 132 mg (1.18 mmol) of 1,4-diazabicyclo [2.2.2]octane in 10 ml of 95% ethanol was heated to reflux under argon for 2 hours. Subsequently, the reaction mixture was cooled to 0-5° C. and treated with 0.6 ml of 1N hydrochloric acid. Thereafter, the solvent was distilled off under reduced pressure and the residue was partitioned between ethyl acetate and water. After extraction and drying of the organic phase over sodium sulfate it was evaporated under reduced pressure. The residue was purified on silica gel using a 9:1 mixture of methylene chloride and ether as the eluent. There were obtained 265 mg (91% of theory) of tert-butyl (3RS,4RS)-3-(1,4-dimethoxy-naphthalen-2-ylmethoxy)-4-(4-hydroxy-phenyl)-piperidine-1-carboxylate as a yellowish foam; MS: 494 (M+H)⁺.

(e) in an analogous manner to that described in Example 44 e), by alkylating tert-butyl (3RS,4RS)-3-(1,4-dimethoxy-naphthalen-2-ylmethoxy)-4-(4-hydroxy-phenyl)-piperidine-1-carboxylate with 3-bromomethyl-benzonitrile there was obtained tert-butyl (3RS,4RS)-4-[4-(3-cyano-benzyloxy)-phenyl]-3-(1,4-dimethoxy-naphthalen-2-ylmethoxy)-piperidine-1-carboxylate as a beige solid; MS: 609 (M+H)⁺.

### Example 153

A solution of 736 mg (1.28 mmol) of (3R,4R)-4-(4-bromophenyl)-3-(naphthalen-2-ylmethoxy)-1-[(1S,4R)-4,7,7-trimethyl-3-oxo-2-oxabicyclo[2.2.1]heptane-1-carbonyl]-piperidine in 32 ml of absolute tetrahydrofuran was cooled to −70° C. Thereto there was added dropwise a solution of 1.86 ml (6.5 mmol) of sodium dihydrido-bis-(2-methoxyethoxy)-aluminate (70% in toluene, about 3.5M) in 32 ml of tetrahydrofuran. The mixture was warmed to −40° C., stirred at −40° C. for 8 hours, thereafter cooled to −78° C. and treated dropwise with a solution of 0.5 ml of glacial acetic acid in 10 ml of tetrahydrofuran. For the working-up, the reaction mixture was partitioned between 200 mg of ethyl acetate and 200 ml of aqueous 5% sodium hydrogen carbonate solution. The organic phase was separated and the aqueous phase was back-extracted three times with 50 ml of ethyl acetate each time. The combined organic phases were dried over magnesium sulfate and finally the solvent was distilled off under reduced pressure. The crude product (800 mg) was purified by chromatography on silica gel with a 9:1 mixture of methylene chloride and methanol as the eluent. There were obtained 344 mg (68% of theory) of (3R,4R)-4-(4-bromophenyl)-3-(naphthalen-2-ylmethoxy)-piperidine as a colorless solid; MS: 396, 398 (M+H)⁺.

The camphanic acid derivative used as the starting material was prepared as follows:

A solution of 1.98 g (5.00 mmol) of (3RS,4RS)-4-(4-bromophenyl)-3-(naphthalen-2-ylmethoxy)-piperidine (Example 2.6) in 20 ml of methylene chloride was cooled to 0° C. and treated with 0.84 ml (0.61 g, 6.0 mmol) of triethylamine. Thereto there was added dropwise under argon at 0° C. a solution of 1.19 g (5.5 mmol) of (−)-(1S,4R)-camphanoyl chloride in 20 ml of methylene chloride and the mixture was subsequently stirred at 0° C. for 30 minutes and at room temperature for a further 2 hours. For the working-up, the reaction mixture was partitioned between 200 ml of methylene chloride and 200 ml of ice-water. The organic phase was separated and the aqueous phase was extracted three times with 50 ml of ethyl acetate each time. The combined organic phases were dried over magnesium sulfate and finally the solvent was distilled off under reduced pressure. The crude isomer mixture (2.89 g) was separated by chromatography on silica gel with a 2:3 mixture of ethyl acetate and hexane as the eluent. There were obtained 1.14 g (40% of theory) of (3R,4R)-4-(4-bromophenyl)-3-(naphthalen-2-ylmethoxy)-1-[(1S,4R)-4,7,7-trimethyl-3-oxo-2-oxabicyclo[2.2.1]heptane-1-carbonyl]-piperidine, MS: 575, 577 (M)⁺, and 1.01 g (35% of theory of (3S,4S)-4-(4-bromophenyl)-3-(naphthalen-2-ylmethoxy)-1-[(1S,4R)-4,7,7-trimethyl-3-oxo-2-oxabicyclo[2.2.1]heptane-1-carbonyl]-piperidine, MS: 575, 577 (M)⁺, each as a colorless solid.

### Example 154

The following compounds were obtained in an analogous manner to that described in the foregoing Example by reductive cleavage of the camphanyl group:

1)—From (3S,4S)-4-(4-bromophenyl)-3-(naphthalen-2-ylmethoxy)-1-[(1S,4R)-4,7,7-trimethyl-3-oxo-2-oxabicyclo-[2.2.1]heptane-1-carbonyl]-piperidine, (3S,4S)-4-(4-bromophenyl)-3-(naphthalen-2-ylmethoxy)-piperidine as a colorless solid; MS: 396, 398 (M+H)⁺;

2)—from tert-butyl (3R,4R)-4-(4-chlorophenyl)-3-(4-methoxy-benzyloxy)-1-[(1S,4R)-4,7,7-trimethyl-3-oxo-2-oxabicyclo[2.2. I]heptane-1-carbonyl)-piperidine, (3R,4R)-4-(4-chlorophenyl)-3-(4-methoxy-benzyloxy)-piperidine as a colorless solid; MS: 332, 334 (M+H)⁺;

3)—from tert-butyl (3S,4S)-4-(4-chlorophenyl)-3-(4-methoxy-benzyloxy)-1-[(1S,4R)-4,7,7-trimethyl-3-oxo-2-oxabicyclo[2.2.1]heptane-1-carbonyl]-piperidine, (3S,4S)-4-(4-chlorophenyl)-3-(4-methoxy-benzyloxy)-piperidine as a colorless solid; MS: 332, 334 (M+H)⁺;

4)—from tert-butyl (3R,4R)-4-naphthalen-1-yl-3-(naphthalen-2-ylmethoxy)-1-[(1S,4R)-4,7,7-trimethyl-3-oxo-2-oxabicyclo[2.2.1]heptane-1-carbonyl]-piperidine, (3R,4R)-4-naphthalen-1-yl-3-(naphthalen-2-ylmethoxy)-piperidine as a colorless solid; MS: 368 (M+H)⁺;

5)—from tert-butyl (3S,4S)-4-naphthalen-1-yl-3-(naphthalen-2-ylmethoxy)-1-[(1S,4R)-4,7,7-trimethyl-3-oxo-2-oxabicyclo[2.2.1]heptane-1-carbonyl]-piperidine, (3S,4S)-4-naphthalen-1-yl-3-(naphthalen-2-ylmethoxy)-piperidine as a colorless solid; MS: 368 (M+H)⁺;

6)—from tert-butyl (3R,4R)-3-(4-benzyloxy-naphthalen-2-ylmethoxy)-4-(4-fluoro-phenyl)-1-[(1S,4R)-4,7,7-trimethyl-3-oxo-2-oxabicyclo[2.2.1]heptane-1-carbonyl]-piperidine, (3R,4R)-3-(4-benzyloxy-naphthalen-2-ylmethoxy)-4-(4-fluoro-phenyl)-piperidine as a colorless solid; MS: 441 (M)⁺;

7)—from tert-butyl (3S,4S)-3-(4-benzyloxy-naphthalen-2-ylmethoxy)-4-(4-fluoro-phenyl)-1-[(1S,4R)-4,7,7-trimethyl-3-oxo-2-oxabicyclo[2.2.1]heptane-1-carbonyl]-piperidine, (3S,4S)-3-(4-benzyloxy-naphthalen-2-ylmethoxy)-4-(4-fluoro-phenyl) piperidine as a colorless solid; MS: 441 (M)⁺.

The following derivatives were prepared in an analogous manner to that described in the preceding Example by acylation with (−)-camphanoyl chloride:

(a)—From (3RS,4RS)-4-(4-chlorophenyl)-3-(4-methoxy-benzyloxy)-piperidine [prepared as described in den Examples 1 and 2 for (3RS,4RS)-3-(4-methoxy-benzyloxy)-4-p-tolyl-piperidine (Example 2.4)], (3R,4R)-4-(4-chlorophenyl)-3-(4-methoxy-benzyloxy)-1-[(1S,4R)-4,7,7-trimethyl-3-oxo-2-oxabicyclo[2.2.1]heptane-1-carbonyl]-piperidine, MS: 511, 513 (M)⁺, and (3S,4S)-4-(4-chlorophenyl)-3-(4-methoxy-benzyloxy)-1-[(1S,4R)-4,7,7-trimethyl-3-oxo-2-oxabicyclo[2.2.1]heptane-1-carbonyl)-piperidine, MS: 511, 513 (M)⁺, each as a colorless solid;

(b)—from tert-butyl (3RS,4RS)-4-naphthalen-1-yl-3-(naphthalen-2-ylmethoxy)-piperidine (Example 2.12), (3R,4R)-4-naphthalen-1-yl-3-(naphthalen-2-ylmethoxy)-1-[(1S,4R)-4,7,7-trimethyl-3-oxo-2-oxabicyclo[2.2.1]heptane-1-carbonyl]-piperidine, MS: 547 (M)⁺, and (3S,4S)-4-naphthalen-1-yl-3-(naphthalen-2-ylmethoxy)-1-[(1S,4R)-4,7,7-trimethyl-3-oxo-2-oxabicyclo[2.2.1]heptane-1-carbonyl]-piperidine, MS: 547 (M)⁺, each as a colorless foam;

(c)—from tert-butyl (3RS,4RS)-3-(4-benzyloxy-naphthalen-2-ylmethoxy)-4-(4-fluoro-phenyl)-piperidine (Example 14.13), (3R,4R)-3-(4-benzyloxy-naphthalen-2-ylmethoxy)-4-(4-fluoro-phenyl)-1-[(1S,4R)-4,7,7-trimethyl-3-oxo-2-oxabicyclo[2.2.1]-heptane-1-carbonyl]-piperidine, MS: 621 (M)⁺, and (3S,4S)-3-(4-benzyloxy-naphthalen-2-ylmethoxy)-4-(4-fluoro-phenyl)-1-[(1S,4R)-4,7,7-trimethyl-3-oxo-2-oxabicyclo[2.2.1]heptane-1-carbonyl]-piperidine, MS: 621 (M)⁺, each as a colorless foam.

The invention has been described with reference to various specific and preferred embodiments and techniques. However, it should be understood that many variations and modifications may be made while remaining within the spirit and scope of the invention.

1. A method of treating or preventing Alzheimer's disease in a patient in need of such treatment comprising administering a therapeutically effective amount of a compound of Formula (I) or a pharmaceutically acceptable salt thereof: 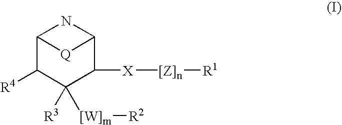
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C)C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶—, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶CO—O, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹)—, where R¹¹ is as defined above,
(M) —N(R¹¹)—CO—, where R¹¹ is as defined above,
(N) —N(R¹¹)—CO—N(R¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹)—, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

2. A method of treating Alzheimer's disease in a patient in need of such treatment comprising administering to the patient a compound disclosed in claim 1, or a pharmaceutically acceptable salt thereof.

3. A method of treating Alzheimer's disease by modulating the activity of beta amyloid converting enzyme, comprising administering to a patient in need of such treatment a compound disclosed in claim 1, or a pharmaceutically acceptable salt thereof.

4. The method according to claim 1, further comprising the administration of a P-gp inhibitor, or a pharmaceutically acceptable salt thereof.

5. A method of treating a patient who has, or in preventing a patient from getting, a disease or condition selected from the group consisting of Alzheimer's disease, for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (I), or a pharmaceutically acceptable salt thereof: 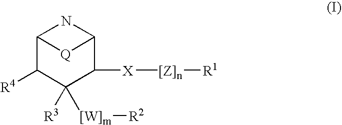
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C) C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶—, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹)—, where R¹¹ is as defined above,
(M) —N(R¹¹)—CO—, where R¹¹ is as defined above,
(N) —N(R¹¹)—CO—N(R¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹)—, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

6. The method according to claim 5 wherein the compound of formula (I) is selected from the group consisting of:
4-[2-[7-[(3R,4R)-4-[4-(3-benzyloxy-propoxy)-phenyl]-piperidin-3-yloxymethyl]-naphthalen-2-yloxy]-ethyl]-morpholine;
(R)-3-[7-[(3R,4R)-4-[4-(3-benzyloxy-propoxy)-phenyl]-piperidin-3-yloxymethyl]-naphthalen-2-yloxy]-propane-1,2-diol;
(S)-3-[7-[(3R,4R)-4-[4-(3-benzyloxy-propoxy)-phenyl]-piperidin-3-yloxymethyl]-naphthalen-2-yloxy]-propane-1,2-diol;
(R)-3-[2-[7-[(3R,4R)-4-[4-(3-benzyloxy-propoxy)-phenyl]-piperidin-3-yloxymethyl]-naphthalen-2-yloxy]-ethoxy]-propane-1,2-diol;
(S)-3-[2-[7-[(3R,4R)-4-[4-(3-benzyloxy-propoxy)-phenyl]-piperidin-3-yloxymethyl]-naphthalen-2-yloxy]-ethoxy]-propane-1,2-diol;
1-[2-[7-[(3R,4R)-4-[4-(3-benzyloxy-propoxy)-phenyl]-piperidin-3-yloxymethyl]-naphthalen-2-yloxy]-ethyl]-4-methyl-piperazine;
1-[(3R,4S-4-[4-(3-benzyloxy-propoxy)-phenyl]-piperidin-3-yl-2-naphthalen-2-yl-ethanone;
(3R,4S,5 S)-4-[4-(3-benzyloxy-propoxy)-phenyl]-3-(1,4-dimethoxy-naphthalen-2-ylmethoxy)-piperidin-5-ol;
3R,4R)-4-[4-(3-benzyloxy-propoxy)-phenyl]-3-[7-[(R)-2,3-dihydroxy-propoxymethyl]-naphthalen-2-ylmethoxy]-piperidine;
(3R,4R)-4-[4-(3-benzyloxy-propoxy)-phenyl]-3-[7-[(S)-2,3-dihydroxy-propoxymethyl]-naphthalen-2-ylmethoxy]-piperidine;
(3R,4R)-4-[4-(3-benzyloxy-propoxy)-phenyl]-3-[6-[(R)-2,3-dihydroxy-propoxymethyl]-naphthalen-2-ylmethoxy]-piperidine;
(3R,4R)-4-[4-(3-benzyloxy-propoxy)-phenyl]-3-[6-[(S)-2,3-dihydroxy-propoxymethyl]-naphthalen-2-ylmethoxy]-piperidine;
4-[(3R,4S,5 S)-4-[4-(3-benzyloxy-propoxy)-phenyl]-3-(naphthalen-2-ylmethoxy)-piperidin-5-yloxy]-butan-1-ol;
3-[(3R,4S,5 S)-4-[4-(3-benzyloxy-propoxy)-phenyl]-3-(naphthalen-2-ylmethoxy)-piperidin-5-yloxy]-propan-1-ol;
1-{2-[(3R,4R,5 S)-4-[4-(3-benzyloxy-propoxy)-phenyl]-3-(naphthalen-2-ylmethoxy)-piperidin-5-yloxy]-ethyl}-4-methyl-piperazine;
(3R,4R,5S)-[4-[4(3-benzyloxy-propoxy)-phenyl]-3-(naphthalen-2-ylmethoxy)-piperidin-5-ylmethoxy]-ethyl]-morpholine;
(3R,4S,5 S)-4-[4-(3-benzyloxy-propoxy)-phenyl]-3-(4-methoxy-benzyloxy)-piperidin-5-ol;
(3R,4s,5S)-4-[4-(3-benzyloxy-propoxy)-phenyl]-3,5-bis-(4-methoxy-benzyloxy)-piperidine;
(3S,4R,5R)-4-[2-[4-[4-(3-benzyloxy-propoxy)-phenyl]-5-(naphthalen-2-ylmethoxy)-piperidin-3-ylmethoxy]-ethyl]-morpholine;
(3S,4R,5R)-4-[4-(3-benzyloxy-propoxy)-phenyl]-3-methoxymethyl-5-(naphthalen-2-ylmethoxy)-piperidine;
(3S,4R,5R)-4-[4-(3-benzyloxy-propoxy)-phenyl]-5-(naphthalen-2-ylmethoxy)-piperidin-3-ylmethyl[3-(4-methyl-piperazin-1-yl)-propyl]-carbamate;
(3S,4R,5R)-4-[4-[4-(3-benzyloxy-propoxy)-phenyl]-5-(naphthalen-2-ylmethoxy)-piperidin-3-ylmethylsulphanyl]-pyridine;
2-(4-cyclohexyl-butoxy)-5-[(3R,4R)-3-(1,4-dimethoxy-naphthalen-2-ylmethoxy)-piperidin-4-yl]-pyrimidine;
(3′R,4′R)-6-(3-cyclohexyl-propoxy)-3′-(1,4-dimethoxy-naphthalen-2-ylmethoxy)-1′,2′,3′,4′,5′,6′-hexahydro-[3,4′]bipyridine;
(3S,4R,5R)-[4-[4-(3-benzyloxy-propoxy)-phenyl]-5-(naphthalen-2-ylmethoxy)-piperidin-3-yl]-methanol;
(3S,4R,5R)-N-[4-[4-(3-benzyloxy-propoxy)-phenyl]-5-(naphthalen-2-ylmethoxy)-piperidin-3-ylmethyl]-N,N′,N′-trimethyl-ethane-1,2-diamine;
(3S,4R,5R)-[4-[4-(3-benzyloxy-propoxy)-phenyl]-5-(naphthalen-2-ylmethoxy)-piperidin-3-ylmethyl]-diethyl-amine;
1-[(3R,4S,5S)-4-[4-(3-benzyloxy-propoxy)-phenyl]-5-(2-morpholin-4-yl-ethoxymethyl)-piperidin-3-yl]-2-naphthalen-2-yl-ethanone;
(3R,4R)-3-(1,4-dimethoxy-naphthalen-2-ylmethoxy)-4-[4-[3-(2-methoxy-phenoxy)-propoxy]-phenyl]-piperidine;
(3R,4s,5S)-4-[4-(3-benzyloxy-propoxy)-phenyl]-3,5-bis-(3,4,5-trimethoxy-benzyloxy)-piperidine;
(3R,4R,5S)-4-[4-(3-benzyloxy-propoxy)-phenyl]-3-(naphthalen-2-ylmethoxy)-5-[1,2,4]triazol-1-ylmethyl-piperidine;
(3R,4R)-4-[4-[3-(2-methoxy-benzyloxy)-propoxy]-phenyl]-3-(quinolin-7-ylmethoxy)-piperidine;
2-(7-{(3R,4R)-4-[4-(3-benzyloxy-propoxy)-phenyl]-piperidin-3-yloxymethyl}-naphthalen-2-ylmethoxy)-ethanol;
7-{(3R,4R)-4-[4-(3-benzyloxy-propoxy)-phenyl]-piperidin-3-yloxymethyl}-naphthalen-2-ylmethyl)-dimethyl-amine;
(3R,4R)-3-(4-benzyloxy-naphthalen-2-ylmethoxy)-4-(4-fluoro-phenyl)-piperidine;
(3′R,4′R)-3′-(1,4-dimethoxy-naphthalen-2-ylmethoxy)-6-[3-(2-methoxybenzyloxy)-propoxy]-1′,2′,3′,4′,5′,6′-hexahydro-[3,4′]bipyridine;
(3R,4R)-3-(1,4-dimethoxy-naphthalen-2-ylmethoxy)-4-[4-[3-(2-methoxy-benzyloxy)-propoxy]-phenyl]-piperidine;
(3S,4R,5R)-1-[4-[4-(3-benzyloxy-propoxy)-phenyl]-5-(naphthalen-2-ylmethoxy)-piperidin-3-ylmethyl]-imidazolidin-2-one;
(3R,4R)-4-[4-[3-(2-methoxy-benzyloxy)-propoxy]-phenyl]-3-(2-oxo-1,2-dihydro-quinolin-7-ylmethoxy)-piperidine;
(3R,4R)-3-(isoquinolin-7-ylmethoxy)-4-[4-[3-(2-methoxy-benzyloxy)-propoxy]-phenyl]-piperidine;
(3R,4R)-4-[4-[3-(2-methoxy-benzyloxy)-propoxy]-phenyl]-3-(1,2,3,4-tetrahydro-quinolin-7-ylmethoxy)-piperidine;
1-[2-[7-[(3R,4R)-4-[4-[3-(2-methoxy-benzyloxy)-propoxy]-piperidin-3-yloxymethyl]-naphthalen-2-yloxy]-ethyl]-4-methyl-piperazine;
1-[2-[7-[(3R,4S,5 S)-5-hydroxy-4-[4-[3-(2-methoxy-benzyloxy)-propoxy]-piperdin-3-yloxymethyl]-naphthalen-2-yloxy]-ethyl]-4-methyl-piperazine;
(3R,4S,5S)-3-(1,4-dimethoxy-naphthalen-2-ylmethoxy)-4-[4-[3-(2-methoxy-benzyloxy)-propoxy]-phenyl]-piperidin-5-ol;
(3R,4R,5S)-3-(1,4-dimethoxy-naphthalen-2-ylmethoxy)-4-{4-[3-(2-methoxy-benzyloxy)-propoxy]-phenyl}-5-(1H-tetrazol-5-ylmethyl)-piperidine;
(3′S,4′S)-3′-(1,4-dimethoxy-naphthalen-2-ylmethyoxy)-4-[S-(2-methoxy-benzyloxy)-propoxy]-1′,2′,3′,4′,5′,6′-hexahydro-[1,4′]bipyridin-2-one;
(3RS,4RS)-2-[4(3-Naphthalen-2-ylmethoxy-piperidin-4-yl)-phenoxy]-ethylthiophene-2-carboxylate hydrochloride;
(3RS,4RS)-2-[4(3-Naphthalen-2-ylmethoxy-piperidin-4-yl)-phenoxy]-ethyl 2-chloro-benzoate hydrochloride;
(3RS,4RS)-2-[4[3[4(2-methoxy-benzyloxy)-naphthalen-2-ylmethoxy]-piperidin-4-yl]-phenoxy]-ethyl benzoate hydrochloride;
(3RS,4RS)-4-[4(3-Benzyloxy-propoxy)-phenyl]3-(2,3-dihydro-benzo[1,4]dioxin-6-ylmethoxy)-piperidine;
(3RS,4RS)-3-(Naphthalen-2-ylmethoxy)-4-[4(3-phenyl-[1,2,4]oxadiazol-5-ylmethoxy)-phenyl]-piperidine trifluoroacetate;
(3RS,4RS)-3-(Naphthalen-2-ylmethoxy)-4-[4(3-phenyl-isoxazol-5-ylmethoxy)-phenyl]-piperidine trifluoroacetate;
(3RS,4RS)-3-(Naphthalen-2-ylmethoxy)-4-[4(3-phenylsulphanyl-propyl)-phenyl]-piperidine;
(3RS,4RS)-3-[4[4[2(Benzothiazol-2-ylsulphanyl)-ethyl]-phenyl]-piperidin-3-yloxymethyl]-naphthalen-1-ol;
(3RS,4RS,5 SR)-3-(4-Benzyloxy-naphthalen-2-ylmethoxy)-4-(4-fluoro-phenyl)-5-propyl-piperidine;
(3SR,4RS,5RS)-4-(4-Benzyloxymethyl-phenyl)-3-methoxymethyl-5-(naphthalen-2-ylmethoxy)-piperidine;
(SR)- or (RS)-1-[(3RS,4SR)4-(4-fluoro-phenyl)-piperidin-3-yl]-2-naphthalen-2-yl-ethyl benzoate hydrochloride;
(1RS,2RS,3RS,5 SR)-2-(4-Benzyloxy-naphthalen-2-ylmethoxy)-3-(4-fluoro-phenyl)-8-aza-bicyclo[3.2.1]octane;
(3RS,4RS)-4-[4-(3-Benzyloxy-propoxy)-phenyl]-3-(naphthalen-2-ylmethoxy)-piperdine;
4-[2-[7-[(3RS,4RS)-4-[4(3-Benzyloxy-propoxy)-phenyl]piperidin-3-yloxymethyl]-naphthalen-2-yloxy]-ethyl]-morpholine hydrochloride (1:2);
3-[7-[(3RS,4RS)-4-[4(3-benzyloxy-propoxy)-phenyl]-piperidin-3-yloxymethyl]-naphthalen-2-yloxy]-propane-1,2-diol;
(RS)- and (SR)-3-[2-[7-[(3RS,4RS)-4-[4(3-benzyloxy-propoxy)-phenyl]-piperidin-3-yloxymethyl]-naphthalen-2-yloxy]-ethoxy]-propane-1,2-diol hydrochloride (1:1);
1-[2-[7-[(3RS,4RS)-4-[4-(3-Benzyloxy-propoxy)-phenyl]-piperidin-3-yloxymethyl]-naphthalen-2-yloxy]-ethyl-4-methyl-piperazine hydrochloride (1:3);
1-[(3RS,4SR)-4-[4-(3-Benzyloxy-propoxy)-phenyl]-piperidin-3-yl]-2-naphthalen-2-yl-ethanone hydrochloride (1:1);
(3RS,4SR,5SR)-4-[4(3-Benzyloxy-propoxy)-phenyl]-3-(1,4-dimethoxy-naphthalen-2-ylmethoxy)-piperidin-5-ol;
4-[4-(3-benzyloxy-propoxy)-phenyl]-3-[7-[(RS)2,3-dihydroxy-propoxymethyl]naphthalen-2-ylmethoxy]-piperidine;
4-[4-(3-benzyloxy-propoxy)-phenyl]-3-[6-[(RS)-2,3-dihydroxy-propoxymethyl]-naphthalen-2-ylmethoxy]-piperidine;
4-[(3RS,4SR,5SR)-4-[4(3-Benzyloxy-propoxy)-phenyl]-3-(naphthalen-2-ylmethoxy)-piperidin-5-yloxy]-butan-1-ol;
3-[(3RS,4SR,5SR)-4-[4(3-Benzyloxy-propoxy)-phenyl]-3-(naphthalen-2-ylmethoxy)-piperidin-5-yloxy]-propan-1-ol;
1-{2-[(3RS,4RS,5SR)-4-[4-(3-Benzyloxy-propoxy)-phenyl]-3-(naphthalen-2-ylmethoxy)-piperidin-5-yloxy]-ethyl}-4-methyl-piperazine;
4-{2-[(3RS,4RS,5SR)-4-[4(3-Benzyloxy-propoxy)-phenyl]-3-(naphthalen-2-ylmethoxy)-piperidin-5-yloxy]-ethyl}-morpholine;
(3RS,4SR,5SR)-4-[4(3-Benzyloxy-propoxy)-pheny]-l3-(4-methoxy-benzyloxy)-piperidin-5-ol;
(3R,4s,5S)-4-[4(3-Benzyloxy-propoxy)-phenyl]-3,5-bis-(4-methoxy-benzyloxy)-piperidine;
(3SR,4RS,5RS)-4-[2-[4-[4-(3-Benzyloxy-propoxy)-phenyl]-5-(naphthalen-2-ylmethoxy)-piperidin-3-ylmethoxy]-ethyl]-morpholine;
(3SR,4RS,5RS)-4-[4-(3-Benzyloxy-propoxy)-phenyl]-3-methoxymethyl-5-(naphthalen-2-ylmethoxy)-piperidine;
(3SR,4RS,5RS)-4-[4-(3-Benzyloxy-propoxy)-phenyl]-5-(naphthalen-2-ylmethoxy)-piperidin-3-ylmethyl[3 (4-methyl-piperazin-1-yl)-propyl]-carbamate;
(3SR,4RS,5RS)-4-[4-[4-(3-Benzyloxy-propoxy)-phenyl]-5-(naphthalen-2-ylmethoxy)-piperidin-3-ylmethylsulphanyl]-pyridine;
2-(4-Cyclohexyl-butoxy)-5-[(3RS,4RS)-3-(1,4-dimethoxy-naphthalen-2-ylmethoxy)-piperidin-4-yl]-pyrimidine;
(3′RS,4′RS)-6-(3-Cyclohexyl-propoxy)-3′-(1,4-dimethoxy-naphthalen-2-ylmethoxy)-1′,2′,3′,4′,5′,6′-hexahydro-[3,4′]-bipyridine;
(3SR,4RS,5RS)-[4-[4-(3-Benzyloxy-propoxy)-phenyl]-5-(naphthalen-2-ylmethoxy)-piperidin-3-yl]-methanol hydrochloride;
(3SR,4RS,5RS)-N-[4-[4-(3-Benzyloxy-propoxy)-phenyl]-5-(naphthalen-2-ylmethoxy)-piperidin-3-ylmethyl]-N,N′,N′-trimethyl-ethane-1,2-diamine;
(3SR,4RS,5RS)-[4-[4-(3-Benzyloxy-propoxy)-phenyl]-5-(naphthalen-2-ylmethoxy)-piperidin-3-yl]-methyldiethyl-amine;
1-[(3RS,4SR,5SR)-4-[4-(3-Benzyloxy-propoxy)-phenyl]-5-(2-morpholin-4-yl-ethoxymethyl)-piperidin-3-yl]-2-naphthalen-2-yl-ethanone;
(3RS,4RS)-3-(1,4-Dimethoxy-naphthalen-2-ylmethoxy)-4-[4-[3-(2-methoxy-phenoxy)-propoxy]-phenyl]-piperidine;
(3R,4s,5S)-4-[4-(3-Benzyloxy-propoxy)-phenyl]-3,5-bis-(3,4,5-trimethoxy-benzyloxy)-piperidine;
(3RS,4RS,5SR)-4-[4-(3-Benzyloxy-propoxy)-phenyl]-3-(naphthalen-2-ylmethoxy)-5-[1,2,4]triazol-1-ylmethyl-piperidine hydrochloride;
(3RS,4RS)-4-[4-[3-(2-Methoxy-benzyloxy)-propoxy]-phenyl]-3-(quinolin-7-ylmethoxy)-piperidine;
2-(7-{(3RS,4RS)-4-[4(3-Benzyloxy-propoxy)-phenyl]-piperidin-3-yloxymethyl}-naphthalen-2-ylmethoxy)-ethanol;
7-{(3RS,4RS)-4-[4-(3-Benzyloxy-propoxy)-phenyl]-piperidin-3-yloxymthyl}-naphthalen-2-ylmethyl)-dimethyl-amine;
(3R,4R)-3-(4-Benzyloxy-naphthalen-2-ylmethoxy)-4-(4-fluoro-phenyl)-piperidine;
(3S,4S)-3-(4-Benzyloxy-naphthalen-2-ylmethoxy)-4-(4-fluoro-phenyl)-piperidine;
(3′RS,4′RS)-3′-(1,4-Dimethoxy-naphthalen-2-ylmethoxy)-6-[3-(2-methoxybenzyloxy)-propoxy]-1′,2′,3′,4′,5′,6′-hexahydro-[3,4′]bipyridine;
(3RS,4RS)-3-(1,4-Dimethoxy-naphthalen-2-ylmethoxy)-4-[4-[3-(2-methoxy-benzyloxy)-propoxy]-phenyl]-piperidine;
(3SR,4RS,5RS)-1-[4-[4-(3-Benzyloxy-propoxy)-phenyl]-5-(naphthalen-2-ylmethoxy)-piperidin-3-ylmethyl]-imidazolidin-2-one;
(3RS,4RS)-4-[4-[3-(2-Methoxy-benzyloxy)-propoxy]-phenyl]-3-(2-oxo-1,2-dihydro-quinolin-7-ylmethoxy)-piperidine;
(3RS,4RS)-3-(Isoquinolin-7-ylmethoxy)-4-[4-[3-(2-methoxy-benzyloxy)-propoxy]-phenyl]-piperidine;
(3RS,4RS)-4-[4-[3-(2-Methoxy-benzyloxy)-propoxy]-phenyl]-3-(1,2,3,4-tetrahydro-quinolin-7-ylmethoxy)-piperidine; and
(3RS,4SR,5 SR)-3-(1,4-Dimethoxy-naphthalen-2-ylmethoxy)-4-[4-[3-(2-methoxy-benzyloxy)-propoxy]-phenyl]-piperidin-5-ol.

7. A method of treating or preventing Alzheimer's disease in a patient in need of such treatment comprising administering a therapeutically effective amount of a composition comprising one or more pharmaceutically acceptable carriers and a compound of Formula (I) or a pharmaceutically acceptable salt thereof: 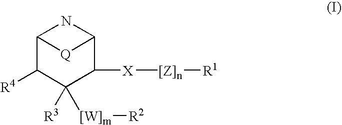
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C) C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶—, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹)—, where R¹¹ is as defined above,
(M) —N(R¹¹)—CO—, where R¹¹ is as defined above,
(N) —N(R¹¹)—CO—N(¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹)—, where R¹¹ is as defined above,
(D) —S(O)₀₋₂,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) —Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

8. (canceled)

9. A method for inhibiting beta-secretase activity, comprising contacting an effective amount for inhibition of a compound of formula (I), or a pharmaceutically acceptable salt thereof: 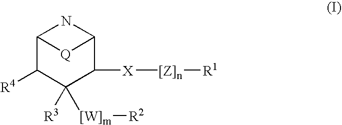
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T₁-L²-T²-L³-T³-L⁴-T⁴-L⁵-U,
where L¹, L², L³, L⁴ and L⁵ are independently chosen from:
(A) a bond,
(B) C₁₋₈-alkylene,
(C) C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹) —, where R¹¹ is as defined above,
(M) —N(R¹¹)—CO—, where R¹¹ is as defined above,
(N) —N(R¹¹)—CO—N(R¹¹) —, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹) —, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

10. A method for inhibiting cleavage of an amyloid precursor protein (APP) isotype at a site in the APP isotype that is susceptible to cleavage, comprising contacting said APP isotype with an effective cleavage inhibitory amount of a compound of formula (I), or a pharmaceutically acceptable salt thereof: 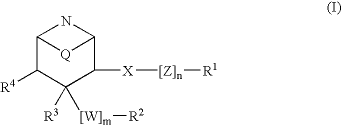
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C)C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶, where R⁶ is as defined above,
(J) —NR⁶SO₂, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹) —, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹) —, where R¹¹ is as defined above,
(M) —N(R¹¹)—CO—, where R¹¹ is as defined above,
(N) —N(R¹¹)—CO—N(R¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹) —, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

11. A method for inhibiting production of amyloid beta peptide (A beta) in a cell, comprising administering to said cell an effective inhibitory amount of a compound of formula (I), or a pharmaceutically acceptable salt thereof: 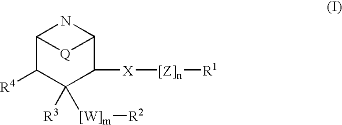
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C) C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶—, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹)—, where R¹¹ is as defined above,
(M) —N(R¹¹)—CO—, where R¹¹ is as defined above,
(N) —N(R¹¹)—CO—N(R¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹)—, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

12. The method of claim 11, wherein the cell is an animal cell.

13. The method of claim 12, wherein the animal cell is a mammalian cell.

14. The method of claim 13, wherein the mammalian cell is human.

15. A composition comprising beta-secretase complexed with a compound of formula (I), or a pharmaceutically acceptable salt thereof: 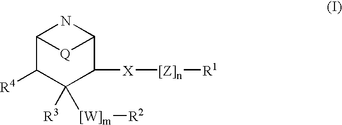
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C) C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶—, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹)—, where R¹¹ is as defined above,
(M) —N(R¹¹)—CO—, where R¹¹ is as defined above,
(N) —N(R¹¹)—CO—N(R¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹)—, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

16. A method for producing a beta-secretase complex comprising the composition of claim 15.

17. A method for inhibiting the production of beta-amyloid plaque in an animal, comprising administering to said animal an effective inhibiting amount of a compound of formula (I): 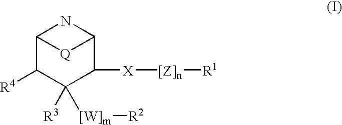
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C) C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶—, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a hetero atom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹)—, where R¹¹ is as defined above,
(M) —N(R¹¹)—CO—, where R¹¹ is as defined above,
(N) —N(R¹¹)—CO—N(R¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹)—, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

18. The method of claim 17, wherein said animal is a human.

19. A method for treating or preventing a disease characterized by beta-amyloid deposits on or in the brain, comprising administering to a patient in need of such treatment or prevention an effective therapeutic amount of a compound of formula (I): 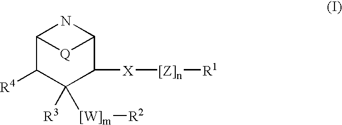
where R¹ is
(I) aryl, or
(II) heterocycle;
where R² is
(I) phenyl,
(II) naphthyl,
(III) acenaphthyl,
(IV) cyclohexyl,
(V) pyridyl,
(VI) pyrimidinyl,
(VII) pyrazinyl,
(VIII) oxo-pyridinyl,
(IX) diazinyl,
(X) triazolyl,
(XI) thienyl,
(XII) oxazolyl,
(XIII) oxadiazolyl,
(XIV) thiazolyl,
(XV) pyrrolyl, or
(XVI) furyl,
optionally substituted by one, two, or three of halogen, hydroxy, cyano, trifluoromethyl, lower-alkyl, halo-lower-alkyl, hydroxy-lower-alkyl, lower-alkoxy-lower-alkyl, cyano-lower-alkyl, carboxy-lower-alkyl, lower-alkanoyloxy-lower-alkyl, lower-alkoxycarbonyloxy-lower-alkyl, lower-alkoxycarbonyl, lower alkoxy groups, lower-alkylenedioxy, or L¹-T¹-L²-T²-L³-T³-L⁴-T⁴-L⁵-U,
where L¹, L², L³, L⁴ and L⁵ are independently chosen from:
(A) a bond,
(B) C₁₋₈-alkylene,
(C) C₂₋₈-alkenylene,
(D) C₂₋₈-alkynylene, or
(E) are absent,
where T¹, T², T³, and T⁴ are independently chosen from:
(A) a bond,
(B) —CH(OH)—,
(C) —CH(OR⁶)—, where R⁶ is hydrogen, lower-alkyl, lower-alkenyl, aryl-lower-alkyl, acyl, or together with the heteroatom to which they are attached are a 5- or 6-membered heterocyclic ring which can contain an additional N, O, or S atom or a SO or SO₂ group and the additional N atom if present can be optionally substituted by lower-alkyl,
(D) —CH(NR⁵R⁶)—, where R⁵ is as defined for R⁶, and where R⁶ is as defined above,
(E) —CO—,
(F) —CR⁷R⁸—, where R⁷ and R⁸ together with the C atom to which they are attached form a three, four, five, six, or seven membered ring which can contain one or two O or S atoms or SO or SO₂ groups,
(G) —O— or —NR⁶—, where R⁶ is as defined above,
(H) —S(O)₀₋₂—,
(I) —SO₂NR⁶—, where R⁶ is as defined above,
(J) —NR⁶SO₂—, where R⁶ is as defined above,
(K) —CONR⁶—, where R⁶ is as defined above,
(L) —NR⁶CO—, where R⁶ is as defined above,
(M) —O—CO—,
(N) —CO—O—,
(O) —O—CO—O—,
(P) —O—CO—NR⁶—, where R⁶ is as defined above,
(Q) —NR⁶—CO—NR⁶—, where R⁶ are the same or different and are as defined above,
(R) —NR⁶—CO—O—, where R⁶ is as defined above, or
(S) are absent
where the bonds emanating from (B), (D), (E) and (G)-(R) join to a C atom of the adjacent group and this C atom is saturated when the bond emanates from a heteroatom, and not more than two of (B)-(F), three of (G)-(H) or one of (I)-(R) are present,
where U is:
(A) hydrogen,
(B) lower-alkyl,
(C) cycloalkyl,
(D) cyano,
(E) optionally substituted cycloalkyl,
(F) optionally substituted aryl, or
(G) optionally substituted heterocyclyl;
where R³ is:
(I) hydrogen,
(II) hydroxy,
(III) lower-alkoxy, or
(IV) lower-alkenyloxy;
where R⁴ is:
(I) hydrogen,
(II) lower-alkyl,
(III) lower-alkenyl,
(IV) lower-alkoxy,
(V) hydroxy-lower-alkyl,
(VI) lower-alkoxy-lower-alkyl,
(VII) benzyl,
(VIII) oxo, or
(IX) where R³ and R⁴ together are a bond, or R^{4a}-Z¹-X¹—
where R^{4a} is
(A) H—,
(B) lower-alkyl-,
(C) lower-alkenyl-,
(D) hydroxy-lower-alkyl-,
(E) polyhydroxy-lower-alkyl-,
(F) lower-alkyl-O-lower-alkyl-,
(G) aryl-,
(H) heterocyclyl-,
(I) arylalkyl-,
(J) heterocyclyloxylalkyl-,
(K) aryloxyalkyl-,
(L) heterocyclyloxylalkyl-,
(M) (R⁵R⁶)—N—(CH₂)₁₋₃—, where R⁵ and R⁶ are as defined above,
(N) (R⁵R⁶)—N—, where R⁵ and R⁶ are as defined above,
(O) lower-alkyl-S(O)₀₋₂—,
(P) aryl-S(O)₀₋₂—,
(Q) heterocyclyl-S(O)₀₋₂—,
(R) HO—SO₃— or a salt thereof,
(S) H₂N—C(NH)—NH—, or
(T) NC—,
where the bonds emanating from (N)-(T) join to a C atom of the adjacent group and this C atom is saturated when the bond emanates from a heteroatom,
where Z¹ is:
(A) a bond,
(B) lower-alkylene-,
(C) lower-alkenylene-,
(D) —O—,
(E) —N(R¹¹)—, where R¹¹ is hydrogen or lower-alkyl,
(F) —S(O)₀₋₂—,
(G) —CO—,
(H) —O—CO—,
(I) —O—CO—O—,
(J) —O—CO—N(R¹¹)O, where R¹¹ is as defined above,
(K) —N(R¹¹)—CO—O—, where R¹¹ is as defined above,
(L) —CO—N(R¹¹)—, where R¹¹ is as defined above,
(M) —N(R¹¹)—CO—, where R¹¹ is as defined above,
(N) —N(R¹¹)—CO—N(R¹¹)—, where R¹¹ are the same or different and are as defined above,
(O) —CH(OR⁹)—, where R⁹ is hydrogen, lower-alkyl, acyl or arylalkyl, or
(P) is absent
where the bonds emanating from (D) and (H)-(O) join to a C atom of the adjacent group and this C atom is saturated when the bond emanates from a heteroatom;
where X¹ is:
(A) a bond,
(B) —O—,
(C) —N—(R¹¹)—, where R¹¹ is as defined above,
(D) —S(O)₀₋₂—,
(E) —(CH₂)₁₋₃—, or
(F) is absent;
where Q is:
(I) ethylene, or
(II) is absent;
where X is:
(I) a bond,
(II) —O—,
(III) —S—,
(IV) —CH—R¹¹—, where R¹¹ is as defined above,
(V) —CHOR⁹—, where R⁹ is as defined above,
(VI) —O—CO,
(VII) —CO—, or
(VIII) C═NOR¹⁰—, where R¹⁰ is carboxyalkyl, alkoxycarbonylalkyl, alkyl or hydrogen, with the bond emanating from an oxygen or sulfur atom joining to a saturated C atom of group Z or to R¹;
where W is:
(I) —O—, or
(II) —S—;
where Z is:
(I) lower-alkylene,
(II) lower-alkenylene,
(III) hydroxy-lower-alkylidene,
(IV) —O—,
(V) —S—,
(VI) —O-Alk-, where Alk is a lower alkylene
(VII) —S-Alk-, where Alk is as defined above,
(VIII) -Alk-O—, where Alk is as defined above, or
(IX) -Alk-S, where Alk is as defined above;
where n is:
(I) one, or
(II) zero or one when X is —O—CO; and
where m is 0 or 1;
with the provisos that
(I) X is —CH—R¹¹— and either R² contains a substituent L¹-T¹-L²-T²-L³-T³-L⁴-T⁴-L⁵-U or R⁴ is a substituent defined above other than hydrogen when Z is —O— or —S—,
(II) X is —CH—R¹¹— when Z is —O-Alk- or —S-Alk-, and
(III) Z is lower-alkenylene, -Alk-O— or -Alk-S— when X is a bond.

20. (canceled)

21. (canceled)
